(19) United States
(12) Patent Application Publication
(10) Pub. No.: US 2025/0346913 A1
(43) Pub. Date: Nov. 13, 2025

(54) NOVEL METHODS FOR IDENTIFICATION AND USE OF UPSTREAM OPEN READING FRAMES

(71) Applicant: genXtraits, Inc., Alameda, CA (US)

(72) Inventors: Roger Paul Hellens, Ngongotaha (NZ); Oliver J. Ratcliffe, Alameda, CA (US); Jeffrey M. Libby, Cupertino, CA (US)

(21) Appl. No.: 18/869,644

(22) PCT Filed: May 26, 2023
(86) PCT No.: PCT/US2023/067586

§ 371 Date: Nov. 26, 2024

Related U.S. Application Data
(60) Provisional application No. 63/346,629, filed on May 27, 2022.

(51) Int. Cl. C12N 15/82; C12N 5/04; C12N 5/10; G16B 30/00

(52) U.S. Cl. CPC C12N 15/8216; C12N 5/04; C12N 5/10; C12N 15/8269; G16B 30/00; C12N 2510/00

(57) ABSTRACT

The present description relates to methods and compositions for identifying and characterizing upstream open reading frames (uORFs) in eukaryotes, including plants, and means for using and/or modifying the uORFs to produce desirable traits. In so doing, means for producing commercially valuable plants and crops as well as the methods for making them and using them are identified. The uORFs identified and characterized with the present methods may be modified for the purpose of producing plants with modified traits. These traits may provide significant value in that they allow the plant to thrive in hostile environments. The traits may also comprise desirable morphological alterations.

### FIELD OF THE INVENTION

This description relates to the identification and utility of upstream open reading frames in a genome of a eukaryotic organism.

### BACKGROUND OF THE INVENTION

An upstream open reading frame (uORF) is a member of a class of small, conserved ORFs located upstream of protein-coding major ORFs (mORFs) in the 5′-untranslated regions (5′UTR) of mRNAs. uORFs act as cis acting elements that modify the activity of a downstream sequence that encodes a polypeptide. As such, they offer a novel opportunity to activate the expression of the downstream open reading frames encoding polypeptides of interest, through gene editing approaches that introduce mutations into the uORF sequences. Upregulation of the level of a target polypeptide can thereby be achieved by modulating the expression, for example, by the knocking-out or mutation, of a negatively acting uORF, that resides upstream of the sequence encoding the polypeptide. Not all eukaryotic genes contain uORFs and hitherto the barrier to the aforementioned approach has been in identifying the uORF sequences; existing algorithms often fail to accurately identify these elements due to their short length and also given that they are often initiated via non-AUG start codons (Hellens et al., 2016. Trend Plant Sci. 21:317-328). Herein, novel algorithms are presented that identify the presence of uORFs through analysis of so-called ribosome profiling data. Genetic modifications are then targeted to the uORF sequences to produce new desirable phenotypes that have a variety of applications depending upon the particular cell or organism. Such applications include new crop traits (e.g., increased yield, vigor, stress tolerance, delayed or accelerated flowering, altered morphology, or improved nutritional content), control of weeds and other pests through activation of gene networks that switch on cell death, activation of cell-death or tumor suppressor genes in cancerous cells, and/or production of desirable metabolites or peptides in fermentation systems.

uORFs are regulatory elements that are prevalent in eukaryotic mRNAs. uORFs are located upstream of protein-coding major ORFs (also known as long ORFs, main ORFs or mORFs) in the 5′-untranslated regions (5′UTR) of mRNAs. In some instances, uORFs are believed to modulate the translation initiation rate of downstream coding sequences (CDSs) by sequestering ribosomes. In other cases, uORFs encode evolutionarily conserved short peptides (sometimes referred to as “uPEPs”) that may function as cis-acting repressor peptides of the downstream mORF or its protein product. In many cases the actual presence of a uORF is strongly conserved across species. Thus, once a uORF has been identified in a target locus from a given species, the homologous locus from another species will typically also contain a uORF and be subject to uORF repression. Herein, the set of uORF containing loci from the model plant, Arabidopsis thaliana, is identified by application of our novel algorithm. These data now provide a roadmap for identifying the uORF containing loci from target crops based on homology searches for the polypeptides encoded by the mORFs at these loci. Thus, when a given desirable trait has been identified through overexpression of a gene in Arabidopsis, when that locus contains a uORF, the equivalent homologous gene in a target crop can be activated to obtain that desired trait by mutation of the uORF in the crop gene, which will typically reside at a similar position upstream of the mORF in the homologous locus of the target crop. A specific example concerns editing the uORF of LsGGP2, which encodes a key enzyme in vitamin C biosynthesis in lettuce, which was targeted based on the homologous gene having been demonstrated as being subject to uORF control in Arabidopsis by Laing et al (Liang et al., Plant Cell. 2015 March; 27(3): 772-786). Editing the uORF of the lettuce homolog not only increased oxidation stress tolerance, but also increased ascorbate content by ˜150% (Zhang et al. Nature Biotechnology volume 36, pages 894-898 (2018).

Genome-wide studies have revealed the widespread regulatory functions of uORFs in different species in different biological contexts (Zhang et al. 2019. Trends Biochem. Sci. 44:782-794. doi: 10.1016/j.tibs.2019.03.002). A given uORF may act as a translational control element for regulating expression of its associated downstream major open reading frame (mORF). The translational regulation of mORFs by highly conserved uORFs in response to cellular metabolite levels has been documented in plant studies (Hayden C. A. and Jorgensen R. A. 2007. BMC Biol. 5:32; Tran M. K., et al. 2008. BMC Genomics 9:361).

Various methods to identify uORFs in eukaryotes have been described. For example, to identify conserved peptide uORFs, Hayden and Jorgensen created “uORF-Finder”, a Perl program that compares the mORF amino acid sequence of cDNAs from one collection with the mORF sequences of another species' collection to identify putative mORF homologs, and then compares uORFs in the 5′ UTRs of the two paired sequences to identify uORFs with conserved amino acid sequences (Hayden and Jorgensen, 2007. BMC Biology 5:32). By comparing full-length cDNA sequences from Arabidopsis and rice, distinct homology groups of conserved peptide uORFs are so identified. Skarshewski et al. describe the use of “uPEPperoni”, an online tool for upstream open reading frame location and analysis of transcript conservation (Skarshewski, A., et al. 2014. BMC Bioinform. 15: 36. doi: 10.1186/1471-2105-15-36).

Rather than making use of bioinformatics-based analysis, Ingolia et al. describe methods for ribosome profiling: identifying uORFs by evaluating ribosome occupancy of upstream open reading frames and other sequences. See, for example, U.S. Pat. No. 9,677,068; Ingolia N. T., 2014. Cell Reports 8: 5, 1365-1379. See also Ingolia N. T. 2011. Cell 11; 147: 789-802 in which the authors describe how the majority of putative lincRNAs contain regions of high translation comparable to protein-coding genes. Specific start sites marked by harringtonine followed by ribosome footprints extended to the first in-frame stop codon. The majority of novel near-cognate initiation sites detected drive the translation of uORFs. This is consistent with the high level of translation that is observed on many 5′ UTRs as opposed to 3′ UTRs, which are almost devoid of ribosomes.

In contrast to prior described methods, the new methodology of the current invention identifies uORFs based the ability to sharply delineate stop codons based on an abrupt drop off (i.e., a precipitous decline in) ribosome occupancy at those locations, as opposed to identification of start codons, which are often non-canonical and less readily defined.

The present invention relates to methods and compositions for identifying and characterizing uORFs in eukaryotes, and specifically plants, and means for modifying the uORFs to produce desirable traits. In so doing, means for producing commercially valuable plants and crops as well as the methods for making them and using them are identified.

The uORFs identified and characterized with the present methods may be modified for the purpose of producing plants with modified traits, particularly traits that address agricultural, food-production and material-production needs as well as needs for environmental rehabilitation and carbon sequestration. These traits may provide significant value in that they allow the plant to thrive in hostile environments, where, for example, temperature, water and nutrient availability or salinity may limit or prevent growth of plants lacking the modified traits. The traits may also comprise desirable morphological alterations, including alterations of flowering time, larger or smaller size, disease and pest resistance, light response, alterations in biochemical composition, and other desirable phenotypes. In particular, with growing interest in producing crops under controlled indoor conditions, traits such as delayed flowering or more compact architecture are often desirable, particularly in leafy greens.

The present invention also relates to methods and compositions for eliminating undesirable plants, for example, weeds, in cultivated beds or fields of crop or ornamental plants, lawns, playing fields, or in municipal settings.

Other aspects and embodiments of the invention are described below and can be derived from the teachings of this disclosure as a whole.

### SUMMARY OF THE INVENTION

The present description pertains to novel methods for identification of regulatory regions within the genome of a eukaryotic organism comprising one or more upstream open reading frames (uORFs) that reside upstream of one or more downstream open reading frames that encoding one or more polypeptides including regulatory polypeptides or transcription factors. Once identified, the uORF sequences can be modified through gene editing techniques to induce new desired phenotypes in a cell (i.e., a target cell) or organism.

In one embodiment, the present description pertains to a method for identifying a uORF through application of an algorithm to ribosome profiling data. Rather than a conventional but often unsuccessful approach to finding ORF sequences by looking for at a canonical ATG start codon or even an alternative start codon, with or without ribosome enrichment information, the present algorithm and unconventional method identify the presence of the uORF in the genome of an organism based on the existence of ribosome enrichment in the interval from one stop codon to the next stop codon within the same open reading frame. The latter stop codon represents the end of a putative uORF. The sequence immediately upstream of the latter stop codon represents a potential target for gene editing that disrupts the function of the uORF. Once the uORF function is disrupted, translation of the downstream main ORF is increased and the polypeptide encoded by the main ORF produces an improved trait, that is, a desirable phenotype, in an organism or a target cell of the organism.

The present method identifies putative uORFs through application of an algorithm that evaluates ribosome profiling data and includes the steps of:
a) identifying an existent or putative major Open Reading Frame (ORF) in the genome of an organism and obtaining ribosome profiling data in the genome of the organism. Identification of the ORF may be through original research (that is, de novo) or from extant public or private knowledge of functional or putatively functional gene sequences;
b) evaluating the ribosome occupancy of at least one region of the genome that is upstream of the ORF;
c) identifying a location of the genome upstream of the ORF where there is both ribosome enrichment, and downstream of the that location there is an abrupt drop-off in ribosome occupancy;
d) identifying the stop codon of a putative or actual uORF in the genome from the appearance of an abrupt drop off in ribosome occupancy at the location;
e) identifying a prior or “first” stop codon upstream and in frame of the putative uORF's stop codon; and
f) thus, the algorithm identifies presence of a putative uORF within the genome from the ribosome enrichment data within the interval from the first stop codon to the putative uORF's stop codon within the same open reading frame.

The present description is also directed to a cell, plant cell, plant, or other organism that comprises an introduced targeted genetic modification at a native genomic locus. The native genomic locus comprises a mutation in a uORF that is located in the 5′ UTR of a gene that encodes a polypeptide with cellular regulatory activity. The polypeptide comprises an amino acid sequence with a percentage identity to a polypeptide provided in the Sequence Listing with this application, wherein the percentage identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to a polypeptide provided in the instant Sequence Listing. The targeted genetic modification increases expression level and/or activity of the encoded polypeptide with cellular regulatory activity.

The present description also pertains to a crop, turf, weed, or ornamental plant that contains an introduced targeted genetic modification. The introduced targeted genetic modification comprises a non-native allele that further comprises a mutation within a uORF located in the 5′ UTR of a gene that encodes a polypeptide with cellular regulatory activity. The polypeptide has an amino acid sequence identity to a sequence provided in the Sequence Listing provided with this application. The Sequence Listing identifies loci that encode polypeptides of interest which are subject to upstream uORF control, along with the identified position and sequence of the uORFs in a reference plant genome (Arabidopsis). The presence of uORFs upstream of an mORF encoding a homologous polypeptide in a target crops will typically be conserved. The genetically modified plant exhibits an improved trait compared to a reference or control plant of the same species that lacks the non-native allele. The amino acid sequence identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to a sequence provided in the Sequence Listing.

The present description is also directed to a method for producing an improved trait in a crop plant comprising introducing a targeted genetic modification into the genome of the crop plant. The targeted genetic modification creates a non-native allele of a gene that further comprises a mutation in a uORF in the 5′UTR of a gene that encodes a polypeptide with cellular regulatory activity. The polypeptide has an amino acid sequence with a percentage identity to a polypeptide provided in the Sequence Listing filed with this description. A plant of the crop plant is then selected and the selected plant contains the non-native allele and exhibits the improved trait compared to a reference or control plant of the same species that lacks the non-native allele. The targeted genetic modification modulates the expression level and/or activity of the encoded polypeptide with transcriptional regulatory activity; and the percentage identity is at least 30%, 35%, 40%, 45%, 50%, 55%, 60%, 65%, 70%, 75%, 80%, 85%, 90%, 95%, 96%, 97%, 98%, 99%, or about 100%.

The instant description is also directed to a process of killing the cells of plant involving the mutation of a uORF that is upstream of a main ORF that encodes a necrosis-inducing polypeptide that triggers death of the cells of the plant. In this method, parts of the plant are contacted with a suspension containing cells of an Agrobacterium strain containing a nucleic acid construct. The nucleic acid construct may also be delivered to the plant by other mechanisms including coating on nanoparticles (such as, but not limited to, DNA nanoparticles, carbon nanotubes, carborundum powder, magnetofection, peptide nanoparticles and clay nanosheets). Various methods of delivery of nucleic acid constructs into plant cells are detailed by Lv et al., 2020, Plant Journal, Volume 104, 880-891 (doi.org/10.1111/tpj.14973). The nucleic acid construct comprises a gene editing system that expresses in the cells of the plant a guide RNA that introduces a mutation in a uORF that is upstream of a main ORF in the genome of the plant.

The instant description is also directed to an herbicidal composition that is contacted to a plant such as a weed, wherein the genome of the plant comprises a main ORF that encodes a necrosis-inducing polypeptide that triggers death of the cells of the plant. The herbicidal composition comprises a suspension containing cells of an Agrobacterium strain containing a nucleic acid construct that comprises a gene editing system. The gene editing system expresses a guide RNA in the cells of a target weed and the guide RNA introduces a mutation in a uORF that is upstream of a main ORF in the genome of the plant or weed.

The instant description is also directed to a genetically modified cell comprising a non-naturally occurring polynucleotide that has been produced by gene editing. The non-naturally occurring polynucleotide encodes for a polypeptide that results in the production of an increased level of a target molecule or enzyme compared to a control microorganism that does not include the non-naturally polynucleotide. The non-naturally occurring polynucleotide comprises a mutation in a uORF that resides in the same transcript as a main ORF that encodes the polypeptide.

The instant description also pertains to a process for controlling cancerous cells or cells of a tumor, the method comprising contacting the cancerous cells or cells of the tumor with a delivery vector containing a nucleic acid construct comprising a gene editing system which expresses in the cells a guide RNA which introduces a mutation in a uORF that is upstream of a main ORF in the genome of said cells. The main ORF encodes polypeptide that triggers death or inhibits cell division of the cancerous cells or cells of the tumor.

The instant description also pertains to a process for improving plant traits through exogenous application of the short peptides (so-called “uPEPs”) that are encoded by uORFs. Through such a process, a uPEPs is used as biostimulant to enhance crop growth, yield, quality, harvestability, and/or performance.

### BRIEF DESCRIPTION OF THE SEQUENCE LISTING AND DRAWINGS

The Sequence Listing provides exemplary polynucleotide and polypeptide sequences of the instant disclosure. The Sequence Listing is named UOR-0002P_ST25, was created on May 27, 2022 and is 6,751,561 bytes in size. The entire content of the Sequence Listing is hereby incorporated by reference.

FIGS. 1 through 3 show graphical representations of ribosome profiling and analysis. The black trace represents the ribosome coverage along the length of the cDNA sequence. Long ORF (mORF) is a solid black line above the trace, with the AUG annotated as a black triangle at coverage=0. All ribosome profile ratios (crosses (X)) and ribosome profile differences (plus (+)) are shown above the coverage=0 (and scaled to fit the maximum ribosome profile count). All possible AUG codons are black circles at coverage=0. The stop-stop intervals>50 are shown as dashed, dotted and dot-dash horizontal lines (represent the three reading frames) below coverage=0 (note the long stop-stop corresponding to the long ORF here is a dotted line extends upstream of the ATG start codon). Stop-stop fragment selected with ratios (crosses (X)) and/or differences (plus (+)) as being statistically significant are annotated at the 3′ stop.

FIG. 4 depicts a binary vector that may be delivered into the cells of a plant by means of Agrobacterium or via other methods such as the use of nanoparticles. The genes bounded by T-DNA borders comprise a gene editing system that will express a guide RNA (encoded by the DNA sequence denoted “guide”) in the cells of the plant targets the knock-out of, or reduces the activity of, a uORF that has a native role in suppressing the expression of a mORF encoding a polypeptide that causes a trait of interest. The transformation selection typically encodes resistance to an herbicide or antibiotic that enables transformed edited cells to be selected and regenerated into a whole plant. With the system expressed, the polypeptide is upregulated in the cells of the plant and the desired trait is obtained. In one embodiment of the inventions the plant in question is a weed plant and the T-DNA does not necessarily integrate into the host weed genome, but the gene editing system transiently expresses a guide RNA that knocks out or reduces the activity of a uORF that natively suppresses a polypeptide that triggers cellular necrosis. When the system is activated in cells of a target weed, necrosis is induced and the weed is killed or controlled.

FIGS. 5 and 6 show the relative ribosome profile coverage (ribosome coverage/RNA-Seq coverage) 100 nucleotides either side of the AUG start (FIG. 5) and stop (FIG. 6) codon for all Arabidopsis genes with a leader sequence greater than 200 nucleotides and or tail sequence longer than 200 nucleotides. Histograms show the counts of the relative expression (ribosome coverage/RNA-Seq coverage) are in five regions relative to the AUG start in FIG. 5 (1=−70 to −42, 2=−41 to −14, 3=−13 to 14, 4-15 to 42 and 5=43 to 70) and to the stop codon in FIG. 6 (1=−70 to −37, 2=−36 to −4, 3=−3 to 14, 4-15 to 42 and 5=43 to 70).

FIG. 7 shows Amaranthus hybridus subsp. hybridus (hybridus contigs scaffolded to hypochondriacus): polished genome contigs of Amaranthus hybridus scaffolded to pseudochromosomes of Amaranthus hypochondriacus with reveal finish (v1.0, id57429). Gray bars present AUG codon \for each of the three reading frames (noting the gene is in reverse order) and black bars represented stop codons (UAG, UAA and UGA) for each of the three reading frames (noting the gene is in reverse order). Potentially uORFs are defined by two adjacent stop-stop intervals in the same open reading frame. In this way, putative uORF of genes can be identified and tested as candidates for gene editing.

FIG. 8 illustrates a method for optimizing activity from a transgene by dampening the translation of an encoded protein by inclusion of a uORF in the transcript. In the case where a transgenic event has already been generated in a target plant, the uORF can be inserted by gene editing. Alternatively, the uORF can be engineered into the transformation construct prior to the transformation process. In this latter case, the uORF would preferably be introduced into the leader of the transgene to be overexpressed through direct synthesis, or ligation, at the time when the transformation construct is assembled. Footnote 1, indicated by the italicized numeral “1” in FIG. 8, shows mRNA from transgene; introduced uORF dampens translation of mORF. Footnote 2, indicated by the italicized numeral “2” in FIG. 8, shows a uORF intentionally introduced into this region (by gene editing) to dampen activity of transgene by reducing translation of encoded mRNA into protein.

### DETAILED DESCRIPTION

### Definitions

“uORFs” are upstream open reading frames, that often reside in an mRNA transcript located upstream of protein-coding main ORFs (Note that mORFs, which are also sometimes referred to as long ORFs or major ORFs and the terms mORF, main ORF, long ORF and major ORF are used interchangeably in this application). uORFs are a class of small ORFs that acts as repressors of their downstream mORFs. uORFs sometimes encode evolutionarily conserved functional peptides such as cis-acting regulatory peptides and which act as repressors, including for example, through translational repression.

A “polypeptide” is an amino acid sequence comprising a plurality of consecutive polymerized amino acid residues e.g., at least about 15 consecutive polymerized amino acid residues, optionally at least about 30 consecutive polymerized amino acid residues, at least about 50 consecutive polymerized amino acid residues. In many instances, a polypeptide comprises a polymerized amino acid residue sequence that is a transcription factor or a domain or portion or fragment thereof. Additionally, the polypeptide may comprise 1) a localization domain, 2) an activation domain, 3) a repression domain, 4) an oligomerization domain, or 5) a DNA-binding domain, or the like. The polypeptide optionally comprises modified amino acid residues, naturally occurring amino acid residues not encoded by a codon, non-naturally occurring amino acid residues.

“Identity” or “similarity” refers to sequence similarity between two polynucleotide sequences or between two polypeptide sequences, with identity being a stricter comparison. The phrases “percent identity” and “% identity” refer to the percentage of sequence identity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value therebetween. Identity or similarity can be determined by comparing a position in each sequence that may be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences.

The term “homolog” or “homologue” as further described and used herein means a polypeptide or transcription factor from the same species or a different species which has a substantial level of identity within either its conserved domain and/or across its entire sequence, wherein the level of identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity as compared to a first polypeptide or transcription factor and which polypeptide or transcription factor has a similar or comparable function in a cell or organism as compared to the first polypeptide or transcription factor.

“Orthologs” are evolutionarily related genes that have similar sequence and similar functions. Orthologs are structurally related genes in different species that are derived by a speciation event.

The term “introduced targeted genetic modification” or “targeted genetic modification” refers to a change in the DNA sequence of a plant at a specific chromosomal position (also known as a locus) in the genome which is chosen by a skilled practitioner (such as plant breeder or molecular biologist) and which change is introduced by a process of gene editing and/or selection using a specific complementary nucleic acid molecule sequence as a guide or probe to enable the process.

The term “native genomic locus” refers to a gene or DNA sequence that is present in the genome of a wild-type plant at particular chromosomal position of a given species. The “native genomic locus” typically comprises a region spanning a start to stop codon, along with any intervening introns, that is transcribed to generate a main ORF that encodes a long polypeptide that is typically around 100 amino acids or more in length, as well as the associated upstream regulatory elements including the promoter region and any elements that control the activity of the mORF such as uORFs. A uORF is present in the same mRNA transcript as the mORF that the uORF regulates; both the uORF and the mORF can therefore be considered part of the same overall native genomic locus. A native genomic locus is often specified by reference to an accession number, deposited in GenBank, which, for example, indicates the DNA sequence and encoded polypeptide that is present at that position. It should also be noted that a locus may encode multiple protein variants that result from alternative splicing of mRNA and these variants are represent by different “gene models” that are denoted by the accession number followed by a dot and a number.

The terms “non-native allele of a gene” or “non-naturally occurring allele of a gene” refer to a sequence variant of a gene (where the term “gene” potentially includes both the protein coding region, encoded by a main ORF, as well as upstream control elements such as the promoter region and elements such as uORFs) from a given plant species that has a sequence of nucleotides which has been produced by human intervention (e.g., through gene editing or selection such as through TILLING) and which is not typically found in nature in either the genome of a wild-type plant of that species or in the genome of a plant of that species taken from a naturally-occurring wild population. The term “TILLING” is an acronym for “targeted induced local lesions in genome” and has been reviewed by Kurowska et al., 2011, Appl Genet. 52(4): 371-390.

The term “variant”, as used herein, may refer to polynucleotides or polypeptides, that differ from the presently disclosed polynucleotides or polypeptides, respectively, in sequence from each other, and as set forth below.

With regard to polynucleotide variants, differences between presently disclosed polynucleotides and polynucleotide variants are limited so that the nucleotide sequences of the former and the latter are closely similar overall and, in many regions, identical. Due to the degeneracy of the genetic code, differences between the former and latter nucleotide sequences may be silent (i.e., the amino acids encoded by the polynucleotide are the same, and the variant polynucleotide sequence encodes the same amino acid sequence as the presently disclosed polynucleotide. Variant nucleotide sequences may encode different amino acid sequences, in which case such nucleotide differences will result in amino acid substitutions, additions, deletions, insertions, truncations or fusions with respect to the similar disclosed polynucleotide sequences. These variations result in polynucleotide variants encoding polypeptides that share at least one functional characteristic. The degeneracy of the genetic code also dictates that many different variant polynucleotides can encode identical and/or substantially similar polypeptides in addition to those sequences illustrated in the Sequence Listing.

Also within the scope of the invention is a variant of a nucleic acid listed in the Sequence Listing, that is, one having a sequence that differs from the one of the polynucleotide sequences in the Sequence Listing, or a complementary sequence, that encodes a functionally equivalent polypeptide (i.e., a polypeptide having some degree of equivalent or similar biological activity) but differs in sequence from the sequence in the Sequence Listing, due to degeneracy in the genetic code. Included within this definition are polymorphisms that may or may not be readily detectable using a particular oligonucleotide probe of the polynucleotide encoding polypeptide, and improper or unexpected hybridization to allelic variants, with a locus other than the normal chromosomal locus for the polynucleotide sequence encoding polypeptide.

The term “plant” includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae. See for example, Daly et al. (2001) Plant Physiol. 127: 1328-1333; Ku et al. (2000) Proc. Natl. Acad. Sci. 97: 9121-9126; and see also Tudge, in The Variety of Life, Oxford University Press, New York, NY (2000) pp. 547-606.

A “trait” is sometimes used interchangeably with the term “phenotype” and refers to a physiological, morphological, biochemical, or physical characteristic of a cell or organism, including of a plant or of a particular plant material or of a plant cell. In some instances, this characteristic is visible to the human eye, such as seed or plant size, or pigmentation, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, RNA Seq or reporter gene expression systems, or by agricultural observations such as stress tolerance, yield, or pathogen tolerance. Any technique can be used to measure the amount of, comparative level of, or difference in any selected chemical compound or macromolecule in the transgenic plants, however.

“Trait modification” refers to a detectable difference in a characteristic in a plant ectopically expressing a polynucleotide or polypeptide of the present invention relative to a plant not doing so, such as a wild-type plant. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, at least about an 85%, or about a 100%, or an even greater difference compared with a wild-type plant. It is known that there can be a natural variation in the modified trait. Therefore, the trait modification observed entails a change of the normal distribution of the trait in the plants compared with the distribution observed in wild-type plant.

“Wild type” or “Wild-type”, as used herein, refers to a cell, tissue or plant that has not been genetically modified to mutate, knock out, ectopically-express, or overexpress one or more of the presently disclosed target genes (such as genes encoding transcription factors). Wild-type cells, tissue or plants may be used as controls to compare levels of expression and the extent and nature of trait modification with cells, tissue or plants in which target gene expression is altered or ectopically expressed, e.g., in that it has been knocked out or overexpressed.

“Yield” or “plant yield” refers to increased plant growth, increased crop growth, increased biomass, and/or increased plant product production, and is dependent to some extent on temperature, plant size, organ size, planting density, light, water and nutrient availability, and how the plant copes with various stresses, such as through temperature acclimation and water or nutrient use efficiency.

A “crop” plant includes cultivated plants or agricultural produce, and may be a grain, vegetables, or fruit plant, generally considered as a group. A crop plant may be grown in commercially useful numbers or amounts.

An “Improved Trait” that may be conferred to plants and provide an environmental, commercial, or ornamental advantage to crop plants may include, but is not limited to, a trait selected from the group consisting of:
a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level.
“uPEP” as used herein means the small peptide encoded by a uORF.

### DESCRIPTION OF THE SPECIFIC EMBODIMENTS

Upstream open reading frames (uORF) are short open reading frames that could potentially code for peptides and which reside within the leader sequence of a messenger RNA. To avoid confusion, within this description the term ‘leader’ sequence is often used rather than five prime untranslated regions (5′UTRs). This distinction is made because, by definition, uORF implies translation, and so the name ‘untranslated’ may be misleading. Similarly, the three prime untranslated regions (3′UTR) may also be capable of translation and so in this description the ‘tail’ sequence may sometimes be used to refer to this region. The present description relates to novel methods for identification of regulatory regions within the genome of a eukaryotic organism comprising one or more uORFs encoding one or more polypeptides including regulatory polypeptides or transcription factors. Once identified, the uORF sequences can be modified through gene editing techniques to induce new desired phenotypes in the target cell or organism.

The Challenge of Annotating uORFs

The small size of upstream open reading frames makes ab initio annotation extremely challenging. This is because, in even small eukaryotic genomes, there is a high statistical likelihood of finding an open frame of a hundred amino acids or 300 nucleotides or less, purely by chance. It is therefore difficult to discriminate short open reading frames that are functional from those that exist by chance alone. For this reason, most gene prediction tools only consider open reading frames greater than 100 amino acids. The exception to this is when shorter amino acids have been determined through experimental evidence, through homology to known genes of short amino acid sequence or other related short sequences. Small peptides of less than 100 aa remain an under-represented in almost all genome annotation (Hellens 2016. Trend Plant Sci. 21:317-328).

uORF annotation is made more complicated as there is increasing evidence that these short open reading frames do not follow that normal convention of most annotated peptides by starting with an AUG codon and a methionine amino acid. A number of well documented uORF sequences, including the uORF in GDP galactose pyrophosphorylase (GGP), has been shown to start with a non-AUG (also referred to as near cognate or non-canonical) start codon.

Taken together, these two features of uORFs, namely the small size and noncanonical start codon, make annotation prediction particularly difficult using computational means alone.

Using Data and a Novel Approach to Predict and Annotate uORFs

Ribosome profiling is a technique that uses next-generation sequencing technologies to display the region that ribosomes reside on a messenger RNA molecule. While the footprint does not demonstrate translation, it does demonstrate ribosome occupancy, and translation may therefore be implied. This information has been essential in the annotation of upstream open reading frames as the peptide sequence themselves are very rarely seen in accurate-mass-based peptide detection methodologies. Indeed, for most upstream open reading frames, ribosome profiling along with mutational analysis is the only evidence available to demonstrate functional uORFs.

Many methods have used ribosome profiling data to guide the annotation of uORFs, however, all methods to date rely on determining potential uORF start and stop sites and then looking for ribosome enrichment along the candidate uORF. Whilst the majority of these approaches have assumed an ATG start, more recent modifications have extended the potential start sites to include all the possible near cognate start sites (where one of the nucleotides A, U or G is replaced with a different nucleotide). Thus, detecting uORFs by looking for their translational start site is challenging because start codons other than AUG are frequently used. In addition, ribosomes accumulate in the leader sequence prior to translation initiation. By contrast, ribosome profiling data accurately maps translation stop sites. The three stop codons: UAA, UAG and UGA appeared to be ubiquitously used in both long open reading frames and shorter upstream open reading frames. Therefore, by using ribosome profiling data to predict stop codons the corresponding sequence interval between two in-frame stop codons can be assumed to contain the upstream open reading frame.

Our novel approach, which is the basis of the invention detailed herein, does not make any assumption about the position where a uORF starts. Rather, an open reading frame interval from one stop codon to the next stop codon within the same open reading frame is determined and it is assumed that if there is ribosome enrichment within this region, then the start of the uORF exists downstream of the first stop codon (5′ stop)). The ability to identify these stop-stop intervals and the use of the stop-stop interval to denote the presence of a uORF is are the novelty within this methodology. By determining the boundaries of the region within which a uORF exists in this manner, it is then possible to target the region through mutation and/or gene editing to modify or remove the uORF. The inventive approach detailed herein has been reduced to practice through its application to a variety of datasets including datasets from Arabidopsis.

### Raw Data Preparation

Raw data was downloaded and trimmed according to the publication method for each dataset, using the Trim Sequence (1.0.2) tools in the Galaxy environment.

Gene sequences for 5′UTR, 3′UTR and cDNA were downloaded from the TAIR (website using TAIR10 (20101214 updated) annotation files.

In Galaxy, BWA (0.7.17.4) was used to map the ribosome profiling short reads to the Arabidopsis gene model. BAM filter was used to remove unmapped reads. BED genome coverage (2.29.2 and −dz coverage output) was then used to generate file BEDgraph file for downloading. Files were imported as .csv into the R package for further analysis.

### Ribosome Profiles at Starts and Stops of Long Open Reading Frames

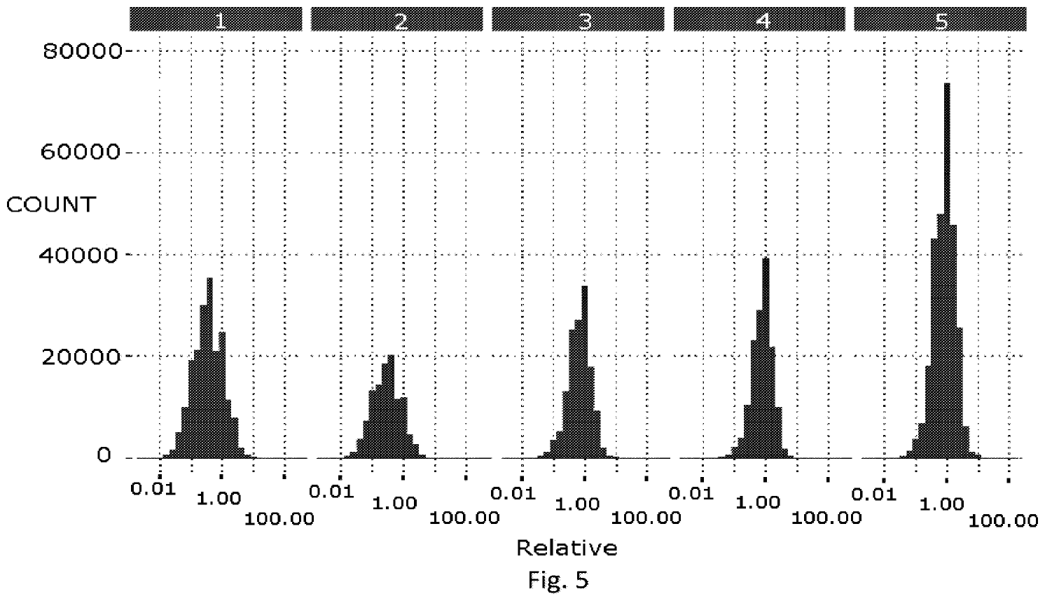

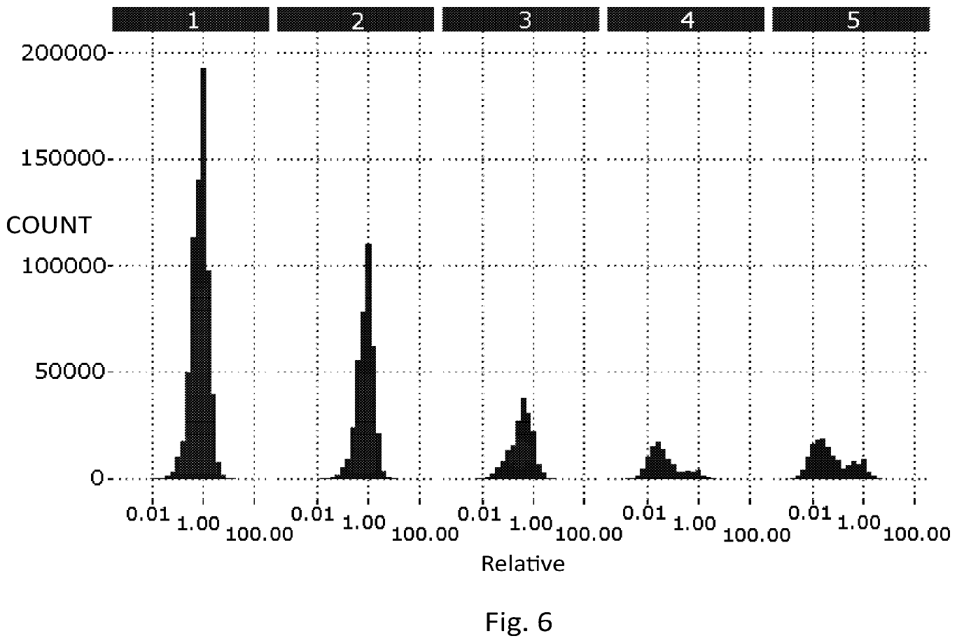

To determine the ribosome profiling at long ORFs, ribosome coverage was calculated for all annotated long open reading frames cDNA from the Arabidopsis genome. The ribosome profiles around the long ORF start and stop codons were generated. Taking a window of 100 nucleotides before and after both start and stop, the ribosome profiling coverage for all the Arabidopsis genes was determined. Coverage for both ribosome profiling data and RNA-Seq data was generated using the Liu datasets (Liu et al. 2013. Plant Cell 25: 3699-3710). The relative ribosome profiling value (the ribosome profile coverage divided by the RNAseq coverage, was determined for each nucleotide position along the sequence before and after the start and stop codon. FIGS. 5 and 6 show a graphical representation of these data.

Given that ribosome profiling shows a distinct difference at the stop codon, these observations are used to support the annotation of novel upstream open reading frames. Because ribosome profiling was less accurate at predicting start codon, potential regions where uORFs may reside were identified by identifying all stop-stop intervals within the mRNA sequence of a given gene.
Code 1 identifies all stop-stop intervals where k<−50 #sets ORF min to 50 bases, start_codons<-c(“TGA”,“TAA”,“TAG”) and stop_codons<-c(“TGA”,“TAA”,“TAG”) and s2 is the sequence for the gene of interest (GOI) under consideration.

```
#find all ORF start
start_pos <- c( )
for (codon in start_codons) {
  matches <- matchPattern(codon,s2)
  start_pos <- c(start_pos, start(matches))
  }
start_pos <- sort(start_pos)
#find all ORF stops
stop_pos <- c( )
for (codon in stop_codons) {
  matches <- matchPattern(codon,s2)
  stop_pos <- c(stop_pos, start(matches))
  }
stop_pos <- sort(stop_pos)
```

Code 2. Identified the longest stop-stop regions using code modified (highlighted in bold) from www.montefiore.ulg.ac.be/˜kbessonov/archived_data/GBIO009-1course2012/presentations/HW1_2_review_slides_ORF_Finder.pdf

#ORF set-up
rm(for_plotting)
for plotting <- data.frame(“count” = 0, “frame” = 0, “current_start” = 0, “length” = 0)
stop_pointers <- c(0,0,0)
count <- 0
#find Stop for each Start according to reading frame from start = start_pos and stop =
stop_pos

| | |
|---|---|
| for (current_start in start_pos) { | #loop current_start from 1st to last |

start_pos
frame <- (current_start%%3) + 1
= the remainder of (current_start/3) + 1 ie the reading frame

| | |
|---|---|
| stop_pointer <- stop_pointers[frame] | stop_pointer is one value and |

stop_pointers is 3, and
if (stop_pointer <= length(stop_pos) && (stop_pointer == 0

| | |
|---|---|
| ∥ stop_pos[stop_pointer] <= current_start)) { | # change < |

```
to <=
    stop_pointer <- stop_pointer + 1
    while ( (stop_pointer <= length(stop_pos))
          && ((stop_pos[stop_pointer] <= current_start)
            ∥ (((stop_pos[stop_pointer]%%3) + 1) != frame)) ) {
      stop_pointer <- stop_pointer + 1
    }
    stop_pointers[frame] <- stop_pointer
    if (stop_pointer <= length(stop_pos)) {
      if ((stop_pos[stop_pointer] + 2 − current_start + 1) > k ) {
        count <- count + 1
        length = (stop_pos[stop_pointer]+2−current_start+1)
        for_plotting <- add_row(for_plotting, count,
              frame,
              current_start,
              length)
        current_stop <- current_start + length
```

Code 3. Calculates the ribosome profile counts over a span of NGS read length (read_length)+window at the end of the stop-stop fragments, taking into account the 15 nucleotides described above (offset<−15, window<−30, read_length<−29). Where GOI equals the gene of interest.

```
#add the RP_ratio calculator here
#Before the STOP
if (current_stop <= nchar(s2)-200) { ## to avoid 0 RP ratio at the 3′
        before_STOP_coverage <- filter(GOI_coverage,
              RP_fragment_start >= current_stop − (window +
read_length) &
              RP_fragment_start <= current_stop)
        before_STOP_coveage_total <- sum(before_STOP_coverage$Coverage)
        #Atfer the STOP
        after_STOP_coverage <- filter(GOI_coverage,
              RP_fragment_start >= current_stop + offset &
              RP_fragment_start <= current_stop + (offset + window +
read_length))
        after_STOP_coverage_total <- sum(after_STOP_coverage$Coverage)
        RP_ratio_of_coverage_at_STOP <-
(log(before_STOP_coveage_total)/log(after_STOP_coveage_total))
        RP_delta_of_coverage_at_STOP <- ifelse(before_STOP_coveage_total −
after_STOP_coveage_total >= 0, before_STOP_coveage_total −
after_STOP_coveage_total, 0)
        print(“RP Ratio”)
        print(RP_ratio_of_coverage_at_STOP)
        current_stop <- current_start + length
        RP_ratio_for_plotting <- add_row(RP_ratio_for_plotting,
          current_stop,
          window,
          offset,
          before_STOP_coveage_total,
          after_STOP_coveage_total,
          RP_ratio_of_coverage_at_STOP,
          RP_delta_of_coverage_at_STOP)
            }
        #}
        #end window loop
        }
      }
    }
  }
```

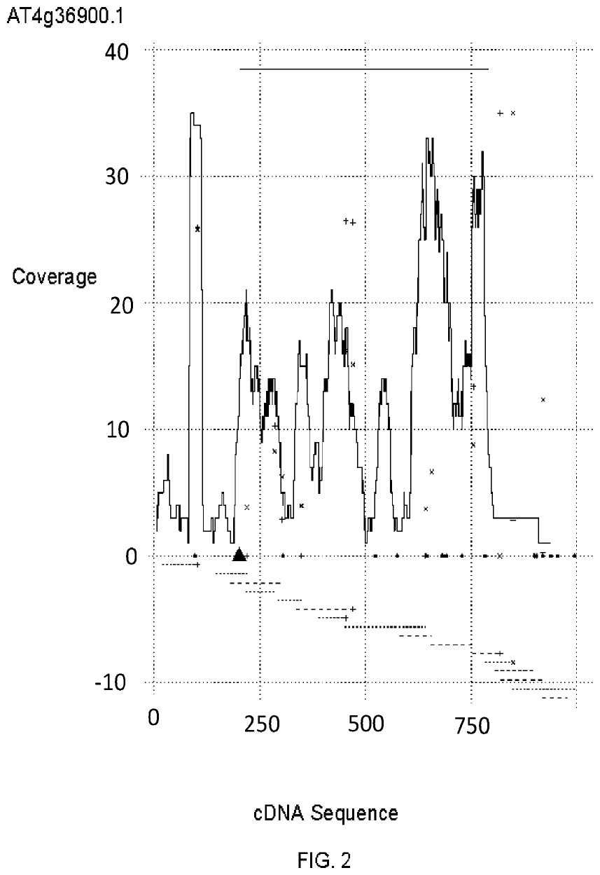

The output of this analysis is summarized in FIG. 2 where the gene of interest is AT4G26850.1 (the VTC2 gene, GDP galactose pyrophosphorylase AT4G26850.1)

### Whole-Genome Analysis of Ribosome Profiling Stop-Stop Fragments

Codes 1 to 3 were repeated for each of the 41,671 cDNAs in the TAIR annotations. Stop-Stop fragments were filtered for ratios that were five-fold difference between the before and after the stop, where the after stop count was greater than one. For each of the three datasets used, box plot analysis of the stop-stop coverage data was used to determine quartile 1 (Q1) ribosome profile count, and this value was used to determine the appropriate ribosome profiles difference (referred to as the Delta) for those gene models where ribosome counts after the stop codon=0, so ratios could not be calculated.

The Sequence Listing includes the culmination of genome analysis of ribosome profiles for all stop-stop regions for each Arabidopsis gene annotation using three datasets (Liu et al. 2013. Plant Cell 25: 3699-3710; Hsu et al. 2016. PNAS 113: E7126-E7135; and Bazin et al. 2017. PNAS 114: E10018-E10027). Statistically significant stop-stop fragments count before and after the fragment and for the leader, long ORF and tail regions are reported. For the fragments in the leader region, these are separated into the total number of short uORF, the number there are unique (noting some leader sequences may have multiple short uORF) and also the mORF count (noting by the definition provided here all long open reading frames will be extended to the upstream stop codon the proceeds the initiating AUG). For the unique uORF count (excluding multiple uORF within the leader sequence) the number detected using the ratio calculation, the delta calculation (when the ribosome count after the stop codon equal zero) or both are summarized. Finally, data lists for each of the three datasets were compared and only candidate uORF that appeared in at least two of the three datasets were included in the final list of 1999 gene models with potential uORF. Within the Sequence Listing the identified Arabidopsis sequences comprising uORFs are provided as the odd numbered sequences from SEQ ID NO: 1-3997, whereas the corresponding predicted PEP in each case is provided as the subsequent even numbered sequence, i.e., SEQ ID NO: 2, 4, 6 . . . 3998. The polypeptide products of the main ORFs from the loci identified as containing upstream uORFs are provided in SEQ ID NO: 3999-5155. Finally, example non-Arabidopsis DNA sequences comprising uORFs are provided in SEQ ID NO: 5156-5227.

Descriptions of the sequences that appear in the Sequence Listing are presented in Table 1.

TABLE 1
Description of sequences in the Sequence Listing

| SEQ | |
|---|---|
| ID NO: | Sequence Description |
| 1 | AGI number: AT1G01030.1 uORF ′5 STOP: 416 uORF 3′ STOP: 467 leader length: 613 |
| 2 | AGI number: AT1G01030.1 uORF ′5 STOP: 416 uORF 3′ STOP: 467 leader length: 613 |
| 3 | AGI number: AT1G01060.2 uORF ′5 STOP: 169 uORF 3′ STOP: 256 leader length: 550 |
| 4 | AGI number: AT1G01060.2 uORF ′5 STOP: 169 uORF 3′ STOP: 256 leader length: 550 |
| 5 | AGI number: AT1G01060.4 uORF ′5 STOP: 138 uORF 3′ STOP: 255 leader length: 380 |
| 6 | AGI number: AT1G01060.4 uORF ′5 STOP: 138 uORF 3′ STOP: 255 leader length: 380 |
| 7 | AGI number: AT1G01060.4 uORF ′5 STOP: 161 uORF 3′ STOP: 248 leader length: 380 |
| 8 | AGI number: AT1G01060.4 uORF ′5 STOP: 161 uORF 3′ STOP: 248 leader length: 380 |
| 9 | AGI number: AT1G01060.5 uORF ′5 STOP: 252 uORF 3′ STOP: 336 leader length: 765 |
| 10 | AGI number: AT1G01060.5 uORF ′5 STOP: 252 uORF 3′ STOP: 336 leader length: 765 |
| 11 | AGI number: AT1G01060.5 uORF ′5 STOP: 229 uORF 3′ STOP: 310 leader length: 765 |
| 12 | AGI number: AT1G01060.5 uORF ′5 STOP: 229 uORF 3′ STOP: 310 leader length: 765 |
| 13 | AGI number: AT1G01060.5 uORF ′5 STOP: 677 uORF 3′ STOP: 728 leader length: 765 |
| 14 | AGI number: AT1G01060.5 uORF ′5 STOP: 677 uORF 3′ STOP: 728 leader length: 765 |
| 15 | AGI number: AT1G01060.5 uORF ′5 STOP: 260 uORF 3′ STOP: 347 leader length: 765 |
| 16 | AGI number: AT1G01060.5 uORF ′5 STOP: 260 uORF 3′ STOP: 347 leader length: 765 |
| 17 | AGI number: AT1G01160.2 uORF ′5 STOP: 115 uORF 3′ STOP: 286 leader length: 226 |
| 18 | AGI number: AT1G01160.2 uORF ′5 STOP: 115 uORF 3′ STOP: 286 leader length: 226 |
| 19 | AGI number: AT1G01340.1 uORF ′5 STOP: 7 uORF 3′ STOP: 118 leader length: 204 |
| 20 | AGI number: AT1G01340.1 uORF ′5 STOP: 7 uORF 3′ STOP: 118 leader length: 204 |
| 21 | AGI number: AT1G01650.2 uORF ′5 STOP: 316 uORF 3′ STOP: 478 leader length: 676 |
| 22 | AGI number: AT1G01650.2 uORF ′5 STOP: 316 uORF 3′ STOP: 478 leader length: 676 |
| 23 | AGI number: AT1G01780.1 uORF ′5 STOP: 8 uORF 3′ STOP: 71 leader length: 158 |
| 24 | AGI number: AT1G01780.1 uORF ′5 STOP: 8 uORF 3′ STOP: 71 leader length: 158 |
| 25 | AGI number: AT1G01880.1 uORF ′5 STOP: 57 uORF 3′ STOP: 153 leader length: 118 |
| 26 | AGI number: AT1G01880.1 uORF ′5 STOP: 57 uORF 3′ STOP: 153 leader length: 118 |
| 27 | AGI number: AT1G02010.1 uORF ′5 STOP: 98 uORF 3′ STOP: 284 leader length: 155 |
| 28 | AGI number: AT1G02010.1 uORF ′5 STOP: 98 uORF 3′ STOP: 284 leader length: 155 |
| 29 | AGI number: AT1G02010.2 uORF ′5 STOP: 15 uORF 3′ STOP: 201 leader length: 72 |
| 30 | AGI number: AT1G02010.2 uORF ′5 STOP: 15 uORF 3′ STOP: 201 leader length: 72 |
| 31 | AGI number: AT1G02145.2 uORF ′5 STOP: 75 uORF 3′ STOP: 132 leader length: 228 |
| 32 | AGI number: AT1G02145.2 uORF ′5 STOP: 75 uORF 3′ STOP: 132 leader length: 228 |
| 33 | AGI number: AT1G02145.2 uORF ′5 STOP: 95 uORF 3′ STOP: 161 leader length: 228 |
| 34 | AGI number: AT1G02145.2 uORF ′5 STOP: 95 uORF 3′ STOP: 161 leader length: 228 |
| 35 | AGI number: AT1G02145.4 uORF ′5 STOP: 75 uORF 3′ STOP: 132 leader length: 228 |
| 36 | AGI number: AT1G02145.4 uORF ′5 STOP: 75 uORF 3′ STOP: 132 leader length: 228 |
| 37 | AGI number: AT1G02145.4 uORF ′5 STOP: 95 uORF 3′ STOP: 161 leader length: 228 |
| 38 | AGI number: AT1G02145.4 uORF ′5 STOP: 95 uORF 3′ STOP: 161 leader length: 228 |
| 39 | AGI number: AT1G02205.1 uORF ′5 STOP: 19 uORF 3′ STOP: 592 leader length: 688 |
| 40 | AGI number: AT1G02205.1 uORF ′5 STOP: 19 uORF 3′ STOP: 592 leader length: 688 |
| 41 | AGI number: AT1G02205.1 uORF ′5 STOP: 423 uORF 3′ STOP: 558 leader length: 688 |
| 42 | AGI number: AT1G02205.1 uORF ′5 STOP: 423 uORF 3′ STOP: 558 leader length: 688 |
| 43 | AGI number: AT1G02260.1 uORF ′5 STOP: 76 uORF 3′ STOP: 163 leader length: 321 |
| 44 | AGI number: AT1G02260.1 uORF ′5 STOP: 76 uORF 3′ STOP: 163 leader length: 321 |
| 45 | AGI number: AT1G02260.1 uORF ′5 STOP: 20 uORF 3′ STOP: 155 leader length: 321 |
| 46 | AGI number: AT1G02260.1 uORF ′5 STOP: 20 uORF 3′ STOP: 155 leader length: 321 |
| 17 | AGI number: AT1G02290.1 uORF ′5 STOP: 39 uORF 3′ STOP: 135 leader length: 244 |
| 48 | AGI number: AT1G02290.1 uORF ′5 STOP: 39 uORF 3′ STOP: 135 leader length: 244 |
| 49 | AGI number: AT1G02290.1 uORF ′5 STOP: 49 uORF 3′ STOP: 127 leader length: 244 |
| 50 | AGI number: AT1G02290.1 uORF ′5 STOP: 49 uORF 3′ STOP: 127 leader length: 244 |
| 51 | AGI number: AT1G02380.1 uORF ′5 STOP: 198 uORF 3′ STOP: 282 leader length: 304 |
| 52 | AGI number: AT1G02380.1 uORF ′5 STOP: 198 uORF 3′ STOP: 282 leader length: 304 |
| 53 | AGI number: AT1G02720.1 uORF ′5 STOP: 159 uORF 3′ STOP: 225 leader length: 289 |
| 54 | AGI number: AT1G02720.1 uORF ′5 STOP: 159 uORF 3′ STOP: 225 leader length: 289 |
| 55 | AGI number: AT1G02720.1 uORF ′5 STOP: 116 uORF 3′ STOP: 236 leader length: 289 |
| 56 | AGI number: AT1G02720.1 uORF ′5 STOP: 116 uORF 3′ STOP: 236 leader length: 289 |
| 57 | AGI number: AT1G02880.1 uORF ′5 STOP: 128 uORF 3′ STOP: 284 leader length: 456 |
| 58 | AGI number: AT1G02880.1 uORF ′5 STOP: 128 uORF 3′ STOP: 284 leader length: 456 |
| 59 | AGI number: AT1G02930.1 uORF ′5 STOP: 54 uORF 3′ STOP: 108 leader length: 236 |
| 60 | AGI number: AT1G02930.1 uORF ′5 STOP: 54 uORF 3′ STOP: 108 leader length: 236 |
| 61 | AGI number: AT1G03080.1 uORF ′5 STOP: 29 uORF 3′ STOP: 143 leader length: 351 |
| 62 | AGI number: AT1G03080.1 uORF ′5 STOP: 29 uORF 3′ STOP: 143 leader length: 351 |
| 63 | AGI number: AT1G03310.1 uORF ′5 STOP: 49 uORF 3′ STOP: 124 leader length: 157 |
| 64 | AGI number: AT1G03310.1 uORF ′5 STOP: 49 uORF 3′ STOP: 124 leader length: 157 |
| 65 | AGI number: AT1G03860.2 uORF ′5 STOP: 201 uORF 3′ STOP: 291 leader length: 386 |
| 66 | AGI number: AT1G03860.2 uORF ′5 STOP: 201 uORF 3′ STOP: 291 leader length: 386 |
| 67 | AGI number: AT1G03860.2 uORF ′5 STOP: 101 uORF 3′ STOP: 254 leader length: 386 |
| 68 | AGI number: AT1G03860.2 uORF ′5 STOP: 101 uORF 3′ STOP: 254 leader length: 386 |
| 69 | AGI number: AT1G03930.1 uORF ′5 STOP: 105 uORF 3′ STOP: 183 leader length: 670 |
| 70 | AGI number: AT1G03930.1 uORF ′5 STOP: 105 uORF 3′ STOP: 183 leader length: 670 |
| 71 | AGI number: AT1G03930.1 uORF ′5 STOP: 518 uORF 3′ STOP: 578 leader length: 670 |
| 72 | AGI number: AT1G03930.1 uORF ′5 STOP: 518 uORF 3′ STOP: 578 leader length: 670 |
| 73 | AGI number: AT1G03960.1 uORF ′5 STOP: 196 uORF 3′ STOP: 277 leader length: 370 |
| 74 | AGI number: AT1G03960.1 uORF ′5 STOP: 196 uORF 3′ STOP: 277 leader length: 370 |
| 75 | AGI number: AT1G03960.2 uORF ′5 STOP: 196 uORF 3′ STOP: 277 leader length: 942 |
| 76 | AGI number: AT1G03960.2 uORF ′5 STOP: 196 uORF 3′ STOP: 277 leader length: 942 |
| 77 | AGI number: AT1G03980.1 uORF ′5 STOP: 233 uORF 3′ STOP: 287 leader length: 237 |
| 78 | AGI number: AT1G03980.1 uORF ′5 STOP: 233 uORF 3′ STOP: 287 leader length: 237 |
| 79 | AGI number: AT1G04120.1 uORF ′5 STOP: 25 uORF 3′ STOP: 88 leader length: 338 |
| 80 | AGI number: AT1G04120.1 uORF ′5 STOP: 25 uORF 3′ STOP: 88 leader length: 338 |
| 81 | AGI number: AT1G04120.2 uORF ′5 STOP: 145 uORF 3′ STOP: 208 leader length: 458 |
| 82 | AGI number: AT1G04120.2 uORF ′5 STOP: 145 uORF 3′ STOP: 208 leader length: 458 |
| 83 | AGI number: AT1G04140.1 uORF ′5 STOP: 46 uORF 3′ STOP: 121 leader length: 309 |
| 84 | AGI number: AT1G04140.1 uORF ′5 STOP: 46 uORF 3′ STOP: 121 leader length: 309 |
| 85 | AGI number: AT1G04140.2 uORF ′5 STOP: 46 uORF 3′ STOP: 121 leader length: 309 |
| 86 | AGI number: AT1G04140.2 uORF ′5 STOP: 46 uORF 3′ STOP: 121 leader length: 309 |
| 87 | AGI number: AT1G04300.2 uORF ′5 STOP: 384 uORF 3′ STOP: 696 leader length: 630 |
| 88 | AGI number: AT1G04300.2 uORF ′5 STOP: 384 uORF 3′ STOP: 696 leader length: 630 |
| 89 | AGI number: AT1G04300.2 uORF ′5 STOP: 394 uORF 3′ STOP: 448 leader length: 630 |
| 90 | AGI number: AT1G04300.2 uORF ′5 STOP: 394 uORF 3′ STOP: 448 leader length: 630 |
| 91 | AGI number: AT1G04440.1 uORF ′5 STOP: 143 uORF 3′ STOP: 245 leader length: 560 |
| 92 | AGI number: AT1G04440.1 uORF ′5 STOP: 143 uORF 3′ STOP: 245 leader length: 560 |
| 93 | AGI number: AT1G04778.1 uORF ′5 STOP: 30 uORF 3′ STOP: 117 leader length: 55 |
| 94 | AGI number: AT1G04778.1 uORF ′5 STOP: 30 uORF 3′ STOP: 117 leader length: 55 |
| 95 | AGI number: AT1G04960.2 uORF ′5 STOP: 44 uORF 3′ STOP: 269 leader length: 444 |
| 96 | AGI number: AT1G04960.2 uORF ′5 STOP: 44 uORF 3′ STOP: 269 leader length: 444 |
| 97 | AGI number: AT1G04960.2 uORF ′5 STOP: 117 uORF 3′ STOP: 228 leader length: 444 |
| 98 | AGI number: AT1G04960.2 uORF ′5 STOP: 117 uORF 3′ STOP: 228 leader length: 444 |
| 99 | AGI number: AT1G05160.1 uORF ′5 STOP: 334 uORF 3′ STOP: 448 leader length: 343 |
| 100 | AGI number: AT1G05160.1 uORF ′5 STOP: 334 uORF 3′ STOP: 448 leader length: 343 |
| 101 | AGI number: AT1G05200.2 uORF ′5 STOP: 162 uORF 3′ STOP: 228 leader length: 275 |
| 102 | AGI number: AT1G05200.2 uORF ′5 STOP: 162 uORF 3′ STOP: 228 leader length: 275 |
| 103 | AGI number: AT1G05230.4 uORF ′5 STOP: 244 uORF 3′ STOP: 316 leader length: 401 |
| 104 | AGI number: AT1G05230.4 uORF ′5 STOP: 244 uORF 3′ STOP: 316 leader length: 401 |
| 105 | AGI number: AT1G05690.1 uORF ′5 STOP: 188 uORF 3′ STOP: 359 leader length: 350 |
| 106 | AGI number: AT1G05690.1 uORF ′5 STOP: 188 uORF 3′ STOP: 359 leader length: 350 |
| 107 | AGI number: AT1G05710.5 uORF ′5 STOP: 317 uORF 3′ STOP: 383 leader length: 476 |
| 108 | AGI number: AT1G05710.5 uORF ′5 STOP: 317 uORF 3′ STOP: 383 leader length: 476 |
| 109 | AGI number: AT1G05730.1 uORF ′5 STOP: 78 uORF 3′ STOP: 339 leader length: 552 |
| 110 | AGI number: AT1G05730.1 uORF ′5 STOP: 78 uORF 3′ STOP: 339 leader length: 552 |
| 111 | AGI number: AT1G05730.1 uORF ′5 STOP: 260 uORF 3′ STOP: 335 leader length: 552 |
| 112 | AGI number: AT1G05730.1 uORF ′5 STOP: 260 uORF 3′ STOP: 335 leader length: 552 |
| 113 | AGI number: AT1G06149.1 uORF ′5 STOP: 94 uORF 3′ STOP: 172 leader length: 254 |
| 114 | AGI number: AT1G06149.1 uORF ′5 STOP: 94 uORF 3′ STOP: 172 leader length: 254 |
| 115 | AGI number: AT1G06149.1 uORF ′5 STOP: 20 uORF 3′ STOP: 167 leader length: 254 |
| 116 | AGI number: AT1G06149.1 uORF ′5 STOP: 20 uORF 3′ STOP: 167 leader length: 254 |
| 117 | AGI number: AT1G06150.2 uORF ′5 STOP: 228 uORF 3′ STOP: 360 leader length: 524 |
| 118 | AGI number: AT1G06150.2 uORF ′5 STOP: 228 uORF 3′ STOP: 360 leader length: 524 |
| 119 | AGI number: AT1G06150.2 uORF ′5 STOP: 20 uORF 3′ STOP: 167 leader length: 524 |
| 120 | AGI number: AT1G06150.2 uORF ′5 STOP: 20 uORF 3′ STOP: 167 leader length: 524 |
| 121 | AGI number: AT1G06150.2 uORF ′5 STOP: 94 uORF 3′ STOP: 172 leader length: 524 |
| 122 | AGI number: AT1G06150.2 uORF ′5 STOP: 94 uORF 3′ STOP: 172 leader length: 524 |
| 123 | AGI number: AT1G06270.1 uORF ′5 STOP: 231 uORF 3′ STOP: 351 leader length: 334 |
| 124 | AGI number: AT1G06270.1 uORF ′5 STOP: 231 uORF 3′ STOP: 351 leader length: 334 |
| 125 | AGI number: AT1G06270.1 uORF ′5 STOP: 307 uORF 3′ STOP: 391 leader length: 334 |
| 126 | AGI number: AT1G06270.1 uORF ′5 STOP: 307 uORF 3′ STOP: 391 leader length: 334 |
| 127 | AGI number: AT1G06780.2 uORF ′5 STOP: 372 uORF 3′ STOP: 474 leader length: 378 |
| 128 | AGI number: AT1G06780.2 uORF ′5 STOP: 372 uORF 3′ STOP: 474 leader length: 378 |
| 129 | AGI number: AT1G07030.1 uORF ′5 STOP: 113 uORF 3′ STOP: 164 leader length: 285 |
| 130 | AGI number: AT1G07030.1 uORF ′5 STOP: 113 uORF 3′ STOP: 164 leader length: 285 |
| 131 | AGI number: AT1G07590.1 uORF ′5 STOP: 41 uORF 3′ STOP: 209 leader length: 630 |
| 132 | AGI number: AT1G07590.1 uORF ′5 STOP: 41 uORF 3′ STOP: 209 leader length: 630 |
| 133 | AGI number: AT1G07590.1 uORF ′5 STOP: 63 uORF 3′ STOP: 228 leader length: 630 |
| 134 | AGI number: AT1G07590.1 uORF ′5 STOP: 63 uORF 3′ STOP: 228 leader length: 630 |
| 135 | AGI number: AT1G07590.1 uORF ′5 STOP: 343 uORF 3′ STOP: 397 leader length: 630 |
| 136 | AGI number: AT1G07590.1 uORF ′5 STOP: 343 uORF 3′ STOP: 397 leader length: 630 |
| 137 | AGI number: AT1G07700.3 uORF ′5 STOP: 358 uORF 3′ STOP: 553 leader length: 650 |
| 138 | AGI number: AT1G07700.3 uORF ′5 STOP: 358 uORF 3′ STOP: 553 leader length: 650 |
| 139 | AGI number: AT1G07700.3 uORF ′5 STOP: 441 uORF 3′ STOP: 558 leader length: 650 |
| 140 | AGI number: AT1G07700.3 uORF ′5 STOP: 441 uORF 3′ STOP: 558 leader length: 650 |
| 141 | AGI number: AT1G07700.3 uORF ′5 STOP: 582 uORF 3′ STOP: 633 leader length: 650 |
| 142 | AGI number: AT1G07700.3 uORF ′5 STOP: 582 uORF 3′ STOP: 633 leader length: 650 |
| 143 | AGI number: AT1G07780.2 uORF ′5 STOP: 11 uORF 3′ STOP: 71 leader length: 160 |
| 144 | AGI number: AT1G07780.2 uORF ′5 STOP: 11 uORF 3′ STOP: 71 leader length: 160 |
| 145 | AGI number: AT1G07960.2 uORF ′5 STOP: 45 uORF 3′ STOP: 114 leader length: 492 |
| 146 | AGI number: AT1G07960.2 uORF ′5 STOP: 45 uORF 3′ STOP: 114 leader length: 492 |
| 147 | AGI number: AT1G08030.1 uORF ′5 STOP: 55 uORF 3′ STOP: 166 leader length: 267 |
| 148 | AGI number: AT1G08030.1 uORF ′5 STOP: 55 uORF 3′ STOP: 166 leader length: 267 |
| 149 | AGI number: AT1G08600.3 uORF ′5 STOP: 213 uORF 3′ STOP: 270 leader length: 231 |
| 150 | AGI number: AT1G08600.3 uORF ′5 STOP: 213 uORF 3′ STOP: 270 leader length: 231 |
| 151 | AGI number: AT1G08620.1 uORF ′5 STOP: 31 uORF 3′ STOP: 97 leader length: 304 |
| 152 | AGI number: AT1G08620.1 uORF ′5 STOP: 31 uORF 3′ STOP: 97 leader length: 304 |
| 153 | AGI number: AT1G08820.1 uORF ′5 STOP: 20 uORF 3′ STOP: 92 leader length: 297 |
| 154 | AGI number: AT1G08820.1 uORF ′5 STOP: 20 uORF 3′ STOP: 92 leader length: 297 |
| 155 | AGI number: AT1G09180.1 uORF ′5 STOP: 99 uORF 3′ STOP: 168 leader length: 124 |
| 156 | AGI number: AT1G09180.1 uORF ′5 STOP: 99 uORF 3′ STOP: 168 leader length: 124 |
| 157 | AGI number: AT1G09300.2 uORF ′5 STOP: 98 uORF 3′ STOP: 266 leader length: 658 |
| 158 | AGI number: AT1G09300.2 uORF ′5 STOP: 98 uORF 3′ STOP: 266 leader length: 658 |
| 159 | AGI number: AT1G09840.1 uORF ′5 STOP: 158 uORF 3′ STOP: 299 leader length: 343 |
| 160 | AGI number: AT1G09840.1 uORF ′5 STOP: 158 uORF 3′ STOP: 299 leader length: 343 |
| 161 | AGI number: AT1G09840.2 uORF ′5 STOP: 68 uORF 3′ STOP: 191 leader length: 235 |
| 162 | AGI number: AT1G09840.2 uORF ′5 STOP: 68 uORF 3′ STOP: 191 leader length: 235 |
| 163 | AGI number: AT1G09840.6 uORF ′5 STOP: 113 uORF 3′ STOP: 173 leader length: 364 |
| 164 | AGI number: AT1G09840.6 uORF ′5 STOP: 113 uORF 3′ STOP: 173 leader length: 364 |
| 165 | AGI number: AT1G10120.1 uORF ′5 STOP: 25 uORF 3′ STOP: 157 leader length: 840 |
| 166 | AGI number: AT1G10120.1 uORF ′5 STOP: 25 uORF 3′ STOP: 157 leader length: 840 |
| 167 | AGI number: AT1G10522.1 uORF ′5 STOP: 106 uORF 3′ STOP: 217 leader length: 243 |
| 168 | AGI number: AT1G10522.1 uORF ′5 STOP: 106 uORF 3′ STOP: 217 leader length: 243 |
| 169 | AGI number: AT1G10522.1 uORF ′5 STOP: 143 uORF 3′ STOP: 200 leader length: 243 |
| 170 | AGI number: AT1G10522.1 uORF ′5 STOP: 143 uORF 3′ STOP: 200 leader length: 243 |
| 171 | AGI number: AT1G10522.2 uORF ′5 STOP: 74 uORF 3′ STOP: 185 leader length: 259 |
| 172 | AGI number: AT1G10522.2 uORF ′5 STOP: 74 uORF 3′ STOP: 185 leader length: 259 |
| 173 | AGI number: AT1G10650.2 uORF ′5 STOP: 333 uORF 3′ STOP: 528 leader length: 653 |
| 174 | AGI number: AT1G10650.2 uORF ′5 STOP: 333 uORF 3′ STOP: 528 leader length: 653 |
| 175 | AGI number: AT1G10650.2 uORF ′5 STOP: 341 uORF 3′ STOP: 509 leader length: 653 |
| 176 | AGI number: AT1G10650.2 uORF ′5 STOP: 341 uORF 3′ STOP: 509 leader length: 653 |
| 177 | AGI number: AT1G10740.1 uORF ′5 STOP: 27 uORF 3′ STOP: 195 leader length: 294 |
| 178 | AGI number: AT1G10740.1 uORF ′5 STOP: 27 uORF 3′ STOP: 195 leader length: 294 |
| 179 | AGI number: AT1G10740.2 uORF ′5 STOP: 143 uORF 3′ STOP: 203 leader length: 294 |
| 180 | AGI number: AT1G10740.2 uORF ′5 STOP: 143 uORF 3′ STOP: 203 leader length: 294 |
| 181 | AGI number: AT1G10740.2 uORF ′5 STOP: 27 uORF 3′ STOP: 195 leader length: 294 |
| 182 | AGI number: AT1G10740.2 uORF ′5 STOP: 27 uORF 3′ STOP: 195 leader length: 294 |
| 183 | AGI number: AT1G10740.3 uORF ′5 STOP: 105 uORF 3′ STOP: 273 leader length: 372 |
| 184 | AGI number: AT1G10740.3 uORF ′5 STOP: 105 uORF 3′ STOP: 273 leader length: 372 |
| 185 | AGI number: AT1G11170.1 uORF ′5 STOP: 88 uORF 3′ STOP: 148 leader length: 230 |
| 186 | AGI number: AT1G11170.1 uORF ′5 STOP: 88 uORF 3′ STOP: 148 leader length: 230 |
| 187 | AGI number: AT1G11270.1 uORF ′5 STOP: 60 uORF 3′ STOP: 120 leader length: 214 |
| 188 | AGI number: AT1G11270.1 uORF ′5 STOP: 60 uORF 3′ STOP: 120 leader length: 214 |
| 189 | AGI number: AT1G11270.1 uORF ′5 STOP: 65 uORF 3′ STOP: 125 leader length: 214 |
| 190 | AGI number: AT1G11270.1 uORF ′5 STOP: 65 uORF 3′ STOP: 125 leader length: 214 |
| 191 | AGI number: AT1G11450.1 uORF ′5 STOP: 188 uORF 3′ STOP: 305 leader length: 524 |
| 192 | AGI number: AT1G11450.1 uORF ′5 STOP: 188 uORF 3′ STOP: 305 leader length: 524 |
| 193 | AGI number: AT1G11755.1 uORF ′5 STOP: 16 uORF 3′ STOP: 172 leader length: 260 |
| 194 | AGI number: AT1G11755.1 uORF ′5 STOP: 16 uORF 3′ STOP: 172 leader length: 260 |
| 195 | AGI number: AT1G11790.1 uORF ′5 STOP: 86 uORF 3′ STOP: 146 leader length: 228 |
| 196 | AGI number: AT1G11790.1 uORF ′5 STOP: 86 uORF 3′ STOP: 146 leader length: 228 |
| 197 | AGI number: AT1G12260.1 uORF ′5 STOP: 253 uORF 3′ STOP: 361 leader length: 457 |
| 198 | AGI number: AT1G12260.1 uORF ′5 STOP: 253 uORF 3′ STOP: 361 leader length: 457 |
| 199 | AGI number: AT1G12380.1 uORF ′5 STOP: 100 uORF 3′ STOP: 163 leader length: 252 |
| 200 | AGI number: AT1G12380.1 uORF ′5 STOP: 100 uORF 3′ STOP: 163 leader length: 252 |
| 201 | AGI number: AT1G12440.2 uORF ′5 STOP: 79 uORF 3′ STOP: 133 leader length: 641 |
| 202 | AGI number: AT1G12440.2 uORF ′5 STOP: 79 uORF 3′ STOP: 133 leader length: 641 |
| 203 | AGI number: AT1G12440.2 uORF ′5 STOP: 352 uORF 3′ STOP: 424 leader length: 641 |
| 204 | AGI number: AT1G12440.2 uORF ′5 STOP: 352 uORF 3′ STOP: 424 leader length: 641 |
| 205 | AGI number: AT1G12440.2 uORF ′5 STOP: 14 uORF 3′ STOP: 161 leader length: 641 |
| 206 | AGI number: AT1G12440.2 uORF ′5 STOP: 14 uORF 3′ STOP: 161 leader length: 641 |
| 207 | AGI number: AT1G12520.2 uORF ′5 STOP: 58 uORF 3′ STOP: 283 leader length: 368 |
| 208 | AGI number: AT1G12520.2 uORF ′5 STOP: 58 uORF 3′ STOP: 283 leader length: 368 |
| 209 | AGI number: AT1G12520.2 uORF ′5 STOP: 239 uORF 3′ STOP: 296 leader length: 368 |
| 210 | AGI number: AT1G12520.2 uORF ′5 STOP: 239 uORF 3′ STOP: 296 leader length: 368 |
| 211 | AGI number: AT1G12800.1 uORF ′5 STOP: 67 uORF 3′ STOP: 136 leader length: 142 |
| 212 | AGI number: AT1G12800.1 uORF ′5 STOP: 67 uORF 3′ STOP: 136 leader length: 142 |
| 213 | AGI number: AT1G12900.4 uORF ′5 STOP: 162 uORF 3′ STOP: 225 leader length: 304 |
| 214 | AGI number: AT1G12900.4 uORF ′5 STOP: 162 uORF 3′ STOP: 225 leader length: 304 |
| 215 | AGI number: AT1G12990.1 uORF ′5 STOP: 107 uORF 3′ STOP: 203 leader length: 333 |
| 216 | AGI number: AT1G12990.1 uORF ′5 STOP: 107 uORF 3′ STOP: 203 leader length: 333 |
| 217 | AGI number: AT1G13120.1 uORF ′5 STOP: 243 uORF 3′ STOP: 321 leader length: 438 |
| 218 | AGI number: AT1G13120.1 uORF ′5 STOP: 243 uORF 3′ STOP: 321 leader length: 438 |
| 219 | AGI number: AT1G13120.1 uORF ′5 STOP: 142 uORF 3′ STOP: 325 leader length: 438 |
| 220 | AGI number: AT1G13120.1 uORF ′5 STOP: 142 uORF 3′ STOP: 325 leader length: 438 |
| 221 | AGI number: AT1G13195.2 uORF ′5 STOP: 104 uORF 3′ STOP: 299 leader length: 688 |
| 222 | AGI number: AT1G13195.2 uORF ′5 STOP: 104 uORF 3′ STOP: 299 leader length: 688 |
| 223 | AGI number: AT1G13370.1 uORF ′5 STOP: 82 uORF 3′ STOP: 244 leader length: 91 |
| 224 | AGI number: AT1G13370.1 uORF ′5 STOP: 82 uORF 3′ STOP: 244 leader length: 91 |
| 225 | AGI number: AT1G13370.1 uORF ′5 STOP: 66 uORF 3′ STOP: 279 leader length: 91 |
| 226 | AGI number: AT1G13370.1 uORF ′5 STOP: 66 uORF 3′ STOP: 279 leader length: 91 |
| 227 | AGI number: AT1G13460.2 uORF ′5 STOP: 31 uORF 3′ STOP: 148 leader length: 323 |
| 228 | AGI number: AT1G13460.2 uORF ′5 STOP: 31 uORF 3′ STOP: 148 leader length: 323 |
| 229 | AGI number: AT1G13640.1 uORF ′5 STOP: 92 uORF 3′ STOP: 281 leader length: 371 |
| 230 | AGI number: AT1G13640.1 uORF ′5 STOP: 92 uORF 3′ STOP: 281 leader length: 371 |
| 231 | AGI number: AT1G13860.1 uORF ′5 STOP: 389 uORF 3′ STOP: 443 leader length: 562 |
| 232 | AGI number: AT1G13860.1 uORF ′5 STOP: 389 uORF 3′ STOP: 443 leader length: 562 |
| 233 | AGI number: AT1G13860.3 uORF ′5 STOP: 344 uORF 3′ STOP: 398 leader length: 517 |
| 234 | AGI number: AT1G13860.3 uORF ′5 STOP: 344 uORF 3′ STOP: 398 leader length: 517 |
| 235 | AGI number: AT1G13910.1 uORF ′5 STOP: 58 uORF 3′ STOP: 148 leader length: 171 |
| 236 | AGI number: AT1G13910.1 uORF ′5 STOP: 58 uORF 3′ STOP: 148 leader length: 171 |
| 237 | AGI number: AT1G13970.1 uORF ′5 STOP: 155 uORF 3′ STOP: 233 leader length: 316 |
| 238 | AGI number: AT1G13970.1 uORF ′5 STOP: 155 uORF 3′ STOP: 233 leader length: 316 |
| 239 | AGI number: AT1G14020.1 uORF ′5 STOP: 9 uORF 3′ STOP: 222 leader length: 210 |
| 240 | AGI number: AT1G14020.1 uORF ′5 STOP: 9 uORF 3′ STOP: 222 leader length: 210 |
| 241 | AGI number: AT1G14180.2 uORF ′5 STOP: 160 uORF 3′ STOP: 247 leader length: 475 |
| 242 | AGI number: AT1G14180.2 uORF ′5 STOP: 160 uORF 3′ STOP: 247 leader length: 475 |
| 243 | AGI number: AT1G14270.2 uORF ′5 STOP: 196 uORF 3′ STOP: 385 leader length: 572 |
| 244 | AGI number: AT1G14270.2 uORF ′5 STOP: 196 uORF 3′ STOP: 385 leader length: 572 |
| 245 | AGI number: AT1G14560.1 uORF ′5 STOP: 397 uORF 3′ STOP: 619 leader length: 628 |
| 246 | AGI number: AT1G14560.1 uORF ′5 STOP: 397 uORF 3′ STOP: 619 leader length: 628 |
| 247 | AGI number: AT1G14590.1 uORF ′5 STOP: 5 uORF 3′ STOP: 95 leader length: 26 |
| 248 | AGI number: AT1G14590.1 uORF ′5 STOP: 5 uORF 3′ STOP: 95 leader length: 26 |
| 249 | AGI number: AT1G14685.3 uORF ′5 STOP: 298 uORF 3′ STOP: 361 leader length: 498 |
| 250 | AGI number: AT1G14685.3 uORF ′5 STOP: 298 uORF 3′ STOP: 361 leader length: 498 |
| 251 | AGI number: AT1G14820.1 uORF ′5 STOP: 133 uORF 3′ STOP: 235 leader length: 305 |
| 252 | AGI number: AT1G14820.1 uORF ′5 STOP: 133 uORF 3′ STOP: 235 leader length: 305 |
| 253 | AGI number: AT1G15490.1 uORF ′5 STOP: 216 uORF 3′ STOP: 357 leader length: 473 |
| 254 | AGI number: AT1G15490.1 uORF ′5 STOP: 216 uORF 3′ STOP: 357 leader length: 473 |
| 255 | AGI number: AT1G15750.3 uORF ′5 STOP: 160 uORF 3′ STOP: 259 leader length: 437 |
| 256 | AGI number: AT1G15750.3 uORF ′5 STOP: 160 uORF 3′ STOP: 259 leader length: 437 |
| 257 | AGI number: AT1G15920.2 uORF ′5 STOP: 237 uORF 3′ STOP: 345 leader length: 441 |
| 258 | AGI number: AT1G15920.2 uORF ′5 STOP: 237 uORF 3′ STOP: 345 leader length: 441 |
| 259 | AGI number: AT1G15920.2 uORF ′5 STOP: 302 uORF 3′ STOP: 356 leader length: 441 |
| 260 | AGI number: AT1G15920.2 uORF ′5 STOP: 302 uORF 3′ STOP: 356 leader length: 441 |
| 261 | AGI number: AT1G15920.2 uORF ′5 STOP: 151 uORF 3′ STOP: 319 leader length: 441 |
| 262 | AGI number: AT1G15920.2 uORF ′5 STOP: 151 uORF 3′ STOP: 319 leader length: 441 |
| 263 | AGI number: AT1G15970.1 uORF ′5 STOP: 138 uORF 3′ STOP: 231 leader length: 374 |
| 264 | AGI number: AT1G15970.1 uORF ′5 STOP: 138 uORF 3′ STOP: 231 leader length: 374 |
| 265 | AGI number: AT1G15970.1 uORF ′5 STOP: 170 uORF 3′ STOP: 236 leader length: 374 |
| 266 | AGI number: AT1G15970.1 uORF ′5 STOP: 170 uORF 3′ STOP: 236 leader length: 374 |
| 267 | AGI number: AT1G16060.2 uORF ′5 STOP: 197 uORF 3′ STOP: 254 leader length: 460 |
| 268 | AGI number: AT1G16060.2 uORF ′5 STOP: 197 uORF 3′ STOP: 254 leader length: 460 |
| 269 | AGI number: AT1G16250.1 uORF ′5 STOP: 78 uORF 3′ STOP: 135 leader length: 225 |
| 270 | AGI number: AT1G16250.1 uORF ′5 STOP: 78 uORF 3′ STOP: 135 leader length: 225 |
| 271 | AGI number: AT1G17040.1 uORF ′5 STOP: 9 uORF 3′ STOP: 72 leader length: 261 |
| 272 | AGI number: AT1G17040.1 uORF ′5 STOP: 9 uORF 3′ STOP: 72 leader length: 261 |
| 273 | AGI number: AT1G17040.2 uORF ′5 STOP: 260 uORF 3′ STOP: 386 leader length: 261 |
| 274 | AGI number: AT1G17040.2 uORF ′5 STOP: 260 uORF 3′ STOP: 386 leader length: 261 |
| 275 | AGI number: AT1G17680.1 uORF ′5 STOP: 77 uORF 3′ STOP: 242 leader length: 311 |
| 276 | AGI number: AT1G17680.1 uORF ′5 STOP: 77 uORF 3′ STOP: 242 leader length: 311 |
| 277 | AGI number: AT1G17720.1 uORF ′5 STOP: 183 uORF 3′ STOP: 237 leader length: 318 |
| 278 | AGI number: AT1G17720.1 uORF ′5 STOP: 183 uORF 3′ STOP: 237 leader length: 318 |
| 279 | AGI number: AT1G17720.1 uORF ′5 STOP: 163 uORF 3′ STOP: 241 leader length: 318 |
| 280 | AGI number: AT1G17720.1 uORF ′5 STOP: 163 uORF 3′ STOP: 241 leader length: 318 |
| 281 | AGI number: AT1G17720.2 uORF ′5 STOP: 120 uORF 3′ STOP: 198 leader length: 275 |
| 282 | AGI number: AT1G17720.2 uORF ′5 STOP: 120 uORF 3′ STOP: 198 leader length: 275 |
| 283 | AGI number: AT1G17720.2 uORF ′5 STOP: 140 uORF 3′ STOP: 194 leader length: 275 |
| 284 | AGI number: AT1G17720.2 uORF ′5 STOP: 140 uORF 3′ STOP: 194 leader length: 275 |
| 285 | AGI number: AT1G17780.1 uORF ′5 STOP: 382 uORF 3′ STOP: 457 leader length: 602 |
| 286 | AGI number: AT1G17780.1 uORF ′5 STOP: 382 uORF 3′ STOP: 457 leader length: 602 |
| 287 | AGI number: AT1G17890.2 uORF ′5 STOP: 80 uORF 3′ STOP: 137 leader length: 241 |
| 288 | AGI number: AT1G17890.2 uORF ′5 STOP: 80 uORF 3′ STOP: 137 leader length: 241 |
| 289 | AGI number: AT1G17920.1 uORF ′5 STOP: 190 uORF 3′ STOP: 250 leader length: 349 |
| 290 | AGI number: AT1G17920.1 uORF ′5 STOP: 190 uORF 3′ STOP: 250 leader length: 349 |
| 291 | AGI number: AT1G17980.2 uORF ′5 STOP: 364 uORF 3′ STOP: 442 leader length: 706 |
| 292 | AGI number: AT1G17980.2 uORF ′5 STOP: 364 uORF 3′ STOP: 442 leader length: 706 |
| 293 | AGI number: AT1G17980.2 uORF ′5 STOP: 369 uORF 3′ STOP: 468 leader length: 706 |
| 294 | AGI number: AT1G17980.2 uORF ′5 STOP: 369 uORF 3′ STOP: 468 leader length: 706 |
| 295 | AGI number: AT1G17990.1 uORF ′5 STOP: 177 uORF 3′ STOP: 246 leader length: 338 |
| 296 | AGI number: AT1G17990.1 uORF ′5 STOP: 177 uORF 3′ STOP: 246 leader length: 338 |
| 297 | AGI number: AT1G17990.2 uORF ′5 STOP: 76 uORF 3′ STOP: 145 leader length: 237 |
| 298 | AGI number: AT1G17990.2 uORF ′5 STOP: 76 uORF 3′ STOP: 145 leader length: 237 |
| 299 | AGI number: AT1G18160.1 uORF ′5 STOP: 102 uORF 3′ STOP: 183 leader length: 333 |
| 300 | AGI number: AT1G18160.1 uORF ′5 STOP: 102 uORF 3′ STOP: 183 leader length: 333 |
| 301 | AGI number: AT1G18330.2 uORF ′5 STOP: 92 uORF 3′ STOP: 182 leader length: 292 |
| 302 | AGI number: AT1G18330.2 uORF ′5 STOP: 92 uORF 3′ STOP: 182 leader length: 292 |
| 303 | AGI number: AT1G18570.1 uORF ′5 STOP: 126 uORF 3′ STOP: 225 leader length: 354 |
| 304 | AGI number: AT1G18570.1 uORF ′5 STOP: 126 uORF 3′ STOP: 225 leader length: 354 |
| 305 | AGI number: AT1G18690.1 uORF ′5 STOP: 35 uORF 3′ STOP: 296 leader length: 502 |
| 306 | AGI number: AT1G18690.1 uORF ′5 STOP: 35 uORF 3′ STOP: 296 leader length: 502 |
| 307 | AGI number: AT1G18750.1 uORF ′5 STOP: 154 uORF 3′ STOP: 229 leader length: 412 |
| 308 | AGI number: AT1G18750.1 uORF ′5 STOP: 154 uORF 3′ STOP: 229 leader length: 412 |
| 309 | AGI number: AT1G18840.2 uORF ′5 STOP: 100 uORF 3′ STOP: 160 leader length: 395 |
| 310 | AGI number: AT1G18840.2 uORF ′5 STOP: 100 uORF 3′ STOP: 160 leader length: 395 |
| 311 | AGI number: AT1G19220.1 uORF ′5 STOP: 120 uORF 3′ STOP: 198 leader length: 308 |
| 312 | AGI number: AT1G19220.1 uORF ′5 STOP: 120 uORF 3′ STOP: 198 leader length: 308 |
| 313 | AGI number: AT1G19220.1 uORF ′5 STOP: 155 uORF 3′ STOP: 221 leader length: 308 |
| 314 | AGI number: AT1G19220.1 uORF ′5 STOP: 155 uORF 3′ STOP: 221 leader length: 308 |
| 315 | AGI number: AT1G19330.1 uORF ′5 STOP: 110 uORF 3′ STOP: 170 leader length: 239 |
| 316 | AGI number: AT1G19330.1 uORF ′5 STOP: 110 uORF 3′ STOP: 170 leader length: 239 |
| 317 | AGI number: AT1G19370.1 uORF ′5 STOP: 64 uORF 3′ STOP: 154 leader length: 213 |
| 318 | AGI number: AT1G19370.1 uORF ′5 STOP: 64 uORF 3′ STOP: 154 leader length: 213 |
| 319 | AGI number: AT1G19396.3 uORF ′5 STOP: 38 uORF 3′ STOP: 209 leader length: 95 |
| 320 | AGI number: AT1G19396.3 uORF ′5 STOP: 38 uORF 3′ STOP: 209 leader length: 95 |
| 321 | AGI number: AT1G19400.1 uORF ′5 STOP: 301 uORF 3′ STOP: 409 leader length: 571 |
| 322 | AGI number: AT1G19400.1 uORF ′5 STOP: 301 uORF 3′ STOP: 409 leader length: 571 |
| 323 | AGI number: AT1G19450.1 uORF ′5 STOP: 10 uORF 3′ STOP: 61 leader length: 249 |
| 324 | AGI number: AT1G19450.1 uORF ′5 STOP: 10 uORF 3′ STOP: 61 leader length: 249 |
| 325 | AGI number: AT1G19700.2 uORF ′5 STOP: 59 uORF 3′ STOP: 164 leader length: 361 |
| 326 | AGI number: AT1G19700.2 uORF ′5 STOP: 59 uORF 3′ STOP: 164 leader length: 361 |
| 327 | AGI number: AT1G19800.3 uORF ′5 STOP: 45 uORF 3′ STOP: 102 leader length: 266 |
| 328 | AGI number: AT1G19800.3 uORF ′5 STOP: 45 uORF 3′ STOP: 102 leader length: 266 |
| 329 | AGI number: AT1G20550.1 uORF ′5 STOP: 3 uORF 3′ STOP: 354 leader length: 225 |
| 330 | AGI number: AT1G20550.1 uORF ′5 STOP: 3 uORF 3′ STOP: 354 leader length: 225 |
| 331 | AGI number: AT1G20650.1 uORF ′5 STOP: 34 uORF 3′ STOP: 145 leader length: 504 |
| 332 | AGI number: AT1G20650.1 uORF ′5 STOP: 34 uORF 3′ STOP: 145 leader length: 504 |
| 333 | AGI number: AT1G20880.1 uORF ′5 STOP: 85 uORF 3′ STOP: 391 leader length: 604 |
| 334 | AGI number: AT1G20880.1 uORF ′5 STOP: 85 uORF 3′ STOP: 391 leader length: 604 |
| 335 | AGI number: AT1G20880.1 uORF ′5 STOP: 459 uORF 3′ STOP: 513 leader length: 604 |
| 336 | AGI number: AT1G20880.1 uORF ′5 STOP: 459 uORF 3′ STOP: 513 leader length: 604 |
| 337 | AGI number: AT1G21160.1 uORF ′5 STOP: 179 uORF 3′ STOP: 377 leader length: 279 |
| 338 | AGI number: AT1G21160.1 uORF ′5 STOP: 179 uORF 3′ STOP: 377 leader length: 279 |
| 339 | AGI number: AT1G21350.1 uORF ′5 STOP: 92 uORF 3′ STOP: 227 leader length: 420 |
| 340 | AGI number: AT1G21350.1 uORF ′5 STOP: 92 uORF 3′ STOP: 227 leader length: 420 |
| 341 | AGI number: AT1G21560.1 uORF ′5 STOP: 12 uORF 3′ STOP: 105 leader length: 228 |
| 342 | AGI number: AT1G21560.1 uORF ′5 STOP: 12 uORF 3′ STOP: 105 leader length: 228 |
| 343 | AGI number: AT1G21560.3 uORF ′5 STOP: 68 uORF 3′ STOP: 161 leader length: 249 |
| 344 | AGI number: AT1G21560.3 uORF ′5 STOP: 68 uORF 3′ STOP: 161 leader length: 249 |
| 345 | AGI number: AT1G21780.1 uORF ′5 STOP: 134 uORF 3′ STOP: 242 leader length: 349 |
| 346 | AGI number: AT1G21780.1 uORF ′5 STOP: 134 uORF 3′ STOP: 242 leader length: 349 |
| 347 | AGI number: AT1G21780.2 uORF ′5 STOP: 281 uORF 3′ STOP: 389 leader length: 594 |
| 348 | AGI number: AT1G21780.2 uORF ′5 STOP: 281 uORF 3′ STOP: 389 leader length: 594 |
| 349 | AGI number: AT1G21840.1 uORF ′5 STOP: 179 uORF 3′ STOP: 239 leader length: 399 |
| 350 | AGI number: AT1G21840.1 uORF ′5 STOP: 179 uORF 3′ STOP: 239 leader length: 399 |
| 351 | AGI number: AT1G21840.1 uORF ′5 STOP: 150 uORF 3′ STOP: 249 leader length: 399 |
| 352 | AGI number: AT1G21840.1 uORF ′5 STOP: 150 uORF 3′ STOP: 249 leader length: 399 |
| 353 | AGI number: AT1G21930.1 uORF ′5 STOP: 78 uORF 3′ STOP: 366 leader length: 90 |
| 354 | AGI number: AT1G21930.1 uORF ′5 STOP: 78 uORF 3′ STOP: 366 leader length: 90 |
| 355 | AGI number: AT1G22050.1 uORF ′5 STOP: 110 uORF 3′ STOP: 161 leader length: 227 |
| 356 | AGI number: AT1G22050.1 uORF ′5 STOP: 110 uORF 3′ STOP: 161 leader length: 227 |
| 357 | AGI number: AT1G22180.3 uORF ′5 STOP: 575 uORF 3′ STOP: 626 leader length: 882 |
| 358 | AGI number: AT1G22180.3 uORF ′5 STOP: 575 uORF 3′ STOP: 626 leader length: 882 |
| 359 | AGI number: AT1G22190.1 uORF ′5 STOP: 212 uORF 3′ STOP: 275 leader length: 463 |
| 360 | AGI number: AT1G22190.1 uORF ′5 STOP: 212 uORF 3′ STOP: 275 leader length: 463 |
| 361 | AGI number: AT1G22520.2 uORF ′5 STOP: 74 uORF 3′ STOP: 365 leader length: 114 |
| 362 | AGI number: AT1G22520.2 uORF ′5 STOP: 74 uORF 3′ STOP: 365 leader length: 114 |
| 363 | AGI number: AT1G23150.1 uORF ′5 STOP: 216 uORF 3′ STOP: 315 leader length: 399 |
| 364 | AGI number: AT1G23150.1 uORF ′5 STOP: 216 uORF 3′ STOP: 315 leader length: 399 |
| 365 | AGI number: AT1G23150.1 uORF ′5 STOP: 163 uORF 3′ STOP: 322 leader length: 399 |
| 366 | AGI number: AT1G23150.1 uORF ′5 STOP: 163 uORF 3′ STOP: 322 leader length: 399 |
| 367 | AGI number: AT1G23360.3 uORF ′5 STOP: 194 uORF 3′ STOP: 293 leader length: 409 |
| 368 | AGI number: AT1G23360.3 uORF ′5 STOP: 194 uORF 3′ STOP: 293 leader length: 409 |
| 369 | AGI number: AT1G23480.3 uORF ′5 STOP: 179 uORF 3′ STOP: 260 leader length: 536 |
| 370 | AGI number: AT1G23480.3 uORF ′5 STOP: 179 uORF 3′ STOP: 260 leader length: 536 |
| 371 | AGI number: AT1G23730.1 uORF ′5 STOP: 26 uORF 3′ STOP: 122 leader length: 89 |
| 372 | AGI number: AT1G23730.1 uORF ′5 STOP: 26 uORF 3′ STOP: 122 leader length: 89 |
| 373 | AGI number: AT1G24030.2 uORF ′5 STOP: 132 uORF 3′ STOP: 405 leader length: 160 |
| 374 | AGI number: AT1G24030.2 uORF ′5 STOP: 132 uORF 3′ STOP: 405 leader length: 160 |
| 375 | AGI number: AT1G24510.2 uORF ′5 STOP: 257 uORF 3′ STOP: 311 leader length: 587 |
| 376 | AGI number: AT1G24510.2 uORF ′5 STOP: 257 uORF 3′ STOP: 311 leader length: 587 |
| 377 | AGI number: AT1G25054.2 uORF ′5 STOP: 14 uORF 3′ STOP: 125 leader length: 280 |
| 378 | AGI number: AT1G25054.2 uORF ′5 STOP: 14 uORF 3′ STOP: 125 leader length: 280 |
| 379 | AGI number: AT1G25054.2 uORF ′5 STOP: 231 uORF 3′ STOP: 285 leader length: 280 |
| 380 | AGI number: AT1G25054.2 uORF ′5 STOP: 231 uORF 3′ STOP: 285 leader length: 280 |
| 381 | AGI number: AT1G25054.2 uORF ′5 STOP: 91 uORF 3′ STOP: 268 leader length: 280 |
| 382 | AGI number: AT1G25054.2 uORF ′5 STOP: 91 uORF 3′ STOP: 268 leader length: 280 |
| 383 | AGI number: AT1G25280.1 uORF ′5 STOP: 261 uORF 3′ STOP: 336 leader length: 577 |
| 384 | AGI number: AT1G25280.1 uORF ′5 STOP: 261 uORF 3′ STOP: 336 leader length: 577 |
| 385 | AGI number: AT1G25280.1 uORF ′5 STOP: 145 uORF 3′ STOP: 313 leader length: 577 |
| 386 | AGI number: AT1G25280.1 uORF ′5 STOP: 145 uORF 3′ STOP: 313 leader length: 577 |
| 387 | AGI number: AT1G25280.2 uORF ′5 STOP: 119 uORF 3′ STOP: 194 leader length: 1048 |
| 388 | AGI number: AT1G25280.2 uORF ′5 STOP: 119 uORF 3′ STOP: 194 leader length: 1048 |
| 389 | AGI number: AT1G25280.2 uORF ′5 STOP: 3 uORF 3′ STOP: 171 leader length: 1048 |
| 390 | AGI number: AT1G25280.2 uORF ′5 STOP: 3 uORF 3′ STOP: 171 leader length: 1048 |
| 391 | AGI number: AT1G25280.3 uORF ′5 STOP: 80 uORF 3′ STOP: 248 leader length: 1129 |
| 392 | AGI number: AT1G25280.3 uORF ′5 STOP: 80 uORF 3′ STOP: 248 leader length: 1129 |
| 393 | AGI number: AT1G25280.3 uORF ′5 STOP: 196 uORF 3′ STOP: 271 leader length: 1129 |
| 394 | AGI number: AT1G25280.3 uORF ′5 STOP: 196 uORF 3′ STOP: 271 leader length: 1129 |
| 395 | AGI number: AT1G25420.2 uORF ′5 STOP: 214 uORF 3′ STOP: 379 leader length: 470 |
| 396 | AGI number: AT1G25420.2 uORF ′5 STOP: 214 uORF 3′ STOP: 379 leader length: 470 |
| 397 | AGI number: AT1G25420.3 uORF ′5 STOP: 159 uORF 3′ STOP: 243 leader length: 323 |
| 398 | AGI number: AT1G25420.3 uORF ′5 STOP: 159 uORF 3′ STOP: 243 leader length: 323 |
| 399 | AGI number: AT1G25470.1 uORF ′5 STOP: 296 uORF 3′ STOP: 374 leader length: 489 |
| 400 | AGI number: AT1G25470.1 uORF ′5 STOP: 296 uORF 3′ STOP: 374 leader length: 489 |
| 401 | AGI number: AT1G25470.2 uORF ′5 STOP: 290 uORF 3′ STOP: 368 leader length: 483 |
| 402 | AGI number: AT1G25470.2 uORF ′5 STOP: 290 uORF 3′ STOP: 368 leader length: 483 |
| 403 | AGI number: AT1G25500.1 uORF ′5 STOP: 46 uORF 3′ STOP: 112 leader length: 156 |
| 404 | AGI number: AT1G25500.1 uORF ′5 STOP: 46 uORF 3′ STOP: 112 leader length: 156 |
| 405 | AGI number: AT1G25500.3 uORF ′5 STOP: 221 uORF 3′ STOP: 305 leader length: 279 |
| 406 | AGI number: AT1G25500.3 uORF ′5 STOP: 221 uORF 3′ STOP: 305 leader length: 279 |
| 407 | AGI number: AT1G26150.1 uORF ′5 STOP: 332 uORF 3′ STOP: 416 leader length: 415 |
| 408 | AGI number: AT1G26150.1 uORF ′5 STOP: 332 uORF 3′ STOP: 416 leader length: 415 |
| 409 | AGI number: AT1G26150.1 uORF ′5 STOP: 250 uORF 3′ STOP: 442 leader length: 415 |
| 410 | AGI number: AT1G26150.1 uORF ′5 STOP: 250 uORF 3′ STOP: 442 leader length: 415 |
| 411 | AGI number: AT1G26230.2 uORF ′5 STOP: 153 uORF 3′ STOP: 387 leader length: 522 |
| 412 | AGI number: AT1G26230.2 uORF ′5 STOP: 153 uORF 3′ STOP: 387 leader length: 522 |
| 413 | AGI number: AT1G26230.2 uORF ′5 STOP: 178 uORF 3′ STOP: 265 leader length: 522 |
| 414 | AGI number: AT1G26230.2 uORF ′5 STOP: 178 uORF 3′ STOP: 265 leader length: 522 |
| 415 | AGI number: AT1G26230.2 uORF ′5 STOP: 137 uORF 3′ STOP: 269 leader length: 522 |
| 416 | AGI number: AT1G26230.2 uORF ′5 STOP: 137 uORF 3′ STOP: 269 leader length: 522 |
| 417 | AGI number: AT1G26440.2 uORF ′5 STOP: 101 uORF 3′ STOP: 155 leader length: 314 |
| 418 | AGI number: AT1G26440.2 uORF ′5 STOP: 101 uORF 3′ STOP: 155 leader length: 314 |
| 419 | AGI number: AT1G26520.1 uORF ′5 STOP: 27 uORF 3′ STOP: 219 leader length: 306 |
| 420 | AGI number: AT1G26520.1 uORF ′5 STOP: 27 uORF 3′ STOP: 219 leader length: 306 |
| 421 | AGI number: AT1G26530.1 uORF ′5 STOP: 97 uORF 3′ STOP: 262 leader length: 530 |
| 422 | AGI number: AT1G26530.1 uORF ′5 STOP: 97 uORF 3′ STOP: 262 leader length: 530 |
| 423 | AGI number: AT1G26530.1 uORF ′5 STOP: 494 uORF 3′ STOP: 647 leader length: 530 |
| 424 | AGI number: AT1G26530.1 uORF ′5 STOP: 494 uORF 3′ STOP: 647 leader length: 530 |
| 425 | AGI number: AT1G26660.2 uORF ′5 STOP: 123 uORF 3′ STOP: 186 leader length: 124 |
| 426 | AGI number: AT1G26660.2 uORF ′5 STOP: 123 uORF 3′ STOP: 186 leader length: 124 |
| 427 | AGI number: AT1G26850.1 uORF ′5 STOP: 20 uORF 3′ STOP: 134 leader length: 302 |
| 428 | AGI number: AT1G26850.1 uORF ′5 STOP: 20 uORF 3′ STOP: 134 leader length: 302 |
| 429 | AGI number: AT1G26850.1 uORF ′5 STOP: 34 uORF 3′ STOP: 112 leader length: 302 |
| 430 | AGI number: AT1G26850.1 uORF ′5 STOP: 34 uORF 3′ STOP: 112 leader length: 302 |
| 431 | AGI number: AT1G26850.2 uORF ′5 STOP: 223 uORF 3′ STOP: 301 leader length: 427 |
| 432 | AGI number: AT1G26850.2 uORF ′5 STOP: 223 uORF 3′ STOP: 301 leader length: 427 |
| 433 | AGI number: AT1G26850.2 uORF ′5 STOP: 209 uORF 3′ STOP: 323 leader length: 427 |
| 434 | AGI number: AT1G26850.2 uORF ′5 STOP: 209 uORF 3′ STOP: 323 leader length: 427 |
| 435 | AGI number: AT1G26850.3 uORF ′5 STOP: 14 uORF 3′ STOP: 128 leader length: 296 |
| 436 | AGI number: AT1G26850.3 uORF ′5 STOP: 14 uORF 3′ STOP: 128 leader length: 296 |
| 437 | AGI number: AT1G26850.3 uORF ′5 STOP: 28 uORF 3′ STOP: 106 leader length: 296 |
| 438 | AGI number: AT1G26850.3 uORF ′5 STOP: 28 uORF 3′ STOP: 106 leader length: 296 |
| 439 | AGI number: AT1G27460.1 uORF ′5 STOP: 138 uORF 3′ STOP: 222 leader length: 297 |
| 440 | AGI number: AT1G27460.1 uORF ′5 STOP: 138 uORF 3′ STOP: 222 leader length: 297 |
| 441 | AGI number: AT1G27460.1 uORF ′5 STOP: 101 uORF 3′ STOP: 203 leader length: 297 |
| 442 | AGI number: AT1G27460.1 uORF ′5 STOP: 101 uORF 3′ STOP: 203 leader length: 297 |
| 443 | AGI number: AT1G27760.1 uORF ′5 STOP: 163 uORF 3′ STOP: 217 leader length: 311 |
| 444 | AGI number: AT1G27760.1 uORF ′5 STOP: 163 uORF 3′ STOP: 217 leader length: 311 |
| 445 | AGI number: AT1G27760.1 uORF ′5 STOP: 29 uORF 3′ STOP: 242 leader length: 311 |
| 446 | AGI number: AT1G27760.1 uORF ′5 STOP: 29 uORF 3′ STOP: 242 leader length: 311 |
| 447 | AGI number: AT1G27760.2 uORF ′5 STOP: 70 uORF 3′ STOP: 238 leader length: 519 |
| 448 | AGI number: AT1G27760.2 uORF ′5 STOP: 70 uORF 3′ STOP: 238 leader length: 519 |
| 449 | AGI number: AT1G27760.2 uORF ′5 STOP: 12 uORF 3′ STOP: 225 leader length: 519 |
| 450 | AGI number: AT1G27760.2 uORF ′5 STOP: 12 uORF 3′ STOP: 225 leader length: 519 |
| 451 | AGI number: AT1G27760.3 uORF ′5 STOP: 29 uORF 3′ STOP: 242 leader length: 311 |
| 452 | AGI number: AT1G27760.3 uORF ′5 STOP: 29 uORF 3′ STOP: 242 leader length: 311 |
| 453 | AGI number: AT1G27760.3 uORF ′5 STOP: 87 uORF 3′ STOP: 255 leader length: 311 |
| 454 | AGI number: AT1G27760.3 uORF ′5 STOP: 87 uORF 3′ STOP: 255 leader length: 311 |
| 455 | AGI number: AT1G27840.2 uORF ′5 STOP: 70 uORF 3′ STOP: 268 leader length: 107 |
| 456 | AGI number: AT1G27840.2 uORF ′5 STOP: 70 uORF 3′ STOP: 268 leader length: 107 |
| 457 | AGI number: AT1G27840.3 uORF ′5 STOP: 70 uORF 3′ STOP: 268 leader length: 107 |
| 458 | AGI number: AT1G27840.3 uORF ′5 STOP: 70 uORF 3′ STOP: 268 leader length: 107 |
| 459 | AGI number: AT1G27850.1 uORF ′5 STOP: 288 uORF 3′ STOP: 354 leader length: 609 |
| 460 | AGI number: AT1G27850.1 uORF ′5 STOP: 288 uORF 3′ STOP: 354 leader length: 609 |
| 461 | AGI number: AT1G28090.2 uORF ′5 STOP: 72 uORF 3′ STOP: 156 leader length: 282 |
| 462 | AGI number: AT1G28090.2 uORF ′5 STOP: 72 uORF 3′ STOP: 156 leader length: 282 |
| 463 | AGI number: AT1G28090.2 uORF ′5 STOP: 52 uORF 3′ STOP: 226 leader length: 282 |
| 464 | AGI number: AT1G28090.2 uORF ′5 STOP: 52 uORF 3′ STOP: 226 leader length: 282 |
| 465 | AGI number: AT1G28090.2 uORF ′5 STOP: 153 uORF 3′ STOP: 216 leader length: 282 |
| 466 | AGI number: AT1G28090.2 uORF ′5 STOP: 153 uORF 3′ STOP: 216 leader length: 282 |
| 467 | AGI number: AT1G28135.1 uORF ′5 STOP: 2 uORF 3′ STOP: 128 leader length: 71 |
| 468 | AGI number: AT1G28135.1 uORF ′5 STOP: 2 uORF 3′ STOP: 128 leader length: 71 |
| 469 | AGI number: AT1G28260.1 uORF ′5 STOP: 219 uORF 3′ STOP: 294 leader length: 392 |
| 470 | AGI number: AT1G28260.1 uORF ′5 STOP: 219 uORF 3′ STOP: 294 leader length: 392 |
| 471 | AGI number: AT1G28260.1 uORF ′5 STOP: 265 uORF 3′ STOP: 316 leader length: 392 |
| 472 | AGI number: AT1G28260.1 uORF ′5 STOP: 265 uORF 3′ STOP: 316 leader length: 392 |
| 473 | AGI number: AT1G28260.1 uORF ′5 STOP: 242 uORF 3′ STOP: 305 leader length: 392 |
| 474 | AGI number: AT1G28260.1 uORF ′5 STOP: 242 uORF 3′ STOP: 305 leader length: 392 |
| 475 | AGI number: AT1G28560.1 uORF ′5 STOP: 41 uORF 3′ STOP: 119 leader length: 557 |
| 476 | AGI number: AT1G28560.1 uORF ′5 STOP: 41 uORF 3′ STOP: 119 leader length: 557 |
| 477 | AGI number: AT1G28580.2 uORF ′5 STOP: 125 uORF 3′ STOP: 359 leader length: 553 |
| 478 | AGI number: AT1G28580.2 uORF ′5 STOP: 125 uORF 3′ STOP: 359 leader length: 553 |
| 479 | AGI number: AT1G28580.2 uORF ′5 STOP: 219 uORF 3′ STOP: 312 leader length: 553 |
| 480 | AGI number: AT1G28580.2 uORF ′5 STOP: 219 uORF 3′ STOP: 312 leader length: 553 |
| 481 | AGI number: AT1G28710.2 uORF ′5 STOP: 11 uORF 3′ STOP: 89 leader length: 416 |
| 482 | AGI number: AT1G28710.2 uORF ′5 STOP: 11 uORF 3′ STOP: 89 leader length: 416 |
| 483 | AGI number: AT1G28710.3 uORF ′5 STOP: 106 uORF 3′ STOP: 184 leader length: 445 |
| 484 | AGI number: AT1G28710.3 uORF ′5 STOP: 106 uORF 3′ STOP: 184 leader length: 445 |
| 485 | AGI number: AT1G28710.3 uORF ′5 STOP: 441 uORF 3′ STOP: 609 leader length: 445 |
| 486 | AGI number: AT1G28710.3 uORF ′5 STOP: 441 uORF 3′ STOP: 609 leader length: 445 |
| 487 | AGI number: AT1G28710.3 uORF ′5 STOP: 89 uORF 3′ STOP: 215 leader length: 445 |
| 488 | AGI number: AT1G28710.3 uORF ′5 STOP: 89 uORF 3′ STOP: 215 leader length: 445 |
| 489 | AGI number: AT1G29120.1 uORF ′5 STOP: 71 uORF 3′ STOP: 125 leader length: 200 |
| 490 | AGI number: AT1G29120.1 uORF ′5 STOP: 71 uORF 3′ STOP: 125 leader length: 200 |
| 491 | AGI number: AT1G29120.1 uORF ′5 STOP: 22 uORF 3′ STOP: 106 leader length: 200 |
| 492 | AGI number: AT1G29120.1 uORF ′5 STOP: 22 uORF 3′ STOP: 106 leader length: 200 |
| 493 | AGI number: AT1G29120.2 uORF ′5 STOP: 110 uORF 3′ STOP: 164 leader length: 239 |
| 494 | AGI number: AT1G29120.2 uORF ′5 STOP: 110 uORF 3′ STOP: 164 leader length: 239 |
| 495 | AGI number: AT1G29120.2 uORF ′5 STOP: 61 uORF 3′ STOP: 145 leader length: 239 |
| 496 | AGI number: AT1G29120.2 uORF ′5 STOP: 61 uORF 3′ STOP: 145 leader length: 239 |
| 497 | AGI number: AT1G29120.4 uORF ′5 STOP: 21 uORF 3′ STOP: 105 leader length: 199 |
| 498 | AGI number: AT1G29120.4 uORF ′5 STOP: 21 uORF 3′ STOP: 105 leader length: 199 |
| 499 | AGI number: AT1G29120.4 uORF ′5 STOP: 70 uORF 3′ STOP: 124 leader length: 199 |
| 500 | AGI number: AT1G29120.4 uORF ′5 STOP: 70 uORF 3′ STOP: 124 leader length: 199 |
| 501 | AGI number: AT1G29120.5 uORF ′5 STOP: 165 uORF 3′ STOP: 219 leader length: 294 |
| 502 | AGI number: AT1G29120.5 uORF ′5 STOP: 165 uORF 3′ STOP: 219 leader length: 294 |
| 503 | AGI number: AT1G29120.5 uORF ′5 STOP: 116 uORF 3′ STOP: 200 leader length: 294 |
| 504 | AGI number: AT1G29120.5 uORF ′5 STOP: 116 uORF 3′ STOP: 200 leader length: 294 |
| 505 | AGI number: AT1G29390.2 uORF ′5 STOP: 294 uORF 3′ STOP: 345 leader length: 435 |
| 506 | AGI number: AT1G29390.2 uORF ′5 STOP: 294 uORF 3′ STOP: 345 leader length: 435 |
| 507 | AGI number: AT1G29390.2 uORF ′5 STOP: 182 uORF 3′ STOP: 332 leader length: 435 |
| 508 | AGI number: AT1G29390.2 uORF ′5 STOP: 182 uORF 3′ STOP: 332 leader length: 435 |
| 509 | AGI number: AT1G29465.1 uORF ′5 STOP: 189 uORF 3′ STOP: 252 leader length: 293 |
| 510 | AGI number: AT1G29465.1 uORF ′5 STOP: 189 uORF 3′ STOP: 252 leader length: 293 |
| 511 | AGI number: AT1G29465.1 uORF ′5 STOP: 211 uORF 3′ STOP: 274 leader length: 293 |
| 512 | AGI number: AT1G29465.1 uORF ′5 STOP: 211 uORF 3′ STOP: 274 leader length: 293 |
| 513 | AGI number: AT1G29465.1 uORF ′5 STOP: 164 uORF 3′ STOP: 239 leader length: 293 |
| 514 | AGI number: AT1G29465.1 uORF ′5 STOP: 164 uORF 3′ STOP: 239 leader length: 293 |
| 515 | AGI number: AT1G29890.1 uORF ′5 STOP: 315 uORF 3′ STOP: 384 leader length: 420 |
| 516 | AGI number: AT1G29890.1 uORF ′5 STOP: 315 uORF 3′ STOP: 384 leader length: 420 |
| 517 | AGI number: AT1G29890.1 uORF ′5 STOP: 89 uORF 3′ STOP: 398 leader length: 420 |
| 518 | AGI number: AT1G29890.1 uORF ′5 STOP: 89 uORF 3′ STOP: 398 leader length: 420 |
| 519 | AGI number: AT1G29890.1 uORF ′5 STOP: 301 uORF 3′ STOP: 361 leader length: 420 |
| 520 | AGI number: AT1G29890.1 uORF ′5 STOP: 301 uORF 3′ STOP: 361 leader length: 420 |
| 521 | AGI number: AT1G29950.1 uORF ′5 STOP: 179 uORF 3′ STOP: 296 leader length: 993 |
| 522 | AGI number: AT1G29950.1 uORF ′5 STOP: 179 uORF 3′ STOP: 296 leader length: 993 |
| 523 | AGI number: AT1G29950.1 uORF ′5 STOP: 73 uORF 3′ STOP: 277 leader length: 993 |
| 524 | AGI number: AT1G29950.1 uORF ′5 STOP: 73 uORF 3′ STOP: 277 leader length: 993 |
| 525 | AGI number: AT1G29950.1 uORF ′5 STOP: 718 uORF 3′ STOP: 826 leader length: 993 |
| 526 | AGI number: AT1G29950.1 uORF ′5 STOP: 718 uORF 3′ STOP: 826 leader length: 993 |
| 527 | AGI number: AT1G29950.1 uORF ′5 STOP: 552 uORF 3′ STOP: 633 leader length: 993 |
| 528 | AGI number: AT1G29950.1 uORF ′5 STOP: 552 uORF 3′ STOP: 633 leader length: 993 |
| 529 | AGI number: AT1G29950.1 uORF ′5 STOP: 479 uORF 3′ STOP: 638 leader length: 993 |
| 530 | AGI number: AT1G29950.1 uORF ′5 STOP: 479 uORF 3′ STOP: 638 leader length: 993 |
| 531 | AGI number: AT1G29950.2 uORF ′5 STOP: 590 uORF 3′ STOP: 749 leader length: 1104 |
| 532 | AGI number: AT1G29950.2 uORF ′5 STOP: 590 uORF 3′ STOP: 749 leader length: 1104 |
| 533 | AGI number: AT1G29950.2 uORF ′5 STOP: 829 uORF 3′ STOP: 937 leader length: 1104 |
| 534 | AGI number: AT1G29950.2 uORF ′5 STOP: 829 uORF 3′ STOP: 937 leader length: 1104 |
| 535 | AGI number: AT1G29950.2 uORF ′5 STOP: 242 uORF 3′ STOP: 521 leader length: 1104 |
| 536 | AGI number: AT1G29950.2 uORF ′5 STOP: 242 uORF 3′ STOP: 521 leader length: 1104 |
| 537 | AGI number: AT1G29950.2 uORF ′5 STOP: 663 uORF 3′ STOP: 744 leader length: 1104 |
| 538 | AGI number: AT1G29950.2 uORF ′5 STOP: 663 uORF 3′ STOP: 744 leader length: 1104 |
| 539 | AGI number: AT1G29951.1 uORF ′5 STOP: 242 uORF 3′ STOP: 506 leader length: 586 |
| 540 | AGI number: AT1G29951.1 uORF ′5 STOP: 242 uORF 3′ STOP: 506 leader length: 586 |
| 541 | AGI number: AT1G30210.1 uORF ′5 STOP: 139 uORF 3′ STOP: 238 leader length: 411 |
| 542 | AGI number: AT1G30210.1 uORF ′5 STOP: 139 uORF 3′ STOP: 238 leader length: 411 |
| 543 | AGI number: AT1G30270.1 uORF ′5 STOP: 222 uORF 3′ STOP: 363 leader length: 413 |
| 544 | AGI number: AT1G30270.1 uORF ′5 STOP: 222 uORF 3′ STOP: 363 leader length: 413 |
| 545 | AGI number: AT1G30270.1 uORF ′5 STOP: 271 uORF 3′ STOP: 355 leader length: 413 |
| 546 | AGI number: AT1G30270.1 uORF ′5 STOP: 271 uORF 3′ STOP: 355 leader length: 413 |
| 547 | AGI number: AT1G30330.2 uORF ′5 STOP: 554 uORF 3′ STOP: 638 leader length: 716 |
| 548 | AGI number: AT1G30330.2 uORF ′5 STOP: 554 uORF 3′ STOP: 638 leader length: 716 |
| 549 | AGI number: AT1G30650.1 uORF ′5 STOP: 28 uORF 3′ STOP: 163 leader length: 309 |
| 550 | AGI number: AT1G30650.1 uORF ′5 STOP: 28 uORF 3′ STOP: 163 leader length: 309 |
| 551 | AGI number: AT1G30820.1 uORF ′5 STOP: 98 uORF 3′ STOP: 242 leader length: 339 |
| 552 | AGI number: AT1G30820.1 uORF ′5 STOP: 98 uORF 3′ STOP: 242 leader length: 339 |
| 553 | AGI number: AT1G31300.2 uORF ′5 STOP: 154 uORF 3′ STOP: 268 leader length: 416 |
| 554 | AGI number: AT1G31300.2 uORF ′5 STOP: 154 uORF 3′ STOP: 268 leader length: 416 |
| 555 | AGI number: AT1G31460.1 uORF ′5 STOP: 238 uORF 3′ STOP: 397 leader length: 754 |
| 556 | AGI number: AT1G31460.1 uORF ′5 STOP: 238 uORF 3′ STOP: 397 leader length: 754 |
| 557 | AGI number: AT1G31460.1 uORF ′5 STOP: 245 uORF 3′ STOP: 377 leader length: 754 |
| 558 | AGI number: AT1G31460.1 uORF ′5 STOP: 245 uORF 3′ STOP: 377 leader length: 754 |
| 559 | AGI number: AT1G31600.3 uORF ′5 STOP: 331 uORF 3′ STOP: 415 leader length: 467 |
| 560 | AGI number: AT1G31600.3 uORF ′5 STOP: 331 uORF 3′ STOP: 415 leader length: 467 |
| 561 | AGI number: AT1G31880.1 uORF ′5 STOP: 113 uORF 3′ STOP: 260 leader length: 403 |
| 562 | AGI number: AT1G31880.1 uORF ′5 STOP: 113 uORF 3′ STOP: 260 leader length: 403 |
| 563 | AGI number: AT1G31910.1 uORF ′5 STOP: 25 uORF 3′ STOP: 79 leader length: 651 |
| 564 | AGI number: AT1G31910.1 uORF ′5 STOP: 25 uORF 3′ STOP: 79 leader length: 651 |
| 565 | AGI number: AT1G31910.1 uORF ′5 STOP: 45 uORF 3′ STOP: 369 leader length: 651 |
| 566 | AGI number: AT1G31910.1 uORF ′5 STOP: 45 uORF 3′ STOP: 369 leader length: 651 |
| 567 | AGI number: AT1G31910.2 uORF ′5 STOP: 24 uORF 3′ STOP: 78 leader length: 650 |
| 568 | AGI number: AT1G31910.2 uORF ′5 STOP: 24 uORF 3′ STOP: 78 leader length: 650 |
| 569 | AGI number: AT1G31910.2 uORF ′5 STOP: 44 uORF 3′ STOP: 368 leader length: 650 |
| 570 | AGI number: AT1G31910.2 uORF ′5 STOP: 44 uORF 3′ STOP: 368 leader length: 650 |
| 571 | AGI number: AT1G32230.1 uORF ′5 STOP: 241 uORF 3′ STOP: 385 leader length: 488 |
| 572 | AGI number: AT1G32230.1 uORF ′5 STOP: 241 uORF 3′ STOP: 385 leader length: 488 |
| 573 | AGI number: AT1G32230.2 uORF ′5 STOP: 241 uORF 3′ STOP: 385 leader length: 488 |
| 574 | AGI number: AT1G32230.2 uORF ′5 STOP: 241 uORF 3′ STOP: 385 leader length: 488 |
| 575 | AGI number: AT1G32230.3 uORF ′5 STOP: 241 uORF 3′ STOP: 385 leader length: 488 |
| 576 | AGI number: AT1G32230.3 uORF ′5 STOP: 241 uORF 3′ STOP: 385 leader length: 488 |
| 577 | AGI number: AT1G32540.1 uORF ′5 STOP: 122 uORF 3′ STOP: 266 leader length: 749 |
| 578 | AGI number: AT1G32540.1 uORF ′5 STOP: 122 uORF 3′ STOP: 266 leader length: 749 |
| 579 | AGI number: AT1G32540.1 uORF ′5 STOP: 369 uORF 3′ STOP: 435 leader length: 749 |
| 580 | AGI number: AT1G32540.1 uORF ′5 STOP: 369 uORF 3′ STOP: 435 leader length: 749 |
| 581 | AGI number: AT1G32540.1 uORF ′5 STOP: 383 uORF 3′ STOP: 443 leader length: 749 |
| 582 | AGI number: AT1G32540.1 uORF ′5 STOP: 383 uORF 3′ STOP: 443 leader length: 749 |
| 583 | AGI number: AT1G32540.2 uORF ′5 STOP: 100 uORF 3′ STOP: 160 leader length: 256 |
| 584 | AGI number: AT1G32540.2 uORF ′5 STOP: 100 uORF 3′ STOP: 160 leader length: 256 |
| 585 | AGI number: AT1G32540.2 uORF ′5 STOP: 86 uORF 3′ STOP: 152 leader length: 256 |
| 586 | AGI number: AT1G32540.2 uORF ′5 STOP: 86 uORF 3′ STOP: 152 leader length: 256 |
| 587 | AGI number: AT1G32750.1 uORF ′5 STOP: 54 uORF 3′ STOP: 111 leader length: 292 |
| 588 | AGI number: AT1G32750.1 uORF ′5 STOP: 54 uORF 3′ STOP: 111 leader length: 292 |
| 589 | AGI number: AT1G32780.1 uORF ′5 STOP: 155 uORF 3′ STOP: 209 leader length: 331 |
| 590 | AGI number: AT1G32780.1 uORF ′5 STOP: 155 uORF 3′ STOP: 209 leader length: 331 |
| 591 | AGI number: AT1G34190.1 uORF ′5 STOP: 22 uORF 3′ STOP: 151 leader length: 278 |
| 592 | AGI number: AT1G34190.1 uORF ′5 STOP: 22 uORF 3′ STOP: 151 leader length: 278 |
| 593 | AGI number: AT1G35340.2 uORF ′5 STOP: 6 uORF 3′ STOP: 219 leader length: 366 |
| 594 | AGI number: AT1G35340.2 uORF ′5 STOP: 6 uORF 3′ STOP: 219 leader length: 366 |
| 595 | AGI number: AT1G35340.2 uORF ′5 STOP: 65 uORF 3′ STOP: 224 leader length: 366 |
| 596 | AGI number: AT1G35340.2 uORF ′5 STOP: 65 uORF 3′ STOP: 224 leader length: 366 |
| 597 | AGI number: AT1G43690.1 uORF ′5 STOP: 36 uORF 3′ STOP: 165 leader length: 272 |
| 598 | AGI number: AT1G43690.1 uORF ′5 STOP: 36 uORF 3′ STOP: 165 leader length: 272 |
| 599 | AGI number: AT1G44750.1 uORF ′5 STOP: 46 uORF 3′ STOP: 97 leader length: 49 |
| 600 | AGI number: AT1G44750.1 uORF ′5 STOP: 46 uORF 3′ STOP: 97 leader length: 49 |
| 601 | AGI number: AT1G44750.3 uORF ′5 STOP: 174 uORF 3′ STOP: 270 leader length: 534 |
| 602 | AGI number: AT1G44750.3 uORF ′5 STOP: 174 uORF 3′ STOP: 270 leader length: 534 |
| 603 | AGI number: AT1G44750.3 uORF ′5 STOP: 206 uORF 3′ STOP: 260 leader length: 534 |
| 604 | AGI number: AT1G44750.3 uORF ′5 STOP: 206 uORF 3′ STOP: 260 leader length: 534 |
| 605 | AGI number: AT1G44890.1 uORF ′5 STOP: 38 uORF 3′ STOP: 290 leader length: 129 |
| 606 | AGI number: AT1G44890.1 uORF ′5 STOP: 38 uORF 3′ STOP: 290 leader length: 129 |
| 607 | AGI number: AT1G47740.1 uORF ′5 STOP: 205 uORF 3′ STOP: 283 leader length: 301 |
| 608 | AGI number: AT1G47740.1 uORF ′5 STOP: 205 uORF 3′ STOP: 283 leader length: 301 |
| 609 | AGI number: AT1G47740.1 uORF ′5 STOP: 168 uORF 3′ STOP: 309 leader length: 301 |
| 610 | AGI number: AT1G47740.1 uORF ′5 STOP: 168 uORF 3′ STOP: 309 leader length: 301 |
| 611 | AGI number: AT1G48140.2 uORF ′5 STOP: 124 uORF 3′ STOP: 394 leader length: 133 |
| 612 | AGI number: AT1G48140.2 uORF ′5 STOP: 124 uORF 3′ STOP: 394 leader length: 133 |
| 613 | AGI number: AT1G48330.1 uORF ′5 STOP: 29 uORF 3′ STOP: 293 leader length: 60 |
| 614 | AGI number: AT1G48330.1 uORF ′5 STOP: 29 uORF 3′ STOP: 293 leader length: 60 |
| 615 | AGI number: AT1G48635.1 uORF ′5 STOP: 9 uORF 3′ STOP: 96 leader length: 247 |
| 616 | AGI number: AT1G48635.1 uORF ′5 STOP: 9 uORF 3′ STOP: 96 leader length: 247 |
| 617 | AGI number: AT1G49000.1 uORF ′5 STOP: 90 uORF 3′ STOP: 189 leader length: 136 |
| 618 | AGI number: AT1G49000.1 uORF ′5 STOP: 90 uORF 3′ STOP: 189 leader length: 136 |
| 619 | AGI number: AT1G49130.2 uORF ′5 STOP: 25 uORF 3′ STOP: 91 leader length: 211 |
| 620 | AGI number: AT1G49130.2 uORF ′5 STOP: 25 uORF 3′ STOP: 91 leader length: 211 |
| 621 | AGI number: AT1G50360.1 uORF ′5 STOP: 22 uORF 3′ STOP: 211 leader length: 417 |
| 622 | AGI number: AT1G50360.1 uORF ′5 STOP: 22 uORF 3′ STOP: 211 leader length: 417 |
| 623 | AGI number: AT1G50440.2 uORF ′5 STOP: 68 uORF 3′ STOP: 161 leader length: 288 |
| 624 | AGI number: AT1G50440.2 uORF ′5 STOP: 68 uORF 3′ STOP: 161 leader length: 288 |
| 625 | AGI number: AT1G50440.2 uORF ′5 STOP: 43 uORF 3′ STOP: 151 leader length: 288 |
| 626 | AGI number: AT1G50440.2 uORF ′5 STOP: 43 uORF 3′ STOP: 151 leader length: 288 |
| 627 | AGI number: AT1G50460.1 uORF ′5 STOP: 201 uORF 3′ STOP: 291 leader length: 347 |
| 628 | AGI number: AT1G50460.1 uORF ′5 STOP: 201 uORF 3′ STOP: 291 leader length: 347 |
| 629 | AGI number: AT1G51745.2 uORF ′5 STOP: 94 uORF 3′ STOP: 160 leader length: 570 |
| 630 | AGI number: AT1G51745.2 uORF ′5 STOP: 94 uORF 3′ STOP: 160 leader length: 570 |
| 631 | AGI number: AT1G52080.1 uORF ′5 STOP: 209 uORF 3′ STOP: 341 leader length: 394 |
| 632 | AGI number: AT1G52080.1 uORF ′5 STOP: 209 uORF 3′ STOP: 341 leader length: 394 |
| 633 | AGI number: AT1G52100.2 uORF ′5 STOP: 223 uORF 3′ STOP: 277 leader length: 387 |
| 634 | AGI number: AT1G52100.2 uORF ′5 STOP: 223 uORF 3′ STOP: 277 leader length: 387 |
| 635 | AGI number: AT1G52150.1 uORF ′5 STOP: 82 uORF 3′ STOP: 193 leader length: 717 |
| 636 | AGI number: AT1G52150.1 uORF ′5 STOP: 82 uORF 3′ STOP: 193 leader length: 717 |
| 637 | AGI number: AT1G52150.2 uORF ′5 STOP: 82 uORF 3′ STOP: 193 leader length: 717 |
| 638 | AGI number: AT1G52150.2 uORF ′5 STOP: 82 uORF 3′ STOP: 193 leader length: 717 |
| 639 | AGI number: AT1G52370.3 uORF ′5 STOP: 108 uORF 3′ STOP: 201 leader length: 564 |
| 640 | AGI number: AT1G52370.3 uORF ′5 STOP: 108 uORF 3′ STOP: 201 leader length: 564 |
| 641 | AGI number: AT1G52630.2 uORF ′5 STOP: 248 uORF 3′ STOP: 407 leader length: 411 |
| 642 | AGI number: AT1G52630.2 uORF ′5 STOP: 248 uORF 3′ STOP: 407 leader length: 411 |
| 643 | AGI number: AT1G53035.1 uORF ′5 STOP: 25 uORF 3′ STOP: 103 leader length: 233 |
| 644 | AGI number: AT1G53035.1 uORF ′5 STOP: 25 uORF 3′ STOP: 103 leader length: 233 |
| 645 | AGI number: AT1G53035.1 uORF ′5 STOP: 29 uORF 3′ STOP: 116 leader length: 233 |
| 646 | AGI number: AT1G53035.1 uORF ′5 STOP: 29 uORF 3′ STOP: 116 leader length: 233 |
| 647 | AGI number: AT1G53110.1 uORF ′5 STOP: 87 uORF 3′ STOP: 177 leader length: 245 |
| 648 | AGI number: AT1G53110.1 uORF ′5 STOP: 87 uORF 3′ STOP: 177 leader length: 245 |
| 649 | AGI number: AT1G53110.1 uORF ′5 STOP: 2 uORF 3′ STOP: 185 leader length: 245 |
| 650 | AGI number: AT1G53110.1 uORF ′5 STOP: 2 uORF 3′ STOP: 185 leader length: 245 |
| 651 | AGI number: AT1G53200.2 uORF ′5 STOP: 360 uORF 3′ STOP: 447 leader length: 634 |
| 652 | AGI number: AT1G53200.2 uORF ′5 STOP: 360 uORF 3′ STOP: 447 leader length: 634 |
| 653 | AGI number: AT1G53300.1 uORF ′5 STOP: 104 uORF 3′ STOP: 215 leader length: 304 |
| 654 | AGI number: AT1G53300.1 uORF ′5 STOP: 104 uORF 3′ STOP: 215 leader length: 304 |
| 655 | AGI number: AT1G53430.2 uORF ′5 STOP: 87 uORF 3′ STOP: 234 leader length: 720 |
| 656 | AGI number: AT1G53430.2 uORF ′5 STOP: 87 uORF 3′ STOP: 234 leader length: 720 |
| 657 | AGI number: AT1G53490.1 uORF ′5 STOP: 572 uORF 3′ STOP: 656 leader length: 1196 |
| 658 | AGI number: AT1G53490.1 uORF ′5 STOP: 572 uORF 3′ STOP: 656 leader length: 1196 |
| 659 | AGI number: AT1G53490.1 uORF ′5 STOP: 717 uORF 3′ STOP: 819 leader length: 1196 |
| 660 | AGI number: AT1G53490.1 uORF ′5 STOP: 717 uORF 3′ STOP: 819 leader length: 1196 |
| 661 | AGI number: AT1G53490.1 uORF ′5 STOP: 816 uORF 3′ STOP: 1017 leader length: 1196 |
| 662 | AGI number: AT1G53490.1 uORF ′5 STOP: 816 uORF 3′ STOP: 1017 leader length: 1196 |
| 663 | AGI number: AT1G53490.1 uORF ′5 STOP: 653 uORF 3′ STOP: 788 leader length: 1196 |
| 664 | AGI number: AT1G53490.1 uORF ′5 STOP: 653 uORF 3′ STOP: 788 leader length: 1196 |
| 665 | AGI number: AT1G53490.1 uORF ′5 STOP: 685 uORF 3′ STOP: 796 leader length: 1196 |
| 666 | AGI number: AT1G53490.1 uORF ′5 STOP: 685 uORF 3′ STOP: 796 leader length: 1196 |
| 667 | AGI number: AT1G53490.1 uORF ′5 STOP: 529 uORF 3′ STOP: 652 leader length: 1196 |
| 668 | AGI number: AT1G53490.1 uORF ′5 STOP: 529 uORF 3′ STOP: 652 leader length: 1196 |
| 669 | AGI number: AT1G53650.1 uORF ′5 STOP: 161 uORF 3′ STOP: 215 leader length: 323 |
| 670 | AGI number: AT1G53650.1 uORF ′5 STOP: 161 uORF 3′ STOP: 215 leader length: 323 |
| 671 | AGI number: AT1G53780.3 uORF ′5 STOP: 117 uORF 3′ STOP: 177 leader length: 462 |
| 672 | AGI number: AT1G53780.3 uORF ′5 STOP: 117 uORF 3′ STOP: 177 leader length: 462 |
| 673 | AGI number: AT1G54180.1 uORF ′5 STOP: 101 uORF 3′ STOP: 179 leader length: 270 |
| 674 | AGI number: AT1G54180.1 uORF ′5 STOP: 101 uORF 3′ STOP: 179 leader length: 270 |
| 675 | AGI number: AT1G54180.1 uORF ′5 STOP: 60 uORF 3′ STOP: 195 leader length: 270 |
| 676 | AGI number: AT1G54180.1 uORF ′5 STOP: 60 uORF 3′ STOP: 195 leader length: 270 |
| 677 | AGI number: AT1G54360.4 uORF ′5 STOP: 21 uORF 3′ STOP: 174 leader length: 296 |
| 678 | AGI number: AT1G54360.4 uORF ′5 STOP: 21 uORF 3′ STOP: 174 leader length: 296 |
| 679 | AGI number: AT1G54390.5 uORF ′5 STOP: 123 uORF 3′ STOP: 204 leader length: 232 |
| 680 | AGI number: AT1G54390.5 uORF ′5 STOP: 123 uORF 3′ STOP: 204 leader length: 232 |
| 681 | AGI number: AT1G54410.1 uORF ′5 STOP: 42 uORF 3′ STOP: 477 leader length: 166 |
| 682 | AGI number: AT1G54410.1 uORF ′5 STOP: 42 uORF 3′ STOP: 477 leader length: 166 |
| 683 | AGI number: AT1G55120.2 uORF ′5 STOP: 30 uORF 3′ STOP: 174 leader length: 229 |
| 684 | AGI number: AT1G55120.2 uORF ′5 STOP: 30 uORF 3′ STOP: 174 leader length: 229 |
| 685 | AGI number: AT1G55120.2 uORF ′5 STOP: 97 uORF 3′ STOP: 184 leader length: 229 |
| 686 | AGI number: AT1G55120.2 uORF ′5 STOP: 97 uORF 3′ STOP: 184 leader length: 229 |
| 687 | AGI number: AT1G55350.4 uORF ′5 STOP: 291 uORF 3′ STOP: 342 leader length: 327 |
| 688 | AGI number: AT1G55350.4 uORF ′5 STOP: 291 uORF 3′ STOP: 342 leader length: 327 |
| 689 | AGI number: AT1G55350.4 uORF ′5 STOP: 184 uORF 3′ STOP: 313 leader length: 327 |
| 690 | AGI number: AT1G55350.4 uORF ′5 STOP: 184 uORF 3′ STOP: 313 leader length: 327 |
| 691 | AGI number: AT1G55350.5 uORF ′5 STOP: 396 uORF 3′ STOP: 447 leader length: 432 |
| 692 | AGI number: AT1G55350.5 uORF ′5 STOP: 396 uORF 3′ STOP: 447 leader length: 432 |
| 693 | AGI number: AT1G55500.1 uORF ′5 STOP: 73 uORF 3′ STOP: 199 leader length: 551 |
| 694 | AGI number: AT1G55500.1 uORF ′5 STOP: 73 uORF 3′ STOP: 199 leader length: 551 |
| 695 | AGI number: AT1G55580.1 uORF ′5 STOP: 5 uORF 3′ STOP: 122 leader length: 95 |
| 696 | AGI number: AT1G55580.1 uORF ′5 STOP: 5 uORF 3′ STOP: 122 leader length: 95 |
| 697 | AGI number: AT1G55610.1 uORF ′5 STOP: 14 uORF 3′ STOP: 185 leader length: 424 |
| 698 | AGI number: AT1G55610.1 uORF ′5 STOP: 14 uORF 3′ STOP: 185 leader length: 424 |
| 699 | AGI number: AT1G55610.2 uORF ′5 STOP: 43 uORF 3′ STOP: 169 leader length: 532 |
| 700 | AGI number: AT1G55610.2 uORF ′5 STOP: 43 uORF 3′ STOP: 169 leader length: 532 |
| 701 | AGI number: AT1G55610.2 uORF ′5 STOP: 14 uORF 3′ STOP: 188 leader length: 532 |
| 702 | AGI number: AT1G55610.2 uORF ′5 STOP: 14 uORF 3′ STOP: 188 leader length: 532 |
| 703 | AGI number: AT1G55630.1 uORF ′5 STOP: 266 uORF 3′ STOP: 338 leader length: 314 |
| 704 | AGI number: AT1G55630.1 uORF ′5 STOP: 266 uORF 3′ STOP: 338 leader length: 314 |
| 705 | AGI number: AT1G55675.1 uORF ′5 STOP: 258 uORF 3′ STOP: 327 leader length: 444 |
| 706 | AGI number: AT1G55675.1 uORF ′5 STOP: 258 uORF 3′ STOP: 327 leader length: 444 |
| 707 | AGI number: AT1G55675.1 uORF ′5 STOP: 49 uORF 3′ STOP: 241 leader length: 444 |
| 708 | AGI number: AT1G55675.1 uORF ′5 STOP: 49 uORF 3′ STOP: 241 leader length: 444 |
| 709 | AGI number: AT1G55680.1 uORF ′5 STOP: 12 uORF 3′ STOP: 147 leader length: 226 |
| 710 | AGI number: AT1G55680.1 uORF ′5 STOP: 12 uORF 3′ STOP: 147 leader length: 226 |
| 711 | AGI number: AT1G55760.1 uORF ′5 STOP: 198 uORF 3′ STOP: 288 leader length: 547 |
| 712 | AGI number: AT1G55760.1 uORF ′5 STOP: 198 uORF 3′ STOP: 288 leader length: 547 |
| 713 | AGI number: AT1G56045.1 uORF ′5 STOP: 35 uORF 3′ STOP: 182 leader length: 62 |
| 714 | AGI number: AT1G56045.1 uORF ′5 STOP: 35 uORF 3′ STOP: 182 leader length: 62 |
| 715 | AGI number: AT1G57610.2 uORF ′5 STOP: 23 uORF 3′ STOP: 113 leader length: 198 |
| 716 | AGI number: AT1G57610.2 uORF ′5 STOP: 23 uORF 3′ STOP: 113 leader length: 198 |
| 717 | AGI number: AT1G57610.3 uORF ′5 STOP: 23 uORF 3′ STOP: 113 leader length: 198 |
| 718 | AGI number: AT1G57610.3 uORF ′5 STOP: 23 uORF 3′ STOP: 113 leader length: 198 |
| 719 | AGI number: AT1G57700.1 uORF ′5 STOP: 42 uORF 3′ STOP: 150 leader length: 368 |
| 720 | AGI number: AT1G57700.1 uORF ′5 STOP: 42 uORF 3′ STOP: 150 leader length: 368 |
| 721 | AGI number: AT1G58180.3 uORF ′5 STOP: 92 uORF 3′ STOP: 320 leader length: 449 |
| 722 | AGI number: AT1G58180.3 uORF ′5 STOP: 92 uORF 3′ STOP: 320 leader length: 449 |
| 723 | AGI number: AT1G58180.3 uORF ′5 STOP: 162 uORF 3′ STOP: 351 leader length: 449 |
| 724 | AGI number: AT1G58180.3 uORF ′5 STOP: 162 uORF 3′ STOP: 351 leader length: 449 |
| 725 | AGI number: AT1G58180.3 uORF ′5 STOP: 238 uORF 3′ STOP: 334 leader length: 449 |
| 726 | AGI number: AT1G58180.3 uORF ′5 STOP: 238 uORF 3′ STOP: 334 leader length: 449 |
| 727 | AGI number: AT1G58200.1 uORF ′5 STOP: 48 uORF 3′ STOP: 99 leader length: 297 |
| 728 | AGI number: AT1G58200.1 uORF ′5 STOP: 48 uORF 3′ STOP: 99 leader length: 297 |
| 729 | AGI number: AT1G58200.2 uORF ′5 STOP: 29 uORF 3′ STOP: 158 leader length: 343 |
| 730 | AGI number: AT1G58200.2 uORF ′5 STOP: 29 uORF 3′ STOP: 158 leader length: 343 |
| 731 | AGI number: AT1G58200.2 uORF ′5 STOP: 90 uORF 3′ STOP: 141 leader length: 343 |
| 732 | AGI number: AT1G58200.2 uORF ′5 STOP: 90 uORF 3′ STOP: 141 leader length: 343 |
| 733 | AGI number: AT1G58250.1 uORF ′5 STOP: 96 uORF 3′ STOP: 195 leader length: 248 |
| 734 | AGI number: AT1G58250.1 uORF ′5 STOP: 96 uORF 3′ STOP: 195 leader length: 248 |
| 735 | AGI number: AT1G58250.1 uORF ′5 STOP: 145 uORF 3′ STOP: 199 leader length: 248 |
| 736 | AGI number: AT1G58250.1 uORF ′5 STOP: 145 uORF 3′ STOP: 199 leader length: 248 |
| 737 | AGI number: AT1G58350.1 uORF ′5 STOP: 32 uORF 3′ STOP: 329 leader length: 446 |
| 738 | AGI number: AT1G58350.1 uORF ′5 STOP: 32 uORF 3′ STOP: 329 leader length: 446 |
| 739 | AGI number: AT1G58602.2 uORF ′5 STOP: 102 uORF 3′ STOP: 252 leader length: 509 |
| 740 | AGI number: AT1G58602.2 uORF ′5 STOP: 102 uORF 3′ STOP: 252 leader length: 509 |
| 741 | AGI number: AT1G60440.1 uORF ′5 STOP: 20 uORF 3′ STOP: 80 leader length: 173 |
| 742 | AGI number: AT1G60440.1 uORF ′5 STOP: 20 uORF 3′ STOP: 80 leader length: 173 |
| 743 | AGI number: AT1G60850.3 uORF ′5 STOP: 2 uORF 3′ STOP: 56 leader length: 157 |
| 744 | AGI number: AT1G60850.3 uORF ′5 STOP: 2 uORF 3′ STOP: 56 leader length: 157 |
| 745 | AGI number: AT1G60890.1 uORF ′5 STOP: 58 uORF 3′ STOP: 115 leader length: 526 |
| 746 | AGI number: AT1G60890.1 uORF ′5 STOP: 58 uORF 3′ STOP: 115 leader length: 526 |
| 747 | AGI number: AT1G60890.2 uORF ′5 STOP: 58 uORF 3′ STOP: 115 leader length: 366 |
| 748 | AGI number: AT1G60890.2 uORF ′5 STOP: 58 uORF 3′ STOP: 115 leader length: 366 |
| 749 | AGI number: AT1G61150.3 uORF ′5 STOP: 246 uORF 3′ STOP: 318 leader length: 339 |
| 750 | AGI number: AT1G61150.3 uORF ′5 STOP: 246 uORF 3′ STOP: 318 leader length: 339 |
| 751 | AGI number: AT1G61150.7 uORF ′5 STOP: 181 uORF 3′ STOP: 253 leader length: 274 |
| 752 | AGI number: AT1G61150.7 uORF ′5 STOP: 181 uORF 3′ STOP: 253 leader length: 274 |
| 753 | AGI number: AT1G61410.1 uORF ′5 STOP: 231 uORF 3′ STOP: 414 leader length: 397 |
| 754 | AGI number: AT1G61410.1 uORF ′5 STOP: 231 uORF 3′ STOP: 414 leader length: 397 |
| 755 | AGI number: AT1G61520.2 uORF ′5 STOP: 289 uORF 3′ STOP: 379 leader length: 420 |
| 756 | AGI number: AT1G61520.2 uORF ′5 STOP: 289 uORF 3′ STOP: 379 leader length: 420 |
| 757 | AGI number: AT1G61520.2 uORF ′5 STOP: 133 uORF 3′ STOP: 199 leader length: 420 |
| 758 | AGI number: AT1G61520.2 uORF ′5 STOP: 133 uORF 3′ STOP: 199 leader length: 420 |
| 759 | AGI number: AT1G61820.3 uORF ′5 STOP: 206 uORF 3′ STOP: 257 leader length: 329 |
| 760 | AGI number: AT1G61820.3 uORF ′5 STOP: 206 uORF 3′ STOP: 257 leader length: 329 |
| 761 | AGI number: AT1G62305.1 uORF ′5 STOP: 111 uORF 3′ STOP: 267 leader length: 150 |
| 762 | AGI number: AT1G62305.1 uORF ′5 STOP: 111 uORF 3′ STOP: 267 leader length: 150 |
| 763 | AGI number: AT1G62540.1 uORF ′5 STOP: 63 uORF 3′ STOP: 120 leader length: 76 |
| 764 | AGI number: AT1G62540.1 uORF ′5 STOP: 63 uORF 3′ STOP: 120 leader length: 76 |
| 765 | AGI number: AT1G62610.1 uORF ′5 STOP: 225 uORF 3′ STOP: 333 leader length: 537 |
| 766 | AGI number: AT1G62610.1 uORF ′5 STOP: 225 uORF 3′ STOP: 333 leader length: 537 |
| 767 | AGI number: AT1G62610.2 uORF ′5 STOP: 399 uORF 3′ STOP: 453 leader length: 537 |
| 768 | AGI number: AT1G62610.2 uORF ′5 STOP: 399 uORF 3′ STOP: 453 leader length: 537 |
| 769 | AGI number: AT1G62610.3 uORF ′5 STOP: 225 uORF 3′ STOP: 333 leader length: 537 |
| 770 | AGI number: AT1G62610.3 uORF ′5 STOP: 225 uORF 3′ STOP: 333 leader length: 537 |
| 771 | AGI number: AT1G62840.1 uORF ′5 STOP: 38 uORF 3′ STOP: 122 leader length: 65 |
| 772 | AGI number: AT1G62840.1 uORF ′5 STOP: 38 uORF 3′ STOP: 122 leader length: 65 |
| 773 | AGI number: AT1G63010.3 uORF ′5 STOP: 208 uORF 3′ STOP: 274 leader length: 445 |
| 774 | AGI number: AT1G63010.3 uORF ′5 STOP: 208 uORF 3′ STOP: 274 leader length: 445 |
| 775 | AGI number: AT1G63010.4 uORF ′5 STOP: 94 uORF 3′ STOP: 163 leader length: 345 |
| 776 | AGI number: AT1G63010.4 uORF ′5 STOP: 94 uORF 3′ STOP: 163 leader length: 345 |
| 777 | AGI number: AT1G63010.4 uORF ′5 STOP: 54 uORF 3′ STOP: 174 leader length: 345 |
| 778 | AGI number: AT1G63010.4 uORF ′5 STOP: 54 uORF 3′ STOP: 174 leader length: 345 |
| 779 | AGI number: AT1G63010.5 uORF ′5 STOP: 105 uORF 3′ STOP: 225 leader length: 396 |
| 780 | AGI number: AT1G63010.5 uORF ′5 STOP: 105 uORF 3′ STOP: 225 leader length: 396 |
| 781 | AGI number: AT1G63010.5 uORF ′5 STOP: 145 uORF 3′ STOP: 214 leader length: 396 |
| 782 | AGI number: AT1G63010.5 uORF ′5 STOP: 145 uORF 3′ STOP: 214 leader length: 396 |
| 783 | AGI number: AT1G63480.1 uORF ′5 STOP: 282 uORF 3′ STOP: 342 leader length: 452 |
| 784 | AGI number: AT1G63480.1 uORF ′5 STOP: 282 uORF 3′ STOP: 342 leader length: 452 |
| 785 | AGI number: AT1G63670.3 uORF ′5 STOP: 36 uORF 3′ STOP: 111 leader length: 246 |
| 786 | AGI number: AT1G63670.3 uORF ′5 STOP: 36 uORF 3′ STOP: 111 leader length: 246 |
| 787 | AGI number: AT1G64110.1 uORF ′5 STOP: 33 uORF 3′ STOP: 93 leader length: 225 |
| 788 | AGI number: AT1G64110.1 uORF ′5 STOP: 33 uORF 3′ STOP: 93 leader length: 225 |
| 789 | AGI number: AT1G64110.1 uORF ′5 STOP: 22 uORF 3′ STOP: 97 leader length: 225 |
| 790 | AGI number: AT1G64110.1 uORF ′5 STOP: 22 uORF 3′ STOP: 97 leader length: 225 |
| 791 | AGI number: AT1G64110.2 uORF ′5 STOP: 27 uORF 3′ STOP: 102 leader length: 228 |
| 792 | AGI number: AT1G64110.2 uORF ′5 STOP: 27 uORF 3′ STOP: 102 leader length: 228 |
| 793 | AGI number: AT1G64140.1 uORF ′5 STOP: 283 uORF 3′ STOP: 412 leader length: 526 |
| 794 | AGI number: AT1G64140.1 uORF ′5 STOP: 283 uORF 3′ STOP: 412 leader length: 526 |
| 795 | AGI number: AT1G64230.4 uORF ′5 STOP: 106 uORF 3′ STOP: 208 leader length: 130 |
| 796 | AGI number: AT1G64230.4 uORF ′5 STOP: 106 uORF 3′ STOP: 208 leader length: 130 |
| 797 | AGI number: AT1G64230.5 uORF ′5 STOP: 118 uORF 3′ STOP: 241 leader length: 163 |
| 798 | AGI number: AT1G64230.5 uORF ′5 STOP: 118 uORF 3′ STOP: 241 leader length: 163 |
| 799 | AGI number: AT1G64630.1 uORF ′5 STOP: 132 uORF 3′ STOP: 351 leader length: 430 |
| 800 | AGI number: AT1G64630.1 uORF ′5 STOP: 132 uORF 3′ STOP: 351 leader length: 430 |
| 801 | AGI number: AT1G64990.2 uORF ′5 STOP: 41 uORF 3′ STOP: 134 leader length: 220 |
| 802 | AGI number: AT1G64990.2 uORF ′5 STOP: 41 uORF 3′ STOP: 134 leader length: 220 |
| 803 | AGI number: AT1G64990.2 uORF ′5 STOP: 51 uORF 3′ STOP: 141 leader length: 220 |
| 804 | AGI number: AT1G64990.2 uORF ′5 STOP: 51 uORF 3′ STOP: 141 leader length: 220 |
| 805 | AGI number: AT1G65070.2 uORF ′5 STOP: 48 uORF 3′ STOP: 351 leader length: 66 |
| 806 | AGI number: AT1G65070.2 uORF ′5 STOP: 48 uORF 3′ STOP: 351 leader length: 66 |
| 807 | AGI number: AT1G65840.1 uORF ′5 STOP: 65 uORF 3′ STOP: 269 leader length: 393 |
| 808 | AGI number: AT1G65840.1 uORF ′5 STOP: 65 uORF 3′ STOP: 269 leader length: 393 |
| 809 | AGI number: AT1G66660.1 uORF ′5 STOP: 306 uORF 3′ STOP: 369 leader length: 340 |
| 810 | AGI number: AT1G66660.1 uORF ′5 STOP: 306 uORF 3′ STOP: 369 leader length: 340 |
| 811 | AGI number: AT1G67325.2 uORF ′5 STOP: 196 uORF 3′ STOP: 286 leader length: 436 |
| 812 | AGI number: AT1G67325.2 uORF ′5 STOP: 196 uORF 3′ STOP: 286 leader length: 436 |
| 813 | AGI number: AT1G67480.1 uORF ′5 STOP: 211 uORF 3′ STOP: 448 leader length: 536 |
| 814 | AGI number: AT1G67480.1 uORF ′5 STOP: 211 uORF 3′ STOP: 448 leader length: 536 |
| 815 | AGI number: AT1G67480.2 uORF ′5 STOP: 94 uORF 3′ STOP: 331 leader length: 419 |
| 816 | AGI number: AT1G67480.2 uORF ′5 STOP: 94 uORF 3′ STOP: 331 leader length: 419 |
| 817 | AGI number: AT1G67570.1 uORF ′5 STOP: 88 uORF 3′ STOP: 160 leader length: 158 |
| 818 | AGI number: AT1G67570.1 uORF ′5 STOP: 88 uORF 3′ STOP: 160 leader length: 158 |
| 819 | AGI number: AT1G67800.1 uORF ′5 STOP: 249 uORF 3′ STOP: 600 leader length: 304 |
| 820 | AGI number: AT1G67800.1 uORF ′5 STOP: 249 uORF 3′ STOP: 600 leader length: 304 |
| 821 | AGI number: AT1G67900.1 uORF ′5 STOP: 135 uORF 3′ STOP: 192 leader length: 426 |
| 822 | AGI number: AT1G67900.1 uORF ′5 STOP: 135 uORF 3′ STOP: 192 leader length: 426 |
| 823 | AGI number: AT1G68130.2 uORF ′5 STOP: 353 uORF 3′ STOP: 425 leader length: 599 |
| 824 | AGI number: AT1G68130.2 uORF ′5 STOP: 353 uORF 3′ STOP: 425 leader length: 599 |
| 825 | AGI number: AT1G68160.1 uORF ′5 STOP: 126 uORF 3′ STOP: 180 leader length: 269 |
| 826 | AGI number: AT1G68160.1 uORF ′5 STOP: 126 uORF 3′ STOP: 180 leader length: 269 |
| 827 | AGI number: AT1G68410.2 uORF ′5 STOP: 101 uORF 3′ STOP: 209 leader length: 675 |
| 828 | AGI number: AT1G68410.2 uORF ′5 STOP: 101 uORF 3′ STOP: 209 leader length: 675 |
| 829 | AGI number: AT1G68550.1 uORF ′5 STOP: 136 uORF 3′ STOP: 406 leader length: 500 |
| 830 | AGI number: AT1G68550.1 uORF ′5 STOP: 136 uORF 3′ STOP: 406 leader length: 500 |
| 831 | AGI number: AT1G68550.2 uORF ′5 STOP: 146 uORF 3′ STOP: 416 leader length: 510 |
| 832 | AGI number: AT1G68550.2 uORF ′5 STOP: 146 uORF 3′ STOP: 416 leader length: 510 |
| 833 | AGI number: AT1G68920.1 uORF ′5 STOP: 9 uORF 3′ STOP: 90 leader length: 608 |
| 834 | AGI number: AT1G68920.1 uORF ′5 STOP: 9 uORF 3′ STOP: 90 leader length: 608 |
| 835 | AGI number: AT1G68920.3 uORF ′5 STOP: 14 uORF 3′ STOP: 95 leader length: 613 |
| 836 | AGI number: AT1G68920.3 uORF ′5 STOP: 14 uORF 3′ STOP: 95 leader length: 613 |
| 837 | AGI number: AT1G69170.1 uORF ′5 STOP: 72 uORF 3′ STOP: 273 leader length: 389 |
| 838 | AGI number: AT1G69170.1 uORF ′5 STOP: 72 uORF 3′ STOP: 273 leader length: 389 |
| 839 | AGI number: AT1G69690.1 uORF ′5 STOP: 75 uORF 3′ STOP: 153 leader length: 225 |
| 840 | AGI number: AT1G69690.1 uORF ′5 STOP: 75 uORF 3′ STOP: 153 leader length: 225 |
| 841 | AGI number: AT1G69750.1 uORF ′5 STOP: 16 uORF 3′ STOP: 109 leader length: 646 |
| 842 | AGI number: AT1G69750.1 uORF ′5 STOP: 16 uORF 3′ STOP: 109 leader length: 646 |
| 843 | AGI number: AT1G69750.1 uORF ′5 STOP: 178 uORF 3′ STOP: 334 leader length: 646 |
| 844 | AGI number: AT1G69750.1 uORF ′5 STOP: 178 uORF 3′ STOP: 334 leader length: 646 |
| 845 | AGI number: AT1G69750.1 uORF ′5 STOP: 23 uORF 3′ STOP: 98 leader length: 646 |
| 846 | AGI number: AT1G69750.1 uORF ′5 STOP: 23 uORF 3′ STOP: 98 leader length: 646 |
| 847 | AGI number: AT1G69840.3 uORF ′5 STOP: 276 uORF 3′ STOP: 342 leader length: 430 |
| 848 | AGI number: AT1G69840.3 uORF ′5 STOP: 276 uORF 3′ STOP: 342 leader length: 430 |
| 849 | AGI number: AT1G69870.1 uORF ′5 STOP: 21 uORF 3′ STOP: 108 leader length: 181 |
| 850 | AGI number: AT1G69870.1 uORF ′5 STOP: 21 uORF 3′ STOP: 108 leader length: 181 |
| 851 | AGI number: AT1G70160.1 uORF ′5 STOP: 28 uORF 3′ STOP: 136 leader length: 210 |
| 852 | AGI number: AT1G70160.1 uORF ′5 STOP: 28 uORF 3′ STOP: 136 leader length: 210 |
| 853 | AGI number: AT1G70280.1 uORF ′5 STOP: 255 uORF 3′ STOP: 363 leader length: 506 |
| 854 | AGI number: AT1G70280.1 uORF ′5 STOP: 255 uORF 3′ STOP: 363 leader length: 506 |
| 855 | AGI number: AT1G70280.1 uORF ′5 STOP: 328 uORF 3′ STOP: 427 leader length: 506 |
| 856 | AGI number: AT1G70280.1 uORF ′5 STOP: 328 uORF 3′ STOP: 427 leader length: 506 |
| 857 | AGI number: AT1G70340.1 uORF ′5 STOP: 24 uORF 3′ STOP: 108 leader length: 224 |
| 858 | AGI number: AT1G70340.1 uORF ′5 STOP: 24 uORF 3′ STOP: 108 leader length: 224 |
| 859 | AGI number: AT1G70340.1 uORF ′5 STOP: 34 uORF 3′ STOP: 127 leader length: 224 |
| 860 | AGI number: AT1G70340.1 uORF ′5 STOP: 34 uORF 3′ STOP: 127 leader length: 224 |
| 861 | AGI number: AT1G70670.1 uORF ′5 STOP: 35 uORF 3′ STOP: 134 leader length: 248 |
| 862 | AGI number: AT1G70670.1 uORF ′5 STOP: 35 uORF 3′ STOP: 134 leader length: 248 |
| 863 | AGI number: AT1G70780.1 uORF ′5 STOP: 134 uORF 3′ STOP: 338 leader length: 417 |
| 864 | AGI number: AT1G70780.1 uORF ′5 STOP: 134 uORF 3′ STOP: 338 leader length: 417 |
| 865 | AGI number: AT1G70780.1 uORF ′5 STOP: 145 uORF 3′ STOP: 301 leader length: 417 |
| 866 | AGI number: AT1G70780.1 uORF ′5 STOP: 145 uORF 3′ STOP: 301 leader length: 417 |
| 867 | AGI number: AT1G70782.1 uORF ′5 STOP: 134 uORF 3′ STOP: 338 leader length: 147 |
| 868 | AGI number: AT1G70782.1 uORF ′5 STOP: 134 uORF 3′ STOP: 338 leader length: 147 |
| 869 | AGI number: AT1G71020.2 uORF ′5 STOP: 289 uORF 3′ STOP: 343 leader length: 727 |
| 870 | AGI number: AT1G71020.2 uORF ′5 STOP: 289 uORF 3′ STOP: 343 leader length: 727 |
| 871 | AGI number: AT1G71340.1 uORF ′5 STOP: 787 uORF 3′ STOP: 889 leader length: 1017 |
| 872 | AGI number: AT1G71340.1 uORF ′5 STOP: 787 uORF 3′ STOP: 889 leader length: 1017 |
| 873 | AGI number: AT1G71340.1 uORF ′5 STOP: 779 uORF 3′ STOP: 857 leader length: 1017 |
| 874 | AGI number: AT1G71340.1 uORF ′5 STOP: 779 uORF 3′ STOP: 857 leader length: 1017 |
| 875 | AGI number: AT1G71696.1 uORF ′5 STOP: 74 uORF 3′ STOP: 383 leader length: 458 |
| 876 | AGI number: AT1G71696.1 uORF ′5 STOP: 74 uORF 3′ STOP: 383 leader length: 458 |
| 877 | AGI number: AT1G71940.1 uORF ′5 STOP: 88 uORF 3′ STOP: 148 lcader length: 250 |
| 878 | AGI number: AT1G71940.1 uORF ′5 STOP: 88 uORF 3′ STOP: 148 leader length: 250 |
| 879 | AGI number: AT1G71980.1 uORF ′5 STOP: 51 uORF 3′ STOP: 234 leader length: 347 |
| 880 | AGI number: AT1G71980.1 uORF ′5 STOP: 51 uORF 3′ STOP: 234 leader length: 347 |
| 881 | AGI number: AT1G72130.2 uORF ′5 STOP: 187 uORF 3′ STOP: 307 leader length: 444 |
| 882 | AGI number: AT1G72130.2 uORF ′5 STOP: 187 uORF 3′ STOP: 307 leader length: 444 |
| 883 | AGI number: AT1G72130.2 uORF ′5 STOP: 275 uORF 3′ STOP: 341 leader length: 444 |
| 884 | AGI number: AT1G72130.2 uORF ′5 STOP: 275 uORF 3′ STOP: 341 leader length: 444 |
| 885 | AGI number: AT1G72510.1 uORF ′5 STOP: 183 uORF 3′ STOP: 279 leader length: 436 |
| 886 | AGI number: AT1G72510.1 uORF ′5 STOP: 183 uORF 3′ STOP: 279 leader length: 436 |
| 887 | AGI number: AT1G72510.1 uORF ′5 STOP: 242 uORF 3′ STOP: 305 leader length: 436 |
| 888 | AGI number: AT1G72510.1 uORF ′5 STOP: 242 uORF 3′ STOP: 305 leader length: 436 |
| 889 | AGI number: AT1G72510.2 uORF ′5 STOP: 113 uORF 3′ STOP: 170 leader length: 241 |
| 890 | AGI number: AT1G72510.2 uORF ′5 STOP: 113 uORF 3′ STOP: 170 leader length: 241 |
| 891 | AGI number: AT1G72770.1 uORF ′5 STOP: 464 uORF 3′ STOP: 521 leader length: 585 |
| 892 | AGI number: AT1G72770.1 uORF ′5 STOP: 464 uORF 3′ STOP: 521 leader length: 585 |
| 893 | AGI number: AT1G72770.1 uORF ′5 STOP: 394 uORF 3′ STOP: 511 leader length: 585 |
| 894 | AGI number: AT1G72770.1 uORF ′5 STOP: 394 uORF 3′ STOP: 511 leader length: 585 |
| 895 | AGI number: AT1G72770.2 uORF ′5 STOP: 464 uORF 3′ STOP: 521 leader length: 585 |
| 896 | AGI number: AT1G72770.2 uORF ′5 STOP: 464 uORF 3′ STOP: 521 leader length: 585 |
| 897 | AGI number: AT1G72770.2 uORF ′5 STOP: 394 uORF 3′ STOP: 511 leader length: 585 |
| 898 | AGI number: AT1G72770.2 uORF ′5 STOP: 394 uORF 3′ STOP: 511 leader length: 585 |
| 899 | AGI number: AT1G72770.3 uORF ′5 STOP: 464 uORF 3′ STOP: 521 leader length: 585 |
| 900 | AGI number: AT1G72770.3 uORF ′5 STOP: 464 uORF 3′ STOP: 521 leader length: 585 |
| 901 | AGI number: AT1G72770.3 uORF ′5 STOP: 394 uORF 3′ STOP: 511 leader length: 585 |
| 902 | AGI number: AT1G72770.3 uORF ′5 STOP: 394 uORF 3′ STOP: 511 leader length: 585 |
| 903 | AGI number: AT1G72820.1 uORF ′5 STOP: 197 uORF 3′ STOP: 317 leader length: 579 |
| 904 | AGI number: AT1G72820.1 uORF ′5 STOP: 197 uORF 3′ STOP: 317 leader length: 579 |
| 905 | AGI number: AT1G72820.1 uORF ′5 STOP: 437 uORF 3′ STOP: 491 leader length: 579 |
| 906 | AGI number: AT1G72820.1 uORF ′5 STOP: 437 uORF 3′ STOP: 491 leader length: 579 |
| 907 | AGI number: AT1G72820.1 uORF ′5 STOP: 433 uORF 3′ STOP: 484 leader length: 579 |
| 908 | AGI number: AT1G72820.1 uORF ′5 STOP: 433 uORF 3′ STOP: 484 leader length: 579 |
| 909 | AGI number: AT1G72820.1 uORF ′5 STOP: 253 uORF 3′ STOP: 331 leader length: 579 |
| 910 | AGI number: AT1G72820.1 uORF ′5 STOP: 253 uORF 3′ STOP: 331 leader length: 579 |
| 911 | AGI number: AT1G72830.2 uORF ′5 STOP: 111 uORF 3′ STOP: 204 leader length: 425 |
| 912 | AGI number: AT1G72830.2 uORF ′5 STOP: 111 uORF 3′ STOP: 204 leader length: 425 |
| 913 | AGI number: AT1G72990.1 uORF ′5 STOP: 64 uORF 3′ STOP: 151 leader length: 221 |
| 914 | AGI number: AT1G72990.1 uORF ′5 STOP: 64 uORF 3′ STOP: 151 leader length: 221 |
| 915 | AGI number: AT1G73360.1 uORF ′5 STOP: 602 uORF 3′ STOP: 653 leader length: 701 |
| 916 | AGI number: AT1G73360.1 uORF ′5 STOP: 602 uORF 3′ STOP: 653 leader length: 701 |
| 917 | AGI number: AT1G73360.1 uORF ′5 STOP: 544 uORF 3′ STOP: 616 leader length: 701 |
| 918 | AGI number: AT1G73360.1 uORF ′5 STOP: 544 uORF 3′ STOP: 616 leader length: 701 |
| 919 | AGI number: AT1G73370.2 uORF ′5 STOP: 41 uORF 3′ STOP: 104 leader length: 248 |
| 920 | AGI number: AT1G73370.2 uORF ′5 STOP: 41 uORF 3′ STOP: 104 leader length: 248 |
| 921 | AGI number: AT1G73470.2 uORF ′5 STOP: 5 uORF 3′ STOP: 116 leader length: 480 |
| 922 | AGI number: AT1G73470.2 uORF ′5 STOP: 5 uORF 3′ STOP: 116 leader length: 480 |
| 923 | AGI number: AT1G73480.1 uORF ′5 STOP: 71 uORF 3′ STOP: 179 leader length: 327 |
| 924 | AGI number: AT1G73480.1 uORF ′5 STOP: 71 uORF 3′ STOP: 179 leader length: 327 |
| 925 | AGI number: AT1G74160.1 uORF ′5 STOP: 465 uORF 3′ STOP: 588 leader length: 738 |
| 926 | AGI number: AT1G74160.1 uORF ′5 STOP: 465 uORF 3′ STOP: 588 leader length: 738 |
| 927 | AGI number: AT1G74160.1 uORF ′5 STOP: 520 uORF 3′ STOP: 595 leader length: 738 |
| 928 | AGI number: AT1G74160.1 uORF ′5 STOP: 520 uORF 3′ STOP: 595 leader length: 738 |
| 929 | AGI number: AT1G74160.1 uORF ′5 STOP: 124 uORF 3′ STOP: 187 leader length: 738 |
| 930 | AGI number: AT1G74160.1 uORF ′5 STOP: 124 uORF 3′ STOP: 187 leader length: 738 |
| 931 | AGI number: AT1G74370.1 uORF ′5 STOP: 12 uORF 3′ STOP: 183 leader length: 225 |
| 932 | AGI number: AT1G74370.1 uORF ′5 STOP: 12 uORF 3′ STOP: 183 leader length: 225 |
| 933 | AGI number: AT1G74370.1 uORF ′5 STOP: 109 uORF 3′ STOP: 175 leader length: 225 |
| 934 | AGI number: AT1G74370.1 uORF ′5 STOP: 109 uORF 3′ STOP: 175 leader length: 225 |
| 935 | AGI number: AT1G74650.1 uORF ′5 STOP: 63 uORF 3′ STOP: 135 leader length: 302 |
| 936 | AGI number: AT1G74650.1 uORF ′5 STOP: 63 uORF 3′ STOP: 135 leader length: 302 |
| 937 | AGI number: AT1G74650.1 uORF ′5 STOP: 47 uORF 3′ STOP: 131 leader length: 302 |
| 938 | AGI number: AT1G74650.1 uORF ′5 STOP: 47 uORF 3′ STOP: 131 leader length: 302 |
| 939 | AGI number: AT1G74680.1 uORF ′5 STOP: 161 uORF 3′ STOP: 272 leader length: 340 |
| 940 | AGI number: AT1G74680.1 uORF ′5 STOP: 161 uORF 3′ STOP: 272 leader length: 340 |
| 941 | AGI number: AT1G74910.1 uORF ′5 STOP: 32 uORF 3′ STOP: 101 leader length: 312 |
| 942 | AGI number: AT1G74910.1 uORF ′5 STOP: 32 uORF 3′ STOP: 101 leader length: 312 |
| 943 | AGI number: AT1G75190.2 uORF ′5 STOP: 3 uORF 3′ STOP: 126 leader length: 216 |
| 944 | AGI number: AT1G75190.2 uORF ′5 STOP: 3 uORF 3′ STOP: 126 leader length: 216 |
| 945 | AGI number: AT1G75240.1 uORF ′5 STOP: 265 uORF 3′ STOP: 337 leader length: 427 |
| 946 | AGI number: AT1G75240.1 uORF ′5 STOP: 265 uORF 3′ STOP: 337 leader length: 427 |
| 947 | AGI number: AT1G75240.1 uORF ′5 STOP: 73 uORF 3′ STOP: 148 leader length: 427 |
| 948 | AGI number: AT1G75240.1 uORF ′5 STOP: 73 uORF 3′ STOP: 148 leader length: 427 |
| 949 | AGI number: AT1G75240.1 uORF ′5 STOP: 108 uORF 3′ STOP: 162 leader length: 427 |
| 950 | AGI number: AT1G75240.1 uORF ′5 STOP: 108 uORF 3′ STOP: 162 leader length: 427 |
| 951 | AGI number: AT1G75240.1 uORF ′5 STOP: 275 uORF 3′ STOP: 347 leader length: 427 |
| 952 | AGI number: AT1G75240.1 uORF ′5 STOP: 275 uORF 3′ STOP: 347 leader length: 427 |
| 953 | AGI number: AT1G75300.1 uORF ′5 STOP: 36 uORF 3′ STOP: 126 leader length: 57 |
| 954 | AGI number: AT1G75300.1 uORF ′5 STOP: 36 uORF 3′ STOP: 126 leader length: 57 |
| 955 | AGI number: AT1G75340.2 uORF ′5 STOP: 5 uORF 3′ STOP: 113 leader length: 92 |
| 956 | AGI number: AT1G75340.2 uORF ′5 STOP: 5 uORF 3′ STOP: 113 leader length: 92 |
| 957 | AGI number: AT1G75390.1 uORF ′5 STOP: 314 uORF 3′ STOP: 425 leader length: 526 |
| 958 | AGI number: AT1G75390.1 uORF ′5 STOP: 314 uORF 3′ STOP: 425 leader length: 526 |
| 959 | AGI number: AT1G75390.1 uORF ′5 STOP: 301 uORF 3′ STOP: 400 leader length: 526 |
| 960 | AGI number: AT1G75390.1 uORF ′5 STOP: 301 uORF 3′ STOP: 400 leader length: 526 |
| 961 | AGI number: AT1G75390.1 uORF ′5 STOP: 216 uORF 3′ STOP: 384 leader length: 526 |
| 962 | AGI number: AT1G75390.1 uORF ′5 STOP: 216 uORF 3′ STOP: 384 leader length: 526 |
| 963 | AGI number: AT1G75390.2 uORF ′5 STOP: 216 uORF 3′ STOP: 384 leader length: 526 |
| 964 | AGI number: AT1G75390.2 uORF ′5 STOP: 216 uORF 3′ STOP: 384 leader length: 526 |
| 965 | AGI number: AT1G75390.2 uORF ′5 STOP: 314 uORF 3′ STOP: 425 leader length: 526 |
| 966 | AGI number: AT1G75390.2 uORF ′5 STOP: 314 uORF 3′ STOP: 425 leader length: 526 |
| 967 | AGI number: AT1G75390.2 uORF ′5 STOP: 301 uORF 3′ STOP: 400 leader length: 526 |
| 968 | AGI number: AT1G75390.2 uORF ′5 STOP: 301 uORF 3′ STOP: 400 leader length: 526 |
| 969 | AGI number: AT1G75450.2 uORF ′5 STOP: 80 uORF 3′ STOP: 164 leader length: 323 |
| 970 | AGI number: AT1G75450.2 uORF ′5 STOP: 80 uORF 3′ STOP: 164 leader length: 323 |
| 971 | AGI number: AT1G75490.1 uORF ′5 STOP: 345 uORF 3′ STOP: 585 leader length: 543 |
| 972 | AGI number: AT1G75490.1 uORF ′5 STOP: 345 uORF 3′ STOP: 585 leader length: 543 |
| 973 | AGI number: AT1G75600.1 uORF ′5 STOP: 79 uORF 3′ STOP: 241 leader length: 88 |
| 974 | AGI number: AT1G75600.1 uORF ′5 STOP: 79 uORF 3′ STOP: 241 leader length: 88 |
| 975 | AGI number: AT1G75730.1 uORF ′5 STOP: 550 uORF 3′ STOP: 619 leader length: 684 |
| 976 | AGI number: AT1G75730.1 uORF ′5 STOP: 550 uORF 3′ STOP: 619 leader length: 684 |
| 977 | AGI number: AT1G76200.1 uORF ′5 STOP: 14 uORF 3′ STOP: 287 leader length: 101 |
| 978 | AGI number: AT1G76200.1 uORF ′5 STOP: 14 uORF 3′ STOP: 287 leader length: 101 |
| 979 | AGI number: AT1G76710.1 uORF ′5 STOP: 89 uORF 3′ STOP: 395 leader length: 114 |
| 980 | AGI number: AT1G76710.1 uORF ′5 STOP: 89 uORF 3′ STOP: 395 leader length: 114 |
| 981 | AGI number: AT1G76720.1 uORF ′5 STOP: 425 uORF 3′ STOP: 500 leader length: 580 |
| 982 | AGI number: AT1G76720.1 uORF ′5 STOP: 425 uORF 3′ STOP: 500 leader length: 580 |
| 983 | AGI number: AT1G77350.1 uORF ′5 STOP: 84 uORF 3′ STOP: 450 leader length: 90 |
| 984 | AGI number: AT1G77350.1 uORF ′5 STOP: 84 uORF 3′ STOP: 450 leader length: 90 |
| 985 | AGI number: AT1G77350.2 uORF ′5 STOP: 79 uORF 3′ STOP: 475 leader length: 115 |
| 986 | AGI number: AT1G77350.2 uORF ′5 STOP: 79 uORF 3′ STOP: 475 leader length: 115 |
| 987 | AGI number: AT1G77460.1 uORF ′5 STOP: 174 uORF 3′ STOP: 240 leader length: 332 |
| 988 | AGI number: AT1G77460.1 uORF ′5 STOP: 174 uORF 3′ STOP: 240 leader length: 332 |
| 989 | AGI number: AT1G77460.1 uORF ′5 STOP: 155 uORF 3′ STOP: 257 leader length: 332 |
| 990 | AGI number: AT1G77460.1 uORF ′5 STOP: 155 uORF 3′ STOP: 257 leader length: 332 |
| 991 | AGI number: AT1G77460.2 uORF ′5 STOP: 100 uORF 3′ STOP: 166 leader length: 258 |
| 992 | AGI number: AT1G77460.2 uORF ′5 STOP: 100 uORF 3′ STOP: 166 leader length: 258 |
| 993 | AGI number: AT1G77460.2 uORF ′5 STOP: 81 uORF 3′ STOP: 183 leader length: 258 |
| 994 | AGI number: AT1G77460.2 uORF ′5 STOP: 81 uORF 3′ STOP: 183 leader length: 258 |
| 995 | AGI number: AT1G77580.1 uORF ′5 STOP: 703 uORF 3′ STOP: 829 leader length: 898 |
| 996 | AGI number: AT1G77580.1 uORF ′5 STOP: 703 uORF 3′ STOP: 829 leader length: 898 |
| 997 | AGI number: AT1G77580.1 uORF ′5 STOP: 611 uORF 3′ STOP: 800 leader length: 898 |
| 998 | AGI number: AT1G77580.1 uORF ′5 STOP: 611 uORF 3′ STOP: 800 leader length: 898 |
| 999 | AGI number: AT1G77660.1 uORF ′5 STOP: 133 uORF 3′ STOP: 247 leader length: 325 |
| 1000 | AGI number: AT1G77660.1 uORF ′5 STOP: 133 uORF 3′ STOP: 247 leader length: 325 |
| 1001 | AGI number: AT1G77680.1 uORF ′5 STOP: 112 uORF 3′ STOP: 220 leader length: 324 |
| 1002 | AGI number: AT1G77680.1 uORF ′5 STOP: 112 uORF 3′ STOP: 220 leader length: 324 |
| 1003 | AGI number: AT1G78080.1 uORF ′5 STOP: 178 uORF 3′ STOP: 343 leader length: 541 |
| 1004 | AGI number: AT1G78080.1 uORF ′5 STOP: 178 uORF 3′ STOP: 343 leader length: 541 |
| 1005 | AGI number: AT1G78260.1 uORF ′5 STOP: 52 uORF 3′ STOP: 157 leader length: 332 |
| 1006 | AGI number: AT1G78260.1 uORF ′5 STOP: 52 uORF 3′ STOP: 157 leader length: 332 |
| 1007 | AGI number: AT1G78260.2 uORF ′5 STOP: 52 uORF 3′ STOP: 157 leader length: 332 |
| 1008 | AGI number: AT1G78260.2 uORF ′5 STOP: 52 uORF 3′ STOP: 157 leader length: 332 |
| 1009 | AGI number: AT1G78700.1 uORF ′5 STOP: 347 uORF 3′ STOP: 398 leader length: 505 |
| 1010 | AGI number: AT1G78700.1 uORF ′5 STOP: 347 uORF 3′ STOP: 398 leader length: 505 |
| 1011 | AGI number: AT1G78700.1 uORF ′5 STOP: 243 uORF 3′ STOP: 393 leader length: 505 |
| 1012 | AGI number: AT1G78700.1 uORF ′5 STOP: 243 uORF 3′ STOP: 393 leader length: 505 |
| 1013 | AGI number: AT1G78700.1 uORF ′5 STOP: 295 uORF 3′ STOP: 388 leader length: 505 |
| 1014 | AGI number: AT1G78700.1 uORF ′5 STOP: 295 uORF 3′ STOP: 388 leader length: 505 |
| 1015 | AGI number: AT1G78710.2 uORF ′5 STOP: 185 uORF 3′ STOP: 251 leader length: 246 |
| 1016 | AGI number: AT1G78710.2 uORF ′5 STOP: 185 uORF 3′ STOP: 251 leader length: 246 |
| 1017 | AGI number: AT1G78880.1 uORF ′5 STOP: 173 uORF 3′ STOP: 266 leader length: 404 |
| 1018 | AGI number: AT1G78880.1 uORF ′5 STOP: 173 uORF 3′ STOP: 266 leader length: 404 |
| 1019 | AGI number: AT1G79220.1 uORF ′5 STOP: 56 uORF 3′ STOP: 191 leader length: 292 |
| 1020 | AGI number: AT1G79220.1 uORF ′5 STOP: 56 uORF 3′ STOP: 191 leader length: 292 |
| 1021 | AGI number: AT1G79220.1 uORF ′5 STOP: 141 uORF 3′ STOP: 201 leader length: 292 |
| 1022 | AGI number: AT1G79220.1 uORF ′5 STOP: 141 uORF 3′ STOP: 201 leader length: 292 |
| 1023 | AGI number: AT1G79230.3 uORF ′5 STOP: 181 uORF 3′ STOP: 259 leader length: 471 |
| 1024 | AGI number: AT1G79230.3 uORF ′5 STOP: 181 uORF 3′ STOP: 259 leader length: 471 |
| 1025 | AGI number: AT1G79490.1 uORF ′5 STOP: 134 uORF 3′ STOP: 218 leader length: 161 |
| 1026 | AGI number: AT1G79490.1 uORF ′5 STOP: 134 uORF 3′ STOP: 218 leader length: 161 |
| 1027 | AGI number: AT1G79690.1 uORF ′5 STOP: 5 uORF 3′ STOP: 59 leader length: 184 |
| 1028 | AGI number: AT1G79690.1 uORF ′5 STOP: 5 uORF 3′ STOP: 59 leader length: 184 |
| 1029 | AGI number: AT1G79790.2 uORF ′5 STOP: 115 uORF 3′ STOP: 181 leader length: 280 |
| 1030 | AGI number: AT1G79790.2 uORF ′5 STOP: 115 uORF 3′ STOP: 181 leader length: 280 |
| 1031 | AGI number: AT1G79810.1 uORF ′5 STOP: 10 uORF 3′ STOP: 61 leader length: 200 |
| 1032 | AGI number: AT1G79810.1 uORF ′5 STOP: 10 uORF 3′ STOP: 61 leader length: 200 |
| 1033 | AGI number: AT1G79810.2 uORF ′5 STOP: 10 uORF 3′ STOP: 61 leader length: 357 |
| 1034 | AGI number: AT1G79810.2 uORF ′5 STOP: 10 uORF 3′ STOP: 61 leader length: 357 |
| 1035 | AGI number: AT1G80280.1 uORF ′5 STOP: 228 uORF 3′ STOP: 372 leader length: 445 |
| 1036 | AGI number: AT1G80280.1 uORF ′5 STOP: 228 uORF 3′ STOP: 372 leader length: 445 |
| 1037 | AGI number: AT1G80280.1 uORF ′5 STOP: 132 uORF 3′ STOP: 186 leader length: 445 |
| 1038 | AGI number: AT1G80280.1 uORF ′5 STOP: 132 uORF 3′ STOP: 186 leader length: 445 |
| 1039 | AGI number: AT1G80280.1 uORF ′5 STOP: 62 uORF 3′ STOP: 377 leader length: 445 |
| 1040 | AGI number: AT1G80280.1 uORF ′5 STOP: 62 uORF 3′ STOP: 377 leader length: 445 |
| 1041 | AGI number: AT1G80420.4 uORF ′5 STOP: 63 uORF 3′ STOP: 234 leader length: 468 |
| 1042 | AGI number: AT1G80420.4 uORF ′5 STOP: 63 uORF 3′ STOP: 234 leader length: 468 |
| 1043 | AGI number: AT1G80570.3 uORF ′5 STOP: 78 uORF 3′ STOP: 129 leader length: 465 |
| 1044 | AGI number: AT1G80570.3 uORF ′5 STOP: 78 uORF 3′ STOP: 129 leader length: 465 |
| 1045 | AGI number: AT1G80640.1 uORF ′5 STOP: 76 uORF 3′ STOP: 181 leader length: 313 |
| 1046 | AGI number: AT1G80640.1 uORF ′5 STOP: 76 uORF 3′ STOP: 181 leader length: 313 |
| 1047 | AGI number: AT1G80640.1 uORF ′5 STOP: 53 uORF 3′ STOP: 188 leader length: 313 |
| 1048 | AGI number: AT1G80640.1 uORF ′5 STOP: 53 uORF 3′ STOP: 188 leader length: 313 |
| 1049 | AGI number: AT1G80640.1 uORF ′5 STOP: 102 uORF 3′ STOP: 222 leader length: 313 |
| 1050 | AGI number: AT1G80640.1 uORF ′5 STOP: 102 uORF 3′ STOP: 222 leader length: 313 |
| 1051 | AGI number: AT1G80640.2 uORF ′5 STOP: 102 uORF 3′ STOP: 222 leader length: 313 |
| 1052 | AGI number: AT1G80640.2 uORF ′5 STOP: 102 uORF 3′ STOP: 222 leader length: 313 |
| 1053 | AGI number: AT1G80780.1 uORF ′5 STOP: 21 uORF 3′ STOP: 120 leader length: 250 |
| 1054 | AGI number: AT1G80780.1 uORF ′5 STOP: 21 uORF 3′ STOP: 120 leader length: 250 |
| 1055 | AGI number: AT1G80980.1 uORF ′5 STOP: 393 uORF 3′ STOP: 522 leader length: 647 |
| 1056 | AGI number: AT1G80980.1 uORF ′5 STOP: 393 uORF 3′ STOP: 522 leader length: 647 |
| 1057 | AGI number: AT1G80980.1 uORF ′5 STOP: 285 uORF 3′ STOP: 342 leader length: 647 |
| 1058 | AGI number: AT1G80980.1 uORF ′5 STOP: 285 uORF 3′ STOP: 342 leader length: 647 |
| 1059 | AGI number: AT2G01100.3 uORF ′5 STOP: 325 uORF 3′ STOP: 388 leader length: 581 |
| 1060 | AGI number: AT2G01100.3 uORF ′5 STOP: 325 uORF 3′ STOP: 388 leader length: 581 |
| 1061 | AGI number: AT2G01175.1 uORF ′5 STOP: 419 uORF 3′ STOP: 473 leader length: 456 |
| 1062 | AGI number: AT2G01175.1 uORF ′5 STOP: 419 uORF 3′ STOP: 473 leader length: 456 |
| 1063 | AGI number: AT2G01350.2 uORF ′5 STOP: 24 uORF 3′ STOP: 246 leader length: 317 |
| 1064 | AGI number: AT2G01350.2 uORF ′5 STOP: 24 uORF 3′ STOP: 246 leader length: 317 |
| 1065 | AGI number: AT2G01690.1 uORF ′5 STOP: 120 uORF 3′ STOP: 180 leader length: 306 |
| 1066 | AGI number: AT2G01690.1 uORF ′5 STOP: 120 uORF 3′ STOP: 180 leader length: 306 |
| 1067 | AGI number: AT2G01690.1 uORF ′5 STOP: 77 uORF 3′ STOP: 158 leader length: 306 |
| 1068 | AGI number: AT2G01690.1 uORF ′5 STOP: 77 uORF 3′ STOP: 158 leader length: 306 |
| 1069 | AGI number: AT2G01690.2 uORF ′5 STOP: 77 uORF 3′ STOP: 158 leader length: 306 |
| 1070 | AGI number: AT2G01690.2 uORF ′5 STOP: 77 uORF 3′ STOP: 158 leader length: 306 |
| 1071 | AGI number: AT2G01730.1 uORF ′5 STOP: 68 uORF 3′ STOP: 191 leader length: 119 |
| 1072 | AGI number: AT2G01730.1 uORF ′5 STOP: 68 uORF 3′ STOP: 191 leader length: 119 |
| 1073 | AGI number: AT2G01930.2 uORF ′5 STOP: 237 uORF 3′ STOP: 303 leader length: 474 |
| 1074 | AGI number: AT2G01930.2 uORF ′5 STOP: 237 uORF 3′ STOP: 303 leader length: 474 |
| 1075 | AGI number: AT2G01930.2 uORF ′5 STOP: 259 uORF 3′ STOP: 346 leader length: 474 |
| 1076 | AGI number: AT2G01930.2 uORF ′5 STOP: 259 uORF 3′ STOP: 346 leader length: 474 |
| 1077 | AGI number: AT2G02710.1 uORF ′5 STOP: 25 uORF 3′ STOP: 274 leader length: 263 |
| 1078 | AGI number: AT2G02710.1 uORF ′5 STOP: 25 uORF 3′ STOP: 274 leader length: 263 |
| 1079 | AGI number: AT2G02710.2 uORF ′5 STOP: 114 uORF 3′ STOP: 249 leader length: 405 |
| 1080 | AGI number: AT2G02710.2 uORF ′5 STOP: 114 uORF 3′ STOP: 249 leader length: 405 |
| 1081 | AGI number: AT2G02870.1 uORF ′5 STOP: 155 uORF 3′ STOP: 236 leader length: 438 |
| 1082 | AGI number: AT2G02870.1 uORF ′5 STOP: 155 uORF 3′ STOP: 236 leader length: 438 |
| 1083 | AGI number: AT2G02930.1 uORF ′5 STOP: 103 uORF 3′ STOP: 343 leader length: 116 |
| 1084 | AGI number: AT2G02930.1 uORF ′5 STOP: 103 uORF 3′ STOP: 343 leader length: 116 |
| 1085 | AGI number: AT2G02960.5 uORF ′5 STOP: 45 uORF 3′ STOP: 117 leader length: 379 |
| 1086 | AGI number: AT2G02960.5 uORF ′5 STOP: 45 uORF 3′ STOP: 117 leader length: 379 |
| 1087 | AGI number: AT2G02970.1 uORF ′5 STOP: 12 uORF 3′ STOP: 114 leader length: 282 |
| 1088 | AGI number: AT2G02970.1 uORF ′5 STOP: 12 uORF 3′ STOP: 114 leader length: 282 |
| 1089 | AGI number: AT2G03070.1 uORF ′5 STOP: 46 uORF 3′ STOP: 148 leader length: 233 |
| 1090 | AGI number: AT2G03070.1 uORF ′5 STOP: 46 uORF 3′ STOP: 148 leader length: 233 |
| 1091 | AGI number: AT2G03340.1 uORF ′5 STOP: 36 uORF 3′ STOP: 108 leader length: 212 |
| 1092 | AGI number: AT2G03340.1 uORF ′5 STOP: 36 uORF 3′ STOP: 108 leader length: 212 |
| 1093 | AGI number: AT2G03530.1 uORF ′5 STOP: 94 uORF 3′ STOP: 259 leader length: 127 |
| 1094 | AGI number: AT2G03530.1 uORF ′5 STOP: 94 uORF 3′ STOP: 259 leader length: 127 |
| 1095 | AGI number: AT2G03810.2 uORF ′5 STOP: 7 uORF 3′ STOP: 70 leader length: 338 |
| 1096 | AGI number: AT2G03810.2 uORF ′5 STOP: 7 uORF 3′ STOP: 70 leader length: 338 |
| 1097 | AGI number: AT2G03890.1 uORF ′5 STOP: 82 uORF 3′ STOP: 214 leader length: 546 |
| 1098 | AGI number: AT2G03890.1 uORF ′5 STOP: 82 uORF 3′ STOP: 214 leader length: 546 |
| 1099 | AGI number: AT2G03890.1 uORF ′5 STOP: 244 uORF 3′ STOP: 307 leader length: 546 |
| 1100 | AGI number: AT2G03890.1 uORF ′5 STOP: 244 uORF 3′ STOP: 307 leader length: 546 |
| 1101 | AGI number: AT2G03890.2 uORF ′5 STOP: 79 uORF 3′ STOP: 211 leader length: 543 |
| 1102 | AGI number: AT2G03890.2 uORF ′5 STOP: 79 uORF 3′ STOP: 211 leader length: 543 |
| 1103 | AGI number: AT2G04039.2 uORF ′5 STOP: 288 uORF 3′ STOP: 378 leader length: 369 |
| 1104 | AGI number: AT2G04039.2 uORF ′5 STOP: 288 uORF 3′ STOP: 378 leader length: 369 |
| 1105 | AGI number: AT2G04039.2 uORF ′5 STOP: 149 uORF 3′ STOP: 371 leader length: 369 |
| 1106 | AGI number: AT2G04039.2 uORF ′5 STOP: 149 uORF 3′ STOP: 371 leader length: 369 |
| 1107 | AGI number: AT2G04039.2 uORF ′5 STOP: 307 uORF 3′ STOP: 364 leader length: 369 |
| 1108 | AGI number: AT2G04039.2 uORF ′5 STOP: 307 uORF 3′ STOP: 364 leader length: 369 |
| 1109 | AGI number: AT2G04240.2 uORF ′5 STOP: 87 uORF 3′ STOP: 153 leader length: 633 |
| 1110 | AGI number: AT2G04240.2 uORF ′5 STOP: 87 uORF 3′ STOP: 153 leader length: 633 |
| 1111 | AGI number: AT2G04378.1 uORF ′5 STOP: 179 uORF 3′ STOP: 431 leader length: 706 |
| 1112 | AGI number: AT2G04378.1 uORF ′5 STOP: 179 uORF 3′ STOP: 431 leader length: 706 |
| 1113 | AGI number: AT2G04890.1 uORF ′5 STOP: 69 uORF 3′ STOP: 123 leader length: 407 |
| 1114 | AGI number: AT2G04890.1 uORF ′5 STOP: 69 uORF 3′ STOP: 123 leader length: 407 |
| 1115 | AGI number: AT2G04890.1 uORF ′5 STOP: 5 uORF 3′ STOP: 110 leader length: 407 |
| 1116 | AGI number: AT2G04890.1 uORF ′5 STOP: 5 uORF 3′ STOP: 110 leader length: 407 |
| 1117 | AGI number: AT2G04890.1 uORF ′5 STOP: 21 uORF 3′ STOP: 72 leader length: 407 |
| 1118 | AGI number: AT2G04890.1 uORF ′5 STOP: 21 uORF 3′ STOP: 72 leader length: 407 |
| 1119 | AGI number: AT2G04890.1 uORF ′5 STOP: 79 uORF 3′ STOP: 145 leader length: 407 |
| 1120 | AGI number: AT2G04890.1 uORF ′5 STOP: 79 uORF 3′ STOP: 145 leader length: 407 |
| 1121 | AGI number: AT2G05160.1 uORF ′5 STOP: 336 uORF 3′ STOP: 411 leader length: 357 |
| 1122 | AGI number: AT2G05160.1 uORF ′5 STOP: 336 uORF 3′ STOP: 411 leader length: 357 |
| 1123 | AGI number: AT2G05210.1 uORF ′5 STOP: 80 uORF 3′ STOP: 134 leader length: 418 |
| 1124 | AGI number: AT2G05210.1 uORF ′5 STOP: 80 uORF 3′ STOP: 134 leader length: 418 |
| 1125 | AGI number: AT2G05210.1 uORF ′5 STOP: 232 uORF 3′ STOP: 304 leader length: 418 |
| 1126 | AGI number: AT2G05210.1 uORF ′5 STOP: 232 uORF 3′ STOP: 304 leader length: 418 |
| 1127 | AGI number: AT2G05210.1 uORF ′5 STOP: 242 uORF 3′ STOP: 332 leader length: 418 |
| 1128 | AGI number: AT2G05210.1 uORF ′5 STOP: 242 uORF 3′ STOP: 332 leader length: 418 |
| 1129 | AGI number: AT2G05210.2 uORF ′5 STOP: 77 uORF 3′ STOP: 131 leader length: 481 |
| 1130 | AGI number: AT2G05210.2 uORF ′5 STOP: 77 uORF 3′ STOP: 131 leader length: 481 |
| 1131 | AGI number: AT2G05210.2 uORF ′5 STOP: 140 uORF 3′ STOP: 239 leader length: 481 |
| 1132 | AGI number: AT2G05210.2 uORF ′5 STOP: 140 uORF 3′ STOP: 239 leader length: 481 |
| 1133 | AGI number: AT2G05210.3 uORF ′5 STOP: 215 uORF 3′ STOP: 317 leader length: 400 |
| 1134 | AGI number: AT2G05210.3 uORF ′5 STOP: 215 uORF 3′ STOP: 317 leader length: 400 |
| 1135 | AGI number: AT2G05210.3 uORF ′5 STOP: 237 uORF 3′ STOP: 309 leader length: 400 |
| 1136 | AGI number: AT2G05210.3 uORF ′5 STOP: 237 uORF 3′ STOP: 309 leader length: 400 |
| 1137 | AGI number: AT2G05630.2 uORF ′5 STOP: 2 uORF 3′ STOP: 119 leader length: 107 |
| 1138 | AGI number: AT2G05630.2 uORF ′5 STOP: 2 uORF 3′ STOP: 119 leader length: 107 |
| 1139 | AGI number: AT2G05632.1 uORF ′5 STOP: 80 uORF 3′ STOP: 191 leader length: 228 |
| 1140 | AGI number: AT2G05632.1 uORF ′5 STOP: 80 uORF 3′ STOP: 191 leader length: 228 |
| 1141 | AGI number: AT2G05632.1 uORF ′5 STOP: 27 uORF 3′ STOP: 177 leader length: 228 |
| 1142 | AGI number: AT2G05632.1 uORF ′5 STOP: 27 uORF 3′ STOP: 177 leader length: 228 |
| 1143 | AGI number: AT2G07727.1 uORF ′5 STOP: 98 uORF 3′ STOP: 152 leader length: 285 |
| 1144 | AGI number: AT2G07727.1 uORF ′5 STOP: 98 uORF 3′ STOP: 152 leader length: 285 |
| 1145 | AGI number: AT2G12200.1 uORF ′5 STOP: 35 uORF 3′ STOP: 110 leader length: 38 |
| 1146 | AGI number: AT2G12200.1 uORF ′5 STOP: 35 uORF 3′ STOP: 110 leader length: 38 |
| 1147 | AGI number: AT2G12280.1 uORF ′5 STOP: 2 uORF 3′ STOP: 215 leader length: 42 |
| 1148 | AGI number: AT2G12280.1 uORF ′5 STOP: 2 uORF 3′ STOP: 215 leader length: 42 |
| 1149 | AGI number: AT2G13660.1 uORF ′5 STOP: 109 uORF 3′ STOP: 319 leader length: 127 |
| 1150 | AGI number: AT2G13660.1 uORF ′5 STOP: 109 uORF 3′ STOP: 319 leader length: 127 |
| 1151 | AGI number: AT2G15530.3 uORF ′5 STOP: 151 uORF 3′ STOP: 265 leader length: 546 |
| 1152 | AGI number: AT2G15530.3 uORF ′5 STOP: 151 uORF 3′ STOP: 265 leader length: 546 |
| 1153 | AGI number: AT2G15530.3 uORF ′5 STOP: 243 uORF 3′ STOP: 300 leader length: 546 |
| 1154 | AGI number: AT2G15530.3 uORF ′5 STOP: 243 uORF 3′ STOP: 300 leader length: 546 |
| 1155 | AGI number: AT2G16400.1 uORF ′5 STOP: 313 uORF 3′ STOP: 364 leader length: 547 |
| 1156 | AGI number: AT2G16400.1 uORF ′5 STOP: 313 uORF 3′ STOP: 364 leader length: 547 |
| 1157 | AGI number: AT2G16400.1 uORF ′5 STOP: 287 uORF 3′ STOP: 359 leader length: 547 |
| 1158 | AGI number: AT2G16400.1 uORF ′5 STOP: 287 uORF 3′ STOP: 359 leader length: 547 |
| 1159 | AGI number: AT2G16430.1 uORF ′5 STOP: 283 uORF 3′ STOP: 430 leader length: 502 |
| 1160 | AGI number: AT2G16430.1 uORF ′5 STOP: 283 uORF 3′ STOP: 430 leader length: 502 |
| 1161 | AGI number: AT2G16430.1 uORF ′5 STOP: 332 uORF 3′ STOP: 446 leader length: 502 |
| 1162 | AGI number: AT2G16430.1 uORF ′5 STOP: 332 uORF 3′ STOP: 446 leader length: 502 |
| 1163 | AGI number: AT2G16586.1 uORF ′5 STOP: 475 uORF 3′ STOP: 613 leader length: 712 |
| 1164 | AGI number: AT2G16586.1 uORF ′5 STOP: 475 uORF 3′ STOP: 613 leader length: 712 |
| 1165 | AGI number: AT2G16586.1 uORF ′5 STOP: 419 uORF 3′ STOP: 632 leader length: 712 |
| 1166 | AGI number: AT2G16586.1 uORF ′5 STOP: 419 uORF 3′ STOP: 632 leader length: 712 |
| 1167 | AGI number: AT2G16900.3 uORF ′5 STOP: 342 uORF 3′ STOP: 393 leader length: 682 |
| 1168 | AGI number: AT2G16900.3 uORF ′5 STOP: 342 uORF 3′ STOP: 393 leader length: 682 |
| 1169 | AGI number: AT2G16900.3 uORF ′5 STOP: 362 uORF 3′ STOP: 428 leader length: 682 |
| 1170 | AGI number: AT2G16900.3 uORF ′5 STOP: 362 uORF 3′ STOP: 428 leader length: 682 |
| 1171 | AGI number: AT2G17370.1 uORF ′5 STOP: 13 uORF 3′ STOP: 130 leader length: 232 |
| 1172 | AGI number: AT2G17370.1 uORF ′5 STOP: 13 uORF 3′ STOP: 130 leader length: 232 |
| 1173 | AGI number: AT2G17440.1 uORF ′5 STOP: 10 uORF 3′ STOP: 121 leader length: 191 |
| 1174 | AGI number: AT2G17440.1 uORF ′5 STOP: 10 uORF 3′ STOP: 121 leader length: 191 |
| 1175 | AGI number: AT2G17480.1 uORF ′5 STOP: 186 uORF 3′ STOP: 243 leader length: 322 |
| 1176 | AGI number: AT2G17480.1 uORF ′5 STOP: 186 uORF 3′ STOP: 243 leader length: 322 |
| 1177 | AGI number: AT2G17540.3 uORF ′5 STOP: 62 uORF 3′ STOP: 122 leader length: 225 |
| 1178 | AGI number: AT2G17540.3 uORF ′5 STOP: 62 uORF 3′ STOP: 122 leader length: 225 |
| 1179 | AGI number: AT2G17550.1 uORF ′5 STOP: 175 uORF 3′ STOP: 226 leader length: 620 |
| 1180 | AGI number: AT2G17550.1 uORF ′5 STOP: 175 uORF 3′ STOP: 226 leader length: 620 |
| 1181 | AGI number: AT2G17550.2 uORF ′5 STOP: 583 uORF 3′ STOP: 787 leader length: 945 |
| 1182 | AGI number: AT2G17550.2 uORF ′5 STOP: 583 uORF 3′ STOP: 787 leader length: 945 |
| 1183 | AGI number: AT2G17550.2 uORF ′5 STOP: 465 uORF 3′ STOP: 795 leader length: 945 |
| 1184 | AGI number: AT2G17550.2 uORF ′5 STOP: 465 uORF 3′ STOP: 795 leader length: 945 |
| 1185 | AGI number: AT2G18150.1 uORF ′5 STOP: 63 uORF 3′ STOP: 225 leader length: 67 |
| 1186 | AGI number: AT2G18150.1 uORF ′5 STOP: 63 uORF 3′ STOP: 225 leader length: 67 |
| 1187 | AGI number: AT2G18160.1 uORF ′5 STOP: 263 uORF 3′ STOP: 344 leader length: 439 |
| 1188 | AGI number: AT2G18160.1 uORF ′5 STOP: 263 uORF 3′ STOP: 344 leader length: 439 |
| 1189 | AGI number: AT2G18160.1 uORF ′5 STOP: 168 uORF 3′ STOP: 327 leader length: 439 |
| 1190 | AGI number: AT2G18160.1 uORF ′5 STOP: 168 uORF 3′ STOP: 327 leader length: 439 |
| 1191 | AGI number: AT2G18876.2 uORF ′5 STOP: 64 uORF 3′ STOP: 148 leader length: 592 |
| 1192 | AGI number: AT2G18876.2 uORF ′5 STOP: 64 uORF 3′ STOP: 148 leader length: 592 |
| 1193 | AGI number: AT2G18970.1 uORF ′5 STOP: 211 uORF 3′ STOP: 355 leader length: 257 |
| 1194 | AGI number: AT2G18970.1 uORF ′5 STOP: 211 uORF 3′ STOP: 355 leader length: 257 |
| 1195 | AGI number: AT2G19170.1 uORF ′5 STOP: 84 uORF 3′ STOP: 147 leader length: 309 |
| 1196 | AGI number: AT2G19170.1 uORF ′5 STOP: 84 uORF 3′ STOP: 147 leader length: 309 |
| 1197 | AGI number: AT2G19170.1 uORF ′5 STOP: 94 uORF 3′ STOP: 169 leader length: 309 |
| 1198 | AGI number: AT2G19170.1 uORF ′5 STOP: 94 uORF 3′ STOP: 169 leader length: 309 |
| 1199 | AGI number: AT2G19390.1 uORF ′5 STOP: 6 uORF 3′ STOP: 132 leader length: 442 |
| 1200 | AGI number: AT2G19390.1 uORF ′5 STOP: 6 uORF 3′ STOP: 132 leader length: 442 |
| 1201 | AGI number: AT2G19390.1 uORF ′5 STOP: 80 uORF 3′ STOP: 158 leader length: 442 |
| 1202 | AGI number: AT2G19390.1 uORF ′5 STOP: 80 uORF 3′ STOP: 158 leader length: 442 |
| 1203 | AGI number: AT2G19470.1 uORF ′5 STOP: 4 uORF 3′ STOP: 88 leader length: 258 |
| 1204 | AGI number: AT2G19470.1 uORF ′5 STOP: 4 uORF 3′ STOP: 88 leader length: 258 |
| 1205 | AGI number: AT2G19490.1 uORF ′5 STOP: 51 uORF 3′ STOP: 117 leader length: 205 |
| 1206 | AGI number: AT2G19490.1 uORF ′5 STOP: 51 uORF 3′ STOP: 117 leader length: 205 |
| 1207 | AGI number: AT2G19880.1 uORF ′5 STOP: 49 uORF 3′ STOP: 103 leader length: 220 |
| 1208 | AGI number: AT2G19880.1 uORF ′5 STOP: 49 uORF 3′ STOP: 103 leader length: 220 |
| 1209 | AGI number: AT2G20010.1 uORF ′5 STOP: 20 uORF 3′ STOP: 134 leader length: 543 |
| 1210 | AGI number: AT2G20010.1 uORF ′5 STOP: 20 uORF 3′ STOP: 134 leader length: 543 |
| 1211 | AGI number: AT2G20010.2 uORF ′5 STOP: 102 uORF 3′ STOP: 216 leader length: 372 |
| 1212 | AGI number: AT2G20010.2 uORF ′5 STOP: 102 uORF 3′ STOP: 216 leader length: 372 |
| 1213 | AGI number: AT2G20080.1 uORF ′5 STOP: 101 uORF 3′ STOP: 152 leader length: 291 |
| 1214 | AGI number: AT2G20080.1 uORF ′5 STOP: 101 uORF 3′ STOP: 152 leader length: 291 |
| 1215 | AGI number: AT2G20080.2 uORF ′5 STOP: 101 uORF 3′ STOP: 152 leader length: 291 |
| 1216 | AGI number: AT2G20080.2 uORF ′5 STOP: 101 uORF 3′ STOP: 152 leader length: 291 |
| 1217 | AGI number: AT2G20440.1 uORF ′5 STOP: 32 uORF 3′ STOP: 110 leader length: 140 |
| 1218 | AGI number: AT2G20440.1 uORF ′5 STOP: 32 uORF 3′ STOP: 110 leader length: 140 |
| 1219 | AGI number: AT2G20680.1 uORF ′5 STOP: 194 uORF 3′ STOP: 302 leader length: 557 |
| 1220 | AGI number: AT2G20680.1 uORF ′5 STOP: 194 uORF 3′ STOP: 302 leader length: 557 |
| 1221 | AGI number: AT2G20780.1 uORF ′5 STOP: 33 uORF 3′ STOP: 132 leader length: 243 |
| 1222 | AGI number: AT2G20780.1 uORF ′5 STOP: 33 uORF 3′ STOP: 132 leader length: 243 |
| 1223 | AGI number: AT2G20815.2 uORF ′5 STOP: 314 uORF 3′ STOP: 365 leader length: 733 |
| 1224 | AGI number: AT2G20815.2 uORF ′5 STOP: 314 uORF 3′ STOP: 365 leader length: 733 |
| 1225 | AGI number: AT2G20830.1 uORF ′5 STOP: 544 uORF 3′ STOP: 727 leader length: 832 |
| 1226 | AGI number: AT2G20830.1 uORF ′5 STOP: 544 uORF 3′ STOP: 727 leader length: 832 |
| 1227 | AGI number: AT2G20830.1 uORF ′5 STOP: 125 uORF 3′ STOP: 584 leader length: 832 |
| 1228 | AGI number: AT2G20830.1 uORF ′5 STOP: 125 uORF 3′ STOP: 584 leader length: 832 |
| 1229 | AGI number: AT2G20990.2 uORF ′5 STOP: 4 uORF 3′ STOP: 133 leader length: 275 |
| 1230 | AGI number: AT2G20990.2 uORF ′5 STOP: 4 uORF 3′ STOP: 133 leader length: 275 |
| 1231 | AGI number: AT2G20990.3 uORF ′5 STOP: 4 uORF 3′ STOP: 133 leader length: 275 |
| 1232 | AGI number: AT2G20990.3 uORF ′5 STOP: 4 uORF 3′ STOP: 133 leader length: 275 |
| 1233 | AGI number: AT2G21300.1 uORF ′5 STOP: 91 uORF 3′ STOP: 163 leader length: 242 |
| 1234 | AGI number: AT2G21300.1 uORF ′5 STOP: 91 uORF 3′ STOP: 163 leader length: 242 |
| 1235 | AGI number: AT2G21520.1 uORF ′5 STOP: 177 uORF 3′ STOP: 306 leader length: 437 |
| 1236 | AGI number: AT2G21520.1 uORF ′5 STOP: 177 uORF 3′ STOP: 306 leader length: 437 |
| 1237 | AGI number: AT2G21940.2 uORF ′5 STOP: 160 uORF 3′ STOP: 211 leader length: 277 |
| 1238 | AGI number: AT2G21940.2 uORF ′5 STOP: 160 uORF 3′ STOP: 211 leader length: 277 |
| 1239 | AGI number: AT2G22670.2 uORF ′5 STOP: 61 uORF 3′ STOP: 154 leader length: 228 |
| 1240 | AGI number: AT2G22670.2 uORF ′5 STOP: 61 uORF 3′ STOP: 154 leader length: 228 |
| 1241 | AGI number: AT2G22670.3 uORF ′5 STOP: 9 uORF 3′ STOP: 102 leader length: 179 |
| 1242 | AGI number: AT2G22670.3 uORF ′5 STOP: 9 uORF 3′ STOP: 102 leader length: 179 |
| 1243 | AGI number: AT2G22670.4 uORF ′5 STOP: 121 uORF 3′ STOP: 214 leader length: 327 |
| 1244 | AGI number: AT2G22670.4 uORF ′5 STOP: 121 uORF 3′ STOP: 214 leader length: 327 |
| 1245 | AGI number: AT2G22670.4 uORF ′5 STOP: 32 uORF 3′ STOP: 284 leader length: 327 |
| 1246 | AGI number: AT2G22670.4 uORF ′5 STOP: 32 uORF 3′ STOP: 284 leader length: 327 |
| 1247 | AGI number: AT2G22670.4 uORF ′5 STOP: 60 uORF 3′ STOP: 159 leader length: 327 |
| 1248 | AGI number: AT2G22670.4 uORF ′5 STOP: 60 uORF 3′ STOP: 159 leader length: 327 |
| 1249 | AGI number: AT2G23680.2 uORF ′5 STOP: 74 uORF 3′ STOP: 224 leader length: 297 |
| 1250 | AGI number: AT2G23680.2 uORF ′5 STOP: 74 uORF 3′ STOP: 224 leader length: 297 |
| 1251 | AGI number: AT2G23700.1 uORF ′5 STOP: 138 uORF 3′ STOP: 264 leader length: 383 |
| 1252 | AGI number: AT2G23700.1 uORF ′5 STOP: 138 uORF 3′ STOP: 264 leader length: 383 |
| 1253 | AGI number: AT2G23985.1 uORF ′5 STOP: 22 uORF 3′ STOP: 94 leader length: 298 |
| 1254 | AGI number: AT2G23985.1 uORF ′5 STOP: 22 uORF 3′ STOP: 94 leader length: 298 |
| 1255 | AGI number: AT2G23985.2 uORF ′5 STOP: 113 uORF 3′ STOP: 206 leader length: 310 |
| 1256 | AGI number: AT2G23985.2 uORF ′5 STOP: 113 uORF 3′ STOP: 206 leader length: 310 |
| 1257 | AGI number: AT2G23985.2 uORF ′5 STOP: 22 uORF 3′ STOP: 94 leader length: 310 |
| 1258 | AGI number: AT2G23985.2 uORF ′5 STOP: 22 uORF 3′ STOP: 94 leader length: 310 |
| 1259 | AGI number: AT2G24280.1 uORF ′5 STOP: 213 uORF 3′ STOP: 327 leader length: 1338 |
| 1260 | AGI number: AT2G24280.1 uORF ′5 STOP: 213 uORF 3′ STOP: 327 leader length: 1338 |
| 1261 | AGI number: AT2G24280.1 uORF ′5 STOP: 307 uORF 3′ STOP: 424 leader length: 1338 |
| 1262 | AGI number: AT2G24280.1 uORF ′5 STOP: 307 uORF 3′ STOP: 424 leader length: 1338 |
| 1263 | AGI number: AT2G24280.1 uORF ′5 STOP: 269 uORF 3′ STOP: 323 leader length: 1338 |
| 1264 | AGI number: AT2G24280.1 uORF ′5 STOP: 269 uORF 3′ STOP: 323 leader length: 1338 |
| 1265 | AGI number: AT2G24390.3 uORF ′5 STOP: 26 uORF 3′ STOP: 95 leader length: 226 |
| 1266 | AGI number: AT2G24390.3 uORF ′5 STOP: 26 uORF 3′ STOP: 95 leader length: 226 |
| 1267 | AGI number: AT2G24530.1 uORF ′5 STOP: 151 uORF 3′ STOP: 217 leader length: 310 |
| 1268 | AGI number: AT2G24530.1 uORF ′5 STOP: 151 uORF 3′ STOP: 217 leader length: 310 |
| 1269 | AGI number: AT2G24530.1 uORF ′5 STOP: 62 uORF 3′ STOP: 221 leader length: 310 |
| 1270 | AGI number: AT2G24530.1 uORF ′5 STOP: 62 uORF 3′ STOP: 221 leader length: 310 |
| 1271 | AGI number: AT2G24630.1 uORF ′5 STOP: 240 uORF 3′ STOP: 294 leader length: 386 |
| 1272 | AGI number: AT2G24630.1 uORF ′5 STOP: 240 uORF 3′ STOP: 294 leader length: 386 |
| 1273 | AGI number: AT2G24630.1 uORF ′5 STOP: 233 uORF 3′ STOP: 299 leader length: 386 |
| 1274 | AGI number: AT2G24630.1 uORF ′5 STOP: 233 uORF 3′ STOP: 299 leader length: 386 |
| 1275 | AGI number: AT2G24640.2 uORF ′5 STOP: 166 uORF 3′ STOP: 358 leader length: 692 |
| 1276 | AGI number: AT2G24640.2 uORF ′5 STOP: 166 uORF 3′ STOP: 358 leader length: 692 |
| 1277 | AGI number: AT2G24945.1 uORF ′5 STOP: 1398 uORF 3′ STOP: 1452 leader length: 2331 |
| 1278 | AGI number: AT2G24945.1 uORF ′5 STOP: 1398 uORF 3′ STOP: 1452 leader length: 2331 |
| 1279 | AGI number: AT2G24945.1 uORF ′5 STOP: 210 uORF 3′ STOP: 351 leader length: 2331 |
| 1280 | AGI number: AT2G24945.1 uORF ′5 STOP: 210 uORF 3′ STOP: 351 leader length: 2331 |
| 1281 | AGI number: AT2G25190.1 uORF ′5 STOP: 35 uORF 3′ STOP: 146 leader length: 241 |
| 1282 | AGI number: AT2G25190.1 uORF ′5 STOP: 35 uORF 3′ STOP: 146 leader length: 241 |
| 1283 | AGI number: AT2G25510.1 uORF ′5 STOP: 36 uORF 3′ STOP: 363 leader length: 186 |
| 1284 | AGI number: AT2G25510.1 uORF ′5 STOP: 36 uORF 3′ STOP: 363 leader length: 186 |
| 1285 | AGI number: AT2G25700.1 uORF ′5 STOP: 51 uORF 3′ STOP: 123 leader length: 75 |
| 1286 | AGI number: AT2G25700.1 uORF ′5 STOP: 51 uORF 3′ STOP: 123 leader length: 75 |
| 1287 | AGI number: AT2G26430.3 uORF ′5 STOP: 241 uORF 3′ STOP: 298 leader length: 373 |
| 1288 | AGI number: AT2G26430.3 uORF ′5 STOP: 241 uORF 3′ STOP: 298 leader length: 373 |
| 1289 | AGI number: AT2G27030.2 uORF ′5 STOP: 52 uORF 3′ STOP: 187 leader length: 712 |
| 1290 | AGI number: AT2G27030.2 uORF ′5 STOP: 52 uORF 3′ STOP: 187 leader length: 712 |
| 1291 | AGI number: AT2G27030.2 uORF ′5 STOP: 15 uORF 3′ STOP: 174 leader length: 712 |
| 1292 | AGI number: AT2G27030.2 uORF ′5 STOP: 15 uORF 3′ STOP: 174 leader length: 712 |
| 1293 | AGI number: AT2G27060.1 uORF ′5 STOP: 73 uORF 3′ STOP: 163 leader length: 247 |
| 1294 | AGI number: AT2G27060.1 uORF ′5 STOP: 73 uORF 3′ STOP: 163 leader length: 247 |
| 1295 | AGI number: AT2G27110.2 uORF ′5 STOP: 3 uORF 3′ STOP: 81 leader length: 389 |
| 1296 | AGI number: AT2G27110.2 uORF ′5 STOP: 3 uORF 3′ STOP: 81 leader length: 389 |
| 1297 | AGI number: AT2G27228.1 uORF ′5 STOP: 85 uORF 3′ STOP: 139 leader length: 234 |
| 1298 | AGI number: AT2G27228.1 uORF ′5 STOP: 85 uORF 3′ STOP: 139 leader length: 234 |
| 1299 | AGI number: AT2G27228.1 uORF ′5 STOP: 26 uORF 3′ STOP: 158 leader length: 234 |
| 1300 | AGI number: AT2G27228.1 uORF ′5 STOP: 26 uORF 3′ STOP: 158 leader length: 234 |
| 1301 | AGI number: AT2G27230.1 uORF ′5 STOP: 85 uORF 3′ STOP: 139 leader length: 412 |
| 1302 | AGI number: AT2G27230.1 uORF ′5 STOP: 85 uORF 3′ STOP: 139 leader length: 412 |
| 1303 | AGI number: AT2G27230.1 uORF ′5 STOP: 169 uORF 3′ STOP: 340 leader length: 412 |
| 1304 | AGI number: AT2G27230.1 uORF ′5 STOP: 169 uORF 3′ STOP: 340 leader length: 412 |
| 1305 | AGI number: AT2G27230.1 uORF ′5 STOP: 26 uORF 3′ STOP: 158 leader length: 412 |
| 1306 | AGI number: AT2G27230.1 uORF ′5 STOP: 26 uORF 3′ STOP: 158 leader length: 412 |
| 1307 | AGI number: AT2G27230.2 uORF ′5 STOP: 168 uORF 3′ STOP: 339 leader length: 449 |
| 1308 | AGI number: AT2G27230.2 uORF ′5 STOP: 168 uORF 3′ STOP: 339 leader length: 449 |
| 1309 | AGI number: AT2G27230.2 uORF ′5 STOP: 296 uORF 3′ STOP: 377 leader length: 449 |
| 1310 | AGI number: AT2G27230.2 uORF ′5 STOP: 296 uORF 3′ STOP: 377 leader length: 449 |
| 1311 | AGI number: AT2G27230.2 uORF ′5 STOP: 265 uORF 3′ STOP: 367 leader length: 449 |
| 1312 | AGI number: AT2G27230.2 uORF ′5 STOP: 265 uORF 3′ STOP: 367 leader length: 449 |
| 1313 | AGI number: AT2G27230.2 uORF ′5 STOP: 25 uORF 3′ STOP: 157 leader length: 449 |
| 1314 | AGI number: AT2G27230.2 uORF ′5 STOP: 25 uORF 3′ STOP: 157 leader length: 449 |
| 1315 | AGI number: AT2G27340.2 uORF ′5 STOP: 12 uORF 3′ STOP: 150 leader length: 575 |
| 1316 | AGI number: AT2G27340.2 uORF ′5 STOP: 12 uORF 3′ STOP: 150 leader length: 575 |
| 1317 | AGI number: AT2G27340.2 uORF ′5 STOP: 536 uORF 3′ STOP: 677 leader length: 575 |
| 1318 | AGI number: AT2G27340.2 uORF ′5 STOP: 536 uORF 3′ STOP: 677 leader length: 575 |
| 1319 | AGI number: AT2G27340.4 uORF ′5 STOP: 52 uORF 3′ STOP: 184 leader length: 289 |
| 1320 | AGI number: AT2G27340.4 uORF ′5 STOP: 52 uORF 3′ STOP: 184 leader length: 289 |
| 1321 | AGI number: AT2G27350.5 uORF ′5 STOP: 8 uORF 3′ STOP: 185 leader length: 535 |
| 1322 | AGI number: AT2G27350.5 uORF ′5 STOP: 8 uORF 3′ STOP: 185 leader length: 535 |
| 1323 | AGI number: AT2G27402.1 uORF ′5 STOP: 32 uORF 3′ STOP: 95 leader length: 506 |
| 1324 | AGI number: AT2G27402.1 uORF ′5 STOP: 32 uORF 3′ STOP: 95 leader length: 506 |
| 1325 | AGI number: AT2G27830.1 uORF ′5 STOP: 155 uORF 3′ STOP: 221 leader length: 516 |
| 1326 | AGI number: AT2G27830.1 uORF ′5 STOP: 155 uORF 3′ STOP: 221 leader length: 516 |
| 1327 | AGI number: AT2G27950.1 uORF ′5 STOP: 140 uORF 3′ STOP: 299 leader length: 407 |
| 1328 | AGI number: AT2G27950.1 uORF ′5 STOP: 140 uORF 3′ STOP: 299 leader length: 407 |
| 1329 | AGI number: AT2G28105.1 uORF ′5 STOP: 295 uORF 3′ STOP: 490 leader length: 302 |
| 1330 | AGI number: AT2G28105.1 uORF ′5 STOP: 295 uORF 3′ STOP: 490 leader length: 302 |
| 1331 | AGI number: AT2G28105.1 uORF ′5 STOP: 121 uORF 3′ STOP: 181 leader length: 302 |
| 1332 | AGI number: AT2G28105.1 uORF ′5 STOP: 121 uORF 3′ STOP: 181 leader length: 302 |
| 1333 | AGI number: AT2G28320.1 uORF ′5 STOP: 98 uORF 3′ STOP: 179 leader length: 415 |
| 1334 | AGI number: AT2G28320.1 uORF ′5 STOP: 98 uORF 3′ STOP: 179 leader length: 415 |
| 1335 | AGI number: AT2G28380.1 uORF ′5 STOP: 134 uORF 3′ STOP: 200 leader length: 265 |
| 1336 | AGI number: AT2G28380.1 uORF ′5 STOP: 134 uORF 3′ STOP: 200 leader length: 265 |
| 1337 | AGI number: AT2G28490.1 uORF ′5 STOP: 5 uORF 3′ STOP: 116 leader length: 29 |
| 1338 | AGI number: AT2G28490.1 uORF ′5 STOP: 5 uORF 3′ STOP: 116 leader length: 29 |
| 1339 | AGI number: AT2G28550.2 uORF ′5 STOP: 138 uORF 3′ STOP: 210 leader length: 292 |
| 1340 | AGI number: AT2G28550.2 uORF ′5 STOP: 138 uORF 3′ STOP: 210 leader length: 292 |
| 1341 | AGI number: AT2G28810.2 uORF ′5 STOP: 188 uORF 3′ STOP: 287 leader length: 508 |
| 1342 | AGI number: AT2G28810.2 uORF ′5 STOP: 188 uORF 3′ STOP: 287 leader length: 508 |
| 1343 | AGI number: AT2G28810.2 uORF ′5 STOP: 131 uORF 3′ STOP: 191 leader length: 508 |
| 1344 | AGI number: AT2G28810.2 uORF ′5 STOP: 131 uORF 3′ STOP: 191 leader length: 508 |
| 1345 | AGI number: AT2G29190.1 uORF ′5 STOP: 110 uORF 3′ STOP: 242 leader length: 336 |
| 1346 | AGI number: AT2G29190.1 uORF ′5 STOP: 110 uORF 3′ STOP: 242 leader length: 336 |
| 1347 | AGI number: AT2G29190.1 uORF ′5 STOP: 196 uORF 3′ STOP: 253 leader length: 336 |
| 1348 | AGI number: AT2G29190.1 uORF ′5 STOP: 196 uORF 3′ STOP: 253 leader length: 336 |
| 1349 | AGI number: AT2G29190.2 uORF ′5 STOP: 179 uORF 3′ STOP: 311 leader length: 389 |
| 1350 | AGI number: AT2G29190.2 uORF ′5 STOP: 179 uORF 3′ STOP: 311 leader length: 389 |
| 1351 | AGI number: AT2G29290.1 uORF ′5 STOP: 22 uORF 3′ STOP: 184 leader length: 565 |
| 1352 | AGI number: AT2G29290.1 uORF ′5 STOP: 22 uORF 3′ STOP: 184 leader length: 565 |
| 1353 | AGI number: AT2G29290.2 uORF ′5 STOP: 158 uORF 3′ STOP: 230 leader length: 554 |
| 1354 | AGI number: AT2G29290.2 uORF ′5 STOP: 158 uORF 3′ STOP: 230 leader length: 554 |
| 1355 | AGI number: AT2G29290.2 uORF ′5 STOP: 22 uORF 3′ STOP: 184 leader length: 554 |
| 1356 | AGI number: AT2G29290.2 uORF ′5 STOP: 22 uORF 3′ STOP: 184 leader length: 554 |
| 1357 | AGI number: AT2G29600.1 uORF ′5 STOP: 168 uORF 3′ STOP: 270 leader length: 510 |
| 1358 | AGI number: AT2G29600.1 uORF ′5 STOP: 168 uORF 3′ STOP: 270 leader length: 510 |
| 1359 | AGI number: AT2G30370.1 uORF ′5 STOP: 238 uORF 3′ STOP: 397 leader length: 242 |
| 1360 | AGI number: AT2G30370.1 uORF ′5 STOP: 238 uORF 3′ STOP: 397 leader length: 242 |
| 1361 | AGI number: AT2G30370.2 uORF ′5 STOP: 111 uORF 3′ STOP: 210 leader length: 242 |
| 1362 | AGI number: AT2G30370.2 uORF ′5 STOP: 111 uORF 3′ STOP: 210 leader length: 242 |
| 1363 | AGI number: AT2G30520.2 uORF ′5 STOP: 12 uORF 3′ STOP: 138 leader length: 364 |
| 1364 | AGI number: AT2G30520.2 uORF ′5 STOP: 12 uORF 3′ STOP: 138 leader length: 364 |
| 1365 | AGI number: AT2G30520.2 uORF ′5 STOP: 59 uORF 3′ STOP: 173 leader length: 364 |
| 1366 | AGI number: AT2G30520.2 uORF ′5 STOP: 59 uORF 3′ STOP: 173 leader length: 364 |
| 1367 | AGI number: AT2G30590.1 uORF ′5 STOP: 4 uORF 3′ STOP: 154 leader length: 415 |
| 1368 | AGI number: AT2G30590.1 uORF ′5 STOP: 4 uORF 3′ STOP: 154 leader length: 415 |
| 1369 | AGI number: AT2G30590.1 uORF ′5 STOP: 264 uORF 3′ STOP: 318 leader length: 415 |
| 1370 | AGI number: AT2G30590.1 uORF ′5 STOP: 264 uORF 3′ STOP: 318 leader length: 415 |
| 1371 | AGI number: AT2G30590.1 uORF ′5 STOP: 169 uORF 3′ STOP: 331 leader length: 415 |
| 1372 | AGI number: AT2G30590.1 uORF ′5 STOP: 169 uORF 3′ STOP: 331 leader length: 415 |
| 1373 | AGI number: AT2G30590.1 uORF ′5 STOP: 26 uORF 3′ STOP: 143 leader length: 415 |
| 1374 | AGI number: AT2G30590.1 uORF ′5 STOP: 26 uORF 3′ STOP: 143 leader length: 415 |
| 1375 | AGI number: AT2G30600.5 uORF ′5 STOP: 77 uORF 3′ STOP: 140 leader length: 357 |
| 1376 | AGI number: AT2G30600.5 uORF ′5 STOP: 77 uORF 3′ STOP: 140 leader length: 357 |
| 1377 | AGI number: AT2G30600.5 uORF ′5 STOP: 70 uORF 3′ STOP: 133 leader length: 357 |
| 1378 | AGI number: AT2G30600.5 uORF ′5 STOP: 70 uORF 3′ STOP: 133 leader length: 357 |
| 1379 | AGI number: AT2G30942.1 uORF ′5 STOP: 40 uORF 3′ STOP: 289 leader length: 82 |
| 1380 | AGI number: AT2G30942.1 uORF ′5 STOP: 40 uORF 3′ STOP: 289 leader length: 82 |
| 1381 | AGI number: AT2G31010.1 uORF ′5 STOP: 2 uORF 3′ STOP: 182 leader length: 240 |
| 1382 | AGI number: AT2G31010.1 uORF ′5 STOP: 2 uORF 3′ STOP: 182 leader length: 240 |
| 1383 | AGI number: AT2G31010.2 uORF ′5 STOP: 140 uORF 3′ STOP: 215 leader length: 235 |
| 1384 | AGI number: AT2G31010.2 uORF ′5 STOP: 140 uORF 3′ STOP: 215 leader length: 235 |
| 1385 | AGI number: AT2G31010.2 uORF ′5 STOP: 76 uORF 3′ STOP: 196 leader length: 235 |
| 1386 | AGI number: AT2G31010.2 uORF ′5 STOP: 76 uORF 3′ STOP: 196 leader length: 235 |
| 1387 | AGI number: AT2G31070.1 uORF ′5 STOP: 264 uORF 3′ STOP: 363 leader length: 540 |
| 1388 | AGI number: AT2G31070.1 uORF ′5 STOP: 264 uORF 3′ STOP: 363 leader length: 540 |
| 1389 | AGI number: AT2G31280.1 uORF ′5 STOP: 311 uORF 3′ STOP: 449 leader length: 591 |
| 1390 | AGI number: AT2G31280.1 uORF ′5 STOP: 311 uORF 3′ STOP: 449 leader length: 591 |
| 1391 | AGI number: AT2G31280.1 uORF ′5 STOP: 370 uORF 3′ STOP: 445 leader length: 591 |
| 1392 | AGI number: AT2G31280.1 uORF ′5 STOP: 370 uORF 3′ STOP: 445 leader length: 591 |
| 1393 | AGI number: AT2G31280.3 uORF ′5 STOP: 370 uORF 3′ STOP: 445 leader length: 591 |
| 1394 | AGI number: AT2G31280.3 uORF ′5 STOP: 370 uORF 3′ STOP: 445 leader length: 591 |
| 1395 | AGI number: AT2G31280.3 uORF ′5 STOP: 311 uORF 3′ STOP: 449 leader length: 591 |
| 1396 | AGI number: AT2G31280.3 uORF ′5 STOP: 311 uORF 3′ STOP: 449 leader length: 591 |
| 1397 | AGI number: AT2G31490.1 uORF ′5 STOP: 67 uORF 3′ STOP: 340 leader length: 77 |
| 1398 | AGI number: AT2G31490.1 uORF ′5 STOP: 67 uORF 3′ STOP: 340 leader length: 77 |
| 1399 | AGI number: AT2G32190.2 uORF ′5 STOP: 24 uORF 3′ STOP: 243 leader length: 90 |
| 1400 | AGI number: AT2G32190.2 uORF ′5 STOP: 24 uORF 3′ STOP: 243 leader length: 90 |
| 1401 | AGI number: AT2G32235.1 uORF ′5 STOP: 521 uORF 3′ STOP: 584 leader length: 957 |
| 1402 | AGI number: AT2G32235.1 uORF ′5 STOP: 521 uORF 3′ STOP: 584 leader length: 957 |
| 1403 | AGI number: AT2G32235.1 uORF ′5 STOP: 581 uORF 3′ STOP: 782 leader length: 957 |
| 1404 | AGI number: AT2G32235.1 uORF ′5 STOP: 581 uORF 3′ STOP: 782 leader length: 957 |
| 1405 | AGI number: AT2G32235.1 uORF ′5 STOP: 646 uORF 3′ STOP: 787 leader length: 957 |
| 1406 | AGI number: AT2G32235.1 uORF ′5 STOP: 646 uORF 3′ STOP: 787 leader length: 957 |
| 1407 | AGI number: AT2G32250.1 uORF ′5 STOP: 376 uORF 3′ STOP: 433 leader length: 413 |
| 1408 | AGI number: AT2G32250.1 uORF ′5 STOP: 376 uORF 3′ STOP: 433 leader length: 413 |
| 1409 | AGI number: AT2G32250.2 uORF ′5 STOP: 251 uORF 3′ STOP: 305 leader length: 413 |
| 1410 | AGI number: AT2G32250.2 uORF ′5 STOP: 251 uORF 3′ STOP: 305 leader length: 413 |
| 1411 | AGI number: AT2G32250.2 uORF ′5 STOP: 240 uORF 3′ STOP: 297 leader length: 413 |
| 1412 | AGI number: AT2G32250.2 uORF ′5 STOP: 240 uORF 3′ STOP: 297 leader length: 413 |
| 1413 | AGI number: AT2G32250.4 uORF ′5 STOP: 52 uORF 3′ STOP: 133 leader length: 413 |
| 1414 | AGI number: AT2G32250.4 uORF ′5 STOP: 52 uORF 3′ STOP: 133 leader length: 413 |
| 1415 | AGI number: AT2G32320.2 uORF ′5 STOP: 93 uORF 3′ STOP: 165 leader length: 212 |
| 1416 | AGI number: AT2G32320.2 uORF ′5 STOP: 93 uORF 3′ STOP: 165 leader length: 212 |
| 1417 | AGI number: AT2G32430.1 uORF ′5 STOP: 171 uORF 3′ STOP: 246 leader length: 330 |
| 1418 | AGI number: AT2G32430.1 uORF ′5 STOP: 171 uORF 3′ STOP: 246 leader length: 330 |
| 1419 | AGI number: AT2G32440.2 uORF ′5 STOP: 81 uORF 3′ STOP: 165 leader length: 240 |
| 1420 | AGI number: AT2G32440.2 uORF ′5 STOP: 81 uORF 3′ STOP: 165 leader length: 240 |
| 1421 | AGI number: AT2G32440.2 uORF ′5 STOP: 109 uORF 3′ STOP: 160 leader length: 240 |
| 1422 | AGI number: AT2G32440.2 uORF ′5 STOP: 109 uORF 3′ STOP: 160 leader length: 240 |
| 1423 | AGI number: AT2G32890.1 uORF ′5 STOP: 751 uORF 3′ STOP: 865 leader length: 824 |
| 1424 | AGI number: AT2G32890.1 uORF ′5 STOP: 751 uORF 3′ STOP: 865 leader length: 824 |
| 1425 | AGI number: AT2G32890.1 uORF ′5 STOP: 179 uORF 3′ STOP: 356 leader length: 824 |
| 1426 | AGI number: AT2G32890.1 uORF ′5 STOP: 179 uORF 3′ STOP: 356 leader length: 824 |
| 1427 | AGI number: AT2G32890.1 uORF ′5 STOP: 198 uORF 3′ STOP: 279 leader length: 824 |
| 1428 | AGI number: AT2G32890.1 uORF ′5 STOP: 198 uORF 3′ STOP: 279 leader length: 824 |
| 1429 | AGI number: AT2G33170.1 uORF ′5 STOP: 261 uORF 3′ STOP: 315 leader length: 516 |
| 1430 | AGI number: AT2G33170.1 uORF ′5 STOP: 261 uORF 3′ STOP: 315 leader length: 516 |
| 1431 | AGI number: AT2G33170.1 uORF ′5 STOP: 55 uORF 3′ STOP: 136 leader length: 516 |
| 1432 | AGI number: AT2G33170.1 uORF ′5 STOP: 55 uORF 3′ STOP: 136 leader length: 516 |
| 1433 | AGI number: AT2G33390.1 uORF ′5 STOP: 284 uORF 3′ STOP: 338 leader length: 423 |
| 1434 | AGI number: AT2G33390.1 uORF ′5 STOP: 284 uORF 3′ STOP: 338 leader length: 423 |
| 1435 | AGI number: AT2G33700.1 uORF ′5 STOP: 78 uORF 3′ STOP: 210 leader length: 776 |
| 1436 | AGI number: AT2G33700.1 uORF ′5 STOP: 78 uORF 3′ STOP: 210 leader length: 776 |
| 1437 | AGI number: AT2G33770.1 uORF ′5 STOP: 86 uORF 3′ STOP: 275 leader length: 1152 |
| 1438 | AGI number: AT2G33770.1 uORF ′5 STOP: 86 uORF 3′ STOP: 275 leader length: 1152 |
| 1439 | AGI number: AT2G33770.1 uORF ′5 STOP: 272 uORF 3′ STOP: 329 leader length: 1152 |
| 1440 | AGI number: AT2G33770.1 uORF ′5 STOP: 272 uORF 3′ STOP: 329 leader length: 1152 |
| 1441 | AGI number: AT2G34260.2 uORF ′5 STOP: 3 uORF 3′ STOP: 231 leader length: 275 |
| 1442 | AGI number: AT2G34260.2 uORF ′5 STOP: 3 uORF 3′ STOP: 231 leader length: 275 |
| 1443 | AGI number: AT2G34660.1 uORF ′5 STOP: 134 uORF 3′ STOP: 260 leader length: 567 |
| 1444 | AGI number: AT2G34660.1 uORF ′5 STOP: 134 uORF 3′ STOP: 260 leader length: 567 |
| 1445 | AGI number: AT2G34670.1 uORF ′5 STOP: 299 uORF 3′ STOP: 395 leader length: 502 |
| 1446 | AGI number: AT2G34670.1 uORF ′5 STOP: 299 uORF 3′ STOP: 395 leader length: 502 |
| 1447 | AGI number: AT2G34670.1 uORF ′5 STOP: 202 uORF 3′ STOP: 403 leader length: 502 |
| 1448 | AGI number: AT2G34670.1 uORF ′5 STOP: 202 uORF 3′ STOP: 403 leader length: 502 |
| 1449 | AGI number: AT2G34670.1 uORF ′5 STOP: 197 uORF 3′ STOP: 266 leader length: 502 |
| 1450 | AGI number: AT2G34670.1 uORF ′5 STOP: 197 uORF 3′ STOP: 266 leader length: 502 |
| 1451 | AGI number: AT2G34670.2 uORF ′5 STOP: 361 uORF 3′ STOP: 457 leader length: 564 |
| 1452 | AGI number: AT2G34670.2 uORF ′5 STOP: 361 uORF 3′ STOP: 457 leader length: 564 |
| 1453 | AGI number: AT2G34670.2 uORF ′5 STOP: 132 uORF 3′ STOP: 267 leader length: 564 |
| 1454 | AGI number: AT2G34670.2 uORF ′5 STOP: 132 uORF 3′ STOP: 267 leader length: 564 |
| 1455 | AGI number: AT2G34670.2 uORF ′5 STOP: 264 uORF 3′ STOP: 465 leader length: 564 |
| 1456 | AGI number: AT2G34670.2 uORF ′5 STOP: 264 uORF 3′ STOP: 465 leader length: 564 |
| 1457 | AGI number: AT2G34780.2 uORF ′5 STOP: 159 uORF 3′ STOP: 240 leader length: 529 |
| 1458 | AGI number: AT2G34780.2 uORF ′5 STOP: 159 uORF 3′ STOP: 240 leader length: 529 |
| 1459 | AGI number: AT2G35155.1 uORF ′5 STOP: 46 uORF 3′ STOP: 262 leader length: 308 |
| 1460 | AGI number: AT2G35155.1 uORF ′5 STOP: 46 uORF 3′ STOP: 262 leader length: 308 |
| 1461 | AGI number: AT2G35310.1 uORF ′5 STOP: 69 uORF 3′ STOP: 189 leader length: 88 |
| 1462 | AGI number: AT2G35310.1 uORF ′5 STOP: 69 uORF 3′ STOP: 189 leader length: 88 |
| 1463 | AGI number: AT2G35390.1 uORF ′5 STOP: 243 uORF 3′ STOP: 339 leader length: 673 |
| 1464 | AGI number: AT2G35390.1 uORF ′5 STOP: 243 uORF 3′ STOP: 339 leader length: 673 |
| 1465 | AGI number: AT2G35510.1 uORF ′5 STOP: 270 uORF 3′ STOP: 330 leader length: 417 |
| 1466 | AGI number: AT2G35510.1 uORF ′5 STOP: 270 uORF 3′ STOP: 330 leader length: 417 |
| 1467 | AGI number: AT2G35510.1 uORF ′5 STOP: 251 uORF 3′ STOP: 344 leader length: 417 |
| 1468 | AGI number: AT2G35510.1 uORF ′5 STOP: 251 uORF 3′ STOP: 344 leader length: 417 |
| 1469 | AGI number: AT2G35510.1 uORF ′5 STOP: 50 uORF 3′ STOP: 122 leader length: 417 |
| 1470 | AGI number: AT2G35510.1 uORF ′5 STOP: 50 uORF 3′ STOP: 122 leader length: 417 |
| 1471 | AGI number: AT2G35600.1 uORF ′5 STOP: 90 uORF 3′ STOP: 219 leader length: 376 |
| 1472 | AGI number: AT2G35600.1 uORF ′5 STOP: 90 uORF 3′ STOP: 219 leader length: 376 |
| 1473 | AGI number: AT2G35660.3 uORF ′5 STOP: 142 uORF 3′ STOP: 310 leader length: 446 |
| 1474 | AGI number: AT2G35660.3 uORF ′5 STOP: 142 uORF 3′ STOP: 310 leader length: 446 |
| 1475 | AGI number: AT2G35680.1 uORF ′5 STOP: 173 uORF 3′ STOP: 371 leader length: 495 |
| 1476 | AGI number: AT2G35680.1 uORF ′5 STOP: 173 uORF 3′ STOP: 371 leader length: 495 |
| 1477 | AGI number: AT2G35750.1 uORF ′5 STOP: 168 uORF 3′ STOP: 219 leader length: 201 |
| 1478 | AGI number: AT2G35750.1 uORF ′5 STOP: 168 uORF 3′ STOP: 219 leader length: 201 |
| 1479 | AGI number: AT2G35750.1 uORF ′5 STOP: 179 uORF 3′ STOP: 230 leader length: 201 |
| 1480 | AGI number: AT2G35750.1 uORF ′5 STOP: 179 uORF 3′ STOP: 230 leader length: 201 |
| 1481 | AGI number: AT2G35800.1 uORF ′5 STOP: 168 uORF 3′ STOP: 222 leader length: 392 |
| 1482 | AGI number: AT2G35800.1 uORF ′5 STOP: 168 uORF 3′ STOP: 222 leader length: 392 |
| 1483 | AGI number: AT2G35800.1 uORF ′5 STOP: 1 uORF 3′ STOP: 202 leader length: 392 |
| 1484 | AGI number: AT2G35800.1 uORF ′5 STOP: 1 uORF 3′ STOP: 202 leader length: 392 |
| 1485 | AGI number: AT2G35880.1 uORF ′5 STOP: 167 uORF 3′ STOP: 227 leader length: 309 |
| 1486 | AGI number: AT2G35880.1 uORF ′5 STOP: 167 uORF 3′ STOP: 227 leader length: 309 |
| 1487 | AGI number: AT2G35880.1 uORF ′5 STOP: 159 uORF 3′ STOP: 222 leader length: 309 |
| 1488 | AGI number: AT2G35880.1 uORF ′5 STOP: 159 uORF 3′ STOP: 222 leader length: 309 |
| 1489 | AGI number: AT2G35940.3 uORF ′5 STOP: 141 uORF 3′ STOP: 195 leader length: 543 |
| 1490 | AGI number: AT2G35940.3 uORF ′5 STOP: 141 uORF 3′ STOP: 195 leader length: 543 |
| 1491 | AGI number: AT2G36210.1 uORF ′5 STOP: 32 uORF 3′ STOP: 179 leader length: 71 |
| 1492 | AGI number: AT2G36210.1 uORF ′5 STOP: 32 uORF 3′ STOP: 179 leader length: 71 |
| 1493 | AGI number: AT2G36370.1 uORF ′5 STOP: 16 uORF 3′ STOP: 133 leader length: 101 |
| 1494 | AGI number: AT2G36370.1 uORF ′5 STOP: 16 uORF 3′ STOP: 133 leader length: 101 |
| 1495 | AGI number: AT2G36460.2 uORF ′5 STOP: 295 uORF 3′ STOP: 355 leader length: 485 |
| 1496 | AGI number: AT2G36460.2 uORF ′5 STOP: 295 uORF 3′ STOP: 355 leader length: 485 |
| 1497 | AGI number: AT2G36470.1 uORF ′5 STOP: 149 uORF 3′ STOP: 305 leader length: 622 |
| 1498 | AGI number: AT2G36470.1 uORF ′5 STOP: 149 uORF 3′ STOP: 305 leader length: 622 |
| 1499 | AGI number: AT2G36485.1 uORF ′5 STOP: 36 uORF 3′ STOP: 156 leader length: 259 |
| 1500 | AGI number: AT2G36485.1 uORF ′5 STOP: 36 uORF 3′ STOP: 156 leader length: 259 |
| 1501 | AGI number: AT2G36720.1 uORF ′5 STOP: 120 uORF 3′ STOP: 180 leader length: 376 |
| 1502 | AGI number: AT2G36720.1 uORF ′5 STOP: 120 uORF 3′ STOP: 180 leader length: 376 |
| 1503 | AGI number: AT2G36720.1 uORF ′5 STOP: 133 uORF 3′ STOP: 199 leader length: 376 |
| 1504 | AGI number: AT2G36720.1 uORF ′5 STOP: 133 uORF 3′ STOP: 199 leader length: 376 |
| 1505 | AGI number: AT2G36840.1 uORF ′5 STOP: 7 uORF 3′ STOP: 85 leader length: 164 |
| 1506 | AGI number: AT2G36840.1 uORF ′5 STOP: 7 uORF 3′ STOP: 85 leader length: 164 |
| 1507 | AGI number: AT2G36960.2 uORF ′5 STOP: 185 uORF 3′ STOP: 248 leader length: 201 |
| 1508 | AGI number: AT2G36960.2 uORF ′5 STOP: 185 uORF 3′ STOP: 248 leader length: 201 |
| 1509 | AGI number: AT2G37080.1 uORF ′5 STOP: 54 uORF 3′ STOP: 138 leader length: 295 |
| 1510 | AGI number: AT2G37080.1 uORF ′5 STOP: 54 uORF 3′ STOP: 138 leader length: 295 |
| 1511 | AGI number: AT2G37080.1 uORF ′5 STOP: 68 uORF 3′ STOP: 143 leader length: 295 |
| 1512 | AGI number: AT2G37080.1 uORF ′5 STOP: 68 uORF 3′ STOP: 143 leader length: 295 |
| 1513 | AGI number: AT2G37150.2 uORF ′5 STOP: 161 uORF 3′ STOP: 230 leader length: 666 |
| 1514 | AGI number: AT2G37150.2 uORF ′5 STOP: 161 uORF 3′ STOP: 230 leader length: 666 |
| 1515 | AGI number: AT2G37150.2 uORF ′5 STOP: 230 uORF 3′ STOP: 320 leader length: 666 |
| 1516 | AGI number: AT2G37150.2 uORF ′5 STOP: 230 uORF 3′ STOP: 320 leader length: 666 |
| 1517 | AGI number: AT2G37150.2 uORF ′5 STOP: 184 uORF 3′ STOP: 259 leader length: 666 |
| 1518 | AGI number: AT2G37150.2 uORF ′5 STOP: 184 uORF 3′ STOP: 259 leader length: 666 |
| 1519 | AGI number: AT2G37150.2 uORF ′5 STOP: 526 uORF 3′ STOP: 589 leader length: 666 |
| 1520 | AGI number: AT2G37150.2 uORF ′5 STOP: 526 uORF 3′ STOP: 589 leader length: 666 |
| 1521 | AGI number: AT2G37150.2 uORF ′5 STOP: 472 uORF 3′ STOP: 529 leader length: 666 |
| 1522 | AGI number: AT2G37150.2 uORF ′5 STOP: 472 uORF 3′ STOP: 529 leader length: 666 |
| 1523 | AGI number: AT2G37160.2 uORF ′5 STOP: 19 uORF 3′ STOP: 133 leader length: 219 |
| 1524 | AGI number: AT2G37160.2 uORF ′5 STOP: 19 uORF 3′ STOP: 133 leader length: 219 |
| 1525 | AGI number: AT2G37340.2 uORF ′5 STOP: 35 uORF 3′ STOP: 251 leader length: 312 |
| 1526 | AGI number: AT2G37340.2 uORF ′5 STOP: 35 uORF 3′ STOP: 251 leader length: 312 |
| 1527 | AGI number: AT2G37340.2 uORF ′5 STOP: 78 uORF 3′ STOP: 210 leader length: 312 |
| 1528 | AGI number: AT2G37340.2 uORF ′5 STOP: 78 uORF 3′ STOP: 210 leader length: 312 |
| 1529 | AGI number: AT2G37340.2 uORF ′5 STOP: 175 uORF 3′ STOP: 238 leader length: 312 |
| 1530 | AGI number: AT2G37340.2 uORF ′5 STOP: 175 uORF 3′ STOP: 238 leader length: 312 |
| 1531 | AGI number: AT2G37340.3 uORF ′5 STOP: 97 uORF 3′ STOP: 151 leader length: 423 |
| 1532 | AGI number: AT2G37340.3 uORF ′5 STOP: 97 uORF 3′ STOP: 151 leader length: 423 |
| 1533 | AGI number: AT2G37340.3 uORF ′5 STOP: 78 uORF 3′ STOP: 210 leader length: 423 |
| 1534 | AGI number: AT2G37340.3 uORF ′5 STOP: 78 uORF 3′ STOP: 210 leader length: 423 |
| 1535 | AGI number: AT2G37480.1 uORF ′5 STOP: 268 uORF 3′ STOP: 430 leader length: 601 |
| 1536 | AGI number: AT2G37480.1 uORF ′5 STOP: 268 uORF 3′ STOP: 430 leader length: 601 |
| 1537 | AGI number: AT2G37480.2 uORF ′5 STOP: 268 uORF 3′ STOP: 430 leader length: 601 |
| 1538 | AGI number: AT2G37480.2 uORF ′5 STOP: 268 uORF 3′ STOP: 430 leader length: 601 |
| 1539 | AGI number: AT2G37570.1 uORF ′5 STOP: 57 uORF 3′ STOP: 120 leader length: 423 |
| 1540 | AGI number: AT2G37570.1 uORF ′5 STOP: 57 uORF 3′ STOP: 120 leader length: 423 |
| 1541 | AGI number: AT2G37570.2 uORF ′5 STOP: 67 uORF 3′ STOP: 130 leader length: 435 |
| 1542 | AGI number: AT2G37570.2 uORF ′5 STOP: 67 uORF 3′ STOP: 130 leader length: 435 |
| 1543 | AGI number: AT2G37840.2 uORF ′5 STOP: 800 uORF 3′ STOP: 857 leader length: 871 |
| 1544 | AGI number: AT2G37840.2 uORF ′5 STOP: 800 uORF 3′ STOP: 857 leader length: 871 |
| 1545 | AGI number: AT2G37840.2 uORF ′5 STOP: 724 uORF 3′ STOP: 871 leader length: 871 |
| 1546 | AGI number: AT2G37840.2 uORF ′5 STOP: 724 uORF 3′ STOP: 871 leader length: 871 |
| 1547 | AGI number: AT2G37890.1 uORF ′5 STOP: 96 uORF 3′ STOP: 165 leader length: 160 |
| 1548 | AGI number: AT2G37890.1 uORF ′5 STOP: 96 uORF 3′ STOP: 165 leader length: 160 |
| 1549 | AGI number: AT2G38040.2 uORF ′5 STOP: 28 uORF 3′ STOP: 175 leader length: 291 |
| 1550 | AGI number: AT2G38040.2 uORF ′5 STOP: 28 uORF 3′ STOP: 175 leader length: 291 |
| 1551 | AGI number: AT2G38290.2 uORF ′5 STOP: 131 uORF 3′ STOP: 392 leader length: 519 |
| 1552 | AGI number: AT2G38290.2 uORF ′5 STOP: 131 uORF 3′ STOP: 392 leader length: 519 |
| 1553 | AGI number: AT2G38290.2 uORF ′5 STOP: 250 uORF 3′ STOP: 412 leader length: 519 |
| 1554 | AGI number: AT2G38290.2 uORF ′5 STOP: 250 uORF 3′ STOP: 412 leader length: 519 |
| 1555 | AGI number: AT2G38820.1 uORF ′5 STOP: 122 uORF 3′ STOP: 179 leader length: 368 |
| 1556 | AGI number: AT2G38820.1 uORF ′5 STOP: 122 uORF 3′ STOP: 179 leader length: 368 |
| 1557 | AGI number: AT2G38820.1 uORF ′5 STOP: 130 uORF 3′ STOP: 187 leader length: 368 |
| 1558 | AGI number: AT2G38820.1 uORF ′5 STOP: 130 uORF 3′ STOP: 187 leader length: 368 |
| 1559 | AGI number: AT2G38820.1 uORF ′5 STOP: 108 uORF 3′ STOP: 168 leader length: 368 |
| 1560 | AGI number: AT2G38820.1 uORF ′5 STOP: 108 uORF 3′ STOP: 168 leader length: 368 |
| 1561 | AGI number: AT2G38820.1 uORF ′5 STOP: 68 uORF 3′ STOP: 125 leader length: 368 |
| 1562 | AGI number: AT2G38820.1 uORF ′5 STOP: 68 uORF 3′ STOP: 125 leader length: 368 |
| 1563 | AGI number: AT2G38820.2 uORF ′5 STOP: 135 uORF 3′ STOP: 192 leader length: 435 |
| 1564 | AGI number: AT2G38820.2 uORF ′5 STOP: 135 uORF 3′ STOP: 192 leader length: 435 |
| 1565 | AGI number: AT2G38820.2 uORF ′5 STOP: 103 uORF 3′ STOP: 163 leader length: 435 |
| 1566 | AGI number: AT2G38820.2 uORF ′5 STOP: 103 uORF 3′ STOP: 163 leader length: 435 |
| 1567 | AGI number: AT2G39280.2 uORF ′5 STOP: 99 uORF 3′ STOP: 228 leader length: 204 |
| 1568 | AGI number: AT2G39280.2 uORF ′5 STOP: 99 uORF 3′ STOP: 228 leader length: 204 |
| 1569 | AGI number: AT2G39970.1 uORF ′5 STOP: 55 uORF 3′ STOP: 139 leader length: 225 |
| 1570 | AGI number: AT2G39970.1 uORF ′5 STOP: 55 uORF 3′ STOP: 139 leader length: 225 |
| 1571 | AGI number: AT2G40120.1 uORF ′5 STOP: 187 uORF 3′ STOP: 253 leader length: 334 |
| 1572 | AGI number: AT2G40120.1 uORF ′5 STOP: 187 uORF 3′ STOP: 253 leader length: 334 |
| 1573 | AGI number: AT2G40460.1 uORF ′5 STOP: 24 uORF 3′ STOP: 87 leader length: 216 |
| 1574 | AGI number: AT2G40460.1 uORF ′5 STOP: 24 uORF 3′ STOP: 87 leader length: 216 |
| 1575 | AGI number: AT2G41100.3 uORF ′5 STOP: 70 uORF 3′ STOP: 127 leader length: 257 |
| 1576 | AGI number: AT2G41100.3 uORF ′5 STOP: 70 uORF 3′ STOP: 127 leader length: 257 |
| 1577 | AGI number: AT2G41430.1 uORF ′5 STOP: 106 uORF 3′ STOP: 184 leader length: 275 |
| 1578 | AGI number: AT2G41430.1 uORF ′5 STOP: 106 uORF 3′ STOP: 184 leader length: 275 |
| 1579 | AGI number: AT2G41430.4 uORF ′5 STOP: 220 uORF 3′ STOP: 277 leader length: 504 |
| 1580 | AGI number: AT2G41430.4 uORF ′5 STOP: 220 uORF 3′ STOP: 277 leader length: 504 |
| 1581 | AGI number: AT2G41430.4 uORF ′5 STOP: 106 uORF 3′ STOP: 184 leader length: 504 |
| 1582 | AGI number: AT2G41430.4 uORF ′5 STOP: 106 uORF 3′ STOP: 184 leader length: 504 |
| 1583 | AGI number: AT2G41430.5 uORF ′5 STOP: 106 uORF 3′ STOP: 184 leader length: 294 |
| 1584 | AGI number: AT2G41430.5 uORF ′5 STOP: 106 uORF 3′ STOP: 184 leader length: 294 |
| 1585 | AGI number: AT2G41440.1 uORF ′5 STOP: 7 uORF 3′ STOP: 85 leader length: 534 |
| 1586 | AGI number: AT2G41440.1 uORF ′5 STOP: 7 uORF 3′ STOP: 85 leader length: 534 |
| 1587 | AGI number: AT2G41710.3 uORF ′5 STOP: 29 uORF 3′ STOP: 413 leader length: 147 |
| 1588 | AGI number: AT2G41710.3 uORF ′5 STOP: 29 uORF 3′ STOP: 413 leader length: 147 |
| 1589 | AGI number: AT2G41830.1 uORF ′5 STOP: 73 uORF 3′ STOP: 142 leader length: 430 |
| 1590 | AGI number: AT2G41830.1 uORF ′5 STOP: 73 uORF 3′ STOP: 142 leader length: 430 |
| 1591 | AGI number: AT2G42030.1 uORF ′5 STOP: 7 uORF 3′ STOP: 187 leader length: 375 |
| 1592 | AGI number: AT2G42030.1 uORF ′5 STOP: 7 uORF 3′ STOP: 187 leader length: 375 |
| 1593 | AGI number: AT2G42030.1 uORF ′5 STOP: 86 uORF 3′ STOP: 167 leader length: 375 |
| 1594 | AGI number: AT2G42030.1 uORF ′5 STOP: 86 uORF 3′ STOP: 167 leader length: 375 |
| 1595 | AGI number: AT2G42300.1 uORF ′5 STOP: 29 uORF 3′ STOP: 101 leader length: 386 |
| 1596 | AGI number: AT2G42300.1 uORF ′5 STOP: 29 uORF 3′ STOP: 101 leader length: 386 |
| 1597 | AGI number: AT2G42300.1 uORF ′5 STOP: 9 uORF 3′ STOP: 90 leader length: 386 |
| 1598 | AGI number: AT2G42300.1 uORF ′5 STOP: 9 uORF 3′ STOP: 90 leader length: 386 |
| 1599 | AGI number: AT2G42300.2 uORF ′5 STOP: 28 uORF 3′ STOP: 100 leader length: 385 |
| 1600 | AGI number: AT2G42300.2 uORF ′5 STOP: 28 uORF 3′ STOP: 100 leader length: 385 |
| 1601 | AGI number: AT2G42300.2 uORF ′5 STOP: 8 uORF 3′ STOP: 89 leader length: 385 |
| 1602 | AGI number: AT2G42300.2 uORF ′5 STOP: 8 uORF 3′ STOP: 89 leader length: 385 |
| 1603 | AGI number: AT2G42320.2 uORF ′5 STOP: 79 uORF 3′ STOP: 217 leader length: 346 |
| 1604 | AGI number: AT2G42320.2 uORF ′5 STOP: 79 uORF 3′ STOP: 217 leader length: 346 |
| 1605 | AGI number: AT2G42380.1 uORF ′5 STOP: 83 uORF 3′ STOP: 173 leader length: 285 |
| 1606 | AGI number: AT2G42380.1 uORF ′5 STOP: 83 uORF 3′ STOP: 173 leader length: 285 |
| 1607 | AGI number: AT2G42380.1 uORF ′5 STOP: 138 uORF 3′ STOP: 192 leader length: 285 |
| 1608 | AGI number: AT2G42380.1 uORF ′5 STOP: 138 uORF 3′ STOP: 192 leader length: 285 |
| 1609 | AGI number: AT2G42380.2 uORF ′5 STOP: 83 uORF 3′ STOP: 173 leader length: 285 |
| 1610 | AGI number: AT2G42380.2 uORF ′5 STOP: 83 uORF 3′ STOP: 173 leader length: 285 |
| 1611 | AGI number: AT2G42880.1 uORF ′5 STOP: 225 uORF 3′ STOP: 342 leader length: 433 |
| 1612 | AGI number: AT2G42880.1 uORF ′5 STOP: 225 uORF 3′ STOP: 342 leader length: 433 |
| 1613 | AGI number: AT2G43320.1 uORF ′5 STOP: 41 uORF 3′ STOP: 155 leader length: 311 |
| 1614 | AGI number: AT2G43320.1 uORF ′5 STOP: 41 uORF 3′ STOP: 155 leader length: 311 |
| 1615 | AGI number: AT2G43430.2 uORF ′5 STOP: 160 uORF 3′ STOP: 253 leader length: 624 |
| 1616 | AGI number: AT2G43430.2 uORF ′5 STOP: 160 uORF 3′ STOP: 253 leader length: 624 |
| 1617 | AGI number: AT2G43430.2 uORF ′5 STOP: 185 uORF 3′ STOP: 260 leader length: 624 |
| 1618 | AGI number: AT2G43430.2 uORF ′5 STOP: 185 uORF 3′ STOP: 260 leader length: 624 |
| 1619 | AGI number: AT2G43745.1 uORF ′5 STOP: 129 uORF 3′ STOP: 252 leader length: 624 |
| 1620 | AGI number: AT2G43745.1 uORF ′5 STOP: 129 uORF 3′ STOP: 252 leader length: 624 |
| 1621 | AGI number: AT2G43745.1 uORF ′5 STOP: 121 uORF 3′ STOP: 283 leader length: 624 |
| 1622 | AGI number: AT2G43745.1 uORF ′5 STOP: 121 uORF 3′ STOP: 283 leader length: 624 |
| 1623 | AGI number: AT2G43745.1 uORF ′5 STOP: 89 uORF 3′ STOP: 239 leader length: 624 |
| 1624 | AGI number: AT2G43745.1 uORF ′5 STOP: 89 uORF 3′ STOP: 239 leader length: 624 |
| 1625 | AGI number: AT2G44090.1 uORF ′5 STOP: 57 uORF 3′ STOP: 198 leader length: 465 |
| 1626 | AGI number: AT2G44090.1 uORF ′5 STOP: 57 uORF 3′ STOP: 198 leader length: 465 |
| 1627 | AGI number: AT2G44090.2 uORF ′5 STOP: 10 uORF 3′ STOP: 112 leader length: 365 |
| 1628 | AGI number: AT2G44090.2 uORF ′5 STOP: 10 uORF 3′ STOP: 112 leader length: 365 |
| 1629 | AGI number: AT2G44270.2 uORF ′5 STOP: 105 uORF 3′ STOP: 204 leader length: 120 |
| 1630 | AGI number: AT2G44270.2 uORF ′5 STOP: 105 uORF 3′ STOP: 204 leader length: 120 |
| 1631 | AGI number: AT2G44410.1 uORF ′5 STOP: 69 uORF 3′ STOP: 222 leader length: 310 |
| 1632 | AGI number: AT2G44410.1 uORF ′5 STOP: 69 uORF 3′ STOP: 222 leader length: 310 |
| 1633 | AGI number: AT2G44410.1 uORF ′5 STOP: 142 uORF 3′ STOP: 229 leader length: 310 |
| 1634 | AGI number: AT2G44410.1 uORF ′5 STOP: 142 uORF 3′ STOP: 229 leader length: 310 |
| 1635 | AGI number: AT2G44650.1 uORF ′5 STOP: 183 uORF 3′ STOP: 291 leader length: 600 |
| 1636 | AGI number: AT2G44650.1 uORF ′5 STOP: 183 uORF 3′ STOP: 291 leader length: 600 |
| 1637 | AGI number: AT2G44650.1 uORF ′5 STOP: 209 uORF 3′ STOP: 275 leader length: 600 |
| 1638 | AGI number: AT2G44650.1 uORF ′5 STOP: 209 uORF 3′ STOP: 275 leader length: 600 |
| 1639 | AGI number: AT2G44670.1 uORF ′5 STOP: 89 uORF 3′ STOP: 434 leader length: 99 |
| 1640 | AGI number: AT2G44670.1 uORF ′5 STOP: 89 uORF 3′ STOP: 434 leader length: 99 |
| 1641 | AGI number: AT2G44850.2 uORF ′5 STOP: 255 uORF 3′ STOP: 399 leader length: 441 |
| 1642 | AGI number: AT2G44850.2 uORF ′5 STOP: 255 uORF 3′ STOP: 399 leader length: 441 |
| 1643 | AGI number: AT2G44850.2 uORF ′5 STOP: 271 uORF 3′ STOP: 328 leader length: 441 |
| 1644 | AGI number: AT2G44850.2 uORF ′5 STOP: 271 uORF 3′ STOP: 328 leader length: 441 |
| 1645 | AGI number: AT2G44900.1 uORF ′5 STOP: 290 uORF 3′ STOP: 419 leader length: 534 |
| 1646 | AGI number: AT2G44900.1 uORF ′5 STOP: 290 uORF 3′ STOP: 419 leader length: 534 |
| 1647 | AGI number: AT2G45010.2 uORF ′5 STOP: 111 uORF 3′ STOP: 243 leader length: 859 |
| 1648 | AGI number: AT2G45010.2 uORF ′5 STOP: 111 uORF 3′ STOP: 243 leader length: 859 |
| 1649 | AGI number: AT2G45380.2 uORF ′5 STOP: 574 uORF 3′ STOP: 628 leader length: 1339 |
| 1650 | AGI number: AT2G45380.2 uORF ′5 STOP: 574 uORF 3′ STOP: 628 leader length: 1339 |
| 1651 | AGI number: AT2G45380.2 uORF ′5 STOP: 218 uORF 3′ STOP: 272 leader length: 1339 |
| 1652 | AGI number: AT2G45380.2 uORF ′5 STOP: 218 uORF 3′ STOP: 272 leader length: 1339 |
| 1653 | AGI number: AT2G45380.2 uORF ′5 STOP: 710 uORF 3′ STOP: 815 leader length: 1339 |
| 1654 | AGI number: AT2G45380.2 uORF ′5 STOP: 710 uORF 3′ STOP: 815 leader length: 1339 |
| 1655 | AGI number: AT2G45380.2 uORF ′5 STOP: 943 uORF 3′ STOP: 1315 leader length: 1339 |
| 1656 | AGI number: AT2G45380.2 uORF ′5 STOP: 943 uORF 3′ STOP: 1315 leader length: 1339 |
| 1657 | AGI number: AT2G45460.2 uORF ′5 STOP: 150 uORF 3′ STOP: 201 leader length: 151 |
| 1658 | AGI number: AT2G45460.2 uORF ′5 STOP: 150 uORF 3′ STOP: 201 leader length: 151 |
| 1659 | AGI number: AT2G45600.1 uORF ′5 STOP: 7 uORF 3′ STOP: 88 leader length: 211 |
| 1660 | AGI number: AT2G45600.1 uORF ′5 STOP: 7 uORF 3′ STOP: 88 leader length: 211 |
| 1661 | AGI number: AT2G45600.1 uORF ′5 STOP: 12 uORF 3′ STOP: 84 leader length: 211 |
| 1662 | AGI number: AT2G45600.1 uORF ′5 STOP: 12 uORF 3′ STOP: 84 leader length: 211 |
| 1663 | AGI number: AT2G45850.1 uORF ′5 STOP: 191 uORF 3′ STOP: 293 leader length: 417 |
| 1664 | AGI number: AT2G45850.1 uORF ′5 STOP: 191 uORF 3′ STOP: 293 leader length: 417 |
| 1665 | AGI number: AT2G45850.1 uORF ′5 STOP: 259 uORF 3′ STOP: 313 leader length: 417 |
| 1666 | AGI number: AT2G45850.1 uORF ′5 STOP: 259 uORF 3′ STOP: 313 leader length: 417 |
| 1667 | AGI number: AT2G46210.1 uORF ′5 STOP: 168 uORF 3′ STOP: 291 leader length: 198 |
| 1668 | AGI number: AT2G46210.1 uORF ′5 STOP: 168 uORF 3′ STOP: 291 leader length: 198 |
| 1669 | AGI number: AT2G46500.2 uORF ′5 STOP: 41 uORF 3′ STOP: 221 leader length: 305 |
| 1670 | AGI number: AT2G46500.2 uORF ′5 STOP: 41 uORF 3′ STOP: 221 leader length: 305 |
| 1671 | AGI number: AT2G46500.2 uORF ′5 STOP: 82 uORF 3′ STOP: 187 leader length: 305 |
| 1672 | AGI number: AT2G46500.2 uORF ′5 STOP: 82 uORF 3′ STOP: 187 leader length: 305 |
| 1673 | AGI number: AT2G46590.1 uORF ′5 STOP: 294 uORF 3′ STOP: 384 leader length: 603 |
| 1674 | AGI number: AT2G46590.1 uORF ′5 STOP: 294 uORF 3′ STOP: 384 leader length: 603 |
| 1675 | AGI number: AT2G46590.1 uORF ′5 STOP: 373 uORF 3′ STOP: 433 leader length: 603 |
| 1676 | AGI number: AT2G46590.1 uORF ′5 STOP: 373 uORF 3′ STOP: 433 leader length: 603 |
| 1677 | AGI number: AT2G46830.1 uORF ′5 STOP: 61 uORF 3′ STOP: 169 leader length: 249 |
| 1678 | AGI number: AT2G46830.1 uORF ′5 STOP: 61 uORF 3′ STOP: 169 leader length: 249 |
| 1679 | AGI number: AT2G47020.2 uORF ′5 STOP: 150 uORF 3′ STOP: 222 leader length: 311 |
| 1680 | AGI number: AT2G47020.2 uORF ′5 STOP: 150 uORF 3′ STOP: 222 leader length: 311 |
| 1681 | AGI number: AT2G47160.1 uORF ′5 STOP: 207 uORF 3′ STOP: 261 leader length: 356 |
| 1682 | AGI number: AT2G47160.1 uORF ′5 STOP: 207 uORF 3′ STOP: 261 leader length: 356 |
| 1683 | AGI number: AT2G47160.1 uORF ′5 STOP: 233 uORF 3′ STOP: 284 leader length: 356 |
| 1684 | AGI number: AT2G47160.1 uORF ′5 STOP: 233 uORF 3′ STOP: 284 leader length: 356 |
| 1685 | AGI number: AT2G47160.2 uORF ′5 STOP: 211 uORF 3′ STOP: 265 leader length: 360 |
| 1686 | AGI number: AT2G47160.2 uORF ′5 STOP: 211 uORF 3′ STOP: 265 leader length: 360 |
| 1687 | AGI number: AT2G47160.2 uORF ′5 STOP: 237 uORF 3′ STOP: 288 leader length: 360 |
| 1688 | AGI number: AT2G47160.2 uORF ′5 STOP: 237 uORF 3′ STOP: 288 leader length: 360 |
| 1689 | AGI number: AT2G47410.2 uORF ′5 STOP: 35 uORF 3′ STOP: 368 leader length: 552 |
| 1690 | AGI number: AT2G47410.2 uORF ′5 STOP: 35 uORF 3′ STOP: 368 leader length: 552 |
| 1691 | AGI number: AT2G47490.1 uORF ′5 STOP: 66 uORF 3′ STOP: 315 leader length: 303 |
| 1692 | AGI number: AT2G47490.1 uORF ′5 STOP: 66 uORF 3′ STOP: 315 leader length: 303 |
| 1693 | AGI number: AT2G47850.2 uORF ′5 STOP: 19 uORF 3′ STOP: 181 leader length: 280 |
| 1694 | AGI number: AT2G47850.2 uORF ′5 STOP: 19 uORF 3′ STOP: 181 leader length: 280 |
| 1695 | AGI number: AT2G47900.1 uORF ′5 STOP: 34 uORF 3′ STOP: 145 leader length: 454 |
| 1696 | AGI number: AT2G47900.1 uORF ′5 STOP: 34 uORF 3′ STOP: 145 leader length: 454 |
| 1697 | AGI number: AT2G47900.3 uORF ′5 STOP: 46 uORF 3′ STOP: 157 leader length: 466 |
| 1698 | AGI number: AT2G47900.3 uORF ′5 STOP: 46 uORF 3′ STOP: 157 leader length: 466 |
| 1699 | AGI number: AT2G47930.1 uORF ′5 STOP: 48 uORF 3′ STOP: 444 leader length: 52 |
| 1700 | AGI number: AT2G47930.1 uORF ′5 STOP: 48 uORF 3′ STOP: 444 leader length: 52 |
| 1701 | AGI number: AT3G01090.1 uORF ′5 STOP: 20 uORF 3′ STOP: 71 leader length: 198 |
| 1702 | AGI number: AT3G01090.1 uORF ′5 STOP: 20 uORF 3′ STOP: 71 leader length: 198 |
| 1703 | AGI number: AT3G01130.1 uORF ′5 STOP: 50 uORF 3′ STOP: 206 leader length: 68 |
| 1704 | AGI number: AT3G01130.1 uORF ′5 STOP: 50 uORF 3′ STOP: 206 leader length: 68 |
| 1705 | AGI number: AT3G01170.1 uORF ′5 STOP: 40 uORF 3′ STOP: 106 leader length: 402 |
| 1706 | AGI number: AT3G01170.1 uORF ′5 STOP: 40 uORF 3′ STOP: 106 leader length: 402 |
| 1707 | AGI number: AT3G01170.1 uORF ′5 STOP: 62 uORF 3′ STOP: 305 leader length: 402 |
| 1708 | AGI number: AT3G01170.1 uORF ′5 STOP: 62 uORF 3′ STOP: 305 leader length: 402 |
| 1709 | AGI number: AT3G01470.1 uORF ′5 STOP: 105 uORF 3′ STOP: 234 leader length: 369 |
| 1710 | AGI number: AT3G01470.1 uORF ′5 STOP: 105 uORF 3′ STOP: 234 leader length: 369 |
| 1711 | AGI number: AT3G01470.1 uORF ′5 STOP: 151 uORF 3′ STOP: 250 leader length: 369 |
| 1712 | AGI number: AT3G01470.1 uORF ′5 STOP: 151 uORF 3′ STOP: 250 leader length: 369 |
| 1713 | AGI number: AT3G01470.1 uORF ′5 STOP: 161 uORF 3′ STOP: 242 leader length: 369 |
| 1714 | AGI number: AT3G01470.1 uORF ′5 STOP: 161 uORF 3′ STOP: 242 leader length: 369 |
| 1715 | AGI number: AT3G01472.1 uORF ′5 STOP: 105 uORF 3′ STOP: 234 leader length: 162 |
| 1716 | AGI number: AT3G01472.1 uORF ′5 STOP: 105 uORF 3′ STOP: 234 leader length: 162 |
| 1717 | AGI number: AT3G01472.1 uORF ′5 STOP: 161 uORF 3′ STOP: 242 leader length: 162 |
| 1718 | AGI number: AT3G01472.1 uORF ′5 STOP: 161 uORF 3′ STOP: 242 leader length: 162 |
| 1719 | AGI number: AT3G01810.3 uORF ′5 STOP: 59 uORF 3′ STOP: 134 leader length: 448 |
| 1720 | AGI number: AT3G01810.3 uORF ′5 STOP: 59 uORF 3′ STOP: 134 leader length: 448 |
| 1721 | AGI number: AT3G01980.1 uORF ′5 STOP: 24 uORF 3′ STOP: 78 leader length: 198 |
| 1722 | AGI number: AT3G01980.1 uORF ′5 STOP: 24 uORF 3′ STOP: 78 leader length: 198 |
| 1723 | AGI number: AT3G02065.1 uORF ′5 STOP: 70 uORF 3′ STOP: 187 leader length: 677 |
| 1724 | AGI number: AT3G02065.1 uORF ′5 STOP: 70 uORF 3′ STOP: 187 leader length: 677 |
| 1725 | AGI number: AT3G02065.1 uORF ′5 STOP: 187 uORF 3′ STOP: 238 leader length: 677 |
| 1726 | AGI number: AT3G02065.1 uORF ′5 STOP: 187 uORF 3′ STOP: 238 leader length: 677 |
| 1727 | AGI number: AT3G02470.1 uORF ′5 STOP: 13 uORF 3′ STOP: 103 leader length: 614 |
| 1728 | AGI number: AT3G02470.1 uORF ′5 STOP: 13 uORF 3′ STOP: 103 leader length: 614 |
| 1729 | AGI number: AT3G02470.1 uORF ′5 STOP: 382 uORF 3′ STOP: 487 leader length: 614 |
| 1730 | AGI number: AT3G02470.1 uORF ′5 STOP: 382 uORF 3′ STOP: 487 leader length: 614 |
| 1731 | AGI number: AT3G02470.1 uORF ′5 STOP: 245 uORF 3′ STOP: 461 leader length: 614 |
| 1732 | AGI number: AT3G02470.1 uORF ′5 STOP: 245 uORF 3′ STOP: 461 leader length: 614 |
| 1733 | AGI number: AT3G02470.1 uORF ′5 STOP: 32 uORF 3′ STOP: 110 leader length: 614 |
| 1734 | AGI number: AT3G02470.1 uORF ′5 STOP: 32 uORF 3′ STOP: 110 leader length: 614 |
| 1735 | AGI number: AT3G02470.1 uORF ′5 STOP: 303 uORF 3′ STOP: 492 leader length: 614 |
| 1736 | AGI number: AT3G02470.1 uORF ′5 STOP: 303 uORF 3′ STOP: 492 leader length: 614 |
| 1737 | AGI number: AT3G02470.3 uORF ′5 STOP: 163 uORF 3′ STOP: 379 leader length: 532 |
| 1738 | AGI number: AT3G02470.3 uORF ′5 STOP: 163 uORF 3′ STOP: 379 leader length: 532 |
| 1739 | AGI number: AT3G02470.3 uORF ′5 STOP: 221 uORF 3′ STOP: 410 leader length: 532 |
| 1740 | AGI number: AT3G02470.3 uORF ′5 STOP: 221 uORF 3′ STOP: 410 leader length: 532 |
| 1741 | AGI number: AT3G02470.3 uORF ′5 STOP: 300 uORF 3′ STOP: 405 leader length: 532 |
| 1742 | AGI number: AT3G02470.3 uORF ′5 STOP: 300 uORF 3′ STOP: 405 leader length: 532 |
| 1743 | AGI number: AT3G02470.4 uORF ′5 STOP: 225 uORF 3′ STOP: 441 leader length: 594 |
| 1744 | AGI number: AT3G02470.4 uORF ′5 STOP: 225 uORF 3′ STOP: 441 leader length: 594 |
| 1745 | AGI number: AT3G02470.4 uORF ′5 STOP: 283 uORF 3′ STOP: 472 leader length: 594 |
| 1746 | AGI number: AT3G02470.4 uORF ′5 STOP: 283 uORF 3′ STOP: 472 leader length: 594 |
| 1747 | AGI number: AT3G02470.4 uORF ′5 STOP: 362 uORF 3′ STOP: 467 leader length: 594 |
| 1748 | AGI number: AT3G02470.4 uORF ′5 STOP: 362 uORF 3′ STOP: 467 leader length: 594 |
| 1749 | AGI number: AT3G02750.1 uORF ′5 STOP: 593 uORF 3′ STOP: 656 leader length: 943 |
| 1750 | AGI number: AT3G02750.1 uORF ′5 STOP: 593 uORF 3′ STOP: 656 leader length: 943 |
| 1751 | AGI number: AT3G02750.1 uORF ′5 STOP: 477 uORF 3′ STOP: 648 leader length: 943 |
| 1752 | AGI number: AT3G02750.1 uORF ′5 STOP: 477 uORF 3′ STOP: 648 leader length: 943 |
| 1753 | AGI number: AT3G02750.1 uORF ′5 STOP: 645 uORF 3′ STOP: 705 leader length: 943 |
| 1754 | AGI number: AT3G02750.1 uORF ′5 STOP: 645 uORF 3′ STOP: 705 leader length: 943 |
| 1755 | AGI number: AT3G02750.1 uORF ′5 STOP: 607 uORF 3′ STOP: 688 leader length: 943 |
| 1756 | AGI number: AT3G02750.1 uORF ′5 STOP: 607 uORF 3′ STOP: 688 leader length: 943 |
| 1757 | AGI number: AT3G03300.3 uORF ′5 STOP: 80 uORF 3′ STOP: 161 leader length: 319 |
| 1758 | AGI number: AT3G03300.3 uORF ′5 STOP: 80 uORF 3′ STOP: 161 leader length: 319 |
| 1759 | AGI number: AT3G03300.3 uORF ′5 STOP: 111 uORF 3′ STOP: 165 leader length: 319 |
| 1760 | AGI number: AT3G03300.3 uORF ′5 STOP: 111 uORF 3′ STOP: 165 leader length: 319 |
| 1761 | AGI number: AT3G03890.1 uORF ′5 STOP: 86 uORF 3′ STOP: 212 leader length: 488 |
| 1762 | AGI number: AT3G03890.1 uORF ′5 STOP: 86 uORF 3′ STOP: 212 leader length: 488 |
| 1763 | AGI number: AT3G03970.3 uORF ′5 STOP: 391 uORF 3′ STOP: 457 leader length: 714 |
| 1764 | AGI number: AT3G03970.3 uORF ′5 STOP: 391 uORF 3′ STOP: 457 leader length: 714 |
| 1765 | AGI number: AT3G04450.2 uORF ′5 STOP: 70 uORF 3′ STOP: 262 leader length: 558 |
| 1766 | AGI number: AT3G04450.2 uORF ′5 STOP: 70 uORF 3′ STOP: 262 leader length: 558 |
| 1767 | AGI number: AT3G04580.1 uORF ′5 STOP: 96 uORF 3′ STOP: 168 leader length: 510 |
| 1768 | AGI number: AT3G04580.1 uORF ′5 STOP: 96 uORF 3′ STOP: 168 leader length: 510 |
| 1769 | AGI number: AT3G04670.1 uORF ′5 STOP: 323 uORF 3′ STOP: 404 leader length: 596 |
| 1770 | AGI number: AT3G04670.1 uORF ′5 STOP: 323 uORF 3′ STOP: 404 leader length: 596 |
| 1771 | AGI number: AT3G04670.2 uORF ′5 STOP: 196 uORF 3′ STOP: 247 leader length: 659 |
| 1772 | AGI number: AT3G04670.2 uORF ′5 STOP: 196 uORF 3′ STOP: 247 leader length: 659 |
| 1773 | AGI number: AT3G04670.2 uORF ′5 STOP: 386 uORF 3′ STOP: 467 leader length: 659 |
| 1774 | AGI number: AT3G04670.2 uORF ′5 STOP: 386 uORF 3′ STOP: 467 leader length: 659 |
| 1775 | AGI number: AT3G04810.1 uORF ′5 STOP: 62 uORF 3′ STOP: 155 leader length: 240 |
| 1776 | AGI number: AT3G04810.1 uORF ′5 STOP: 62 uORF 3′ STOP: 155 leader length: 240 |
| 1777 | AGI number: AT3G04910.1 uORF ′5 STOP: 201 uORF 3′ STOP: 255 leader length: 449 |
| 1778 | AGI number: AT3G04910.1 uORF ′5 STOP: 201 uORF 3′ STOP: 255 leader length: 449 |
| 1779 | AGI number: AT3G04910.2 uORF ′5 STOP: 201 uORF 3′ STOP: 255 leader length: 516 |
| 1780 | AGI number: AT3G04910.2 uORF ′5 STOP: 201 uORF 3′ STOP: 255 leader length: 516 |
| 1781 | AGI number: AT3G04910.3 uORF ′5 STOP: 599 uORF 3′ STOP: 680 leader length: 798 |
| 1782 | AGI number: AT3G04910.3 uORF ′5 STOP: 599 uORF 3′ STOP: 680 leader length: 798 |
| 1783 | AGI number: AT3G04910.3 uORF ′5 STOP: 94 uORF 3′ STOP: 148 leader length: 798 |
| 1784 | AGI number: AT3G04910.3 uORF ′5 STOP: 94 uORF 3′ STOP: 148 leader length: 798 |
| 1785 | AGI number: AT3G04910.3 uORF ′5 STOP: 196 uORF 3′ STOP: 688 leader length: 798 |
| 1786 | AGI number: AT3G04910.3 uORF ′5 STOP: 196 uORF 3′ STOP: 688 leader length: 798 |
| 1787 | AGI number: AT3G04910.3 uORF ′5 STOP: 588 uORF 3′ STOP: 657 leader length: 798 |
| 1788 | AGI number: AT3G04910.3 uORF ′5 STOP: 588 uORF 3′ STOP: 657 leader length: 798 |
| 1789 | AGI number: AT3G05030.1 uORF ′5 STOP: 211 uORF 3′ STOP: 268 leader length: 347 |
| 1790 | AGI number: AT3G05030.1 uORF ′5 STOP: 211 uORF 3′ STOP: 268 leader length: 347 |
| 1791 | AGI number: AT3G05030.1 uORF ′5 STOP: 92 uORF 3′ STOP: 278 leader length: 347 |
| 1792 | AGI number: AT3G05030.1 uORF ′5 STOP: 92 uORF 3′ STOP: 278 leader length: 347 |
| 1793 | AGI number: AT3G05030.1 uORF ′5 STOP: 129 uORF 3′ STOP: 297 leader length: 347 |
| 1794 | AGI number: AT3G05030.1 uORF ′5 STOP: 129 uORF 3′ STOP: 297 leader length: 347 |
| 1795 | AGI number: AT3G05160.2 uORF ′5 STOP: 192 uORF 3′ STOP: 246 leader length: 587 |
| 1796 | AGI number: AT3G05160.2 uORF ′5 STOP: 192 uORF 3′ STOP: 246 leader length: 587 |
| 1797 | AGI number: AT3G05380.4 uORF ′5 STOP: 2 uORF 3′ STOP: 68 leader length: 127 |
| 1798 | AGI number: AT3G05380.4 uORF ′5 STOP: 2 uORF 3′ STOP: 68 leader length: 127 |
| 1799 | AGI number: AT3G05380.4 uORF ′5 STOP: 65 uORF 3′ STOP: 116 leader length: 127 |
| 1800 | AGI number: AT3G05380.4 uORF ′5 STOP: 65 uORF 3′ STOP: 116 leader length: 127 |
| 1801 | AGI number: AT3G05560.3 uORF ′5 STOP: 101 uORF 3′ STOP: 152 leader length: 292 |
| 1802 | AGI number: AT3G05560.3 uORF ′5 STOP: 101 uORF 3′ STOP: 152 leader length: 292 |
| 1803 | AGI number: AT3G05580.1 uORF ′5 STOP: 13 uORF 3′ STOP: 85 leader length: 270 |
| 1804 | AGI number: AT3G05580.1 uORF ′5 STOP: 13 uORF 3′ STOP: 85 leader length: 270 |
| 1805 | AGI number: AT3G05690.1 uORF ′5 STOP: 16 uORF 3′ STOP: 91 leader length: 200 |
| 1806 | AGI number: AT3G05690.1 uORF ′5 STOP: 16 uORF 3′ STOP: 91 leader length: 200 |
| 1807 | AGI number: AT3G05710.1 uORF ′5 STOP: 69 uORF 3′ STOP: 174 leader length: 266 |
| 1808 | AGI number: AT3G05710.1 uORF ′5 STOP: 69 uORF 3′ STOP: 174 leader length: 266 |
| 1809 | AGI number: AT3G05710.2 uORF ′5 STOP: 62 uORF 3′ STOP: 167 leader length: 259 |
| 1810 | AGI number: AT3G05710.2 uORF ′5 STOP: 62 uORF 3′ STOP: 167 leader length: 259 |
| 1811 | AGI number: AT3G05750.1 uORF ′5 STOP: 88 uORF 3′ STOP: 271 leader length: 362 |
| 1812 | AGI number: AT3G05750.1 uORF ′5 STOP: 88 uORF 3′ STOP: 271 leader length: 362 |
| 1813 | AGI number: AT3G05750.1 uORF ′5 STOP: 188 uORF 3′ STOP: 251 leader length: 362 |
| 1814 | AGI number: AT3G05750.1 uORF ′5 STOP: 188 uORF 3′ STOP: 251 leader length: 362 |
| 1815 | AGI number: AT3G05937.1 uORF ′5 STOP: 290 uORF 3′ STOP: 461 leader length: 456 |
| 1816 | AGI number: AT3G05937.1 uORF ′5 STOP: 290 uORF 3′ STOP: 461 leader length: 456 |
| 1817 | AGI number: AT3G06270.1 uORF ′5 STOP: 26 uORF 3′ STOP: 206 leader length: 626 |
| 1818 | AGI number: AT3G06270.1 uORF ′5 STOP: 26 uORF 3′ STOP: 206 leader length: 626 |
| 1819 | AGI number: AT3G06270.1 uORF ′5 STOP: 296 uORF 3′ STOP: 551 leader length: 626 |
| 1820 | AGI number: AT3G06270.1 uORF ′5 STOP: 296 uORF 3′ STOP: 551 leader length: 626 |
| 1821 | AGI number: AT3G06360.1 uORF ′5 STOP: 20 uORF 3′ STOP: 188 leader length: 74 |
| 1822 | AGI number: AT3G06360.1 uORF ′5 STOP: 20 uORF 3′ STOP: 188 leader length: 74 |
| 1823 | AGI number: AT3G06620.1 uORF ′5 STOP: 69 uORF 3′ STOP: 171 leader length: 278 |
| 1824 | AGI number: AT3G06620.1 uORF ′5 STOP: 69 uORF 3′ STOP: 171 leader length: 278 |
| 1825 | AGI number: AT3G07100.1 uORF ′5 STOP: 276 uORF 3′ STOP: 426 leader length: 544 |
| 1826 | AGI number: AT3G07100.1 uORF ′5 STOP: 276 uORF 3′ STOP: 426 leader length: 544 |
| 1827 | AGI number: AT3G07170.1 uORF ′5 STOP: 10 uORF 3′ STOP: 97 leader length: 234 |
| 1828 | AGI number: AT3G07170.1 uORF ′5 STOP: 10 uORF 3′ STOP: 97 leader length: 234 |
| 1829 | AGI number: AT3G07210.1 uORF ′5 STOP: 4 uORF 3′ STOP: 181 leader length: 333 |
| 1830 | AGI number: AT3G07210.1 uORF ′5 STOP: 4 uORF 3′ STOP: 181 leader length: 333 |
| 1831 | AGI number: AT3G07550.1 uORF ′5 STOP: 14 uORF 3′ STOP: 83 leader length: 359 |
| 1832 | AGI number: AT3G07550.1 uORF ′5 STOP: 14 uORF 3′ STOP: 83 leader length: 359 |
| 1833 | AGI number: AT3G08510.2 uORF ′5 STOP: 11 uORF 3′ STOP: 209 leader length: 295 |
| 1834 | AGI number: AT3G08510.2 uORF ′5 STOP: 11 uORF 3′ STOP: 209 leader length: 295 |
| 1835 | AGI number: AT3G08680.2 uORF ′5 STOP: 59 uORF 3′ STOP: 293 leader length: 477 |
| 1836 | AGI number: AT3G08680.2 uORF ′5 STOP: 59 uORF 3′ STOP: 293 leader length: 477 |
| 1837 | AGI number: AT3G08730.1 uORF ′5 STOP: 159 uORF 3′ STOP: 228 leader length: 332 |
| 1838 | AGI number: AT3G08730.1 uORF ′5 STOP: 159 uORF 3′ STOP: 228 leader length: 332 |
| 1839 | AGI number: AT3G08730.1 uORF ′5 STOP: 103 uORF 3′ STOP: 247 leader length: 332 |
| 1840 | AGI number: AT3G08730.1 uORF ′5 STOP: 103 uORF 3′ STOP: 247 leader length: 332 |
| 1841 | AGI number: AT3G08980.1 uORF ′5 STOP: 33 uORF 3′ STOP: 213 leader length: 151 |
| 1842 | AGI number: AT3G08980.1 uORF ′5 STOP: 33 uORF 3′ STOP: 213 leader length: 151 |
| 1843 | AGI number: AT3G08980.1 uORF ′5 STOP: 133 uORF 3′ STOP: 232 leader length: 151 |
| 1844 | AGI number: AT3G08980.1 uORF ′5 STOP: 133 uORF 3′ STOP: 232 leader length: 151 |
| 1845 | AGI number: AT3G09150.3 uORF ′5 STOP: 208 uORF 3′ STOP: 262 leader length: 475 |
| 1846 | AGI number: AT3G09150.3 uORF ′5 STOP: 208 uORF 3′ STOP: 262 leader length: 475 |
| 1847 | AGI number: AT3G09350.2 uORF ′5 STOP: 122 uORF 3′ STOP: 257 leader length: 335 |
| 1848 | AGI number: AT3G09350.2 uORF ′5 STOP: 122 uORF 3′ STOP: 257 leader length: 335 |
| 1849 | AGI number: AT3G09350.3 uORF ′5 STOP: 70 uORF 3′ STOP: 280 leader length: 579 |
| 1850 | AGI number: AT3G09350.3 uORF ′5 STOP: 70 uORF 3′ STOP: 280 leader length: 579 |
| 1851 | AGI number: AT3G09350.3 uORF ′5 STOP: 207 uORF 3′ STOP: 276 leader length: 579 |
| 1852 | AGI number: AT3G09350.3 uORF ′5 STOP: 207 uORF 3′ STOP: 276 leader length: 579 |
| 1853 | AGI number: AT3G09350.3 uORF ′5 STOP: 122 uORF 3′ STOP: 257 leader length: 579 |
| 1854 | AGI number: AT3G09350.3 uORF ′5 STOP: 122 uORF 3′ STOP: 257 leader length: 579 |
| 1855 | AGI number: AT3G09560.1 uORF ′5 STOP: 158 uORF 3′ STOP: 242 leader length: 460 |
| 1856 | AGI number: AT3G09560.1 uORF ′5 STOP: 158 uORF 3′ STOP: 242 leader length: 460 |
| 1857 | AGI number: AT3G09820.2 uORF ′5 STOP: 40 uORF 3′ STOP: 205 leader length: 445 |
| 1858 | AGI number: AT3G09820.2 uORF ′5 STOP: 40 uORF 3′ STOP: 205 leader length: 445 |
| 1859 | AGI number: AT3G09820.2 uORF ′5 STOP: 15 uORF 3′ STOP: 138 leader length: 445 |
| 1860 | AGI number: AT3G09820.2 uORF ′5 STOP: 15 uORF 3′ STOP: 138 leader length: 445 |
| 1861 | AGI number: AT3G09920.1 uORF ′5 STOP: 98 uORF 3′ STOP: 179 leader length: 236 |
| 1862 | AGI number: AT3G09920.1 uORF ′5 STOP: 98 uORF 3′ STOP: 179 leader length: 236 |
| 1863 | AGI number: AT3G09920.2 uORF ′5 STOP: 154 uORF 3′ STOP: 214 leader length: 296 |
| 1864 | AGI number: AT3G09920.2 uORF ′5 STOP: 154 uORF 3′ STOP: 214 leader length: 296 |
| 1865 | AGI number: AT3G09920.2 uORF ′5 STOP: 158 uORF 3′ STOP: 239 leader length: 296 |
| 1866 | AGI number: AT3G09920.2 uORF ′5 STOP: 158 uORF 3′ STOP: 239 leader length: 296 |
| 1867 | AGI number: AT3G09920.3 uORF ′5 STOP: 305 uORF 3′ STOP: 365 leader length: 447 |
| 1868 | AGI number: AT3G09920.3 uORF ′5 STOP: 305 uORF 3′ STOP: 365 leader length: 447 |
| 1869 | AGI number: AT3G10640.2 uORF ′5 STOP: 3 uORF 3′ STOP: 207 leader length: 562 |
| 1870 | AGI number: AT3G10640.2 uORF ′5 STOP: 3 uORF 3′ STOP: 207 leader length: 562 |
| 1871 | AGI number: AT3G10910.1 uORF ′5 STOP: 156 uORF 3′ STOP: 345 leader length: 406 |
| 1872 | AGI number: AT3G10910.1 uORF ′5 STOP: 156 uORF 3′ STOP: 345 leader length: 406 |
| 1873 | AGI number: AT3G10915.4 uORF ′5 STOP: 92 uORF 3′ STOP: 161 leader length: 291 |
| 1874 | AGI number: AT3G10915.4 uORF ′5 STOP: 92 uORF 3′ STOP: 161 leader length: 291 |
| 1875 | AGI number: AT3G10915.4 uORF ′5 STOP: 123 uORF 3′ STOP: 186 leader length: 291 |
| 1876 | AGI number: AT3G10915.4 uORF ′5 STOP: 123 uORF 3′ STOP: 186 leader length: 291 |
| 1877 | AGI number: AT3G10915.5 uORF ′5 STOP: 45 uORF 3′ STOP: 123 leader length: 291 |
| 1878 | AGI number: AT3G10915.5 uORF ′5 STOP: 45 uORF 3′ STOP: 123 leader length: 291 |
| 1879 | AGI number: AT3G10930.1 uORF ′5 STOP: 262 uORF 3′ STOP: 352 leader length: 299 |
| 1880 | AGI number: AT3G10930.1 uORF ′5 STOP: 262 uORF 3′ STOP: 352 leader length: 299 |
| 1881 | AGI number: AT3G10930.1 uORF ′5 STOP: 50 uORF 3′ STOP: 368 leader length: 299 |
| 1882 | AGI number: AT3G10930.1 uORF ′5 STOP: 50 uORF 3′ STOP: 368 leader length: 299 |
| 1883 | AGI number: AT3G11200.2 uORF ′5 STOP: 173 uORF 3′ STOP: 233 leader length: 357 |
| 1884 | AGI number: AT3G11200.2 uORF ′5 STOP: 173 uORF 3′ STOP: 233 leader length: 357 |
| 1885 | AGI number: AT3G11200.2 uORF ′5 STOP: 118 uORF 3′ STOP: 241 leader length: 357 |
| 1886 | AGI number: AT3G11200.2 uORF ′5 STOP: 118 uORF 3′ STOP: 241 leader length: 357 |
| 1887 | AGI number: AT3G11200.2 uORF ′5 STOP: 306 uORF 3′ STOP: 375 leader length: 357 |
| 1888 | AGI number: AT3G11200.2 uORF ′5 STOP: 306 uORF 3′ STOP: 375 leader length: 357 |
| 1889 | AGI number: AT3G11320.1 uORF ′5 STOP: 9 uORF 3′ STOP: 174 leader length: 317 |
| 1890 | AGI number: AT3G11320.1 uORF ′5 STOP: 9 uORF 3′ STOP: 174 leader length: 317 |
| 1891 | AGI number: AT3G11530.2 uORF ′5 STOP: 56 uORF 3′ STOP: 236 leader length: 546 |
| 1892 | AGI number: AT3G11530.2 uORF ′5 STOP: 56 uORF 3′ STOP: 236 leader length: 546 |
| 1893 | AGI number: AT3G11850.2 uORF ′5 STOP: 218 uORF 3′ STOP: 425 leader length: 520 |
| 1894 | AGI number: AT3G11850.2 uORF ′5 STOP: 218 uORF 3′ STOP: 425 leader length: 520 |
| 1895 | AGI number: AT3G11850.2 uORF ′5 STOP: 250 uORF 3′ STOP: 430 leader length: 520 |
| 1896 | AGI number: AT3G11850.2 uORF ′5 STOP: 250 uORF 3′ STOP: 430 leader length: 520 |
| 1897 | AGI number: AT3G12200.2 uORF ′5 STOP: 204 uORF 3′ STOP: 270 leader length: 450 |
| 1898 | AGI number: AT3G12200.2 uORF ′5 STOP: 204 uORF 3′ STOP: 270 leader length: 450 |
| 1899 | AGI number: AT3G12280.2 uORF ′5 STOP: 62 uORF 3′ STOP: 143 leader length: 297 |
| 1900 | AGI number: AT3G12280.2 uORF ′5 STOP: 62 uORF 3′ STOP: 143 leader length: 297 |
| 1901 | AGI number: AT3G12550.1 uORF ′5 STOP: 207 uORF 3′ STOP: 306 leader length: 285 |
| 1902 | AGI number: AT3G12550.1 uORF ′5 STOP: 207 uORF 3′ STOP: 306 leader length: 285 |
| 1903 | AGI number: AT3G12570.2 uORF ′5 STOP: 194 uORF 3′ STOP: 308 leader length: 381 |
| 1904 | AGI number: AT3G12570.2 uORF ′5 STOP: 194 uORF 3′ STOP: 308 leader length: 381 |
| 1905 | AGI number: AT3G12570.2 uORF ′5 STOP: 238 uORF 3′ STOP: 304 leader length: 381 |
| 1906 | AGI number: AT3G12570.2 uORF ′5 STOP: 238 uORF 3′ STOP: 304 leader length: 381 |
| 1907 | AGI number: AT3G12620.2 uORF ′5 STOP: 43 uORF 3′ STOP: 133 leader length: 354 |
| 1908 | AGI number: AT3G12620.2 uORF ′5 STOP: 43 uORF 3′ STOP: 133 leader length: 354 |
| 1909 | AGI number: AT3G12620.2 uORF ′5 STOP: 77 uORF 3′ STOP: 164 leader length: 354 |
| 1910 | AGI number: AT3G12620.2 uORF ′5 STOP: 77 uORF 3′ STOP: 164 leader length: 354 |
| 1911 | AGI number: AT3G12770.1 uORF ′5 STOP: 17 uORF 3′ STOP: 134 leader length: 21 |
| 1912 | AGI number: AT3G12770.1 uORF ′5 STOP: 17 uORF 3′ STOP: 134 leader length: 21 |
| 1913 | AGI number: AT3G13000.1 uORF ′5 STOP: 113 uORF 3′ STOP: 275 leader length: 536 |
| 1914 | AGI number: AT3G13000.1 uORF ′5 STOP: 113 uORF 3′ STOP: 275 leader length: 536 |
| 1915 | AGI number: AT3G13030.3 uORF ′5 STOP: 155 uORF 3′ STOP: 260 leader length: 252 |
| 1916 | AGI number: AT3G13030.3 uORF ′5 STOP: 155 uORF 3′ STOP: 260 leader length: 252 |
| 1917 | AGI number: AT3G13190.1 uORF ′5 STOP: 101 uORF 3′ STOP: 158 leader length: 287 |
| 1918 | AGI number: AT3G13190.1 uORF ′5 STOP: 101 uORF 3′ STOP: 158 leader length: 287 |
| 1919 | AGI number: AT3G13190.2 uORF ′5 STOP: 62 uORF 3′ STOP: 119 leader length: 253 |
| 1920 | AGI number: AT3G13190.2 uORF ′5 STOP: 62 uORF 3′ STOP: 119 leader length: 253 |
| 1921 | AGI number: AT3G13430.2 uORF ′5 STOP: 46 uORF 3′ STOP: 124 leader length: 247 |
| 1922 | AGI number: AT3G13430.2 uORF ′5 STOP: 46 uORF 3′ STOP: 124 leader length: 247 |
| 1923 | AGI number: AT3G13440.1 uORF ′5 STOP: 149 uORF 3′ STOP: 329 leader length: 982 |
| 1924 | AGI number: AT3G13440.1 uORF ′5 STOP: 149 uORF 3′ STOP: 329 leader length: 982 |
| 1925 | AGI number: AT3G13580.3 uORF ′5 STOP: 8 uORF 3′ STOP: 122 leader length: 638 |
| 1926 | AGI number: AT3G13580.3 uORF ′5 STOP: 8 uORF 3′ STOP: 122 leader length: 638 |
| 1927 | AGI number: AT3G13690.1 uORF ′5 STOP: 413 uORF 3′ STOP: 665 leader length: 839 |
| 1928 | AGI number: AT3G13690.1 uORF ′5 STOP: 413 uORF 3′ STOP: 665 leader length: 839 |
| 1929 | AGI number: AT3G13690.1 uORF ′5 STOP: 568 uORF 3′ STOP: 685 leader length: 839 |
| 1930 | AGI number: AT3G13690.1 uORF ′5 STOP: 568 uORF 3′ STOP: 685 leader length: 839 |
| 1931 | AGI number: AT3G13700.1 uORF ′5 STOP: 710 uORF 3′ STOP: 830 leader length: 1105 |
| 1932 | AGI number: AT3G13700.1 uORF ′5 STOP: 710 uORF 3′ STOP: 830 leader length: 1105 |
| 1933 | AGI number: AT3G13700.1 uORF ′5 STOP: 123 uORF 3′ STOP: 747 leader length: 1105 |
| 1934 | AGI number: AT3G13700.1 uORF ′5 STOP: 123 uORF 3′ STOP: 747 leader length: 1105 |
| 1935 | AGI number: AT3G13700.1 uORF ′5 STOP: 661 uORF 3′ STOP: 727 leader length: 1105 |
| 1936 | AGI number: AT3G13700.1 uORF ′5 STOP: 661 uORF 3′ STOP: 727 leader length: 1105 |
| 1937 | AGI number: AT3G13700.2 uORF ′5 STOP: 123 uORF 3′ STOP: 747 leader length: 1105 |
| 1938 | AGI number: AT3G13700.2 uORF ′5 STOP: 123 uORF 3′ STOP: 747 leader length: 1105 |
| 1939 | AGI number: AT3G13700.2 uORF ′5 STOP: 661 uORF 3′ STOP: 727 leader length: 1105 |
| 1940 | AGI number: AT3G13700.2 uORF ′5 STOP: 661 uORF 3′ STOP: 727 leader length: 1105 |
| 1941 | AGI number: AT3G13857.1 uORF ′5 STOP: 18 uORF 3′ STOP: 69 leader length: 64 |
| 1942 | AGI number: AT3G13857.1 uORF ′5 STOP: 18 uORF 3′ STOP: 69 leader length: 64 |
| 1943 | AGI number: AT3G14010.4 uORF ′5 STOP: 94 uORF 3′ STOP: 235 leader length: 379 |
| 1944 | AGI number: AT3G14010.4 uORF ′5 STOP: 94 uORF 3′ STOP: 235 leader length: 379 |
| 1945 | AGI number: AT3G14020.1 uORF ′5 STOP: 28 uORF 3′ STOP: 103 leader length: 314 |
| 1946 | AGI number: AT3G14020.1 uORF ′5 STOP: 28 uORF 3′ STOP: 103 leader length: 314 |
| 1947 | AGI number: AT3G14110.2 uORF ′5 STOP: 239 uORF 3′ STOP: 299 leader length: 439 |
| 1948 | AGI number: AT3G14110.2 uORF ′5 STOP: 239 uORF 3′ STOP: 299 leader length: 439 |
| 1949 | AGI number: AT3G14110.2 uORF ′5 STOP: 178 uORF 3′ STOP: 322 leader length: 439 |
| 1950 | AGI number: AT3G14110.2 uORF ′5 STOP: 178 uORF 3′ STOP: 322 leader length: 439 |
| 1951 | AGI number: AT3G14172.1 uORF ′5 STOP: 78 uORF 3′ STOP: 168 leader length: 395 |
| 1952 | AGI number: AT3G14172.1 uORF ′5 STOP: 78 uORF 3′ STOP: 168 leader length: 395 |
| 1953 | AGI number: AT3G14172.2 uORF ′5 STOP: 98 uORF 3′ STOP: 188 leader length: 415 |
| 1954 | AGI number: AT3G14172.2 uORF ′5 STOP: 98 uORF 3′ STOP: 188 leader length: 415 |
| 1955 | AGI number: AT3G14172.2 uORF ′5 STOP: 198 uORF 3′ STOP: 321 leader length: 415 |
| 1956 | AGI number: AT3G14172.2 uORF ′5 STOP: 198 uORF 3′ STOP: 321 leader length: 415 |
| 1957 | AGI number: AT3G14415.3 uORF ′5 STOP: 6 uORF 3′ STOP: 180 leader length: 295 |
| 1958 | AGI number: AT3G14415.3 uORF ′5 STOP: 6 uORF 3′ STOP: 180 leader length: 295 |
| 1959 | AGI number: AT3G14590.1 uORF ′5 STOP: 31 uORF 3′ STOP: 151 leader length: 487 |
| 1960 | AGI number: AT3G14590.1 uORF ′5 STOP: 31 uORF 3′ STOP: 151 leader length: 487 |
| 1961 | AGI number: AT3G14590.1 uORF ′5 STOP: 101 uORF 3′ STOP: 164 leader length: 487 |
| 1962 | AGI number: AT3G14590.1 uORF ′5 STOP: 101 uORF 3′ STOP: 164 leader length: 487 |
| 1963 | AGI number: AT3G14590.2 uORF ′5 STOP: 30 uORF 3′ STOP: 150 leader length: 251 |
| 1964 | AGI number: AT3G14590.2 uORF ′5 STOP: 30 uORF 3′ STOP: 150 leader length: 251 |
| 1965 | AGI number: AT3G14590.2 uORF ′5 STOP: 100 uORF 3′ STOP: 163 leader length: 251 |
| 1966 | AGI number: AT3G14590.2 uORF ′5 STOP: 100 uORF 3′ STOP: 163 leader length: 251 |
| 1967 | AGI number: AT3G15030.3 uORF ′5 STOP: 92 uORF 3′ STOP: 149 leader length: 307 |
| 1968 | AGI number: AT3G15030.3 uORF ′5 STOP: 92 uORF 3′ STOP: 149 leader length: 307 |
| 1969 | AGI number: AT3G15220.1 uORF ′5 STOP: 2 uORF 3′ STOP: 89 leader length: 199 |
| 1970 | AGI number: AT3G15220.1 uORF ′5 STOP: 2 uORF 3′ STOP: 89 leader length: 199 |
| 1971 | AGI number: AT3G15220.1 uORF ′5 STOP: 9 uORF 3′ STOP: 84 leader length: 199 |
| 1972 | AGI number: AT3G15220.1 uORF ′5 STOP: 9 uORF 3′ STOP: 84 leader length: 199 |
| 1973 | AGI number: AT3G15350.1 uORF ′5 STOP: 70 uORF 3′ STOP: 295 leader length: 420 |
| 1974 | AGI number: AT3G15350.1 uORF ′5 STOP: 70 uORF 3′ STOP: 295 leader length: 420 |
| 1975 | AGI number: AT3G15350.1 uORF ′5 STOP: 105 uORF 3′ STOP: 156 leader length: 420 |
| 1976 | AGI number: AT3G15350.1 uORF ′5 STOP: 105 uORF 3′ STOP: 156 leader length: 420 |
| 1977 | AGI number: AT3G15351.3 uORF ′5 STOP: 52 uORF 3′ STOP: 121 leader length: 213 |
| 1978 | AGI number: AT3G15351.3 uORF ′5 STOP: 52 uORF 3′ STOP: 121 leader length: 213 |
| 1979 | AGI number: AT3G15351.3 uORF ′5 STOP: 69 uORF 3′ STOP: 147 leader length: 213 |
| 1980 | AGI number: AT3G15351.3 uORF ′5 STOP: 69 uORF 3′ STOP: 147 leader length: 213 |
| 1981 | AGI number: AT3G15354.1 uORF ′5 STOP: 108 uORF 3′ STOP: 171 leader length: 357 |
| 1982 | AGI number: AT3G15354.1 uORF ′5 STOP: 108 uORF 3′ STOP: 171 leader length: 357 |
| 1983 | AGI number: AT3G15430.1 uORF ′5 STOP: 199 uORF 3′ STOP: 313 leader length: 388 |
| 1984 | AGI number: AT3G15430.1 uORF ′5 STOP: 199 uORF 3′ STOP: 313 leader length: 388 |
| 1985 | AGI number: AT3G15430.1 uORF ′5 STOP: 173 uORF 3′ STOP: 305 leader length: 388 |
| 1986 | AGI number: AT3G15430.1 uORF ′5 STOP: 173 uORF 3′ STOP: 305 leader length: 388 |
| 1987 | AGI number: AT3G15430.2 uORF ′5 STOP: 185 uORF 3′ STOP: 425 leader length: 516 |
| 1988 | AGI number: AT3G15430.2 uORF ′5 STOP: 185 uORF 3′ STOP: 425 leader length: 516 |
| 1989 | AGI number: AT3G15430.2 uORF ′5 STOP: 301 uORF 3′ STOP: 433 leader length: 516 |
| 1990 | AGI number: AT3G15430.2 uORF ′5 STOP: 301 uORF 3′ STOP: 433 leader length: 516 |
| 1991 | AGI number: AT3G15430.2 uORF ′5 STOP: 327 uORF 3′ STOP: 441 leader length: 516 |
| 1992 | AGI number: AT3G15430.2 uORF ′5 STOP: 327 uORF 3′ STOP: 441 leader length: 516 |
| 1993 | AGI number: AT3G17120.1 uORF ′5 STOP: 115 uORF 3′ STOP: 259 leader length: 762 |
| 1994 | AGI number: AT3G17120.1 uORF ′5 STOP: 115 uORF 3′ STOP: 259 leader length: 762 |
| 1995 | AGI number: AT3G17120.1 uORF ′5 STOP: 56 uORF 3′ STOP: 113 leader length: 762 |
| 1996 | AGI number: AT3G17120.1 uORF ′5 STOP: 56 uORF 3′ STOP: 113 leader length: 762 |
| 1997 | AGI number: AT3G17310.1 uORF ′5 STOP: 150 uORF 3′ STOP: 300 leader length: 325 |
| 1998 | AGI number: AT3G17310.1 uORF ′5 STOP: 150 uORF 3′ STOP: 300 leader length: 325 |
| 1999 | AGI number: AT3G17450.1 uORF ′5 STOP: 97 uORF 3′ STOP: 148 leader length: 302 |
| 2000 | AGI number: AT3G17450.1 uORF ′5 STOP: 97 uORF 3′ STOP: 148 leader length: 302 |
| 2001 | AGI number: AT3G17580.1 uORF ′5 STOP: 79 uORF 3′ STOP: 373 leader length: 92 |
| 2002 | AGI number: AT3G17580.1 uORF ′5 STOP: 79 uORF 3′ STOP: 373 leader length: 92 |
| 2003 | AGI number: AT3G17609.3 uORF ′5 STOP: 8 uORF 3′ STOP: 107 leader length: 198 |
| 2004 | AGI number: AT3G17609.3 uORF ′5 STOP: 8 uORF 3′ STOP: 107 leader length: 198 |
| 2005 | AGI number: AT3G17611.2 uORF ′5 STOP: 114 uORF 3′ STOP: 219 leader length: 541 |
| 2006 | AGI number: AT3G17611.2 uORF ′5 STOP: 114 uORF 3′ STOP: 219 leader length: 541 |
| 2007 | AGI number: AT3G17611.2 uORF ′5 STOP: 137 uORF 3′ STOP: 254 leader length: 541 |
| 2008 | AGI number: AT3G17611.2 uORF ′5 STOP: 137 uORF 3′ STOP: 254 leader length: 541 |
| 2009 | AGI number: AT3G17950.2 uORF ′5 STOP: 13 uORF 3′ STOP: 133 leader length: 410 |
| 2010 | AGI number: AT3G17950.2 uORF ′5 STOP: 13 uORF 3′ STOP: 133 leader length: 410 |
| 2011 | AGI number: AT3G18160.2 uORF ′5 STOP: 342 uORF 3′ STOP: 474 leader length: 545 |
| 2012 | AGI number: AT3G18160.2 uORF ′5 STOP: 342 uORF 3′ STOP: 474 leader length: 545 |
| 2013 | AGI number: AT3G18370.1 uORF ′5 STOP: 24 uORF 3′ STOP: 81 leader length: 387 |
| 2014 | AGI number: AT3G18370.1 uORF ′5 STOP: 24 uORF 3′ STOP: 81 leader length: 387 |
| 2015 | AGI number: AT3G18620.1 uORF ′5 STOP: 290 uORF 3′ STOP: 344 leader length: 531 |
| 2016 | AGI number: AT3G18620.1 uORF ′5 STOP: 290 uORF 3′ STOP: 344 leader length: 531 |
| 2017 | AGI number: AT3G18770.1 uORF ′5 STOP: 25 uORF 3′ STOP: 127 leader length: 323 |
| 2018 | AGI number: AT3G18770.1 uORF ′5 STOP: 25 uORF 3′ STOP: 127 leader length: 323 |
| 2019 | AGI number: AT3G19250.1 uORF ′5 STOP: 197 uORF 3′ STOP: 251 leader length: 387 |
| 2020 | AGI number: AT3G19250.1 uORF ′5 STOP: 197 uORF 3′ STOP: 251 leader length: 387 |
| 2021 | AGI number: AT3G19250.1 uORF ′5 STOP: 178 uORF 3′ STOP: 259 leader length: 387 |
| 2022 | AGI number: AT3G19250.1 uORF ′5 STOP: 178 uORF 3′ STOP: 259 leader length: 387 |
| 2023 | AGI number: AT3G19440.1 uORF ′5 STOP: 73 uORF 3′ STOP: 253 leader length: 83 |
| 2024 | AGI number: AT3G19440.1 uORF ′5 STOP: 73 uORF 3′ STOP: 253 leader length: 83 |
| 2025 | AGI number: AT3G20040.1 uORF ′5 STOP: 217 uORF 3′ STOP: 277 leader length: 423 |
| 2026 | AGI number: AT3G20040.1 uORF ′5 STOP: 217 uORF 3′ STOP: 277 leader length: 423 |
| 2027 | AGI number: AT3G20040.1 uORF ′5 STOP: 252 uORF 3′ STOP: 315 leader length: 423 |
| 2028 | AGI number: AT3G20040.1 uORF ′5 STOP: 252 uORF 3′ STOP: 315 leader length: 423 |
| 2029 | AGI number: AT3G20200.1 uORF ′5 STOP: 183 uORF 3′ STOP: 294 leader length: 314 |
| 2030 | AGI number: AT3G20200.1 uORF ′5 STOP: 183 uORF 3′ STOP: 294 leader length: 314 |
| 2031 | AGI number: AT3G20260.1 uORF ′5 STOP: 256 uORF 3′ STOP: 349 leader length: 752 |
| 2032 | AGI number: AT3G20260.1 uORF ′5 STOP: 256 uORF 3′ STOP: 349 leader length: 752 |
| 2033 | AGI number: AT3G20260.1 uORF ′5 STOP: 203 uORF 3′ STOP: 356 leader length: 752 |
| 2034 | AGI number: AT3G20260.1 uORF ′5 STOP: 203 uORF 3′ STOP: 356 leader length: 752 |
| 2035 | AGI number: AT3G20430.1 uORF ′5 STOP: 21 uORF 3′ STOP: 90 leader length: 144 |
| 2036 | AGI number: AT3G20430.1 uORF ′5 STOP: 21 uORF 3′ STOP: 90 leader length: 144 |
| 2037 | AGI number: AT3G20620.1 uORF ′5 STOP: 114 uORF 3′ STOP: 255 leader length: 321 |
| 2038 | AGI number: AT3G20620.1 uORF ′5 STOP: 114 uORF 3′ STOP: 255 leader length: 321 |
| 2039 | AGI number: AT3G20810.1 uORF ′5 STOP: 38 uORF 3′ STOP: 251 leader length: 108 |
| 2040 | AGI number: AT3G20810.1 uORF ′5 STOP: 38 uORF 3′ STOP: 251 leader length: 108 |
| 2041 | AGI number: AT3G20870.1 uORF ′5 STOP: 69 uORF 3′ STOP: 123 leader length: 276 |
| 2042 | AGI number: AT3G20870.1 uORF ′5 STOP: 69 uORF 3′ STOP: 123 leader length: 276 |
| 2043 | AGI number: AT3G21250.2 uORF ′5 STOP: 64 uORF 3′ STOP: 166 leader length: 264 |
| 2044 | AGI number: AT3G21250.2 uORF ′5 STOP: 64 uORF 3′ STOP: 166 leader length: 264 |
| 2045 | AGI number: AT3G21250.2 uORF ′5 STOP: 102 uORF 3′ STOP: 171 leader length: 264 |
| 2046 | AGI number: AT3G21250.2 uORF ′5 STOP: 102 uORF 3′ STOP: 171 leader length: 264 |
| 2047 | AGI number: AT3G21295.1 uORF ′5 STOP: 79 uORF 3′ STOP: 136 leader length: 399 |
| 2048 | AGI number: AT3G21295.1 uORF ′5 STOP: 79 uORF 3′ STOP: 136 leader length: 399 |
| 2049 | AGI number: AT3G21310.1 uORF ′5 STOP: 102 uORF 3′ STOP: 156 leader length: 291 |
| 2050 | AGI number: AT3G21310.1 uORF ′5 STOP: 102 uORF 3′ STOP: 156 leader length: 291 |
| 2051 | AGI number: AT3G21700.1 uORF ′5 STOP: 41 uORF 3′ STOP: 119 leader length: 211 |
| 2052 | AGI number: AT3G21700.1 uORF ′5 STOP: 41 uORF 3′ STOP: 119 leader length: 211 |
| 2053 | AGI number: AT3G22415.1 uORF ′5 STOP: 25 uORF 3′ STOP: 220 leader length: 154 |
| 2054 | AGI number: AT3G22415.1 uORF ′5 STOP: 25 uORF 3′ STOP: 220 leader length: 154 |
| 2055 | AGI number: AT3G22550.1 uORF ′5 STOP: 29 uORF 3′ STOP: 149 leader length: 498 |
| 2056 | AGI number: AT3G22550.1 uORF ′5 STOP: 29 uORF 3′ STOP: 149 leader length: 498 |
| 2057 | AGI number: AT3G22550.1 uORF ′5 STOP: 206 uORF 3′ STOP: 284 leader length: 498 |
| 2058 | AGI number: AT3G22550.1 uORF ′5 STOP: 206 uORF 3′ STOP: 284 leader length: 498 |
| 2059 | AGI number: AT3G22750.1 uORF ′5 STOP: 261 uORF 3′ STOP: 369 leader length: 807 |
| 2060 | AGI number: AT3G22750.1 uORF ′5 STOP: 261 uORF 3′ STOP: 369 leader length: 807 |
| 2061 | AGI number: AT3G22750.1 uORF ′5 STOP: 504 uORF 3′ STOP: 561 leader length: 807 |
| 2062 | AGI number: AT3G22750.1 uORF ′5 STOP: 504 uORF 3′ STOP: 561 leader length: 807 |
| 2063 | AGI number: AT3G22750.1 uORF ′5 STOP: 634 uORF 3′ STOP: 721 leader length: 807 |
| 2064 | AGI number: AT3G22750.1 uORF ′5 STOP: 634 uORF 3′ STOP: 721 leader length: 807 |
| 2065 | AGI number: AT3G22970.1 uORF ′5 STOP: 202 uORF 3′ STOP: 310 leader length: 554 |
| 2066 | AGI number: AT3G22970.1 uORF ′5 STOP: 202 uORF 3′ STOP: 310 leader length: 554 |
| 2067 | AGI number: AT3G22970.2 uORF ′5 STOP: 101 uORF 3′ STOP: 221 leader length: 885 |
| 2068 | AGI number: AT3G22970.2 uORF ′5 STOP: 101 uORF 3′ STOP: 221 leader length: 885 |
| 2069 | AGI number: AT3G22970.2 uORF ′5 STOP: 202 uORF 3′ STOP: 310 leader length: 885 |
| 2070 | AGI number: AT3G22970.2 uORF ′5 STOP: 202 uORF 3′ STOP: 310 leader length: 885 |
| 2071 | AGI number: AT3G23160.1 uORF ′5 STOP: 92 uORF 3′ STOP: 230 leader length: 374 |
| 2072 | AGI number: AT3G23160.1 uORF ′5 STOP: 92 uORF 3′ STOP: 230 leader length: 374 |
| 2073 | AGI number: AT3G23160.1 uORF ′5 STOP: 217 uORF 3′ STOP: 283 leader length: 374 |
| 2074 | AGI number: AT3G23160.1 uORF ′5 STOP: 217 uORF 3′ STOP: 283 leader length: 374 |
| 2075 | AGI number: AT3G24070.1 uORF ′5 STOP: 437 uORF 3′ STOP: 503 leader length: 458 |
| 2076 | AGI number: AT3G24070.1 uORF ′5 STOP: 437 uORF 3′ STOP: 503 leader length: 458 |
| 2077 | AGI number: AT3G24070.1 uORF ′5 STOP: 70 uORF 3′ STOP: 448 leader length: 458 |
| 2078 | AGI number: AT3G24070.1 uORF ′5 STOP: 70 uORF 3′ STOP: 448 leader length: 458 |
| 2079 | AGI number: AT3G24490.1 uORF ′5 STOP: 50 uORF 3′ STOP: 134 leader length: 252 |
| 2080 | AGI number: AT3G24490.1 uORF ′5 STOP: 50 uORF 3′ STOP: 134 leader length: 252 |
| 2081 | AGI number: AT3G25570.1 uORF ′5 STOP: 198 uORF 3′ STOP: 369 leader length: 500 |
| 2082 | AGI number: AT3G25570.1 uORF ′5 STOP: 198 uORF 3′ STOP: 369 leader length: 500 |
| 2083 | AGI number: AT3G25570.2 uORF ′5 STOP: 258 uORF 3′ STOP: 354 leader length: 459 |
| 2084 | AGI number: AT3G25570.2 uORF ′5 STOP: 258 uORF 3′ STOP: 354 leader length: 459 |
| 2085 | AGI number: AT3G25585.4 uORF ′5 STOP: 75 uORF 3′ STOP: 129 leader length: 360 |
| 2086 | AGI number: AT3G25585.4 uORF ′5 STOP: 75 uORF 3′ STOP: 129 leader length: 360 |
| 2087 | AGI number: AT3G25585.4 uORF ′5 STOP: 85 uORF 3′ STOP: 175 leader length: 360 |
| 2088 | AGI number: AT3G25585.4 uORF ′5 STOP: 85 uORF 3′ STOP: 175 leader length: 360 |
| 2089 | AGI number: AT3G25590.1 uORF ′5 STOP: 38 uORF 3′ STOP: 119 leader length: 261 |
| 2090 | AGI number: AT3G25590.1 uORF ′5 STOP: 38 uORF 3′ STOP: 119 leader length: 261 |
| 2091 | AGI number: AT3G25597.1 uORF ′5 STOP: 99 uORF 3′ STOP: 333 leader length: 181 |
| 2092 | AGI number: AT3G25597.1 uORF ′5 STOP: 99 uORF 3′ STOP: 333 leader length: 181 |
| 2093 | AGI number: AT3G25840.2 uORF ′5 STOP: 82 uORF 3′ STOP: 475 leader length: 950 |
| 2094 | AGI number: AT3G25840.2 uORF ′5 STOP: 82 uORF 3′ STOP: 475 leader length: 950 |
| 2095 | AGI number: AT3G25840.2 uORF ′5 STOP: 396 uORF 3′ STOP: 453 leader length: 950 |
| 2096 | AGI number: AT3G25840.2 uORF ′5 STOP: 396 uORF 3′ STOP: 453 leader length: 950 |
| 2097 | AGI number: AT3G25840.2 uORF ′5 STOP: 407 uORF 3′ STOP: 467 leader length: 950 |
| 2098 | AGI number: AT3G25840.2 uORF ′5 STOP: 407 uORF 3′ STOP: 467 leader length: 950 |
| 2099 | AGI number: AT3G26000.1 uORF ′5 STOP: 98 uORF 3′ STOP: 296 leader length: 716 |
| 2100 | AGI number: AT3G26000.1 uORF ′5 STOP: 98 uORF 3′ STOP: 296 leader length: 716 |
| 2101 | AGI number: AT3G26000.1 uORF ′5 STOP: 229 uORF 3′ STOP: 289 leader length: 716 |
| 2102 | AGI number: AT3G26000.1 uORF ′5 STOP: 229 uORF 3′ STOP: 289 leader length: 716 |
| 2103 | AGI number: AT3G26085.2 uORF ′5 STOP: 95 uORF 3′ STOP: 269 leader length: 182 |
| 2104 | AGI number: AT3G26085.2 uORF ′5 STOP: 95 uORF 3′ STOP: 269 leader length: 182 |
| 2105 | AGI number: AT3G26085.2 uORF ′5 STOP: 139 uORF 3′ STOP: 298 leader length: 182 |
| 2106 | AGI number: AT3G26085.2 uORF ′5 STOP: 139 uORF 3′ STOP: 298 leader length: 182 |
| 2107 | AGI number: AT3G26100.1 uORF ′5 STOP: 263 uORF 3′ STOP: 326 leader length: 829 |
| 2108 | AGI number: AT3G26100.1 uORF ′5 STOP: 263 uORF 3′ STOP: 326 leader length: 829 |
| 2109 | AGI number: AT3G26100.2 uORF ′5 STOP: 377 uORF 3′ STOP: 440 leader length: 548 |
| 2110 | AGI number: AT3G26100.2 uORF ′5 STOP: 377 uORF 3′ STOP: 440 leader length: 548 |
| 2111 | AGI number: AT3G26230.1 uORF ′5 STOP: 52 uORF 3′ STOP: 307 leader length: 67 |
| 2112 | AGI number: AT3G26230.1 uORF ′5 STOP: 52 uORF 3′ STOP: 307 leader length: 67 |
| 2113 | AGI number: AT3G26430.1 uORF ′5 STOP: 48 uORF 3′ STOP: 828 leader length: 1981 |
| 2114 | AGI number: AT3G26430.1 uORF ′5 STOP: 48 uORF 3′ STOP: 828 leader length: 1981 |
| 2115 | AGI number: AT3G26430.1 uORF ′5 STOP: 797 uORF 3′ STOP: 848 leader length: 1981 |
| 2116 | AGI number: AT3G26430.1 uORF ′5 STOP: 797 uORF 3′ STOP: 848 leader length: 1981 |
| 2117 | AGI number: AT3G26440.4 uORF ′5 STOP: 98 uORF 3′ STOP: 152 leader length: 540 |
| 2118 | AGI number: AT3G26440.4 uORF ′5 STOP: 98 uORF 3′ STOP: 152 leader length: 540 |
| 2119 | AGI number: AT3G26690.2 uORF ′5 STOP: 197 uORF 3′ STOP: 248 leader length: 317 |
| 2120 | AGI number: AT3G26690.2 uORF ′5 STOP: 197 uORF 3′ STOP: 248 leader length: 317 |
| 2121 | AGI number: AT3G26744.1 uORF ′5 STOP: 40 uORF 3′ STOP: 127 leader length: 297 |
| 2122 | AGI number: AT3G26744.1 uORF ′5 STOP: 40 uORF 3′ STOP: 127 leader length: 297 |
| 2123 | AGI number: AT3G26744.2 uORF ′5 STOP: 14 uORF 3′ STOP: 77 leader length: 210 |
| 2124 | AGI number: AT3G26744.2 uORF ′5 STOP: 14 uORF 3′ STOP: 77 leader length: 210 |
| 2125 | AGI number: AT3G26890.1 uORF ′5 STOP: 196 uORF 3′ STOP: 391 leader length: 551 |
| 2126 | AGI number: AT3G26890.1 uORF ′5 STOP: 196 uORF 3′ STOP: 391 leader length: 551 |
| 2127 | AGI number: AT3G26890.1 uORF ′5 STOP: 308 uORF 3′ STOP: 380 leader length: 551 |
| 2128 | AGI number: AT3G26890.1 uORF ′5 STOP: 308 uORF 3′ STOP: 380 leader length: 551 |
| 2129 | AGI number: AT3G26890.1 uORF ′5 STOP: 377 uORF 3′ STOP: 470 leader length: 551 |
| 2130 | AGI number: AT3G26890.1 uORF ′5 STOP: 377 uORF 3′ STOP: 470 leader length: 551 |
| 2131 | AGI number: AT3G26890.2 uORF ′5 STOP: 52 uORF 3′ STOP: 376 leader length: 536 |
| 2132 | AGI number: AT3G26890.2 uORF ′5 STOP: 52 uORF 3′ STOP: 376 leader length: 536 |
| 2133 | AGI number: AT3G26890.2 uORF ′5 STOP: 293 uORF 3′ STOP: 365 leader length: 536 |
| 2134 | AGI number: AT3G26890.2 uORF ′5 STOP: 293 uORF 3′ STOP: 365 leader length: 536 |
| 2135 | AGI number: AT3G26890.3 uORF ′5 STOP: 236 uORF 3′ STOP: 443 leader length: 603 |
| 2136 | AGI number: AT3G26890.3 uORF ′5 STOP: 236 uORF 3′ STOP: 443 leader length: 603 |
| 2137 | AGI number: AT3G26890.3 uORF ′5 STOP: 360 uORF 3′ STOP: 432 leader length: 603 |
| 2138 | AGI number: AT3G26890.3 uORF ′5 STOP: 360 uORF 3′ STOP: 432 leader length: 603 |
| 2139 | AGI number: AT3G26890.4 uORF ′5 STOP: 123 uORF 3′ STOP: 195 leader length: 366 |
| 2140 | AGI number: AT3G26890.4 uORF ′5 STOP: 123 uORF 3′ STOP: 195 leader length: 366 |
| 2141 | AGI number: AT3G26890.5 uORF ′5 STOP: 101 uORF 3′ STOP: 173 leader length: 344 |
| 2142 | AGI number: AT3G26890.5 uORF ′5 STOP: 101 uORF 3′ STOP: 173 leader length: 344 |
| 2143 | AGI number: AT3G26890.6 uORF ′5 STOP: 551 uORF 3′ STOP: 623 leader length: 794 |
| 2144 | AGI number: AT3G26890.6 uORF ′5 STOP: 551 uORF 3′ STOP: 623 leader length: 794 |
| 2145 | AGI number: AT3G26910.1 uORF ′5 STOP: 90 uORF 3′ STOP: 150 leader length: 273 |
| 2146 | AGI number: AT3G26910.1 uORF ′5 STOP: 90 uORF 3′ STOP: 150 leader length: 273 |
| 2147 | AGI number: AT3G26920.1 uORF ′5 STOP: 137 uORF 3′ STOP: 206 leader length: 568 |
| 2148 | AGI number: AT3G26920.1 uORF ′5 STOP: 137 uORF 3′ STOP: 206 leader length: 568 |
| 2149 | AGI number: AT3G27260.2 uORF ′5 STOP: 48 uORF 3′ STOP: 291 leader length: 601 |
| 2150 | AGI number: AT3G27260.2 uORF ′5 STOP: 48 uORF 3′ STOP: 291 leader length: 601 |
| 2151 | AGI number: AT3G27260.2 uORF ′5 STOP: 191 uORF 3′ STOP: 302 leader length: 601 |
| 2152 | AGI number: AT3G27260.2 uORF ′5 STOP: 191 uORF 3′ STOP: 302 leader length: 601 |
| 2153 | AGI number: AT3G27330.1 uORF ′5 STOP: 75 uORF 3′ STOP: 144 leader length: 129 |
| 2154 | AGI number: AT3G27330.1 uORF ′5 STOP: 75 uORF 3′ STOP: 144 leader length: 129 |
| 2155 | AGI number: AT3G27350.3 uORF ′5 STOP: 370 uORF 3′ STOP: 505 leader length: 700 |
| 2156 | AGI number: AT3G27350.3 uORF ′5 STOP: 370 uORF 3′ STOP: 505 leader length: 700 |
| 2157 | AGI number: AT3G27350.3 uORF ′5 STOP: 447 uORF 3′ STOP: 510 leader length: 700 |
| 2158 | AGI number: AT3G27350.3 uORF ′5 STOP: 447 uORF 3′ STOP: 510 leader length: 700 |
| 2159 | AGI number: AT3G27510.1 uORF ′5 STOP: 1 uORF 3′ STOP: 811 leader length: 781 |
| 2160 | AGI number: AT3G27510.1 uORF ′5 STOP: 1 uORF 3′ STOP: 811 leader length: 781 |
| 2161 | AGI number: AT3G27560.1 uORF ′5 STOP: 70 uORF 3′ STOP: 229 leader length: 497 |
| 2162 | AGI number: AT3G27560.1 uORF ′5 STOP: 70 uORF 3′ STOP: 229 leader length: 497 |
| 2163 | AGI number: AT3G27560.1 uORF ′5 STOP: 89 uORF 3′ STOP: 233 leader length: 497 |
| 2164 | AGI number: AT3G27560.1 uORF ′5 STOP: 89 uORF 3′ STOP: 233 leader length: 497 |
| 2165 | AGI number: AT3G27940.1 uORF ′5 STOP: 111 uORF 3′ STOP: 180 leader length: 175 |
| 2166 | AGI number: AT3G27940.1 uORF ′5 STOP: 111 uORF 3′ STOP: 180 leader length: 175 |
| 2167 | AGI number: AT3G28690.1 uORF ′5 STOP: 447 uORF 3′ STOP: 564 leader length: 963 |
| 2168 | AGI number: AT3G28690.1 uORF ′5 STOP: 447 uORF 3′ STOP: 564 leader length: 963 |
| 2169 | AGI number: AT3G28690.1 uORF ′5 STOP: 120 uORF 3′ STOP: 267 leader length: 963 |
| 2170 | AGI number: AT3G28690.1 uORF ′5 STOP: 120 uORF 3′ STOP: 267 leader length: 963 |
| 2171 | AGI number: AT3G28920.1 uORF ′5 STOP: 64 uORF 3′ STOP: 178 leader length: 297 |
| 2172 | AGI number: AT3G28920.1 uORF ′5 STOP: 64 uORF 3′ STOP: 178 leader length: 297 |
| 2173 | AGI number: AT3G29130.2 uORF ′5 STOP: 50 uORF 3′ STOP: 107 leader length: 59 |
| 2174 | AGI number: AT3G29130.2 uORF ′5 STOP: 50 uORF 3′ STOP: 107 leader length: 59 |
| 2175 | AGI number: AT3G41762.1 uORF ′5 STOP: 18 uORF 3′ STOP: 132 leader length: 139 |
| 2176 | AGI number: AT3G41762.1 uORF ′5 STOP: 18 uORF 3′ STOP: 132 leader length: 139 |
| 2177 | AGI number: AT3G42150.1 uORF ′5 STOP: 237 uORF 3′ STOP: 531 leader length: 249 |
| 2178 | AGI number: AT3G42150.1 uORF ′5 STOP: 237 uORF 3′ STOP: 531 leader length: 249 |
| 2179 | AGI number: AT3G42150.2 uORF ′5 STOP: 34 uORF 3′ STOP: 328 leader length: 46 |
| 2180 | AGI number: AT3G42150.2 uORF ′5 STOP: 34 uORF 3′ STOP: 328 leader length: 46 |
| 2181 | AGI number: AT3G42150.3 uORF ′5 STOP: 22 uORF 3′ STOP: 316 leader length: 34 |
| 2182 | AGI number: AT3G42150.3 uORF ′5 STOP: 22 uORF 3′ STOP: 316 leader length: 34 |
| 2183 | AGI number: AT3G43240.1 uORF ′5 STOP: 54 uORF 3′ STOP: 108 leader length: 302 |
| 2184 | AGI number: AT3G43240.1 uORF ′5 STOP: 54 uORF 3′ STOP: 108 leader length: 302 |
| 2185 | AGI number: AT3G44740.1 uORF ′5 STOP: 106 uORF 3′ STOP: 202 leader length: 118 |
| 2186 | AGI number: AT3G44740.1 uORF ′5 STOP: 106 uORF 3′ STOP: 202 leader length: 118 |
| 2187 | AGI number: AT3G45090.1 uORF ′5 STOP: 35 uORF 3′ STOP: 158 leader length: 229 |
| 2188 | AGI number: AT3G45090.1 uORF ′5 STOP: 35 uORF 3′ STOP: 158 leader length: 229 |
| 2189 | AGI number: AT3G45090.2 uORF ′5 STOP: 83 uORF 3′ STOP: 164 leader length: 280 |
| 2190 | AGI number: AT3G45090.2 uORF ′5 STOP: 83 uORF 3′ STOP: 164 leader length: 280 |
| 2191 | AGI number: AT3G45090.2 uORF ′5 STOP: 34 uORF 3′ STOP: 157 leader length: 280 |
| 2192 | AGI number: AT3G45090.2 uORF ′5 STOP: 34 uORF 3′ STOP: 157 leader length: 280 |
| 2193 | AGI number: AT3G45240.1 uORF ′5 STOP: 148 uORF 3′ STOP: 220 leader length: 748 |
| 2194 | AGI number: AT3G45240.1 uORF ′5 STOP: 148 uORF 3′ STOP: 220 leader length: 748 |
| 2195 | AGI number: AT3G45240.1 uORF ′5 STOP: 260 uORF 3′ STOP: 395 leader length: 748 |
| 2196 | AGI number: AT3G45240.1 uORF ′5 STOP: 260 uORF 3′ STOP: 395 leader length: 748 |
| 2197 | AGI number: AT3G45240.1 uORF ′5 STOP: 319 uORF 3′ STOP: 379 leader length: 748 |
| 2198 | AGI number: AT3G45240.1 uORF ′5 STOP: 319 uORF 3′ STOP: 379 leader length: 748 |
| 2199 | AGI number: AT3G45240.1 uORF ′5 STOP: 437 uORF 3′ STOP: 653 leader length: 748 |
| 2200 | AGI number: AT3G45240.1 uORF ′5 STOP: 437 uORF 3′ STOP: 653 leader length: 748 |
| 2201 | AGI number: AT3G45240.2 uORF ′5 STOP: 147 uORF 3′ STOP: 363 leader length: 458 |
| 2202 | AGI number: AT3G45240.2 uORF ′5 STOP: 147 uORF 3′ STOP: 363 leader length: 458 |
| 2203 | AGI number: AT3G45240.3 uORF ′5 STOP: 102 uORF 3′ STOP: 318 leader length: 413 |
| 2204 | AGI number: AT3G45240.3 uORF ′5 STOP: 102 uORF 3′ STOP: 318 leader length: 413 |
| 2205 | AGI number: AT3G45870.2 uORF ′5 STOP: 91 uORF 3′ STOP: 160 leader length: 354 |
| 2206 | AGI number: AT3G45870.2 uORF ′5 STOP: 91 uORF 3′ STOP: 160 leader length: 354 |
| 2207 | AGI number: AT3G45960.1 uORF ′5 STOP: 96 uORF 3′ STOP: 216 leader length: 302 |
| 2208 | AGI number: AT3G45960.1 uORF ′5 STOP: 96 uORF 3′ STOP: 216 leader length: 302 |
| 2209 | AGI number: AT3G45960.1 uORF ′5 STOP: 227 uORF 3′ STOP: 419 leader length: 302 |
| 2210 | AGI number: AT3G45960.1 uORF ′5 STOP: 227 uORF 3′ STOP: 419 leader length: 302 |
| 2211 | AGI number: AT3G46590.1 uORF ′5 STOP: 120 uORF 3′ STOP: 171 leader length: 376 |
| 2212 | AGI number: AT3G46590.1 uORF ′5 STOP: 120 uORF 3′ STOP: 171 leader length: 376 |
| 2213 | AGI number: AT3G46590.1 uORF ′5 STOP: 100 uORF 3′ STOP: 202 leader length: 376 |
| 2214 | AGI number: AT3G46590.1 uORF ′5 STOP: 100 uORF 3′ STOP: 202 leader length: 376 |
| 2215 | AGI number: AT3G46590.2 uORF ′5 STOP: 100 uORF 3′ STOP: 202 leader length: 376 |
| 2216 | AGI number: AT3G46590.2 uORF ′5 STOP: 100 uORF 3′ STOP: 202 leader length: 376 |
| 2217 | AGI number: AT3G46590.2 uORF ′5 STOP: 120 uORF 3′ STOP: 171 leader length: 376 |
| 2218 | AGI number: AT3G46590.2 uORF ′5 STOP: 120 uORF 3′ STOP: 171 leader length: 376 |
| 2219 | AGI number: AT3G46590.3 uORF ′5 STOP: 100 uORF 3′ STOP: 202 leader length: 376 |
| 2220 | AGI number: AT3G46590.3 uORF ′5 STOP: 100 uORF 3′ STOP: 202 leader length: 376 |
| 2221 | AGI number: AT3G46590.3 uORF ′5 STOP: 120 uORF 3′ STOP: 171 leader length: 376 |
| 2222 | AGI number: AT3G46590.3 uORF ′5 STOP: 120 uORF 3′ STOP: 171 leader length: 376 |
| 2223 | AGI number: AT3G46930.1 uORF ′5 STOP: 252 uORF 3′ STOP: 378 leader length: 416 |
| 2224 | AGI number: AT3G46930.1 uORF ′5 STOP: 252 uORF 3′ STOP: 378 leader length: 416 |
| 2225 | AGI number: AT3G47390.1 uORF ′5 STOP: 180 uORF 3′ STOP: 231 leader length: 382 |
| 2226 | AGI number: AT3G47390.1 uORF ′5 STOP: 180 uORF 3′ STOP: 231 leader length: 382 |
| 2227 | AGI number: AT3G47390.2 uORF ′5 STOP: 180 uORF 3′ STOP: 231 leader length: 382 |
| 2228 | AGI number: AT3G47390.2 uORF ′5 STOP: 180 uORF 3′ STOP: 231 leader length: 382 |
| 2229 | AGI number: AT3G47550.2 uORF ′5 STOP: 88 uORF 3′ STOP: 163 leader length: 383 |
| 2230 | AGI number: AT3G47550.2 uORF ′5 STOP: 88 uORF 3′ STOP: 163 leader length: 383 |
| 2231 | AGI number: AT3G47550.2 uORF ′5 STOP: 93 uORF 3′ STOP: 159 leader length: 383 |
| 2232 | AGI number: AT3G47550.2 uORF ′5 STOP: 93 uORF 3′ STOP: 159 leader length: 383 |
| 2233 | AGI number: AT3G47550.6 uORF ′5 STOP: 53 uORF 3′ STOP: 119 leader length: 343 |
| 2234 | AGI number: AT3G47550.6 uORF ′5 STOP: 53 uORF 3′ STOP: 119 leader length: 343 |
| 2235 | AGI number: AT3G47680.1 uORF ′5 STOP: 302 uORF 3′ STOP: 386 leader length: 652 |
| 2236 | AGI number: AT3G47680.1 uORF ′5 STOP: 302 uORF 3′ STOP: 386 leader length: 652 |
| 2237 | AGI number: AT3G47965.1 uORF ′5 STOP: 41 uORF 3′ STOP: 110 leader length: 200 |
| 2238 | AGI number: AT3G47965.1 uORF ′5 STOP: 41 uORF 3′ STOP: 110 leader length: 200 |
| 2239 | AGI number: AT3G47965.1 uORF ′5 STOP: 60 uORF 3′ STOP: 123 leader length: 200 |
| 2240 | AGI number: AT3G47965.1 uORF ′5 STOP: 60 uORF 3′ STOP: 123 leader length: 200 |
| 2241 | AGI number: AT3G47965.1 uORF ′5 STOP: 110 uORF 3′ STOP: 218 leader length: 200 |
| 2242 | AGI number: AT3G47965.1 uORF ′5 STOP: 110 uORF 3′ STOP: 218 leader length: 200 |
| 2243 | AGI number: AT3G48060.1 uORF ′5 STOP: 261 uORF 3′ STOP: 315 leader length: 294 |
| 2244 | AGI number: AT3G48060.1 uORF ′5 STOP: 261 uORF 3′ STOP: 315 leader length: 294 |
| 2245 | AGI number: AT3G49050.1 uORF ′5 STOP: 6 uORF 3′ STOP: 213 leader length: 530 |
| 2246 | AGI number: AT3G49050.1 uORF ′5 STOP: 6 uORF 3′ STOP: 213 leader length: 530 |
| 2247 | AGI number: AT3G49050.1 uORF ′5 STOP: 106 uORF 3′ STOP: 202 leader length: 530 |
| 2248 | AGI number: AT3G49050.1 uORF ′5 STOP: 106 uORF 3′ STOP: 202 leader length: 530 |
| 2249 | AGI number: AT3G49290.2 uORF ′5 STOP: 150 uORF 3′ STOP: 204 leader length: 334 |
| 2250 | AGI number: AT3G49290.2 uORF ′5 STOP: 150 uORF 3′ STOP: 204 leader length: 334 |
| 2251 | AGI number: AT3G49430.3 uORF ′5 STOP: 109 uORF 3′ STOP: 268 leader length: 445 |
| 2252 | AGI number: AT3G49430.3 uORF ′5 STOP: 109 uORF 3′ STOP: 268 leader length: 445 |
| 2253 | AGI number: AT3G49690.1 uORF ′5 STOP: 23 uORF 3′ STOP: 98 leader length: 206 |
| 2254 | AGI number: AT3G49690.1 uORF ′5 STOP: 23 uORF 3′ STOP: 98 leader length: 206 |
| 2255 | AGI number: AT3G49725.1 uORF ′5 STOP: 11 uORF 3′ STOP: 65 leader length: 60 |
| 2256 | AGI number: AT3G49725.1 uORF ′5 STOP: 11 uORF 3′ STOP: 65 leader length: 60 |
| 2257 | AGI number: AT3G49810.1 uORF ′5 STOP: 180 uORF 3′ STOP: 306 leader length: 552 |
| 2258 | AGI number: AT3G49810.1 uORF ′5 STOP: 180 uORF 3′ STOP: 306 leader length: 552 |
| 2259 | AGI number: AT3G49810.1 uORF ′5 STOP: 254 uORF 3′ STOP: 320 leader length: 552 |
| 2260 | AGI number: AT3G49810.1 uORF ′5 STOP: 254 uORF 3′ STOP: 320 leader length: 552 |
| 2261 | AGI number: AT3G49845.1 uORF ′5 STOP: 62 uORF 3′ STOP: 473 leader length: 81 |
| 2262 | AGI number: AT3G49845.1 uORF ′5 STOP: 62 uORF 3′ STOP: 473 leader length: 81 |
| 2263 | AGI number: AT3G50700.1 uORF ′5 STOP: 377 uORF 3′ STOP: 440 leader length: 534 |
| 2264 | AGI number: AT3G50700.1 uORF ′5 STOP: 377 uORF 3′ STOP: 440 leader length: 534 |
| 2265 | AGI number: AT3G50860.1 uORF ′5 STOP: 64 uORF 3′ STOP: 121 leader length: 349 |
| 2266 | AGI number: AT3G50860.1 uORF ′5 STOP: 64 uORF 3′ STOP: 121 leader length: 349 |
| 2267 | AGI number: AT3G51040.2 uORF ′5 STOP: 331 uORF 3′ STOP: 436 leader length: 620 |
| 2268 | AGI number: AT3G51040.2 uORF ′5 STOP: 331 uORF 3′ STOP: 436 leader length: 620 |
| 2269 | AGI number: AT3G51040.3 uORF ′5 STOP: 108 uORF 3′ STOP: 180 leader length: 374 |
| 2270 | AGI number: AT3G51040.3 uORF ′5 STOP: 108 uORF 3′ STOP: 180 leader length: 374 |
| 2271 | AGI number: AT3G51140.1 uORF ′5 STOP: 26 uORF 3′ STOP: 98 leader length: 332 |
| 2272 | AGI number: AT3G51140.1 uORF ′5 STOP: 26 uORF 3′ STOP: 98 leader length: 332 |
| 2273 | AGI number: AT3G51180.1 uORF ′5 STOP: 52 uORF 3′ STOP: 247 leader length: 330 |
| 2274 | AGI number: AT3G51180.1 uORF ′5 STOP: 52 uORF 3′ STOP: 247 leader length: 330 |
| 2275 | AGI number: AT3G51290.2 uORF ′5 STOP: 118 uORF 3′ STOP: 298 leader length: 468 |
| 2276 | AGI number: AT3G51290.2 uORF ′5 STOP: 118 uORF 3′ STOP: 298 leader length: 468 |
| 2277 | AGI number: AT3G51300.1 uORF ′5 STOP: 69 uORF 3′ STOP: 222 leader length: 310 |
| 2278 | AGI number: AT3G51300.1 uORF ′5 STOP: 69 uORF 3′ STOP: 222 leader length: 310 |
| 2279 | AGI number: AT3G51370.1 uORF ′5 STOP: 118 uORF 3′ STOP: 295 leader length: 454 |
| 2280 | AGI number: AT3G51370.1 uORF ′5 STOP: 118 uORF 3′ STOP: 295 leader length: 454 |
| 2281 | AGI number: AT3G51370.2 uORF ′5 STOP: 436 uORF 3′ STOP: 544 leader length: 566 |
| 2282 | AGI number: AT3G51370.2 uORF ′5 STOP: 436 uORF 3′ STOP: 544 leader length: 566 |
| 2283 | AGI number: AT3G51370.2 uORF ′5 STOP: 228 uORF 3′ STOP: 564 leader length: 566 |
| 2284 | AGI number: AT3G51370.2 uORF ′5 STOP: 228 uORF 3′ STOP: 564 leader length: 566 |
| 2285 | AGI number: AT3G51480.1 uORF ′5 STOP: 25 uORF 3′ STOP: 112 leader length: 482 |
| 2286 | AGI number: AT3G51480.1 uORF ′5 STOP: 25 uORF 3′ STOP: 112 leader length: 482 |
| 2287 | AGI number: AT3G51480.1 uORF ′5 STOP: 242 uORF 3′ STOP: 302 leader length: 482 |
| 2288 | AGI number: AT3G51480.1 uORF ′5 STOP: 242 uORF 3′ STOP: 302 leader length: 482 |
| 2289 | AGI number: AT3G51630.1 uORF ′5 STOP: 289 uORF 3′ STOP: 481 leader length: 580 |
| 2290 | AGI number: AT3G51630.1 uORF ′5 STOP: 289 uORF 3′ STOP: 481 leader length: 580 |
| 2291 | AGI number: AT3G51950.1 uORF ′5 STOP: 321 uORF 3′ STOP: 372 leader length: 539 |
| 2292 | AGI number: AT3G51950.1 uORF ′5 STOP: 321 uORF 3′ STOP: 372 leader length: 539 |
| 2293 | AGI number: AT3G51950.1 uORF ′5 STOP: 203 uORF 3′ STOP: 314 leader length: 539 |
| 2294 | AGI number: AT3G51950.1 uORF ′5 STOP: 203 uORF 3′ STOP: 314 leader length: 539 |
| 2295 | AGI number: AT3G51950.2 uORF ′5 STOP: 183 uORF 3′ STOP: 294 leader length: 519 |
| 2296 | AGI number: AT3G51950.2 uORF ′5 STOP: 183 uORF 3′ STOP: 294 leader length: 519 |
| 2297 | AGI number: AT3G52170.2 uORF ′5 STOP: 26 uORF 3′ STOP: 92 leader length: 187 |
| 2298 | AGI number: AT3G52170.2 uORF ′5 STOP: 26 uORF 3′ STOP: 92 leader length: 187 |
| 2299 | AGI number: AT3G52290.1 uORF ′5 STOP: 119 uORF 3′ STOP: 203 leader length: 348 |
| 2300 | AGI number: AT3G52290.1 uORF ′5 STOP: 119 uORF 3′ STOP: 203 leader length: 348 |
| 2301 | AGI number: AT3G52730.1 uORF ′5 STOP: 33 uORF 3′ STOP: 309 leader length: 97 |
| 2302 | AGI number: AT3G52730.1 uORF ′5 STOP: 33 uORF 3′ STOP: 309 leader length: 97 |
| 2303 | AGI number: AT3G52880.2 uORF ′5 STOP: 6 uORF 3′ STOP: 150 leader length: 88 |
| 2304 | AGI number: AT3G52880.2 uORF ′5 STOP: 6 uORF 3′ STOP: 150 leader length: 88 |
| 2305 | AGI number: AT3G52890.1 uORF ′5 STOP: 348 uORF 3′ STOP: 456 leader length: 719 |
| 2306 | AGI number: AT3G52890.1 uORF ′5 STOP: 348 uORF 3′ STOP: 456 leader length: 719 |
| 2307 | AGI number: AT3G52990.2 uORF ′5 STOP: 66 uORF 3′ STOP: 240 leader length: 456 |
| 2308 | AGI number: AT3G52990.2 uORF ′5 STOP: 66 uORF 3′ STOP: 240 leader length: 456 |
| 2309 | AGI number: AT3G52990.2 uORF ′5 STOP: 137 uORF 3′ STOP: 275 leader length: 456 |
| 2310 | AGI number: AT3G52990.2 uORF ′5 STOP: 137 uORF 3′ STOP: 275 leader length: 456 |
| 2311 | AGI number: AT3G53270.2 uORF ′5 STOP: 217 uORF 3′ STOP: 304 leader length: 462 |
| 2312 | AGI number: AT3G53270.2 uORF ′5 STOP: 217 uORF 3′ STOP: 304 leader length: 462 |
| 2313 | AGI number: AT3G53270.2 uORF ′5 STOP: 236 uORF 3′ STOP: 299 leader length: 462 |
| 2314 | AGI number: AT3G53270.2 uORF ′5 STOP: 236 uORF 3′ STOP: 299 leader length: 462 |
| 2315 | AGI number: AT3G53500.1 uORF ′5 STOP: 153 uORF 3′ STOP: 285 leader length: 498 |
| 2316 | AGI number: AT3G53500.1 uORF ′5 STOP: 153 uORF 3′ STOP: 285 leader length: 498 |
| 2317 | AGI number: AT3G53500.1 uORF ′5 STOP: 300 uORF 3′ STOP: 351 leader length: 498 |
| 2318 | AGI number: AT3G53500.1 uORF ′5 STOP: 300 uORF 3′ STOP: 351 leader length: 498 |
| 2319 | AGI number: AT3G53500.1 uORF ′5 STOP: 62 uORF 3′ STOP: 323 leader length: 498 |
| 2320 | AGI number: AT3G53500.1 uORF ′5 STOP: 62 uORF 3′ STOP: 323 leader length: 498 |
| 2321 | AGI number: AT3G53668.1 uORF ′5 STOP: 49 uORF 3′ STOP: 124 leader length: 287 |
| 2322 | AGI number: AT3G53668.1 uORF ′5 STOP: 49 uORF 3′ STOP: 124 leader length: 287 |
| 2323 | AGI number: AT3G53668.1 uORF ′5 STOP: 57 uORF 3′ STOP: 135 leader length: 287 |
| 2324 | AGI number: AT3G53668.1 uORF ′5 STOP: 57 uORF 3′ STOP: 135 leader length: 287 |
| 2325 | AGI number: AT3G53670.1 uORF ′5 STOP: 174 uORF 3′ STOP: 414 leader length: 596 |
| 2326 | AGI number: AT3G53670.1 uORF ′5 STOP: 174 uORF 3′ STOP: 414 leader length: 596 |
| 2327 | AGI number: AT3G53670.1 uORF ′5 STOP: 57 uORF 3′ STOP: 135 leader length: 596 |
| 2328 | AGI number: AT3G53670.1 uORF ′5 STOP: 57 uORF 3′ STOP: 135 leader length: 596 |
| 2329 | AGI number: AT3G53670.1 uORF ′5 STOP: 49 uORF 3′ STOP: 124 leader length: 596 |
| 2330 | AGI number: AT3G53670.1 uORF ′5 STOP: 49 uORF 3′ STOP: 124 leader length: 596 |
| 2331 | AGI number: AT3G53670.2 uORF ′5 STOP: 106 uORF 3′ STOP: 181 leader length: 549 |
| 2332 | AGI number: AT3G53670.2 uORF ′5 STOP: 106 uORF 3′ STOP: 181 leader length: 549 |
| 2333 | AGI number: AT3G53670.2 uORF ′5 STOP: 114 uORF 3′ STOP: 192 leader length: 549 |
| 2334 | AGI number: AT3G53670.2 uORF ′5 STOP: 114 uORF 3′ STOP: 192 leader length: 549 |
| 2335 | AGI number: AT3G53850.1 uORF ′5 STOP: 93 uORF 3′ STOP: 186 leader length: 294 |
| 2336 | AGI number: AT3G53850.1 uORF ′5 STOP: 93 uORF 3′ STOP: 186 leader length: 294 |
| 2337 | AGI number: AT3G53850.1 uORF ′5 STOP: 89 uORF 3′ STOP: 206 leader length: 294 |
| 2338 | AGI number: AT3G53850.1 uORF ′5 STOP: 89 uORF 3′ STOP: 206 leader length: 294 |
| 2339 | AGI number: AT3G54010.2 uORF ′5 STOP: 250 uORF 3′ STOP: 316 leader length: 521 |
| 2340 | AGI number: AT3G54010.2 uORF ′5 STOP: 250 uORF 3′ STOP: 316 leader length: 521 |
| 2341 | AGI number: AT3G54190.1 uORF ′5 STOP: 79 uORF 3′ STOP: 157 leader length: 274 |
| 2342 | AGI number: AT3G54190.1 uORF ′5 STOP: 79 uORF 3′ STOP: 157 leader length: 274 |
| 2343 | AGI number: AT3G54220.1 uORF ′5 STOP: 278 uORF 3′ STOP: 440 leader length: 392 |
| 2344 | AGI number: AT3G54220.1 uORF ′5 STOP: 278 uORF 3′ STOP: 440 leader length: 392 |
| 2345 | AGI number: AT3G54350.3 uORF ′5 STOP: 22 uORF 3′ STOP: 157 leader length: 245 |
| 2346 | AGI number: AT3G54350.3 uORF ′5 STOP: 22 uORF 3′ STOP: 157 leader length: 245 |
| 2347 | AGI number: AT3G54350.3 uORF ′5 STOP: 44 uORF 3′ STOP: 161 leader length: 245 |
| 2348 | AGI number: AT3G54350.3 uORF ′5 STOP: 44 uORF 3′ STOP: 161 leader length: 245 |
| 2349 | AGI number: AT3G54380.3 uORF ′5 STOP: 257 uORF 3′ STOP: 464 leader length: 530 |
| 2350 | AGI number: AT3G54380.3 uORF ′5 STOP: 257 uORF 3′ STOP: 464 leader length: 530 |
| 2351 | AGI number: AT3G55020.1 uORF ′5 STOP: 47 uORF 3′ STOP: 125 leader length: 198 |
| 2352 | AGI number: AT3G55020.1 uORF ′5 STOP: 47 uORF 3′ STOP: 125 leader length: 198 |
| 2353 | AGI number: AT3G55050.1 uORF ′5 STOP: 102 uORF 3′ STOP: 303 leader length: 314 |
| 2354 | AGI number: AT3G55050.1 uORF ′5 STOP: 102 uORF 3′ STOP: 303 leader length: 314 |
| 2355 | AGI number: AT3G55080.2 uORF ′5 STOP: 60 uORF 3′ STOP: 258 leader length: 341 |
| 2356 | AGI number: AT3G55080.2 uORF ′5 STOP: 60 uORF 3′ STOP: 258 leader length: 341 |
| 2357 | AGI number: AT3G55080.2 uORF ′5 STOP: 161 uORF 3′ STOP: 269 leader length: 341 |
| 2358 | AGI number: AT3G55080.2 uORF ′5 STOP: 161 uORF 3′ STOP: 269 leader length: 341 |
| 2359 | AGI number: AT3G55140.2 uORF ′5 STOP: 69 uORF 3′ STOP: 168 leader length: 277 |
| 2360 | AGI number: AT3G55140.2 uORF ′5 STOP: 69 uORF 3′ STOP: 168 leader length: 277 |
| 2361 | AGI number: AT3G55140.2 uORF ′5 STOP: 83 uORF 3′ STOP: 179 leader length: 277 |
| 2362 | AGI number: AT3G55140.2 uORF ′5 STOP: 83 uORF 3′ STOP: 179 leader length: 277 |
| 2363 | AGI number: AT3G55480.1 uORF ′5 STOP: 328 uORF 3′ STOP: 379 leader length: 533 |
| 2364 | AGI number: AT3G55480.1 uORF ′5 STOP: 328 uORF 3′ STOP: 379 leader length: 533 |
| 2365 | AGI number: AT3G55560.1 uORF ′5 STOP: 147 uORF 3′ STOP: 222 leader length: 367 |
| 2366 | AGI number: AT3G55560.1 uORF ′5 STOP: 147 uORF 3′ STOP: 222 leader length: 367 |
| 2367 | AGI number: AT3G55560.1 uORF ′5 STOP: 133 uORF 3′ STOP: 217 leader length: 367 |
| 2368 | AGI number: AT3G55560.1 uORF ′5 STOP: 133 uORF 3′ STOP: 217 leader length: 367 |
| 2369 | AGI number: AT3G55560.1 uORF ′5 STOP: 158 uORF 3′ STOP: 230 leader length: 367 |
| 2370 | AGI number: AT3G55560.1 uORF ′5 STOP: 158 uORF 3′ STOP: 230 leader length: 367 |
| 2371 | AGI number: AT3G55820.1 uORF ′5 STOP: 311 uORF 3′ STOP: 458 leader length: 332 |
| 2372 | AGI number: AT3G55820.1 uORF ′5 STOP: 311 uORF 3′ STOP: 458 leader length: 332 |
| 2373 | AGI number: AT3G55850.2 uORF ′5 STOP: 40 uORF 3′ STOP: 130 leader length: 285 |
| 2374 | AGI number: AT3G55850.2 uORF ′5 STOP: 40 uORF 3′ STOP: 130 leader length: 285 |
| 2375 | AGI number: AT3G55850.2 uORF ′5 STOP: 66 uORF 3′ STOP: 135 leader length: 285 |
| 2376 | AGI number: AT3G55850.2 uORF ′5 STOP: 66 uORF 3′ STOP: 135 leader length: 285 |
| 2377 | AGI number: AT3G55860.1 uORF ′5 STOP: 39 uORF 3′ STOP: 120 leader length: 656 |
| 2378 | AGI number: AT3G55860.1 uORF ′5 STOP: 39 uORF 3′ STOP: 120 leader length: 656 |
| 2379 | AGI number: AT3G55860.1 uORF ′5 STOP: 5 uORF 3′ STOP: 104 leader length: 656 |
| 2380 | AGI number: AT3G55860.1 uORF ′5 STOP: 5 uORF 3′ STOP: 104 leader length: 656 |
| 2381 | AGI number: AT3G56130.2 uORF ′5 STOP: 9 uORF 3′ STOP: 510 leader length: 629 |
| 2382 | AGI number: AT3G56130.2 uORF ′5 STOP: 9 uORF 3′ STOP: 510 leader length: 629 |
| 2383 | AGI number: AT3G56130.2 uORF ′5 STOP: 416 uORF 3′ STOP: 503 leader length: 629 |
| 2384 | AGI number: AT3G56130.2 uORF ′5 STOP: 416 uORF 3′ STOP: 503 leader length: 629 |
| 2385 | AGI number: AT3G56130.2 uORF ′5 STOP: 394 uORF 3′ STOP: 454 leader length: 629 |
| 2386 | AGI number: AT3G56130.2 uORF ′5 STOP: 394 uORF 3′ STOP: 454 leader length: 629 |
| 2387 | AGI number: AT3G56130.3 uORF ′5 STOP: 416 uORF 3′ STOP: 503 leader length: 666 |
| 2388 | AGI number: AT3G56130.3 uORF ′5 STOP: 416 uORF 3′ STOP: 503 leader length: 666 |
| 2389 | AGI number: AT3G56130.3 uORF ′5 STOP: 9 uORF 3′ STOP: 510 leader length: 666 |
| 2390 | AGI number: AT3G56130.3 uORF ′5 STOP: 9 uORF 3′ STOP: 510 leader length: 666 |
| 2391 | AGI number: AT3G56130.3 uORF ′5 STOP: 394 uORF 3′ STOP: 454 leader length: 666 |
| 2392 | AGI number: AT3G56130.3 uORF ′5 STOP: 394 uORF 3′ STOP: 454 leader length: 666 |
| 2393 | AGI number: AT3G56270.1 uORF ′5 STOP: 129 uORF 3′ STOP: 216 leader length: 220 |
| 2394 | AGI number: AT3G56270.1 uORF ′5 STOP: 129 uORF 3′ STOP: 216 leader length: 220 |
| 2395 | AGI number: AT3G56580.1 uORF ′5 STOP: 12 uORF 3′ STOP: 237 leader length: 341 |
| 2396 | AGI number: AT3G56580.1 uORF ′5 STOP: 12 uORF 3′ STOP: 237 leader length: 341 |
| 2397 | AGI number: AT3G56580.1 uORF ′5 STOP: 160 uORF 3′ STOP: 274 leader length: 341 |
| 2398 | AGI number: AT3G56580.1 uORF ′5 STOP: 160 uORF 3′ STOP: 274 leader length: 341 |
| 2399 | AGI number: AT3G56580.2 uORF ′5 STOP: 77 uORF 3′ STOP: 128 leader length: 215 |
| 2400 | AGI number: AT3G56580.2 uORF ′5 STOP: 77 uORF 3′ STOP: 128 leader length: 215 |
| 2401 | AGI number: AT3G56850.1 uORF ′5 STOP: 327 uORF 3′ STOP: 498 leader length: 622 |
| 2402 | AGI number: AT3G56850.1 uORF ′5 STOP: 327 uORF 3′ STOP: 498 leader length: 622 |
| 2403 | AGI number: AT3G56850.1 uORF ′5 STOP: 466 uORF 3′ STOP: 544 leader length: 622 |
| 2404 | AGI number: AT3G56850.1 uORF ′5 STOP: 466 uORF 3′ STOP: 544 leader length: 622 |
| 2405 | AGI number: AT3G56850.1 uORF ′5 STOP: 410 uORF 3′ STOP: 509 leader length: 622 |
| 2406 | AGI number: AT3G56850.1 uORF ′5 STOP: 410 uORF 3′ STOP: 509 leader length: 622 |
| 2407 | AGI number: AT3G56850.1 uORF ′5 STOP: 108 uORF 3′ STOP: 198 leader length: 622 |
| 2408 | AGI number: AT3G56850.1 uORF ′5 STOP: 108 uORF 3′ STOP: 198 leader length: 622 |
| 2409 | AGI number: AT3G57630.1 uORF ′5 STOP: 34 uORF 3′ STOP: 136 leader length: 290 |
| 2410 | AGI number: AT3G57630.1 uORF ′5 STOP: 34 uORF 3′ STOP: 136 leader length: 290 |
| 2411 | AGI number: AT3G57790.1 uORF ′5 STOP: 30 uORF 3′ STOP: 177 leader length: 294 |
| 2412 | AGI number: AT3G57790.1 uORF ′5 STOP: 30 uORF 3′ STOP: 177 leader length: 294 |
| 2413 | AGI number: AT3G57790.1 uORF ′5 STOP: 101 uORF 3′ STOP: 164 leader length: 294 |
| 2414 | AGI number: AT3G57790.1 uORF ′5 STOP: 101 uORF 3′ STOP: 164 leader length: 294 |
| 2415 | AGI number: AT3G58030.2 uORF ′5 STOP: 226 uORF 3′ STOP: 361 leader length: 469 |
| 2416 | AGI number: AT3G58030.2 uORF ′5 STOP: 226 uORF 3′ STOP: 361 leader length: 469 |
| 2417 | AGI number: AT3G58510.2 uORF ′5 STOP: 73 uORF 3′ STOP: 127 leader length: 339 |
| 2418 | AGI number: AT3G58510.2 uORF ′5 STOP: 73 uORF 3′ STOP: 127 leader length: 339 |
| 2419 | AGI number: AT3G58520.2 uORF ′5 STOP: 8 uORF 3′ STOP: 119 leader length: 209 |
| 2420 | AGI number: AT3G58520.2 uORF ′5 STOP: 8 uORF 3′ STOP: 119 leader length: 209 |
| 2421 | AGI number: AT3G58560.1 uORF ′5 STOP: 117 uORF 3′ STOP: 177 leader length: 304 |
| 2422 | AGI number: AT3G58560.1 uORF ′5 STOP: 117 uORF 3′ STOP: 177 leader length: 304 |
| 2423 | AGI number: AT3G58650.1 uORF ′5 STOP: 259 uORF 3′ STOP: 334 leader length: 567 |
| 2424 | AGI number: AT3G58650.1 uORF ′5 STOP: 259 uORF 3′ STOP: 334 leader length: 567 |
| 2425 | AGI number: AT3G58650.1 uORF ′5 STOP: 267 uORF 3′ STOP: 339 leader length: 567 |
| 2426 | AGI number: AT3G58650.1 uORF ′5 STOP: 267 uORF 3′ STOP: 339 leader length: 567 |
| 2427 | AGI number: AT3G58670.3 uORF ′5 STOP: 29 uORF 3′ STOP: 143 leader length: 452 |
| 2428 | AGI number: AT3G58670.3 uORF ′5 STOP: 29 uORF 3′ STOP: 143 leader length: 452 |
| 2429 | AGI number: AT3G59470.1 uORF ′5 STOP: 98 uORF 3′ STOP: 167 leader length: 447 |
| 2430 | AGI number: AT3G59470.1 uORF ′5 STOP: 98 uORF 3′ STOP: 167 leader length: 447 |
| 2431 | AGI number: AT3G59470.1 uORF ′5 STOP: 270 uORF 3′ STOP: 333 leader length: 447 |
| 2432 | AGI number: AT3G59470.1 uORF ′5 STOP: 270 uORF 3′ STOP: 333 leader length: 447 |
| 2433 | AGI number: AT3G59570.1 uORF ′5 STOP: 213 uORF 3′ STOP: 423 leader length: 270 |
| 2434 | AGI number: AT3G59570.1 uORF ′5 STOP: 213 uORF 3′ STOP: 423 leader length: 270 |
| 2435 | AGI number: AT3G59570.1 uORF ′5 STOP: 9 uORF 3′ STOP: 204 leader length: 270 |
| 2436 | AGI number: AT3G59570.1 uORF ′5 STOP: 9 uORF 3′ STOP: 204 leader length: 270 |
| 2437 | AGI number: AT3G59770.2 uORF ′5 STOP: 559 uORF 3′ STOP: 751 leader length: 915 |
| 2438 | AGI number: AT3G59770.2 uORF ′5 STOP: 559 uORF 3′ STOP: 751 leader length: 915 |
| 2439 | AGI number: AT3G59770.2 uORF ′5 STOP: 111 uORF 3′ STOP: 759 leader length: 915 |
| 2440 | AGI number: AT3G59770.2 uORF ′5 STOP: 111 uORF 3′ STOP: 759 leader length: 915 |
| 2441 | AGI number: AT3G60240.4 uORF ′5 STOP: 19 uORF 3′ STOP: 106 leader length: 192 |
| 2442 | AGI number: AT3G60240.4 uORF ′5 STOP: 19 uORF 3′ STOP: 106 leader length: 192 |
| 2443 | AGI number: AT3G60240.4 uORF ′5 STOP: 29 uORF 3′ STOP: 98 leader length: 192 |
| 2444 | AGI number: AT3G60240.4 uORF ′5 STOP: 29 uORF 3′ STOP: 98 leader length: 192 |
| 2445 | AGI number: AT3G60540.1 uORF ′5 STOP: 17 uORF 3′ STOP: 122 leader length: 66 |
| 2446 | AGI number: AT3G60540.1 uORF ′5 STOP: 17 uORF 3′ STOP: 122 leader length: 66 |
| 2447 | AGI number: AT3G60540.2 uORF ′5 STOP: 439 uORF 3′ STOP: 544 leader length: 488 |
| 2448 | AGI number: AT3G60540.2 uORF ′5 STOP: 439 uORF 3′ STOP: 544 leader length: 488 |
| 2449 | AGI number: AT3G60540.2 uORF ′5 STOP: 479 uORF 3′ STOP: 710 leader length: 488 |
| 2450 | AGI number: AT3G60540.2 uORF ′5 STOP: 479 uORF 3′ STOP: 710 leader length: 488 |
| 2451 | AGI number: AT3G60800.1 uORF ′5 STOP: 28 uORF 3′ STOP: 130 leader length: 319 |
| 2452 | AGI number: AT3G60800.1 uORF ′5 STOP: 28 uORF 3′ STOP: 130 leader length: 319 |
| 2453 | AGI number: AT3G60800.1 uORF ′5 STOP: 15 uORF 3′ STOP: 168 leader length: 319 |
| 2454 | AGI number: AT3G60800.1 uORF ′5 STOP: 15 uORF 3′ STOP: 168 leader length: 319 |
| 2455 | AGI number: AT3G61160.1 uORF ′5 STOP: 69 uORF 3′ STOP: 168 leader length: 328 |
| 2456 | AGI number: AT3G61160.1 uORF ′5 STOP: 69 uORF 3′ STOP: 168 leader length: 328 |
| 2457 | AGI number: AT3G61160.2 uORF ′5 STOP: 11 uORF 3′ STOP: 110 leader length: 270 |
| 2458 | AGI number: AT3G61160.2 uORF ′5 STOP: 11 uORF 3′ STOP: 110 leader length: 270 |
| 2459 | AGI number: AT3G61160.2 uORF ′5 STOP: 66 uORF 3′ STOP: 144 leader length: 270 |
| 2460 | AGI number: AT3G61160.2 uORF ′5 STOP: 66 uORF 3′ STOP: 144 leader length: 270 |
| 2461 | AGI number: AT3G61160.2 uORF ′5 STOP: 107 uORF 3′ STOP: 182 leader length: 270 |
| 2462 | AGI number: AT3G61160.2 uORF ′5 STOP: 107 uORF 3′ STOP: 182 leader length: 270 |
| 2463 | AGI number: AT3G61250.1 uORF ′5 STOP: 117 uORF 3′ STOP: 219 leader length: 304 |
| 2464 | AGI number: AT3G61250.1 uORF ′5 STOP: 117 uORF 3′ STOP: 219 leader length: 304 |
| 2465 | AGI number: AT3G61250.1 uORF ′5 STOP: 61 uORF 3′ STOP: 208 leader length: 304 |
| 2466 | AGI number: AT3G61250.1 uORF ′5 STOP: 61 uORF 3′ STOP: 208 leader length: 304 |
| 2467 | AGI number: AT3G61310.1 uORF ′5 STOP: 33 uORF 3′ STOP: 111 leader length: 389 |
| 2468 | AGI number: AT3G61310.1 uORF ′5 STOP: 33 uORF 3′ STOP: 111 leader length: 389 |
| 2469 | AGI number: AT3G61530.2 uORF ′5 STOP: 9 uORF 3′ STOP: 318 leader length: 385 |
| 2470 | AGI number: AT3G61530.2 uORF ′5 STOP: 9 uORF 3′ STOP: 318 leader length: 385 |
| 2471 | AGI number: AT3G61530.2 uORF ′5 STOP: 248 uORF 3′ STOP: 326 leader length: 385 |
| 2472 | AGI number: AT3G61530.2 uORF ′5 STOP: 248 uORF 3′ STOP: 326 leader length: 385 |
| 2473 | AGI number: AT3G61550.1 uORF ′5 STOP: 22 uORF 3′ STOP: 112 leader length: 43 |
| 2474 | AGI number: AT3G61550.1 uORF ′5 STOP: 22 uORF 3′ STOP: 112 leader length: 43 |
| 2475 | AGI number: AT3G61590.1 uORF ′5 STOP: 126 uORF 3′ STOP: 264 leader length: 419 |
| 2476 | AGI number: AT3G61590.1 uORF ′5 STOP: 126 uORF 3′ STOP: 264 leader length: 419 |
| 2477 | AGI number: AT3G61590.1 uORF ′5 STOP: 214 uORF 3′ STOP: 286 leader length: 419 |
| 2478 | AGI number: AT3G61590.1 uORF ′5 STOP: 214 uORF 3′ STOP: 286 leader length: 419 |
| 2479 | AGI number: AT3G61620.2 uORF ′5 STOP: 86 uORF 3′ STOP: 215 leader length: 340 |
| 2480 | AGI number: AT3G61620.2 uORF ′5 STOP: 86 uORF 3′ STOP: 215 leader length: 340 |
| 2481 | AGI number: AT3G61680.1 uORF ′5 STOP: 120 uORF 3′ STOP: 273 leader length: 358 |
| 2482 | AGI number: AT3G61680.1 uORF ′5 STOP: 120 uORF 3′ STOP: 273 leader length: 358 |
| 2483 | AGI number: AT3G61680.1 uORF ′5 STOP: 97 uORF 3′ STOP: 265 leader length: 358 |
| 2484 | AGI number: AT3G61680.1 uORF ′5 STOP: 97 uORF 3′ STOP: 265 leader length: 358 |
| 2485 | AGI number: AT3G61830.1 uORF ′5 STOP: 53 uORF 3′ STOP: 185 leader length: 282 |
| 2486 | AGI number: AT3G61830.1 uORF ′5 STOP: 53 uORF 3′ STOP: 185 leader length: 282 |
| 2487 | AGI number: AT3G61850.2 uORF ′5 STOP: 41 uORF 3′ STOP: 332 leader length: 693 |
| 2488 | AGI number: AT3G61850.2 uORF ′5 STOP: 41 uORF 3′ STOP: 332 leader length: 693 |
| 2489 | AGI number: AT3G62020.2 uORF ′5 STOP: 5 uORF 3′ STOP: 122 leader length: 221 |
| 2490 | AGI number: AT3G62020.2 uORF ′5 STOP: 5 uORF 3′ STOP: 122 leader length: 221 |
| 2491 | AGI number: AT3G62190.2 uORF ′5 STOP: 54 uORF 3′ STOP: 291 leader length: 106 |
| 2492 | AGI number: AT3G62190.2 uORF ′5 STOP: 54 uORF 3′ STOP: 291 leader length: 106 |
| 2493 | AGI number: AT3G62270.1 uORF ′5 STOP: 222 uORF 3′ STOP: 288 leader length: 415 |
| 2494 | AGI number: AT3G62270.1 uORF ′5 STOP: 222 uORF 3′ STOP: 288 leader length: 415 |
| 2495 | AGI number: AT3G62270.1 uORF ′5 STOP: 164 uORF 3′ STOP: 254 leader length: 415 |
| 2496 | AGI number: AT3G62270.1 uORF ′5 STOP: 164 uORF 3′ STOP: 254 leader length: 415 |
| 2497 | AGI number: AT3G62270.1 uORF ′5 STOP: 235 uORF 3′ STOP: 301 leader length: 415 |
| 2498 | AGI number: AT3G62270.1 uORF ′5 STOP: 235 uORF 3′ STOP: 301 leader length: 415 |
| 2499 | AGI number: AT3G62400.1 uORF ′5 STOP: 77 uORF 3′ STOP: 281 leader length: 203 |
| 2500 | AGI number: AT3G62400.1 uORF ′5 STOP: 77 uORF 3′ STOP: 281 leader length: 203 |
| 2501 | AGI number: AT3G62400.2 uORF ′5 STOP: 66 uORF 3′ STOP: 270 leader length: 192 |
| 2502 | AGI number: AT3G62400.2 uORF ′5 STOP: 66 uORF 3′ STOP: 270 leader length: 192 |
| 2503 | AGI number: AT3G62420.1 uORF ′5 STOP: 291 uORF 3′ STOP: 390 leader length: 566 |
| 2504 | AGI number: AT3G62420.1 uORF ′5 STOP: 291 uORF 3′ STOP: 390 leader length: 566 |
| 2505 | AGI number: AT3G62420.1 uORF ′5 STOP: 299 uORF 3′ STOP: 419 leader length: 566 |
| 2506 | AGI number: AT3G62420.1 uORF ′5 STOP: 299 uORF 3′ STOP: 419 leader length: 566 |
| 2507 | AGI number: AT3G62420.1 uORF ′5 STOP: 268 uORF 3′ STOP: 385 leader length: 566 |
| 2508 | AGI number: AT3G62420.1 uORF ′5 STOP: 268 uORF 3′ STOP: 385 leader length: 566 |
| 2509 | AGI number: AT3G62422.1 uORF ′5 STOP: 291 uORF 3′ STOP: 390 leader length: 297 |
| 2510 | AGI number: AT3G62422.1 uORF ′5 STOP: 291 uORF 3′ STOP: 390 leader length: 297 |
| 2511 | AGI number: AT3G62660.1 uORF ′5 STOP: 126 uORF 3′ STOP: 246 leader length: 613 |
| 2512 | AGI number: AT3G62660.1 uORF ′5 STOP: 126 uORF 3′ STOP: 246 leader length: 613 |
| 2513 | AGI number: AT3G62660.1 uORF ′5 STOP: 146 uORF 3′ STOP: 278 leader length: 613 |
| 2514 | AGI number: AT3G62660.1 uORF ′5 STOP: 146 uORF 3′ STOP: 278 leader length: 613 |
| 2515 | AGI number: AT3G63050.1 uORF ′5 STOP: 4 uORF 3′ STOP: 73 leader length: 17 |
| 2516 | AGI number: AT3G63050.1 uORF ′5 STOP: 4 uORF 3′ STOP: 73 leader length: 17 |
| 2517 | AGI number: AT3G63080.1 uORF ′5 STOP: 115 uORF 3′ STOP: 217 leader length: 424 |
| 2518 | AGI number: AT3G63080.1 uORF ′5 STOP: 115 uORF 3′ STOP: 217 leader length: 424 |
| 2519 | AGI number: AT3G63130.1 uORF ′5 STOP: 43 uORF 3′ STOP: 127 leader length: 234 |
| 2520 | AGI number: AT3G63130.1 uORF ′5 STOP: 43 uORF 3′ STOP: 127 leader length: 234 |
| 2521 | AGI number: AT3G63130.2 uORF ′5 STOP: 55 uORF 3′ STOP: 139 leader length: 243 |
| 2522 | AGI number: AT3G63130.2 uORF ′5 STOP: 55 uORF 3′ STOP: 139 leader length: 243 |
| 2523 | AGI number: AT3G63210.1 uORF ′5 STOP: 167 uORF 3′ STOP: 230 leader length: 254 |
| 2524 | AGI number: AT3G63210.1 uORF ′5 STOP: 167 uORF 3′ STOP: 230 leader length: 254 |
| 2525 | AGI number: AT3G63240.1 uORF ′5 STOP: 24 uORF 3′ STOP: 255 leader length: 716 |
| 2526 | AGI number: AT3G63240.1 uORF ′5 STOP: 24 uORF 3′ STOP: 255 leader length: 716 |
| 2527 | AGI number: AT3G63250.2 uORF ′5 STOP: 94 uORF 3′ STOP: 202 leader length: 642 |
| 2528 | AGI number: AT3G63250.2 uORF ′5 STOP: 94 uORF 3′ STOP: 202 leader length: 642 |
| 2529 | AGI number: AT3G63250.2 uORF ′5 STOP: 47 uORF 3′ STOP: 209 leader length: 642 |
| 2530 | AGI number: AT3G63250.2 uORF ′5 STOP: 47 uORF 3′ STOP: 209 leader length: 642 |
| 2531 | AGI number: AT3G63450.3 uORF ′5 STOP: 25 uORF 3′ STOP: 109 leader length: 290 |
| 2532 | AGI number: AT3G63450.3 uORF ′5 STOP: 25 uORF 3′ STOP: 109 leader length: 290 |
| 2533 | AGI number: AT3G66654.2 uORF ′5 STOP: 173 uORF 3′ STOP: 245 leader length: 429 |
| 2534 | AGI number: AT3G66654.2 uORF ′5 STOP: 173 uORF 3′ STOP: 245 leader length: 429 |
| 2535 | AGI number: AT3G66654.2 uORF ′5 STOP: 187 uORF 3′ STOP: 238 leader length: 429 |
| 2536 | AGI number: AT3G66654.2 uORF ′5 STOP: 187 uORF 3′ STOP: 238 leader length: 429 |
| 2537 | AGI number: AT3G66654.3 uORF ′5 STOP: 97 uORF 3′ STOP: 229 leader length: 571 |
| 2538 | AGI number: AT3G66654.3 uORF ′5 STOP: 97 uORF 3′ STOP: 229 leader length: 571 |
| 2539 | AGI number: AT4G00110.1 uORF ′5 STOP: 40 uORF 3′ STOP: 100 leader length: 195 |
| 2540 | AGI number: AT4G00110.1 uORF ′5 STOP: 40 uORF 3′ STOP: 100 leader length: 195 |
| 2541 | AGI number: AT4G00130.1 uORF ′5 STOP: 74 uORF 3′ STOP: 134 leader length: 148 |
| 2542 | AGI number: AT4G00130.1 uORF ′5 STOP: 74 uORF 3′ STOP: 134 leader length: 148 |
| 2543 | AGI number: AT4G00180.2 uORF ′5 STOP: 276 uORF 3′ STOP: 414 leader length: 496 |
| 2544 | AGI number: AT4G00180.2 uORF ′5 STOP: 276 uORF 3′ STOP: 414 leader length: 496 |
| 2545 | AGI number: AT4G00390.1 uORF ′5 STOP: 39 uORF 3′ STOP: 150 leader length: 99 |
| 2546 | AGI number: AT4G00390.1 uORF ′5 STOP: 39 uORF 3′ STOP: 150 leader length: 99 |
| 2547 | AGI number: AT4G00440.1 uORF ′5 STOP: 358 uORF 3′ STOP: 490 leader length: 846 |
| 2548 | AGI number: AT4G00440.1 uORF ′5 STOP: 358 uORF 3′ STOP: 490 leader length: 846 |
| 2549 | AGI number: AT4G00440.1 uORF ′5 STOP: 470 uORF 3′ STOP: 728 leader length: 846 |
| 2550 | AGI number: AT4G00440.1 uORF ′5 STOP: 470 uORF 3′ STOP: 728 leader length: 846 |
| 2551 | AGI number: AT4G00440.1 uORF ′5 STOP: 598 uORF 3′ STOP: 721 leader length: 846 |
| 2552 | AGI number: AT4G00440.1 uORF ′5 STOP: 598 uORF 3′ STOP: 721 leader length: 846 |
| 2553 | AGI number: AT4G00440.2 uORF ′5 STOP: 145 uORF 3′ STOP: 238 leader length: 846 |
| 2554 | AGI number: AT4G00440.2 uORF ′5 STOP: 145 uORF 3′ STOP: 238 leader length: 846 |
| 2555 | AGI number: AT4G00440.2 uORF ′5 STOP: 299 uORF 3′ STOP: 473 leader length: 846 |
| 2556 | AGI number: AT4G00440.2 uORF ′5 STOP: 299 uORF 3′ STOP: 473 leader length: 846 |
| 2557 | AGI number: AT4G00440.2 uORF ′5 STOP: 358 uORF 3′ STOP: 490 leader length: 846 |
| 2558 | AGI number: AT4G00440.2 uORF ′5 STOP: 358 uORF 3′ STOP: 490 leader length: 846 |
| 2559 | AGI number: AT4G00440.3 uORF ′5 STOP: 299 uORF 3′ STOP: 473 leader length: 846 |
| 2560 | AGI number: AT4G00440.3 uORF ′5 STOP: 299 uORF 3′ STOP: 473 leader length: 846 |
| 2561 | AGI number: AT4G00440.3 uORF ′5 STOP: 358 uORF 3′ STOP: 490 leader length: 846 |
| 2562 | AGI number: AT4G00440.3 uORF ′5 STOP: 358 uORF 3′ STOP: 490 leader length: 846 |
| 2563 | AGI number: AT4G00440.3 uORF ′5 STOP: 414 uORF 3′ STOP: 525 leader length: 846 |
| 2564 | AGI number: AT4G00440.3 uORF ′5 STOP: 414 uORF 3′ STOP: 525 leader length: 846 |
| 2565 | AGI number: AT4G00500.1 uORF ′5 STOP: 91 uORF 3′ STOP: 151 leader length: 240 |
| 2566 | AGI number: AT4G00500.1 uORF ′5 STOP: 91 uORF 3′ STOP: 151 leader length: 240 |
| 2567 | AGI number: AT4G00500.2 uORF ′5 STOP: 68 uORF 3′ STOP: 128 leader length: 217 |
| 2568 | AGI number: AT4G00500.2 uORF ′5 STOP: 68 uORF 3′ STOP: 128 leader length: 217 |
| 2569 | AGI number: AT4G00550.1 uORF ′5 STOP: 87 uORF 3′ STOP: 147 leader length: 282 |
| 2570 | AGI number: AT4G00550.1 uORF ′5 STOP: 87 uORF 3′ STOP: 147 leader length: 282 |
| 2571 | AGI number: AT4G00710.1 uORF ′5 STOP: 303 uORF 3′ STOP: 417 leader length: 668 |
| 2572 | AGI number: AT4G00710.1 uORF ′5 STOP: 303 uORF 3′ STOP: 417 leader length: 668 |
| 2573 | AGI number: AT4G00710.1 uORF ′5 STOP: 368 uORF 3′ STOP: 446 leader length: 668 |
| 2574 | AGI number: AT4G00710.1 uORF ′5 STOP: 368 uORF 3′ STOP: 446 leader length: 668 |
| 2575 | AGI number: AT4G00730.1 uORF ′5 STOP: 90 uORF 3′ STOP: 243 leader length: 405 |
| 2576 | AGI number: AT4G00730.1 uORF ′5 STOP: 90 uORF 3′ STOP: 243 leader length: 405 |
| 2577 | AGI number: AT4G00955.1 uORF ′5 STOP: 36 uORF 3′ STOP: 108 leader length: 100 |
| 2578 | AGI number: AT4G00955.1 uORF ′5 STOP: 36 uORF 3′ STOP: 108 leader length: 100 |
| 2579 | AGI number: AT4G01090.1 uORF ′5 STOP: 158 uORF 3′ STOP: 209 leader length: 431 |
| 2580 | AGI number: AT4G01090.1 uORF ′5 STOP: 158 uORF 3′ STOP: 209 leader length: 431 |
| 2581 | AGI number: AT4G01210.1 uORF ′5 STOP: 181 uORF 3′ STOP: 238 leader length: 420 |
| 2582 | AGI number: AT4G01210.1 uORF ′5 STOP: 181 uORF 3′ STOP: 238 leader length: 420 |
| 2583 | AGI number: AT4G01290.1 uORF ′5 STOP: 387 uORF 3′ STOP: 438 leader length: 587 |
| 2584 | AGI number: AT4G01290.1 uORF ′5 STOP: 387 uORF 3′ STOP: 438 leader length: 587 |
| 2585 | AGI number: AT4G01290.2 uORF ′5 STOP: 386 uORF 3′ STOP: 437 leader length: 586 |
| 2586 | AGI number: AT4G01290.2 uORF ′5 STOP: 386 uORF 3′ STOP: 437 leader length: 586 |
| 2587 | AGI number: AT4G02010.1 uORF ′5 STOP: 121 uORF 3′ STOP: 229 leader length: 367 |
| 2588 | AGI number: AT4G02010.1 uORF ′5 STOP: 121 uORF 3′ STOP: 229 leader length: 367 |
| 2589 | AGI number: AT4G02010.1 uORF ′5 STOP: 134 uORF 3′ STOP: 218 leader length: 367 |
| 2590 | AGI number: AT4G02010.1 uORF ′5 STOP: 134 uORF 3′ STOP: 218 leader length: 367 |
| 2591 | AGI number: AT4G02140.1 uORF ′5 STOP: 53 uORF 3′ STOP: 215 leader length: 213 |
| 2592 | AGI number: AT4G02140.1 uORF ′5 STOP: 53 uORF 3′ STOP: 215 leader length: 213 |
| 2593 | AGI number: AT4G02170.1 uORF ′5 STOP: 68 uORF 3′ STOP: 128 leader length: 77 |
| 2594 | AGI number: AT4G02170.1 uORF ′5 STOP: 68 uORF 3′ STOP: 128 leader length: 77 |
| 2595 | AGI number: AT4G02550.2 uORF ′5 STOP: 362 uORF 3′ STOP: 416 leader length: 368 |
| 2596 | AGI number: AT4G02550.2 uORF ′5 STOP: 362 uORF 3′ STOP: 416 leader length: 368 |
| 2597 | AGI number: AT4G02570.1 uORF ′5 STOP: 40 uORF 3′ STOP: 103 leader length: 269 |
| 2598 | AGI number: AT4G02570.1 uORF ′5 STOP: 40 uORF 3′ STOP: 103 leader length: 269 |
| 2599 | AGI number: AT4G02710.1 uORF ′5 STOP: 36 uORF 3′ STOP: 168 leader length: 340 |
| 2600 | AGI number: AT4G02710.1 uORF ′5 STOP: 36 uORF 3′ STOP: 168 leader length: 340 |
| 2601 | AGI number: AT4G02710.1 uORF ′5 STOP: 79 uORF 3′ STOP: 157 leader length: 340 |
| 2602 | AGI number: AT4G02710.1 uORF ′5 STOP: 79 uORF 3′ STOP: 157 leader length: 340 |
| 2603 | AGI number: AT4G02710.1 uORF ′5 STOP: 125 uORF 3′ STOP: 179 leader length: 340 |
| 2604 | AGI number: AT4G02710.1 uORF ′5 STOP: 125 uORF 3′ STOP: 179 leader length: 340 |
| 2605 | AGI number: AT4G02760.1 uORF ′5 STOP: 175 uORF 3′ STOP: 286 leader length: 433 |
| 2606 | AGI number: AT4G02760.1 uORF ′5 STOP: 175 uORF 3′ STOP: 286 leader length: 433 |
| 2607 | AGI number: AT4G02760.3 uORF ′5 STOP: 201 uORF 3′ STOP: 402 leader length: 213 |
| 2608 | AGI number: AT4G02760.3 uORF ′5 STOP: 201 uORF 3′ STOP: 402 leader length: 213 |
| 2609 | AGI number: AT4G03000.2 uORF ′5 STOP: 65 uORF 3′ STOP: 194 leader length: 466 |
| 2610 | AGI number: AT4G03000.2 uORF ′5 STOP: 65 uORF 3′ STOP: 194 leader length: 466 |
| 2611 | AGI number: AT4G03090.1 uORF ′5 STOP: 140 uORF 3′ STOP: 200 leader length: 278 |
| 2612 | AGI number: AT4G03090.1 uORF ′5 STOP: 140 uORF 3′ STOP: 200 leader length: 278 |
| 2613 | AGI number: AT4G03200.2 uORF ′5 STOP: 385 uORF 3′ STOP: 436 leader length: 710 |
| 2614 | AGI number: AT4G03200.2 uORF ′5 STOP: 385 uORF 3′ STOP: 436 leader length: 710 |
| 2615 | AGI number: AT4G03205.2 uORF ′5 STOP: 25 uORF 3′ STOP: 145 leader length: 29 |
| 2616 | AGI number: AT4G03205.2 uORF ′5 STOP: 25 uORF 3′ STOP: 145 leader length: 29 |
| 2617 | AGI number: AT4G03260.1 uORF ′5 STOP: 168 uORF 3′ STOP: 387 leader length: 577 |
| 2618 | AGI number: AT4G03260.1 uORF ′5 STOP: 168 uORF 3′ STOP: 387 leader length: 577 |
| 2619 | AGI number: AT4G03260.1 uORF ′5 STOP: 325 uORF 3′ STOP: 418 leader length: 577 |
| 2620 | AGI number: AT4G03260.1 uORF ′5 STOP: 325 uORF 3′ STOP: 418 leader length: 577 |
| 2621 | AGI number: AT4G03260.2 uORF ′5 STOP: 10 uORF 3′ STOP: 229 leader length: 324 |
| 2622 | AGI number: AT4G03260.2 uORF ′5 STOP: 10 uORF 3′ STOP: 229 leader length: 324 |
| 2623 | AGI number: AT4G03280.2 uORF ′5 STOP: 99 uORF 3′ STOP: 183 leader length: 285 |
| 2624 | AGI number: AT4G03280.2 uORF ′5 STOP: 99 uORF 3′ STOP: 183 leader length: 285 |
| 2625 | AGI number: AT4G03410.1 uORF ′5 STOP: 403 uORF 3′ STOP: 556 leader length: 739 |
| 2626 | AGI number: AT4G03410.1 uORF ′5 STOP: 403 uORF 3′ STOP: 556 leader length: 739 |
| 2627 | AGI number: AT4G03410.1 uORF ′5 STOP: 203 uORF 3′ STOP: 308 leader length: 739 |
| 2628 | AGI number: AT4G03410.1 uORF ′5 STOP: 203 uORF 3′ STOP: 308 leader length: 739 |
| 2629 | AGI number: AT4G03410.1 uORF ′5 STOP: 423 uORF 3′ STOP: 477 leader length: 739 |
| 2630 | AGI number: AT4G03410.1 uORF ′5 STOP: 423 uORF 3′ STOP: 477 leader length: 739 |
| 2631 | AGI number: AT4G03410.2 uORF ′5 STOP: 403 uORF 3′ STOP: 556 leader length: 739 |
| 2632 | AGI number: AT4G03410.2 uORF ′5 STOP: 403 uORF 3′ STOP: 556 leader length: 739 |
| 2633 | AGI number: AT4G03410.2 uORF ′5 STOP: 423 uORF 3′ STOP: 477 leader length: 739 |
| 2634 | AGI number: AT4G03410.2 uORF ′5 STOP: 423 uORF 3′ STOP: 477 leader length: 739 |
| 2635 | AGI number: AT4G03500.1 uORF ′5 STOP: 168 uORF 3′ STOP: 363 leader length: 361 |
| 2636 | AGI number: AT4G03500.1 uORF ′5 STOP: 168 uORF 3′ STOP: 363 leader length: 361 |
| 2637 | AGI number: AT4G03510.2 uORF ′5 STOP: 150 uORF 3′ STOP: 204 leader length: 722 |
| 2638 | AGI number: AT4G03510.2 uORF ′5 STOP: 150 uORF 3′ STOP: 204 leader length: 722 |
| 2639 | AGI number: AT4G04340.3 uORF ′5 STOP: 43 uORF 3′ STOP: 208 leader length: 547 |
| 2640 | AGI number: AT4G04340.3 uORF ′5 STOP: 43 uORF 3′ STOP: 208 leader length: 547 |
| 2641 | AGI number: AT4G04340.3 uORF ′5 STOP: 182 uORF 3′ STOP: 248 leader length: 547 |
| 2642 | AGI number: AT4G04340.3 uORF ′5 STOP: 182 uORF 3′ STOP: 248 leader length: 547 |
| 2643 | AGI number: AT4G04340.3 uORF ′5 STOP: 338 uORF 3′ STOP: 443 leader length: 547 |
| 2644 | AGI number: AT4G04340.3 uORF ′5 STOP: 338 uORF 3′ STOP: 443 leader length: 547 |
| 2645 | AGI number: AT4G04740.1 uORF ′5 STOP: 243 uORF 3′ STOP: 315 leader length: 244 |
| 2646 | AGI number: AT4G04740.1 uORF ′5 STOP: 243 uORF 3′ STOP: 315 leader length: 244 |
| 2647 | AGI number: AT4G04890.1 uORF ′5 STOP: 263 uORF 3′ STOP: 356 leader length: 448 |
| 2648 | AGI number: AT4G04890.1 uORF ′5 STOP: 263 uORF 3′ STOP: 356 leader length: 448 |
| 2649 | AGI number: AT4G06634.2 uORF ′5 STOP: 70 uORF 3′ STOP: 295 leader length: 314 |
| 2650 | AGI number: AT4G06634.2 uORF ′5 STOP: 70 uORF 3′ STOP: 295 leader length: 314 |
| 2651 | AGI number: AT4G07990.1 uORF ′5 STOP: 171 uORF 3′ STOP: 297 leader length: 199 |
| 2652 | AGI number: AT4G07990.1 uORF ′5 STOP: 171 uORF 3′ STOP: 297 leader length: 199 |
| 2653 | AGI number: AT4G08700.1 uORF ′5 STOP: 5 uORF 3′ STOP: 80 leader length: 172 |
| 2654 | AGI number: AT4G08700.1 uORF ′5 STOP: 5 uORF 3′ STOP: 80 leader length: 172 |
| 2655 | AGI number: AT4G08980.2 uORF ′5 STOP: 338 uORF 3′ STOP: 470 leader length: 589 |
| 2656 | AGI number: AT4G08980.2 uORF ′5 STOP: 338 uORF 3′ STOP: 470 leader length: 589 |
| 2657 | AGI number: AT4G08980.2 uORF ′5 STOP: 429 uORF 3′ STOP: 483 leader length: 589 |
| 2658 | AGI number: AT4G08980.2 uORF ′5 STOP: 429 uORF 3′ STOP: 483 leader length: 589 |
| 2659 | AGI number: AT4G09640.1 uORF ′5 STOP: 88 uORF 3′ STOP: 163 leader length: 284 |
| 2660 | AGI number: AT4G09640.1 uORF ′5 STOP: 88 uORF 3′ STOP: 163 leader length: 284 |
| 2661 | AGI number: AT4G10390.1 uORF ′5 STOP: 15 uORF 3′ STOP: 114 leader length: 274 |
| 2662 | AGI number: AT4G10390.1 uORF ′5 STOP: 15 uORF 3′ STOP: 114 leader length: 274 |
| 2663 | AGI number: AT4G10955.1 uORF ′5 STOP: 847 uORF 3′ STOP: 1057 leader length: 1475 |
| 2664 | AGI number: AT4G10955.1 uORF ′5 STOP: 847 uORF 3′ STOP: 1057 leader length: 1475 |
| 2665 | AGI number: AT4G10955.1 uORF ′5 STOP: 947 uORF 3′ STOP: 1067 leader length: 1475 |
| 2666 | AGI number: AT4G10955.1 uORF ′5 STOP: 947 uORF 3′ STOP: 1067 leader length: 1475 |
| 2667 | AGI number: AT4G11440.1 uORF ′5 STOP: 16 uORF 3′ STOP: 82 leader length: 151 |
| 2668 | AGI number: AT4G11440.1 uORF ′5 STOP: 16 uORF 3′ STOP: 82 leader length: 151 |
| 2669 | AGI number: AT4G11450.1 uORF ′5 STOP: 56 uORF 3′ STOP: 125 leader length: 317 |
| 2670 | AGI number: AT4G11450.1 uORF ′5 STOP: 56 uORF 3′ STOP: 125 leader length: 317 |
| 2671 | AGI number: AT4G11570.2 uORF ′5 STOP: 179 uORF 3′ STOP: 245 leader length: 408 |
| 2672 | AGI number: AT4G11570.2 uORF ′5 STOP: 179 uORF 3′ STOP: 245 leader length: 408 |
| 2673 | AGI number: AT4G11570.2 uORF ′5 STOP: 187 uORF 3′ STOP: 241 leader length: 408 |
| 2674 | AGI number: AT4G11570.2 uORF ′5 STOP: 187 uORF 3′ STOP: 241 leader length: 408 |
| 2675 | AGI number: AT4G11800.1 uORF ′5 STOP: 155 uORF 3′ STOP: 221 leader length: 374 |
| 2676 | AGI number: AT4G11800.1 uORF ′5 STOP: 155 uORF 3′ STOP: 221 leader length: 374 |
| 2677 | AGI number: AT4G11830.1 uORF ′5 STOP: 134 uORF 3′ STOP: 404 leader length: 1096 |
| 2678 | AGI number: AT4G11830.1 uORF ′5 STOP: 134 uORF 3′ STOP: 404 leader length: 1096 |
| 2679 | AGI number: AT4G11830.1 uORF ′5 STOP: 348 uORF 3′ STOP: 411 leader length: 1096 |
| 2680 | AGI number: AT4G11830.1 uORF ′5 STOP: 348 uORF 3′ STOP: 411 leader length: 1096 |
| 2681 | AGI number: AT4G11830.1 uORF ′5 STOP: 944 uORF 3′ STOP: 1007 leader length: 1096 |
| 2682 | AGI number: AT4G11830.1 uORF ′5 STOP: 944 uORF 3′ STOP: 1007 leader length: 1096 |
| 2683 | AGI number: AT4G11830.2 uORF ′5 STOP: 134 uORF 3′ STOP: 404 leader length: 1096 |
| 2684 | AGI number: AT4G11830.2 uORF ′5 STOP: 134 uORF 3′ STOP: 404 leader length: 1096 |
| 2685 | AGI number: AT4G11830.2 uORF ′5 STOP: 348 uORF 3′ STOP: 411 leader length: 1096 |
| 2686 | AGI number: AT4G11830.2 uORF ′5 STOP: 348 uORF 3′ STOP: 411 leader length: 1096 |
| 2687 | AGI number: AT4G11960.2 uORF ′5 STOP: 120 uORF 3′ STOP: 243 leader length: 193 |
| 2688 | AGI number: AT4G11960.2 uORF ′5 STOP: 120 uORF 3′ STOP: 243 leader length: 193 |
| 2689 | AGI number: AT4G12040.2 uORF ′5 STOP: 149 uORF 3′ STOP: 200 leader length: 631 |
| 2690 | AGI number: AT4G12040.2 uORF ′5 STOP: 149 uORF 3′ STOP: 200 leader length: 631 |
| 2691 | AGI number: AT4G12040.2 uORF ′5 STOP: 106 uORF 3′ STOP: 175 leader length: 631 |
| 2692 | AGI number: AT4G12040.2 uORF ′5 STOP: 106 uORF 3′ STOP: 175 leader length: 631 |
| 2693 | AGI number: AT4G12120.1 uORF ′5 STOP: 136 uORF 3′ STOP: 211 leader length: 266 |
| 2694 | AGI number: AT4G12120.1 uORF ′5 STOP: 136 uORF 3′ STOP: 211 leader length: 266 |
| 2695 | AGI number: AT4G12420.1 uORF ′5 STOP: 1 uORF 3′ STOP: 109 leader length: 206 |
| 2696 | AGI number: AT4G12420.1 uORF ′5 STOP: 1 uORF 3′ STOP: 109 leader length: 206 |
| 2697 | AGI number: AT4G12420.2 uORF ′5 STOP: 62 uORF 3′ STOP: 170 leader length: 267 |
| 2698 | AGI number: AT4G12420.2 uORF ′5 STOP: 62 uORF 3′ STOP: 170 leader length: 267 |
| 2699 | AGI number: AT4G12750.1 uORF ′5 STOP: 151 uORF 3′ STOP: 244 leader length: 311 |
| 2700 | AGI number: AT4G12750.1 uORF ′5 STOP: 151 uORF 3′ STOP: 244 leader length: 311 |
| 2701 | AGI number: AT4G12990.1 uORF ′5 STOP: 6 uORF 3′ STOP: 123 leader length: 129 |
| 2702 | AGI number: AT4G12990.1 uORF ′5 STOP: 6 uORF 3′ STOP: 123 leader length: 129 |
| 2703 | AGI number: AT4G13020.5 uORF ′5 STOP: 319 uORF 3′ STOP: 424 leader length: 507 |
| 2704 | AGI number: AT4G13020.5 uORF ′5 STOP: 319 uORF 3′ STOP: 424 leader length: 507 |
| 2705 | AGI number: AT4G13100.1 uORF ′5 STOP: 109 uORF 3′ STOP: 181 leader length: 329 |
| 2706 | AGI number: AT4G13100.1 uORF ′5 STOP: 109 uORF 3′ STOP: 181 leader length: 329 |
| 2707 | AGI number: AT4G13100.3 uORF ′5 STOP: 109 uORF 3′ STOP: 181 leader length: 329 |
| 2708 | AGI number: AT4G13100.3 uORF ′5 STOP: 109 uORF 3′ STOP: 181 leader length: 329 |
| 2709 | AGI number: AT4G13100.5 uORF ′5 STOP: 105 uORF 3′ STOP: 177 leader length: 330 |
| 2710 | AGI number: AT4G13100.5 uORF ′5 STOP: 105 uORF 3′ STOP: 177 leader length: 330 |
| 2711 | AGI number: AT4G13100.5 uORF ′5 STOP: 137 uORF 3′ STOP: 200 leader length: 330 |
| 2712 | AGI number: AT4G13100.5 uORF ′5 STOP: 137 uORF 3′ STOP: 200 leader length: 330 |
| 2713 | AGI number: AT4G13100.5 uORF ′5 STOP: 154 uORF 3′ STOP: 217 leader length: 330 |
| 2714 | AGI number: AT4G13100.5 uORF ′5 STOP: 154 uORF 3′ STOP: 217 leader length: 330 |
| 2715 | AGI number: AT4G13260.1 uORF ′5 STOP: 51 uORF 3′ STOP: 105 leader length: 373 |
| 2716 | AGI number: AT4G13260.1 uORF ′5 STOP: 51 uORF 3′ STOP: 105 leader length: 373 |
| 2717 | AGI number: AT4G13260.1 uORF ′5 STOP: 80 uORF 3′ STOP: 296 leader length: 373 |
| 2718 | AGI number: AT4G13260.1 uORF ′5 STOP: 80 uORF 3′ STOP: 296 leader length: 373 |
| 2719 | AGI number: AT4G13260.1 uORF ′5 STOP: 232 uORF 3′ STOP: 301 leader length: 373 |
| 2720 | AGI number: AT4G13260.1 uORF ′5 STOP: 232 uORF 3′ STOP: 301 leader length: 373 |
| 2721 | AGI number: AT4G13345.1 uORF ′5 STOP: 499 uORF 3′ STOP: 667 leader length: 548 |
| 2722 | AGI number: AT4G13345.1 uORF ′5 STOP: 499 uORF 3′ STOP: 667 leader length: 548 |
| 2723 | AGI number: AT4G13345.2 uORF ′5 STOP: 451 uORF 3′ STOP: 619 leader length: 500 |
| 2724 | AGI number: AT4G13345.2 uORF ′5 STOP: 451 uORF 3′ STOP: 619 leader length: 500 |
| 2725 | AGI number: AT4G13550.1 uORF ′5 STOP: 259 uORF 3′ STOP: 487 leader length: 629 |
| 2726 | AGI number: AT4G13550.1 uORF ′5 STOP: 259 uORF 3′ STOP: 487 leader length: 629 |
| 2727 | AGI number: AT4G14350.1 uORF ′5 STOP: 133 uORF 3′ STOP: 193 leader length: 308 |
| 2728 | AGI number: AT4G14350.1 uORF ′5 STOP: 133 uORF 3′ STOP: 193 leader length: 308 |
| 2729 | AGI number: AT4G14350.1 uORF ′5 STOP: 162 uORF 3′ STOP: 225 leader length: 308 |
| 2730 | AGI number: AT4G14350.1 uORF ′5 STOP: 162 uORF 3′ STOP: 225 leader length: 308 |
| 2731 | AGI number: AT4G14350.2 uORF ′5 STOP: 258 uORF 3′ STOP: 321 leader length: 404 |
| 2732 | AGI number: AT4G14350.2 uORF ′5 STOP: 258 uORF 3′ STOP: 321 leader length: 404 |
| 2733 | AGI number: AT4G14350.3 uORF ′5 STOP: 258 uORF 3′ STOP: 321 leader length: 404 |
| 2734 | AGI number: AT4G14350.3 uORF ′5 STOP: 258 uORF 3′ STOP: 321 leader length: 404 |
| 2735 | AGI number: AT4G14520.3 uORF ′5 STOP: 120 uORF 3′ STOP: 195 leader length: 312 |
| 2736 | AGI number: AT4G14520.3 uORF ′5 STOP: 120 uORF 3′ STOP: 195 leader length: 312 |
| 2737 | AGI number: AT4G14620.1 uORF ′5 STOP: 146 uORF 3′ STOP: 299 leader length: 385 |
| 2738 | AGI number: AT4G14620.1 uORF ′5 STOP: 146 uORF 3′ STOP: 299 leader length: 385 |
| 2739 | AGI number: AT4G14620.1 uORF ′5 STOP: 210 uORF 3′ STOP: 294 leader length: 385 |
| 2740 | AGI number: AT4G14620.1 uORF ′5 STOP: 210 uORF 3′ STOP: 294 leader length: 385 |
| 2741 | AGI number: AT4G14710.5 uORF ′5 STOP: 4 uORF 3′ STOP: 136 leader length: 230 |
| 2742 | AGI number: AT4G14710.5 uORF ′5 STOP: 4 uORF 3′ STOP: 136 leader length: 230 |
| 2743 | AGI number: AT4G14850.1 uORF ′5 STOP: 10 uORF 3′ STOP: 373 leader length: 88 |
| 2744 | AGI number: AT4G14850.1 uORF ′5 STOP: 10 uORF 3′ STOP: 373 leader length: 88 |
| 2745 | AGI number: AT4G14880.3 uORF ′5 STOP: 79 uORF 3′ STOP: 178 leader length: 291 |
| 2746 | AGI number: AT4G14880.3 uORF ′5 STOP: 79 uORF 3′ STOP: 178 leader length: 291 |
| 2747 | AGI number: AT4G14880.4 uORF ′5 STOP: 79 uORF 3′ STOP: 178 leader length: 294 |
| 2748 | AGI number: AT4G14880.4 uORF ′5 STOP: 79 uORF 3′ STOP: 178 leader length: 294 |
| 2749 | AGI number: AT4G15010.2 uORF ′5 STOP: 83 uORF 3′ STOP: 158 leader length: 362 |
| 2750 | AGI number: AT4G15010.2 uORF ′5 STOP: 83 uORF 3′ STOP: 158 leader length: 362 |
| 2751 | AGI number: AT4G15080.1 uORF ′5 STOP: 35 uORF 3′ STOP: 158 leader length: 351 |
| 2752 | AGI number: AT4G15080.1 uORF ′5 STOP: 35 uORF 3′ STOP: 158 leader length: 351 |
| 2753 | AGI number: AT4G15530.3 uORF ′5 STOP: 166 uORF 3′ STOP: 223 leader length: 452 |
| 2754 | AGI number: AT4G15530.3 uORF ′5 STOP: 166 uORF 3′ STOP: 223 leader length: 452 |
| 2755 | AGI number: AT4G16070.2 uORF ′5 STOP: 134 uORF 3′ STOP: 257 leader length: 387 |
| 2756 | AGI number: AT4G16070.2 uORF ′5 STOP: 134 uORF 3′ STOP: 257 leader length: 387 |
| 2757 | AGI number: AT4G16070.2 uORF ′5 STOP: 196 uORF 3′ STOP: 250 leader length: 387 |
| 2758 | AGI number: AT4G16070.2 uORF ′5 STOP: 196 uORF 3′ STOP: 250 leader length: 387 |
| 2759 | AGI number: AT4G16070.2 uORF ′5 STOP: 269 uORF 3′ STOP: 323 leader length: 387 |
| 2760 | AGI number: AT4G16070.2 uORF ′5 STOP: 269 uORF 3′ STOP: 323 leader length: 387 |
| 2761 | AGI number: AT4G16280.2 uORF ′5 STOP: 92 uORF 3′ STOP: 170 leader length: 413 |
| 2762 | AGI number: AT4G16280.2 uORF ′5 STOP: 92 uORF 3′ STOP: 170 leader length: 413 |
| 2763 | AGI number: AT4G16360.1 uORF ′5 STOP: 115 uORF 3′ STOP: 250 leader length: 463 |
| 2764 | AGI number: AT4G16360.1 uORF ′5 STOP: 115 uORF 3′ STOP: 250 leader length: 463 |
| 2765 | AGI number: AT4G16360.2 uORF ′5 STOP: 5 uORF 3′ STOP: 107 leader length: 540 |
| 2766 | AGI number: AT4G16360.2 uORF ′5 STOP: 5 uORF 3′ STOP: 107 leader length: 540 |
| 2767 | AGI number: AT4G16360.2 uORF ′5 STOP: 75 uORF 3′ STOP: 132 leader length: 540 |
| 2768 | AGI number: AT4G16360.2 uORF ′5 STOP: 75 uORF 3′ STOP: 132 leader length: 540 |
| 2769 | AGI number: AT4G16360.2 uORF ′5 STOP: 192 uORF 3′ STOP: 327 leader length: 540 |
| 2770 | AGI number: AT4G16360.2 uORF ′5 STOP: 192 uORF 3′ STOP: 327 leader length: 540 |
| 2771 | AGI number: AT4G16360.3 uORF ′5 STOP: 49 uORF 3′ STOP: 151 leader length: 493 |
| 2772 | AGI number: AT4G16360.3 uORF ′5 STOP: 49 uORF 3′ STOP: 151 leader length: 493 |
| 2773 | AGI number: AT4G16360.3 uORF ′5 STOP: 236 uORF 3′ STOP: 371 leader length: 493 |
| 2774 | AGI number: AT4G16360.3 uORF ′5 STOP: 236 uORF 3′ STOP: 371 leader length: 493 |
| 2775 | AGI number: AT4G16520.2 uORF ′5 STOP: 37 uORF 3′ STOP: 91 leader length: 329 |
| 2776 | AGI number: AT4G16520.2 uORF ′5 STOP: 37 uORF 3′ STOP: 91 leader length: 329 |
| 2777 | AGI number: AT4G16520.2 uORF ′5 STOP: 12 uORF 3′ STOP: 111 leader length: 329 |
| 2778 | AGI number: AT4G16520.2 uORF ′5 STOP: 12 uORF 3′ STOP: 111 leader length: 329 |
| 2779 | AGI number: AT4G16765.2 uORF ′5 STOP: 28 uORF 3′ STOP: 211 leader length: 310 |
| 2780 | AGI number: AT4G16765.2 uORF ′5 STOP: 28 uORF 3′ STOP: 211 leader length: 310 |
| 2781 | AGI number: AT4G16765.2 uORF ′5 STOP: 102 uORF 3′ STOP: 192 leader length: 310 |
| 2782 | AGI number: AT4G16765.2 uORF ′5 STOP: 102 uORF 3′ STOP: 192 leader length: 310 |
| 2783 | AGI number: AT4G16845.1 uORF ′5 STOP: 89 uORF 3′ STOP: 149 leader length: 232 |
| 2784 | AGI number: AT4G16845.1 uORF ′5 STOP: 89 uORF 3′ STOP: 149 leader length: 232 |
| 2785 | AGI number: AT4G16845.2 uORF ′5 STOP: 288 uORF 3′ STOP: 342 leader length: 588 |
| 2786 | AGI number: AT4G16845.2 uORF ′5 STOP: 288 uORF 3′ STOP: 342 leader length: 588 |
| 2787 | AGI number: AT4G16845.2 uORF ′5 STOP: 212 uORF 3′ STOP: 353 leader length: 588 |
| 2788 | AGI number: AT4G16845.2 uORF ′5 STOP: 212 uORF 3′ STOP: 353 leader length: 588 |
| 2789 | AGI number: AT4G16845.2 uORF ′5 STOP: 77 uORF 3′ STOP: 137 leader length: 588 |
| 2790 | AGI number: AT4G16845.2 uORF ′5 STOP: 77 uORF 3′ STOP: 137 leader length: 588 |
| 2791 | AGI number: AT4G16845.2 uORF ′5 STOP: 483 uORF 3′ STOP: 609 leader length: 588 |
| 2792 | AGI number: AT4G16845.2 uORF ′5 STOP: 483 uORF 3′ STOP: 609 leader length: 588 |
| 2793 | AGI number: AT4G16940.1 uORF ′5 STOP: 57 uORF 3′ STOP: 114 leader length: 94 |
| 2794 | AGI number: AT4G16940.1 uORF ′5 STOP: 57 uORF 3′ STOP: 114 leader length: 94 |
| 2795 | AGI number: AT4G16990.4 uORF ′5 STOP: 290 uORF 3′ STOP: 350 leader length: 671 |
| 2796 | AGI number: AT4G16990.4 uORF ′5 STOP: 290 uORF 3′ STOP: 350 leader length: 671 |
| 2797 | AGI number: AT4G16990.5 uORF ′5 STOP: 331 uORF 3′ STOP: 439 leader length: 671 |
| 2798 | AGI number: AT4G16990.5 uORF ′5 STOP: 331 uORF 3′ STOP: 439 leader length: 671 |
| 2799 | AGI number: AT4G17020.1 uORF ′5 STOP: 74 uORF 3′ STOP: 206 leader length: 310 |
| 2800 | AGI number: AT4G17020.1 uORF ′5 STOP: 74 uORF 3′ STOP: 206 leader length: 310 |
| 2801 | AGI number: AT4G17020.2 uORF ′5 STOP: 74 uORF 3′ STOP: 206 leader length: 310 |
| 2802 | AGI number: AT4G17020.2 uORF ′5 STOP: 74 uORF 3′ STOP: 206 leader length: 310 |
| 2803 | AGI number: AT4G17020.2 uORF ′5 STOP: 81 uORF 3′ STOP: 189 leader length: 310 |
| 2804 | AGI number: AT4G17020.2 uORF ′5 STOP: 81 uORF 3′ STOP: 189 leader length: 310 |
| 2805 | AGI number: AT4G17140.1 uORF ′5 STOP: 66 uORF 3′ STOP: 147 leader length: 255 |
| 2806 | AGI number: AT4G17140.1 uORF ′5 STOP: 66 uORF 3′ STOP: 147 leader length: 255 |
| 2807 | AGI number: AT4G17140.2 uORF ′5 STOP: 66 uORF 3′ STOP: 147 leader length: 255 |
| 2808 | AGI number: AT4G17140.2 uORF ′5 STOP: 66 uORF 3′ STOP: 147 leader length: 255 |
| 2809 | AGI number: AT4G17140.3 uORF ′5 STOP: 66 uORF 3′ STOP: 147 leader length: 255 |
| 2810 | AGI number: AT4G17140.3 uORF ′5 STOP: 66 uORF 3′ STOP: 147 leader length: 255 |
| 2811 | AGI number: AT4G17410.1 uORF ′5 STOP: 225 uORF 3′ STOP: 348 leader length: 586 |
| 2812 | AGI number: AT4G17410.1 uORF ′5 STOP: 225 uORF 3′ STOP: 348 leader length: 586 |
| 2813 | AGI number: AT4G17410.1 uORF ′5 STOP: 239 uORF 3′ STOP: 353 leader length: 586 |
| 2814 | AGI number: AT4G17410.1 uORF ′5 STOP: 239 uORF 3′ STOP: 353 leader length: 586 |
| 2815 | AGI number: AT4G17410.2 uORF ′5 STOP: 238 uORF 3′ STOP: 361 leader length: 439 |
| 2816 | AGI number: AT4G17410.2 uORF ′5 STOP: 238 uORF 3′ STOP: 361 leader length: 439 |
| 2817 | AGI number: AT4G17410.3 uORF ′5 STOP: 238 uORF 3′ STOP: 361 leader length: 599 |
| 2818 | AGI number: AT4G17410.3 uORF ′5 STOP: 238 uORF 3′ STOP: 361 leader length: 599 |
| 2819 | AGI number: AT4G17410.3 uORF ′5 STOP: 252 uORF 3′ STOP: 366 leader length: 599 |
| 2820 | AGI number: AT4G17410.3 uORF ′5 STOP: 252 uORF 3′ STOP: 366 leader length: 599 |
| 2821 | AGI number: AT4G18020.1 uORF ′5 STOP: 92 uORF 3′ STOP: 377 leader length: 144 |
| 2822 | AGI number: AT4G18020.1 uORF ′5 STOP: 92 uORF 3′ STOP: 377 leader length: 144 |
| 2823 | AGI number: AT4G18020.6 uORF ′5 STOP: 34 uORF 3′ STOP: 160 leader length: 307 |
| 2824 | AGI number: AT4G18020.6 uORF ′5 STOP: 34 uORF 3′ STOP: 160 leader length: 307 |
| 2825 | AGI number: AT4G18020.6 uORF ′5 STOP: 74 uORF 3′ STOP: 191 leader length: 307 |
| 2826 | AGI number: AT4G18020.6 uORF ′5 STOP: 74 uORF 3′ STOP: 191 leader length: 307 |
| 2827 | AGI number: AT4G18880.1 uORF ′5 STOP: 211 uORF 3′ STOP: 277 leader length: 511 |
| 2828 | AGI number: AT4G18880.1 uORF ′5 STOP: 211 uORF 3′ STOP: 277 leader length: 511 |
| 2829 | AGI number: AT4G19110.1 uORF ′5 STOP: 176 uORF 3′ STOP: 440 leader length: 938 |
| 2830 | AGI number: AT4G19110.1 uORF ′5 STOP: 176 uORF 3′ STOP: 440 leader length: 938 |
| 2831 | AGI number: AT4G19110.1 uORF ′5 STOP: 280 uORF 3′ STOP: 412 leader length: 938 |
| 2832 | AGI number: AT4G19110.1 uORF ′5 STOP: 280 uORF 3′ STOP: 412 leader length: 938 |
| 2833 | AGI number: AT4G19110.1 uORF ′5 STOP: 558 uORF 3′ STOP: 615 leader length: 938 |
| 2834 | AGI number: AT4G19110.1 uORF ′5 STOP: 558 uORF 3′ STOP: 615 leader length: 938 |
| 2835 | AGI number: AT4G19110.2 uORF ′5 STOP: 146 uORF 3′ STOP: 410 leader length: 908 |
| 2836 | AGI number: AT4G19110.2 uORF ′5 STOP: 146 uORF 3′ STOP: 410 leader length: 908 |
| 2837 | AGI number: AT4G19110.2 uORF ′5 STOP: 250 uORF 3′ STOP: 382 leader length: 908 |
| 2838 | AGI number: AT4G19110.2 uORF ′5 STOP: 250 uORF 3′ STOP: 382 leader length: 908 |
| 2839 | AGI number: AT4G19112.1 uORF ′5 STOP: 176 uORF 3′ STOP: 440 leader length: 285 |
| 2840 | AGI number: AT4G19112.1 uORF ′5 STOP: 176 uORF 3′ STOP: 440 leader length: 285 |
| 2841 | AGI number: AT4G19160.1 uORF ′5 STOP: 150 uORF 3′ STOP: 390 leader length: 915 |
| 2842 | AGI number: AT4G19160.1 uORF ′5 STOP: 150 uORF 3′ STOP: 390 leader length: 915 |
| 2843 | AGI number: AT4G19160.1 uORF ′5 STOP: 299 uORF 3′ STOP: 356 leader length: 915 |
| 2844 | AGI number: AT4G19160.1 uORF ′5 STOP: 299 uORF 3′ STOP: 356 leader length: 915 |
| 2845 | AGI number: AT4G19160.2 uORF ′5 STOP: 150 uORF 3′ STOP: 390 leader length: 466 |
| 2846 | AGI number: AT4G19160.2 uORF ′5 STOP: 150 uORF 3′ STOP: 390 leader length: 466 |
| 2847 | AGI number: AT4G19160.2 uORF ′5 STOP: 299 uORF 3′ STOP: 356 leader length: 466 |
| 2848 | AGI number: AT4G19160.2 uORF ′5 STOP: 299 uORF 3′ STOP: 356 leader length: 466 |
| 2849 | AGI number: AT4G19550.2 uORF ′5 STOP: 136 uORF 3′ STOP: 277 leader length: 187 |
| 2850 | AGI number: AT4G19550.2 uORF ′5 STOP: 136 uORF 3′ STOP: 277 leader length: 187 |
| 2851 | AGI number: AT4G19645.1 uORF ′5 STOP: 36 uORF 3′ STOP: 87 leader length: 161 |
| 2852 | AGI number: AT4G19645.1 uORF ′5 STOP: 36 uORF 3′ STOP: 87 leader length: 161 |
| 2853 | AGI number: AT4G20325.1 uORF ′5 STOP: 53 uORF 3′ STOP: 107 leader length: 184 |
| 2854 | AGI number: AT4G20325.1 uORF ′5 STOP: 53 uORF 3′ STOP: 107 leader length: 184 |
| 2855 | AGI number: AT4G20325.2 uORF ′5 STOP: 53 uORF 3′ STOP: 107 leader length: 184 |
| 2856 | AGI number: AT4G20325.2 uORF ′5 STOP: 53 uORF 3′ STOP: 107 leader length: 184 |
| 2857 | AGI number: AT4G21060.2 uORF ′5 STOP: 7 uORF 3′ STOP: 127 leader length: 477 |
| 2858 | AGI number: AT4G21060.2 uORF ′5 STOP: 7 uORF 3′ STOP: 127 leader length: 477 |
| 2859 | AGI number: AT4G21060.2 uORF ′5 STOP: 62 uORF 3′ STOP: 122 leader length: 477 |
| 2860 | AGI number: AT4G21060.2 uORF ′5 STOP: 62 uORF 3′ STOP: 122 leader length: 477 |
| 2861 | AGI number: AT4G21060.2 uORF ′5 STOP: 238 uORF 3′ STOP: 379 leader length: 477 |
| 2862 | AGI number: AT4G21060.2 uORF ′5 STOP: 238 uORF 3′ STOP: 379 leader length: 477 |
| 2863 | AGI number: AT4G21270.1 uORF ′5 STOP: 58 uORF 3′ STOP: 115 leader length: 251 |
| 2864 | AGI number: AT4G21270.1 uORF ′5 STOP: 58 uORF 3′ STOP: 115 leader length: 251 |
| 2865 | AGI number: AT4G21720.2 uORF ′5 STOP: 73 uORF 3′ STOP: 208 leader length: 219 |
| 2866 | AGI number: AT4G21720.2 uORF ′5 STOP: 73 uORF 3′ STOP: 208 leader length: 219 |
| 2867 | AGI number: AT4G21750.1 uORF ′5 STOP: 234 uORF 3′ STOP: 285 leader length: 365 |
| 2868 | AGI number: AT4G21750.1 uORF ′5 STOP: 234 uORF 3′ STOP: 285 leader length: 365 |
| 2869 | AGI number: AT4G21750.2 uORF ′5 STOP: 313 uORF 3′ STOP: 373 leader length: 466 |
| 2870 | AGI number: AT4G21750.2 uORF ′5 STOP: 313 uORF 3′ STOP: 373 leader length: 466 |
| 2871 | AGI number: AT4G21750.2 uORF ′5 STOP: 317 uORF 3′ STOP: 386 leader length: 466 |
| 2872 | AGI number: AT4G21750.2 uORF ′5 STOP: 317 uORF 3′ STOP: 386 leader length: 466 |
| 2873 | AGI number: AT4G21850.2 uORF ′5 STOP: 96 uORF 3′ STOP: 381 leader length: 100 |
| 2874 | AGI number: AT4G21850.2 uORF ′5 STOP: 96 uORF 3′ STOP: 381 leader length: 100 |
| 2875 | AGI number: AT4G22140.1 uORF ′5 STOP: 22 uORF 3′ STOP: 82 leader length: 279 |
| 2876 | AGI number: AT4G22140.1 uORF ′5 STOP: 22 uORF 3′ STOP: 82 leader length: 279 |
| 2877 | AGI number: AT4G22140.2 uORF ′5 STOP: 22 uORF 3′ STOP: 82 leader length: 279 |
| 2878 | AGI number: AT4G22140.2 uORF ′5 STOP: 22 uORF 3′ STOP: 82 leader length: 279 |
| 2879 | AGI number: AT4G22150.1 uORF ′5 STOP: 50 uORF 3′ STOP: 125 leader length: 101 |
| 2880 | AGI number: AT4G22150.1 uORF ′5 STOP: 50 uORF 3′ STOP: 125 leader length: 101 |
| 2881 | AGI number: AT4G22285.1 uORF ′5 STOP: 156 uORF 3′ STOP: 231 leader length: 362 |
| 2882 | AGI number: AT4G22285.1 uORF ′5 STOP: 156 uORF 3′ STOP: 231 leader length: 362 |
| 2883 | AGI number: AT4G22305.1 uORF ′5 STOP: 15 uORF 3′ STOP: 231 leader length: 136 |
| 2884 | AGI number: AT4G22305.1 uORF ′5 STOP: 15 uORF 3′ STOP: 231 leader length: 136 |
| 2885 | AGI number: AT4G22810.1 uORF ′5 STOP: 50 uORF 3′ STOP: 185 leader length: 292 |
| 2886 | AGI number: AT4G22810.1 uORF ′5 STOP: 50 uORF 3′ STOP: 185 leader length: 292 |
| 2887 | AGI number: AT4G22810.1 uORF ′5 STOP: 118 uORF 3′ STOP: 181 leader length: 292 |
| 2888 | AGI number: AT4G22810.1 uORF ′5 STOP: 118 uORF 3′ STOP: 181 leader length: 292 |
| 2889 | AGI number: AT4G22860.2 uORF ′5 STOP: 44 uORF 3′ STOP: 233 leader length: 352 |
| 2890 | AGI number: AT4G22860.2 uORF ′5 STOP: 44 uORF 3′ STOP: 233 leader length: 352 |
| 2891 | AGI number: AT4G22970.2 uORF ′5 STOP: 128 uORF 3′ STOP: 308 leader length: 156 |
| 2892 | AGI number: AT4G22970.2 uORF ′5 STOP: 128 uORF 3′ STOP: 308 leader length: 156 |
| 2893 | AGI number: AT4G22990.1 uORF ′5 STOP: 14 uORF 3′ STOP: 164 leader length: 277 |
| 2894 | AGI number: AT4G22990.1 uORF ′5 STOP: 14 uORF 3′ STOP: 164 leader length: 277 |
| 2895 | AGI number: AT4G22990.2 uORF ′5 STOP: 14 uORF 3′ STOP: 164 leader length: 277 |
| 2896 | AGI number: AT4G22990.2 uORF ′5 STOP: 14 uORF 3′ STOP: 164 leader length: 277 |
| 2897 | AGI number: AT4G22990.2 uORF ′5 STOP: 123 uORF 3′ STOP: 195 leader length: 277 |
| 2898 | AGI number: AT4G22990.2 uORF ′5 STOP: 123 uORF 3′ STOP: 195 leader length: 277 |
| 2899 | AGI number: AT4G23000.1 uORF ′5 STOP: 689 uORF 3′ STOP: 857 leader length: 786 |
| 2900 | AGI number: AT4G23000.1 uORF ′5 STOP: 689 uORF 3′ STOP: 857 leader length: 786 |
| 2901 | AGI number: AT4G23020.2 uORF ′5 STOP: 87 uORF 3′ STOP: 186 leader length: 96 |
| 2902 | AGI number: AT4G23020.2 uORF ′5 STOP: 87 uORF 3′ STOP: 186 leader length: 96 |
| 2903 | AGI number: AT4G23420.1 uORF ′5 STOP: 92 uORF 3′ STOP: 182 leader length: 681 |
| 2904 | AGI number: AT4G23420.1 uORF ′5 STOP: 92 uORF 3′ STOP: 182 leader length: 681 |
| 2905 | AGI number: AT4G23420.1 uORF ′5 STOP: 612 uORF 3′ STOP: 759 leader length: 681 |
| 2906 | AGI number: AT4G23420.1 uORF ′5 STOP: 612 uORF 3′ STOP: 759 leader length: 681 |
| 2907 | AGI number: AT4G23420.2 uORF ′5 STOP: 93 uORF 3′ STOP: 183 leader length: 548 |
| 2908 | AGI number: AT4G23420.2 uORF ′5 STOP: 93 uORF 3′ STOP: 183 leader length: 548 |
| 2909 | AGI number: AT4G23420.3 uORF ′5 STOP: 184 uORF 3′ STOP: 274 leader length: 545 |
| 2910 | AGI number: AT4G23420.3 uORF ′5 STOP: 184 uORF 3′ STOP: 274 leader length: 545 |
| 2911 | AGI number: AT4G23420.3 uORF ′5 STOP: 68 uORF 3′ STOP: 293 leader length: 545 |
| 2912 | AGI number: AT4G23420.3 uORF ′5 STOP: 68 uORF 3′ STOP: 293 leader length: 545 |
| 2913 | AGI number: AT4G23470.2 uORF ′5 STOP: 133 uORF 3′ STOP: 364 leader length: 734 |
| 2914 | AGI number: AT4G23470.2 uORF ′5 STOP: 133 uORF 3′ STOP: 364 leader length: 734 |
| 2915 | AGI number: AT4G23470.2 uORF ′5 STOP: 236 uORF 3′ STOP: 380 leader length: 734 |
| 2916 | AGI number: AT4G23470.2 uORF ′5 STOP: 236 uORF 3′ STOP: 380 leader length: 734 |
| 2917 | AGI number: AT4G23470.2 uORF ′5 STOP: 288 uORF 3′ STOP: 375 leader length: 734 |
| 2918 | AGI number: AT4G23470.2 uORF ′5 STOP: 288 uORF 3′ STOP: 375 leader length: 734 |
| 2919 | AGI number: AT4G23740.1 uORF ′5 STOP: 54 uORF 3′ STOP: 126 leader length: 294 |
| 2920 | AGI number: AT4G23740.1 uORF ′5 STOP: 54 uORF 3′ STOP: 126 leader length: 294 |
| 2921 | AGI number: AT4G23770.1 uORF ′5 STOP: 267 uORF 3′ STOP: 318 leader length: 446 |
| 2922 | AGI number: AT4G23770.1 uORF ′5 STOP: 267 uORF 3′ STOP: 318 leader length: 446 |
| 2923 | AGI number: AT4G23860.2 uORF ′5 STOP: 9 uORF 3′ STOP: 114 leader length: 200 |
| 2924 | AGI number: AT4G23860.2 uORF ′5 STOP: 9 uORF 3′ STOP: 114 leader length: 200 |
| 2925 | AGI number: AT4G23860.2 uORF ′5 STOP: 44 uORF 3′ STOP: 107 leader length: 200 |
| 2926 | AGI number: AT4G23860.2 uORF ′5 STOP: 44 uORF 3′ STOP: 107 leader length: 200 |
| 2927 | AGI number: AT4G24480.1 uORF ′5 STOP: 217 uORF 3′ STOP: 355 leader length: 423 |
| 2928 | AGI number: AT4G24480.1 uORF ′5 STOP: 217 uORF 3′ STOP: 355 leader length: 423 |
| 2929 | AGI number: AT4G24670.2 uORF ′5 STOP: 19 uORF 3′ STOP: 115 leader length: 233 |
| 2930 | AGI number: AT4G24670.2 uORF ′5 STOP: 19 uORF 3′ STOP: 115 leader length: 233 |
| 2931 | AGI number: AT4G24960.2 uORF ′5 STOP: 23 uORF 3′ STOP: 182 leader length: 344 |
| 2932 | AGI number: AT4G24960.2 uORF ′5 STOP: 23 uORF 3′ STOP: 182 leader length: 344 |
| 2933 | AGI number: AT4G25100.1 uORF ′5 STOP: 7 uORF 3′ STOP: 79 leader length: 194 |
| 2934 | AGI number: AT4G25100.1 uORF ′5 STOP: 7 uORF 3′ STOP: 79 leader length: 194 |
| 2935 | AGI number: AT4G25320.1 uORF ′5 STOP: 137 uORF 3′ STOP: 218 leader length: 411 |
| 2936 | AGI number: AT4G25320.1 uORF ′5 STOP: 137 uORF 3′ STOP: 218 leader length: 411 |
| 2937 | AGI number: AT4G25320.1 uORF ′5 STOP: 151 uORF 3′ STOP: 223 leader length: 411 |
| 2938 | AGI number: AT4G25320.1 uORF ′5 STOP: 151 uORF 3′ STOP: 223 leader length: 411 |
| 2939 | AGI number: AT4G25320.1 uORF ′5 STOP: 171 uORF 3′ STOP: 228 leader length: 411 |
| 2940 | AGI number: AT4G25320.1 uORF ′5 STOP: 171 uORF 3′ STOP: 228 leader length: 411 |
| 2941 | AGI number: AT4G25390.2 uORF ′5 STOP: 72 uORF 3′ STOP: 249 leader length: 150 |
| 2942 | AGI number: AT4G25390.2 uORF ′5 STOP: 72 uORF 3′ STOP: 249 leader length: 150 |
| 2943 | AGI number: AT4G25670.1 uORF ′5 STOP: 168 uORF 3′ STOP: 363 leader length: 433 |
| 2944 | AGI number: AT4G25670.1 uORF ′5 STOP: 168 uORF 3′ STOP: 363 leader length: 433 |
| 2945 | AGI number: AT4G25670.2 uORF ′5 STOP: 1 uORF 3′ STOP: 184 leader length: 254 |
| 2946 | AGI number: AT4G25670.2 uORF ′5 STOP: 1 uORF 3′ STOP: 184 leader length: 254 |
| 2947 | AGI number: AT4G25690.1 uORF ′5 STOP: 52 uORF 3′ STOP: 277 leader length: 802 |
| 2948 | AGI number: AT4G25690.1 uORF ′5 STOP: 52 uORF 3′ STOP: 277 leader length: 802 |
| 2949 | AGI number: AT4G25690.1 uORF ′5 STOP: 239 uORF 3′ STOP: 296 leader length: 802 |
| 2950 | AGI number: AT4G25690.1 uORF ′5 STOP: 239 uORF 3′ STOP: 296 leader length: 802 |
| 2951 | AGI number: AT4G25690.1 uORF ′5 STOP: 551 uORF 3′ STOP: 728 leader length: 802 |
| 2952 | AGI number: AT4G25690.1 uORF ′5 STOP: 551 uORF 3′ STOP: 728 leader length: 802 |
| 2953 | AGI number: AT4G25690.1 uORF ′5 STOP: 606 uORF 3′ STOP: 699 leader length: 802 |
| 2954 | AGI number: AT4G25690.1 uORF ′5 STOP: 606 uORF 3′ STOP: 699 leader length: 802 |
| 2955 | AGI number: AT4G25690.2 uORF ′5 STOP: 51 uORF 3′ STOP: 258 leader length: 332 |
| 2956 | AGI number: AT4G25690.2 uORF ′5 STOP: 51 uORF 3′ STOP: 258 leader length: 332 |
| 2957 | AGI number: AT4G25690.2 uORF ′5 STOP: 136 uORF 3′ STOP: 229 leader length: 332 |
| 2958 | AGI number: AT4G25690.2 uORF ′5 STOP: 136 uORF 3′ STOP: 229 leader length: 332 |
| 2959 | AGI number: AT4G25692.1 uORF ′5 STOP: 56 uORF 3′ STOP: 125 leader length: 558 |
| 2960 | AGI number: AT4G25692.1 uORF ′5 STOP: 56 uORF 3′ STOP: 125 leader length: 558 |
| 2961 | AGI number: AT4G25692.1 uORF ′5 STOP: 51 uORF 3′ STOP: 276 leader length: 558 |
| 2962 | AGI number: AT4G25692.1 uORF ′5 STOP: 51 uORF 3′ STOP: 276 leader length: 558 |
| 2963 | AGI number: AT4G25692.1 uORF ′5 STOP: 238 uORF 3′ STOP: 295 leader length: 558 |
| 2964 | AGI number: AT4G25692.1 uORF ′5 STOP: 238 uORF 3′ STOP: 295 leader length: 558 |
| 2965 | AGI number: AT4G25692.1 uORF ′5 STOP: 292 uORF 3′ STOP: 343 leader length: 558 |
| 2966 | AGI number: AT4G25692.1 uORF ′5 STOP: 292 uORF 3′ STOP: 343 leader length: 558 |
| 2967 | AGI number: AT4G26100.1 uORF ′5 STOP: 19 uORF 3′ STOP: 124 leader length: 289 |
| 2968 | AGI number: AT4G26100.1 uORF ′5 STOP: 19 uORF 3′ STOP: 124 leader length: 289 |
| 2969 | AGI number: AT4G26270.1 uORF ′5 STOP: 33 uORF 3′ STOP: 132 leader length: 317 |
| 2970 | AGI number: AT4G26270.1 uORF ′5 STOP: 33 uORF 3′ STOP: 132 leader length: 317 |
| 2971 | AGI number: AT4G26320.1 uORF ′5 STOP: 50 uORF 3′ STOP: 269 leader length: 56 |
| 2972 | AGI number: AT4G26320.1 uORF ′5 STOP: 50 uORF 3′ STOP: 269 leader length: 56 |
| 2973 | AGI number: AT4G26520.1 uORF ′5 STOP: 68 uORF 3′ STOP: 152 leader length: 290 |
| 2974 | AGI number: AT4G26520.1 uORF ′5 STOP: 68 uORF 3′ STOP: 152 leader length: 290 |
| 2975 | AGI number: AT4G26520.1 uORF ′5 STOP: 82 uORF 3′ STOP: 133 leader length: 290 |
| 2976 | AGI number: AT4G26520.1 uORF ′5 STOP: 82 uORF 3′ STOP: 133 leader length: 290 |
| 2977 | AGI number: AT4G26650.1 uORF ′5 STOP: 21 uORF 3′ STOP: 75 leader length: 521 |
| 2978 | AGI number: AT4G26650.1 uORF ′5 STOP: 21 uORF 3′ STOP: 75 leader length: 521 |
| 2979 | AGI number: AT4G26850.1 uORF ′5 STOP: 141 uORF 3′ STOP: 393 leader length: 503 |
| 2980 | AGI number: AT4G26850.1 uORF ′5 STOP: 141 uORF 3′ STOP: 393 leader length: 503 |
| 2981 | AGI number: AT4G26850.1 uORF ′5 STOP: 265 uORF 3′ STOP: 400 leader length: 503 |
| 2982 | AGI number: AT4G26850.1 uORF ′5 STOP: 265 uORF 3′ STOP: 400 leader length: 503 |
| 2983 | AGI number: AT4G26850.1 uORF ′5 STOP: 314 uORF 3′ STOP: 371 leader length: 503 |
| 2984 | AGI number: AT4G26850.1 uORF ′5 STOP: 314 uORF 3′ STOP: 371 leader length: 503 |
| 2985 | AGI number: AT4G27010.2 uORF ′5 STOP: 347 uORF 3′ STOP: 419 leader length: 671 |
| 2986 | AGI number: AT4G27010.2 uORF ′5 STOP: 347 uORF 3′ STOP: 419 leader length: 671 |
| 2987 | AGI number: AT4G27040.1 uORF ′5 STOP: 336 uORF 3′ STOP: 435 leader length: 555 |
| 2988 | AGI number: AT4G27040.1 uORF ′5 STOP: 336 uORF 3′ STOP: 435 leader length: 555 |
| 2989 | AGI number: AT4G27040.2 uORF ′5 STOP: 79 uORF 3′ STOP: 157 leader length: 689 |
| 2990 | AGI number: AT4G27040.2 uORF ′5 STOP: 79 uORF 3′ STOP: 157 leader length: 689 |
| 2991 | AGI number: AT4G27040.2 uORF ′5 STOP: 105 uORF 3′ STOP: 168 leader length: 689 |
| 2992 | AGI number: AT4G27040.2 uORF ′5 STOP: 105 uORF 3′ STOP: 168 leader length: 689 |
| 2993 | AGI number: AT4G27040.2 uORF ′5 STOP: 154 uORF 3′ STOP: 205 leader length: 689 |
| 2994 | AGI number: AT4G27040.2 uORF ′5 STOP: 154 uORF 3′ STOP: 205 leader length: 689 |
| 2995 | AGI number: AT4G27040.4 uORF ′5 STOP: 222 uORF 3′ STOP: 279 leader length: 504 |
| 2996 | AGI number: AT4G27040.4 uORF ′5 STOP: 222 uORF 3′ STOP: 279 leader length: 504 |
| 2997 | AGI number: AT4G27050.1 uORF ′5 STOP: 93 uORF 3′ STOP: 258 leader length: 453 |
| 2998 | AGI number: AT4G27050.1 uORF ′5 STOP: 93 uORF 3′ STOP: 258 leader length: 453 |
| 2999 | AGI number: AT4G27050.1 uORF ′5 STOP: 97 uORF 3′ STOP: 151 leader length: 453 |
| 3000 | AGI number: AT4G27050.1 uORF ′5 STOP: 97 uORF 3′ STOP: 151 leader length: 453 |
| 3001 | AGI number: AT4G27050.1 uORF ′5 STOP: 181 uORF 3′ STOP: 268 leader length: 453 |
| 3002 | AGI number: AT4G27050.1 uORF ′5 STOP: 181 uORF 3′ STOP: 268 leader length: 453 |
| 3003 | AGI number: AT4G27050.2 uORF ′5 STOP: 35 uORF 3′ STOP: 200 leader length: 477 |
| 3004 | AGI number: AT4G27050.2 uORF ′5 STOP: 35 uORF 3′ STOP: 200 leader length: 477 |
| 3005 | AGI number: AT4G27050.2 uORF ′5 STOP: 123 uORF 3′ STOP: 210 leader length: 477 |
| 3006 | AGI number: AT4G27050.2 uORF ′5 STOP: 123 uORF 3′ STOP: 210 leader length: 477 |
| 3007 | AGI number: AT4G27050.2 uORF ′5 STOP: 331 uORF 3′ STOP: 406 leader length: 477 |
| 3008 | AGI number: AT4G27050.2 uORF ′5 STOP: 331 uORF 3′ STOP: 406 leader length: 477 |
| 3009 | AGI number: AT4G27050.2 uORF ′5 STOP: 339 uORF 3′ STOP: 420 leader length: 477 |
| 3010 | AGI number: AT4G27050.2 uORF ′5 STOP: 339 uORF 3′ STOP: 420 leader length: 477 |
| 3011 | AGI number: AT4G27050.3 uORF ′5 STOP: 93 uORF 3′ STOP: 219 leader length: 290 |
| 3012 | AGI number: AT4G27050.3 uORF ′5 STOP: 93 uORF 3′ STOP: 219 leader length: 290 |
| 3013 | AGI number: AT4G27080.2 uORF ′5 STOP: 65 uORF 3′ STOP: 143 leader length: 84 |
| 3014 | AGI number: AT4G27080.2 uORF ′5 STOP: 65 uORF 3′ STOP: 143 leader length: 84 |
| 3015 | AGI number: AT4G27080.2 uORF ′5 STOP: 39 uORF 3′ STOP: 117 leader length: 84 |
| 3016 | AGI number: AT4G27080.2 uORF ′5 STOP: 39 uORF 3′ STOP: 117 leader length: 84 |
| 3017 | AGI number: AT4G27610.3 uORF ′5 STOP: 334 uORF 3′ STOP: 451 leader length: 1078 |
| 3018 | AGI number: AT4G27610.3 uORF ′5 STOP: 334 uORF 3′ STOP: 451 leader length: 1078 |
| 3019 | AGI number: AT4G27610.3 uORF ′5 STOP: 383 uORF 3′ STOP: 473 leader length: 1078 |
| 3020 | AGI number: AT4G27610.3 uORF ′5 STOP: 383 uORF 3′ STOP: 473 leader length: 1078 |
| 3021 | AGI number: AT4G27610.3 uORF ′5 STOP: 429 uORF 3′ STOP: 495 leader length: 1078 |
| 3022 | AGI number: AT4G27610.3 uORF ′5 STOP: 429 uORF 3′ STOP: 495 leader length: 1078 |
| 3023 | AGI number: AT4G27840.1 uORF ′5 STOP: 24 uORF 3′ STOP: 87 leader length: 237 |
| 3024 | AGI number: AT4G27840.1 uORF ′5 STOP: 24 uORF 3′ STOP: 87 leader length: 237 |
| 3025 | AGI number: AT4G28040.4 uORF ′5 STOP: 63 uORF 3′ STOP: 165 leader length: 288 |
| 3026 | AGI number: AT4G28040.4 uORF ′5 STOP: 63 uORF 3′ STOP: 165 leader length: 288 |
| 3027 | AGI number: AT4G28260.1 uORF ′5 STOP: 21 uORF 3′ STOP: 315 leader length: 563 |
| 3028 | AGI number: AT4G28260.1 uORF ′5 STOP: 21 uORF 3′ STOP: 315 leader length: 563 |
| 3029 | AGI number: AT4G28260.1 uORF ′5 STOP: 227 uORF 3′ STOP: 305 leader length: 563 |
| 3030 | AGI number: AT4G28260.1 uORF ′5 STOP: 227 uORF 3′ STOP: 305 leader length: 563 |
| 3031 | AGI number: AT4G28260.2 uORF ′5 STOP: 21 uORF 3′ STOP: 315 leader length: 474 |
| 3032 | AGI number: AT4G28260.2 uORF ′5 STOP: 21 uORF 3′ STOP: 315 leader length: 474 |
| 3033 | AGI number: AT4G28260.2 uORF ′5 STOP: 227 uORF 3′ STOP: 305 leader length: 474 |
| 3034 | AGI number: AT4G28260.2 uORF ′5 STOP: 227 uORF 3′ STOP: 305 leader length: 474 |
| 3035 | AGI number: AT4G28300.2 uORF ′5 STOP: 119 uORF 3′ STOP: 383 leader length: 741 |
| 3036 | AGI number: AT4G28300.2 uORF ′5 STOP: 119 uORF 3′ STOP: 383 leader length: 741 |
| 3037 | AGI number: AT4G28300.2 uORF ′5 STOP: 295 uORF 3′ STOP: 349 leader length: 741 |
| 3038 | AGI number: AT4G28300.2 uORF ′5 STOP: 295 uORF 3′ STOP: 349 leader length: 741 |
| 3039 | AGI number: AT4G28480.1 uORF ′5 STOP: 6 uORF 3′ STOP: 69 leader length: 268 |
| 3040 | AGI number: AT4G28480.1 uORF ′5 STOP: 6 uORF 3′ STOP: 69 leader length: 268 |
| 3041 | AGI number: AT4G28480.2 uORF ′5 STOP: 6 uORF 3′ STOP: 69 leader length: 268 |
| 3042 | AGI number: AT4G28480.2 uORF ′5 STOP: 6 uORF 3′ STOP: 69 leader length: 268 |
| 3043 | AGI number: AT4G29160.2 uORF ′5 STOP: 220 uORF 3′ STOP: 274 leader length: 546 |
| 3044 | AGI number: AT4G29160.2 uORF ′5 STOP: 220 uORF 3′ STOP: 274 leader length: 546 |
| 3045 | AGI number: AT4G29160.2 uORF ′5 STOP: 188 uORF 3′ STOP: 257 leader length: 546 |
| 3046 | AGI number: AT4G29160.2 uORF ′5 STOP: 188 uORF 3′ STOP: 257 leader length: 546 |
| 3047 | AGI number: AT4G30020.1 uORF ′5 STOP: 57 uORF 3′ STOP: 210 leader length: 315 |
| 3048 | AGI number: AT4G30020.1 uORF ′5 STOP: 57 uORF 3′ STOP: 210 leader length: 315 |
| 3049 | AGI number: AT4G30160.1 uORF ′5 STOP: 109 uORF 3′ STOP: 160 leader length: 486 |
| 3050 | AGI number: AT4G30160.1 uORF ′5 STOP: 109 uORF 3′ STOP: 160 leader length: 486 |
| 3051 | AGI number: AT4G30630.1 uORF ′5 STOP: 94 uORF 3′ STOP: 283 leader length: 410 |
| 3052 | AGI number: AT4G30630.1 uORF ′5 STOP: 94 uORF 3′ STOP: 283 leader length: 410 |
| 3053 | AGI number: AT4G30710.1 uORF ′5 STOP: 355 uORF 3′ STOP: 409 leader length: 652 |
| 3054 | AGI number: AT4G30710.1 uORF ′5 STOP: 355 uORF 3′ STOP: 409 leader length: 652 |
| 3055 | AGI number: AT4G30900.1 uORF ′5 STOP: 109 uORF 3′ STOP: 163 leader length: 386 |
| 3056 | AGI number: AT4G30900.1 uORF ′5 STOP: 109 uORF 3′ STOP: 163 leader length: 386 |
| 3057 | AGI number: AT4G30900.2 uORF ′5 STOP: 86 uORF 3′ STOP: 137 leader length: 461 |
| 3058 | AGI number: AT4G30900.2 uORF ′5 STOP: 86 uORF 3′ STOP: 137 leader length: 461 |
| 3059 | AGI number: AT4G30900.2 uORF ′5 STOP: 235 uORF 3′ STOP: 370 leader length: 461 |
| 3060 | AGI number: AT4G30900.2 uORF ′5 STOP: 235 uORF 3′ STOP: 370 leader length: 461 |
| 3061 | AGI number: AT4G30960.1 uORF ′5 STOP: 111 uORF 3′ STOP: 231 leader length: 347 |
| 3062 | AGI number: AT4G30960.1 uORF ′5 STOP: 111 uORF 3′ STOP: 231 leader length: 347 |
| 3063 | AGI number: AT4G30960.1 uORF ′5 STOP: 151 uORF 3′ STOP: 244 leader length: 347 |
| 3064 | AGI number: AT4G30960.1 uORF ′5 STOP: 151 uORF 3′ STOP: 244 leader length: 347 |
| 3065 | AGI number: AT4G31820.1 uORF ′5 STOP: 362 uORF 3′ STOP: 485 leader length: 803 |
| 3066 | AGI number: AT4G31820.1 uORF ′5 STOP: 362 uORF 3′ STOP: 485 leader length: 803 |
| 3067 | AGI number: AT4G31880.1 uORF ′5 STOP: 83 uORF 3′ STOP: 149 leader length: 244 |
| 3068 | AGI number: AT4G31880.1 uORF ′5 STOP: 83 uORF 3′ STOP: 149 leader length: 244 |
| 3069 | AGI number: AT4G31880.2 uORF ′5 STOP: 43 uORF 3′ STOP: 217 leader length: 326 |
| 3070 | AGI number: AT4G31880.2 uORF ′5 STOP: 43 uORF 3′ STOP: 217 leader length: 326 |
| 3071 | AGI number: AT4G31880.2 uORF ′5 STOP: 165 uORF 3′ STOP: 231 leader length: 326 |
| 3072 | AGI number: AT4G31880.2 uORF ′5 STOP: 165 uORF 3′ STOP: 231 leader length: 326 |
| 3073 | AGI number: AT4G31990.1 uORF ′5 STOP: 2 uORF 3′ STOP: 68 leader length: 227 |
| 3074 | AGI number: AT4G31990.1 uORF ′5 STOP: 2 uORF 3′ STOP: 68 leader length: 227 |
| 3075 | AGI number: AT4G31990.1 uORF ′5 STOP: 13 uORF 3′ STOP: 82 leader length: 227 |
| 3076 | AGI number: AT4G31990.1 uORF ′5 STOP: 13 uORF 3′ STOP: 82 leader length: 227 |
| 3077 | AGI number: AT4G32300.1 uORF ′5 STOP: 94 uORF 3′ STOP: 145 leader length: 495 |
| 3078 | AGI number: AT4G32300.1 uORF ′5 STOP: 94 uORF 3′ STOP: 145 leader length: 495 |
| 3079 | AGI number: AT4G32850.3 uORF ′5 STOP: 52 uORF 3′ STOP: 109 leader length: 354 |
| 3080 | AGI number: AT4G32850.3 uORF ′5 STOP: 52 uORF 3′ STOP: 109 leader length: 354 |
| 3081 | AGI number: AT4G32980.1 uORF ′5 STOP: 190 uORF 3′ STOP: 316 leader length: 703 |
| 3082 | AGI number: AT4G32980.1 uORF ′5 STOP: 190 uORF 3′ STOP: 316 leader length: 703 |
| 3083 | AGI number: AT4G32980.1 uORF ′5 STOP: 527 uORF 3′ STOP: 620 leader length: 703 |
| 3084 | AGI number: AT4G32980.1 uORF ′5 STOP: 527 uORF 3′ STOP: 620 leader length: 703 |
| 3085 | AGI number: AT4G33050.2 uORF ′5 STOP: 125 uORF 3′ STOP: 206 leader length: 194 |
| 3086 | AGI number: AT4G33050.2 uORF ′5 STOP: 125 uORF 3′ STOP: 206 leader length: 194 |
| 3087 | AGI number: AT4G33160.1 uORF ′5 STOP: 26 uORF 3′ STOP: 92 leader length: 71 |
| 3088 | AGI number: AT4G33160.1 uORF ′5 STOP: 26 uORF 3′ STOP: 92 leader length: 71 |
| 3089 | AGI number: AT4G33240.1 uORF ′5 STOP: 207 uORF 3′ STOP: 261 leader length: 260 |
| 3090 | AGI number: AT4G33240.1 uORF ′5 STOP: 207 uORF 3′ STOP: 261 leader length: 260 |
| 3091 | AGI number: AT4G33950.2 uORF ′5 STOP: 251 uORF 3′ STOP: 566 leader length: 604 |
| 3092 | AGI number: AT4G33950.2 uORF ′5 STOP: 251 uORF 3′ STOP: 566 leader length: 604 |
| 3093 | AGI number: AT4G34160.1 uORF ′5 STOP: 83 uORF 3′ STOP: 176 leader length: 264 |
| 3094 | AGI number: AT4G34160.1 uORF ′5 STOP: 83 uORF 3′ STOP: 176 leader length: 264 |
| 3095 | AGI number: AT4G34160.1 uORF ′5 STOP: 102 uORF 3′ STOP: 168 leader length: 264 |
| 3096 | AGI number: AT4G34160.1 uORF ′5 STOP: 102 uORF 3′ STOP: 168 leader length: 264 |
| 3097 | AGI number: AT4G34420.1 uORF ′5 STOP: 58 uORF 3′ STOP: 139 leader length: 362 |
| 3098 | AGI number: AT4G34420.1 uORF ′5 STOP: 58 uORF 3′ STOP: 139 leader length: 362 |
| 3099 | AGI number: AT4G34460.2 uORF ′5 STOP: 381 uORF 3′ STOP: 444 leader length: 663 |
| 3100 | AGI number: AT4G34460.2 uORF ′5 STOP: 381 uORF 3′ STOP: 444 leader length: 663 |
| 3101 | AGI number: AT4G34540.1 uORF ′5 STOP: 983 uORF 3′ STOP: 1076 leader length: 1642 |
| 3102 | AGI number: AT4G34540.1 uORF ′5 STOP: 983 uORF 3′ STOP: 1076 leader length: 1642 |
| 3103 | AGI number: AT4G34560.1 uORF ′5 STOP: 140 uORF 3′ STOP: 212 leader length: 288 |
| 3104 | AGI number: AT4G34560.1 uORF ′5 STOP: 140 uORF 3′ STOP: 212 leader length: 288 |
| 3105 | AGI number: AT4G34590.1 uORF ′5 STOP: 202 uORF 3′ STOP: 367 leader length: 588 |
| 3106 | AGI number: AT4G34590.1 uORF ′5 STOP: 202 uORF 3′ STOP: 367 leader length: 588 |
| 3107 | AGI number: AT4G34590.1 uORF ′5 STOP: 284 uORF 3′ STOP: 374 leader length: 588 |
| 3108 | AGI number: AT4G34590.1 uORF ′5 STOP: 284 uORF 3′ STOP: 374 leader length: 588 |
| 3109 | AGI number: AT4G34710.1 uORF ′5 STOP: 43 uORF 3′ STOP: 331 leader length: 519 |
| 3110 | AGI number: AT4G34710.1 uORF ′5 STOP: 43 uORF 3′ STOP: 331 leader length: 519 |
| 3111 | AGI number: AT4G34710.1 uORF ′5 STOP: 177 uORF 3′ STOP: 339 leader length: 519 |
| 3112 | AGI number: AT4G34710.1 uORF ′5 STOP: 177 uORF 3′ STOP: 339 leader length: 519 |
| 3113 | AGI number: AT4G34710.2 uORF ′5 STOP: 5 uORF 3′ STOP: 293 leader length: 384 |
| 3114 | AGI number: AT4G34710.2 uORF ′5 STOP: 5 uORF 3′ STOP: 293 leader length: 384 |
| 3115 | AGI number: AT4G34710.2 uORF ′5 STOP: 139 uORF 3′ STOP: 301 leader length: 384 |
| 3116 | AGI number: AT4G34710.2 uORF ′5 STOP: 139 uORF 3′ STOP: 301 leader length: 384 |
| 3117 | AGI number: AT4G34881.1 uORF ′5 STOP: 104 uORF 3′ STOP: 236 leader length: 269 |
| 3118 | AGI number: AT4G34881.1 uORF ′5 STOP: 104 uORF 3′ STOP: 236 leader length: 269 |
| 3119 | AGI number: AT4G35020.2 uORF ′5 STOP: 267 uORF 3′ STOP: 354 leader length: 517 |
| 3120 | AGI number: AT4G35020.2 uORF ′5 STOP: 267 uORF 3′ STOP: 354 leader length: 517 |
| 3121 | AGI number: AT4G35020.3 uORF ′5 STOP: 47 uORF 3′ STOP: 122 leader length: 285 |
| 3122 | AGI number: AT4G35020.3 uORF ′5 STOP: 47 uORF 3′ STOP: 122 leader length: 285 |
| 3123 | AGI number: AT4G35040.1 uORF ′5 STOP: 80 uORF 3′ STOP: 131 leader length: 400 |
| 3124 | AGI number: AT4G35040.1 uORF ′5 STOP: 80 uORF 3′ STOP: 131 leader length: 400 |
| 3125 | AGI number: AT4G35390.1 uORF ′5 STOP: 15 uORF 3′ STOP: 90 leader length: 277 |
| 3126 | AGI number: AT4G35390.1 uORF ′5 STOP: 15 uORF 3′ STOP: 90 leader length: 277 |
| 3127 | AGI number: AT4G35470.1 uORF ′5 STOP: 33 uORF 3′ STOP: 129 leader length: 230 |
| 3128 | AGI number: AT4G35470.1 uORF ′5 STOP: 33 uORF 3′ STOP: 129 leader length: 230 |
| 3129 | AGI number: AT4G35860.2 uORF ′5 STOP: 22 uORF 3′ STOP: 259 leader length: 460 |
| 3130 | AGI number: AT4G35860.2 uORF ′5 STOP: 22 uORF 3′ STOP: 259 leader length: 460 |
| 3131 | AGI number: AT4G35860.2 uORF ′5 STOP: 62 uORF 3′ STOP: 284 leader length: 460 |
| 3132 | AGI number: AT4G35860.2 uORF ′5 STOP: 62 uORF 3′ STOP: 284 leader length: 460 |
| 3133 | AGI number: AT4G35860.2 uORF ′5 STOP: 174 uORF 3′ STOP: 240 leader length: 460 |
| 3134 | AGI number: AT4G35860.2 uORF ′5 STOP: 174 uORF 3′ STOP: 240 leader length: 460 |
| 3135 | AGI number: AT4G35920.2 uORF ′5 STOP: 18 uORF 3′ STOP: 69 leader length: 350 |
| 3136 | AGI number: AT4G35920.2 uORF ′5 STOP: 18 uORF 3′ STOP: 69 leader length: 350 |
| 3137 | AGI number: AT4G35940.2 uORF ′5 STOP: 97 uORF 3′ STOP: 160 leader length: 100 |
| 3138 | AGI number: AT4G35940.2 uORF ′5 STOP: 97 uORF 3′ STOP: 160 leader length: 100 |
| 3139 | AGI number: AT4G36050.1 uORF ′5 STOP: 523 uORF 3′ STOP: 577 leader length: 699 |
| 3140 | AGI number: AT4G36050.1 uORF ′5 STOP: 523 uORF 3′ STOP: 577 leader length: 699 |
| 3141 | AGI number: AT4G36050.1 uORF ′5 STOP: 527 uORF 3′ STOP: 581 leader length: 699 |
| 3142 | AGI number: AT4G36050.1 uORF ′5 STOP: 527 uORF 3′ STOP: 581 leader length: 699 |
| 3143 | AGI number: AT4G36090.2 uORF ′5 STOP: 115 uORF 3′ STOP: 274 leader length: 601 |
| 3144 | AGI number: AT4G36090.2 uORF ′5 STOP: 115 uORF 3′ STOP: 274 leader length: 601 |
| 3145 | AGI number: AT4G36620.1 uORF ′5 STOP: 80 uORF 3′ STOP: 242 leader length: 81 |
| 3146 | AGI number: AT4G36620.1 uORF ′5 STOP: 80 uORF 3′ STOP: 242 leader length: 81 |
| 3147 | AGI number: AT4G36840.1 uORF ′5 STOP: 648 uORF 3′ STOP: 705 leader length: 813 |
| 3148 | AGI number: AT4G36840.1 uORF ′5 STOP: 648 uORF 3′ STOP: 705 leader length: 813 |
| 3149 | AGI number: AT4G36960.2 uORF ′5 STOP: 65 uORF 3′ STOP: 227 leader length: 366 |
| 3150 | AGI number: AT4G36960.2 uORF ′5 STOP: 65 uORF 3′ STOP: 227 leader length: 366 |
| 3151 | AGI number: AT4G37080.3 uORF ′5 STOP: 73 uORF 3′ STOP: 139 leader length: 407 |
| 3152 | AGI number: AT4G37080.3 uORF ′5 STOP: 73 uORF 3′ STOP: 139 leader length: 407 |
| 3153 | AGI number: AT4G37590.1 uORF ′5 STOP: 704 uORF 3′ STOP: 773 leader length: 870 |
| 3154 | AGI number: AT4G37590.1 uORF ′5 STOP: 704 uORF 3′ STOP: 773 leader length: 870 |
| 3155 | AGI number: AT4G37608.1 uORF ′5 STOP: 25 uORF 3′ STOP: 133 leader length: 199 |
| 3156 | AGI number: AT4G37608.1 uORF ′5 STOP: 25 uORF 3′ STOP: 133 leader length: 199 |
| 3157 | AGI number: AT4G37690.1 uORF ′5 STOP: 358 uORF 3′ STOP: 694 leader length: 409 |
| 3158 | AGI number: AT4G37690.1 uORF ′5 STOP: 358 uORF 3′ STOP: 694 leader length: 409 |
| 3159 | AGI number: AT4G37750.1 uORF ′5 STOP: 54 uORF 3′ STOP: 168 leader length: 268 |
| 3160 | AGI number: AT4G37750.1 uORF ′5 STOP: 54 uORF 3′ STOP: 168 leader length: 268 |
| 3161 | AGI number: AT4G38260.1 uORF ′5 STOP: 13 uORF 3′ STOP: 97 leader length: 518 |
| 3162 | AGI number: AT4G38260.1 uORF ′5 STOP: 13 uORF 3′ STOP: 97 leader length: 518 |
| 3163 | AGI number: AT4G38260.1 uORF ′5 STOP: 214 uORF 3′ STOP: 307 leader length: 518 |
| 3164 | AGI number: AT4G38260.1 uORF ′5 STOP: 214 uORF 3′ STOP: 307 leader length: 518 |
| 3165 | AGI number: AT4G38330.1 uORF ′5 STOP: 180 uORF 3′ STOP: 264 leader length: 269 |
| 3166 | AGI number: AT4G38330.1 uORF ′5 STOP: 180 uORF 3′ STOP: 264 leader length: 269 |
| 3167 | AGI number: AT4G38520.1 uORF ′5 STOP: 368 uORF 3′ STOP: 668 leader length: 527 |
| 3168 | AGI number: AT4G38520.1 uORF ′5 STOP: 368 uORF 3′ STOP: 668 leader length: 527 |
| 3169 | AGI number: AT4G38520.2 uORF ′5 STOP: 198 uORF 3′ STOP: 351 leader length: 759 |
| 3170 | AGI number: AT4G38520.2 uORF ′5 STOP: 198 uORF 3′ STOP: 351 leader length: 759 |
| 3171 | AGI number: AT4G38520.2 uORF ′5 STOP: 181 uORF 3′ STOP: 331 leader length: 759 |
| 3172 | AGI number: AT4G38520.2 uORF ′5 STOP: 181 uORF 3′ STOP: 331 leader length: 759 |
| 3173 | AGI number: AT4G38520.2 uORF ′5 STOP: 600 uORF 3′ STOP: 900 leader length: 759 |
| 3174 | AGI number: AT4G38520.2 uORF ′5 STOP: 600 uORF 3′ STOP: 900 leader length: 759 |
| 3175 | AGI number: AT4G38950.1 uORF ′5 STOP: 533 uORF 3′ STOP: 674 leader length: 581 |
| 3176 | AGI number: AT4G38950.1 uORF ′5 STOP: 533 uORF 3′ STOP: 674 leader length: 581 |
| 3177 | AGI number: AT4G38960.3 uORF ′5 STOP: 401 uORF 3′ STOP: 551 leader length: 488 |
| 3178 | AGI number: AT4G38960.3 uORF ′5 STOP: 401 uORF 3′ STOP: 551 leader length: 488 |
| 3179 | AGI number: AT4G39050.1 uORF ′5 STOP: 57 uORF 3′ STOP: 234 leader length: 327 |
| 3180 | AGI number: AT4G39050.1 uORF ′5 STOP: 57 uORF 3′ STOP: 234 leader length: 327 |
| 3181 | AGI number: AT4G39180.1 uORF ′5 STOP: 169 uORF 3′ STOP: 331 leader length: 424 |
| 3182 | AGI number: AT4G39180.1 uORF ′5 STOP: 169 uORF 3′ STOP: 331 leader length: 424 |
| 3183 | AGI number: AT4G39180.1 uORF ′5 STOP: 177 uORF 3′ STOP: 390 leader length: 424 |
| 3184 | AGI number: AT4G39180.1 uORF ′5 STOP: 177 uORF 3′ STOP: 390 leader length: 424 |
| 3185 | AGI number: AT4G39190.1 uORF ′5 STOP: 41 uORF 3′ STOP: 104 leader length: 208 |
| 3186 | AGI number: AT4G39190.1 uORF ′5 STOP: 41 uORF 3′ STOP: 104 leader length: 208 |
| 3187 | AGI number: AT4G39270.2 uORF ′5 STOP: 29 uORF 3′ STOP: 101 leader length: 369 |
| 3188 | AGI number: AT4G39270.2 uORF ′5 STOP: 29 uORF 3′ STOP: 101 leader length: 369 |
| 3189 | AGI number: AT4G39390.1 uORF ′5 STOP: 132 uORF 3′ STOP: 192 leader length: 301 |
| 3190 | AGI number: AT4G39390.1 uORF ′5 STOP: 132 uORF 3′ STOP: 192 leader length: 301 |
| 3191 | AGI number: AT4G39390.1 uORF ′5 STOP: 124 uORF 3′ STOP: 181 leader length: 301 |
| 3192 | AGI number: AT4G39390.1 uORF ′5 STOP: 124 uORF 3′ STOP: 181 leader length: 301 |
| 3193 | AGI number: AT4G39390.2 uORF ′5 STOP: 68 uORF 3′ STOP: 125 leader length: 223 |
| 3194 | AGI number: AT4G39390.2 uORF ′5 STOP: 68 uORF 3′ STOP: 125 leader length: 223 |
| 3195 | AGI number: AT4G39390.2 uORF ′5 STOP: 76 uORF 3′ STOP: 136 leader length: 223 |
| 3196 | AGI number: AT4G39390.2 uORF ′5 STOP: 76 uORF 3′ STOP: 136 leader length: 223 |
| 3197 | AGI number: AT4G39390.3 uORF ′5 STOP: 102 uORF 3′ STOP: 162 leader length: 356 |
| 3198 | AGI number: AT4G39390.3 uORF ′5 STOP: 102 uORF 3′ STOP: 162 leader length: 356 |
| 3199 | AGI number: AT4G39390.3 uORF ′5 STOP: 94 uORF 3′ STOP: 151 leader length: 356 |
| 3200 | AGI number: AT4G39390.3 uORF ′5 STOP: 94 uORF 3′ STOP: 151 leader length: 356 |
| 3201 | AGI number: AT4G39420.1 uORF ′5 STOP: 19 uORF 3′ STOP: 145 leader length: 242 |
| 3202 | AGI number: AT4G39420.1 uORF ′5 STOP: 19 uORF 3′ STOP: 145 leader length: 242 |
| 3203 | AGI number: AT4G39420.2 uORF ′5 STOP: 19 uORF 3′ STOP: 145 leader length: 242 |
| 3204 | AGI number: AT4G39420.2 uORF ′5 STOP: 19 uORF 3′ STOP: 145 leader length: 242 |
| 3205 | AGI number: AT4G39680.1 uORF ′5 STOP: 32 uORF 3′ STOP: 125 leader length: 266 |
| 3206 | AGI number: AT4G39680.1 uORF ′5 STOP: 32 uORF 3′ STOP: 125 leader length: 266 |
| 3207 | AGI number: AT4G39680.2 uORF ′5 STOP: 32 uORF 3′ STOP: 125 leader length: 207 |
| 3208 | AGI number: AT4G39680.2 uORF ′5 STOP: 32 uORF 3′ STOP: 125 leader length: 207 |
| 3209 | AGI number: AT4G40030.3 uORF ′5 STOP: 30 uORF 3′ STOP: 84 leader length: 372 |
| 3210 | AGI number: AT4G40030.3 uORF ′5 STOP: 30 uORF 3′ STOP: 84 leader length: 372 |
| 3211 | AGI number: AT4G40030.3 uORF ′5 STOP: 171 uORF 3′ STOP: 246 leader length: 372 |
| 3212 | AGI number: AT4G40030.3 uORF ′5 STOP: 171 uORF 3′ STOP: 246 leader length: 372 |
| 3213 | AGI number: AT4G40040.2 uORF ′5 STOP: 23 uORF 3′ STOP: 86 leader length: 262 |
| 3214 | AGI number: AT4G40040.2 uORF ′5 STOP: 23 uORF 3′ STOP: 86 leader length: 262 |
| 3215 | AGI number: AT4G40040.2 uORF ′5 STOP: 30 uORF 3′ STOP: 123 leader length: 262 |
| 3216 | AGI number: AT4G40040.2 uORF ′5 STOP: 30 uORF 3′ STOP: 123 leader length: 262 |
| 3217 | AGI number: AT5G01100.1 uORF ′5 STOP: 22 uORF 3′ STOP: 145 leader length: 192 |
| 3218 | AGI number: AT5G01100.1 uORF ′5 STOP: 22 uORF 3′ STOP: 145 leader length: 192 |
| 3219 | AGI number: AT5G01100.1 uORF ′5 STOP: 129 uORF 3′ STOP: 279 leader length: 192 |
| 3220 | AGI number: AT5G01100.1 uORF ′5 STOP: 129 uORF 3′ STOP: 279 leader length: 192 |
| 3221 | AGI number: AT5G01300.2 uORF ′5 STOP: 68 uORF 3′ STOP: 152 leader length: 379 |
| 3222 | AGI number: AT5G01300.2 uORF ′5 STOP: 68 uORF 3′ STOP: 152 leader length: 379 |
| 3223 | AGI number: AT5G01380.1 uORF ′5 STOP: 149 uORF 3′ STOP: 212 leader length: 150 |
| 3224 | AGI number: AT5G01380.1 uORF ′5 STOP: 149 uORF 3′ STOP: 212 leader length: 150 |
| 3225 | AGI number: AT5G01520.1 uORF ′5 STOP: 103 uORF 3′ STOP: 235 leader length: 365 |
| 3226 | AGI number: AT5G01520.1 uORF ′5 STOP: 103 uORF 3′ STOP: 235 leader length: 365 |
| 3227 | AGI number: AT5G01520.1 uORF ′5 STOP: 167 uORF 3′ STOP: 254 leader length: 365 |
| 3228 | AGI number: AT5G01520.1 uORF ′5 STOP: 167 uORF 3′ STOP: 254 leader length: 365 |
| 3229 | AGI number: AT5G01520.2 uORF ′5 STOP: 103 uORF 3′ STOP: 235 leader length: 365 |
| 3230 | AGI number: AT5G01520.2 uORF ′5 STOP: 103 uORF 3′ STOP: 235 leader length: 365 |
| 3231 | AGI number: AT5G01520.2 uORF ′5 STOP: 167 uORF 3′ STOP: 254 leader length: 365 |
| 3232 | AGI number: AT5G01520.2 uORF ′5 STOP: 167 uORF 3′ STOP: 254 leader length: 365 |
| 3233 | AGI number: AT5G01710.1 uORF ′5 STOP: 222 uORF 3′ STOP: 285 leader length: 383 |
| 3234 | AGI number: AT5G01710.1 uORF ′5 STOP: 222 uORF 3′ STOP: 285 leader length: 383 |
| 3235 | AGI number: AT5G01810.1 uORF ′5 STOP: 100 uORF 3′ STOP: 340 leader length: 464 |
| 3236 | AGI number: AT5G01810.1 uORF ′5 STOP: 100 uORF 3′ STOP: 340 leader length: 464 |
| 3237 | AGI number: AT5G01810.1 uORF ′5 STOP: 251 uORF 3′ STOP: 365 leader length: 464 |
| 3238 | AGI number: AT5G01810.1 uORF ′5 STOP: 251 uORF 3′ STOP: 365 leader length: 464 |
| 3239 | AGI number: AT5G01810.2 uORF ′5 STOP: 212 uORF 3′ STOP: 452 leader length: 576 |
| 3240 | AGI number: AT5G01810.2 uORF ′5 STOP: 212 uORF 3′ STOP: 452 leader length: 576 |
| 3241 | AGI number: AT5G01810.2 uORF ′5 STOP: 363 uORF 3′ STOP: 477 leader length: 576 |
| 3242 | AGI number: AT5G01810.2 uORF ′5 STOP: 363 uORF 3′ STOP: 477 leader length: 576 |
| 3243 | AGI number: AT5G01810.3 uORF ′5 STOP: 212 uORF 3′ STOP: 452 leader length: 576 |
| 3244 | AGI number: AT5G01810.3 uORF ′5 STOP: 212 uORF 3′ STOP: 452 leader length: 576 |
| 3245 | AGI number: AT5G01810.3 uORF ′5 STOP: 363 uORF 3′ STOP: 477 leader length: 576 |
| 3246 | AGI number: AT5G01810.3 uORF ′5 STOP: 363 uORF 3′ STOP: 477 leader length: 576 |
| 3247 | AGI number: AT5G01830.1 uORF ′5 STOP: 133 uORF 3′ STOP: 187 leader length: 288 |
| 3248 | AGI number: AT5G01830.1 uORF ′5 STOP: 133 uORF 3′ STOP: 187 leader length: 288 |
| 3249 | AGI number: AT5G02060.1 uORF ′5 STOP: 82 uORF 3′ STOP: 235 leader length: 344 |
| 3250 | AGI number: AT5G02060.1 uORF ′5 STOP: 82 uORF 3′ STOP: 235 leader length: 344 |
| 3251 | AGI number: AT5G02480.1 uORF ′5 STOP: 263 uORF 3′ STOP: 404 leader length: 589 |
| 3252 | AGI number: AT5G02480.1 uORF ′5 STOP: 263 uORF 3′ STOP: 404 leader length: 589 |
| 3253 | AGI number: AT5G02550.1 uORF ′5 STOP: 77 uORF 3′ STOP: 341 leader length: 81 |
| 3254 | AGI number: AT5G02550.1 uORF ′5 STOP: 77 uORF 3′ STOP: 341 leader length: 81 |
| 3255 | AGI number: AT5G02650.1 uORF ′5 STOP: 244 uORF 3′ STOP: 367 leader length: 307 |
| 3256 | AGI number: AT5G02650.1 uORF ′5 STOP: 244 uORF 3′ STOP: 367 leader length: 307 |
| 3257 | AGI number: AT5G02680.1 uORF ′5 STOP: 16 uORF 3′ STOP: 190 leader length: 173 |
| 3258 | AGI number: AT5G02680.1 uORF ′5 STOP: 16 uORF 3′ STOP: 190 leader length: 173 |
| 3259 | AGI number: AT5G03190.1 uORF ′5 STOP: 251 uORF 3′ STOP: 344 leader length: 419 |
| 3260 | AGI number: AT5G03190.1 uORF ′5 STOP: 251 uORF 3′ STOP: 344 leader length: 419 |
| 3261 | AGI number: AT5G03190.1 uORF ′5 STOP: 301 uORF 3′ STOP: 355 leader length: 419 |
| 3262 | AGI number: AT5G03190.1 uORF ′5 STOP: 301 uORF 3′ STOP: 355 leader length: 419 |
| 3263 | AGI number: AT5G03240.3 uORF ′5 STOP: 178 uORF 3′ STOP: 274 leader length: 417 |
| 3264 | AGI number: AT5G03240.3 uORF ′5 STOP: 178 uORF 3′ STOP: 274 leader length: 417 |
| 3265 | AGI number: AT5G03240.3 uORF ′5 STOP: 93 uORF 3′ STOP: 258 leader length: 417 |
| 3266 | AGI number: AT5G03240.3 uORF ′5 STOP: 93 uORF 3′ STOP: 258 leader length: 417 |
| 3267 | AGI number: AT5G03280.1 uORF ′5 STOP: 98 uORF 3′ STOP: 284 leader length: 628 |
| 3268 | AGI number: AT5G03280.1 uORF ′5 STOP: 98 uORF 3′ STOP: 284 leader length: 628 |
| 3269 | AGI number: AT5G03330.1 uORF ′5 STOP: 157 uORF 3′ STOP: 211 leader length: 295 |
| 3270 | AGI number: AT5G03330.1 uORF ′5 STOP: 157 uORF 3′ STOP: 211 leader length: 295 |
| 3271 | AGI number: AT5G03500.3 uORF ′5 STOP: 49 uORF 3′ STOP: 106 leader length: 258 |
| 3272 | AGI number: AT5G03500.3 uORF ′5 STOP: 49 uORF 3′ STOP: 106 leader length: 258 |
| 3273 | AGI number: AT5G03540.2 uORF ′5 STOP: 259 uORF 3′ STOP: 322 leader length: 505 |
| 3274 | AGI number: AT5G03540.2 uORF ′5 STOP: 259 uORF 3′ STOP: 322 leader length: 505 |
| 3275 | AGI number: AT5G03795.1 uORF ′5 STOP: 58 uORF 3′ STOP: 112 leader length: 100 |
| 3276 | AGI number: AT5G03795.1 uORF ′5 STOP: 58 uORF 3′ STOP: 112 leader length: 100 |
| 3277 | AGI number: AT5G03990.1 uORF ′5 STOP: 119 uORF 3′ STOP: 176 leader length: 286 |
| 3278 | AGI number: AT5G03990.1 uORF ′5 STOP: 119 uORF 3′ STOP: 176 leader length: 286 |
| 3279 | AGI number: AT5G04220.1 uORF ′5 STOP: 388 uORF 3′ STOP: 448 leader length: 949 |
| 3280 | AGI number: AT5G04220.1 uORF ′5 STOP: 388 uORF 3′ STOP: 448 leader length: 949 |
| 3281 | AGI number: AT5G04320.1 uORF ′5 STOP: 72 uORF 3′ STOP: 138 leader length: 569 |
| 3282 | AGI number: AT5G04320.1 uORF ′5 STOP: 72 uORF 3′ STOP: 138 leader length: 569 |
| 3283 | AGI number: AT5G04320.1 uORF ′5 STOP: 94 uORF 3′ STOP: 163 leader length: 569 |
| 3284 | AGI number: AT5G04320.1 uORF ′5 STOP: 94 uORF 3′ STOP: 163 leader length: 569 |
| 3285 | AGI number: AT5G04320.2 uORF ′5 STOP: 73 uORF 3′ STOP: 139 leader length: 330 |
| 3286 | AGI number: AT5G04320.2 uORF ′5 STOP: 73 uORF 3′ STOP: 139 leader length: 330 |
| 3287 | AGI number: AT5G04320.2 uORF ′5 STOP: 95 uORF 3′ STOP: 164 leader length: 330 |
| 3288 | AGI number: AT5G04320.2 uORF ′5 STOP: 95 uORF 3′ STOP: 164 leader length: 330 |
| 3289 | AGI number: AT5G04480.2 uORF ′5 STOP: 32 uORF 3′ STOP: 128 leader length: 153 |
| 3290 | AGI number: AT5G04480.2 uORF ′5 STOP: 32 uORF 3′ STOP: 128 leader length: 153 |
| 3291 | AGI number: AT5G04480.2 uORF ′5 STOP: 58 uORF 3′ STOP: 115 leader length: 153 |
| 3292 | AGI number: AT5G04480.2 uORF ′5 STOP: 58 uORF 3′ STOP: 115 leader length: 153 |
| 3293 | AGI number: AT5G04920.1 uORF ′5 STOP: 159 uORF 3′ STOP: 240 leader length: 332 |
| 3294 | AGI number: AT5G04920.1 uORF ′5 STOP: 159 uORF 3′ STOP: 240 leader length: 332 |
| 3295 | AGI number: AT5G04940.1 uORF ′5 STOP: 158 uORF 3′ STOP: 236 leader length: 362 |
| 3296 | AGI number: AT5G04940.1 uORF ′5 STOP: 158 uORF 3′ STOP: 236 leader length: 362 |
| 3297 | AGI number: AT5G04940.1 uORF ′5 STOP: 187 uORF 3′ STOP: 253 leader length: 362 |
| 3298 | AGI number: AT5G04940.1 uORF ′5 STOP: 187 uORF 3′ STOP: 253 leader length: 362 |
| 3299 | AGI number: AT5G04940.2 uORF ′5 STOP: 158 uORF 3′ STOP: 236 leader length: 362 |
| 3300 | AGI number: AT5G04940.2 uORF ′5 STOP: 158 uORF 3′ STOP: 236 leader length: 362 |
| 3301 | AGI number: AT5G04940.2 uORF ′5 STOP: 187 uORF 3′ STOP: 253 leader length: 362 |
| 3302 | AGI number: AT5G04940.2 uORF ′5 STOP: 187 uORF 3′ STOP: 253 leader length: 362 |
| 3303 | AGI number: AT5G05210.2 uORF ′5 STOP: 120 uORF 3′ STOP: 189 leader length: 340 |
| 3304 | AGI number: AT5G05210.2 uORF ′5 STOP: 120 uORF 3′ STOP: 189 leader length: 340 |
| 3305 | AGI number: AT5G05210.2 uORF ′5 STOP: 70 uORF 3′ STOP: 145 leader length: 340 |
| 3306 | AGI number: AT5G05210.2 uORF ′5 STOP: 70 uORF 3′ STOP: 145 leader length: 340 |
| 3307 | AGI number: AT5G05350.1 uORF ′5 STOP: 162 uORF 3′ STOP: 306 leader length: 460 |
| 3308 | AGI number: AT5G05350.1 uORF ′5 STOP: 162 uORF 3′ STOP: 306 leader length: 460 |
| 3309 | AGI number: AT5G05350.1 uORF ′5 STOP: 459 uORF 3′ STOP: 615 leader length: 460 |
| 3310 | AGI number: AT5G05350.1 uORF ′5 STOP: 459 uORF 3′ STOP: 615 leader length: 460 |
| 3311 | AGI number: AT5G05930.2 uORF ′5 STOP: 422 uORF 3′ STOP: 482 leader length: 484 |
| 3312 | AGI number: AT5G05930.2 uORF ′5 STOP: 422 uORF 3′ STOP: 482 leader length: 484 |
| 3313 | AGI number: AT5G06100.3 uORF ′5 STOP: 29 uORF 3′ STOP: 101 leader length: 311 |
| 3314 | AGI number: AT5G06100.3 uORF ′5 STOP: 29 uORF 3′ STOP: 101 leader length: 311 |
| 3315 | AGI number: AT5G06130.1 uORF ′5 STOP: 214 uORF 3′ STOP: 301 leader length: 575 |
| 3316 | AGI number: AT5G06130.1 uORF ′5 STOP: 214 uORF 3′ STOP: 301 leader length: 575 |
| 3317 | AGI number: AT5G06130.1 uORF ′5 STOP: 207 uORF 3′ STOP: 327 leader length: 575 |
| 3318 | AGI number: AT5G06130.1 uORF ′5 STOP: 207 uORF 3′ STOP: 327 leader length: 575 |
| 3319 | AGI number: AT5G06265.3 uORF ′5 STOP: 26 uORF 3′ STOP: 197 leader length: 594 |
| 3320 | AGI number: AT5G06265.3 uORF ′5 STOP: 26 uORF 3′ STOP: 197 leader length: 594 |
| 3321 | AGI number: AT5G06265.3 uORF ′5 STOP: 96 uORF 3′ STOP: 192 leader length: 594 |
| 3322 | AGI number: AT5G06265.3 uORF ′5 STOP: 96 uORF 3′ STOP: 192 leader length: 594 |
| 3323 | AGI number: AT5G06265.3 uORF ′5 STOP: 103 uORF 3′ STOP: 166 leader length: 594 |
| 3324 | AGI number: AT5G06265.3 uORF ′5 STOP: 103 uORF 3′ STOP: 166 leader length: 594 |
| 3325 | AGI number: AT5G06420.2 uORF ′5 STOP: 539 uORF 3′ STOP: 602 leader length: 686 |
| 3326 | AGI number: AT5G06420.2 uORF ′5 STOP: 539 uORF 3′ STOP: 602 leader length: 686 |
| 3327 | AGI number: AT5G06750.1 uORF ′5 STOP: 244 uORF 3′ STOP: 361 leader length: 283 |
| 3328 | AGI number: AT5G06750.1 uORF ′5 STOP: 244 uORF 3′ STOP: 361 leader length: 283 |
| 3329 | AGI number: AT5G06839.2 uORF ′5 STOP: 161 uORF 3′ STOP: 461 leader length: 384 |
| 3330 | AGI number: AT5G06839.2 uORF ′5 STOP: 161 uORF 3′ STOP: 461 leader length: 384 |
| 3331 | AGI number: AT5G07370.4 uORF ′5 STOP: 26 uORF 3′ STOP: 209 leader length: 852 |
| 3332 | AGI number: AT5G07370.4 uORF ′5 STOP: 26 uORF 3′ STOP: 209 leader length: 852 |
| 3333 | AGI number: AT5G07370.4 uORF ′5 STOP: 115 uORF 3′ STOP: 190 leader length: 852 |
| 3334 | AGI number: AT5G07370.4 uORF ′5 STOP: 115 uORF 3′ STOP: 190 leader length: 852 |
| 3335 | AGI number: AT5G07370.4 uORF ′5 STOP: 348 uORF 3′ STOP: 417 leader length: 852 |
| 3336 | AGI number: AT5G07370.4 uORF ′5 STOP: 348 uORF 3′ STOP: 417 leader length: 852 |
| 3337 | AGI number: AT5G07790.1 uORF ′5 STOP: 37 uORF 3′ STOP: 130 leader length: 348 |
| 3338 | AGI number: AT5G07790.1 uORF ′5 STOP: 37 uORF 3′ STOP: 130 leader length: 348 |
| 3339 | AGI number: AT5G07840.1 uORF ′5 STOP: 256 uORF 3′ STOP: 394 leader length: 656 |
| 3340 | AGI number: AT5G07840.1 uORF ′5 STOP: 256 uORF 3′ STOP: 394 leader length: 656 |
| 3341 | AGI number: AT5G07840.1 uORF ′5 STOP: 284 uORF 3′ STOP: 407 leader length: 656 |
| 3342 | AGI number: AT5G07840.1 uORF ′5 STOP: 284 uORF 3′ STOP: 407 leader length: 656 |
| 3343 | AGI number: AT5G07842.1 uORF ′5 STOP: 318 uORF 3′ STOP: 456 leader length: 351 |
| 3344 | AGI number: AT5G07842.1 uORF ′5 STOP: 318 uORF 3′ STOP: 456 leader length: 351 |
| 3345 | AGI number: AT5G07890.2 uORF ′5 STOP: 229 uORF 3′ STOP: 310 leader length: 486 |
| 3346 | AGI number: AT5G07890.2 uORF ′5 STOP: 229 uORF 3′ STOP: 310 leader length: 486 |
| 3347 | AGI number: AT5G07920.1 uORF ′5 STOP: 282 uORF 3′ STOP: 354 leader length: 437 |
| 3348 | AGI number: AT5G07920.1 uORF ′5 STOP: 282 uORF 3′ STOP: 354 leader length: 437 |
| 3349 | AGI number: AT5G08100.2 uORF ′5 STOP: 122 uORF 3′ STOP: 293 leader length: 489 |
| 3350 | AGI number: AT5G08100.2 uORF ′5 STOP: 122 uORF 3′ STOP: 293 leader length: 489 |
| 3351 | AGI number: AT5G08130.4 uORF ′5 STOP: 44 uORF 3′ STOP: 206 leader length: 499 |
| 3352 | AGI number: AT5G08130.4 uORF ′5 STOP: 44 uORF 3′ STOP: 206 leader length: 499 |
| 3353 | AGI number: AT5G08130.5 uORF ′5 STOP: 44 uORF 3′ STOP: 206 leader length: 499 |
| 3354 | AGI number: AT5G08130.5 uORF ′5 STOP: 44 uORF 3′ STOP: 206 leader length: 499 |
| 3355 | AGI number: AT5G08130.5 uORF ′5 STOP: 270 uORF 3′ STOP: 381 leader length: 499 |
| 3356 | AGI number: AT5G08130.5 uORF ′5 STOP: 270 uORF 3′ STOP: 381 leader length: 499 |
| 3357 | AGI number: AT5G08185.3 uORF ′5 STOP: 31 uORF 3′ STOP: 121 leader length: 89 |
| 3358 | AGI number: AT5G08185.3 uORF ′5 STOP: 31 uORF 3′ STOP: 121 leader length: 89 |
| 3359 | AGI number: AT5G08560.1 uORF ′5 STOP: 164 uORF 3′ STOP: 293 leader length: 299 |
| 3360 | AGI number: AT5G08560.1 uORF ′5 STOP: 164 uORF 3′ STOP: 293 leader length: 299 |
| 3361 | AGI number: AT5G08560.1 uORF ′5 STOP: 204 uORF 3′ STOP: 288 leader length: 299 |
| 3362 | AGI number: AT5G08560.1 uORF ′5 STOP: 204 uORF 3′ STOP: 288 leader length: 299 |
| 3363 | AGI number: AT5G08560.1 uORF ′5 STOP: 268 uORF 3′ STOP: 334 leader length: 299 |
| 3364 | AGI number: AT5G08560.1 uORF ′5 STOP: 268 uORF 3′ STOP: 334 leader length: 299 |
| 3365 | AGI number: AT5G09350.1 uORF ′5 STOP: 120 uORF 3′ STOP: 189 leader length: 536 |
| 3366 | AGI number: AT5G09350.1 uORF ′5 STOP: 120 uORF 3′ STOP: 189 leader length: 536 |
| 3367 | AGI number: AT5G09460.1 uORF ′5 STOP: 152 uORF 3′ STOP: 284 leader length: 866 |
| 3368 | AGI number: AT5G09460.1 uORF ′5 STOP: 152 uORF 3′ STOP: 284 leader length: 866 |
| 3369 | AGI number: AT5G09460.1 uORF ′5 STOP: 241 uORF 3′ STOP: 412 leader length: 866 |
| 3370 | AGI number: AT5G09460.1 uORF ′5 STOP: 241 uORF 3′ STOP: 412 leader length: 866 |
| 3371 | AGI number: AT5G09460.1 uORF ′5 STOP: 607 uORF 3′ STOP: 682 leader length: 866 |
| 3372 | AGI number: AT5G09460.1 uORF ′5 STOP: 607 uORF 3′ STOP: 682 leader length: 866 |
| 3373 | AGI number: AT5G09461.1 uORF ′5 STOP: 241 uORF 3′ STOP: 412 leader length: 521 |
| 3374 | AGI number: AT5G09461.1 uORF ′5 STOP: 241 uORF 3′ STOP: 412 leader length: 521 |
| 3375 | AGI number: AT5G09462.1 uORF ′5 STOP: 152 uORF 3′ STOP: 284 leader length: 357 |
| 3376 | AGI number: AT5G09462.1 uORF ′5 STOP: 152 uORF 3′ STOP: 284 leader length: 357 |
| 3377 | AGI number: AT5G09600.2 uORF ′5 STOP: 66 uORF 3′ STOP: 159 leader length: 255 |
| 3378 | AGI number: AT5G09600.2 uORF ′5 STOP: 66 uORF 3′ STOP: 159 leader length: 255 |
| 3379 | AGI number: AT5G09630.1 uORF ′5 STOP: 25 uORF 3′ STOP: 106 leader length: 209 |
| 3380 | AGI number: AT5G09630.1 uORF ′5 STOP: 25 uORF 3′ STOP: 106 leader length: 209 |
| 3381 | AGI number: AT5G09670.1 uORF ′5 STOP: 219 uORF 3′ STOP: 279 leader length: 1167 |
| 3382 | AGI number: AT5G09670.1 uORF ′5 STOP: 219 uORF 3′ STOP: 279 leader length: 1167 |
| 3383 | AGI number: AT5G09670.1 uORF ′5 STOP: 226 uORF 3′ STOP: 289 leader length: 1167 |
| 3384 | AGI number: AT5G09670.1 uORF ′5 STOP: 226 uORF 3′ STOP: 289 leader length: 1167 |
| 3385 | AGI number: AT5G09680.2 uORF ′5 STOP: 33 uORF 3′ STOP: 93 leader length: 416 |
| 3386 | AGI number: AT5G09680.2 uORF ′5 STOP: 33 uORF 3′ STOP: 93 leader length: 416 |
| 3387 | AGI number: AT5G09850.1 uORF ′5 STOP: 264 uORF 3′ STOP: 372 leader length: 668 |
| 3388 | AGI number: AT5G09850.1 uORF ′5 STOP: 264 uORF 3′ STOP: 372 leader length: 668 |
| 3389 | AGI number: AT5G09850.1 uORF ′5 STOP: 527 uORF 3′ STOP: 584 leader length: 668 |
| 3390 | AGI number: AT5G09850.1 uORF ′5 STOP: 527 uORF 3′ STOP: 584 leader length: 668 |
| 3391 | AGI number: AT5G10030.1 uORF ′5 STOP: 26 uORF 3′ STOP: 146 leader length: 255 |
| 3392 | AGI number: AT5G10030.1 uORF ′5 STOP: 26 uORF 3′ STOP: 146 leader length: 255 |
| 3393 | AGI number: AT5G10030.1 uORF ′5 STOP: 51 uORF 3′ STOP: 165 leader length: 255 |
| 3394 | AGI number: AT5G10030.1 uORF ′5 STOP: 51 uORF 3′ STOP: 165 leader length: 255 |
| 3395 | AGI number: AT5G10510.2 uORF ′5 STOP: 438 uORF 3′ STOP: 531 leader length: 462 |
| 3396 | AGI number: AT5G10510.2 uORF ′5 STOP: 438 uORF 3′ STOP: 531 leader length: 462 |
| 3397 | AGI number: AT5G11150.1 uORF ′5 STOP: 64 uORF 3′ STOP: 154 leader length: 267 |
| 3398 | AGI number: AT5G11150.1 uORF ′5 STOP: 64 uORF 3′ STOP: 154 leader length: 267 |
| 3399 | AGI number: AT5G11730.1 uORF ′5 STOP: 348 uORF 3′ STOP: 432 leader length: 652 |
| 3400 | AGI number: AT5G11730.1 uORF ′5 STOP: 348 uORF 3′ STOP: 432 leader length: 652 |
| 3401 | AGI number: AT5G11860.3 uORF ′5 STOP: 75 uORF 3′ STOP: 132 leader length: 197 |
| 3402 | AGI number: AT5G11860.3 uORF ′5 STOP: 75 uORF 3′ STOP: 132 leader length: 197 |
| 3403 | AGI number: AT5G12440.3 uORF ′5 STOP: 13 uORF 3′ STOP: 64 leader length: 172 |
| 3404 | AGI number: AT5G12440.3 uORF ′5 STOP: 13 uORF 3′ STOP: 64 leader length: 172 |
| 3405 | AGI number: AT5G12480.1 uORF ′5 STOP: 12 uORF 3′ STOP: 102 leader length: 206 |
| 3406 | AGI number: AT5G12480.1 uORF ′5 STOP: 12 uORF 3′ STOP: 102 leader length: 206 |
| 3407 | AGI number: AT5G12840.2 uORF ′5 STOP: 176 uORF 3′ STOP: 230 leader length: 327 |
| 3408 | AGI number: AT5G12840.2 uORF ′5 STOP: 176 uORF 3′ STOP: 230 leader length: 327 |
| 3409 | AGI number: AT5G13290.1 uORF ′5 STOP: 262 uORF 3′ STOP: 430 leader length: 679 |
| 3410 | AGI number: AT5G13290.1 uORF ′5 STOP: 262 uORF 3′ STOP: 430 leader length: 679 |
| 3411 | AGI number: AT5G13660.2 uORF ′5 STOP: 183 uORF 3′ STOP: 282 leader length: 344 |
| 3412 | AGI number: AT5G13660.2 uORF ′5 STOP: 183 uORF 3′ STOP: 282 leader length: 344 |
| 3413 | AGI number: AT5G13660.2 uORF ′5 STOP: 203 uORF 3′ STOP: 287 leader length: 344 |
| 3414 | AGI number: AT5G13660.2 uORF ′5 STOP: 203 uORF 3′ STOP: 287 leader length: 344 |
| 3415 | AGI number: AT5G13840.1 uORF ′5 STOP: 137 uORF 3′ STOP: 320 leader length: 285 |
| 3416 | AGI number: AT5G13840.1 uORF ′5 STOP: 137 uORF 3′ STOP: 320 leader length: 285 |
| 3417 | AGI number: AT5G14310.1 uORF ′5 STOP: 56 uORF 3′ STOP: 188 leader length: 274 |
| 3418 | AGI number: AT5G14310.1 uORF ′5 STOP: 56 uORF 3′ STOP: 188 leader length: 274 |
| 3419 | AGI number: AT5G14310.1 uORF ′5 STOP: 120 uORF 3′ STOP: 183 leader length: 274 |
| 3420 | AGI number: AT5G14310.1 uORF ′5 STOP: 120 uORF 3′ STOP: 183 leader length: 274 |
| 3421 | AGI number: AT5G14720.1 uORF ′5 STOP: 83 uORF 3′ STOP: 137 leader length: 466 |
| 3422 | AGI number: AT5G14720.1 uORF ′5 STOP: 83 uORF 3′ STOP: 137 leader length: 466 |
| 3423 | AGI number: AT5G14740.1 uORF ′5 STOP: 98 uORF 3′ STOP: 284 leader length: 285 |
| 3424 | AGI number: AT5G14740.1 uORF ′5 STOP: 98 uORF 3′ STOP: 284 leader length: 285 |
| 3425 | AGI number: AT5G14740.3 uORF ′5 STOP: 4 uORF 3′ STOP: 94 leader length: 285 |
| 3426 | AGI number: AT5G14740.3 uORF ′5 STOP: 4 uORF 3′ STOP: 94 leader length: 285 |
| 3427 | AGI number: AT5G14740.5 uORF ′5 STOP: 4 uORF 3′ STOP: 94 leader length: 285 |
| 3428 | AGI number: AT5G14740.5 uORF ′5 STOP: 4 uORF 3′ STOP: 94 leader length: 285 |
| 3429 | AGI number: AT5G14740.5 uORF ′5 STOP: 38 uORF 3′ STOP: 101 leader length: 285 |
| 3430 | AGI number: AT5G14740.5 uORF ′5 STOP: 38 uORF 3′ STOP: 101 leader length: 285 |
| 3431 | AGI number: AT5G14740.5 uORF ′5 STOP: 98 uORF 3′ STOP: 284 leader length: 285 |
| 3432 | AGI number: AT5G14740.5 uORF ′5 STOP: 98 uORF 3′ STOP: 284 leader length: 285 |
| 3433 | AGI number: AT5G15020.2 uORF ′5 STOP: 141 uORF 3′ STOP: 222 leader length: 322 |
| 3434 | AGI number: AT5G15020.2 uORF ′5 STOP: 141 uORF 3′ STOP: 222 leader length: 322 |
| 3435 | AGI number: AT5G15260.1 uORF ′5 STOP: 118 uORF 3′ STOP: 235 leader length: 473 |
| 3436 | AGI number: AT5G15260.1 uORF ′5 STOP: 118 uORF 3′ STOP: 235 leader length: 473 |
| 3437 | AGI number: AT5G15260.1 uORF ′5 STOP: 288 uORF 3′ STOP: 348 leader length: 473 |
| 3438 | AGI number: AT5G15260.1 uORF ′5 STOP: 288 uORF 3′ STOP: 348 leader length: 473 |
| 3439 | AGI number: AT5G15260.1 uORF ′5 STOP: 332 uORF 3′ STOP: 386 leader length: 473 |
| 3440 | AGI number: AT5G15260.1 uORF ′5 STOP: 332 uORF 3′ STOP: 386 leader length: 473 |
| 3441 | AGI number: AT5G15320.2 uORF ′5 STOP: 103 uORF 3′ STOP: 247 leader length: 112 |
| 3442 | AGI number: AT5G15320.2 uORF ′5 STOP: 103 uORF 3′ STOP: 247 leader length: 112 |
| 3443 | AGI number: AT5G15400.1 uORF ′5 STOP: 16 uORF 3′ STOP: 124 leader length: 233 |
| 3444 | AGI number: AT5G15400.1 uORF ′5 STOP: 16 uORF 3′ STOP: 124 leader length: 233 |
| 3445 | AGI number: AT5G15580.1 uORF ′5 STOP: 66 uORF 3′ STOP: 126 leader length: 562 |
| 3446 | AGI number: AT5G15580.1 uORF ′5 STOP: 66 uORF 3′ STOP: 126 leader length: 562 |
| 3447 | AGI number: AT5G15790.1 uORF ′5 STOP: 149 uORF 3′ STOP: 236 leader length: 366 |
| 3448 | AGI number: AT5G15790.1 uORF ′5 STOP: 149 uORF 3′ STOP: 236 leader length: 366 |
| 3449 | AGI number: AT5G15948.1 uORF ′5 STOP: 196 uORF 3′ STOP: 361 leader length: 197 |
| 3450 | AGI number: AT5G15948.1 uORF ′5 STOP: 196 uORF 3′ STOP: 361 leader length: 197 |
| 3451 | AGI number: AT5G15950.1 uORF ′5 STOP: 153 uORF 3′ STOP: 351 leader length: 468 |
| 3452 | AGI number: AT5G15950.1 uORF ′5 STOP: 153 uORF 3′ STOP: 351 leader length: 468 |
| 3453 | AGI number: AT5G15950.1 uORF ′5 STOP: 193 uORF 3′ STOP: 358 leader length: 468 |
| 3454 | AGI number: AT5G15950.1 uORF ′5 STOP: 193 uORF 3′ STOP: 358 leader length: 468 |
| 3455 | AGI number: AT5G15950.1 uORF ′5 STOP: 272 uORF 3′ STOP: 338 leader length: 468 |
| 3456 | AGI number: AT5G15950.1 uORF ′5 STOP: 272 uORF 3′ STOP: 338 leader length: 468 |
| 3457 | AGI number: AT5G15950.2 uORF ′5 STOP: 247 uORF 3′ STOP: 433 leader length: 550 |
| 3458 | AGI number: AT5G15950.2 uORF ′5 STOP: 247 uORF 3′ STOP: 433 leader length: 550 |
| 3459 | AGI number: AT5G15950.2 uORF ′5 STOP: 275 uORF 3′ STOP: 440 leader length: 550 |
| 3460 | AGI number: AT5G15950.2 uORF ′5 STOP: 275 uORF 3′ STOP: 440 leader length: 550 |
| 3461 | AGI number: AT5G15950.2 uORF ′5 STOP: 354 uORF 3′ STOP: 420 leader length: 550 |
| 3462 | AGI number: AT5G15950.2 uORF ′5 STOP: 354 uORF 3′ STOP: 420 leader length: 550 |
| 3463 | AGI number: AT5G16140.2 uORF ′5 STOP: 40 uORF 3′ STOP: 118 leader length: 250 |
| 3464 | AGI number: AT5G16140.2 uORF ′5 STOP: 40 uORF 3′ STOP: 118 leader length: 250 |
| 3465 | AGI number: AT5G16140.2 uORF ′5 STOP: 108 uORF 3′ STOP: 159 leader length: 250 |
| 3466 | AGI number: AT5G16140.2 uORF ′5 STOP: 108 uORF 3′ STOP: 159 leader length: 250 |
| 3467 | AGI number: AT5G16280.2 uORF ′5 STOP: 107 uORF 3′ STOP: 245 leader length: 340 |
| 3468 | AGI number: AT5G16280.2 uORF ′5 STOP: 107 uORF 3′ STOP: 245 leader length: 340 |
| 3469 | AGI number: AT5G16510.2 uORF ′5 STOP: 70 uORF 3′ STOP: 124 leader length: 239 |
| 3470 | AGI number: AT5G16510.2 uORF ′5 STOP: 70 uORF 3′ STOP: 124 leader length: 239 |
| 3471 | AGI number: AT5G16610.2 uORF ′5 STOP: 69 uORF 3′ STOP: 135 leader length: 264 |
| 3472 | AGI number: AT5G16610.2 uORF ′5 STOP: 69 uORF 3′ STOP: 135 leader length: 264 |
| 3473 | AGI number: AT5G17460.1 uORF ′5 STOP: 334 uORF 3′ STOP: 442 leader length: 928 |
| 3474 | AGI number: AT5G17460.1 uORF ′5 STOP: 334 uORF 3′ STOP: 442 leader length: 928 |
| 3475 | AGI number: AT5G17460.1 uORF ′5 STOP: 138 uORF 3′ STOP: 393 leader length: 928 |
| 3476 | AGI number: AT5G17460.1 uORF ′5 STOP: 138 uORF 3′ STOP: 393 leader length: 928 |
| 3477 | AGI number: AT5G17460.1 uORF ′5 STOP: 311 uORF 3′ STOP: 458 leader length: 928 |
| 3478 | AGI number: AT5G17460.1 uORF ′5 STOP: 311 uORF 3′ STOP: 458 leader length: 928 |
| 3479 | AGI number: AT5G18590.2 uORF ′5 STOP: 160 uORF 3′ STOP: 283 leader length: 412 |
| 3480 | AGI number: AT5G18590.2 uORF ′5 STOP: 160 uORF 3′ STOP: 283 leader length: 412 |
| 3481 | AGI number: AT5G18690.1 uORF ′5 STOP: 52 uORF 3′ STOP: 424 leader length: 80 |
| 3482 | AGI number: AT5G18690.1 uORF ′5 STOP: 52 uORF 3′ STOP: 424 leader length: 80 |
| 3483 | AGI number: AT5G18860.1 uORF ′5 STOP: 131 uORF 3′ STOP: 245 leader length: 347 |
| 3484 | AGI number: AT5G18860.1 uORF ′5 STOP: 131 uORF 3′ STOP: 245 leader length: 347 |
| 3485 | AGI number: AT5G18860.1 uORF ′5 STOP: 154 uORF 3′ STOP: 250 leader length: 347 |
| 3486 | AGI number: AT5G18860.1 uORF ′5 STOP: 154 uORF 3′ STOP: 250 leader length: 347 |
| 3487 | AGI number: AT5G19420.2 uORF ′5 STOP: 70 uORF 3′ STOP: 154 leader length: 261 |
| 3488 | AGI number: AT5G19420.2 uORF ′5 STOP: 70 uORF 3′ STOP: 154 leader length: 261 |
| 3489 | AGI number: AT5G19430.2 uORF ′5 STOP: 122 uORF 3′ STOP: 413 leader length: 510 |
| 3490 | AGI number: AT5G19430.2 uORF ′5 STOP: 122 uORF 3′ STOP: 413 leader length: 510 |
| 3491 | AGI number: AT5G19670.1 uORF ′5 STOP: 127 uORF 3′ STOP: 217 leader length: 306 |
| 3492 | AGI number: AT5G19670.1 uORF ′5 STOP: 127 uORF 3′ STOP: 217 leader length: 306 |
| 3493 | AGI number: AT5G20250.1 uORF ′5 STOP: 20 uORF 3′ STOP: 305 leader length: 439 |
| 3494 | AGI number: AT5G20250.1 uORF ′5 STOP: 20 uORF 3′ STOP: 305 leader length: 439 |
| 3495 | AGI number: AT5G20450.3 uORF ′5 STOP: 135 uORF 3′ STOP: 249 leader length: 497 |
| 3496 | AGI number: AT5G20450.3 uORF ′5 STOP: 135 uORF 3′ STOP: 249 leader length: 497 |
| 3497 | AGI number: AT5G20680.1 uORF ′5 STOP: 144 uORF 3′ STOP: 207 leader length: 415 |
| 3498 | AGI number: AT5G20680.1 uORF ′5 STOP: 144 uORF 3′ STOP: 207 leader length: 415 |
| 3499 | AGI number: AT5G20680.1 uORF ′5 STOP: 163 uORF 3′ STOP: 229 leader length: 415 |
| 3500 | AGI number: AT5G20680.1 uORF ′5 STOP: 163 uORF 3′ STOP: 229 leader length: 415 |
| 3501 | AGI number: AT5G20930.1 uORF ′5 STOP: 156 uORF 3′ STOP: 207 leader length: 305 |
| 3502 | AGI number: AT5G20930.1 uORF ′5 STOP: 156 uORF 3′ STOP: 207 leader length: 305 |
| 3503 | AGI number: AT5G21910.1 uORF ′5 STOP: 61 uORF 3′ STOP: 352 leader length: 130 |
| 3504 | AGI number: AT5G21910.1 uORF ′5 STOP: 61 uORF 3′ STOP: 352 leader length: 130 |
| 3505 | AGI number: AT5G22130.2 uORF ′5 STOP: 106 uORF 3′ STOP: 175 leader length: 221 |
| 3506 | AGI number: AT5G22130.2 uORF ′5 STOP: 106 uORF 3′ STOP: 175 leader length: 221 |
| 3507 | AGI number: AT5G22940.1 uORF ′5 STOP: 37 uORF 3′ STOP: 187 leader length: 259 |
| 3508 | AGI number: AT5G22940.1 uORF ′5 STOP: 37 uORF 3′ STOP: 187 leader length: 259 |
| 3509 | AGI number: AT5G22940.1 uORF ′5 STOP: 120 uORF 3′ STOP: 171 leader length: 259 |
| 3510 | AGI number: AT5G22940.1 uORF ′5 STOP: 120 uORF 3′ STOP: 171 leader length: 259 |
| 3511 | AGI number: AT5G23450.2 uORF ′5 STOP: 186 uORF 3′ STOP: 306 leader length: 456 |
| 3512 | AGI number: AT5G23450.2 uORF ′5 STOP: 186 uORF 3′ STOP: 306 leader length: 456 |
| 3513 | AGI number: AT5G23450.3 uORF ′5 STOP: 186 uORF 3′ STOP: 306 leader length: 456 |
| 3514 | AGI number: AT5G23450.3 uORF ′5 STOP: 186 uORF 3′ STOP: 306 leader length: 456 |
| 3515 | AGI number: AT5G23610.2 uORF ′5 STOP: 179 uORF 3′ STOP: 248 leader length: 304 |
| 3516 | AGI number: AT5G23610.2 uORF ′5 STOP: 179 uORF 3′ STOP: 248 leader length: 304 |
| 3517 | AGI number: AT5G24105.1 uORF ′5 STOP: 149 uORF 3′ STOP: 383 leader length: 173 |
| 3518 | AGI number: AT5G24105.1 uORF ′5 STOP: 149 uORF 3′ STOP: 383 leader length: 173 |
| 3519 | AGI number: AT5G24360.3 uORF ′5 STOP: 205 uORF 3′ STOP: 262 leader length: 275 |
| 3520 | AGI number: AT5G24360.3 uORF ′5 STOP: 205 uORF 3′ STOP: 262 leader length: 275 |
| 3521 | AGI number: AT5G24760.2 uORF ′5 STOP: 36 uORF 3′ STOP: 99 leader length: 210 |
| 3522 | AGI number: AT5G24760.2 uORF ′5 STOP: 36 uORF 3′ STOP: 99 leader length: 210 |
| 3523 | AGI number: AT5G24970.2 uORF ′5 STOP: 111 uORF 3′ STOP: 177 leader length: 186 |
| 3524 | AGI number: AT5G24970.2 uORF ′5 STOP: 111 uORF 3′ STOP: 177 leader length: 186 |
| 3525 | AGI number: AT5G25110.1 uORF ′5 STOP: 83 uORF 3′ STOP: 134 leader length: 116 |
| 3526 | AGI number: AT5G25110.1 uORF ′5 STOP: 83 uORF 3′ STOP: 134 leader length: 116 |
| 3527 | AGI number: AT5G25560.3 uORF ′5 STOP: 371 uORF 3′ STOP: 422 leader length: 551 |
| 3528 | AGI number: AT5G25560.3 uORF ′5 STOP: 371 uORF 3′ STOP: 422 leader length: 551 |
| 3529 | AGI number: AT5G25560.4 uORF ′5 STOP: 476 uORF 3′ STOP: 545 leader length: 1505 |
| 3530 | AGI number: AT5G25560.4 uORF ′5 STOP: 476 uORF 3′ STOP: 545 leader length: 1505 |
| 3531 | AGI number: AT5G25620.1 uORF ′5 STOP: 200 uORF 3′ STOP: 350 leader length: 442 |
| 3532 | AGI number: AT5G25620.1 uORF ′5 STOP: 200 uORF 3′ STOP: 350 leader length: 442 |
| 3533 | AGI number: AT5G25620.2 uORF ′5 STOP: 292 uORF 3′ STOP: 442 leader length: 558 |
| 3534 | AGI number: AT5G25620.2 uORF ′5 STOP: 292 uORF 3′ STOP: 442 leader length: 558 |
| 3535 | AGI number: AT5G25760.2 uORF ′5 STOP: 56 uORF 3′ STOP: 164 leader length: 281 |
| 3536 | AGI number: AT5G25760.2 uORF ′5 STOP: 56 uORF 3′ STOP: 164 leader length: 281 |
| 3537 | AGI number: AT5G26622.1 uORF ′5 STOP: 305 uORF 3′ STOP: 368 leader length: 545 |
| 3538 | AGI number: AT5G26622.1 uORF ′5 STOP: 305 uORF 3′ STOP: 368 leader length: 545 |
| 3539 | AGI number: AT5G26740.3 uORF ′5 STOP: 132 uORF 3′ STOP: 201 leader length: 419 |
| 3540 | AGI number: AT5G26740.3 uORF ′5 STOP: 132 uORF 3′ STOP: 201 leader length: 419 |
| 3541 | AGI number: AT5G26910.1 uORF ′5 STOP: 239 uORF 3′ STOP: 335 leader length: 602 |
| 3542 | AGI number: AT5G26910.1 uORF ′5 STOP: 239 uORF 3′ STOP: 335 leader length: 602 |
| 3543 | AGI number: AT5G26910.3 uORF ′5 STOP: 173 uORF 3′ STOP: 269 leader length: 536 |
| 3544 | AGI number: AT5G26910.3 uORF ′5 STOP: 173 uORF 3′ STOP: 269 leader length: 536 |
| 3545 | AGI number: AT5G26940.4 uORF ′5 STOP: 50 uORF 3′ STOP: 158 leader length: 203 |
| 3546 | AGI number: AT5G26940.4 uORF ′5 STOP: 50 uORF 3′ STOP: 158 leader length: 203 |
| 3547 | AGI number: AT5G27950.1 uORF ′5 STOP: 40 uORF 3′ STOP: 184 leader length: 723 |
| 3548 | AGI number: AT5G27950.1 uORF ′5 STOP: 40 uORF 3′ STOP: 184 leader length: 723 |
| 3549 | AGI number: AT5G27950.1 uORF ′5 STOP: 181 uORF 3′ STOP: 238 leader length: 723 |
| 3550 | AGI number: AT5G27950.1 uORF ′5 STOP: 181 uORF 3′ STOP: 238 leader length: 723 |
| 3551 | AGI number: AT5G28850.1 uORF ′5 STOP: 766 uORF 3′ STOP: 859 leader length: 893 |
| 3552 | AGI number: AT5G28850.1 uORF ′5 STOP: 766 uORF 3′ STOP: 859 leader length: 893 |
| 3553 | AGI number: AT5G28850.1 uORF ′5 STOP: 158 uORF 3′ STOP: 875 leader length: 893 |
| 3554 | AGI number: AT5G28850.1 uORF ′5 STOP: 158 uORF 3′ STOP: 875 leader length: 893 |
| 3555 | AGI number: AT5G33210.2 uORF ′5 STOP: 8 uORF 3′ STOP: 104 leader length: 140 |
| 3556 | AGI number: AT5G33210.2 uORF ′5 STOP: 8 uORF 3′ STOP: 104 leader length: 140 |
| 3557 | AGI number: AT5G35980.1 uORF ′5 STOP: 368 uORF 3′ STOP: 425 leader length: 567 |
| 3558 | AGI number: AT5G35980.1 uORF ′5 STOP: 368 uORF 3′ STOP: 425 leader length: 567 |
| 3559 | AGI number: AT5G35980.1 uORF ′5 STOP: 382 uORF 3′ STOP: 442 leader length: 567 |
| 3560 | AGI number: AT5G35980.1 uORF ′5 STOP: 382 uORF 3′ STOP: 442 leader length: 567 |
| 3561 | AGI number: AT5G35980.2 uORF ′5 STOP: 368 uORF 3′ STOP: 425 leader length: 567 |
| 3562 | AGI number: AT5G35980.2 uORF ′5 STOP: 368 uORF 3′ STOP: 425 leader length: 567 |
| 3563 | AGI number: AT5G35980.2 uORF ′5 STOP: 382 uORF 3′ STOP: 442 leader length: 567 |
| 3564 | AGI number: AT5G35980.2 uORF ′5 STOP: 382 uORF 3′ STOP: 442 leader length: 567 |
| 3565 | AGI number: AT5G35995.2 uORF ′5 STOP: 81 uORF 3′ STOP: 198 leader length: 398 |
| 3566 | AGI number: AT5G35995.2 uORF ′5 STOP: 81 uORF 3′ STOP: 198 leader length: 398 |
| 3567 | AGI number: AT5G37370.4 uORF ′5 STOP: 85 uORF 3′ STOP: 163 leader length: 327 |
| 3568 | AGI number: AT5G37370.4 uORF ′5 STOP: 85 uORF 3′ STOP: 163 leader length: 327 |
| 3569 | AGI number: AT5G37380.2 uORF ′5 STOP: 26 uORF 3′ STOP: 104 leader length: 219 |
| 3570 | AGI number: AT5G37380.2 uORF ′5 STOP: 26 uORF 3′ STOP: 104 leader length: 219 |
| 3571 | AGI number: AT5G37380.3 uORF ′5 STOP: 20 uORF 3′ STOP: 122 leader length: 227 |
| 3572 | AGI number: AT5G37380.3 uORF ′5 STOP: 20 uORF 3′ STOP: 122 leader length: 227 |
| 3573 | AGI number: AT5G38590.2 uORF ′5 STOP: 93 uORF 3′ STOP: 198 leader length: 270 |
| 3574 | AGI number: AT5G38590.2 uORF ′5 STOP: 93 uORF 3′ STOP: 198 leader length: 270 |
| 3575 | AGI number: AT5G38970.2 uORF ′5 STOP: 137 uORF 3′ STOP: 278 leader length: 411 |
| 3576 | AGI number: AT5G38970.2 uORF ′5 STOP: 137 uORF 3′ STOP: 278 leader length: 411 |
| 3577 | AGI number: AT5G39360.1 uORF ′5 STOP: 92 uORF 3′ STOP: 173 leader length: 251 |
| 3578 | AGI number: AT5G39360.1 uORF ′5 STOP: 92 uORF 3′ STOP: 173 leader length: 251 |
| 3579 | AGI number: AT5G39450.1 uORF ′5 STOP: 117 uORF 3′ STOP: 222 leader length: 328 |
| 3580 | AGI number: AT5G39450.1 uORF ′5 STOP: 117 uORF 3′ STOP: 222 leader length: 328 |
| 3581 | AGI number: AT5G39450.1 uORF ′5 STOP: 163 uORF 3′ STOP: 229 leader length: 328 |
| 3582 | AGI number: AT5G39450.1 uORF ′5 STOP: 163 uORF 3′ STOP: 229 leader length: 328 |
| 3583 | AGI number: AT5G40370.2 uORF ′5 STOP: 38 uORF 3′ STOP: 167 leader length: 298 |
| 3584 | AGI number: AT5G40370.2 uORF ′5 STOP: 38 uORF 3′ STOP: 167 leader length: 298 |
| 3585 | AGI number: AT5G40760.1 uORF ′5 STOP: 47 uORF 3′ STOP: 104 leader length: 193 |
| 3586 | AGI number: AT5G40760.1 uORF ′5 STOP: 47 uORF 3′ STOP: 104 leader length: 193 |
| 3587 | AGI number: AT5G40810.2 uORF ′5 STOP: 111 uORF 3′ STOP: 204 leader length: 609 |
| 3588 | AGI number: AT5G40810.2 uORF ′5 STOP: 111 uORF 3′ STOP: 204 leader length: 609 |
| 3589 | AGI number: AT5G41110.1 uORF ′5 STOP: 362 uORF 3′ STOP: 434 leader length: 604 |
| 3590 | AGI number: AT5G41110.1 uORF ′5 STOP: 362 uORF 3′ STOP: 434 leader length: 604 |
| 3591 | AGI number: AT5G41410.1 uORF ′5 STOP: 164 uORF 3′ STOP: 305 leader length: 380 |
| 3592 | AGI number: AT5G41410.1 uORF ′5 STOP: 164 uORF 3′ STOP: 305 leader length: 380 |
| 3593 | AGI number: AT5G41610.1 uORF ′5 STOP: 123 uORF 3′ STOP: 261 leader length: 220 |
| 3594 | AGI number: AT5G41610.1 uORF ′5 STOP: 123 uORF 3′ STOP: 261 leader length: 220 |
| 3595 | AGI number: AT5G42560.3 uORF ′5 STOP: 192 uORF 3′ STOP: 255 leader length: 320 |
| 3596 | AGI number: AT5G42560.3 uORF ′5 STOP: 192 uORF 3′ STOP: 255 leader length: 320 |
| 3597 | AGI number: AT5G43320.1 uORF ′5 STOP: 197 uORF 3′ STOP: 365 leader length: 586 |
| 3598 | AGI number: AT5G43320.1 uORF ′5 STOP: 197 uORF 3′ STOP: 365 leader length: 586 |
| 3599 | AGI number: AT5G43320.1 uORF ′5 STOP: 285 uORF 3′ STOP: 348 leader length: 586 |
| 3600 | AGI number: AT5G43320.1 uORF ′5 STOP: 285 uORF 3′ STOP: 348 leader length: 586 |
| 3601 | AGI number: AT5G43810.2 uORF ′5 STOP: 151 uORF 3′ STOP: 259 leader length: 561 |
| 3602 | AGI number: AT5G43810.2 uORF ′5 STOP: 151 uORF 3′ STOP: 259 leader length: 561 |
| 3603 | AGI number: AT5G44040.1 uORF ′5 STOP: 83 uORF 3′ STOP: 155 leader length: 232 |
| 3604 | AGI number: AT5G44040.1 uORF ′5 STOP: 83 uORF 3′ STOP: 155 leader length: 232 |
| 3605 | AGI number: AT5G44568.1 uORF ′5 STOP: 23 uORF 3′ STOP: 269 leader length: 56 |
| 3606 | AGI number: AT5G44568.1 uORF ′5 STOP: 23 uORF 3′ STOP: 269 leader length: 56 |
| 3607 | AGI number: AT5G44800.1 uORF ′5 STOP: 45 uORF 3′ STOP: 141 leader length: 383 |
| 3608 | AGI number: AT5G44800.1 uORF ′5 STOP: 45 uORF 3′ STOP: 141 leader length: 383 |
| 3609 | AGI number: AT5G44980.1 uORF ′5 STOP: 18 uORF 3′ STOP: 162 leader length: 78 |
| 3610 | AGI number: AT5G44980.1 uORF ′5 STOP: 18 uORF 3′ STOP: 162 leader length: 78 |
| 3611 | AGI number: AT5G45100.2 uORF ′5 STOP: 103 uORF 3′ STOP: 244 leader length: 352 |
| 3612 | AGI number: AT5G45100.2 uORF ′5 STOP: 103 uORF 3′ STOP: 244 leader length: 352 |
| 3613 | AGI number: AT5G45100.2 uORF ′5 STOP: 86 uORF 3′ STOP: 209 leader length: 352 |
| 3614 | AGI number: AT5G45100.2 uORF ′5 STOP: 86 uORF 3′ STOP: 209 leader length: 352 |
| 3615 | AGI number: AT5G45430.1 uORF ′5 STOP: 196 uORF 3′ STOP: 328 leader length: 869 |
| 3616 | AGI number: AT5G45430.1 uORF ′5 STOP: 196 uORF 3′ STOP: 328 leader length: 869 |
| 3617 | AGI number: AT5G45430.2 uORF ′5 STOP: 15 uORF 3′ STOP: 369 leader length: 921 |
| 3618 | AGI number: AT5G45430.2 uORF ′5 STOP: 15 uORF 3′ STOP: 369 leader length: 921 |
| 3619 | AGI number: AT5G45430.2 uORF ′5 STOP: 248 uORF 3′ STOP: 380 leader length: 921 |
| 3620 | AGI number: AT5G45430.2 uORF ′5 STOP: 248 uORF 3′ STOP: 380 leader length: 921 |
| 3621 | AGI number: AT5G45430.2 uORF ′5 STOP: 649 uORF 3′ STOP: 784 leader length: 921 |
| 3622 | AGI number: AT5G45430.2 uORF ′5 STOP: 649 uORF 3′ STOP: 784 leader length: 921 |
| 3623 | AGI number: AT5G45470.1 uORF ′5 STOP: 41 uORF 3′ STOP: 134 leader length: 278 |
| 3624 | AGI number: AT5G45470.1 uORF ′5 STOP: 41 uORF 3′ STOP: 134 leader length: 278 |
| 3625 | AGI number: AT5G45470.1 uORF ′5 STOP: 54 uORF 3′ STOP: 162 leader length: 278 |
| 3626 | AGI number: AT5G45470.1 uORF ′5 STOP: 54 uORF 3′ STOP: 162 leader length: 278 |
| 3627 | AGI number: AT5G45710.2 uORF ′5 STOP: 240 uORF 3′ STOP: 522 leader length: 682 |
| 3628 | AGI number: AT5G45710.2 uORF ′5 STOP: 240 uORF 3′ STOP: 522 leader length: 682 |
| 3629 | AGI number: AT5G45710.2 uORF ′5 STOP: 382 uORF 3′ STOP: 481 leader length: 682 |
| 3630 | AGI number: AT5G45710.2 uORF ′5 STOP: 382 uORF 3′ STOP: 481 leader length: 682 |
| 3631 | AGI number: AT5G45820.1 uORF ′5 STOP: 33 uORF 3′ STOP: 84 leader length: 143 |
| 3632 | AGI number: AT5G45820.1 uORF ′5 STOP: 33 uORF 3′ STOP: 84 leader length: 143 |
| 3633 | AGI number: AT5G47050.1 uORF ′5 STOP: 104 uORF 3′ STOP: 212 leader length: 272 |
| 3634 | AGI number: AT5G47050.1 uORF ′5 STOP: 104 uORF 3′ STOP: 212 leader length: 272 |
| 3635 | AGI number: AT5G47070.1 uORF ′5 STOP: 95 uORF 3′ STOP: 149 leader length: 381 |
| 3636 | AGI number: AT5G47070.1 uORF ′5 STOP: 95 uORF 3′ STOP: 149 leader length: 381 |
| 3637 | AGI number: AT5G47100.1 uORF ′5 STOP: 199 uORF 3′ STOP: 256 leader length: 325 |
| 3638 | AGI number: AT5G47100.1 uORF ′5 STOP: 199 uORF 3′ STOP: 256 leader length: 325 |
| 3639 | AGI number: AT5G47220.1 uORF ′5 STOP: 21 uORF 3′ STOP: 171 leader length: 69 |
| 3640 | AGI number: AT5G47220.1 uORF ′5 STOP: 21 uORF 3′ STOP: 171 leader length: 69 |
| 3641 | AGI number: AT5G47250.1 uORF ′5 STOP: 43 uORF 3′ STOP: 196 leader length: 169 |
| 3642 | AGI number: AT5G47250.1 uORF ′5 STOP: 43 uORF 3′ STOP: 196 leader length: 169 |
| 3643 | AGI number: AT5G47430.1 uORF ′5 STOP: 77 uORF 3′ STOP: 203 leader length: 433 |
| 3644 | AGI number: AT5G47430.1 uORF ′5 STOP: 77 uORF 3′ STOP: 203 leader length: 433 |
| 3645 | AGI number: AT5G47430.2 uORF ′5 STOP: 45 uORF 3′ STOP: 120 leader length: 437 |
| 3646 | AGI number: AT5G47430.2 uORF ′5 STOP: 45 uORF 3′ STOP: 120 leader length: 437 |
| 3647 | AGI number: AT5G47430.3 uORF ′5 STOP: 80 uORF 3′ STOP: 155 leader length: 468 |
| 3648 | AGI number: AT5G47430.3 uORF ′5 STOP: 80 uORF 3′ STOP: 155 leader length: 468 |
| 3649 | AGI number: AT5G47430.3 uORF ′5 STOP: 112 uORF 3′ STOP: 238 leader length: 468 |
| 3650 | AGI number: AT5G47430.3 uORF ′5 STOP: 112 uORF 3′ STOP: 238 leader length: 468 |
| 3651 | AGI number: AT5G47520.1 uORF ′5 STOP: 145 uORF 3′ STOP: 199 leader length: 397 |
| 3652 | AGI number: AT5G47520.1 uORF ′5 STOP: 145 uORF 3′ STOP: 199 leader length: 397 |
| 3653 | AGI number: AT5G47710.1 uORF ′5 STOP: 4 uORF 3′ STOP: 61 leader length: 176 |
| 3654 | AGI number: AT5G47710.1 uORF ′5 STOP: 4 uORF 3′ STOP: 61 leader length: 176 |
| 3655 | AGI number: AT5G47720.5 uORF ′5 STOP: 45 uORF 3′ STOP: 126 leader length: 209 |
| 3656 | AGI number: AT5G47720.5 uORF ′5 STOP: 45 uORF 3′ STOP: 126 leader length: 209 |
| 3657 | AGI number: AT5G47730.2 uORF ′5 STOP: 425 uORF 3′ STOP: 530 leader length: 735 |
| 3658 | AGI number: AT5G47730.2 uORF ′5 STOP: 425 uORF 3′ STOP: 530 leader length: 735 |
| 3659 | AGI number: AT5G47730.2 uORF ′5 STOP: 152 uORF 3′ STOP: 281 leader length: 735 |
| 3660 | AGI number: AT5G47730.2 uORF ′5 STOP: 152 uORF 3′ STOP: 281 leader length: 735 |
| 3661 | AGI number: AT5G47730.2 uORF ′5 STOP: 393 uORF 3′ STOP: 513 leader length: 735 |
| 3662 | AGI number: AT5G47730.2 uORF ′5 STOP: 393 uORF 3′ STOP: 513 leader length: 735 |
| 3663 | AGI number: AT5G47970.1 uORF ′5 STOP: 164 uORF 3′ STOP: 269 leader length: 680 |
| 3664 | AGI number: AT5G47970.1 uORF ′5 STOP: 164 uORF 3′ STOP: 269 leader length: 680 |
| 3665 | AGI number: AT5G47970.1 uORF ′5 STOP: 247 uORF 3′ STOP: 307 leader length: 680 |
| 3666 | AGI number: AT5G47970.1 uORF ′5 STOP: 247 uORF 3′ STOP: 307 leader length: 680 |
| 3667 | AGI number: AT5G48220.3 uORF ′5 STOP: 132 uORF 3′ STOP: 387 leader length: 416 |
| 3668 | AGI number: AT5G48220.3 uORF ′5 STOP: 132 uORF 3′ STOP: 387 leader length: 416 |
| 3669 | AGI number: AT5G48220.3 uORF ′5 STOP: 323 uORF 3′ STOP: 395 leader length: 416 |
| 3670 | AGI number: AT5G48220.3 uORF ′5 STOP: 323 uORF 3′ STOP: 395 leader length: 416 |
| 3671 | AGI number: AT5G48230.1 uORF ′5 STOP: 227 uORF 3′ STOP: 344 leader length: 403 |
| 3672 | AGI number: AT5G48230.1 uORF ′5 STOP: 227 uORF 3′ STOP: 344 leader length: 403 |
| 3673 | AGI number: AT5G48580.1 uORF ′5 STOP: 61 uORF 3′ STOP: 241 leader length: 460 |
| 3674 | AGI number: AT5G48580.1 uORF ′5 STOP: 61 uORF 3′ STOP: 241 leader length: 460 |
| 3675 | AGI number: AT5G48580.1 uORF ′5 STOP: 174 uORF 3′ STOP: 267 leader length: 460 |
| 3676 | AGI number: AT5G48580.1 uORF ′5 STOP: 174 uORF 3′ STOP: 267 leader length: 460 |
| 3677 | AGI number: AT5G48610.1 uORF ′5 STOP: 233 uORF 3′ STOP: 311 leader length: 597 |
| 3678 | AGI number: AT5G48610.1 uORF ′5 STOP: 233 uORF 3′ STOP: 311 leader length: 597 |
| 3679 | AGI number: AT5G48610.1 uORF ′5 STOP: 240 uORF 3′ STOP: 303 leader length: 597 |
| 3680 | AGI number: AT5G48610.1 uORF ′5 STOP: 240 uORF 3′ STOP: 303 leader length: 597 |
| 3681 | AGI number: AT5G48610.1 uORF ′5 STOP: 244 uORF 3′ STOP: 298 leader length: 597 |
| 3682 | AGI number: AT5G48610.1 uORF ′5 STOP: 244 uORF 3′ STOP: 298 leader length: 597 |
| 3683 | AGI number: AT5G48655.3 uORF ′5 STOP: 40 uORF 3′ STOP: 115 leader length: 539 |
| 3684 | AGI number: AT5G48655.3 uORF ′5 STOP: 40 uORF 3′ STOP: 115 leader length: 539 |
| 3685 | AGI number: AT5G48660.1 uORF ′5 STOP: 108 uORF 3′ STOP: 174 leader length: 339 |
| 3686 | AGI number: AT5G48660.1 uORF ′5 STOP: 108 uORF 3′ STOP: 174 leader length: 339 |
| 3687 | AGI number: AT5G49470.1 uORF ′5 STOP: 945 uORF 3′ STOP: 1002 leader length: 1101 |
| 3688 | AGI number: AT5G49470.1 uORF ′5 STOP: 945 uORF 3′ STOP: 1002 leader length: 1101 |
| 3689 | AGI number: AT5G49610.1 uORF ′5 STOP: 28 uORF 3′ STOP: 121 leader length: 202 |
| 3690 | AGI number: AT5G49610.1 uORF ′5 STOP: 28 uORF 3′ STOP: 121 leader length: 202 |
| 3691 | AGI number: AT5G50010.1 uORF ′5 STOP: 12 uORF 3′ STOP: 507 leader length: 583 |
| 3692 | AGI number: AT5G50010.1 uORF ′5 STOP: 12 uORF 3′ STOP: 507 leader length: 583 |
| 3693 | AGI number: AT5G50010.1 uORF ′5 STOP: 226 uORF 3′ STOP: 295 leader length: 583 |
| 3694 | AGI number: AT5G50010.1 uORF ′5 STOP: 226 uORF 3′ STOP: 295 leader length: 583 |
| 3695 | AGI number: AT5G50010.1 uORF ′5 STOP: 559 uORF 3′ STOP: 688 leader length: 583 |
| 3696 | AGI number: AT5G50010.1 uORF ′5 STOP: 559 uORF 3′ STOP: 688 leader length: 583 |
| 3697 | AGI number: AT5G50012.1 uORF ′5 STOP: 12 uORF 3′ STOP: 507 leader length: 132 |
| 3698 | AGI number: AT5G50012.1 uORF ′5 STOP: 12 uORF 3′ STOP: 507 leader length: 132 |
| 3699 | AGI number: AT5G50180.1 uORF ′5 STOP: 158 uORF 3′ STOP: 365 leader length: 520 |
| 3700 | AGI number: AT5G50180.1 uORF ′5 STOP: 158 uORF 3′ STOP: 365 leader length: 520 |
| 3701 | AGI number: AT5G50400.1 uORF ′5 STOP: 18 uORF 3′ STOP: 141 leader length: 238 |
| 3702 | AGI number: AT5G50400.1 uORF ′5 STOP: 18 uORF 3′ STOP: 141 leader length: 238 |
| 3703 | AGI number: AT5G50400.1 uORF ′5 STOP: 82 uORF 3′ STOP: 148 leader length: 238 |
| 3704 | AGI number: AT5G50400.1 uORF ′5 STOP: 82 uORF 3′ STOP: 148 leader length: 238 |
| 3705 | AGI number: AT5G50430.1 uORF ′5 STOP: 4 uORF 3′ STOP: 79 leader length: 291 |
| 3706 | AGI number: AT5G50430.1 uORF ′5 STOP: 4 uORF 3′ STOP: 79 leader length: 291 |
| 3707 | AGI number: AT5G50430.3 uORF ′5 STOP: 4 uORF 3′ STOP: 79 leader length: 291 |
| 3708 | AGI number: AT5G50430.3 uORF ′5 STOP: 4 uORF 3′ STOP: 79 leader length: 291 |
| 3709 | AGI number: AT5G50565.1 uORF ′5 STOP: 55 uORF 3′ STOP: 265 leader length: 348 |
| 3710 | AGI number: AT5G50565.1 uORF ′5 STOP: 55 uORF 3′ STOP: 265 leader length: 348 |
| 3711 | AGI number: AT5G50565.1 uORF ′5 STOP: 165 uORF 3′ STOP: 243 leader length: 348 |
| 3712 | AGI number: AT5G50565.1 uORF ′5 STOP: 165 uORF 3′ STOP: 243 leader length: 348 |
| 3713 | AGI number: AT5G51060.1 uORF ′5 STOP: 44 uORF 3′ STOP: 134 leader length: 256 |
| 3714 | AGI number: AT5G51060.1 uORF ′5 STOP: 44 uORF 3′ STOP: 134 leader length: 256 |
| 3715 | AGI number: AT5G52030.1 uORF ′5 STOP: 429 uORF 3′ STOP: 495 leader length: 563 |
| 3716 | AGI number: AT5G52030.1 uORF ′5 STOP: 429 uORF 3′ STOP: 495 leader length: 563 |
| 3717 | AGI number: AT5G52040.4 uORF ′5 STOP: 121 uORF 3′ STOP: 220 leader length: 544 |
| 3718 | AGI number: AT5G52040.4 uORF ′5 STOP: 121 uORF 3′ STOP: 220 leader length: 544 |
| 3719 | AGI number: AT5G52530.1 uORF ′5 STOP: 25 uORF 3′ STOP: 106 leader length: 413 |
| 3720 | AGI number: AT5G52530.1 uORF ′5 STOP: 25 uORF 3′ STOP: 106 leader length: 413 |
| 3721 | AGI number: AT5G52530.1 uORF ′5 STOP: 245 uORF 3′ STOP: 299 leader length: 413 |
| 3722 | AGI number: AT5G52530.1 uORF ′5 STOP: 245 uORF 3′ STOP: 299 leader length: 413 |
| 3723 | AGI number: AT5G52530.2 uORF ′5 STOP: 3 uORF 3′ STOP: 84 leader length: 407 |
| 3724 | AGI number: AT5G52530.2 uORF ′5 STOP: 3 uORF 3′ STOP: 84 leader length: 407 |
| 3725 | AGI number: AT5G52550.1 uORF ′5 STOP: 117 uORF 3′ STOP: 333 leader length: 454 |
| 3726 | AGI number: AT5G52550.1 uORF ′5 STOP: 117 uORF 3′ STOP: 333 leader length: 454 |
| 3727 | AGI number: AT5G52860.1 uORF ′5 STOP: 9 uORF 3′ STOP: 72 leader length: 22 |
| 3728 | AGI number: AT5G52860.1 uORF ′5 STOP: 9 uORF 3′ STOP: 72 leader length: 22 |
| 3729 | AGI number: AT5G52890.2 uORF ′5 STOP: 69 uORF 3′ STOP: 174 leader length: 276 |
| 3730 | AGI number: AT5G52890.2 uORF ′5 STOP: 69 uORF 3′ STOP: 174 leader length: 276 |
| 3731 | AGI number: AT5G53180.2 uORF ′5 STOP: 395 uORF 3′ STOP: 500 leader length: 495 |
| 3732 | AGI number: AT5G53180.2 uORF ′5 STOP: 395 uORF 3′ STOP: 500 leader length: 495 |
| 3733 | AGI number: AT5G53250.1 uORF ′5 STOP: 89 uORF 3′ STOP: 293 leader length: 95 |
| 3734 | AGI number: AT5G53250.1 uORF ′5 STOP: 89 uORF 3′ STOP: 293 leader length: 95 |
| 3735 | AGI number: AT5G53550.1 uORF ′5 STOP: 110 uORF 3′ STOP: 224 leader length: 327 |
| 3736 | AGI number: AT5G53550.1 uORF ′5 STOP: 110 uORF 3′ STOP: 224 leader length: 327 |
| 3737 | AGI number: AT5G53550.1 uORF ′5 STOP: 136 uORF 3′ STOP: 235 leader length: 327 |
| 3738 | AGI number: AT5G53550.1 uORF ′5 STOP: 136 uORF 3′ STOP: 235 leader length: 327 |
| 3739 | AGI number: AT5G53550.2 uORF ′5 STOP: 136 uORF 3′ STOP: 319 leader length: 411 |
| 3740 | AGI number: AT5G53550.2 uORF ′5 STOP: 136 uORF 3′ STOP: 319 leader length: 411 |
| 3741 | AGI number: AT5G53590.1 uORF ′5 STOP: 49 uORF 3′ STOP: 163 leader length: 296 |
| 3742 | AGI number: AT5G53590.1 uORF ′5 STOP: 49 uORF 3′ STOP: 163 leader length: 296 |
| 3743 | AGI number: AT5G53620.2 uORF ′5 STOP: 17 uORF 3′ STOP: 77 leader length: 247 |
| 3744 | AGI number: AT5G53620.2 uORF ′5 STOP: 17 uORF 3′ STOP: 77 leader length: 247 |
| 3745 | AGI number: AT5G53760.1 uORF ′5 STOP: 77 uORF 3′ STOP: 206 leader length: 478 |
| 3746 | AGI number: AT5G53760.1 uORF ′5 STOP: 77 uORF 3′ STOP: 206 leader length: 478 |
| 3747 | AGI number: AT5G53760.1 uORF ′5 STOP: 114 uORF 3′ STOP: 210 leader length: 478 |
| 3748 | AGI number: AT5G53760.1 uORF ′5 STOP: 114 uORF 3′ STOP: 210 leader length: 478 |
| 3749 | AGI number: AT5G53880.1 uORF ′5 STOP: 78 uORF 3′ STOP: 306 leader length: 108 |
| 3750 | AGI number: AT5G53880.1 uORF ′5 STOP: 78 uORF 3′ STOP: 306 leader length: 108 |
| 3751 | AGI number: AT5G54130.3 uORF ′5 STOP: 159 uORF 3′ STOP: 219 leader length: 300 |
| 3752 | AGI number: AT5G54130.3 uORF ′5 STOP: 159 uORF 3′ STOP: 219 leader length: 300 |
| 3753 | AGI number: AT5G54650.2 uORF ′5 STOP: 205 uORF 3′ STOP: 268 leader length: 235 |
| 3754 | AGI number: AT5G54650.2 uORF ′5 STOP: 205 uORF 3′ STOP: 268 leader length: 235 |
| 3755 | AGI number: AT5G54670.1 uORF ′5 STOP: 90 uORF 3′ STOP: 165 leader length: 279 |
| 3756 | AGI number: AT5G54670.1 uORF ′5 STOP: 90 uORF 3′ STOP: 165 leader length: 279 |
| 3757 | AGI number: AT5G54730.1 uORF ′5 STOP: 10 uORF 3′ STOP: 154 leader length: 537 |
| 3758 | AGI number: AT5G54730.1 uORF ′5 STOP: 10 uORF 3′ STOP: 154 leader length: 537 |
| 3759 | AGI number: AT5G54760.3 uORF ′5 STOP: 64 uORF 3′ STOP: 148 leader length: 247 |
| 3760 | AGI number: AT5G54760.3 uORF ′5 STOP: 64 uORF 3′ STOP: 148 leader length: 247 |
| 3761 | AGI number: AT5G55120.1 uORF ′5 STOP: 152 uORF 3′ STOP: 338 leader length: 420 |
| 3762 | AGI number: AT5G55120.1 uORF ′5 STOP: 152 uORF 3′ STOP: 338 leader length: 420 |
| 3763 | AGI number: AT5G55120.1 uORF ′5 STOP: 274 uORF 3′ STOP: 331 leader length: 420 |
| 3764 | AGI number: AT5G55120.1 uORF ′5 STOP: 274 uORF 3′ STOP: 331 leader length: 420 |
| 3765 | AGI number: AT5G55600.1 uORF ′5 STOP: 136 uORF 3′ STOP: 220 leader length: 849 |
| 3766 | AGI number: AT5G55600.1 uORF ′5 STOP: 136 uORF 3′ STOP: 220 leader length: 849 |
| 3767 | AGI number: AT5G55600.1 uORF ′5 STOP: 738 uORF 3′ STOP: 795 leader length: 849 |
| 3768 | AGI number: AT5G55600.1 uORF ′5 STOP: 738 uORF 3′ STOP: 795 leader length: 849 |
| 3769 | AGI number: AT5G55850.2 uORF ′5 STOP: 68 uORF 3′ STOP: 218 leader length: 241 |
| 3770 | AGI number: AT5G55850.2 uORF ′5 STOP: 68 uORF 3′ STOP: 218 leader length: 241 |
| 3771 | AGI number: AT5G55850.3 uORF ′5 STOP: 72 uORF 3′ STOP: 222 leader length: 245 |
| 3772 | AGI number: AT5G55850.3 uORF ′5 STOP: 72 uORF 3′ STOP: 222 leader length: 245 |
| 3773 | AGI number: AT5G55910.1 uORF ′5 STOP: 5 uORF 3′ STOP: 140 leader length: 357 |
| 3774 | AGI number: AT5G55910.1 uORF ′5 STOP: 5 uORF 3′ STOP: 140 leader length: 357 |
| 3775 | AGI number: AT5G55910.1 uORF ′5 STOP: 28 uORF 3′ STOP: 133 leader length: 357 |
| 3776 | AGI number: AT5G55910.1 uORF ′5 STOP: 28 uORF 3′ STOP: 133 leader length: 357 |
| 3777 | AGI number: AT5G55970.2 uORF ′5 STOP: 129 uORF 3′ STOP: 279 leader length: 546 |
| 3778 | AGI number: AT5G55970.2 uORF ′5 STOP: 129 uORF 3′ STOP: 279 leader length: 546 |
| 3779 | AGI number: AT5G55970.2 uORF ′5 STOP: 199 uORF 3′ STOP: 274 leader length: 546 |
| 3780 | AGI number: AT5G55970.2 uORF ′5 STOP: 199 uORF 3′ STOP: 274 leader length: 546 |
| 3781 | AGI number: AT5G56250.2 uORF ′5 STOP: 120 uORF 3′ STOP: 285 leader length: 208 |
| 3782 | AGI number: AT5G56250.2 uORF ′5 STOP: 120 uORF 3′ STOP: 285 leader length: 208 |
| 3783 | AGI number: AT5G56790.1 uORF ′5 STOP: 117 uORF 3′ STOP: 240 leader length: 671 |
| 3784 | AGI number: AT5G56790.1 uORF ′5 STOP: 117 uORF 3′ STOP: 240 leader length: 671 |
| 3785 | AGI number: AT5G56900.1 uORF ′5 STOP: 317 uORF 3′ STOP: 473 leader length: 663 |
| 3786 | AGI number: AT5G56900.1 uORF ′5 STOP: 317 uORF 3′ STOP: 473 leader length: 663 |
| 3787 | AGI number: AT5G57015.1 uORF ′5 STOP: 33 uORF 3′ STOP: 201 leader length: 411 |
| 3788 | AGI number: AT5G57015.1 uORF ′5 STOP: 33 uORF 3′ STOP: 201 leader length: 411 |
| 3789 | AGI number: AT5G57050.1 uORF ′5 STOP: 229 uORF 3′ STOP: 358 leader length: 485 |
| 3790 | AGI number: AT5G57050.1 uORF ′5 STOP: 229 uORF 3′ STOP: 358 leader length: 485 |
| 3791 | AGI number: AT5G57050.1 uORF ′5 STOP: 236 uORF 3′ STOP: 314 leader length: 485 |
| 3792 | AGI number: AT5G57050.1 uORF ′5 STOP: 236 uORF 3′ STOP: 314 leader length: 485 |
| 3793 | AGI number: AT5G57180.1 uORF ′5 STOP: 33 uORF 3′ STOP: 99 leader length: 551 |
| 3794 | AGI number: AT5G57180.1 uORF ′5 STOP: 33 uORF 3′ STOP: 99 leader length: 551 |
| 3795 | AGI number: AT5G57180.1 uORF ′5 STOP: 202 uORF 3′ STOP: 307 leader length: 551 |
| 3796 | AGI number: AT5G57180.1 uORF ′5 STOP: 202 uORF 3′ STOP: 307 leader length: 551 |
| 3797 | AGI number: AT5G57180.2 uORF ′5 STOP: 26 uORF 3′ STOP: 131 leader length: 375 |
| 3798 | AGI number: AT5G57180.2 uORF ′5 STOP: 26 uORF 3′ STOP: 131 leader length: 375 |
| 3799 | AGI number: AT5G57180.3 uORF ′5 STOP: 33 uORF 3′ STOP: 99 leader length: 551 |
| 3800 | AGI number: AT5G57180.3 uORF ′5 STOP: 33 uORF 3′ STOP: 99 leader length: 551 |
| 3801 | AGI number: AT5G57180.3 uORF ′5 STOP: 202 uORF 3′ STOP: 307 leader length: 551 |
| 3802 | AGI number: AT5G57180.3 uORF ′5 STOP: 202 uORF 3′ STOP: 307 leader length: 551 |
| 3803 | AGI number: AT5G57290.1 uORF ′5 STOP: 75 uORF 3′ STOP: 486 leader length: 91 |
| 3804 | AGI number: AT5G57290.1 uORF ′5 STOP: 75 uORF 3′ STOP: 486 leader length: 91 |
| 3805 | AGI number: AT5G57290.2 uORF ′5 STOP: 75 uORF 3′ STOP: 402 leader length: 91 |
| 3806 | AGI number: AT5G57290.2 uORF ′5 STOP: 75 uORF 3′ STOP: 402 leader length: 91 |
| 3807 | AGI number: AT5G57290.3 uORF ′5 STOP: 75 uORF 3′ STOP: 483 leader length: 91 |
| 3808 | AGI number: AT5G57290.3 uORF ′5 STOP: 75 uORF 3′ STOP: 483 leader length: 91 |
| 3809 | AGI number: AT5G57340.1 uORF ′5 STOP: 328 uORF 3′ STOP: 403 leader length: 382 |
| 3810 | AGI number: AT5G57340.1 uORF ′5 STOP: 328 uORF 3′ STOP: 403 leader length: 382 |
| 3811 | AGI number: AT5G57550.1 uORF ′5 STOP: 4 uORF 3′ STOP: 157 leader length: 37 |
| 3812 | AGI number: AT5G57550.1 uORF ′5 STOP: 4 uORF 3′ STOP: 157 leader length: 37 |
| 3813 | AGI number: AT5G57565.1 uORF ′5 STOP: 100 uORF 3′ STOP: 199 leader length: 526 |
| 3814 | AGI number: AT5G57565.1 uORF ′5 STOP: 100 uORF 3′ STOP: 199 leader length: 526 |
| 3815 | AGI number: AT5G57610.1 uORF ′5 STOP: 248 uORF 3′ STOP: 380 leader length: 433 |
| 3816 | AGI number: AT5G57610.1 uORF ′5 STOP: 248 uORF 3′ STOP: 380 leader length: 433 |
| 3817 | AGI number: AT5G57790.1 uORF ′5 STOP: 48 uORF 3′ STOP: 153 leader length: 185 |
| 3818 | AGI number: AT5G57790.1 uORF ′5 STOP: 48 uORF 3′ STOP: 153 leader length: 185 |
| 3819 | AGI number: AT5G58200.1 uORF ′5 STOP: 18 uORF 3′ STOP: 225 leader length: 448 |
| 3820 | AGI number: AT5G58200.1 uORF ′5 STOP: 18 uORF 3′ STOP: 225 leader length: 448 |
| 3821 | AGI number: AT5G58200.1 uORF ′5 STOP: 121 uORF 3′ STOP: 208 leader length: 448 |
| 3822 | AGI number: AT5G58200.1 uORF ′5 STOP: 121 uORF 3′ STOP: 208 leader length: 448 |
| 3823 | AGI number: AT5G58200.1 uORF ′5 STOP: 125 uORF 3′ STOP: 197 leader length: 448 |
| 3824 | AGI number: AT5G58200.1 uORF ′5 STOP: 125 uORF 3′ STOP: 197 leader length: 448 |
| 3825 | AGI number: AT5G58370.1 uORF ′5 STOP: 45 uORF 3′ STOP: 186 leader length: 241 |
| 3826 | AGI number: AT5G58370.1 uORF ′5 STOP: 45 uORF 3′ STOP: 186 leader length: 241 |
| 3827 | AGI number: AT5G58540.3 uORF ′5 STOP: 100 uORF 3′ STOP: 364 leader length: 770 |
| 3828 | AGI number: AT5G58540.3 uORF ′5 STOP: 100 uORF 3′ STOP: 364 leader length: 770 |
| 3829 | AGI number: AT5G58930.1 uORF ′5 STOP: 231 uORF 3′ STOP: 300 leader length: 367 |
| 3830 | AGI number: AT5G58930.1 uORF ′5 STOP: 231 uORF 3′ STOP: 300 leader length: 367 |
| 3831 | AGI number: AT5G58960.3 uORF ′5 STOP: 40 uORF 3′ STOP: 100 leader length: 250 |
| 3832 | AGI number: AT5G58960.3 uORF ′5 STOP: 40 uORF 3′ STOP: 100 leader length: 250 |
| 3833 | AGI number: AT5G59010.2 uORF ′5 STOP: 184 uORF 3′ STOP: 244 leader length: 646 |
| 3834 | AGI number: AT5G59010.2 uORF ′5 STOP: 184 uORF 3′ STOP: 244 leader length: 646 |
| 3835 | AGI number: AT5G59370.2 uORF ′5 STOP: 360 uORF 3′ STOP: 483 leader length: 370 |
| 3836 | AGI number: AT5G59370.2 uORF ′5 STOP: 360 uORF 3′ STOP: 483 leader length: 370 |
| 3837 | AGI number: AT5G59430.2 uORF ′5 STOP: 51 uORF 3′ STOP: 117 leader length: 369 |
| 3838 | AGI number: AT5G59430.2 uORF ′5 STOP: 51 uORF 3′ STOP: 117 leader length: 369 |
| 3839 | AGI number: AT5G59780.1 uORF ′5 STOP: 16 uORF 3′ STOP: 229 leader length: 387 |
| 3840 | AGI number: AT5G59780.1 uORF ′5 STOP: 16 uORF 3′ STOP: 229 leader length: 387 |
| 3841 | AGI number: AT5G59780.2 uORF ′5 STOP: 2 uORF 3′ STOP: 215 leader length: 162 |
| 3842 | AGI number: AT5G59780.2 uORF ′5 STOP: 2 uORF 3′ STOP: 215 leader length: 162 |
| 3843 | AGI number: AT5G59950.5 uORF ′5 STOP: 47 uORF 3′ STOP: 125 leader length: 296 |
| 3844 | AGI number: AT5G59950.5 uORF ′5 STOP: 47 uORF 3′ STOP: 125 leader length: 296 |
| 3845 | AGI number: AT5G60050.1 uORF ′5 STOP: 104 uORF 3′ STOP: 218 leader length: 365 |
| 3846 | AGI number: AT5G60050.1 uORF ′5 STOP: 104 uORF 3′ STOP: 218 leader length: 365 |
| 3847 | AGI number: AT5G60100.3 uORF ′5 STOP: 269 uORF 3′ STOP: 401 leader length: 429 |
| 3848 | AGI number: AT5G60100.3 uORF ′5 STOP: 269 uORF 3′ STOP: 401 leader length: 429 |
| 3849 | AGI number: AT5G60100.3 uORF ′5 STOP: 346 uORF 3′ STOP: 412 leader length: 429 |
| 3850 | AGI number: AT5G60100.3 uORF ′5 STOP: 346 uORF 3′ STOP: 412 leader length: 429 |
| 3851 | AGI number: AT5G60100.3 uORF ′5 STOP: 357 uORF 3′ STOP: 420 leader length: 429 |
| 3852 | AGI number: AT5G60100.3 uORF ′5 STOP: 357 uORF 3′ STOP: 420 leader length: 429 |
| 3853 | AGI number: AT5G60450.1 uORF ′5 STOP: 98 uORF 3′ STOP: 323 leader length: 511 |
| 3854 | AGI number: AT5G60450.1 uORF ′5 STOP: 98 uORF 3′ STOP: 323 leader length: 511 |
| 3855 | AGI number: AT5G60548.1 uORF ′5 STOP: 278 uORF 3′ STOP: 380 leader length: 282 |
| 3856 | AGI number: AT5G60548.1 uORF ′5 STOP: 278 uORF 3′ STOP: 380 leader length: 282 |
| 3857 | AGI number: AT5G60550.1 uORF ′5 STOP: 253 uORF 3′ STOP: 361 leader length: 492 |
| 3858 | AGI number: AT5G60550.1 uORF ′5 STOP: 253 uORF 3′ STOP: 361 leader length: 492 |
| 3859 | AGI number: AT5G60550.1 uORF ′5 STOP: 278 uORF 3′ STOP: 380 leader length: 492 |
| 3860 | AGI number: AT5G60550.1 uORF ′5 STOP: 278 uORF 3′ STOP: 380 leader length: 492 |
| 3861 | AGI number: AT5G60570.1 uORF ′5 STOP: 222 uORF 3′ STOP: 345 leader length: 444 |
| 3862 | AGI number: AT5G60570.1 uORF ′5 STOP: 222 uORF 3′ STOP: 345 leader length: 444 |
| 3863 | AGI number: AT5G60570.1 uORF ′5 STOP: 253 uORF 3′ STOP: 328 leader length: 444 |
| 3864 | AGI number: AT5G60570.1 uORF ′5 STOP: 253 uORF 3′ STOP: 328 leader length: 444 |
| 3865 | AGI number: AT5G60580.3 uORF ′5 STOP: 66 uORF 3′ STOP: 309 leader length: 474 |
| 3866 | AGI number: AT5G60580.3 uORF ′5 STOP: 66 uORF 3′ STOP: 309 leader length: 474 |
| 3867 | AGI number: AT5G60690.1 uORF ′5 STOP: 182 uORF 3′ STOP: 311 leader length: 711 |
| 3868 | AGI number: AT5G60690.1 uORF ′5 STOP: 182 uORF 3′ STOP: 311 leader length: 711 |
| 3869 | AGI number: AT5G60750.1 uORF ′5 STOP: 35 uORF 3′ STOP: 86 leader length: 181 |
| 3870 | AGI number: AT5G60750.1 uORF ′5 STOP: 35 uORF 3′ STOP: 86 leader length: 181 |
| 3871 | AGI number: AT5G60890.1 uORF ′5 STOP: 37 uORF 3′ STOP: 154 leader length: 338 |
| 3872 | AGI number: AT5G60890.1 uORF ′5 STOP: 37 uORF 3′ STOP: 154 leader length: 338 |
| 3873 | AGI number: AT5G60940.2 uORF ′5 STOP: 48 uORF 3′ STOP: 375 leader length: 439 |
| 3874 | AGI number: AT5G60940.2 uORF ′5 STOP: 48 uORF 3′ STOP: 375 leader length: 439 |
| 3875 | AGI number: AT5G60940.2 uORF ′5 STOP: 221 uORF 3′ STOP: 371 leader length: 439 |
| 3876 | AGI number: AT5G60940.2 uORF ′5 STOP: 221 uORF 3′ STOP: 371 leader length: 439 |
| 3877 | AGI number: AT5G61010.1 uORF ′5 STOP: 419 uORF 3′ STOP: 533 leader length: 714 |
| 3878 | AGI number: AT5G61010.1 uORF ′5 STOP: 419 uORF 3′ STOP: 533 leader length: 714 |
| 3879 | AGI number: AT5G61010.1 uORF ′5 STOP: 189 uORF 3′ STOP: 342 leader length: 714 |
| 3880 | AGI number: AT5G61010.1 uORF ′5 STOP: 189 uORF 3′ STOP: 342 leader length: 714 |
| 3881 | AGI number: AT5G61010.1 uORF ′5 STOP: 456 uORF 3′ STOP: 528 leader length: 714 |
| 3882 | AGI number: AT5G61010.1 uORF ′5 STOP: 456 uORF 3′ STOP: 528 leader length: 714 |
| 3883 | AGI number: AT5G61010.1 uORF ′5 STOP: 463 uORF 3′ STOP: 574 leader length: 714 |
| 3884 | AGI number: AT5G61010.1 uORF ′5 STOP: 463 uORF 3′ STOP: 574 leader length: 714 |
| 3885 | AGI number: AT5G61010.2 uORF ′5 STOP: 372 uORF 3′ STOP: 486 leader length: 667 |
| 3886 | AGI number: AT5G61010.2 uORF ′5 STOP: 372 uORF 3′ STOP: 486 leader length: 667 |
| 3887 | AGI number: AT5G61010.2 uORF ′5 STOP: 409 uORF 3′ STOP: 481 leader length: 667 |
| 3888 | AGI number: AT5G61010.2 uORF ′5 STOP: 409 uORF 3′ STOP: 481 leader length: 667 |
| 3889 | AGI number: AT5G61010.2 uORF ′5 STOP: 416 uORF 3′ STOP: 527 leader length: 667 |
| 3890 | AGI number: AT5G61010.2 uORF ′5 STOP: 416 uORF 3′ STOP: 527 leader length: 667 |
| 3891 | AGI number: AT5G61230.1 uORF ′5 STOP: 235 uORF 3′ STOP: 385 leader length: 620 |
| 3892 | AGI number: AT5G61230.1 uORF ′5 STOP: 235 uORF 3′ STOP: 385 leader length: 620 |
| 3893 | AGI number: AT5G61230.1 uORF ′5 STOP: 332 uORF 3′ STOP: 389 leader length: 620 |
| 3894 | AGI number: AT5G61230.1 uORF ′5 STOP: 332 uORF 3′ STOP: 389 leader length: 620 |
| 3895 | AGI number: AT5G61380.1 uORF ′5 STOP: 298 uORF 3′ STOP: 370 leader length: 476 |
| 3896 | AGI number: AT5G61380.1 uORF ′5 STOP: 298 uORF 3′ STOP: 370 leader length: 476 |
| 3897 | AGI number: AT5G61420.1 uORF ′5 STOP: 34 uORF 3′ STOP: 175 leader length: 617 |
| 3898 | AGI number: AT5G61420.1 uORF ′5 STOP: 34 uORF 3′ STOP: 175 leader length: 617 |
| 3899 | AGI number: AT5G61540.2 uORF ′5 STOP: 299 uORF 3′ STOP: 353 leader length: 424 |
| 3900 | AGI number: AT5G61540.2 uORF ′5 STOP: 299 uORF 3′ STOP: 353 leader length: 424 |
| 3901 | AGI number: AT5G61540.3 uORF ′5 STOP: 288 uORF 3′ STOP: 342 leader length: 413 |
| 3902 | AGI number: AT5G61540.3 uORF ′5 STOP: 288 uORF 3′ STOP: 342 leader length: 413 |
| 3903 | AGI number: AT5G61570.1 uORF ′5 STOP: 12 uORF 3′ STOP: 69 leader length: 160 |
| 3904 | AGI number: AT5G61570.1 uORF ′5 STOP: 12 uORF 3′ STOP: 69 leader length: 160 |
| 3905 | AGI number: AT5G61960.1 uORF ′5 STOP: 136 uORF 3′ STOP: 436 leader length: 658 |
| 3906 | AGI number: AT5G61960.1 uORF ′5 STOP: 136 uORF 3′ STOP: 436 leader length: 658 |
| 3907 | AGI number: AT5G62090.1 uORF ′5 STOP: 30 uORF 3′ STOP: 117 leader length: 520 |
| 3908 | AGI number: AT5G62090.1 uORF ′5 STOP: 30 uORF 3′ STOP: 117 leader length: 520 |
| 3909 | AGI number: AT5G62130.2 uORF ′5 STOP: 204 uORF 3′ STOP: 417 leader length: 580 |
| 3910 | AGI number: AT5G62130.2 uORF ′5 STOP: 204 uORF 3′ STOP: 417 leader length: 580 |
| 3911 | AGI number: AT5G62130.2 uORF ′5 STOP: 308 uORF 3′ STOP: 380 leader length: 580 |
| 3912 | AGI number: AT5G62130.2 uORF ′5 STOP: 308 uORF 3′ STOP: 380 leader length: 580 |
| 3913 | AGI number: AT5G62620.1 uORF ′5 STOP: 46 uORF 3′ STOP: 97 leader length: 385 |
| 3914 | AGI number: AT5G62620.1 uORF ′5 STOP: 46 uORF 3′ STOP: 97 leader length: 385 |
| 3915 | AGI number: AT5G62640.3 uORF ′5 STOP: 169 uORF 3′ STOP: 232 leader length: 190 |
| 3916 | AGI number: AT5G62640.3 uORF ′5 STOP: 169 uORF 3′ STOP: 232 leader length: 190 |
| 3917 | AGI number: AT5G62760.1 uORF ′5 STOP: 63 uORF 3′ STOP: 546 leader length: 115 |
| 3918 | AGI number: AT5G62760.1 uORF ′5 STOP: 63 uORF 3′ STOP: 546 leader length: 115 |
| 3919 | AGI number: AT5G64130.2 uORF ′5 STOP: 114 uORF 3′ STOP: 219 leader length: 319 |
| 3920 | AGI number: AT5G64130.2 uORF ′5 STOP: 114 uORF 3′ STOP: 219 leader length: 319 |
| 3921 | AGI number: AT5G64130.2 uORF ′5 STOP: 295 uORF 3′ STOP: 565 leader length: 319 |
| 3922 | AGI number: AT5G64130.2 uORF ′5 STOP: 295 uORF 3′ STOP: 565 leader length: 319 |
| 3923 | AGI number: AT5G64150.1 uORF ′5 STOP: 116 uORF 3′ STOP: 326 leader length: 487 |
| 3924 | AGI number: AT5G64150.1 uORF ′5 STOP: 116 uORF 3′ STOP: 326 leader length: 487 |
| 3925 | AGI number: AT5G64250.1 uORF ′5 STOP: 47 uORF 3′ STOP: 119 leader length: 219 |
| 3926 | AGI number: AT5G64250.1 uORF ′5 STOP: 47 uORF 3′ STOP: 119 leader length: 219 |
| 3927 | AGI number: AT5G64250.1 uORF ′5 STOP: 103 uORF 3′ STOP: 160 leader length: 219 |
| 3928 | AGI number: AT5G64250.1 uORF ′5 STOP: 103 uORF 3′ STOP: 160 leader length: 219 |
| 3929 | AGI number: AT5G64340.1 uORF ′5 STOP: 450 uORF 3′ STOP: 702 leader length: 869 |
| 3930 | AGI number: AT5G64340.1 uORF ′5 STOP: 450 uORF 3′ STOP: 702 leader length: 869 |
| 3931 | AGI number: AT5G64340.1 uORF ′5 STOP: 186 uORF 3′ STOP: 300 leader length: 869 |
| 3932 | AGI number: AT5G64340.1 uORF ′5 STOP: 186 uORF 3′ STOP: 300 leader length: 869 |
| 3933 | AGI number: AT5G64340.1 uORF ′5 STOP: 382 uORF 3′ STOP: 448 leader length: 869 |
| 3934 | AGI number: AT5G64340.1 uORF ′5 STOP: 382 uORF 3′ STOP: 448 leader length: 869 |
| 3935 | AGI number: AT5G64341.1 uORF ′5 STOP: 186 uORF 3′ STOP: 300 leader length: 539 |
| 3936 | AGI number: AT5G64341.1 uORF ′5 STOP: 186 uORF 3′ STOP: 300 leader length: 539 |
| 3937 | AGI number: AT5G64341.1 uORF ′5 STOP: 382 uORF 3′ STOP: 448 leader length: 539 |
| 3938 | AGI number: AT5G64341.1 uORF ′5 STOP: 382 uORF 3′ STOP: 448 leader length: 539 |
| 3939 | AGI number: AT5G64342.1 uORF ′5 STOP: 186 uORF 3′ STOP: 300 leader length: 396 |
| 3940 | AGI number: AT5G64342.1 uORF ′5 STOP: 186 uORF 3′ STOP: 300 leader length: 396 |
| 3941 | AGI number: AT5G64460.5 uORF ′5 STOP: 159 uORF 3′ STOP: 246 leader length: 413 |
| 3942 | AGI number: AT5G64460.5 uORF ′5 STOP: 159 uORF 3′ STOP: 246 leader length: 413 |
| 3943 | AGI number: AT5G64550.1 uORF ′5 STOP: 42 uORF 3′ STOP: 282 leader length: 362 |
| 3944 | AGI number: AT5G64550.1 uORF ′5 STOP: 42 uORF 3′ STOP: 282 leader length: 362 |
| 3945 | AGI number: AT5G64960.2 uORF ′5 STOP: 108 uORF 3′ STOP: 324 leader length: 370 |
| 3946 | AGI number: AT5G64960.2 uORF ′5 STOP: 108 uORF 3′ STOP: 324 leader length: 370 |
| 3947 | AGI number: AT5G64960.2 uORF ′5 STOP: 145 uORF 3′ STOP: 277 leader length: 370 |
| 3948 | AGI number: AT5G64960.2 uORF ′5 STOP: 145 uORF 3′ STOP: 277 leader length: 370 |
| 3949 | AGI number: AT5G65180.1 uORF ′5 STOP: 77 uORF 3′ STOP: 179 leader length: 264 |
| 3950 | AGI number: AT5G65180.1 uORF ′5 STOP: 77 uORF 3′ STOP: 179 leader length: 264 |
| 3951 | AGI number: AT5G65310.1 uORF ′5 STOP: 150 uORF 3′ STOP: 303 leader length: 397 |
| 3952 | AGI number: AT5G65310.1 uORF ′5 STOP: 150 uORF 3′ STOP: 303 leader length: 397 |
| 3953 | AGI number: AT5G65310.1 uORF ′5 STOP: 217 uORF 3′ STOP: 310 leader length: 397 |
| 3954 | AGI number: AT5G65310.1 uORF ′5 STOP: 217 uORF 3′ STOP: 310 leader length: 397 |
| 3955 | AGI number: AT5G65740.1 uORF ′5 STOP: 82 uORF 3′ STOP: 199 leader length: 369 |
| 3956 | AGI number: AT5G65740.1 uORF ′5 STOP: 82 uORF 3′ STOP: 199 leader length: 369 |
| 3957 | AGI number: AT5G65740.1 uORF ′5 STOP: 330 uORF 3′ STOP: 483 leader length: 369 |
| 3958 | AGI number: AT5G65740.1 uORF ′5 STOP: 330 uORF 3′ STOP: 483 leader length: 369 |
| 3959 | AGI number: AT5G65740.3 uORF ′5 STOP: 82 uORF 3′ STOP: 175 leader length: 579 |
| 3960 | AGI number: AT5G65740.3 uORF ′5 STOP: 82 uORF 3′ STOP: 175 leader length: 579 |
| 3961 | AGI number: AT5G66005.1 uORF ′5 STOP: 42 uORF 3′ STOP: 219 leader length: 266 |
| 3962 | AGI number: AT5G66005.1 uORF ′5 STOP: 42 uORF 3′ STOP: 219 leader length: 266 |
| 3963 | AGI number: AT5G66005.1 uORF ′5 STOP: 76 uORF 3′ STOP: 271 leader length: 266 |
| 3964 | AGI number: AT5G66005.1 uORF ′5 STOP: 76 uORF 3′ STOP: 271 leader length: 266 |
| 3965 | AGI number: AT5G66040.2 uORF ′5 STOP: 107 uORF 3′ STOP: 176 leader length: 255 |
| 3966 | AGI number: AT5G66040.2 uORF ′5 STOP: 107 uORF 3′ STOP: 176 leader length: 255 |
| 3967 | AGI number: AT5G66040.2 uORF ′5 STOP: 215 uORF 3′ STOP: 512 leader length: 255 |
| 3968 | AGI number: AT5G66040.2 uORF ′5 STOP: 215 uORF 3′ STOP: 512 leader length: 255 |
| 3969 | AGI number: AT5G66050.2 uORF ′5 STOP: 2 uORF 3′ STOP: 401 leader length: 698 |
| 3970 | AGI number: AT5G66050.2 uORF ′5 STOP: 2 uORF 3′ STOP: 401 leader length: 698 |
| 3971 | AGI number: AT5G66110.1 uORF ′5 STOP: 42 uORF 3′ STOP: 114 leader length: 43 |
| 3972 | AGI number: AT5G66110.1 uORF ′5 STOP: 42 uORF 3′ STOP: 114 leader length: 43 |
| 3973 | AGI number: AT5G66250.2 uORF ′5 STOP: 190 uORF 3′ STOP: 256 leader length: 517 |
| 3974 | AGI number: AT5G66250.2 uORF ′5 STOP: 190 uORF 3′ STOP: 256 leader length: 517 |
| 3975 | AGI number: AT5G66310.1 uORF ′5 STOP: 2 uORF 3′ STOP: 185 leader length: 447 |
| 3976 | AGI number: AT5G66310.1 uORF ′5 STOP: 2 uORF 3′ STOP: 185 leader length: 447 |
| 3977 | AGI number: AT5G66420.1 uORF ′5 STOP: 45 uORF 3′ STOP: 174 leader length: 492 |
| 3978 | AGI number: AT5G66420.1 uORF ′5 STOP: 45 uORF 3′ STOP: 174 leader length: 492 |
| 3979 | AGI number: AT5G66600.1 uORF ′5 STOP: 287 uORF 3′ STOP: 398 leader length: 511 |
| 3980 | AGI number: AT5G66600.1 uORF ′5 STOP: 287 uORF 3′ STOP: 398 leader length: 511 |
| 3981 | AGI number: AT5G66600.1 uORF ′5 STOP: 337 uORF 3′ STOP: 427 leader length: 511 |
| 3982 | AGI number: AT5G66600.1 uORF ′5 STOP: 337 uORF 3′ STOP: 427 leader length: 511 |
| 3983 | AGI number: AT5G66730.1 uORF ′5 STOP: 100 uORF 3′ STOP: 232 leader length: 519 |
| 3984 | AGI number: AT5G66730.1 uORF ′5 STOP: 100 uORF 3′ STOP: 232 leader length: 519 |
| 3985 | AGI number: AT5G67440.2 uORF ′5 STOP: 599 uORF 3′ STOP: 761 leader length: 793 |
| 3986 | AGI number: AT5G67440.2 uORF ′5 STOP: 599 uORF 3′ STOP: 761 leader length: 793 |
| 3987 | AGI number: AT5G67600.1 uORF ′5 STOP: 45 uORF 3′ STOP: 408 leader length: 97 |
| 3988 | AGI number: AT5G67600.1 uORF ′5 STOP: 45 uORF 3′ STOP: 408 leader length: 97 |
| 3989 | AGI number: AT5G67620.1 uORF ′5 STOP: 69 uORF 3′ STOP: 159 leader length: 276 |
| 3990 | AGI number: AT5G67620.1 uORF ′5 STOP: 69 uORF 3′ STOP: 159 leader length: 276 |
| 3991 | AGI number: AT5G67620.1 uORF ′5 STOP: 89 uORF 3′ STOP: 146 leader length: 276 |
| 3992 | AGI number: AT5G67620.1 uORF ′5 STOP: 89 uORF 3′ STOP: 146 leader length: 276 |
| 3993 | AGI number: ATMG00410.1 uORF ′5 STOP: 3 uORF 3′ STOP: 87 leader length: 163 |
| 3994 | AGI number: ATMG00410.1 uORF ′5 STOP: 3 uORF 3′ STOP: 87 leader length: 163 |
| 3995 | AGI number: ATMG00730.1 uORF ′5 STOP: 269 uORF 3′ STOP: 323 leader length: 649 |
| 3996 | AGI number: ATMG00730.1 uORF ′5 STOP: 269 uORF 3′ STOP: 323 leader length: 649 |
| 3997 | AGI number: ATMG00730.1 uORF ′5 STOP: 504 uORF 3′ STOP: 570 leader length: 649 |
| 3998 | AGI number: ATMG00730.1 uORF ′5 STOP: 504 uORF 3′ STOP: 570 leader length: 649 |
| 3999 | AT1G01030.1 Symbols: NGA3: NGATHA3. Chr1: 11864-12940 REVERSE LENGTH = 358 |
| 4000 | AT1G01060.1 Symbols: LHY1, LHY: LATE ELONGATED HYPOCOTYL, LATE ELONGATED |
| | HYPOCOTYL 1. Chr1: 33992-37061 REVERSE LENGTH = 645 |
| 4001 | AT1G01160.2 Symbols: GIF2: GRF1-interacting factor 2. Chr1: 72583-73883 FORWARD |
| | LENGTH = 229 |
| 4002 | AT1G01340.2 Symbols: ACBK1, CNGC10, ATCNGC10: cyclic nucleotide gated channel 10. |
| | Chr1: 132414-135216 REVERSE LENGTH = 711 |
| 4003 | AT1G01650.1 Symbols: ATSPPL4, SPPL4: SIGNAL PEPTIDE PEPTIDASE-LIKE 4, ARABIDOPSIS |
| | THALIANA SIGNAL PEPTIDE PEPTIDASE-LIKE 4. Chr1: 233188-237647 REVERSE LENGTH = 540 |
| 4004 | AT1G01780.1 Symbols: PLIM2b: PLIM2b. Chr1: 282919-284082 FORWARD LENGTH = 205 |
| 4005 | AT1G01880.1 Symbols: AtGEN1, GEN1: ortholog of HsGEN1. Chr1: 306558-308991 REVERSE |
| | LENGTH = 599 |
| 4006 | AT1G02010.1 Symbols: SEC1A: secretory 1A. Chr1: 348046-352197 FORWARD LENGTH = 673 |
| 4007 | AT1G02145.3 Symbols: ALG12, EBS4: EMS-MUTAGENIZED BRI1(BRASSINOSTEROID |
| | INSENSITIVE 1) SUPPRESSOR 4, homolog of asparagine-linked glycosylation 12. Chr1: 404627- |
| | 408485 FORWARD LENGTH = 497 |
| 4008 | AT1G02205.3 Symbols: CER22, CER1: ECERIFERUM 22, ECERIFERUM 1. Chr1: 418818-422154 |
| | FORWARD LENGTH = 630 |
| 4009 | AT1G02260.1 Symbols: no symbol available: no full name available. Chr1: 440757-442459 REVERSE |
| | LENGTH = 502 |
| 4010 | AT1G02290.1 Symbols: no symbol available: no full name available. Chr1: 450646-451977 REVERSE |
| | LENGTH = 443 |
| 4011 | AT1G02380.1 Symbols: no symbol available: no full name available. Chr1: 477814-478850 FORWARD |
| | LENGTH = 231 |
| 4012 | AT1G02720.1 Symbols: GATL5, AtGATL5: galacturonosyltransferase-like 5. Chr1: 592115-593200 |
| | FORWARD LENGTH = 361 |
| 4013 | AT1G02880.3 Symbols: TPK1: thiamin pyrophosphokinase1. Chr1: 643063-644485 REVERSE |
| | LENGTH = 267 |
| 4014 | AT1G02930.1 Symbols: ATGSTF3, ATGSTF6, GST1, ERD11, GSTF6, ATGSTI: EARLY RESPONSIVE |
| | TO DEHYDRATION 11, ARABIDOPSIS GLUTATHIONE S-TRANSFERASE 1, ARABIDOPSIS |
| | THALIANA GLUATIONE S-TRANSFERASE F3, glutathione S-transferase 6, GLUTATHIONE S- |
| | TRANSFERASE 1, GLUTATHIONE S-TRANSFERASE. Chr1: 661363-662191 REVERSE |
| | LENGTH = 208 |
| 4015 | AT1G03080.1 Symbols: NET1D: Networked 1D. Chr1: 731794-737332 REVERSE LENGTH = 1733 |
| 4016 | AT1G03310.2 Symbols: ATISA2, BE2, DBE1, ISA2, AtBE2: debranching enzyme 1, BRANCHING |
| | ENZYME 2, ARABIDOPSIS THALIANA ISOAMYLASE 2. Chr1: 813975-816623 FORWARD |
| | LENGTH = 882 |
| 4017 | AT1G03860.1 Symbols: PHB2, ATPHB2: prohibitin 2. Chr1: 979611-981157 REVERSE LENGTH = 286 |
| 4018 | AT1G03930.1 Symbols: CKL9ALPHA, CKL9BETA, ADK1: dual specificity kinase 1, CASEIN KINASE |
| | I-LIKE 9 ALPHA, CASEIN KINASE I-LIKE 9 BETA. Chr1: 1005439-1008118 FORWARD |
| | LENGTH = 471 |
| 4019 | AT1G03960.1 Symbols: no symbol available: no full name available. Chr1: 1014085-1017472 |
| | FORWARD LENGTH = 529 |
| 4020 | AT1G03980.1 Symbols: PCS2, ATPCS2: phytochelatin synthase 2. Chr1: 1019311-1021194 REVERSE |
| | LENGTH = 367 |
| 4021 | AT1G04120.1 Symbols: ABCC5, ATABCC5, MRP5, ATMRP5: Arabidopsis thaliana ATP-binding |
| | cassette C5, multidrug resistance-associated protein 5, MULTIDRUG RESISTANCE PROTEIN 5, ATP- |
| | binding cassette C5. Chr1: 1064848-1070396 REVERSE LENGTH = 1514 |
| 4022 | AT1G04140.2 Symbols: no symbol available: no full name available. Chr1: 1075992-1080321 |
| | REVERSE LENGTH = 793 |
| 4023 | AT1G04300.3 Symbols: no symbol available: no full name available. Chr1: 1148818-1153895 |
| | REVERSE LENGTH = 1082 |
| 4024 | AT1G04440.1 Symbols: CKL13: casein kinase like 13. Chr1: 1202815-1205664 FORWARD |
| | LENGTH = 468 |
| 4025 | AT1G04778.1 Symbols: no symbol available: no full name available. Chr1: 1339947-1340117 |
| | FORWARD LENGTH = 56 |
| 4026 | AT1G04960.2 Symbols: no symbol available: no full name available. Chr1: 1408021-1410424 |
| | REVERSE LENGTH = 334 |
| 4027 | AT1G05160.1 Symbols: KAO1, ATKAO1, CYP88A3: ENT-KAURENOIC ACID OXYDASE |
| | 1, “cytochrome P450, family 88, subfamily A, polypeptide 3”. Chr1: 1487640-1489828 REVERSE |
| | LENGTH = 490 |
| 4028 | AT1G05200.1 Symbols: GLR3.4, ATGLR3.4, GLUR3: glutamate receptor 3.4. Chr1: 1505642-1509002 |
| | FORWARD LENGTH = 959 |
| 4029 | AT1G05230.1 Symbols: HDG2: homeodomain GLABROUS 2. Chr1: 1513388-1517024 REVERSE |
| | LENGTH = 721 |
| 4030 | AT1G05690.1 Symbols: BT3: BTB and TAZ domain protein 3. Chr1: 1707809-1709132 FORWARD |
| | LENGTH = 364 |
| 4031 | AT1G05710.2 Symbols: no symbol available: no full name available. Chr1: 1716198-1717023 |
| | FORWARD LENGTH = 171 |
| 4032 | AT1G05730.1 Symbols: no symbol available: no full name available. Chr1: 1719107-1720159 |
| | FORWARD LENGTH = 149 |
| 4033 | AT1G06150.1 Symbols: EMB1444, LHL1: LONESOME HIGHWAY LIKE 1, EMBRYO DEFECTIVE |
| | 1444. Chr1: 1869228-1873194 REVERSE LENGTH = 734 |
| 4034 | AT1G06270.1 Symbols: no symbol available: no full name available. Chr1: 1918242-1919273 |
| | REVERSE LENGTH = 343 |
| 4035 | AT1G06780.2 Symbols: GAUT6: galacturonosyltransferase 6. Chr1: 2083689-2086853 FORWARD |
| | LENGTH = 602 |
| 4036 | AT1G07030.1 Symbols: no symbol available: no full name available. Chr1: 2158631-2160524 |
| | REVERSE LENGTH = 326 |
| 4037 | AT1G07590.1 Symbols: no symbol available: no full name available. Chr1: 2336649-2338481 |
| | REVERSE LENGTH = 534 |
| 4038 | AT1G07700.3 Symbols: no symbol available: no full name available. Chr1: 2380297-2381127 |
| | FORWARD LENGTH = 217 |
| 4039 | AT1G07780.3 Symbols: PAI1, TRP6: TRANSIENT RECEPTOR POTENTIAL |
| | 6, phosphoribosy lanthranilate isomerase 1. Chr1: 2410352-2411833 REVERSE LENGTH = 275 |
| 4040 | AT1G07960.2 Symbols: ATPDIL5-1, PDIL5-1: PDI-like 5-1. Chrl: 2467681-2468831 FORWARD |
| | LENGTH = 146 |
| 4041 | AT1G08030.1 Symbols: AQC1, HPS7, TPST: hypersensitive to Pi starvation 7, active quiescent |
| | center1, tyrosylprotein sulfotransferase. Chr1: 2489575-2492741 REVERSE LENGTH = 500 |
| 4042 | AT1G08600.3 Symbols: ATRX, CHR20.: Chr1: 2724562-2733431 FORWARD LENGTH = 1479 |
| 4043 | AT1G08620.1 Symbols: PKDM7D.: Chr1: 2737554-2743370 FORWARD LENGTH = 1209 |
| 4044 | AT1G08820.1 Symbols: VAP27-2: vamp/synaptobrevin-associated protein 27-2. Chr1: 2821810-2824412 |
| | REVERSE LENGTH = 386 |
| 4045 | AT1G09180.1 Symbols: ATSARA1A, SARA1A, ATSAR1: SECRETION-ASSOCIATED RAS |
| | 1, secretion-associated RAS super family 1. Chr1: 2965147-2965941 FORWARD LENGTH = 193 |
| 4046 | AT1G09300.1 Symbols: AtICP55.: Chr1: 3003981-3008131 FORWARD LENGTH = 493 |
| 4047 | AT1G09840.1 Symbols: ATSK41, SK41, HIR1, AtHIR1: hypersensitive induced reaction 1, shaggy-like |
| | protein kinase 41. Chr1: 3196114-3199524 REVERSE LENGTH = 421 |
| 4048 | AT1G10120.1 Symbols: CIB4: CRY2-interacting bHLH 4. Chr1: 3304228-3305984 REVERSE |
| | LENGTH = 366 |
| 4049 | AT1G10522.1 Symbols: PRIN2: PLASTID REDOX INSENSITIVE 2. Chr1: 3470009-3471291 |
| | FORWARD LENGTH = 179 |
| 4050 | AT1G10650.1 Symbols: no symbol available: no full name available. Chr1: 3524122-3525318 |
| | REVERSE LENGTH = 339 |
| 4051 | AT1G10740.1 Symbols: no symbol available: no full name available. Chr1: 3568343-3570466 |
| | REVERSE LENGTH = 473 |
| 4052 | AT1G11170.1 Symbols: no symbol available: no full name available. Chr1: 3741724-3744459 |
| | FORWARD LENGTH = 438 |
| 4053 | AT1G11270.1 Symbols: no symbol available: no full name available. Chr1: 3785715-3786653 |
| | REVERSE LENGTH = 312 |
| 4054 | AT1G11450.2 Symbols: UMAMIT27: Usually multiple acids move in and out Transporters 27. |
| | Chr1: 3853470-3855259 FORWARD LENGTH = 301 |
| 4055 | AT1G11755.1 Symbols: LEW1: LEAF WILTING 1. Chr1: 3969987-3971488 REVERSE LENGTH = 254 |
| 4056 | AT1G11790.1 Symbols: AtADTI, ADT1: arogenate dehydratase 1, Arabidopsis thaliana arogenate |
| | dehydratase 1. Chr1: 3981476-3984962 FORWARD LENGTH = 392 |
| 4057 | AT1G12260.1 Symbols: NAC007, VND4, ANAC007, EMB2749: NAC 007, VASCULAR RELATED |
| | NAC-DOMAIN PROTEIN 4, EMBRYO DEFECTIVE 2749. Chr1: 4163058-4164486 REVERSE |
| | LENGTH = 395 |
| 4058 | AT1G12380.1 Symbols: no symbol available: no full name available. Chr1: 4214499-4216880 |
| | REVERSE LENGTH = 793 |
| 4059 | AT1G12440.1 Symbols: no symbol available: no full name available. Chr1: 4241816-4242322 |
| | REVERSE LENGTH = 168 |
| 4060 | AT1G12520.1 Symbols: ATCCS, CCS: copper chaperone for SOD1. Chr1: 4267277-4268900 REVERSE |
| | LENGTH = 320 |
| 4061 | AT1G12800.1 Symbols: SDP: S1 domain-containing RBP. Chr1: 4361778-4365189 REVERSE |
| | LENGTH = 767 |
| 4062 | AT1G12900.1 Symbols: GAPA-2: glyceraldehyde 3-phosphate dehydrogenase A subunit 2. |
| | Chr1: 4392634-4394283 REVERSE LENGTH = 399 |
| 4063 | AT1G12990.1 Symbols: no symbol available: no full name available. Chr1: 4433973-4435552 |
| | FORWARD LENGTH = 392 |
| 4064 | AT1G13120.1 Symbols: AtGLE1, GLE1: A. thaliana homolog of yeast GLE1. Chr1: 4469334-4472775 |
| | REVERSE LENGTH = 611 |
| 4065 | AT1G13195.1 Symbols: no symbol available: no full name available. Chr1: 4501778-4503347 |
| | REVERSE LENGTH = 260 |
| 4066 | AT1G13370.1 Symbols: no symbol available: no full name available. Chr1: 4587926-4588522 |
| | REVERSE LENGTH = 136 |
| 4067 | AT1G13460.1 Symbols: no symbol available: no full name available. Chr1: 4616504-4618180 |
| | FORWARD LENGTH = 492 |
| 4068 | AT1G13640.1 Symbols: no symbol available: no full name available. Chr1: 4677276-4679144 |
| | REVERSE LENGTH = 622 |
| 4069 | AT1G13860.4 Symbols: QUL1: QUASIMODO2 LIKE 1. Chr1: 4743754-4746256 REVERSE |
| | LENGTH = 603 |
| 4070 | AT1G13910.1 Symbols: no symbol available: no full name available. Chr1: 4755955-4757814 |
| | FORWARD LENGTH = 330 |
| 4071 | AT1G13970.1 Symbols: no symbol available: no full name available. Chr1: 4779742-4781882 |
| | REVERSE LENGTH = 504 |
| 4072 | AT1G14020.1 Symbols: no symbol available: no full name available. Chr1: 4802930-4805111 |
| | FORWARD LENGTH = 499 |
| 4073 | AT1G14180.1 Symbols: no symbol available: no full name available. Chr1: 4847828-4848970 |
| | FORWARD LENGTH = 348 |
| 4074 | AT1G14270.1 Symbols: no symbol available: no full name available. Chr1: 4875099-4877195 |
| | REVERSE LENGTH = 353 |
| 4075 | AT1G14560.1 Symbols: CoAc1: CoA Carrier 1. Chr1: 4981300-4983082 FORWARD LENGTH = 331 |
| 4076 | AT1G14590.1 Symbols: no symbol available: no full name available. Chr1: 4998957-5000617 |
| | REVERSE LENGTH = 386 |
| 4077 | AT1G14685.3 Symbols: ATBPC2, BBR/BPC2, BPC2: BASIC PENTACYSTEINE 2, basic pentacysteine |
| | 2. Chr1: 5043086-5043925 FORWARD LENGTH = 279 |
| 4078 | AT1G14820.3 Symbols: no symbol available: no full name available. Chr1: 5105237-5106793 |
| | REVERSE LENGTH = 252 |
| 4079 | AT1G15490.1 Symbols: no symbol available: no full name available. Chr1: 5320859-5323422 |
| | REVERSE LENGTH = 648 |
| 4080 | AT1G15750.1 Symbols: TPL, WSIP1: WUS-INTERACTING PROTEIN 1, TOPLESS. Chr1: 5415086- |
| | 5420359 REVERSE LENGTH = 1131 |
| 4081 | AT1G15920.2 Symbols: no symbol available: no full name available. Chr1: 5469962-5470822 |
| | FORWARD LENGTH = 286 |
| 4082 | AT1G15970.1 Symbols: no symbol available: no full name available. Chr1: 5486544-5488494 |
| | REVERSE LENGTH = 352 |
| 4083 | AT1G16060.1 Symbols: ADAP, WRI3: ARIA-interacting double AP2 domain protein, WRINKLED 3. |
| | Chr1: 5508563-5511609 FORWARD LENGTH = 345 |
| 4084 | AT1G16250.1 Symbols: no symbol available: no full name available. Chr1: 5557214-5558697 |
| | FORWARD LENGTH = 383 |
| 4085 | AT1G17040.2 Symbols: SHA, ATSHA, STATLA: ARABIDOPSIS THALIANA SH2 DOMAIN |
| | PROTEIN A, SH2 domain protein A, STAT-TYPE LINKER-SH2 DOMAIN FACTOR A. Chr1: 5825109- |
| | 5828056 FORWARD LENGTH = 651 |
| 4086 | AT1G17680.1 Symbols: no symbol available: no full name available. Chr1: 6076388-6082160 |
| | FORWARD LENGTH = 896 |
| 4087 | AT1G17720.1 Symbols: ATB BETA.: Chr1: 6093949-6098065 REVERSE LENGTH = 501 |
| 4088 | AT1G17780.2 Symbols: no symbol available: no full name available. Chr1: 6124095-6125166 |
| | FORWARD LENGTH = 263 |
| 4089 | AT1G17890.1 Symbols: GER2.: Chr1: 6154478-6155596 REVERSE LENGTH = 328 |
| 4090 | AT1G17920.1 Symbols: HDG12: homeodomain GLABROUS 12. Chr1: 6162214-6165033 REVERSE |
| | LENGTH = 687 |
| 4091 | AT1G17980.1 Symbols: PAPS1: poly(A) polymerase 1. Chr1: 6187742-6191418 REVERSE |
| | LENGTH = 713 |
| 4092 | AT1G17990.1 Symbols: no symbol available: no full name available. Chr1: 6192455-6193755 |
| | REVERSE LENGTH = 269 |
| 4093 | AT1G18160.1 Symbols: no symbol available: no full name available. Chr1: 6249126-6253835 |
| | FORWARD LENGTH = 992 |
| 4094 | AT1G18330.2 Symbols: EPR1, RVE7: EARLY-PHYTOCHROME-RESPONSIVEI, REVEILLE 7. |
| | Chr1: 6306196-6307718 REVERSE LENGTH = 372 |
| 4095 | AT1G18570.1 Symbols: HIG1, MYB51, AtMYB51, BW51A, BW51B: myb domain protein 51, HIGH |
| | INDOLIC GLUCOSINOLATE 1. Chr1: 6389765-6391026 FORWARD LENGTH = 352 |
| 4096 | AT1G18690.1 Symbols: XXT4: xyloglucan xylosyltransferase 4. Chr1: 6435153-6436694 FORWARD |
| | LENGTH = 513 |
| 4097 | AT1G18750.1 Symbols: AGL65: AGAMOUS-like 65. Chr1: 6467266-6469640 FORWARD |
| | LENGTH = 389 |
| 4098 | AT1G18840.1 Symbols: IQD30: IQ-domain 30. Chr1: 6501068-6503435 REVERSE LENGTH = 572 |
| 4099 | AT1G19220.1 Symbols: IAA22, ARF19, ARF11: AUXIN RESPONSE FACTOR11, auxin response factor |
| | 19, indole-3-acetic acid inducible 22. Chr1: 6628395-6632779 REVERSE LENGTH = 1086 |
| 4100 | AT1G19330.3 Symbols: no symbol available: no full name available. Chrl: 6680824-6683290 |
| | REVERSE LENGTH = 248 |
| 4101 | AT1G19370.1 Symbols: no symbol available: no full name available. Chr1: 6692904-6694721 |
| | REVERSE LENGTH = 605 |
| 4102 | AT1G19396.2 Symbols: no symbol available: no full name available. Chr1: 6709873-6710223 |
| | FORWARD LENGTH = 64 |
| 4103 | AT1G19400.1 Symbols: no symbol available: no full name available. Chr1: 6712222-6713676 |
| | REVERSE LENGTH = 303 |
| 4104 | AT1G19450.1 Symbols: no symbol available: no full name available. Chr1: 6731671-6734633 |
| | REVERSE LENGTH = 488 |
| 4105 | AT1G19700.1 Symbols: BLH10, BEL10: BEL1-like homeodomain 10, BEL1-LIKE HOMEODOMAIN |
| | 10. Chr1: 6809958-6811854 REVERSE LENGTH = 538 |
| 4106 | AT1G19800.2 Symbols: TGD1, ABCI14: ATP-binding cassette 114, trigalactosyldiacylglycerol 1. |
| | Chr1: 6846812-6847954 FORWARD LENGTH = 350 |
| 4107 | AT1G20550.1 Symbols: no symbol available: no full name available. Chr1: 7115485-7117936 |
| | REVERSE LENGTH = 564 |
| 4108 | AT1G20650.1 Symbols: ASG5: ALTERED SEED GERMINATION 5. Chr1: 7158422-7160022 |
| | REVERSE LENGTH = 381 |
| 4109 | AT1G20880.1 Symbols: no symbol available: no full name available. Chr1: 7262879-7264834 |
| | REVERSE LENGTH = 274 |
| 4110 | AT1G21160.1 Symbols: no symbol available: no full name available. Chr1: 7408121-7412455 |
| | REVERSE LENGTH = 1092 |
| 4111 | AT1G21350.3 Symbols: no symbol available: no full name available. Chr1: 7477376-7479012 |
| | REVERSE LENGTH = 252 |
| 4112 | AT1G21560.2 Symbols: no symbol available: no full name available. Chr1: 7555208-7556789 |
| | FORWARD LENGTH = 399 |
| 4113 | AT1G21780.1 Symbols: no symbol available: no full name available. Chr1: 7652476-7653866 |
| | FORWARD LENGTH = 326 |
| 4114 | AT1G21840.1 Symbols: UREF: urease accessory protein F. Chr1: 7666859-7667581 FORWARD |
| | LENGTH = 240 |
| 4115 | AT1G21930.1 Symbols: no symbol available: no full name available. Chr1: 7713466-7714298 |
| | FORWARD LENGTH = 98 |
| 4116 | AT1G22050.1 Symbols: MUB6: membrane-anchored ubiquitin-fold protein 6 precursor. Chr1: 7771897- |
| | 7772843 FORWARD LENGTH = 119 |
| 4117 | AT1G22180.2 Symbols: no symbol available: no full name available. Chr1: 7828434-7829745 |
| | REVERSE LENGTH = 314 |
| 4118 | AT1G22190.1 Symbols: RAP2.4, WIND2: related to AP2 4. Chr1: 7836245-7837030 FORWARD |
| | LENGTH = 261 |
| 4119 | AT1G22520.2 Symbols: no symbol available: no full name available. Chr1: 7952996-7954304 |
| | REVERSE LENGTH = 179 |
| 4120 | AT1G23150.1 Symbols: no symbol available: no full name available. Chr1: 8206948-8207461 |
| | FORWARD LENGTH = 141 |
| 4121 | AT1G23360.1 Symbols: MENG.: Chr1: 8295421-8296893 REVERSE LENGTH = 261 |
| 4122 | AT1G23480.1 Symbols: ATCSLA3, CSLA03, CSLA3, ATCSLA03: CELLULOSE SYNTHASE-LIKE |
| | A3, cellulose synthase-like A3. Chr1: 8333917-8336230 FORWARD LENGTH = 556 |
| 4123 | AT1G23730.1 Symbols: ATBCA3, BCA3: beta carbonic anhydrase 3, BETA CARBONIC ANHYDRASE |
| | 3. Chr1: 8395965-8398014 FORWARD LENGTH = 258 |
| 4124 | AT1G24030.1 Symbols: no symbol available: no full name available. Chr1: 8503394-8505195 |
| | FORWARD LENGTH = 375 |
| 4125 | AT1G24510.1 Symbols: no symbol available: no full name available. Chr1: 8685504-8688101 |
| | REVERSE LENGTH = 535 |
| 4126 | AT1G25280.1 Symbols: AtTLP10, TLP10: tubby like protein 10. Chr1: 8864961-8866608 FORWARD |
| | LENGTH = 445 |
| 4127 | AT1G25420.1 Symbols: ISTL2: IST1-LIKE 2. Chr1: 8916141-8917980 FORWARD LENGTH = 323 |
| 4128 | AT1G25470.1 Symbols: CRF12: cytokinin response factor 12. Chr1: 8944901-8945764 REVERSE |
| | LENGTH = 287 |
| 4129 | AT1G25500.2 Symbols: no symbol available: no full name available. Chr1: 8955437-8957793 |
| | REVERSE LENGTH = 488 |
| 4130 | AT1G26150.1 Symbols: PERK10, AtPERK10: proline-rich extensin-like receptor kinase 10. |
| | Chr1: 9039790-9042873 REVERSE LENGTH = 762 |
| 4131 | AT1G26230.1 Symbols: Cpn60beta4: chaperonin-60beta4. Chr1: 9072388-9075272 REVERSE |
| | LENGTH = 611 |
| 4132 | AT1G26440.3 Symbols: ATUPS5, UPS5: ureide permease 5. Chr1: 9144109-9145673 REVERSE |
| | LENGTH = 413 |
| 4133 | AT1G26520.1 Symbols: no symbol available: no full name available. Chr1: 9163448-9165594 |
| | REVERSE LENGTH = 374 |
| 4134 | AT1G26530.1 Symbols: no symbol available: no full name available. Chr1: 9165890-9167298 |
| | FORWARD LENGTH = 178 |
| 4135 | AT1G26660.2 Symbols: no symbol available: no full name available. Chr1: 9212298-9213765 |
| | FORWARD LENGTH = 177 |
| 4136 | AT1G26850.1 Symbols: no symbol available: no full name available. Chr1: 9301146-9303432 |
| | REVERSE LENGTH = 616 |
| 4137 | AT1G27460.1 Symbols: NPGRI: no pollen germination related 1. Chr1: 9534977-9537424 FORWARD |
| | LENGTH = 694 |
| 4138 | AT1G27760.3 Symbols: ATSAT32, SAT32: SALT-TOLERANCE 32. Chr1: 9668895-9671322 |
| | FORWARD LENGTH = 441 |
| 4139 | AT1G27840.3 Symbols: ATCSA-1.: Chr1: 9693332-9696257 REVERSE LENGTH = 451 |
| 4140 | AT1G27850.1 Symbols: no symbol available: no full name available. Chr1: 9699265-9703701 |
| | FORWARD LENGTH = 1148 |
| 4141 | AT1G28090.1 Symbols: no symbol available: no full name available. Chr1: 9795826-9798891 |
| | FORWARD LENGTH = 541 |
| 4142 | AT1G28135.1 Symbols: no symbol available: no full name available. Chr1: 9829332-9829487 |
| | FORWARD LENGTH = 51 |
| 4143 | AT1G28260.1 Symbols: no symbol available: no full name available. Chr1: 9875776-9878782 |
| | REVERSE LENGTH = 880 |
| 4144 | AT1G28560.2 Symbols: SRD2: SHOOT REDIFFERENTIATION DEFECTIVE 2. Chr1: 10038197- |
| | 10040382 REVERSE LENGTH = 386 |
| 4145 | AT1G28580.1 Symbols: no symbol available: no full name available. Chr1: 10044603-10046379 |
| | REVERSE LENGTH = 390 |
| 4146 | AT1G28710.1 Symbols: no symbol available: no full name available. Chr1: 10086850-10088025 |
| | REVERSE LENGTH = 340 |
| 4147 | AT1G29120.5 Symbols: UP9: UP9. Chr1: 10174481-10178143 FORWARD LENGTH = 456 |
| 4148 | AT1G29390.1 Symbols: COR314-TM2, COR413IM2: cold regulated 314 thylakoid membrane 2, COLD |
| | REGULATED 314 INNER MEMBRANE 2. Chr1: 10286409-10287878 REVERSE LENGTH = 226 |
| 4149 | AT1G29465.1 Symbols: no symbol available: no full name available. Chr1: 10309626-10309736 |
| | FORWARD LENGTH = 36 |
| 4150 | AT1G29890.2 Symbols: RWA4: REDUCED WALL ACETYLATION 4. Chr1: 10463532-10467153 |
| | FORWARD LENGTH = 584 |
| 4151 | AT1G29950.1 Symbols: no symbol available: no full name available. Chr1: 10493461-10494216 |
| | FORWARD LENGTH = 251 |
| 4152 | AT1G30210.1 Symbols: ATTCP24, TCP24: TEOSINTE BRANCHED 1, cycloidea, and PCF family 24. |
| | Chr1: 10628754-10629728 REVERSE LENGTH = 324 |
| 4153 | AT1G30270.1 Symbols: SnRK3.23, PKS17, CIPK23, ATCIPK23, LKS1: SOS2-like protein kinase |
| | 17, LOW-K+-SENSITIVE 1, CBL-interacting protein kinase 23, SNF1-RELATED PROTEIN KINASE |
| | 3.23. Chr1: 10655270-10658524 FORWARD LENGTH = 482 |
| 4154 | AT1G30330.2 Symbols: ARF6: auxin response factor 6. Chr1: 10686125-10690036 REVERSE |
| | LENGTH = 935 |
| 4155 | AT1G30650.1 Symbols: AR411, WRKY14, ATWRKY14: WRKY DNA-BINDING PROTEIN 14, WRKY |
| | DNA-binding protein 14. Chr1: 10868612-10870973 FORWARD LENGTH = 430 |
| 4156 | AT1G30820.1 Symbols: no symbol available: no full name available. Chr1: 10945251-10948825 |
| | REVERSE LENGTH = 600 |
| 4157 | AT1G31300.1 Symbols: no symbol available: no full name available. Chr1: 11194308-11195955 |
| | FORWARD LENGTH = 278 |
| 4158 | AT1G31460.1 Symbols: no symbol available: no full name available. Chr1: 11261705-11262610 |
| | REVERSE LENGTH = 301 |
| 4159 | AT1G31600.1 Symbols: TRM9, AtTRM9: tRNA methyltransferase 9, Arabidopsis thaliana tRNA |
| | methyltransferase 9. Chr1: 11313376-11315199 REVERSE LENGTH = 431 |
| 4160 | AT1G31880.1 Symbols: NLM9, BRX: BREVIS RADIX. Chr1: 11447804-11450053 FORWARD |
| | LENGTH = 344 |
| 4161 | AT1G31910.1 Symbols: no symbol available: no full name available. Chr1: 11459050-11461649 |
| | FORWARD LENGTH = 505 |
| 4162 | AT1G32230.1 Symbols: RIMB1, ATP8, RCD1, CEO, CEO1, AtRCD1: RADICAL-INDUCED CELL |
| | DEATH1, ARABIDOPSIS THALIANA P8 (INTERACTING PROTEIN), REDOX IMBALANCED 1. |
| | Chr1: 11613427-11615894 FORWARD LENGTH = 589 |
| 4163 | AT1G32540.1 Symbols: LOL1: lsd one like 1. Chr1: 11768253-11769591 FORWARD LENGTH = 187 |
| 4164 | AT1G32750.1 Symbols: GTD1, HAF01, HAF1, HAC13, TAF1: TBP-ASSOCIATED FACTOR |
| | 1, HISTONE ACETYLTRANSFERASE OF THE CBP FAMILY 13, HISTONE |
| | ACETYLTRANSFERASE OF THE TAFII250 FAMILY 1. Chr1: 11846385-11856261 REVERSE |
| | LENGTH = 1919 |
| 4165 | AT1G32780.1 Symbols: no symbol available: no full name available. Chr1: 11869977-11872595 |
| | REVERSE LENGTH = 394 |
| 4166 | AT1G34190.1 Symbols: anac017, NAC017, RAO2: NAC domain containing protein 17, regulators of |
| | aoxla 2. Chr1: 12451729-12453914 FORWARD LENGTH = 557 |
| 4167 | AT1G35340.1 Symbols: no symbol available: no full name available. Chr1: 12977768-12979749 |
| | FORWARD LENGTH = 316 |
| 4168 | AT1G43690.1 Symbols: no symbol available: no full name available. Chr1: 16478519-16482589 |
| | FORWARD LENGTH = 599 |
| 4169 | AT1G44750.1 Symbols: PUP11, ATPUP11: purine permease 11. Chr1: 16892688-16895168 FORWARD |
| | LENGTH = 379 |
| 4170 | AT1G44890.1 Symbols: no symbol available: no full name available. Chr1: 16963358-16964818 |
| | FORWARD LENGTH = 281 |
| 4171 | AT1G47740.1 Symbols: no symbol available: no full name available. Chr1: 17567903-17569035 |
| | FORWARD LENGTH = 279 |
| 4172 | AT1G48140.1 Symbols: DPMS3: dolichol phosphate mannose synthase 3. Chr1: 17783402-17783914 |
| | FORWARD LENGTH = 89 |
| 4173 | AT1G48330.1 Symbols: no symbol available: no full name available. Chr1: 17863897-17864124 |
| | FORWARD LENGTH = 75 |
| 4174 | AT1G48635.2 Symbols: PEX3-2, PEX3: PEROXIN 3-2, peroxin 3. Chr1: 17983951-17985966 |
| | FORWARD LENGTH = 400 |
| 4175 | AT1G49000.1 Symbols: no symbol available: no full name available. Chr1: 18123559-18124029 |
| | REVERSE LENGTH = 156 |
| 4176 | AT1G49130.1 Symbols: BBX17: B-box domain protein 17. Chr1: 18174741-18175936 REVERSE |
| | LENGTH = 326 |
| 4177 | AT1G50360.1 Symbols: ATVIIIA, VIIIA: MYOSIN VIII A. Chr1: 18650688-18657106 FORWARD |
| | LENGTH = 1153 |
| 4178 | AT1G50440.1 Symbols: no symbol available: no full name available. Chr1: 18686099-18687646 |
| | FORWARD LENGTH = 250 |
| 4179 | AT1G50460.1 Symbols: HKL1, ATHKL1: hexokinase-like 1. Chr1: 18694031-18697429 FORWARD |
| | LENGTH = 498 |
| 4180 | AT1G51745.1 Symbols: SL1.: Chr1: 19192055-19194479 FORWARD LENGTH = 635 |
| 4181 | AT1G52080.1 Symbols: AR791.: Chr1: 19369788-19371862 FORWARD LENGTH = 573 |
| 4182 | AT1G52100.1 Symbols: no symbol available: no full name available. Chr1: 19383291-19385123 |
| | REVERSE LENGTH = 437 |
| 4183 | AT1G52150.2 Symbols: ATHB15, CNA, ICU4, ATHB-15: INCURVATA 4, CORONA. Chr1: 19409913- |
| | 19413961 REVERSE LENGTH = 837 |
| 4184 | AT1G52370.1 Symbols: no symbol available: no full name available. Chr1: 19507052-19508699 |
| | FORWARD LENGTH = 269 |
| 4185 | AT1G52630.1 Symbols: no symbol available: no full name available. Chr1: 19606470-19608526 |
| | REVERSE LENGTH = 439 |
| 4186 | AT1G53035.1 Symbols: no symbol available: no full name available. Chr1: 19761866-19762318 |
| | REVERSE LENGTH = 150 |
| 4187 | AT1G53110.1 Symbols: no symbol available: no full name available. Chr1: 19790434-19792016 |
| | FORWARD LENGTH = 439 |
| 4188 | AT1G53200.1 Symbols: no symbol available: no full name available. Chrl: 19840486-19843169 |
| | REVERSE LENGTH = 613 |
| 4189 | AT1G53300.1 Symbols: TTL1: tetratricopeptide-repeat thioredoxin-like 1. Chr1: 19879726-19882375 |
| | FORWARD LENGTH = 699 |
| 4190 | AT1G53430.1 Symbols: no symbol available: no full name available. Chr1: 19935298-19940959 |
| | FORWARD LENGTH = 1038 |
| 4191 | AT1G53490.1 Symbols: HEI10; homolog of human HEI10 ( Enhancer of cell Invasion No. 10). |
| | Chr1: 19965146-19966811 FORWARD LENGTH = 304 |
| 4192 | AT1G53650.1 Symbols: CID8: CTC-interacting domain 8. Chr1: 20029262-20031243 REVERSE |
| | LENGTH = 314 |
| 4193 | AT1G53780.2 Symbols: no symbol available: no full name available. Chr1: 20074212-20077713 |
| | REVERSE LENGTH = 620 |
| 4194 | AT1G54180.1 Symbols: BRX-LIKE3, ATBRXL3: BREVIS RADIX-like 3, ARABIDOPSIS THALIANA |
| | BREVIS RADIX-LIKE 3. Chr1: 20227697-20229728 FORWARD LENGTH = 370 |
| 4195 | AT1G54360.2 Symbols: TAF6B, TAF6B1, TAF6B2, TAF6B4: TBP-ASSOCIATED FACTOR 6B2, TBP- |
| | ASSOCIATED FACTOR 6B1, TBP-ASSOCIATED FACTOR 6B, TBP-associated factor 6B4. |
| | Chr1: 20290600-20293160 FORWARD LENGTH = 527 |
| 4196 | AT1G54390.2 Symbols: ING2: INHIBITOR OF GROWTH 2. Chr1: 20304833-20306943 REVERSE |
| | LENGTH = 328 |
| 4197 | AT1G54410.1 Symbols: HIRD11, AtHIRD11: dehydrin 11 kDa. Chr1: 20310305-20310601 REVERSE |
| | LENGTH = 98 |
| 4198 | AT1G55120.1 Symbols: FRUCT5, AtcwINV3, ATFRUCT5: beta-fructofuranosidase 5,6-fructan |
| | exohydrolase. Chr1: 20566617-20569165 FORWARD LENGTH = 594 |
| 4199 | AT1G55350.5 Symbols: EMB80, EMB1275, ATDEK1, DEK1: EMBRYO DEFECTIVE 80, DEFECTIVE |
| | KERNEL 1, embryo defective 1275. Chr1: 20654463-20664501 REVERSE LENGTH = 2179 |
| 4200 | AT1G55500.2 Symbols: ECT4: evolutionarily conserved C-terminal region 4. Chr1: 20719747-20722416 |
| | FORWARD LENGTH = 599 |
| 4201 | AT1G55580.1 Symbols: SCL18, LAS: Lateral Suppressor, SCARECROW-LIKE 18. Chr1: 20764106- |
| | 20765443 FORWARD LENGTH = 445 |
| 4202 | AT1G55610.1 Symbols: BRL1: BRI1 like. Chr1: 20779874-20783374 REVERSE LENGTH = 1166 |
| 4203 | AT1G55630.1 Symbols: no symbol available: no full name available. Chr1: 20791817-20793250 |
| | REVERSE LENGTH = 477 |
| 4204 | AT1G55675.1 Symbols: no symbol available: no full name available. Chr1: 20803854-20804180 |
| | REVERSE LENGTH = 108 |
| 4205 | AT1G55680.1 Symbols: no symbol available: no full name available. Chr1: 20804988-20807294 |
| | REVERSE LENGTH = 445 |
| 4206 | AT1G55760.1 Symbols: no symbol available: no full name available. Chr1: 20847117-20848507 |
| | REVERSE LENGTH = 329 |
| 4207 | AT1G56045.1 Symbols: no symbol available: no full name available. Chr1: 20962686-20963268 |
| | FORWARD LENGTH = 25 |
| 4208 | AT1G57610.1 Symbols: no symbol available: no full name available. Chr1: 21337449-21338412 |
| | REVERSE LENGTH = 293 |
| 4209 | AT1G57700.1 Symbols: no symbol available: no full name available. Chr1: 21371051-21373860 |
| | FORWARD LENGTH = 692 |
| 4210 | AT1G58180.2 Symbols: BCA6, ATBCA6: A. THALIANA BETA CARBONIC ANHYDRASE 6, beta |
| | carbonic anhydrase 6. Chr1: 21538010-21539711 REVERSE LENGTH = 290 |
| 4211 | AT1G58200.1 Symbols: MSL3: MSCS-like 3. Chr1: 21548370-21552488 REVERSE LENGTH = 678 |
| 4212 | AT1G58250.2 Symbols: HPS4, SAB: hypersensitive to Pi starvation 4, SABRE. Chr1: 21587317- |
| | 21601373 REVERSE LENGTH = 2655 |
| 4213 | AT1G58350.1 Symbols: ZW18.: Chr1: 21663131-21666827 REVERSE LENGTH = 794 |
| 4214 | AT1G58602.1 Symbols: no symbol available: no full name available. Chr1: 21760167-21763765 |
| | FORWARD LENGTH = 1138 |
| 4215 | AT1G60440.1 Symbols: PANK1, ATPANK1, ATCOAA: pantothenate kinase 1. Chr1: 22266653- |
| | 22269070 REVERSE LENGTH = 383 |
| 4216 | AT1G60850.2 Symbols: AAC42, ATRPAC42.: Chr1: 22398078-22400155 REVERSE LENGTH = 375 |
| 4217 | AT1G60890.2 Symbols: no symbol available: no full name available. Chr1: 22412452-22416498 |
| | REVERSE LENGTH = 781 |
| 4218 | AT1G61150.1 Symbols: no symbol available: no full name available. Chr1: 22542928-22544355 |
| | FORWARD LENGTH = 243 |
| 4219 | AT1G61410.1 Symbols: no symbol available: no full name available. Chr1: 22658708-22659064 |
| | REVERSE LENGTH = 118 |
| 4220 | AT1G61520.1 Symbols: LHCA3: photosystem I light harvesting complex gene 3. Chr1: 22700152- |
| | 22701149 FORWARD LENGTH = 273 |
| 4221 | AT1G61820.1 Symbols: BGLU46: beta glucosidase 46. Chr1: 22835452-22838444 FORWARD |
| | LENGTH = 516 |
| 4222 | AT1G62305.1 Symbols: no symbol available: no full name available. Chr1: 23026989-23029189 |
| | REVERSE LENGTH = 378 |
| 4223 | AT1G62540.1 Symbols: FMO GS-OX2: flavin-monooxygenase glucosinolate S-oxygenase 2. |
| | Chr1: 23151870-23155427 FORWARD LENGTH = 457 |
| 4224 | AT1G62610.4 Symbols: no symbol available: no full name available. Chr1: 23181531-23182454 |
| | REVERSE LENGTH = 282 |
| 4225 | AT1G62840.1 Symbols: no symbol available: no full name available. Chr1: 23271481-23272407 |
| | FORWARD LENGTH = 224 |
| 4226 | AT1G63010.5 Symbols: VPT1, PHT5_1: Vacuolar Phosphate Transporter 1. Chr1: 23347972-23351026 |
| | REVERSE LENGTH = 708 |
| 4227 | AT1G63480.1 Symbols: AHL12: AT-hook motif nuclear localized protein 12. Chr1: 23539872-23541685 |
| | REVERSE LENGTH = 361 |
| 4228 | AT1G63670.1 Symbols: TRM12: TON1 Recruiting Motif 12. Chr1: 23607527-23610003 REVERSE |
| | LENGTH = 689 |
| 4229 | AT1G64110.2 Symbols: DAA1: DUO1-activated ATPase 1. Chr1: 23796887-23801255 REVERSE |
| | LENGTH = 829 |
| 4230 | AT1G64140.1 Symbols: no symbol available: no full name available. Chr1: 23804069-23806009 |
| | REVERSE LENGTH = 646 |
| 4231 | AT1G64230.4 Symbols: UBC28: ubiquitin-conjugating enzyme 28. Chr1: 23833792-23835220 |
| | FORWARD LENGTH = 190 |
| 4232 | AT1G64630.1 Symbols: WNK10, ATWNK10: with no lysine (K) kinase 10, WITH NO LYSINE KINASE |
| | 10. Chr1: 24019920-24022114 FORWARD LENGTH = 524 |
| 4233 | AT1G64990.1 Symbols: GTG1: GPCR-type G protein 1. Chr1: 24140183-24145866 REVERSE |
| | LENGTH = 468 |
| 4234 | AT1G65070.2 Symbols: no symbol available: no full name available. Chr1: 24173047-24176244 |
| | REVERSE LENGTH = 876 |
| 4235 | AT1G65840.1 Symbols: PAO4, ATPAO4: polyamine oxidase 4. Chr1: 24490173-24492728 FORWARD |
| | LENGTH = 497 |
| 4236 | AT1G66660.2 Symbols: no symbol available: no full name available. Chr1: 24862056-24863332 |
| | REVERSE LENGTH = 348 |
| 4237 | AT1G67325.2 Symbols: no symbol available: no full name available. Chr1: 25209825-25212412 |
| | REVERSE LENGTH = 288 |
| 4238 | AT1G67480.1 Symbols: no symbol available: no full name available. Chr1: 25277294-25278529 |
| | FORWARD LENGTH = 376 |
| 4239 | AT1G67570.1 Symbols: no symbol available: no full name available. Chr1: 25325318-25326938 |
| | FORWARD LENGTH = 456 |
| 4240 | AT1G67800.2 Symbols: no symbol available: no full name available. Chr1: 25421029-25423297 |
| | REVERSE LENGTH = 453 |
| 4241 | AT1G67900.1 Symbols: no symbol available: no full name available. Chr1: 25467737-25469888 |
| | FORWARD LENGTH = 631 |
| 4242 | AT1G68130.1 Symbols: IDD14alpha, ALIDD14, IDD14beta, IDD14: indeterminate(ID)-domain 14. |
| | Chr1: 25532484-25534317 FORWARD LENGTH = 419 |
| 4243 | AT1G68160.1 Symbols: no symbol available: no full name available. Chr1: 25546168-25548625 |
| | REVERSE LENGTH = 273 |
| 4244 | AT1G68410.1 Symbols: no symbol available: no full name available. Chr1: 25650262-25652255 |
| | REVERSE LENGTH = 436 |
| 4245 | AT1G68550.1 Symbols: CRF10: cytokinin response factor 10. Chr1: 25725810-25726784 REVERSE |
| | LENGTH = 324 |
| 4246 | AT1G68920.1 Symbols: bHLH49, CIL1: CIB1 Like protein 1. Chr1: 25915620-25917675 FORWARD |
| | LENGTH = 486 |
| 4247 | AT1G69170.1 Symbols: AtSPL6, SPL6: SQUAMOSA PROMOTER BINDING PROTEIN (SBP)-domain |
| | transcription factor 6. Chr1: 26005626-26007041 FORWARD LENGTH = 405 |
| 4248 | AT1G69690.1 Symbols: AtTCP15, TCP15: TEOSINTE BRANCHED1/CYCLOIDEA/PCF 15. |
| | Chr1: 26216449-26217426 FORWARD LENGTH = 325 |
| 4249 | AT1G69750.1 Symbols: ATCOX19-2, COX19-2: cytochrome c oxidase 19-2, A. THALIANA |
| | CYTOCHROME C OXIDASE 19-2. Chr1: 26236352-26237316 FORWARD LENGTH = 98 |
| 4250 | AT1G69840.1 Symbols: no symbol available: no full name available. Chr1: 26293932-26295150 |
| | REVERSE LENGTH = 286 |
| 4251 | AT1G69870.1 Symbols: NRT1.7, AtNPF2.13, NPF2.13: nitrate transporter 1.7, NRT1/PTR family 2.13. |
| | Chr1: 26316208-26320097 FORWARD LENGTH = 620 |
| 4252 | AT1G70160.1 Symbols: no symbol available: no full name available. Chr1: 26420159-26422345 |
| | FORWARD LENGTH = 523 |
| 4253 | AT1G70280.2 Symbols: no symbol available: no full name available. Chr1: 26466086-26468471 |
| | REVERSE LENGTH = 509 |
| 4254 | AT1G70340.1 Symbols: no symbol available: no full name available. Chr1: 26504567-26506389 |
| | REVERSE LENGTH = 510 |
| 4255 | AT1G70670.1 Symbols: CLO4, AtCLO4, PXG4: peroxygenase 4, Arabidopsis thaliana caleosin 4, caleosin |
| | 4. Chr1: 26644830-26645970 FORWARD LENGTH = 195 |
| 4256 | AT1G70780.1 Symbols: no symbol available: no full name available. Chr1: 26695462-26695975 |
| | REVERSE LENGTH = 140 |
| 4257 | AT1G71020.1 Symbols: PUB10: PLANT U-BOX PROTEIN10. Chr1: 26790825-26793105 REVERSE |
| | LENGTH = 628 |
| 4258 | AT1G71340.1 Symbols: AtGDPD4, GDPD4: glycerophosphodiester phosphodiesterase 4. |
| | Chr1: 26885556-26887321 REVERSE LENGTH = 328 |
| 4259 | AT1G71696.2 Symbols: SOL1: SUPPRESSOR OF LLP1 1. Chr1: 26966996-26970364 FORWARD |
| | LENGTH = 491 |
| 4260 | AT1G71940.2 Symbols: no symbol available: no full name available. Chr1: 27078781-27080191 |
| | FORWARD LENGTH = 306 |
| 4261 | AT1G71980.1 Symbols: no symbol available: no full name available. Chr1: 27098250-27099881 |
| | FORWARD LENGTH = 448 |
| 4262 | AT1G72130.1 Symbols: no symbol available: no full name available. Chr1: 27137201-27139223 |
| | FORWARD LENGTH = 538 |
| 4263 | AT1G72510.1 Symbols: no symbol available: no full name available. Chr1: 27303906-27304403 |
| | FORWARD LENGTH = 165 |
| 4264 | AT1G72770.1 Symbols: HAB1, AtHAB1: HYPERSENSITIVE TO ABA1. Chr1: 27390998-27392851 |
| | FORWARD LENGTH = 511 |
| 4265 | AT1G72820.1 Symbols: no symbol available: no full name available. Chr1: 27403457-27404506 |
| | FORWARD LENGTH = 349 |
| 4266 | AT1G72830.2 Symbols: ATHAP2C, NF-YA3, HAP2C: “nuclear factor Y, subunit A3”. Chr1: 27405699- |
| | 27407088 REVERSE LENGTH = 341 |
| 4267 | AT1G72990.1 Symbols: BGAL17: beta-galactosidase 17. Chr1: 27457480-27462168 REVERSE |
| | LENGTH = 697 |
| 4268 | AT1G73360.1 Symbols: EDT1, ATHDG11, HDG11, AtEDT1: homeodomain GLABROUS |
| | 11, HOMEODOMAIN GLABROUS 11, ENHANCED DROUGHT TOLERANCE 1. Chr1: 27578893- |
| | 27581820 REVERSE LENGTH = 722 |
| 4269 | AT1G73370.1 Symbols: ATSUS6, SUS6: ARABIDOPSIS THALIANA SUCROSE SYNTHASE |
| | 6, sucrose synthase 6. Chr1: 27584533-27588326 REVERSE LENGTH = 942 |
| 4270 | AT1G73470.3 Symbols: no symbol available: no full name available. Chr1: 27625208-27627604 |
| | FORWARD LENGTH = 357 |
| 4271 | AT1G73480.1 Symbols: MAGL4.: Chr1: 27629266-27632486 FORWARD LENGTH = 463 |
| 4272 | AT1G74160.1 Symbols: TRM4: TONI Recruiting Motif 4. Chr1: 27887164-27891151 FORWARD |
| | LENGTH = 1025 |
| 4273 | AT1G74370.1 Symbols: no symbol available: no full name available. Chr1: 27958200-27958985 |
| | REVERSE LENGTH = 261 |
| 4274 | AT1G74650.1 Symbols: ATY13, MYB31, ATMYB31: ARABIDOPSIS THALIANA MYB DOMAIN |
| | PROTEIN 31, myb domain protein 31. Chr1: 28041493-28042773 FORWARD LENGTH = 330 |
| 4275 | AT1G74680.1 Symbols: no symbol available: no full name available. Chr1: 28059528-28060984 |
| | FORWARD LENGTH = 461 |
| 4276 | AT1G74910.1 Symbols: KJC1: KONJAC 1. Chr1: 28135770-28138456 REVERSE LENGTH = 415 |
| 4277 | AT1G75190.1 Symbols: no symbol available: no full name available. Chr1: 28219598-28219993 |
| | FORWARD LENGTH = 131 |
| 4278 | AT1G75240.1 Symbols: HB33, AtHB33, ZHD5: zinc-finger homeodomain 5, homeobox protein 33. |
| | Chr1: 28241576-28242505 FORWARD LENGTH = 309 |
| 4279 | AT1G75300.1 Symbols: no symbol available: no full name available. Chr1: 28255552-28256927 |
| | FORWARD LENGTH = 322 |
| 4280 | AT1G75340.1 Symbols: no symbol available: no full name available. Chr1: 28269153-28271873 |
| | REVERSE LENGTH = 435 |
| 4281 | AT1G75390.1 Symbols: bZIP44, AtbZIP44: basic leucine-zipper 44. Chr1: 28292224-28292745 |
| | FORWARD LENGTH = 173 |
| 4282 | AT1G75450.1 Symbols: ATCKX6, ATCKX5, CKX5: ARABIDOPSIS THALIANA CYTOKININ |
| | OXIDASE 5, CYTOKININ OXIDASE 6, cytokinin oxidase 5. Chr1: 28315248-28318064 REVERSE |
| | LENGTH = 540 |
| 4283 | AT1G75600.1 Symbols: HTR14.: Chr1: 28390753-28391461 FORWARD LENGTH = 136 |
| 4284 | AT1G75730.1 Symbols: no symbol available: no full name available. Chr1: 28435991-28438983 |
| | REVERSE LENGTH = 589 |
| 4285 | AT1G76200.1 Symbols: no symbol available: no full name available. Chr1: 28593351-28593980 |
| | FORWARD LENGTH = 69 |
| 4286 | AT1G76710.1 Symbols: ASHH1, SDG26: SET domain group 26, ASH1-RELATED PROTEIN 1. |
| | Chr1: 28789887-28792371 REVERSE LENGTH = 492 |
| 4287 | AT1G76720.1 Symbols: no symbol available: no full name available. Chr1: 28794144-28798780 |
| | REVERSE LENGTH = 1191 |
| 4288 | AT1G77350.1 Symbols: no symbol available: no full name available. Chr1: 29070497-29071545 |
| | FORWARD LENGTH = 122 |
| 4289 | AT1G77460.1 Symbols: CSI3: CELLULOSE SYNTHASE INTERACTIVE 3. Chr1: 29104378- |
| | 29111580 FORWARD LENGTH = 2136 |
| 4290 | AT1G77580.2 Symbols: no symbol available: no full name available. Chr1: 29144191-29146793 |
| | REVERSE LENGTH = 779 |
| 4291 | AT1G77660.1 Symbols: no symbol available: no full name available. Chr1: 29186073-29187500 |
| | REVERSE LENGTH = 421 |
| 4292 | AT1G77680.1 Symbols: RRP44B, AtRRP44B, SOV: Rrp44 homolog B, SUPPRESSOR OF VARICOSE. |
| | Chr1: 29192188-29195963 REVERSE LENGTH = 1055 |
| 4293 | AT1G78080.1 Symbols: AtWIND1, WIND1, RAP2.4: wound induced dedifferentiation 1, related to AP2 4. |
| | Chr1: 29364790-29365794 FORWARD LENGTH = 334 |
| 4294 | AT1G78260.1 Symbols: no symbol available: no full name available. Chr1: 29447317-29450181 |
| | FORWARD LENGTH = 287 |
| 4295 | AT1G78700.1 Symbols: BEH4: BES1/BZR1 homolog 4. Chr1: 29599854-29601539 FORWARD |
| | LENGTH = 325 |
| 4296 | AT1G78710.1 Symbols: TBL42: TRICHOME BIREFRINGENCE-LIKE 42. Chr1: 29602708-29604557 |
| | FORWARD LENGTH = 359 |
| 4297 | AT1G78880.1 Symbols: no symbol available: no full name available. Chr1: 29653068-29654819 |
| | REVERSE LENGTH = 468 |
| 4298 | AT1G79220.1 Symbols: no symbol available: no full name available. Chr1: 29799227-29800426 |
| | FORWARD LENGTH = 399 |
| 4299 | AT1G79230.1 Symbols: ATMST1, STR1, MST1, ATRDHI, STI: ARABIDOPSIS THALIANA |
| | RHODANESE HOMOLOGUE 1, mercaptopyruvate sulfurtransferase 1, SULFURTRANSFERASE 1. |
| | Chr1: 29800824-29803679 FORWARD LENGTH = 379 |
| 4300 | AT1G79490.1 Symbols: EMB2217: embryo defective 2217. Chr1: 29900617-29903127 FORWARD |
| | LENGTH = 836 |
| 4301 | AT1G79690.1 Symbols: atnudt3, NUDT3: nudix hydrolase homolog 3. Chr1: 29985360-29990171 |
| | FORWARD LENGTH = 772 |
| 4302 | AT1G79790.1 Symbols: FHY1, AtcpFHy1: Arabidopsis thaliana chloroplast FMN hydrolase 1, flavin |
| | mononucleotide hydrolase 1. Chr1: 30016987-30018186 REVERSE LENGTH = 245 |
| 4303 | AT1G79810.1 Symbols: TED3, PEX2, ATPEX2: ARABIDOPSIS PEROXIN 2, PEROXIN 2, REVERSAL |
| | OF THE DET PHENOTYPE 3. Chr1: 30019944-30022156 FORWARD LENGTH = 333 |
| 4304 | AT1G80280.1 Symbols: no symbol available: no full name available. Chr1: 30183839-30186141 |
| | REVERSE LENGTH = 647 |
| 4305 | AT1G80420.1 Symbols: XRCC1, ATXRCC1: homolog of X-ray repair cross complementing 1. |
| | Chr1: 30235444-30237163 REVERSE LENGTH = 353 |
| 4306 | AT1G80570.2 Symbols: no symbol available: no full name available. Chr1: 30290661-30292231 |
| | FORWARD LENGTH = 480 |
| 4307 | AT1G80640.1 Symbols: no symbol available: no full name available. Chr1: 30311979-30314238 |
| | FORWARD LENGTH = 427 |
| 4308 | AT1G80780.3 Symbols: no symbol available: no full name available. Chr1: 30358517-30359583 |
| | FORWARD LENGTH = 286 |
| 4309 | AT1G80980.1 Symbols: no symbol available: no full name available. Chr1: 30422184-30423440 |
| | REVERSE LENGTH = 214 |
| 4310 | AT2G01100.1 Symbols: no symbol available: no full name available. Chr2: 82245-82988 FORWARD |
| | LENGTH = 247 |
| 4311 | AT2G01175.1 Symbols: no symbol available: no full name available. Chr2: 105572-105760 REVERSE |
| | LENGTH = 62 |
| 4312 | AT2G01350.1 Symbols: QPT: quinolinate phoshoribosyltransferase. Chr2: 165332-167209 REVERSE |
| | LENGTH = 348 |
| 4313 | AT2G01690.2 Symbols: no symbol available: no full name available. Chr2: 309144-313499 REVERSE LENGTH = 744 |
| 4314 | AT2G01730.1 Symbols: ATCPSF73-II, EDA26, CPSF73-II: cleavage and polyadenylation specificity |
| | factor 73 kDa subunit-II, embryo sac development arrest 26. Chr2: 320597-323845 FORWARD |
| | LENGTH = 613 |
| 4315 | AT2G01930.1 Symbols: ATBPC1, BBR, BPC1: BASIC PENTACYSTEINE1, basic pentacysteine1. |
| | Chr2: 427358-428209 REVERSE LENGTH = 283 |
| 4316 | AT2G02710.1 Symbols: PLPA, PLPB, PLPC, PLP: PAS/LOV protein B, PAS/LOV PROTEIN |
| | C, PAS/LOV PROTEIN A, PAS/LOV PROTEIN. Chr2: 758812-760608 REVERSE LENGTH = 399 |
| 4317 | AT2G02870.1 Symbols: no symbol available: no full name available. Chr2: 838378-839781 FORWARD |
| | LENGTH = 467 |
| 4318 | AT2G02930.1 Symbols: ATGSTF3, GST16, GSTF3: GLUTATHIONE S-TRANSFERASE 16, glutathione |
| | S-transferase F3. Chr2: 851348-852106 REVERSE LENGTH = 212 |
| 4319 | AT2G02960.5 Symbols: no symbol available: no full name available. Chr2: 862440-863980 REVERSE |
| | LENGTH = 275 |
| 4320 | AT2G02970.1 Symbols: APY6, AtAPY6: apyrase 6. Chr2: 865395-868007 REVERSE LENGTH = 555 |
| 4321 | AT2G03070.1 Symbols: MED8: mediator subunit 8. Chr2: 905978-908792 FORWARD LENGTH = 524 |
| 4322 | AT2G03340.1 Symbols: WRKY3: WRKY DNA-binding protein 3. Chr2: 1014724-1016936 REVERSE |
| | LENGTH = 513 |
| 4323 | AT2G03530.1 Symbols: UPS2, ATUPS2: ureide permease 2, ARABIDOPSIS THALIANA UREIDE |
| | PERMEASE 2. Chr2: 1071951-1073522 FORWARD LENGTH = 398 |
| 4324 | AT2G03810.1 Symbols: no symbol available: no full name available. Chr2: 1162703-1164286 |
| | FORWARD LENGTH = 439 |
| 4325 | AT2G03890.1 Symbols: UBDK GAMMA 7, ATPI4K GAMMA 7, PI4K GAMMA 7: UBIQUITIN-LIKE |
| | DOMAIN KINASE GAMMA 7, phosphoinositide 4-kinase gamma 7. Chr2: 1186199-1188151 |
| | FORWARD LENGTH = 650 |
| 4326 | AT2G04039.3 Symbols: no symbol available: no full name available. Chr2: 1333464-1334457 |
| | FORWARD LENGTH = 206 |
| 4327 | AT2G04240.1 Symbols: XERICO.: Chr2: 1461816-1462304 REVERSE LENGTH = 162 |
| 4328 | AT2G04378.2 Symbols: no symbol available: no full name available. Chr2: 1525640-1526169 |
| | REVERSE LENGTH = 146 |
| 4329 | AT2G04890.1 Symbols: SCL21: SCARECROW-like 21. Chr2: 1720575-1721816 REVERSE |
| | LENGTH = 413 |
| 4330 | AT2G05160.1 Symbols: no symbol available: no full name available. Chr2: 1859011-1860931 |
| | REVERSE LENGTH = 536 |
| 4331 | AT2G05210.1 Symbols: AtPOT1a, ATPOT1: Protection of Telomeres 1a. Chr2: 1891814-1894196 |
| | FORWARD LENGTH = 467 |
| 4332 | AT2G05630.2 Symbols: ATG8D.: Chr2: 2083211-2084775 REVERSE LENGTH = 164 |
| 4333 | AT2G05632.1 Symbols: no symbol available: no full name available. Chr2: 2087744-2087902 |
| | REVERSE LENGTH = 52 |
| 4334 | AT2G07727.1 Symbols: no symbol available: no full name available. Chr2: 3450863-3452044 |
| | FORWARD LENGTH = 393 |
| 4335 | AT2G12200.1 Symbols: no symbol available: no full name available. Chr2: 4900651-4900944 |
| | FORWARD LENGTH = 63 |
| 4336 | AT2G12280.1 Symbols: no symbol available: no full name available. Chr2: 4922324-4922596 |
| | FORWARD LENGTH = 90 |
| 4337 | AT2G13660.1 Symbols: no symbol available: no full name available. Chr2: 5692292-5692889 |
| | REVERSE LENGTH = 177 |
| 4338 | AT2G15530.4 Symbols: MBR1: MED25 BINDING RING-H2 PROTEIN 1. Chr2: 6774150-6777899 |
| | FORWARD LENGTH = 780 |
| 4339 | AT2G16400.1 Symbols: BLH7: BEL1-like homeodomain 7. Chr2: 7101490-7103200 REVERSE |
| | LENGTH = 482 |
| 4340 | AT2G16430.2 Symbols: ATPAP10, PAP10: purple acid phosphatase 10. Chr2: 7120502-7122772 |
| | REVERSE LENGTH = 468 |
| 4341 | AT2G16586.1 Symbols: no symbol available: no full name available. Chr2: 7191649-7191810 |
| | FORWARD LENGTH = 53 |
| 4342 | AT2G16900.1 Symbols: no symbol available: no full name available. Chr2: 7323857-7325374 |
| | REVERSE LENGTH = 382 |
| 4343 | AT2G17370.1 Symbols: HMGR2, HMG2: 3-hydroxy-3-methylglutaryl-CoA reductase 2,3-HYDROXY- |
| | 3-METHYLGLUTARYL-COENZYME A REDUCTASE 2. Chr2: 7550007-7551981 FORWARD |
| | LENGTH = 562 |
| 4344 | AT2G17440.1 Symbols: PIRL5: plant intracellular ras group-related LRR 5. Chr2: 7571331-7573406 |
| | FORWARD LENGTH = 526 |
| 4345 | AT2G17480.1 Symbols: MLO8, ATMLO8: MILDEW RESISTANCE LOCUS O 8. Chr2: 7590559- |
| | 7593768 REVERSE LENGTH = 593 |
| 4346 | AT2G17540.2 Symbols: no symbol available: no full name available. Chr2: 7630662-7631489 |
| | REVERSE LENGTH = 275 |
| 4347 | AT2G17550.1 Symbols: TRM26: TON1 Recruiting Motif 26. Chr2: 7634495-7637212 REVERSE |
| | LENGTH = 779 |
| 4348 | AT2G18150.1 Symbols: no symbol available: no full name available. Chr2: 7892298-7893586 |
| | REVERSE LENGTH = 338 |
| 4349 | AT2G18160.1 Symbols: GBF5, ATBZIP2, bZIP2, FTM3: FLORAL TRANSITION AT THE |
| | MERISTEM3, G-BOX BINDING FACTOR 5, basic leucine-zipper 2. Chr2: 7898288-7898803 REVERSE |
| | LENGTH = 171 |
| 4350 | AT2G18876.1 Symbols: no symbol available: no full name available. Chr2: 8169683-8172287 |
| | FORWARD LENGTH = 382 |
| 4351 | AT2G18970.1 Symbols: no symbol available: no full name available. Chr2: 8229373-8229670 |
| | FORWARD LENGTH = 62 |
| 4352 | AT2G19170.1 Symbols: SLP3: subtilisin-like serine protease 3. Chr2: 8314154-8317620 REVERSE |
| | LENGTH = 815 |
| 4353 | AT2G19390.1 Symbols: no symbol available: no full name available. Chr2: 8390136-8396477 |
| | REVERSE LENGTH = 1211 |
| 4354 | AT2G19470.1 Symbols: ckl5: casein kinase I-like 5. Chr2: 8433851-8436295 REVERSE LENGTH = 433 |
| 4355 | AT2G19490.1 Symbols: RECA2: A. thaliana recA homolog 2. Chr2: 8441732-8444006 FORWARD |
| | LENGTH = 430 |
| 4356 | AT2G19880.2 Symbols: GCS: GlcCer synthase. Chr2: 8581578-8585118 FORWARD LENGTH = 520 |
| 4357 | AT2G20010.2 Symbols: no symbol available: no full name available. Chr2: 8637977-8641184 |
| | REVERSE LENGTH = 952 |
| 4358 | AT2G20080.1 Symbols: TIE2: TCP Interactor containing EAR motif protein 2. Chr2: 8663115-8664735 |
| | REVERSE LENGTH = 178 |
| 4359 | AT2G20440.1 Symbols: no symbol available: no full name available. Chr2: 8811034-8813251 |
| | REVERSE LENGTH = 425 |
| 4360 | AT2G20680.1 Symbols: MAN2, AtMAN2, MAN5-2: endo-beta-mannase 2. Chr2: 8921024-8923066 |
| | FORWARD LENGTH = 433 |
| 4361 | AT2G20780.1 Symbols: no symbol available: no full name available. Chr2: 8947496-8949170 |
| | REVERSE LENGTH = 526 |
| 4362 | AT2G20815.1 Symbols: QWRF3: QWRF domain containing 3. Chr2: 8959988-8961917 FORWARD |
| | LENGTH = 482 |
| 4363 | AT2G20830.2 Symbols: no symbol available: no full name available. Chr2: 8968370-8970479 |
| | REVERSE LENGTH = 431 |
| 4364 | AT2G20990.3 Symbols: ATSYTA, SYTI, NTMC2TYPE1.1, NTMC2T1.1, SYTA: SYNAPTOTAGMIN |
| | 1, synaptotagmin A, ARABIDOPSIS THALIANA SYNAPTOTAGMIN A. Chr2: 9014827-9017829 |
| | FORWARD LENGTH = 579 |
| 4365 | AT2G21300.1 Symbols: no symbol available: no full name available. Chr2: 9114396-9118292 |
| | REVERSE LENGTH = 862 |
| 4366 | AT2G21520.2 Symbols: no symbol available: no full name available. Chr2: 9215956-9218953 |
| | FORWARD LENGTH = 637 |
| 4367 | AT2G21940.4 Symbols: SKI, ATSKI: shikimate kinase 1. Chr2: 9351106-9352881 FORWARD |
| | LENGTH = 304 |
| 4368 | AT2G22670.4 Symbols: IAA8: indoleacetic acid-induced protein 8. Chr2: 9636877-9638459 FORWARD |
| | LENGTH = 338 |
| 4369 | AT2G23680.1 Symbols: no symbol available: no full name available. Chr2: 10066242-10067164 |
| | FORWARD LENGTH = 189 |
| 4370 | AT2G23700.1 Symbols: no symbol available: no full name available. Chr2: 10076624-10079849 |
| | REVERSE LENGTH = 707 |
| 4371 | AT2G23985.2 Symbols: no symbol available: no full name available. Chr2: 10205658-10206283 |
| | FORWARD LENGTH = 71 |
| 4372 | AT2G24280.1 Symbols: no symbol available: no full name available. Chr2: 10334622-10336927 |
| | FORWARD LENGTH = 494 |
| 4373 | AT2G24390.1 Symbols: no symbol available: no full name available. Chr2: 10373796-10374654 |
| | FORWARD LENGTH = 152 |
| 4374 | AT2G24530.1 Symbols: no symbol available: no full name available. Chr2: 10422597-10423820 |
| | FORWARD LENGTH = 407 |
| 4375 | AT2G24630.1 Symbols: CSLC08, ATCSLC8, ATCSLC08: CELLULOSE-SYNTHASE LIKE C8. |
| | Chr2: 10471558-10473984 REVERSE LENGTH = 690 |
| 4376 | AT2G24640.1 Symbols: UBP19: ubiquitin-specific protease 19. Chr2: 10475613-10479341 REVERSE |
| | LENGTH-672 |
| 4377 | AT2G24945.1 Symbols: no symbol available: no full name available. Chr2: 10611582-10612214 |
| | FORWARD LENGTH = 210 |
| 4378 | AT2G25190.1 Symbols: no symbol available: no full name available. Chr2: 10734187-10735426 |
| | FORWARD LENGTH = 240 |
| 4379 | AT2G25510.1 Symbols: no symbol available: no full name available. Chr2: 10856772-10857083 |
| | FORWARD LENGTH = 103 |
| 4380 | AT2G25700.1 Symbols: SK3, ASK3: SKP1-like 3. Chr2: 10948914-10949551 REVERSE LENGTH = 163 |
| 4381 | AT2G26430.1 Symbols: RCY1, ATRCY1, MOS12: arginine-rich cyclin 1, modifier of snc1 |
| | 12, ARGININE-RICH CYCLIN 1. Chr2: 11243433-11245504 REVERSE LENGTH = 416 |
| 4382 | AT2G27060.1 Symbols: no symbol available: no full name available. Chr2: 11551288-11554577 |
| | FORWARD LENGTH = 1020 |
| 4383 | AT2G27110.1 Symbols: FRS3: FAR1-related sequence 3. Chr2: 11576969-11580405 REVERSE |
| | LENGTH = 851 |
| 4384 | AT2G27230.1 Symbols: LHW: LONESOME HIGHWAY. Chr2: 11650895-11653840 FORWARD |
| | LENGTH = 650 |
| 4385 | AT2G27340.2 Symbols: no symbol available: no full name available. Chr2: 11697449-11699120 |
| | FORWARD LENGTH = 258 |
| 4386 | AT2G27350.5 Symbols: OTLD1: otubain-like deubiquitinase 1. Chr2: 11699780-11702363 REVERSE |
| | LENGTH = 506 |
| 4387 | AT2G27402.1 Symbols: no symbol available: no full name available. Chr2: 11724031-11724189 |
| | FORWARD LENGTH = 52 |
| 4388 | AT2G27830.1 Symbols: no symbol available: no full name available. Chr2: 11860734-11861306 |
| | FORWARD LENGTH = 190 |
| 4389 | AT2G27950.1 Symbols: no symbol available: no full name available. Chr2: 11899422-11903171 |
| | REVERSE LENGTH = 839 |
| 4390 | AT2G28105.1 Symbols: no symbol available: no full name available. Chr2: 11976764-11977713 |
| | FORWARD LENGTH = 166 |
| 4391 | AT2G28320.1 Symbols: no symbol available: no full name available. Chr2: 12095161-12099424 |
| | FORWARD LENGTH = 737 |
| 4392 | AT2G28380.1 Symbols: DRB2, AtDRB2: dsRNA-binding protein 2. Chr2: 12134098-12135915 |
| | REVERSE LENGTH = 434 |
| 4393 | AT2G28490.1 Symbols: no symbol available: no full name available. Chr2: 12178812-12180983 |
| | REVERSE LENGTH = 511 |
| 4394 | AT2G28550.3 Symbols: TOE1, RAP2.7: TARGET OF EARLY ACTIVATION TAGGED (EAT) |
| | 1, related to AP2.7. Chr2: 12226168-12228251 REVERSE LENGTH = 464 |
| 4395 | AT2G28810.1 Symbols: no symbol available: no full name available. Chr2: 12363681-12365080 |
| | FORWARD LENGTH = 340 |
| 4396 | AT2G29190.1 Symbols: PUM2, APUM2: pumilio 2. Chr2: 12544260-12548071 REVERSE |
| | LENGTH = 972 |
| 4397 | AT2G29290.2 Symbols: no symbol available: no full name available. Chr2: 12586498-12587684 |
| | FORWARD LENGTH = 262 |
| 4398 | AT2G29600.1 Symbols: no symbol available: no full name available. Chr2: 12655661-12656908 |
| | FORWARD LENGTH = 415 |
| 4399 | AT2G30370.1 Symbols: AtEPFL6, EPFL6, CHAL: EPF1-like 6, CHALLAH. Chr2: 12940577-12942167 |
| | REVERSE LENGTH = 230 |
| 4400 | AT2G30520.1 Symbols: RPT2: ROOT PHOTOTROPISM 2. Chr2: 13002920-13005573 REVERSE |
| | LENGTH = 593 |
| 4401 | AT2G30590.1 Symbols: WRKY21: WRKY DNA-binding protein 21. Chr2: 13033891-13035303 |
| | FORWARD LENGTH = 380 |
| 4402 | AT2G30600.5 Symbols: no symbol available: no full name available. Chr2: 13037410-13041475 |
| | FORWARD LENGTH = 855 |
| 4403 | AT2G30942.1 Symbols: ssSPTb: small subunit of SPT b. Chr2: 13170953-13171835 FORWARD |
| | LENGTH = 56 |
| 4404 | AT2G31010.1 Symbols: no symbol available: no full name available. Chr2: 13194939-13199642 |
| | FORWARD LENGTH = 775 |
| 4405 | AT2G31070.1 Symbols: TCP10: TCP domain protein 10. Chr2: 13220984-13222069 REVERSE |
| | LENGTH = 361 |
| 4406 | AT2G31280.3 Symbols: LL2, CPUORF7, LHL2: conserved peptide upstream open reading frame |
| | 7, LONESOME HIGHWAY LIKE 2. Chr2: 13339678-13343424 FORWARD LENGTH = 737 |
| 4407 | AT2G31490.1 Symbols: no symbol available: no full name available. Chr2: 13412060-13413002 |
| | FORWARD LENGTH = 71 |
| 4408 | AT2G32190.1 Symbols: no symbol available: no full name available. Chr2: 13674588-13675092 |
| | FORWARD LENGTH = 71 |
| 4409 | AT2G32235.1 Symbols: no symbol available: no full name available. Chr2: 13682823-13684199 |
| | REVERSE LENGTH = 310 |
| 4410 | AT2G32250.1 Symbols: FRS2: FAR1-related sequence 2. Chr2: 13693518-13696783 FORWARD |
| | LENGTH = 807 |
| 4411 | AT2G32320.2 Symbols: no symbol available: no full name available. Chr2: 13727938-13731428 |
| | FORWARD LENGTH = 537 |
| 4412 | AT2G32430.1 Symbols: no symbol available: no full name available. Chr2: 13771626-13774102 |
| | FORWARD LENGTH = 409 |
| 4413 | AT2G32440.1 Symbols: CYP88A4, KAO2, ATKAO2: ARABIDOPSIS ENT-KAURENOIC ACID |
| | HYDROXYLASE 2, ent-kaurenoic acid hydroxylase 2. Chr2: 13775668-13777783 FORWARD |
| | LENGTH = 489 |
| 4414 | AT2G32890.1 Symbols: RALFL17: RALF-like 17. Chr2: 13953058-13953291 REVERSE LENGTH = 77 |
| 4415 | AT2G33170.1 Symbols: no symbol available: no full name available. Chr2: 14056371-14059829 |
| | REVERSE LENGTH = 1124 |
| 4416 | AT2G33390.1 Symbols: no symbol available: no full name available. Chr2: 14151714-14152698 |
| | FORWARD LENGTH = 98 |
| 4417 | AT2G33700.1 Symbols: PP2CG1: protein phosphatase 2C G Group 1. Chr2: 14254200-14255784 |
| | FORWARD LENGTH = 380 |
| 4418 | AT2G33770.1 Symbols: PHO2, ATUBC24, UBC24: UBIQUITIN-CONJUGATING ENZYME |
| | 24, phosphate 2. Chr2: 14277785-14281482 REVERSE LENGTH = 907 |
| 4419 | AT2G34260.1 Symbols: WDR55: human WDR55 (WD40 repeat) homolog. Chr2: 14465899-14468416 |
| | FORWARD LENGTH = 353 |
| 4420 | AT2G34660.1 Symbols: EST4, ATMRP2, MRP2, AtABCC2, ABCC2: ATP-binding cassette C2, multidrug |
| | resistance-associated protein 2, Arabidopsis thaliana ATP-binding cassette C2. Chr2: 14603267-14612387 |
| | FORWARD LENGTH = 1623 |
| 4421 | AT2G34670.2 Symbols: no symbol available: no full name available. Chr2: 14612741-14615231 |
| | REVERSE LENGTH = 694 |
| 4422 | AT2G34780.1 Symbols: EMB1611, MEE22: MATERNAL EFFECT EMBRYO ARREST 22, EMBRYO |
| | DEFECTIVE 1611. Chr2: 14668653-14673607 FORWARD LENGTH = 1297 |
| 4423 | AT2G35155.1 Symbols: no symbol available: no full name available. Chr2: 14819209-14821330 |
| | REVERSE LENGTH = 579 |
| 4424 | AT2G35310.1 Symbols: REM23: reproductive meristem 23. Chr2: 14864943-14866404 FORWARD |
| | LENGTH = 288 |
| 4425 | AT2G35390.2 Symbols: no symbol available: no full name available. Chr2: 14895528-14897581 |
| | REVERSE LENGTH = 403 |
| 4426 | AT2G35510.1 Symbols: SRO1: similar to RCD one 1. Chr2: 14916898-14919198 REVERSE |
| | LENGTH = 568 |
| 4427 | AT2G35600.1 Symbols: BRXL1, ATBRXL1: ARABIDOPSIS THALIANA BREVIS RADIX LIKE |
| | 1, BREVIS RADIX-like 1. Chr2: 14941092-14943281 REVERSE LENGTH = 331 |
| 4428 | AT2G35660.1 Symbols: CTF2A.: Chr2: 14988499-14990320 FORWARD LENGTH = 439 |
| 4429 | AT2G35680.1 Symbols: no symbol available: no full name available. Chr2: 14997004-14998590 |
| | REVERSE LENGTH = 337 |
| 4430 | AT2G35750.1 Symbols: no symbol available: no full name available. Chr2: 15029904-15030089 |
| | REVERSE LENGTH = 61 |
| 4431 | AT2G35800.1 Symbols: SAMTL: S-adenosyl methionine transporter-like. Chr2: 15044437-15048352 |
| | FORWARD LENGTH = 823 |
| 4432 | AT2G35880.1 Symbols: no symbol available: no full name available. Chr2: 15063204-15065259 |
| | REVERSE LENGTH = 432 |
| 4433 | AT2G35940.1 Symbols: EDA29, BLH1: BEL1-like homeodomain 1, embryo sac development arrest 29. |
| | Chr2: 15089171-15091699 REVERSE LENGTH = 680 |
| 4434 | AT2G36210.1 Symbols: SAUR45: SMALL AUXIN UPREGULATED RNA 45. Chr2: 15186326- |
| | 15186733 REVERSE LENGTH = 135 |
| 4435 | AT2G36370.1 Symbols: no symbol available: no full name available. Chr2: 15247768-15252800 |
| | FORWARD LENGTH = 940 |
| 4436 | AT2G36460.1 Symbols: FBA6: fructose-bisphosphate aldolase 6. Chr2: 15296929-15298387 REVERSE |
| | LENGTH = 358 |
| 4437 | AT2G36470.1 Symbols: no symbol available: no full name available. Chr2: 15299385-15300368 |
| | REVERSE LENGTH = 327 |
| 4438 | AT2G36720.1 Symbols: no symbol available: no full name available. Chr2: 15393447-15399189 |
| | FORWARD LENGTH = 1007 |
| 4439 | AT2G36840.1 Symbols: ACR10: ACT domain repeats 10. Chr2: 15451834-15453550 REVERSE |
| | LENGTH = 410 |
| 4440 | AT2G36960.3 Symbols: TKI1: TSL-kinase interacting protein 1. Chr2: 15523552-15527245 FORWARD |
| | LENGTH = 744 |
| 4441 | AT2G37080.1 Symbols: RIP2: ROP interactive partner 2. Chr2: 15581565-15584057 REVERSE |
| | LENGTH = 583 |
| 4442 | AT2G37150.3 Symbols: no symbol available: no full name available. Chr2: 15603748-15607600 |
| | REVERSE LENGTH = 599 |
| 4443 | AT2G37160.2 Symbols: no symbol available: no full name available. Chr2: 15609047-15612529 |
| | FORWARD LENGTH = 573 |
| 4444 | AT2G37340.1 Symbols: RS2Z33, RSZ33, ATRSZ33, AT-RS2Z33: arginine/serine-rich zinc knuckle- |
| | containing protein 33, ARGININE/SERINE-RICH ZINC KNUCKLE-CONTAINING PROTEIN 33. |
| | Chr2: 15670372-15672331 REVERSE LENGTH = 290 |
| 4445 | AT2G37480.1 Symbols: no symbol available: no full name available. Chr2: 15738645-15739579 |
| | FORWARD LENGTH = 194 |
| 4446 | AT2G37570.1 Symbols: SLT1: sodium- and lithium-tolerant 1. Chr2: 15761466-15762950 REVERSE |
| | LENGTH = 494 |
| 4447 | AT2G37840.1 Symbols: no symbol available: no full name available. Chr2: 15851978-15856047 |
| | FORWARD LENGTH = 733 |
| 4448 | AT2G37890.1 Symbols: no symbol available: no full name available. Chr2: 15862017-15863849 |
| | REVERSE LENGTH = 337 |
| 4449 | AT2G38040.1 Symbols: CAC3: acetyl Co-enzyme a carboxylase carboxyltransferase alpha subunit. |
| | Chr2: 15917612-15920749 FORWARD LENGTH = 769 |
| 4450 | AT2G38290.1 Symbols: ATAMT2, AMT2, AMT2 1: ammonium transporter 2, AMMONIUM |
| | TRANSPORTER 2 1. Chr2: 16039672-16042291 REVERSE LENGTH = 475 |
| 4451 | AT2G38820.2 Symbols: no symbol available: no full name available. Chr2: 16222271-16223393 |
| | FORWARD LENGTH = 310 |
| 4452 | AT2G39280.2 Symbols: no symbol available: no full name available. Chr2: 16402001-16406650 |
| | REVERSE LENGTH = 772 |
| 4453 | AT2G39970.1 Symbols: PXN, APEM3, PMP38: peroxisomal NAD carrier, peroxisomal membrane protein |
| | 38, ABERRANT PEROXISOME MORPHOLOGY 3. Chr2: 16684026-16686392 REVERSE |
| | LENGTH = 331 |
| 4454 | AT2G40120.1 Symbols: no symbol available: no full name available. Chr2: 16755137-16757258 |
| | REVERSE LENGTH = 570 |
| 4455 | AT2G40460.1 Symbols: no symbol available: no full name available. Chr2: 16897123-16901171 |
| | FORWARD LENGTH = 583 |
| 4456 | AT2G41100.1 Symbols: TCH3, CML12, ATCAL4: ARABIDOPSIS THALIANA CALMODULIN LIKE |
| | 4, calmodulin-like 12, TOUCH 3. Chr2: 17138131-17139406 FORWARD LENGTH = 324 |
| 4457 | AT2G41430.1 Symbols: ERD15, CID1, LSR1: CTC-Interacting Domain 1, LIGHT STRESS- |
| | REGULATED I, EARLY RESPONSIVE TO DEHYDRATION 15. Chr2: 17269736-172703 14 |
| | FORWARD LENGTH = 163 |
| 4458 | AT2G41440.1 Symbols: no symbol available: no full name available. Chr2: 17271792-17274065 |
| | FORWARD LENGTH = 289 |
| 4459 | AT2G41710.3 Symbols: no symbol available: no full name available. Chr2: 17400274-17402903 |
| | REVERSE LENGTH = 458 |
| 4460 | AT2G41830.1 Symbols: no symbol available: no full name available. Chr2: 17450431-17456453 |
| | REVERSE LENGTH = 1025 |
| 4461 | AT2G42030.1 Symbols: no symbol available: no full name available. Chr2: 17539069-17540346 |
| | REVERSE LENGTH = 425 |
| 4462 | AT2G42300.1 Symbols: no symbol available: no full name available. Chr2: 17621542-17624635 |
| | FORWARD LENGTH = 327 |
| 4463 | AT2G42320.1 Symbols: no symbol available: no full name available. Chr2: 17628102-17630657 |
| | FORWARD LENGTH = 669 |
| 4464 | AT2G42380.2 Symbols: BZIP34, ATBZIP34.: Chr2: 17647205-17648660 REVERSE LENGTH = 321 |
| 4465 | AT2G42880.1 Symbols: MPK20, ATMPK20: MAP kinase 20. Chr2: 17840572-17843947 REVERSE |
| | LENGTH = 606 |
| 4466 | AT2G43320.1 Symbols: no symbol available: no full name available. Chr2: 17996874-17999158 |
| | REVERSE LENGTH = 351 |
| 4467 | AT2G43430.1 Symbols: GLX2-1, GLY1: glyoxalase 2-1, GLYOXALASE II. Chr2: 18035569-18038064 |
| | REVERSE LENGTH = 331 |
| 4468 | AT2G43745.1 Symbols: no symbol available: no full name available. Chr2: 18128859-18129355 |
| | REVERSE LENGTH = 136 |
| 4469 | AT2G44090.1 Symbols: no symbol available: no full name available. Chr2: 18238682-18240828 |
| | REVERSE LENGTH = 582 |
| 4470 | AT2G44410.1 Symbols: no symbol available: no full name available. Chr2: 18328873-18330114 |
| | FORWARD LENGTH = 413 |
| 4471 | AT2G44650.1 Symbols: CHL-CPN10, CPN10: CHLOROPLAST CHAPERONIN 10, chloroplast |
| | chaperonin 10. Chr2: 18419521-18420510 REVERSE LENGTH = 139 |
| 4472 | AT2G44670.1 Symbols: no symbol available: no full name available. Chr2: 18425279-18425673 |
| | FORWARD LENGTH = 93 |
| 4473 | AT2G44850.1 Symbols: no symbol available: no full name available. Chr2: 18498672-18500823 |
| | FORWARD LENGTH = 400 |
| 4474 | AT2G44900.1 Symbols: ARABIDILLO-1, ARABIDILLO1: F-box Armadillo protein 1, ARABIDILLO-1. |
| | Chr2: 18511719-18515762 REVERSE LENGTH = 930 |
| 4475 | AT2G45010.1 Symbols: no symbol available: no full name available. Chr2: 18568045-18569615 |
| | FORWARD LENGTH = 244 |
| 4476 | AT2G45380.1 Symbols: no symbol available: no full name available. Chr2: 18699588-18703093 |
| | REVERSE LENGTH = 494 |
| 4477 | AT2G45460.3 Symbols: no symbol available: no full name available. Chr2: 18737054-18741690 |
| | REVERSE LENGTH = 916 |
| 4478 | AT2G45600.1 Symbols: no symbol available: no full name available. Chr2: 18789799-18790788 |
| | FORWARD LENGTH = 329 |
| 4479 | AT2G45850.1 Symbols: AHL9: AT-hook motif nuclear localized protein 9. Chr2: 18871901-18873457 |
| | REVERSE LENGTH = 348 |
| 4480 | AT2G46210.1 Symbols: AtSLD2, SLD2: sphingoid LCB desaturase 2. Chr2: 18977542-18978891 |
| | FORWARD LENGTH = 449 |
| 4481 | AT2G46500.1 Symbols: ATPI4K GAMMA 4, UBDK GAMMA 4, PI4K GAMMA 4: phosphoinositide 4- |
| | kinase gamma 4, UBIQUITIN-LIKE DOMAIN KINASE GAMMA 4. Chr2: 19086741-19088534 |
| | REVERSE LENGTH = 566 |
| 4482 | AT2G46590.2 Symbols: DAG2: DOF AFFECTING GERMINATION 2. Chr2: 19133166-19134905 |
| | FORWARD LENGTH = 369 |
| 4483 | AT2G46830.1 Symbols: CCA1, AtCCA1: circadian clock associated 1. Chr2: 19246005-19248717 |
| | FORWARD LENGTH = 608 |
| 4484 | AT2G47020.1 Symbols: no symbol available: no full name available. Chr2: 19320090-19322279 |
| | REVERSE LENGTH = 413 |
| 4485 | AT2G47160.2 Symbols: AtBORI, BORI: REQUIRES HIGH BORON 1. Chr2: 19357740-19360787 |
| | REVERSE LENGTH = 729 |
| 4486 | AT2G47410.1 Symbols: no symbol available: no full name available. Chr2: 19449133-19456991 |
| | FORWARD LENGTH = 1520 |
| 4487 | AT2G47490.1 Symbols: ATNDT1, NDT1: ARABIDOPSIS THALIANA NAD+ TRANSPORTER |
| | 1, NAD+ transporter 1. Chr2: 19487549-19489311 FORWARD LENGTH = 312 |
| 4488 | AT2G47850.1 Symbols: no symbol available: no full name available. Chr2: 19595953-19598119 |
| | FORWARD LENGTH = 468 |
| 4489 | AT2G47900.3 Symbols: AtTLP3, TLP3: tubby like protein 3. Chr2: 19611196-19612766 REVERSE |
| | LENGTH = 407 |
| 4490 | AT2G47930.1 Symbols: AGP26, ATAGP26: ARABIDOPSIS THALIANA ARABINOGALACTAN |
| | PROTEIN 26, arabinogalactan protein 26. Chr2: 19617219-19617629 REVERSE LENGTH = 136 |
| 4491 | AT3G01090.2 Symbols: KIN10, SNRK1.1, AKIN10: SNF1 kinase homolog 10, SNF1-RELATED |
| | PROTEIN KINASE 1.1. Chr3: 31437-34143 REVERSE LENGTH = 535 |
| 4492 | AT3G01130.1 Symbols: no symbol available: no full name available. Chr3: 44714-45522 REVERSE |
| | LENGTH = 53 |
| 4493 | AT3G01170.1 Symbols: no symbol available: no full name available. Chr3: 58043-58690 FORWARD |
| | LENGTH = 215 |
| 4494 | AT3G01470.1 Symbols: HD-ZIP-1, ATHB-1, ATHB1, HAT5, HB-1: homeobox 1, HOMEODOMAIN |
| | PROTEIN FROM ARABIDOIPSIS THALIANA 5, ARABIDOPSIS THALIANA HOMEOBOX 1. |
| | Chr3: 182648-184034 REVERSE LENGTH = 272 |
| 4495 | AT3G01810.1 Symbols: no symbol available: no full name available. Chr3: 289218-292557 FORWARD |
| | LENGTH = 921 |
| 4496 | AT3G01980.3 Symbols: no symbol available: no full name available. Chr3: 327747-328833 REVERSE |
| | LENGTH = 296 |
| 4497 | AT3G02065.2 Symbols: no symbol available: no full name available. Chr3: 359136-360734 FORWARD |
| | LENGTH = 505 |
| 4498 | AT3G02470.1 Symbols: SAMDC: S-adenosylmethionine decarboxylase. Chr3: 510223-511323 |
| | FORWARD LENGTH = 366 |
| 4499 | AT3G02750.3 Symbols: no symbol available: no full name available. Chr3: 593601-595457 REVERSE |
| | LENGTH = 527 |
| 4500 | AT3G03300.1 Symbols: ATDCL2, DCL2: DICER-LIKE 2, dicer-like 2. Chr3: 768020-774833 REVERSE |
| | LENGTH = 1388 |
| 4501 | AT3G03890.1 Symbols: no symbol available: no full name available. Chr3: 999667-1001996 REVERSE |
| | LENGTH = 321 |
| 4502 | AT3G03970.1 Symbols: SINE2.: Chr3: 1028144-1029891 REVERSE LENGTH = 554 |
| 4503 | AT3G04450.1 Symbols: no symbol available: no full name available. Chr3: 1184302-1186264 |
| | FORWARD LENGTH = 442 |
| 4504 | AT3G04580.1 Symbols: EIN4: ETHYLENE INSENSITIVE 4. Chr3: 1235576-1237965 REVERSE |
| | LENGTH = 766 |
| 4505 | AT3G04670.1 Symbols: WRKY39, ATWRKY39: WRKY DNA-BINDING PROTEIN 39, WRKY DNA- |
| | binding protein 39. Chr3: 1266530-1267691 REVERSE LENGTH = 330 |
| 4506 | AT3G04810.1 Symbols: ATNEK2, NEK2: NIMA-related kinase 2. Chr3: 1318096-1321101 FORWARD |
| | LENGTH = 606 |
| 4507 | AT3G04910.1 Symbols: ZIK4, WNK1, ATWNK1: with no lysine (K) kinase 1. Chr3: 1355084-1358057 |
| | FORWARD LENGTH = 700 |
| 4508 | AT3G05030.1 Symbols: NHX2, ATNHX2: sodium hydrogen exchanger 2. Chr3: 1393456-1396784 |
| | REVERSE LENGTH = 546 |
| 4509 | AT3G05160.1 Symbols: no symbol available: no full name available. Chr3: 1453267-1456997 |
| | REVERSE LENGTH = 458 |
| 4510 | AT3G05380.2 Symbols: ALY2, ATALY2: ARABIDOPSIS THALIANA ALWAYS EARLY |
| | 2, ALWAYS EARLY 2. Chr3: 1540562-1546139 FORWARD LENGTH = 1052 |
| 4511 | AT3G05560.1 Symbols: no symbol available: no full name available. Chr3: 1614641-1615204 |
| | FORWARD LENGTH = 124 |
| 4512 | AT3G05580.1 Symbols: TOPP9: type one protein phosphatase 9. Chr3: 1618216-1619850 REVERSE |
| | LENGTH = 318 |
| 4513 | AT3G05690.1 Symbols: ATHAP2B, AtNF-YA2, UNE8, HAP2B, NF-YA2: UNFERTILIZED EMBRYO |
| | SAC 8, HEME ACTIVATOR PROTEIN (YEAST) HOMOLOG 2B, “nuclear factor Y, subunit A2”. |
| | Chr3: 1676922-1678324 REVERSE LENGTH = 295 |
| 4514 | AT3G05710.2 Symbols: ATSYP43, SYP43: syntaxin of plants 43. Chr3: 1685262-1687229 FORWARD |
| | LENGTH = 331 |
| 4515 | AT3G05750.1 Symbols: TRM6: TON1 Recruiting Motif 6. Chr3: 1704677-1707546 FORWARD |
| | LENGTH = 801 |
| 4516 | AT3G05937.1 Symbols: no symbol available: no full name available. Chr3: 1776158-1776319 |
| | REVERSE LENGTH = 53 |
| 4517 | AT3G06270.1 Symbols: no symbol available: no full name available. Chr3: 1896763-1897887 |
| | FORWARD LENGTH = 348 |
| 4518 | AT3G06360.1 Symbols: AGP27, ATAGP27: arabinogalactan protein 27, ARABINOGALACTAN |
| | PROTEIN 27. Chr3: 1928976-1929353 FORWARD LENGTH = 125 |
| 4519 | AT3G06620.1 Symbols: no symbol available: no full name available. Chr3: 2062833-2067138 |
| | REVERSE LENGTH = 773 |
| 4520 | AT3G07100.1 Symbols: SEC24A, ERMO2, AtSEC24A: ENDOPLASMIC RETICULUM |
| | MORPHOLOGY 2. Chr3: 2245689-2250077 REVERSE LENGTH = 1038 |
| 4521 | AT3G07170.1 Symbols: no symbol available: no full name available. Chr3: 2280428-2282097 |
| | FORWARD LENGTH = 203 |
| 4522 | AT3G07210.1 Symbols: no symbol available: no full name available. Chr3: 2293000-2295119 |
| | REVERSE LENGTH = 547 |
| 4523 | AT3G07550.1 Symbols: no symbol available: no full name available. Chr3: 2409946-2411133 |
| | FORWARD LENGTH = 395 |
| 4524 | AT3G08510.1 Symbols: ATPLC2, PLC2: phospholipase C 2. Chr3: 2582626-2585556 REVERSE |
| | LENGTH = 581 |
| 4525 | AT3G08680.1 Symbols: no symbol available: no full name available. Chr3: 2638591-2640590 |
| | FORWARD LENGTH = 640 |
| 4526 | AT3G08730.1 Symbols: PK1, PK6, S6K1, ATPK1, ATPK6, ATS6K1: ARABIDOPSIS THALIANA |
| | PROTEIN-SERINE KINASE 1, ROTEIN-SERINE KINASE 6, P70 RIBOSOMAL S6 |
| | KINASE, ARABIDOPSIS THALIANA PROTEIN-SERINE KINASE 6, protein-serine kinase 1. |
| | Chr3: 2651581-2653363 REVERSE LENGTH = 465 |
| 4527 | AT3G08980.1 Symbols: no symbol available: no full name available. Chr3: 2741279-2742375 |
| | FORWARD LENGTH = 154 |
| 4528 | AT3G09150.2 Symbols: HY2, ATHY2, GUN3: GENOMES UNCOUPLED 3, ARABIDOPSIS |
| | ELONGATED HYPOCOTYL 2, ELONGATED HYPOCOTYL 2. Chr3: 2803665-2805333 FORWARD |
| | LENGTH = 329 |
| 4529 | AT3G09350.1 Symbols: Fes1A: Fes1A. Chr3: 2871216-2873109 FORWARD LENGTH = 363 |
| 4530 | AT3G09560.2 Symbols: ATPAH1, PAH1: PHOSPHATIDIC ACID PHOSPHOHYDROLASE 1. |
| | Chr3: 2934953-2938673 REVERSE LENGTH = 904 |
| 4531 | AT3G09820.1 Symbols: ATADK1, ADK1: adenosine kinase 1. Chr3: 3012122-3014624 FORWARD |
| | LENGTH = 344 |
| 4532 | AT3G09920.1 Symbols: PIP5K9: phosphatidyl inositol monophosphate 5 kinase. Chr3: 3040426- |
| | 3043676 REVERSE LENGTH = 815 |
| 4533 | AT3G10640.1 Symbols: VPS60.1.: Chr3: 3323501-3324905 REVERSE LENGTH = 235 |
| 4534 | AT3G10910.1 Symbols: DAFL1: DAF-Like gene 1. Chr3: 3413068-3413613 REVERSE LENGTH = 181 |
| 4535 | AT3G10915.5 Symbols: no symbol available: no full name available. Chr3: 3416101-3417497 |
| | REVERSE LENGTH = 249 |
| 4536 | AT3G10930.1 Symbols: no symbol available: no full name available. Chr3: 3420228-3420533 |
| | REVERSE LENGTH = 101 |
| 4537 | AT3G11200.1 Symbols: AL2: alfin-like 2. Chr3: 3508387-3510418 REVERSE LENGTH = 246 |
| 4538 | AT3G11320.1 Symbols: no symbol available: no full name available. Chr3: 3547017-3548539 |
| | REVERSE LENGTH = 308 |
| 4539 | AT3G11530.1 Symbols: no symbol available: no full name available. Chr3: 3628801-3629885 |
| | REVERSE LENGTH = 113 |
| 4540 | AT3G11850.1 Symbols: no symbol available: no full name available. Chr3: 3739222-3741101 |
| | REVERSE LENGTH = 504 |
| 4541 | AT3G12200.2 Symbols: Nek7, AtNek7: NIMA-related kinase 7. Chr3: 3887173-3890550 REVERSE |
| | LENGTH = 581 |
| 4542 | AT3G12280.1 Symbols: ATRBR1, RBR1, RB1, RB, RBR: RETINOBLASTOMA- |
| | RELATED, RETINOBLASTOMA 1, RETINOBLASTOMA-RELATED PROTEIN 1, retinoblastoma- |
| | related 1. Chr3: 3913671-3918433 REVERSE LENGTH = 1013 |
| 4543 | AT3G12550.1 Symbols: FDM3: factor of DNA methylation 3. Chr3: 3978669-3981372 FORWARD |
| | LENGTH = 638 |
| 4544 | AT3G12570.1 Symbols: FYD.: Chr3: 3989407-3990876 FORWARD LENGTH = 489 |
| 4545 | AT3G12620.1 Symbols: no symbol available: no full name available. Chr3: 4009510-4010993 |
| | REVERSE LENGTH = 385 |
| 4546 | AT3G12770.1 Symbols: MEF22: mitochondrial editing factor 22. Chr3: 4057027-4059193 REVERSE |
| | LENGTH = 694 |
| 4547 | AT3G13000.2 Symbols: no symbol available: no full name available. Chr3: 4158214-4160989 |
| | REVERSE LENGTH = 582 |
| 4548 | AT3G13030.1 Symbols: no symbol available: no full name available. Chr3: 4169675-4171417 |
| | REVERSE LENGTH = 544 |
| 4549 | AT3G13190.1 Symbols: no symbol available: no full name available. Chr3: 4240751-4241804 |
| | FORWARD LENGTH = 316 |
| 4550 | AT3G13430.1 Symbols: no symbol available: no full name available. Chr3: 4367754-4368701 |
| | FORWARD LENGTH = 315 |
| 4551 | AT3G13440.1 Symbols: no symbol available: no full name available. Chr3: 4378338-4379347 |
| | FORWARD LENGTH = 278 |
| 4552 | AT3G13580.3 Symbols: no symbol available: no full name available. Chr3: 4433809-4435109 |
| | FORWARD LENGTH = 244 |
| 4553 | AT3G13690.1 Symbols: no symbol available: no full name available. Chr3: 4486920-4490011 |
| | FORWARD LENGTH = 753 |
| 4554 | AT3G13700.1 Symbols: no symbol available: no full name available. Chr3: 4491008-4492632 |
| | REVERSE LENGTH = 296 |
| 4555 | AT3G13857.1 Symbols: no symbol available: no full name available. Chr3: 4561171-4561299 |
| | FORWARD LENGTH = 42 |
| 4556 | AT3G14010.1 Symbols: CID4: CTC-interacting domain 4. Chr3: 4637164-4640691 FORWARD |
| | LENGTH = 595 |
| 4557 | AT3G14020.1 Symbols: NF-YA6: “nuclear factor Y, subunit A6”. Chr3: 4642968-4644301 FORWARD |
| | LENGTH = 308 |
| 4558 | AT3G14110.3 Symbols: FLU: FLUORESCENT IN BLUE LIGHT. Chr3: 4676222-4677602 REVERSE |
| | LENGTH = 317 |
| 4559 | AT3G14172.1 Symbols: no symbol available: no full name available. Chr3: 4698860-4704773 |
| | REVERSE LENGTH = 1270 |
| 4560 | AT3G14415.2 Symbols: GOX2: glycolate oxidase 2. Chr3: 4818667-4820748 FORWARD |
| | LENGTH = 373 |
| 4561 | AT3G14590.2 Symbols: NTMC2TYPE6.2, NTMC2T6.2.: Chr3: 4904448-4907741 REVERSE |
| | LENGTH = 737 |
| 4562 | AT3G15030.1 Symbols: MEE35, TCP4: TCP family transcription factor 4, maternal effect embryo arrest |
| | 35. Chr3: 5062308-5063570 FORWARD LENGTH = 420 |
| 4563 | AT3G15220.1 Symbols: no symbol available: no full name available. Chr3: 5126899-5131752 |
| | REVERSE LENGTH = 690 |
| 4564 | AT3G15350.1 Symbols: no symbol available: no full name available. Chr3: 5167250-5168882 |
| | FORWARD LENGTH = 424 |
| 4565 | AT3G15351.1 Symbols: no symbol available: no full name available. Chr3: 5176726-5177531 |
| | FORWARD LENGTH = 165 |
| 4566 | AT3G15354.1 Symbols: SPA3: SPA1-related 3. Chr3: 5169327-5172480 REVERSE LENGTH = 837 |
| 4567 | AT3G15430.1 Symbols: no symbol available: no full name available. Chr3: 5209408-5211562 |
| | FORWARD LENGTH = 488 |
| 4568 | AT3G17120.1 Symbols: no symbol available: no full name available. Chr3: 5842410-5843246 |
| | FORWARD LENGTH = 219 |
| 4569 | AT3G17310.2 Symbols: DRM3, AtDRM3: domains rearranged methyltransferase 3. Chr3: 5909294- |
| | 5912838 REVERSE LENGTH = 710 |
| 4570 | AT3G17450.1 Symbols: no symbol available: no full name available. Chr3: 5972793-5975684 |
| | REVERSE LENGTH = 877 |
| 4571 | AT3G17580.1 Symbols: no symbol available: no full name available. Chr3: 6015276-6015518 |
| | FORWARD LENGTH = 80 |
| 4572 | AT3G17609.2 Symbols: HYH: HY5-homolog. Chr3: 6023971-6024585 FORWARD LENGTH = 149 |
| 4573 | AT3G17611.1 Symbols: ATRBL10, RBL14, ATRBL14, RBL10: RHOMBOID-like protein |
| | 14, RHOMBOID-like protein 10. Chr3: 6024946-6026173 FORWARD LENGTH = 334 |
| 4574 | AT3G17950.1 Symbols: no symbol available: no full name available. Chr3: 6146311-6147169 |
| | FORWARD LENGTH = 211 |
| 4575 | AT3G18160.3 Symbols: PEX3-1: peroxin 3-1. Chr3: 6220918-6222923 FORWARD LENGTH = 376 |
| 4576 | AT3G18370.1 Symbols: NTMC2T3, NTMC2TYPE3, SYTF, ATSYTF.: Chr3: 6306362-6310256 |
| | FORWARD LENGTH = 815 |
| 4577 | AT3G18620.1 Symbols: no symbol available: no full name available. Chr3: 6408849-6410716 |
| | FORWARD LENGTH = 345 |
| 4578 | AT3G18770.1 Symbols: no symbol available: no full name available. Chr3: 6460026-6462510 |
| | REVERSE LENGTH = 625 |
| 4579 | AT3G19250.1 Symbols: no symbol available: no full name available. Chr3: 6666842-6668008 |
| | REVERSE LENGTH = 360 |
| 4580 | AT3G19440.1 Symbols: no symbol available: no full name available. Chr3: 6740778-6743132 |
| | FORWARD LENGTH = 477 |
| 4581 | AT3G20040.1 Symbols: ATHXK4, HKL2: HEXOKINASE-LIKE 2. Chr3: 6995317-6998064 |
| | FORWARD LENGTH = 502 |
| 4582 | AT3G20200.1 Symbols: no symbol available: no full name available. Chr3: 7047895-7051145 |
| | FORWARD LENGTH = 780 |
| 4583 | AT3G20260.1 Symbols: no symbol available: no full name available. Chr3: 7064190-7065751 |
| | REVERSE LENGTH = 437 |
| 4584 | AT3G20430.1 Symbols: no symbol available: no full name available. Chr3: 7121480-7123074 |
| | FORWARD LENGTH = 265 |
| 4585 | AT3G20620.1 Symbols: no symbol available: no full name available. Chr3: 7198708-7199969 |
| | REVERSE LENGTH = 391 |
| 4586 | AT3G20810.2 Symbols: JMJ30, JMJD5: Jumonji C domain-containing protein 30, jumonji domain |
| | containing 5. Chr3: 7275814-7278144 FORWARD LENGTH = 429 |
| 4587 | AT3G20870.1 Symbols: ZTP29: zinc transporter 29. Chr3: 7309499-7312476 REVERSE LENGTH = 276 |
| 4588 | AT3G21250.2 Symbols: ATMRP6, ABCC8, MRP6: multidrug resistance-associated protein |
| | 6, ARABIDOPSIS THALIANA MULTIDRUG RESISTANCE-ASSOCIATED PROTEIN 6, ATP-binding |
| | cassette C8. Chr3: 7457668-7463261 REVERSE LENGTH = 1464 |
| 4589 | AT3G21295.1 Symbols: no symbol available: no full name available. Chr3: 7489635-7492296 |
| | FORWARD LENGTH = 645 |
| 4590 | AT3G21310.1 Symbols: no symbol available: no full name available. Chr3: 7497774-7499011 |
| | FORWARD LENGTH = 383 |
| 4591 | AT3G21700.3 Symbols: SGP2, ATSGP2.: Chr3: 7644581-7646190 FORWARD LENGTH = 292 |
| 4592 | AT3G22415.1 Symbols: no symbol available: no full name available. Chr3: 7941337-7941540 |
| | FORWARD LENGTH = 67 |
| 4593 | AT3G22550.1 Symbols: no symbol available: no full name available. Chr3: 7991827-7992805 |
| | REVERSE LENGTH = 267 |
| 4594 | AT3G22750.1 Symbols: no symbol available: no full name available. Chr3: 8037364-8039096 |
| | REVERSE LENGTH = 378 |
| 4595 | AT3G22970.1 Symbols: no symbol available: no full name available. Chr3: 8152592-8153881 |
| | FORWARD LENGTH = 370 |
| 4596 | AT3G23160.1 Symbols: no symbol available: no full name available. Chr3: 8260059-8261654 |
| | REVERSE LENGTH = 531 |
| 4597 | AT3G24070.1 Symbols: no symbol available: no full name available. Chr3: 8693543-8694331 |
| | FORWARD LENGTH = 262 |
| 4598 | AT3G24490.1 Symbols: no symbol available: no full name available. Chr3: 8911022-8912023 |
| | FORWARD LENGTH = 333 |
| 4599 | AT3G25570.1 Symbols: no symbol available: no full name available. Chr3: 9287413-9288462 |
| | REVERSE LENGTH = 349 |
| 4600 | AT3G25585.2 Symbols: AAPT2, ATAAPT2: aminoalcoholphosphotransferase. Chr3: 9295856-9298271 |
| | FORWARD LENGTH = 389 |
| 4601 | AT3G25590.1 Symbols: no symbol available: no full name available. Chr3: 9302271-9303542 |
| | FORWARD LENGTH = 423 |
| 4602 | AT3G25597.1 Symbols: no symbol available: no full name available. Chr3: 9306103-9306585 |
| | FORWARD LENGTH = 160 |
| 4603 | AT3G25840.1 Symbols: no symbol available: no full name available. Chr3: 9452993-9457446 |
| | REVERSE LENGTH = 935 |
| 4604 | AT3G26000.1 Symbols: RIFP1: RCAR3 INTERACTING F-BOX PROTEIN 1. Chr3: 9507042- |
| | 9508542 REVERSE LENGTH = 453 |
| 4605 | AT3G26085.2 Symbols: no symbol available: no full name available. Chr3: 9530842-9532397 |
| | FORWARD LENGTH = 322 |
| 4606 | AT3G26100.2 Symbols: no symbol available: no full name available. Chr3: 9538063-9540149 |
| | FORWARD LENGTH = 532 |
| 4607 | AT3G26230.1 Symbols: CYP71B24: “cytochrome P450, family 71, subfamily B, polypeptide 24”. |
| | Chr3: 9598200-9599892 REVERSE LENGTH = 498 |
| 4608 | AT3G26430.1 Symbols: no symbol available: no full name available. Chr3: 9674419-9675889 |
| | FORWARD LENGTH = 380 |
| 4609 | AT3G26440.1 Symbols: no symbol available: no full name available. Chr3: 9677418-9679681 |
| | FORWARD LENGTH = 396 |
| 4610 | AT3G26690.1 Symbols: ATNUDX13, ATNUDT13, NUDX13: nudix hydrolase homolog |
| | 13, ARABIDOPSIS THALIANA NUDIX HYDROLASE HOMOLOG 13. Chr3: 9804418-9805398 |
| | REVERSE LENGTH = 202 |
| 4611 | AT3G26744.1 Symbols: ICE1, SCRM, ATICE1: A. THALIANA INDUCER OF CBP EXPRESSION |
| | 1, INDUCER OF CBF EXPRESSION 1, SCREAM. Chr3: 9832953-9834790 REVERSE LENGTH = 494 |
| 4612 | AT3G26890.1 Symbols: no symbol available: no full name available. Chr3: 9907456-9910463 |
| | REVERSE LENGTH = 649 |
| 4613 | AT3G26910.2 Symbols: no symbol available: no full name available. Chr3: 9915304-9918511 |
| | REVERSE LENGTH = 614 |
| 4614 | AT3G26920.1 Symbols: no symbol available: no full name available. Chr3: 9921241-9922429 |
| | FORWARD LENGTH = 340 |
| 4615 | AT3G27260.1 Symbols: GTE8: global transcription factor group E8. Chr3: 10068411-10072403 |
| | FORWARD LENGTH = 813 |
| 4616 | AT3G27330.1 Symbols: no symbol available: no full name available. Chr3: 10116950-10120516 |
| | REVERSE LENGTH = 913 |
| 4617 | AT3G27350.2 Symbols: no symbol available: no full name available. Chr3: 10126238-10127896 |
| | FORWARD LENGTH = 319 |
| 4618 | AT3G27510.1 Symbols: no symbol available: no full name available. Chr3: 10188240-10189562 |
| | REVERSE LENGTH = 440 |
| 4619 | AT3G27560.1 Symbols: ATNI.: Chr3: 10210597-10212507 REVERSE LENGTH = 356 |
| 4620 | AT3G27940.1 Symbols: LBD26: LOB domain-containing protein 26. Chr3: 10375897-10376358 |
| | FORWARD LENGTH = 153 |
| 4621 | AT3G28690.2 Symbols: no symbol available: no full name available. Chr3: 10755412-10757494 |
| | FORWARD LENGTH = 476 |
| 4622 | AT3G28920.1 Symbols: ZHD9, AtHB34, HB34: ZINC FINGER HOMEODOMAIN 9, homeobox protein |
| | 34. Chr3: 10940598-10941536 REVERSE LENGTH = 312 |
| 4623 | AT3G29130.2 Symbols: no symbol available: no full name available. Chr3: 11102546-11103847 |
| | REVERSE LENGTH = 168 |
| 4624 | AT3G41762.1 Symbols: no symbol available: no full name available. Chr3: 14195832-14196464 |
| | REVERSE LENGTH = 109 |
| 4625 | AT3G42150.2 Symbols: no symbol available: no full name available. Chr3: 14310567-14311465 |
| | FORWARD LENGTH = 100 |
| 4626 | AT3G43240.1 Symbols: no symbol available: no full name available. Chr3: 15210032-15214440 |
| | REVERSE LENGTH = 747 |
| 4627 | AT3G44740.1 Symbols: no symbol available: no full name available. Chr3: 16296410-16297687 |
| | REVERSE LENGTH = 244 |
| 4628 | AT3G45090.1 Symbols: no symbol available: no full name available. Chr3: 16490752-16494452 |
| | REVERSE LENGTH = 717 |
| 4629 | AT3G45240.1 Symbols: ATSNAK2, GRIK1: geminivirus rep interacting kinase 1. Chr3: 16570774- |
| | 16572902 REVERSE LENGTH = 396 |
| 4630 | AT3G45870.1 Symbols: UMAMIT3: Usually multiple acids move in and out Transporters 3. |
| | Chr3: 16867246-16868838 FORWARD LENGTH = 385 |
| 4631 | AT3G45960.2 Symbols: ATEXPL3, EXLA3, ATEXLA3, ATHEXP BETA 2.3, EXPL3: expansin-like A3. |
| | Chr3: 16892826-16893789 FORWARD LENGTH = 263 |
| 4632 | AT3G46590.2 Symbols: TRP2, TRFL1, ATTRP2: TRF-like 1. Chr3: 17153642-17155946 FORWARD |
| | LENGTH = 553 |
| 4633 | AT3G46930.1 Symbols: Raf43: Raf-Like Mitogen-Activated Protein Kinase Kinase Kinase 43. |
| | Chr3: 17285931-17288032 FORWARD LENGTH = 480 |
| 4634 | AT3G47390.1 Symbols: PHS1, PyrR: pyrimidine reductase, PHOTOSENSITIVE 1. Chr3: 17462094- |
| | 17464655 FORWARD LENGTH = 599 |
| 4635 | AT3G47550.3 Symbols: no symbol available: no full name available. Chr3: 17523841-17525278 |
| | FORWARD LENGTH = 288 |
| 4636 | AT3G47680.1 Symbols: no symbol available: no full name available. Chr3: 17577483-17578391 |
| | REVERSE LENGTH = 302 |
| 4637 | AT3G47965.1 Symbols: no symbol available: no full name available. Chr3: 17708096-17708709 |
| | REVERSE LENGTH = 44 |
| 4638 | AT3G48060.1 Symbols: no symbol available: no full name available. Chr3: 17743234-17748372 |
| | REVERSE LENGTH = 1611 |
| 4639 | AT3G49050.1 Symbols: no symbol available: no full name available. Chr3: 18181498-18183613 |
| | FORWARD LENGTH = 477 |
| 4640 | AT3G49290.1 Symbols: ABIL2: ABL interactor-like protein 2. Chr3: 18273407-18275221 FORWARD |
| | LENGTH = 312 |
| 4641 | AT3G49430.1 Symbols: SR34a, SRp34a, At-SR34a: Serine/Arginine-Rich Protein Splicing Factor |
| | 34a, SER/ARG-rich protein 34A. Chr3: 18332668-18334829 FORWARD LENGTH = 300 |
| 4642 | AT3G49690.1 Symbols: RAX3, MYB84, ATMYB84, SKI2: MYB DOMAIN PROTEIN 84, myb domain |
| | protein 84, REGULATOR OF AXILLARY MERISTEMS3. Chr3: 18427941-18429100 FORWARD |
| | LENGTH = 310 |
| 4643 | AT3G49725.1 Symbols: no symbol available: no full name available. Chr3: 18442448-18445397 |
| | REVERSE LENGTH = 620 |
| 4644 | AT3G49810.1 Symbols: atPub30: U-box protein 30. Chr3: 18474936-18476282 REVERSE |
| | LENGTH = 448 |
| 4645 | AT3G49845.1 Symbols: WIH3: WINDHOSE 3. Chr3: 18487339-18487965 FORWARD LENGTH = 124 |
| 4646 | AT3G50700.1 Symbols: AtIDD2, IDD2: indeterminate(ID)-domain 2. Chr3: 18840945-18842829 |
| | FORWARD LENGTH = 452 |
| 4647 | AT3G50860.1 Symbols: no symbol available: no full name available. Chr3: 18902346-18903959 |
| | FORWARD LENGTH = 166 |
| 4648 | AT3G51040.1 Symbols: RTH: RTE1-homolog. Chr3: 18952281-18953060 REVERSE LENGTH = 231 |
| 4649 | AT3G51140.1 Symbols: no symbol available: no full name available. Chr3: 18998182-18999437 |
| | FORWARD LENGTH = 278 |
| 4650 | AT3G51180.1 Symbols: no symbol available: no full name available. Chr3: 19013720-19016042 |
| | FORWARD LENGTH = 521 |
| 4651 | AT3G51290.1 Symbols: APSR1: Altered Phosphate Starvation Response 1. Chr3: 19039980-19042437 |
| | FORWARD LENGTH = 634 |
| 4652 | AT3G51300.1 Symbols: ROP1AT, ATRAC11, ROP1, ARAC11, ATROP1: Arabidopsis RAC-like |
| | 11, RHO-related protein from plants 1, ARABIDOPSIS THALIANA RHO-RELATED PROTEIN FROM |
| | PLANTS 1. Chr3: 19043197-19044215 FORWARD LENGTH = 197 |
| 4653 | AT3G51370.1 Symbols: no symbol available: no full name available. Chr3: 19070054-19071975 |
| | FORWARD LENGTH = 379 |
| 4654 | AT3G51480.1 Symbols: GLR3.6, ATGLR3.6: glutamate receptor 3.6. Chr3: 19101256-19104681 |
| | FORWARD LENGTH = 903 |
| 4655 | AT3G51630.1 Symbols: ATWNK5, ZIK1, WNK5: with no lysine (K) kinase 5. Chr3: 19149487-19151924 |
| | FORWARD LENGTH = 549 |
| 4656 | AT3G51950.1 Symbols: no symbol available: no full name available. Chr3: 19278244-19280407 |
| | REVERSE LENGTH = 540 |
| 4657 | AT3G52170.1 Symbols: no symbol available: no full name available. Chr3: 19347039-19349099 |
| | REVERSE LENGTH = 499 |
| 4658 | AT3G52290.1 Symbols: IQD3: IQ-domain 3. Chr3: 19394441-19396196 FORWARD LENGTH = 430 |
| 4659 | AT3G52730.1 Symbols: no symbol available: no full name available. Chr3: 19543146-19544167 |
| | REVERSE LENGTH = 72 |
| 4660 | AT3G52880.2 Symbols: ATMDAR1, MDAR1: monodehydroascorbate reductase 1. Chr3: 19601477- |
| | 19604366 REVERSE LENGTH = 466 |
| 4661 | AT3G52890.1 Symbols: KIPK: KCBP-interacting protein kinase. Chr3: 19609150-19612032 |
| | FORWARD LENGTH = 934 |
| 4662 | AT3G52990.1 Symbols: no symbol available: no full name available. Chr3: 19649046-19652237 |
| | FORWARD LENGTH = 527 |
| 4663 | AT3G53270.1 Symbols: no symbol available: no full name available. Chr3: 19750406-19752168 |
| | REVERSE LENGTH = 280 |
| 4664 | AT3G53500.2 Symbols: RS2Z32, At-RS2Z, RSZ32: arginine/serine-rich zinc knuckle-containing protein |
| | 32. Chr3: 19834557-19836507 REVERSE LENGTH = 284 |
| 4665 | AT3G53670.1 Symbols: no symbol available: no full name available. Chr3: 19891104-19892214 |
| | FORWARD LENGTH = 203 |
| 4666 | AT3G53850.1 Symbols: CASPL5B2: CASP-like protein 5B2. Chr3: 19948262-19948994 REVERSE |
| | LENGTH = 154 |
| 4667 | AT3G54010.1 Symbols: DEII, PASI: PASTICCINO 1. Chr3: 20001042-20005063 FORWARD |
| | LENGTH = 635 |
| 4668 | AT3G54190.1 Symbols: no symbol available: no full name available. Chr3: 20061759-20063880 |
| | REVERSE LENGTH = 467 |
| 4669 | AT3G54220.1 Symbols: SGR1, SCR: SCARECROW, SHOOT GRAVITROPISM I. Chr3: 20070550- |
| | 20072625 FORWARD LENGTH = 653 |
| 4670 | AT3G54350.1 Symbols: emb1967: embryo defective 1967. Chr3: 20122298-20126031 REVERSE |
| | LENGTH = 702 |
| 4671 | AT3G54380.1 Symbols: SAC3C, AtSAC3C: yeast Sac3 homolog C. Chr3: 20133985-20136752 |
| | REVERSE LENGTH = 406 |
| 4672 | AT3G55020.1 Symbols: no symbol available: no full name available. Chr3: 20389178-20394713 |
| | REVERSE LENGTH = 819 |
| 4673 | AT3G55050.1 Symbols: no symbol available: no full name available. Chr3: 20400669-20401922 |
| | REVERSE LENGTH = 384 |
| 4674 | AT3G55080.1 Symbols: no symbol available: no full name available. Chr3: 20411476-20415830 |
| | REVERSE LENGTH = 463 |
| 4675 | AT3G55140.1 Symbols: no symbol available: no full name available. Chr3: 20438819-20440225 |
| | FORWARD LENGTH = 331 |
| 4676 | AT3G55480.2 Symbols: PAT2, WATI, AP-3 beta: beta-subunit of adaptor protein complex 3, protein |
| | affected traf?cking 2, WEAK ACID TOLERANT 1. Chr3: 20566372-20571171 REVERSE |
| | LENGTH = 1115 |
| 4677 | AT3G55560.1 Symbols: AGF2, AHL15: AT-hook protein of GA feedback 2, AT-hook motif nuclear- |
| | localized protein 15. Chr3: 20604904-20605836 REVERSE LENGTH = 310 |
| 4678 | AT3G55820.1 Symbols: no symbol available: no full name available. Chr3: 20714041-20714655 |
| | FORWARD LENGTH = 204 |
| 4679 | AT3G55850.2 Symbols: LAF3 ISF2, LAF3 ISF1, LAF3: LAF3 ISOFORM 2, LONG AFTER FAR-RED 3 |
| | ISOFORM 1, LONG AFTER FAR-RED 3. Chr3: 20721866-20724721 REVERSE LENGTH = 461 |
| 4680 | AT3G55860.1 Symbols: no symbol available: no full name available. Chr3: 20727836-20728962 |
| | REVERSE LENGTH = 154 |
| 4681 | AT3G56130.1 Symbols: BLP3: BCCP-Like Protein 3. Chr3: 20826852-20829007 FORWARD |
| | LENGTH = 281 |
| 4682 | AT3G56270.1 Symbols: no symbol available: no full name available. Chr3: 20870220-20871854 |
| | FORWARD LENGTH = 446 |
| 4683 | AT3G56580.2 Symbols: RZF1, AtRZF1: RING Zinc Finger 1. Chr3: 20962563-20963525 FORWARD |
| | LENGTH = 320 |
| 4684 | AT3G56850.1 Symbols: DPBF3, AREB3: ABA-responsive element binding protein 3. Chr3: 21046554- |
| | 21047894 REVERSE LENGTH = 297 |
| 4685 | AT3G57630.1 Symbols: no symbol available: no full name available. Chr3: 21339543-21343080 |
| | REVERSE LENGTH = 793 |
| 4686 | AT3G57790.1 Symbols: no symbol available: no full name available. Chr3: 21405387-21407088 |
| | REVERSE LENGTH = 490 |
| 4687 | AT3G58030.1 Symbols: no symbol available: no full name available. Chr3: 21485527-21486837 |
| | FORWARD LENGTH = 436 |
| 4688 | AT3G58510.1 Symbols: no symbol available: no full name available. Chr3: 21640608-21643464 |
| | FORWARD LENGTH = 612 |
| 4689 | AT3G58520.1 Symbols: no symbol available: no full name available. Chr3: 21643921-21645277 |
| | REVERSE LENGTH = 418 |
| 4690 | AT3G58560.1 Symbols: AtCCR4a.: Chr3: 21650880-21653896 REVERSE LENGTH = 602 |
| 4691 | AT3G58650.1 Symbols: TRM7: TON1 Recruiting Motif 7. Chr3: 21696349-21699219 REVERSE |
| | LENGTH = 820 |
| 4692 | AT3G58670.1 Symbols: PCO5: plant cysteine oxidase 5. Chr3: 21703693-21705314 REVERSE |
| | LENGTH = 242 |
| 4693 | AT3G59470.1 Symbols: FRF1: FARI-RELATED SEQUENCES-RELATED FACTOR1. |
| | Chr3: 21979099-21980097 REVERSE LENGTH = 251 |
| 4694 | AT3G59570.1 Symbols: no symbol available: no full name available. Chr3: 22001030-22005402 |
| | REVERSE LENGTH = 720 |
| 4695 | AT3G59770.3 Symbols: AtSAC9, SAC9: ARABIDOPSIS THALIANA SUPPRESSOR OF ACTIN |
| | 9, SUPPRESSOR OF ACTIN 9. Chr3: 22079281-22085674 REVERSE LENGTH = 1646 |
| 4696 | AT3G60240.4 Symbols: EIF4G, CUM2: eukaryotic translation initiation factor 4G, CUCUMOVIRUS |
| | MULTIPLICATION 2. Chr3: 22261842-22268295 FORWARD LENGTH = 1727 |
| 4697 | AT3G60540.1 Symbols: no symbol available: no full name available. Chr3: 22375132-22375377 |
| | REVERSE LENGTH = 81 |
| 4698 | AT3G60800.1 Symbols: PAT14: protein acyltransferase 14. Chr3: 22467486-22469273 REVERSE |
| | LENGTH = 307 |
| 4699 | AT3G61160.2 Symbols: no symbol available: no full name available. Chr3: 22636209-22638593 |
| | FORWARD LENGTH = 438 |
| 4700 | AT3G61250.1 Symbols: LMI2, MYB17, AtMYB17: LATE MERISTEM IDENTITY2, myb domain |
| | protein 17. Chr3: 22671306-22672551 FORWARD LENGTH = 299 |
| 4701 | AT3G61310.1 Symbols: AHL11: AT-hook motif nuclear localized protein 11. Chr3: 22690799-22692445 |
| | REVERSE LENGTH = 354 |
| 4702 | AT3G61530.1 Symbols: PANB2.: Chr3: 22771692-22773313 REVERSE LENGTH = 354 |
| 4703 | AT3G61550.1 Symbols: no symbol available: no full name available. Chr3: 22776444-22777082 |
| | FORWARD LENGTH = 212 |
| 4704 | AT3G61590.1 Symbols: HS, HWS: HAWAIIAN SKIRT. Chr3: 22792914-22794149 FORWARD |
| | LENGTH = 411 |
| 4705 | AT3G61620.1 Symbols: RRP41.: Chr3: 22801382-22802910 REVERSE LENGTH = 241 |
| 4706 | AT3G61680.1 Symbols: no symbol available: no full name available. Chr3: 22824630-22826926 |
| | FORWARD LENGTH = 649 |
| 4707 | AT3G61830.1 Symbols: ARF18: auxin response factor 18. Chr3: 22888171-22891179 FORWARD |
| | LENGTH = 602 |
| 4708 | AT3G61850.4 Symbols: DAG1: dof affecting germination 1. Chr3: 22895495-22897150 FORWARD |
| | LENGTH = 324 |
| 4709 | AT3G62020.1 Symbols: GLP10: germin-like protein 10. Chr3: 22971443-22972192 REVERSE |
| | LENGTH = 220 |
| 4710 | AT3G62190.1 Symbols: no symbol available: no full name available. Chr3: 23021146-23022614 |
| | FORWARD LENGTH = 138 |
| 4711 | AT3G62270.1 Symbols: BOR2: REQUIRES HIGH BORON 2. Chr3: 23042528-23045633 REVERSE |
| | LENGTH = 703 |
| 4712 | AT3G62400.2 Symbols: no symbol available: no full name available. Chr3: 23090413-23090673 |
| | FORWARD LENGTH = 86 |
| 4713 | AT3G62420.1 Symbols: ATBZIP53, BZIP53: basic region/leucine zipper motif 53. Chr3: 23091844- |
| | 23092284 REVERSE LENGTH = 146 |
| 4714 | AT3G62660.1 Symbols: GATL7: galacturonosyltransferase-like 7. Chr3: 23173419-23174504 |
| | FORWARD LENGTH = 361 |
| 4715 | AT3G63050.1 Symbols: no symbol available: no full name available. Chr3: 23297950-23298372 |
| | REVERSE LENGTH = 140 |
| 4716 | AT3G63080.1 Symbols: MEE42, GPX5, ATGPX5: maternal effect embryo arrest 42, glutathione |
| | peroxidase 5. Chr3: 23310161-23311200 FORWARD LENGTH = 173 |
| 4717 | AT3G63130.1 Symbols: ATRANGAP1, RANGAP1: RAN GTPase activating protein 1, RAN GTPASE- |
| | ACTIVATING PROTEIN 1. Chr3: 23325108-23326715 FORWARD LENGTH = 535 |
| 4718 | AT3G63210.1 Symbols: MARD1: MEDIATOR OF ABA-REGULATED DORMANCY 1. |
| | Chr3: 23354019-23354906 REVERSE LENGTH = 263 |
| 4719 | AT3G63240.1 Symbols: no symbol available: no full name available. Chr3: 23364411-23366876 |
| | REVERSE LENGTH = 547 |
| 4720 | AT3G63250.1 Symbols: HMT-2, ATHMT-2, HMT2: homocysteine methyltransferase |
| | 2, HOMOCYSTEINE METHYLTRANSFERASE-2. Chr3: 23370575-23372587 REVERSE |
| | LENGTH = 333 |
| 4721 | AT3G63450.3 Symbols: no symbol available: no full name available. Chr3: 23427191-23428864 |
| | REVERSE LENGTH = 406 |
| 4722 | AT3G66654.1 Symbols: no symbol available: no full name available. Chr3: 2088694-2090297 |
| | FORWARD LENGTH = 236 |
| 4723 | AT4G00110.1 Symbols: GAE3: UDP-D-glucuronate 4-epimerase 3. Chr4: 38702-39994 REVERSE |
| | LENGTH = 430 |
| 4724 | AT4G00130.1 Symbols: no symbol available: no full name available. Chr4: 48414-49068 FORWARD |
| | LENGTH = 194 |
| 4725 | AT4G00180.1 Symbols: YAB3: YABBY3. Chr4: 72804-75089 REVERSE LENGTH = 240 |
| 4726 | AT4G00390.1 Symbols: no symbol available: no full name available. Chr4: 171650-172744 REVERSE |
| | LENGTH = 364 |
| 4727 | AT4G00440.2 Symbols: TRM15: TON1 Recruiting Motif 15. Chr4: 195176-198225 FORWARD |
| | LENGTH = 835 |
| 4728 | AT4G00500.1 Symbols: no symbol available: no full name available. Chr4: 225214-226978 REVERSE |
| | LENGTH = 460 |
| 4729 | AT4G00550.1 Symbols: DGD2: digalactosyl diacylglycerol deficient 2. Chr4: 238154-240019 |
| | REVERSE LENGTH = 473 |
| 4730 | AT4G00710.1 Symbols: BSK3: brassinosteroid-signaling kinase 3. Chr4: 290807-293096 FORWARD |
| | LENGTH = 489 |
| 4731 | AT4G00730.1 Symbols: AHDP, ANL2: ANTHOCYANINLESS 2, ARABIDOPSIS THALIANA |
| | HOMEODOMAIN PROTEIN. Chr4: 299741-304103 REVERSE LENGTH = 802 |
| 4732 | AT4G00955.1 Symbols: no symbol available: no full name available. Chr4: 412218-412976 FORWARD |
| | LENGTH = 252 |
| 4733 | AT4G01090.1 Symbols: no symbol available: no full name available. Chr4: 470834-473248 REVERSE |
| | LENGTH = 714 |
| 4734 | AT4G01210.1 Symbols: no symbol available: no full name available. Chr4: 507738-512362 REVERSE |
| | LENGTH = 1031 |
| 4735 | AT4G01290.1 Symbols: no symbol available: no full name available. Chr4: 538213-543282 REVERSE |
| | LENGTH = 991 |
| 4736 | AT4G02010.1 Symbols: no symbol available: no full name available. Chr4: 881457-885222 FORWARD |
| | LENGTH = 725 |
| 4737 | AT4G02140.1 Symbols: no symbol available: no full name available. Chr4: 949810-950240 REVERSE |
| | LENGTH = 112 |
| 4738 | AT4G02170.1 Symbols: no symbol available: no full name available. Chr4: 958126-958641 FORWARD |
| | LENGTH = 171 |
| 4739 | AT4G02550.3 Symbols: no symbol available: no full name available. Chr4: 1120622-1121674 |
| | REVERSE LENGTH = 322 |
| 4740 | AT4G02570.1 Symbols: ICU13, AXR6, ATCUL1, CUL1: cullin 1, AUXIN RESISTANT 6, INCURVATA |
| | 13. Chr4: 1129315-1133435 FORWARD LENGTH = 738 |
| 4741 | AT4G02710.1 Symbols: NET1C: Networked 1C. Chr4: 1193516-1197061 REVERSE LENGTH = 1111 |
| 4742 | AT4G02760.3 Symbols: no symbol available: no full name available. Chr4: 1224336-1227857 |
| | REVERSE LENGTH = 551 |
| 4743 | AT4G03000.1 Symbols: no symbol available: no full name available. Chr4: 1324602-1327348 |
| | FORWARD LENGTH = 814 |
| 4744 | AT4G03090.1 Symbols: NDX, AtNDX: Nodulin Homeobox. Chr4: 1366495-1370937 REVERSE |
| | LENGTH = 913 |
| 4745 | AT4G03200.1 Symbols: no symbol available: no full name available. Chr4: 1408296-1412566 |
| | FORWARD LENGTH = 818 |
| 4746 | AT4G03205.2 Symbols: hemf2.: Chr4: 1412814-1413703 FORWARD LENGTH = 240 |
| 4747 | AT4G03260.1 Symbols: no symbol available: no full name available. Chr4: 1429092-1431431 |
| | FORWARD LENGTH = 677 |
| 4748 | AT4G03280.1 Symbols: PGR1, PETC: PROTON GRADIENT REGULATION 1, photosynthetic electron |
| | transfer C. Chr4: 1440314-1441717 FORWARD LENGTH = 229 |
| 4749 | AT4G03410.2 Symbols: no symbol available: no full name available. Chr4: 1501907-1503503 |
| | FORWARD LENGTH = 361 |
| 4750 | AT4G03500.1 Symbols: no symbol available: no full name available. Chr4: 1553453-1556571 |
| | FORWARD LENGTH = 652 |
| 4751 | AT4G03510.1 Symbols: ATRMA1, RMA1: RING membrane-anchor 1. Chr4: 1557905-1558654 |
| | REVERSE LENGTH = 249 |
| 4752 | AT4G04340.2 Symbols: oscal: reduced hyperosmolality, induced Ca2+ increase 1 (oscal). |
| | Chr4: 2123235-2126624 FORWARD LENGTH = 772 |
| 4753 | AT4G04740.2 Symbols: CPK23, ATCPK23: calcium-dependent protein kinase 23. Chr4: 2404883- |
| | 2408493 REVERSE LENGTH = 533 |
| 4754 | AT4G04890.1 Symbols: PDF2: protodermal factor 2. Chr4: 2476970-2480090 REVERSE LENGTH = 743 |
| 4755 | AT4G06634.1 Symbols: AtYY1, YY1: Yin Yang 1. Chr4: 3764499-3766439 REVERSE LENGTH = 387 |
| 4756 | AT4G07990.1 Symbols: no symbol available: no full name available. Chr4: 4825650-4828937 |
| | REVERSE LENGTH = 230 |
| 4757 | AT4G08700.1 Symbols: PUP13, ATPUP13: purine permease 13. Chr4: 5565998-5567286 REVERSE |
| | LENGTH = 361 |
| 4758 | AT4G08980.1 Symbols: FBW2: F-BOX WITH WD-40 2. Chr4: 5758993-5760108 FORWARD |
| | LENGTH = 317 |
| 4759 | AT4G09640.1 Symbols: no symbol available: no full name available. Chr4: 6088433-6090604 |
| | REVERSE LENGTH = 386 |
| 4760 | AT4G10390.1 Symbols: no symbol available: no full name available. Chr4: 6441949-6443161 |
| | REVERSE LENGTH = 342 |
| 4761 | AT4G10955.1 Symbols: no symbol available: no full name available. Chr4: 6713778-6715372 |
| | REVERSE LENGTH = 350 |
| 4762 | AT4G11440.1 Symbols: no symbol available: no full name available. Chr4: 6955850-6958553 |
| | FORWARD LENGTH = 628 |
| 4763 | AT4G11450.1 Symbols: no symbol available: no full name available. Chr4: 6959065-6961223 |
| | REVERSE LENGTH = 694 |
| 4764 | AT4G11570.1 Symbols: no symbol available: no full name available. Chr4: 7004743-7005864 |
| | FORWARD LENGTH = 373 |
| 4765 | AT4G11800.1 Symbols: no symbol available: no full name available. Chr4: 7093666-7098519 |
| | REVERSE LENGTH = 1013 |
| 4766 | AT4G11830.2 Symbols: PLDGAMMA2: phospholipase D gamma 2. Chr4: 7115985-7119683 |
| | REVERSE LENGTH = 856 |
| 4767 | AT4G11960.1 Symbols: PGRL1B: PGR5-like B. Chr4: 7175340-7177709 REVERSE LENGTH = 313 |
| 4768 | AT4G12040.1 Symbols: AtSAP7, SAP7: stress-associated protein 7. Chr4: 7215341-7215868 |
| | FORWARD LENGTH = 175 |
| 4769 | AT4G12120.1 Symbols: SEC1B, ATSEC1B.: Chr4: 7256687-7260914 REVERSE LENGTH = 662 |
| 4770 | AT4G12420.1 Symbols: SKU5.: Chr4: 7349941-7352868 REVERSE LENGTH = 587 |
| 4771 | AT4G12750.1 Symbols: no symbol available: no full name available. Chr4: 7498009-7503920 |
| | REVERSE LENGTH = 1117 |
| 4772 | AT4G12990.1 Symbols: no symbol available: no full name available. Chr4: 7595044-7595334 |
| | REVERSE LENGTH = 96 |
| 4773 | AT4G13020.3 Symbols: MHK.: Chr4: 7604015-7606812 FORWARD LENGTH = 444 |
| 4774 | AT4G13100.2 Symbols: no symbol available: no full name available. Chr4: 7636547-7639037 |
| | REVERSE LENGTH = 265 |
| 4775 | AT4G13260.1 Symbols: AtYUC2, YUC2: YUCCA2. Chr4: 7721840-7723616 REVERSE LENGTH = 415 |
| 4776 | AT4G13345.1 Symbols: MEE55: maternal effect embryo arrest 55. Chr4: 7767292-7769426 FORWARD |
| | LENGTH = 394 |
| 4777 | AT4G13550.1 Symbols: no symbol available: no full name available. Chr4: 7871251-7876877 |
| | REVERSE LENGTH = 822 |
| 4778 | AT4G14350.1 Symbols: no symbol available: no full name available. Chr4: 8256449-8259934 |
| | REVERSE LENGTH = 551 |
| 4779 | AT4G14520.1 Symbols: no symbol available: no full name available. Chr4: 8342471-8343073 |
| | FORWARD LENGTH = 200 |
| 4780 | AT4G14620.1 Symbols: no symbol available: no full name available. Chr4: 8389246-8390448 |
| | REVERSE LENGTH = 341 |
| 4781 | AT4G14710.5 Symbols: ATARD2.: Chr4: 8424897-8426174 REVERSE LENGTH = 201 |
| 4782 | AT4G14850.1 Symbols: LOI1, MEF11: lovastatin insensitive 1, mitochondrial RNA editing factor 11. |
| | Chr4: 8513947-8516275 FORWARD LENGTH = 684 |
| 4783 | AT4G14880.1 Symbols: OASA1, OLD3, CYTACS1, ATCYS-3A: ONSET OF LEAF DEATH 3,O- |
| | acetylserine (thiol) lyase (OAS-TL) isoform A1. Chr4: 8518209-8520050 REVERSE LENGTH = 322 |
| 4784 | AT4G15010.1 Symbols: no symbol available: no full name available. Chr4: 8573125-8574864 |
| | REVERSE LENGTH = 378 |
| 4785 | AT4G15080.1 Symbols: no symbol available: no full name available. Chr4: 8609085-8612229 |
| | REVERSE LENGTH = 718 |
| 4786 | AT4G15530.5 Symbols: PPDK: pyruvate orthophosphate dikinase. Chr4: 8864828-8870748 REVERSE |
| | LENGTH = 963 |
| 4787 | AT4G16070.1 Symbols: no symbol available: no full name available. Chr4: 9096808-9100596 |
| | REVERSE LENGTH = 654 |
| 4788 | AT4G16280.2 Symbols: FCA.: Chr4: 9207164-9214412 REVERSE LENGTH = 747 |
| 4789 | AT4G16360.1 Symbols: KINbeta2: 5′-AMP-activated protein kinase beta-2 subunit, putative: |
| | Chr4: 9245571-9246833 FORWARD LENGTH = 289 |
| 4790 | AT4G16520.1 Symbols: ATG8F: autophagy 8f. Chr4: 9306882-9308113 REVERSE LENGTH = 121 |
| 4791 | AT4G16765.1 Symbols: no symbol available: no full name available. Chr4: 9429974-9431791 |
| | REVERSE LENGTH = 247 |
| 4792 | AT4G16845.1 Symbols: VRN2: REDUCED VERNALIZATION RESPONSE 2. Chr4: 9476708- |
| | 9479725 FORWARD LENGTH = 440 |
| 4793 | AT4G16940.1 Symbols: no symbol available: no full name available. Chr4: 9533149-9537510 |
| | REVERSE LENGTH = 1147 |
| 4794 | AT4G16990.2 Symbols: RLM3: RESISTANCE TO LEPTOSPHAERIA MACULANS 3. |
| | Chr4: 9560155-9565225 FORWARD LENGTH = 796 |
| 4795 | AT4G17020.3 Symbols: no symbol available: no full name available. Chr4: 9577973-9581082 |
| | REVERSE LENGTH = 482 |
| 4796 | AT4G17140.3 Symbols: no symbol available: no full name available. Chr4: 9613617-9636618 |
| | REVERSE LENGTH = 4219 |
| 4797 | AT4G17410.3 Symbols: no symbol available: no full name available. Chr4: 9716360-9721642 |
| | FORWARD LENGTH = 827 |
| 4798 | AT4G18020.1 Symbols: PRR2, APRR2: PSEUDO-RESPONSE REGULATOR 2. Chr4: 10003738- |
| | 10006682 REVERSE LENGTH = 535 |
| 4799 | AT4G18880.1 Symbols: AT-HSFA4A, HSF A4A: heat shock transcription factor A4A, ARABIDOPSIS |
| | THALIANA HEAT SHOCK TRANSCRIPTION FACTOR A4A. Chr4: 10347769-10349051 REVERSE |
| | LENGTH = 401 |
| 4800 | AT4G19110.2 Symbols: no symbol available: no full name available. Chr4: 10454770-10457468 |
| | REVERSE LENGTH = 464 |
| 4801 | AT4G19160.2 Symbols: no symbol available: no full name available. Chr4: 10477772-10479947 |
| | FORWARD LENGTH = 453 |
| 4802 | AT4G19550.2 Symbols: no symbol available: no full name available. Chr4: 10659414-10660873 |
| | REVERSE LENGTH = 223 |
| 4803 | AT4G19645.1 Symbols: no symbol available: no full name available. Chr4: 10689941-10691298 |
| | REVERSE LENGTH = 268 |
| 4804 | AT4G20325.1 Symbols: no symbol available: no full name available. Chr4: 10980124-10982426 |
| | FORWARD LENGTH = 277 |
| 4805 | AT4G21060.1 Symbols: AtGALT2, GALT2: AGP galactosyltransferase 2. Chr4: 11240730-11244860 |
| | FORWARD LENGTH = 741 |
| 4806 | AT4G21270.1 Symbols: KATA, KATAP, ATK1: KINESIN-LIKE PROTEIN IN ARABIDOPSIS |
| | THALIANA A, KINESIN-LIKE PROTEIN IN ARABIDOPSIS THALIANA A PROTEIN, kinesin 1. |
| | Chr4: 11329579-11333884 REVERSE LENGTH = 793 |
| 4807 | AT4G21720.1 Symbols: no symbol available: no full name available. Chr4: 11542561-11544274 |
| | FORWARD LENGTH = 139 |
| 4808 | AT4G21750.1 Symbols: ATML1: MERISTEM LAYER 1. Chr4: 11556965-11560243 FORWARD |
| | LENGTH = 762 |
| 4809 | AT4G21850.1 Symbols: MSRB9, ATMSRB9: methionine sulfoxide reductase B9. Chr4: 11591378- |
| | 11592138 REVERSE LENGTH = 143 |
| 4810 | AT4G22140.1 Symbols: EBS: EARLY BOLTING IN SHORT DAYS. Chr4: 11728556-11730230 |
| | REVERSE LENGTH = 234 |
| 4811 | AT4G22150.1 Symbols: PUX3: plant UBX domain-containing protein 3. Chr4: 11731272-11732605 |
| | REVERSE LENGTH = 302 |
| 4812 | AT4G22285.1 Symbols: no symbol available: no full name available. Chr4: 11779063-11781898 |
| | REVERSE LENGTH = 541 |
| 4813 | AT4G22305.1 Symbols: no symbol available: no full name available. Chr4: 11789546-11791055 |
| | REVERSE LENGTH = 228 |
| 4814 | AT4G22810.1 Symbols: AHL24: AT-hook motif nuclear localized protein 24. Chr4: 11984432-11985406 |
| | FORWARD LENGTH = 324 |
| 4815 | AT4G22860.1 Symbols: no symbol available: no full name available. Chr4: 11997735-12001281 |
| | FORWARD LENGTH = 509 |
| 4816 | AT4G22970.1 Symbols: RSW4, ESP, AESP: EXTRA SPINDLE POLES, RADIALLY SWOLLEN |
| | 4, homolog of separase. Chr4: 12033703-12043572 REVERSE LENGTH = 2180 |
| 4817 | AT4G22990.2 Symbols: PHT5 3: Chr4: 12048240-12050984 REVERSE LENGTH = 700 |
| 4818 | AT4G23000.1 Symbols: no symbol available: no full name available. Chr4: 12055241-12059647 |
| | FORWARD LENGTH = 919 |
| 4819 | AT4G23020.2 Symbols: TRM11: TON1 Recruiting Motif 11. Chr4: 12066331-12068442 REVERSE |
| | LENGTH = 452 |
| 4820 | AT4G23420.3 Symbols: no symbol available: no full name available. Chr4: 12226060-12228562 |
| | FORWARD LENGTH = 333 |
| 4821 | AT4G23470.1 Symbols: no symbol available: no full name available. Chr4: 12249289-12251079 |
| | FORWARD LENGTH = 255 |
| 4822 | AT4G23740.1 Symbols: no symbol available: no full name available. Chr4: 12367063-12369159 |
| | FORWARD LENGTH = 638 |
| 4823 | AT4G23770.1 Symbols: no symbol available: no full name available. Chr4: 12383514-12384050 |
| | REVERSE LENGTH = 138 |
| 4824 | AT4G23860.1 Symbols: no symbol available: no full name available. Chr4: 12409336-12411336 |
| | FORWARD LENGTH = 452 |
| 4825 | AT4G24480.1 Symbols: no symbol available: no full name available. Chr4: 12650410-12654755 |
| | FORWARD LENGTH = 956 |
| 4826 | AT4G24670.1 Symbols: TAR2: tryptophan aminotransferase related 2. Chr4: 12727940-12730694 |
| | REVERSE LENGTH = 440 |
| 4827 | AT4G24960.1 Symbols: HVA22D, ATHVA22D: ARABIDOPSIS THALIANA HVA22 HOMOLOGUE |
| | D, HVA22 homologue D. Chr4: 12828060-12828982 FORWARD LENGTH = 135 |
| 4828 | AT4G25100.1 Symbols: FSD1, ATFSDI: ARABIDOPSIS FE SUPEROXIDE DISMUTASE 1, Fe |
| | superoxide dismutase 1. Chr4: 12884649-12886501 REVERSE LENGTH = 212 |
| 4829 | AT4G25320.1 Symbols: AHL3: AT-HOOK MOTIF NUCLEAR LOCALIZED PROTEIN 3. |
| | Chr4: 12954488-12956342 FORWARD LENGTH = 404 |
| 4830 | AT4G25390.1 Symbols: no symbol available: no full name available. Chr4: 12977491-12979446 |
| | FORWARD LENGTH = 651 |
| 4831 | AT4G25670.1 Symbols: no symbol available: no full name available. Chr4: 13085431-13085997 |
| | REVERSE LENGTH = 188 |
| 4832 | AT4G25690.1 Symbols: no symbol available: no full name available. Chr4: 13090421-13090996 |
| | REVERSE LENGTH = 191 |
| 4833 | AT4G26100.1 Symbols: CKL1, CK1: casein kinase 1. Chr4: 13227885-13230508 REVERSE |
| | LENGTH = 450 |
| 4834 | AT4G26270.1 Symbols: PFK3: phosphofructokinase 3. Chr4: 13301094-13304030 REVERSE |
| | LENGTH = 489 |
| 4835 | AT4G26320.1 Symbols: AGP13: arabinogalactan protein 13. Chr4: 13317235-13317414 REVERSE |
| | LENGTH = 59 |
| 4836 | AT4G26520.1 Symbols: FBA7, AtFBA7: fructose-bisphosphate aldolase 7. Chr4: 13388676-13390191 |
| | FORWARD LENGTH = 358 |
| 4837 | AT4G26650.1 Symbols: no symbol available: no full name available. Chr4: 13445552-13447592 |
| | FORWARD LENGTH = 455 |
| 4838 | AT4G26850.1 Symbols: GGP, VTC2: vitamin c defective 2, GDP-1-galactose phosphorylase. |
| | Chr4: 13499262-13501145 REVERSE LENGTH = 442 |
| 4839 | AT4G27010.2 Symbols: EMB2788: EMBRYO DEFECTIVE 2788. Chr4: 13558129-13568296 |
| | FORWARD LENGTH = 2574 |
| 4840 | AT4G27040.1 Symbols: VPS22.: Chr4: 13573061-13574576 REVERSE LENGTH = 250 |
| 4841 | AT4G27050.1 Symbols: no symbol available: no full name available. Chr4: 13575884-13577429 |
| | REVERSE LENGTH = 453 |
| 4842 | AT4G27080.2 Symbols: PDIL5-4, ATPDI7, PDI7, ATPDIL5-4: PDI-like 5-4, PROTEIN DISULFIDE |
| | ISOMERASE 7, ARABIDOPSIS THALIANA PROTEIN DISULFIDE ISOMERASE 7. Chr4: 13589156- |
| | 13593335 FORWARD LENGTH = 532 |
| 4843 | AT4G27610.1 Symbols: no symbol available: no full name available. Chr4: 13785648-13787332 |
| | REVERSE LENGTH = 334 |
| 4844 | AT4G27840.1 Symbols: no symbol available: no full name available. Chr4: 13865268-13866050 |
| | REVERSE LENGTH = 260 |
| 4845 | AT4G28040.1 Symbols: UMAMIT33: Usually multiple acids move in and out Transporters 33. |
| | Chr4: 13940881-13942201 FORWARD LENGTH = 359 |
| 4846 | AT4G28260.1 Symbols: no symbol available: no full name available. Chr4: 14005048-14006762 |
| | FORWARD LENGTH = 516 |
| 4847 | AT4G28300.1 Symbols: no symbol available: no full name available. Chr4: 14014860-14016823 |
| | FORWARD LENGTH = 496 |
| 4848 | AT4G28480.1 Symbols: no symbol available: no full name available. Chr4: 14073310-14075091 |
| | FORWARD LENGTH = 348 |
| 4849 | AT4G29160.1 Symbols: SNF7.1.: Chr4: 14381019-14382342 FORWARD LENGTH = 219 |
| 4850 | AT4G30020.1 Symbols: no symbol available: no full name available. Chr4: 14678251-14681762 |
| | FORWARD LENGTH = 816 |
| 4851 | AT4G30160.2 Symbols: ATVLN4, VLN4: villin 4. Chr4: 14754528-14759511 FORWARD |
| | LENGTH-983 |
| 4852 | AT4G30630.1 Symbols: no symbol available: no full name available. Chr4: 14951048-14952159 |
| | FORWARD LENGTH = 237 |
| 4853 | AT4G30710.1 Symbols: QWRF8, AUG8: QWRF domain containing 8, augmin subunit 8. |
| | Chr4: 14965538-14967881 REVERSE LENGTH-644 |
| 4854 | AT4G30900.1 Symbols: no symbol available: no full name available. Chr4: 15040021-15042203 |
| | FORWARD LENGTH = 316 |
| 4855 | AT4G30960.1 Symbols: ATCIPK6, CIPK6, SNRK3.14, SIP3: SNF1-RELATED PROTEIN KINASE |
| | 3.14, SOS3-interacting protein 3, CBL-INTERACTING PROTEIN KINASE 6. Chr4: 15067400-15068725 |
| | FORWARD LENGTH = 441 |
| 4856 | AT4G31820.1 Symbols: ENP, MAB4, NPY1: MACCHI-BOU 4, NAKED PINS IN YUC MUTANTS |
| | 1, ENHANCER OF PINOID. Chr4: 15390788-15393627 REVERSE LENGTH = 571 |
| 4857 | AT4G31880.1 Symbols: AtPDS5C, PDS5C.: Chr4: 15419435-15423939 REVERSE LENGTH = 873 |
| 4858 | AT4G31990.3 Symbols: AAT3, ATAAT1, ASP5: aspartate aminotransferase 5, ASPARTATE |
| | AMINOTRANSFERASE DEFICIENT 3. Chr4: 15470876-15473521 REVERSE LENGTH = 462 |
| 4859 | AT4G32300.1 Symbols: SD2-5: S-domain-2 5. Chr4: 15599970-15602435 FORWARD LENGTH = 821 |
| 4860 | AT4G32850.8 Symbols: nPAP, PAP(IV), PAPS4: poly(A) polymerase 4, poly(A) polymerase IV, nuclear |
| | poly(a) polymerase. Chr4: 15850134-15854443 FORWARD LENGTH = 765 |
| 4861 | AT4G32980.1 Symbols: ATH1: homeobox gene 1. Chr4: 15914865-15916873 REVERSE |
| | LENGTH = 473 |
| 4862 | AT4G33050.2 Symbols: AtIQM1, IQM1, EDA39: IQ-motif protein 1, embryo sac development arrest 39. |
| | Chr4: 15944604-15946736 REVERSE LENGTH = 526 |
| 4863 | AT4G33160.1 Symbols: no symbol available: no full name available. Chr4: 15994160-15995533 |
| | FORWARD LENGTH = 457 |
| 4864 | AT4G33240.1 Symbols: FAB1A: FORMS APLOID AND BINUCLEATE CELLS 1A. Chr4: 16029504- |
| | 16037278 REVERSE LENGTH = 1757 |
| 4865 | AT4G33950.1 Symbols: SNRK2.6, SRK2E, ATOST1, P44, SNRK2-6, OST1: SNF1-RELATED PROTEIN |
| | KINASE 2.6, OPEN STOMATA 1, SUCROSE NONFERMENTING 1-RELATED PROTEIN KINASE 2- |
| | 6. Chr4: 16272364-16274657 FORWARD LENGTH = 362 |
| 4866 | AT4G34160.1 Symbols: CYCD3, CYCD3_1: CYCLIN D3_1. Chr4: 16357903-16359304 FORWARD |
| | LENGTH = 376 |
| 4867 | AT4G34420.1 Symbols: no symbol available: no full name available. Chr4: 16458430-16460526 |
| | FORWARD LENGTH = 342 |
| 4868 | AT4G34460.1 Symbols: ELK4, ATAGB1, AGB1: GTP binding protein beta 1, ERECTA-LIKE 4. |
| | Chr4: 16477393-16479266 REVERSE LENGTH = 377 |
| 4869 | AT4G34540.1 Symbols: no symbol available: no full name available. Chr4: 16500506-16501932 |
| | FORWARD LENGTH = 306 |
| 4870 | AT4G34560.1 Symbols: no symbol available: no full name available. Chr4: 16507923-16508588 |
| | FORWARD LENGTH = 221 |
| 4871 | AT4G34590.1 Symbols: BZIP11, ATB2, GBF6, AtbZIP11: Arabidopsis thaliana basic leucine-zipper 11, G- |
| | box binding factor 6. Chr4: 16522449-16522928 FORWARD LENGTH = 159 |
| 4872 | AT4G34710.1 Symbols: SPE2, ADC2, ATADC2: arginine decarboxylase 2. Chr4: 16560315-16562450 |
| | REVERSE LENGTH = 711 |
| 4873 | AT4G34881.1 Symbols: no symbol available: no full name available. Chr4: 16617892-16618071 |
| | FORWARD LENGTH = 59 |
| 4874 | AT4G35020.1 Symbols: ARAC3, ATROP6, RHO1PS, RAC3, ROP6: RHO-RELATED PROTEIN FROM |
| | PLANTS 6, RAC-like 3. Chr4: 16673176-16674540 FORWARD LENGTH = 198 |
| 4875 | AT4G35040.1 Symbols: bZIP19.: Chr4: 16680821-16681805 FORWARD LENGTH = 261 |
| 4876 | AT4G35390.1 Symbols: AGF1, AHL25: AT-hook motif nuclear localized protein 25, AT-hook protein of |
| | GA feedback 1. Chr4: 16829603-16830502 FORWARD LENGTH = 299 |
| 4877 | AT4G35470.1 Symbols: PIRL4: plant intracellular ras group-related LRR 4. Chr4: 16846531-16848448 |
| | FORWARD LENGTH = 549 |
| 4878 | AT4G35860.1 Symbols: ATRABB1B, ATGB2, ATRAB2C, GB2: GTP-binding 2. Chr4: 16987118- |
| | 16988839 REVERSE LENGTH = 211 |
| 4879 | AT4G35920.1 Symbols: MCAL: mid1-complementing activity 1. Chr4: 17012106-17014192 REVERSE |
| | LENGTH = 421 |
| 4880 | AT4G35940.1 Symbols: no symbol available: no full name available. Chr4: 17021672-17023593 |
| | FORWARD LENGTH = 451 |
| 4881 | AT4G36050.2 Symbols: no symbol available: no full name available. Chr4: 17052323-17055044 |
| | REVERSE LENGTH = 610 |
| 4882 | AT4G36090.3 Symbols: no symbol available: no full name available. Chr4: 17078376-17080670 |
| | REVERSE LENGTH = 520 |
| 4883 | AT4G36620.1 Symbols: GATA19, HANL2: GATA transcription factor 19, hanaba taranu like 2. |
| | Chr4: 17268906-17269662 REVERSE LENGTH = 211 |
| 4884 | AT4G36840.1 Symbols: no symbol available: no full name available. Chr4: 17352166-17352882 |
| | FORWARD LENGTH = 238 |
| 4885 | AT4G36960.1 Symbols: no symbol available: no full name available. Chr4: 17427349-17429589 |
| | FORWARD LENGTH = 379 |
| 4886 | AT4G37080.2 Symbols: no symbol available: no full name available. Chr4: 17473719-17476716 |
| | FORWARD LENGTH = 610 |
| 4887 | AT4G37590.1 Symbols: MEL1, NPY5: MAB4/ENP/NPY1-LIKE 1, NAKED PINS IN YUC MUTANTS |
| | 5. Chr4: 17663080-17665299 REVERSE LENGTH = 580 |
| 4888 | AT4G37608.1 Symbols: no symbol available: no full name available. Chr4: 17669825-17670429 |
| | FORWARD LENGTH = 87 |
| 4889 | AT4G37690.1 Symbols: no symbol available: no full name available. Chr4: 17708303-17709601 |
| | FORWARD LENGTH = 432 |
| 4890 | AT4G37750.1 Symbols: ANT, CKC1, DRG, CKC, AtANT: COMPLEMENTING A PROTEIN KINASE C |
| | MUTANT 1, AINTEGUMENTA, DRAGON. Chr4: 17739782-17742189 FORWARD LENGTH = 555 |
| 4891 | AT4G38260.1 Symbols: no symbol available: no full name available. Chr4: 17937077-17938210 |
| | REVERSE LENGTH = 275 |
| 4892 | AT4G38330.1 Symbols: no symbol available: no full name available. Chr4: 17952294-17952540 |
| | FORWARD LENGTH = 47 |
| 4893 | AT4G38520.1 Symbols: APD6: Arabidopsis Pp2c clade D 6. Chr4: 18015999-18017514 REVERSE |
| | LENGTH = 400 |
| 4894 | AT4G38950.1 Symbols: no symbol available: no full name available. Chr4: 18154606-18158461 |
| | REVERSE LENGTH = 836 |
| 4895 | AT4G38960.3 Symbols: BBX19: B-box domain protein 19. Chr4: 18161576-18163045 FORWARD |
| | LENGTH = 226 |
| 4896 | AT4G39050.1 Symbols: Kin7.4: kinesin 7.4. Chr4: 18193462-18200148 FORWARD LENGTH = 1055 |
| 4897 | AT4G39180.1 Symbols: ATSEC14, SEC14: SECRETION 14, ARABIDOPSIS THALIANA SECRETION |
| | 14. Chr4: 18244006-18246673 REVERSE LENGTH = 554 |
| 4898 | AT4G39190.1 Symbols: no symbol available: no full name available. Chr4: 18252032-18252865 |
| | REVERSE LENGTH = 277 |
| 4899 | AT4G39270.1 Symbols: no symbol available: no full name available. Chr4: 18276874-18279710 |
| | FORWARD LENGTH = 864 |
| 4900 | AT4G39390.1 Symbols: NST-K1, ATNST-KT1, URGT4: UDP-Rha/UDP-Gal transporter 4, nucleotide |
| | sugar transporter-KT 1, A. THALIANA NUCLEOTIDE SUGAR TRANSPORTER-KT 1. |
| | Chr4: 18316278-18317854 FORWARD LENGTH = 337 |
| 4901 | AT4G39420.2 Symbols: no symbol available: no full name available. Chr4: 18339731-18354701 |
| | FORWARD LENGTH = 3184 |
| 4902 | AT4G39680.1 Symbols: no symbol available: no full name available. Chr4: 18414604-18416938 |
| | REVERSE LENGTH = 633 |
| 4903 | AT4G40030.2 Symbols: H3.3, HTR4: histone 3.3. Chr4: 18555840-18556827 REVERSE LENGTH = 164 |
| 4904 | AT4G40040.1 Symbols: H3.3, HTR5: histone 3.3. Chr4: 18557539-18558236 REVERSE LENGTH = 136 |
| 4905 | AT5G01100.1 Symbols: FRB1: FRIABLE 1. Chr5: 34872-37756 REVERSE LENGTH = 631 |
| 4906 | AT5G01300.1 Symbols: no symbol available: no full name available. Chr5: 121643-122448 REVERSE |
| | LENGTH = 162 |
| 4907 | AT5G01380.1 Symbols: no symbol available: no full name available. Chr5: 155784-157451 REVERSE |
| | LENGTH = 323 |
| 4908 | AT5G01520.1 Symbols: AtAIRP2, AIRP2: ABA Insensitive RING Protein 2. Chr5: 206797-208399 |
| | FORWARD LENGTH = 242 |
| 4909 | AT5G01710.1 Symbols: no symbol available: no full name available. Chr5: 263709-265250 REVERSE |
| | LENGTH = 513 |
| 4910 | AT5G01810.1 Symbols: ATPK10, SNRK3.1, CIPK15, SIP2, PKS3: SNF1-RELATED PROTEIN KINASE |
| | 3.1, PROTEIN KINASE 10, SOS3-INTERACTING PROTEIN 2, CBL-interacting protein kinase 15. |
| | Chr5: 310460-311725 FORWARD LENGTH = 421 |
| 4911 | AT5G01830.1 Symbols: no symbol available: no full name available. Chr5: 320983-323007 FORWARD |
| | LENGTH = 674 |
| 4912 | AT5G02060.1 Symbols: CASPL5B1: CASP-like protein 5B1. Chr5: 404908-405774 FORWARD |
| | LENGTH = 152 |
| 4913 | AT5G02480.1 Symbols: no symbol available: no full name available. Chr5: 548152-549678 FORWARD |
| | LENGTH = 508 |
| 4914 | AT5G02550.1 Symbols: no symbol available: no full name available. Chr5: 573247-573477 REVERSE |
| | LENGTH = 76 |
| 4915 | AT5G02650.1 Symbols: no symbol available: no full name available. Chr5: 598276-599786 FORWARD |
| | LENGTH = 226 |
| 4916 | AT5G02680.1 Symbols: no symbol available: no full name available. Chr5: 606065-607884 FORWARD |
| | LENGTH = 133 |
| 4917 | AT5G03190.2 Symbols: CPUORF47: conserved peptide upstream open reading frame 47. Chr5: 758539- |
| | 760148 FORWARD LENGTH = 475 |
| 4918 | AT5G03240.1 Symbols: UBQ3: polyubiquitin 3. Chr5: 771976-772896 REVERSE LENGTH = 306 |
| 4919 | AT5G03280.1 Symbols: EIN2, CKR1, ATEIN2, ERA3, PIR2, ORE2, ORE3: ENHANCED RESPONSE TO |
| | ABA3, CYTOKININ RESISTANT 1, ORESARA 2, ETHYLENE INSENSITIVE 2, ORESARA 3. |
| | Chr5: 788589-793066 FORWARD LENGTH = 1294 |
| 4920 | AT5G03330.1 Symbols: no symbol available: no full name available. Chr5: 807728-809608 FORWARD |
| | LENGTH = 356 |
| 4921 | AT5G03500.1 Symbols: MED7B: MED7B. Chr5: 876673-877660 REVERSE LENGTH = 168 |
| 4922 | AT5G03540.3 Symbols: ATEXO70A1, EXO70A1: exocyst subunit exo70 family protein A1. |
| | Chr5: 889606-893916 FORWARD LENGTH = 664 |
| 4923 | AT5G03795.1 Symbols: no symbol available: no full name available. Chr5: 1007554-1010373 |
| | REVERSE LENGTH = 518 |
| 4924 | AT5G03990.1 Symbols: no symbol available: no full name available. Chr5: 1075957-1077358 |
| | FORWARD LENGTH = 302 |
| 4925 | AT5G04220.2 Symbols: NTMC2TYPE1.3, NTMC2T1.3, ATSYTC, SYT3, SYTC: synaptotagmin 3. |
| | Chr5: 1155985-1158620 REVERSE LENGTH = 540 |
| 4926 | AT5G04320.2 Symbols: SGO2, AtSGO2: SHUGOSHIN 2. Chr5: 1209613-1212376 FORWARD |
| | LENGTH = 470 |
| 4927 | AT5G04480.1 Symbols: BUP: Bursting pollen. Chr5: 1271886-1277793 REVERSE LENGTH = 1050 |
| 4928 | AT5G04920.1 Symbols: no symbol available: no full name available. Chr5: 1439172-1441545 |
| | REVERSE LENGTH = 440 |
| 4929 | AT5G04940.1 Symbols: SUVH1: SU(VAR)3-9 homolog 1. Chr5: 1454616-1456628 REVERSE |
| | LENGTH = 670 |
| 4930 | AT5G05210.1 Symbols: no symbol available: no full name available. Chr5: 1548198-1549534 |
| | FORWARD LENGTH = 386 |
| 4931 | AT5G05350.1 Symbols: no symbol available: no full name available. Chr5: 1585757-1587337 |
| | FORWARD LENGTH = 526 |
| 4932 | AT5G05930.1 Symbols: GC1, ATGC1: ARABIDOPSIS GUANYLYL CYCLASE 1, guanylyl cyclase 1. |
| | Chr5: 1780270-1782659 REVERSE LENGTH = 274 |
| 4933 | AT5G06100.2 Symbols: ATMYB33, MYB33: myb domain protein 33. Chr5: 1838555-1840386 |
| | FORWARD LENGTH = 520 |
| 4934 | AT5G06130.2 Symbols: AtOR-like: Arabidopsis thaliana orange-like. Chr5: 1853754-1855763 |
| | REVERSE LENGTH = 315 |
| 4935 | AT5G06265.1 Symbols: no symbol available: no full name available. Chr5: 1906596-1907382 |
| | FORWARD LENGTH = 106 |
| 4936 | AT5G06420.1 Symbols: no symbol available: no full name available. Chr5: 1961521-1962870 |
| | FORWARD LENGTH = 378 |
| 4937 | AT5G06750.1 Symbols: APD8: Arabidopsis Pp2c clade D 8. Chr5: 2086403-2088245 REVERSE |
| | LENGTH = 393 |
| 4938 | AT5G06839.3 Symbols: bZIP65, TGA10: TGACG (TGA) motif-binding protein 10. Chr5: 2120911- |
| | 2126217 FORWARD LENGTH = 460 |
| 4939 | AT5G07370.2 Symbols: IPK2a, IMPK, ATIPK2A: inositol polyphosphate kinase 2 alpha, inositol multi- |
| | phosphate kinase, INOSITOL POLYPHOSPHATE KINASE 2 ALPHA. Chr5: 2331251-2332111 |
| | FORWARD LENGTH = 286 |
| 4940 | AT5G07790.1 Symbols: no symbol available: no full name available. Chr5: 2484073-2486107 |
| | FORWARD LENGTH = 616 |
| 4941 | AT5G07840.1 Symbols: PIA1: phytochrome interacting ankyrin-repeat protein 1. Chr5: 2506764- |
| | 2507291 REVERSE LENGTH = 175 |
| 4942 | AT5G07890.1 Symbols: no symbol available: no full name available. Chr5: 2517718-2519493 |
| | REVERSE LENGTH = 409 |
| 4943 | AT5G07920.1 Symbols: ATDGK1, DGK1: diacylglycerol kinase1, DIACYLGLYCEROL KINASE 1. |
| | Chr5: 2525197-2528396 REVERSE LENGTH = 728 |
| 4944 | AT5G08100.1 Symbols: ASPGA1: asparaginase A1. Chr5: 2593242-2594586 REVERSE LENGTH = 315 |
| 4945 | AT5G08130.5 Symbols: BIM1.: Chr5: 2606655-2609571 REVERSE LENGTH = 532 |
| 4946 | AT5G08560.1 Symbols: WDR26, ATWDR26: WD-40 repeat 26. Chr5: 2771104-2773827 REVERSE |
| | LENGTH = 589 |
| 4947 | AT5G09350.1 Symbols: PI-4KBETA2, PI4KBETA2: phosphatidylinositol 4-OH kinase |
| | beta2, PHOSPHATIDYLINOSITOL 4-OH KINASE BETA2. Chr5: 2899365-2905610 REVERSE |
| | LENGTH = 1116 |
| 4948 | AT5G09460.1 Symbols: no symbol available: no full name available. Chr5: 2944437-2945417 |
| | REVERSE LENGTH = 326 |
| 4949 | AT5G09600.1 Symbols: SDH3-1: succinate dehydrogenase 3-1. Chr5: 2979220-2980527 FORWARD |
| | LENGTH = 213 |
| 4950 | AT5G09630.1 Symbols: no symbol available: no full name available. Chr5: 2986016-2987176 |
| | REVERSE LENGTH = 386 |
| 4951 | AT5G09670.2 Symbols: no symbol available: no full name available. Chr5: 2996109-2997749 |
| | REVERSE LENGTH = 546 |
| 4952 | AT5G09680.1 Symbols: RLF: reduced lateral root formation. Chr5: 2999362-3000185 REVERSE |
| | LENGTH = 211 |
| 4953 | AT5G09850.1 Symbols: no symbol available: no full name available. Chr5: 3063488-3065221 |
| | REVERSE LENGTH = 353 |
| 4954 | AT5G10030.1 Symbols: TGA4, OBF4: TGACG motif-binding factor 4, OCS ELEMENT BINDING |
| | FACTOR 4. Chr5: 3137648-3139295 REVERSE LENGTH = 364 |
| 4955 | AT5G10510.3 Symbols: AIL6, PLT3: PLETHORA 3, AINTEGUMENTA-like 6. Chr5: 3315991-3320008 |
| | FORWARD LENGTH = 604 |
| 4956 | AT5G11150.1 Symbols: VAMP713, ATVAMP713: vesicle-associated membrane protein 713. |
| | Chr5: 3546625-3547844 REVERSE LENGTH = 221 |
| 4957 | AT5G11730.1 Symbols: no symbol available: no full name available. Chr5: 3780963-3782473 |
| | FORWARD LENGTH = 386 |
| 4958 | AT5G11860.1 Symbols: SSP5: SCP1-like small phosphatase 5. Chr5: 3822024-3823395 REVERSE |
| | LENGTH = 305 |
| 4959 | AT5G12440.1 Symbols: no symbol available: no full name available. Chr5: 4035891-4038604 |
| | REVERSE LENGTH = 650 |
| 4960 | AT5G12480.1 Symbols: CPK7: calmodulin-domain protein kinase 7. Chr5: 4047817-4050035 REVERSE |
| | LENGTH = 535 |
| 4961 | AT5G12840.1 Symbols: NF-YA1, EMB2220, HAP2A, NFYA1, AtNFYA1, ATHAP2A: “nuclear factor Y, |
| | subunit A1”, nuclear factor Y subunit A1, EMBRYO DEFECTIVE 2220. Chr5: 4051147-4052961 |
| | REVERSE LENGTH = 272 |
| 4962 | AT5G13290.2 Symbols: SOL2, CRN: SUPPRESSOR OF LLP1 2, CORYNE. Chr5: 4252924-4254215 |
| | REVERSE LENGTH = 401 |
| 4963 | AT5G13660.2 Symbols: no symbol available: no full name available. Chr5: 4405094-4406983 |
| | FORWARD LENGTH = 537 |
| 4964 | AT5G13840.1 Symbols: CCS52B, FZR3: FIZZY-related 3, cell cycle switch protein 52 B. Chr5: 4468677- |
| | 4470706 REVERSE LENGTH = 481 |
| 4965 | AT5G14310.1 Symbols: AtCXE16, CXE16: carboxyesterase 16. Chr5: 4615592-4617422 FORWARD |
| | LENGTH = 446 |
| 4966 | AT5G14720.1 Symbols: no symbol available: no full name available. Chr5: 4748212-4752642 |
| | REVERSE LENGTH = 674 |
| 4967 | AT5G14740.1 Symbols: BETA CA2, CA2, CA18: BETA CARBONIC ANHYDRASE 2, CARBONIC |
| | ANHYDRASE 18, carbonic anhydrase 2. Chr5: 4758257-4762382 FORWARD LENGTH = 331 |
| 4968 | AT5G15020.1 Symbols: SNL2: SIN3-like 2. Chr5: 4859408-4865569 REVERSE LENGTH = 1367 |
| 4969 | AT5G15260.1 Symbols: no symbol available: no full name available. Chr5: 4953646-4954350 |
| | REVERSE LENGTH = 234 |
| 4970 | AT5G15320.1 Symbols: no symbol available: no full name available. Chr5: 4977647-4978658 |
| | FORWARD LENGTH = 53 |
| 4971 | AT5G15400.1 Symbols: MUSE3: Mutant, snc1 -enhancing 3. Chr5: 4997764-5002907 REVERSE |
| | LENGTH = 1038 |
| 4972 | AT5G15580.1 Symbols: TRM2, LNG1: LONGIFOLIAI, TONI Recruiting Motif 2. Chr5: 5068495- |
| | 5071930 REVERSE LENGTH = 927 |
| 4973 | AT5G15790.1 Symbols: no symbol available: no full name available. Chr5: 5149800-5150964 |
| | FORWARD LENGTH = 232 |
| 4974 | AT5G15950.1 Symbols: no symbol available: no full name available. Chr5: 5206706-5207794 |
| | FORWARD LENGTH = 362 |
| 4975 | AT5G16140.1 Symbols: no symbol available: no full name available. Chr5: 5270308-5271517 |
| | REVERSE LENGTH = 240 |
| 4976 | AT5G16280.1 Symbols: no symbol available: no full name available. Chr5: 5323377-5331345 |
| | REVERSE LENGTH = 1272 |
| 4977 | AT5G16510.1 Symbols: RGP5: reversibly glycosylated polypeptide 5, reversibly glycosylated protein 5. |
| | Chr5: 5393296-5394342 FORWARD LENGTH = 348 |
| 4978 | AT5G16610.2 Symbols: no symbol available: no full name available. Chr5: 5445068-5447945 |
| | REVERSE LENGTH = 673 |
| 4979 | AT5G17460.2 Symbols: no symbol available: no full name available. Chr5: 5757582-5758867 |
| | REVERSE LENGTH = 309 |
| 4980 | AT5G18590.1 Symbols: no symbol available: no full name available. Chr5: 6178516-6182186 |
| | REVERSE LENGTH = 708 |
| 4981 | AT5G18690.1 Symbols: ATAGP25, AGP25: arabinogalactan protein 25, ARABIDOPSIS THALIANA |
| | ARABINOGALACTAN PROTEINS 25. Chr5: 6233776-6234126 REVERSE LENGTH = 116 |
| 4982 | AT5G18860.1 Symbols: NSH3: nucleoside hydrolase 3. Chr5: 6291023-6295581 FORWARD |
| | LENGTH = 890 |
| 4983 | AT5G19420.2 Symbols: no symbol available: no full name available. Chr5: 6547945-6552981 |
| | REVERSE LENGTH = 1139 |
| 4984 | AT5G19430.1 Symbols: C3HC4.: Chr5: 6553998-6555616 FORWARD LENGTH = 255 |
| 4985 | AT5G19670.1 Symbols: no symbol available: no full name available. Chr5: 6647025-6649358 |
| | FORWARD LENGTH = 610 |
| 4986 | AT5G20250.4 Symbols: RS6, DIN10: raffinose synthase 6, DARK INDUCIBLE 10. Chr5: 6833730- |
| | 6836635 FORWARD LENGTH = 844 |
| 4987 | AT5G20450.1 Symbols: no symbol available: no full name available. Chr5: 6910968-6913132 |
| | REVERSE LENGTH = 341 |
| 4988 | AT5G20680.1 Symbols: TBL16: TRICHOME BIREFRINGENCE-LIKE 16. Chr5: 6998946-7001596 |
| | FORWARD LENGTH = 551 |
| 4989 | AT5G20930.1 Symbols: TSL: TOUSLED. Chr5: 7098213-7102970 FORWARD LENGTH = 688 |
| 4990 | AT5G21910.1 Symbols: no symbol available: no full name available. Chr5: 7240789-7241127 |
| | FORWARD LENGTH = 112 |
| 4991 | AT5G22130.2 Symbols: PNT1: PEANUT 1. Chr5: 7337486-7339831 REVERSE LENGTH = 470 |
| 4992 | AT5G22940.1 Symbols: F8H: FRA8 homolog. Chr5: 7677197-7678892 FORWARD LENGTH = 469 |
| 4993 | AT5G23450.3 Symbols: ATLCBK1, LCBK1: long-chain base (LCB) kinase 1. Chr5: 7905041-7908960 |
| | REVERSE LENGTH = 778 |
| 4994 | AT5G23610.2 Symbols: no symbol available: no full name available. Chr5: 7957641-7959968 |
| | REVERSE LENGTH = 500 |
| 4995 | AT5G24105.1 Symbols: AGP41: arabinogalactan protein 41. Chr5: 8152097-8152656 FORWARD |
| | LENGTH = 63 |
| 4996 | AT5G24360.2 Symbols: AtIRE1b, ATIRE1-1, IRE1-1, IRE1B: ARABIDOPSIS THALIANA INOSITOL |
| | REQUIRING 1-1, inositol requiring 1-1. Chr5: 8316718-8319827 FORWARD LENGTH = 887 |
| 4997 | AT5G24760.1 Symbols: no symbol available: no full name available. Chr5: 8495035-8497245 |
| | REVERSE LENGTH = 381 |
| 4998 | AT5G24970.2 Symbols: no symbol available: no full name available. Chr5: 8604358-8608656 |
| | REVERSE LENGTH = 761 |
| 4999 | AT5G25110.1 Symbols: CIPK25, SnRK3.25: CBL-interacting protein kinase 25, SNF1-RELATED |
| | PROTEIN KINASE 3.25. Chr5: 8657740-8659206 REVERSE LENGTH = 488 |
| 5000 | AT5G25560.2 Symbols: no symbol available: no full name available. Chr5: 8899486-8901391 |
| | FORWARD LENGTH = 308 |
| 5001 | AT5G25620.2 Symbols: AtYUC6, YUC6: YUCCA6. Chr5: 8935312-8938224 REVERSE LENGTH = 434 |
| 5002 | AT5G25760.1 Symbols: PEX4, UBC21: peroxin4, ubiquitin-conjugating enzyme 21. Chr5: 8967983- |
| | 8969173 FORWARD LENGTH = 157 |
| 5003 | AT5G26622.1 Symbols: no symbol available: no full name available. Chr5: 9356004-9356891 |
| | REVERSE LENGTH = 295 |
| 5004 | AT5G26740.1 Symbols: no symbol available: no full name available. Chr5: 9292436-9294407 |
| | FORWARD LENGTH = 422 |
| 5005 | AT5G26910.1 Symbols: TRM8: TON1 Recruiting Motif 8. Chr5: 9466169-9469523 REVERSE |
| | LENGTH = 853 |
| 5006 | AT5G26940.1 Symbols: DPD1: defective in pollen organelle DNA degradationl. Chr5: 9481429- |
| | 9482647 FORWARD LENGTH = 316 |
| 5007 | AT5G27950.1 Symbols: no symbol available: no full name available. Chr5: 9984774-9987493 |
| | FORWARD LENGTH = 625 |
| 5008 | AT5G28850.2 Symbols: no symbol available: no full name available. Chr5: 10877360-10881278 |
| | REVERSE LENGTH = 536 |
| 5009 | AT5G33210.1 Symbols: SRS8: SHI-related sequence 8. Chr5: 12465057-12465713 REVERSE |
| | LENGTH = 173 |
| 5010 | AT5G35980.1 Symbols: YAK1: yeast YAK1-related gene 1. Chr5: 14128551-14135984 FORWARD |
| | LENGTH = 956 |
| 5011 | AT5G35995.1 Symbols: no symbol available: no full name available. Chr5: 14136707-14138230 |
| | REVERSE LENGTH = 455 |
| 5012 | AT5G37370.1 Symbols: ATSRL1.: Chr5: 14812845-14815988 REVERSE LENGTH = 393 |
| 5013 | AT5G37380.1 Symbols: no symbol available: no full name available. Chr5: 14817035-14818330 |
| | REVERSE LENGTH = 431 |
| 5014 | AT5G38590.2 Symbols: no symbol available: no full name available. Chr5: 15452066-15453455 |
| | REVERSE LENGTH = 410 |
| 5015 | AT5G38970.1 Symbols: CYP85A1, ATBR6OX, BR6OX, BR6OX1: brassinosteroid-6-oxidase |
| | 1, BRASSINOSTEROID-6-OXIDASE. Chr5: 15594935-15597774 REVERSE LENGTH = 465 |
| 5016 | AT5G39360.1 Symbols: EDL2: EID1-like 2. Chr5: 15753313-15754062 REVERSE LENGTH = 249 |
| 5017 | AT5G39450.1 Symbols: no symbol available: no full name available. Chr5: 15786045-15787877 |
| | FORWARD LENGTH = 579 |
| 5018 | AT5G40370.2 Symbols: AtGRXC2, GRXC2: glutaredoxin C2. Chr5: 16147826-16148900 REVERSE |
| | LENGTH = 136 |
| 5019 | AT5G40760.1 Symbols: G6PD6: glucose-6-phosphate dehydrogenase 6. Chr5: 16311284-16314556 |
| | FORWARD LENGTH = 515 |
| 5020 | AT5G40810.1 Symbols: no symbol available: no full name available. Chr5: 16340200-16342327 |
| | FORWARD LENGTH = 307 |
| 5021 | AT5G41110.1 Symbols: no symbol available: no full name available. Chr5: 16452598-16454989 |
| | FORWARD LENGTH = 621 |
| 5022 | AT5G41410.1 Symbols: BEL1: BELL 1. Chr5: 16580424-16583770 FORWARD LENGTH = 611 |
| 5023 | AT5G41610.1 Symbols: CHX18, ATCHX18: ARABIDOPSIS THALIANA CATION/H+ EXCHANGER |
| | 18, cation/H+ exchanger 18. Chr5: 16638554-16641146 REVERSE LENGTH = 810 |
| 5024 | AT5G42560.1 Symbols: no symbol available: no full name available. Chr5: 17015573-17016969 |
| | FORWARD LENGTH = 296 |
| 5025 | AT5G43320.1 Symbols: ckl8, CK1.8: casein kinase 1-like 8. Chr5: 17386043-17388941 REVERSE |
| | LENGTH = 480 |
| 5026 | AT5G43810.1 Symbols: PNH, AGO10, ZLL: PINHEAD, ZWILLE, ARGONAUTE 10. Chr5: 17611939- |
| | 17616562 FORWARD LENGTH = 988 |
| 5027 | AT5G44040.1 Symbols: no symbol available: no full name available. Chr5: 17719896-17721348 |
| | REVERSE LENGTH = 416 |
| 5028 | AT5G44568.1 Symbols: no symbol available: no full name available. Chr5: 17961379-17962146 |
| | REVERSE LENGTH = 75 |
| 5029 | AT5G44800.1 Symbols: CHR4, PKR1: chromatin remodeling 4, PICKLE RELATED 1. Chr5: 18083659- |
| | 18092162 REVERSE LENGTH = 2223 |
| 5030 | AT5G44980.1 Symbols: no symbol available: no full name available. Chr5: 18160600-18162058 |
| | FORWARD LENGTH = 435 |
| 5031 | AT5G45100.1 Symbols: BRG1: BOI-related gene 1. Chr5: 18218769-18219741 FORWARD |
| | LENGTH = 294 |
| 5032 | AT5G45430.1 Symbols: no symbol available: no full name available. Chr5: 18409200-18411711 |
| | FORWARD LENGTH = 499 |
| 5033 | AT5G45470.1 Symbols: no symbol available: no full name available. Chr5: 18422164-18424764 |
| | REVERSE LENGTH = 866 |
| 5034 | AT5G45710.1 Symbols: AT-HSFA4C, RHA1, HSFA4C: HEAT SHOCK TRANSCRIPTION FACTOR |
| | A4C, ROOT HANDEDNESS 1. Chr5: 18541576-18542706 FORWARD LENGTH = 345 |
| 5035 | AT5G45820.1 Symbols: PKS18, SnRK3.6, CIPK20: PROTEIN KINASE 18, SNF1-RELATED PROTEIN |
| | KINASE 3.6, CBL-interacting protein kinase 20. Chr5: 18587081-18588400 REVERSE LENGTH = 439 |
| 5036 | AT5G47050.1 Symbols: no symbol available: no full name available. Chr5: 19106612-19107860 |
| | FORWARD LENGTH = 300 |
| 5037 | AT5G47070.1 Symbols: no symbol available: no full name available. Chr5: 19118683-19120528 |
| | REVERSE LENGTH = 410 |
| 5038 | AT5G47100.1 Symbols: CBL9, ATCBL9: calcineurin B-like protein 9. Chr5: 19129896-19131727 |
| | REVERSE LENGTH = 213 |
| 5039 | AT5G47220.1 Symbols: ATERF-2, ATERF2, ERF2: ETHYLENE RESPONSIVE ELEMENT BINDING |
| | FACTOR 2, ethylene responsive element binding factor 2, ETHYLENE RESPONSE FACTOR- 2. |
| | Chr5: 19172023-19172754 REVERSE LENGTH = 243 |
| 5040 | AT5G47250.1 Symbols: no symbol available: no full name available. Chr5: 19186045-19188576 |
| | REVERSE LENGTH = 843 |
| 5041 | AT5G47430.1 Symbols: no symbol available: no full name available. Chr5: 19235819-19240596 |
| | REVERSE LENGTH = 892 |
| 5042 | AT5G47520.1 Symbols: RABA5a, AtRABA5a: RAB GTPase homolog A5A. Chr5: 19277596-19278366 |
| | REVERSE LENGTH = 221 |
| 5043 | AT5G47710.1 Symbols: no symbol available: no full name available. Chr5: 19330470-19331178 |
| | FORWARD LENGTH = 166 |
| 5044 | AT5G47720.2 Symbols: AACT1: acetoacetyl-CoA thiolase 1. Chr5: 19331762-19334145 FORWARD |
| | LENGTH = 415 |
| 5045 | AT5G47730.1 Symbols: no symbol available: no full name available. Chr5: 19334592-19336618 |
| | REVERSE LENGTH = 341 |
| 5046 | AT5G47970.1 Symbols: no symbol available: no full name available. Chr5: 19423420-19425585 |
| | FORWARD LENGTH = 387 |
| 5047 | AT5G48220.1 Symbols: no symbol available: no full name available. Chr5: 19549930-19552046 |
| | FORWARD LENGTH = 379 |
| 5048 | AT5G48230.2 Symbols: EMB1276, ACAT2, AACT2: acetoacetyl-CoA thiolasc 2, EMBRYO |
| | DEFECTIVE 1276, Acetoacetyl-CoA thiolase 2. Chr5: 19552570-19555122 REVERSE LENGTH = 403 |
| 5049 | AT5G48580.1 Symbols: FKBP15-2: FK506- and rapamycin-binding protein 15 kD-2. Chr5: 19696156- |
| | 19697304 REVERSE LENGTH = 163 |
| 5050 | AT5G48610.1 Symbols: no symbol available: no full name available. Chr5: 19712614-19714861 |
| | FORWARD LENGTH = 354 |
| 5051 | AT5G48655.2 Symbols: no symbol available: no full name available. Chr5: 19731575-19732486 |
| | REVERSE LENGTH = 203 |
| 5052 | AT5G48660.1 Symbols: no symbol available: no full name available. Chr5: 19736986-19738327 |
| | FORWARD LENGTH = 219 |
| 5053 | AT5G49470.2 Symbols: RAF10.: Chr5: 20063616-20068311 FORWARD LENGTH = 831 |
| 5054 | AT5G49610.1 Symbols: no symbol available: no full name available. Chr5: 20131448-20132527 |
| | REVERSE LENGTH = 359 |
| 5055 | AT5G50010.1 Symbols: no symbol available: no full name available. Chr5: 20349556-20350491 |
| | FORWARD LENGTH = 311 |
| 5056 | AT5G50180.1 Symbols: no symbol available: no full name available. Chr5: 20431116-20432883 |
| | FORWARD LENGTH = 346 |
| 5057 | AT5G50400.1 Symbols: PAP27, ATPAP27: ARABIDOPSIS THALIANA PURPLE ACID |
| | PHOSPHATASE 27, purple acid phosphatase 27. Chr5: 20523575-20526231 REVERSE LENGTH = 611 |
| 5058 | AT5G50430.1 Symbols: UBC33: ubiquitin-conjugating enzyme 33. Chr5: 20534366-20536307 |
| | REVERSE LENGTH = 243 |
| 5059 | AT5G50565.1 Symbols: no symbol available: no full name available. Chr5: 20580894-20581826 |
| | FORWARD LENGTH = 75 |
| 5060 | AT5G51060.1 Symbols: RHD2, RBOHC, ATRBOHC: ROOT HAIR DEFECTIVE 2, RESPIRATORY |
| | BURST OXIDASE HOMOLOG C, A. THALIANA RESPIRATORY BURST OXIDASE HOMOLOG C. |
| | Chr5: 20757484-20762175 REVERSE LENGTH = 905 |
| 5061 | AT5G52030.2 Symbols: no symbol available: no full name available. Chr5: 21128291-21130034 |
| | FORWARD LENGTH = 402 |
| 5062 | AT5G52040.2 Symbols: ATRSP41, RS41, At-RS41: arginine/serine-rich splicing factor 41. |
| | Chr5: 21131081-21133318 FORWARD LENGTH = 357 |
| 5063 | AT5G52530.1 Symbols: no symbol available: no full name available. Chr5: 21318215-21320882 |
| | FORWARD LENGTH = 828 |
| 5064 | AT5G52550.1 Symbols: no symbol available: no full name available. Chr5: 21327914-21328996 |
| | REVERSE LENGTH = 360 |
| 5065 | AT5G52860.1 Symbols: ABCG8: ATP-binding cassette G8. Chr5: 21419776-21421545 REVERSE |
| | LENGTH = 589 |
| 5066 | AT5G52890.2 Symbols: no symbol available: no full name available. Chr5: 21445040-21446197 |
| | REVERSE LENGTH = 385 |
| 5067 | AT5G53180.1 Symbols: PTB2, ATPTB2: polypyrimidine tract-binding protein 2, POLYPYRIMIDINE |
| | TRACT-BINDING PROTEIN 2. Chr5: 21568393-21571537 REVERSE LENGTH = 429 |
| 5068 | AT5G53250.1 Symbols: AGP22, ATAGP22: ARABINOGALACTAN PROTEIN 22, arabinogalactan |
| | protein 22. Chr5: 21603715-21604007 FORWARD LENGTH = 63 |
| 5069 | AT5G53550.1 Symbols: YSL3, ATYSL3: YELLOW STRIPE like 3, YELLOW STRIPE LIKE 3. |
| | Chr5: 21756081-21758776 FORWARD LENGTH = 675 |
| 5070 | AT5G53590.1 Symbols: SAUR30: SMALL AUXIN UPREGULATED RNA 30. Chr5: 21772107- |
| | 21772535 FORWARD LENGTH = 142 |
| 5071 | AT5G53620.1 Symbols: no symbol available: no full name available. Chr5: 21781217-21785061 |
| | FORWARD LENGTH = 682 |
| 5072 | AT5G53760.1 Symbols: MLO11, ATMLO11: MILDEW RESISTANCE LOCUS O 11. Chr5: 21823055- |
| | 21826289 FORWARD LENGTH = 573 |
| 5073 | AT5G53880.1 Symbols: no symbol available: no full name available. Chr5: 21872995-21873195 |
| | REVERSE LENGTH = 66 |
| 5074 | AT5G54130.2 Symbols: no symbol available: no full name available. Chr5: 21962900-21965279 |
| | FORWARD LENGTH = 436 |
| 5075 | AT5G54650.1 Symbols: Fh5, ATFH5: FORMIN HOMOLOGY 5, formin homology5. Chr5: 22197856- |
| | 22201649 REVERSE LENGTH = 900 |
| 5076 | AT5G54670.1 Symbols: KATC, ATK3: kinesin 3, KINESIN-LIKE PROTEIN IN ARABIDOPSIS |
| | THALIANA C. Chr5: 22209912-22213843 FORWARD LENGTH = 754 |
| 5077 | AT5G54730.1 Symbols: ATATG18F, G18F, ATG18F: ARABIDOPSIS THALIANA HOMOLOG OF |
| | YEAST AUTOPHAGY 18 (ATG18) F, homolog of yeast autophagy 18 (ATG18) F. Chr5: 22233977- |
| | 22236804 REVERSE LENGTH = 763 |
| 5078 | AT5G54760.1 Symbols: no symbol available: no full name available. Chr5: 22244732-22245517 |
| | FORWARD LENGTH = 113 |
| 5079 | AT5G55120.1 Symbols: VTC5: VITAMIN C DEFECTIVE 5. Chr5: 22369515-22371709 FORWARD |
| | LENGTH = 431 |
| 5080 | AT5G55600.1 Symbols: no symbol available: no full name available. Chr5: 22522232-22524796 |
| | REVERSE LENGTH = 663 |
| 5081 | AT5G55850.2 Symbols: NOI.: Chr5: 22603617-22604951 FORWARD LENGTH = 130 |
| 5082 | AT5G55910.1 Symbols: D6PK: D6 protein kinase. Chr5: 22640055-22641634 REVERSE LENGTH = 498 |
| 5083 | AT5G55970.1 Symbols: no symbol available: no full name available. Chr5: 22668019-22669312 |
| | FORWARD LENGTH = 343 |
| 5084 | AT5G56250.2 Symbols: HAP8: HAPLESS 8. Chr5: 22768918-22771869 REVERSE LENGTH = 811 |
| 5085 | AT5G56790.1 Symbols: no symbol available: no full name available. Chr5: 22968610-22971391 |
| | FORWARD LENGTH = 669 |
| 5086 | AT5G56900.2 Symbols: no symbol available: no full name available. Chr5: 23019750-23022670 |
| | REVERSE LENGTH = 596 |
| 5087 | AT5G57015.1 Symbols: ckl12: casein kinase I-like 12. Chr5: 23071508-23074577 FORWARD |
| | LENGTH = 435 |
| 5088 | AT5G57050.1 Symbols: ABI2, AtABI2: ABA INSENSITIVE 2. Chr5: 23087720-23089303 FORWARD |
| | LENGTH = 423 |
| 5089 | AT5G57180.2 Symbols: CIA2: chloroplast import apparatus 2. Chr5: 23168393-23170763 FORWARD |
| | LENGTH = 435 |
| 5090 | AT5G57290.1 Symbols: P3B, AtP3B: ribosomal P3 protein B. Chr5: 23207049-23207835 REVERSE |
| | LENGTH = 120 |
| 5091 | AT5G57340.2 Symbols: no symbol available: no full name available. Chr5: 23228022-23229094 |
| | FORWARD LENGTH = 256 |
| 5092 | AT5G57550.1 Symbols: XTH25, XTR3: xyloglucan endotransglucosylase/hydrolase 25, xyloglucan |
| | endotransglycosylase 3. Chr5: 23305055-23306384 REVERSE LENGTH = 284 |
| 5093 | AT5G57565.1 Symbols: no symbol available: no full name available. Chr5: 23310872-23311494 |
| | FORWARD LENGTH = 143 |
| 5094 | AT5G57610.1 Symbols: no symbol available: no full name available. Chr5: 23325307-23329099 |
| | FORWARD LENGTH = 1054 |
| 5095 | AT5G57790.1 Symbols: AOG1: ABORTED GAMETOPHYTE 1. Chr5: 23412596-23413442 REVERSE |
| | LENGTH = 216 |
| 5096 | AT5G58200.2 Symbols: no symbol available: no full name available. Chr5: 23549605-23551972 |
| | FORWARD LENGTH = 309 |
| 5097 | AT5G58370.2 Symbols: EngB-3.: Chr5: 23593358-23595645 FORWARD LENGTH = 465 |
| 5098 | AT5G58540.1 Symbols: no symbol available: no full name available. Chr5: 23663400-23665182 |
| | FORWARD LENGTH = 484 |
| 5099 | AT5G58930.1 Symbols: no symbol available: no full name available. Chr5: 23794529-23796094 |
| | REVERSE LENGTH = 521 |
| 5100 | AT5G58960.1 Symbols: GIL1: GRAVITROPIC IN THE LIGHT. Chr5: 23805799-23808360 |
| | FORWARD LENGTH = 559 |
| 5101 | AT5G59010.1 Symbols: BSK5: brassinosteroid-signaling kinase 5. Chr5: 23820578-23823099 |
| | REVERSE LENGTH = 489 |
| 5102 | AT5G59370.1 Symbols: ACT4: actin 4. Chr5: 23950109-23951586 FORWARD LENGTH = 377 |
| 5103 | AT5G59430.1 Symbols: TRP1, ATTRP1: TELOMERE REPEAT BINDING PROTEIN 1, telomeric |
| | repeat binding protein 1. Chr5: 23968254-23970695 FORWARD LENGTH = 578 |
| 5104 | AT5G59780.3 Symbols: ATMYB59-1, MYB59, ATMYB59, ATMYB59-3, ATMYB59-2: MYB DOMAIN |
| | PROTEIN 59, myb domain protein 59. Chr5: 24082425-24083350 REVERSE LENGTH = 235 |
| 5105 | AT5G59950.5 Symbols: no symbol available: no full name available. Chr5: 24140235-24141504 |
| | FORWARD LENGTH = 245 |
| 5106 | AT5G60050.1 Symbols: no symbol available: no full name available. Chr5: 24183680-24185264 |
| | REVERSE LENGTH = 499 |
| 5107 | AT5G60100.2 Symbols: PRR3, APRR3: pseudo-response regulator 3. Chr5: 24198215-24200502 |
| | REVERSE LENGTH = 522 |
| 5108 | AT5G60450.1 Symbols: ARF4: auxin response factor 4. Chr5: 24308558-24312187 REVERSE |
| | LENGTH = 788 |
| 5109 | AT5G60550.1 Symbols: ATSNAK1, GRIK2: geminivirus rep interacting kinase 2. Chr5: 24340135- |
| | 24342356 FORWARD LENGTH = 407 |
| 5110 | AT5G60570.1 Symbols: no symbol available: no full name available. Chr5: 24348713-24349894 |
| | FORWARD LENGTH = 393 |
| 5111 | AT5G60580.2 Symbols: no symbol available: no full name available. Chr5: 24354298-24356706 |
| | FORWARD LENGTH = 494 |
| 5112 | AT5G60690.1 Symbols: IFL, REV, IFL1: INTERFASCICULAR FIBERLESS, INTERFASCICULAR |
| | FIBERLESS 1, REVOLUTA. Chr5: 24397734-24401933 FORWARD LENGTH = 842 |
| 5113 | AT5G60750.1 Symbols: SCO4: snowy cotyledon 4. Chr5: 24431072-24433199 FORWARD |
| | LENGTH = 347 |
| 5114 | AT5G60890.1 Symbols: ATR1, AtATR1, ATMYB34, MYB34: ALTERED TRYPTOPHAN |
| | REGULATION 1, myb domain protein 34. Chr5: 24495029-24496220 FORWARD LENGTH = 295 |
| 5115 | AT5G60940.1 Symbols: no symbol available: no full name available. Chr5: 24523092-24525655 |
| | FORWARD LENGTH = 429 |
| 5116 | AT5G61010.1 Symbols: EXO70E2, ATEXO70E2: exocyst subunit exo70 family protein E2. |
| | Chr5: 24554612-24556531 FORWARD LENGTH = 639 |
| 5117 | AT5G61230.1 Symbols: PIA2, ANK6: ankyrin repeat protein 6, phytochrome interacting ankyrin-repeat |
| | protcin 2. Chr5: 24628254-24628778 FORWARD LENGTH = 174 |
| 5118 | AT5G61380.1 Symbols: PRR1, AtTOC1, TOC1, APRR1: TIMING OF CAB EXPRESSION 1, PSEUDO- |
| | RESPONSE REGULATOR 1. Chr5: 24675540-24678176 FORWARD LENGTH = 618 |
| 5119 | AT5G61420.2 Symbols: HAG1, MYB28, AtMYB28, PMG1: HIGH ALIPHATIC GLUCOSINOLATE |
| | 1, PRODUCTION OF METHIONINE-DERIVED GLUCOSINOLATE 1, myb domain protein 28. |
| | Chr5: 24689475-24690795 REVERSE LENGTH = 366 |
| 5120 | AT5G61540.1 Symbols: no symbol available: no full name available. Chr5: 24744994-24747142 |
| | FORWARD LENGTH = 359 |
| 5121 | AT5G61570.1 Symbols: no symbol available: no full name available. Chr5: 24758507-24760201 |
| | FORWARD LENGTH = 361 |
| 5122 | AT5G61960.1 Symbols: ML1, AML1: MEI2-like protein 1. Chr5: 24879001-24883483 REVERSE |
| | LENGTH = 915 |
| 5123 | AT5G62090.1 Symbols: SLK2: SEUSS-like 2. Chr5: 24935221-24938540 REVERSE LENGTH = 816 |
| 5124 | AT5G62130.2 Symbols: no symbol available: no full name available. Chr5: 24950685-24952426 |
| | REVERSE LENGTH = 345 |
| 5125 | AT5G62620.1 Symbols: GALT6.: Chr5: 25137136-25139764 FORWARD LENGTH = 681 |
| 5126 | AT5G62640.3 Symbols: ELF5, AtELF5: EARLY FLOWERING 5. Chr5: 25149584-25152351 |
| | REVERSE LENGTH = 540 |
| 5127 | AT5G62760.1 Symbols: no symbol available: no full name available. Chr5: 25204730-25209393 |
| | REVERSE LENGTH = 661 |
| 5128 | AT5G64130.3 Symbols: no symbol available: no full name available. Chr5: 25664547-25665339 |
| | REVERSE LENGTH = 140 |
| 5129 | AT5G64150.1 Symbols: no symbol available: no full name available. Chr5: 25668997-25670731 |
| | REVERSE LENGTH = 377 |
| 5130 | AT5G64250.2 Symbols: no symbol available: no full name available. Chr5: 25697623-25698945 |
| | REVERSE LENGTH = 333 |
| 5131 | AT5G64340.1 Symbols: SAC51: SUPPRESSOR OF ACAULIS 51. Chr5: 25730890-25731936 |
| | REVERSE LENGTH = 348 |
| 5132 | AT5G64460.2 Symbols: no symbol available: no full name available. Chr5: 25773009-25775104 |
| | REVERSE LENGTH = 282 |
| 5133 | AT5G64550.1 Symbols: no symbol available: no full name available. Chr5: 25801794-25803698 |
| | REVERSE LENGTH = 634 |
| 5134 | AT5G64960.1 Symbols: CDKC2, CDKC 2: cyclin dependent kinase group C2, Cyclin-dependent kinase |
| | C 2. Chr5: 25955497-25958427 FORWARD LENGTH = 513 |
| 5135 | AT5G65180.1 Symbols: no symbol available: no full name available. Chr5: 26045625-26047806 |
| | FORWARD LENGTH = 439 |
| 5136 | AT5G65310.1 Symbols: HB5, ATHB-5, ATHB5: homeobox protein 5. Chr5: 26102457-26104217 |
| | REVERSE LENGTH = 312 |
| 5137 | AT5G65740.2 Symbols: no symbol available: no full name available. Chr5: 26301702-26303422 |
| | REVERSE LENGTH = 300 |
| 5138 | AT5G66005.3 Symbols: no symbol available: no full name available. Chr5: 26397864-26399123 |
| | REVERSE LENGTH = 192 |
| 5139 | AT5G66040.1 Symbols: STR16: sulfurtransferase protein 16. Chr5: 26410557-26411139 FORWARD |
| | LENGTH = 120 |
| 5140 | AT5G66050.1 Symbols: no symbol available: no full name available. Chr5: 26412333-26414428 |
| | REVERSE LENGTH = 340 |
| 5141 | AT5G66110.1 Symbols: HIPP27: heavy metal associated isoprenylated plant protein 27. Chr5: 26430245- |
| | 26430778 FORWARD LENGTH = 147 |
| 5142 | AT5G66250.4 Symbols: no symbol available: no full name available. Chr5: 26469050-26470376 |
| | FORWARD LENGTH = 326 |
| 5143 | AT5G66310.1 Symbols: no symbol available: no full name available. Chr5: 26485786-26490304 |
| | REVERSE LENGTH = 1063 |
| 5144 | AT5G66420.2 Symbols: no symbol available: no full name available. Chr5: 26521893-26524986 |
| | REVERSE LENGTH = 754 |
| 5145 | AT5G66600.4 Symbols: no symbol available: no full name available. Chr5: 26575105-26578315 |
| | REVERSE LENGTH = 614 |
| 5146 | AT5G66730.1 Symbols: ENY, IDD1: INDETERMINATE DOMAIN 1, ENHYDROUS. Chr5: 26641914- |
| | 26643883 REVERSE LENGTH = 500 |
| 5147 | AT5G67440.1 Symbols: MEL2, NPY3: NAKED PINS IN YUC MUTANTS 3, MAB4/ENP/NPY1-LIKE |
| | 2. Chr5: 26912947-26914906 REVERSE LENGTH = 579 |
| 5148 | AT5G67600.1 Symbols: WIH1: WINDHOSE 1. Chr5: 26959754-26960226 REVERSE LENGTH = 82 |
| 5149 | AT5G67620.1 Symbols: no symbol available: no full name available. Chr5: 26964891-26965720 |
| | REVERSE LENGTH = 182 |
| 5150 | ATMG00410.1 Symbols: ATP6, ATP6-1: ATPASE SUBUNIT 6, ATPase subunit 6-1. ChrM: 111750- |
| | 112907 FORWARD LENGTH = 385 |
| 5151 | ATMG00730.1 Symbols: COX3: cytochrome c oxidase subunit 3. ChrM: 218280-219077 FORWARD |
| | LENGTH = 265 |
| 5152 | AT4G36900.1 RAP2.10 (related to AP2 10). DNA binding. transcription factor |
| 5153 | AT2G23340.1 |
| 5154 | AT5G67190.1 Ethylene-responsive transcription factor ERF010. DEAR2, DREB and EAR MOTIF |
| | PROTEIN 2. Binds to the GCC-box pathogenesis-related promoter clement |
| 5155 | AT3G50260.1 |
| 5156 | ORF 1 frame 1 Candidate gene identified from homology search to AT1G01060 |
| 5157 | ORF 2 frame 1 Candidate gene identified from homology search to AT1G01060 |
| 5158 | ORF 3 frame 1 Candidate gene identified from homology search to AT1G01060 |
| 5159 | ORF 4 frame 1 Candidate gene identified from homology search to AT1G01060 |
| 5160 | ORF 5 frame 1 Candidate gene identified from homology search to AT1G01060 |
| 5161 | ORF 6 frame 1 Candidate gene identified from homology search to AT1G01060 |
| 5162 | ORF 7 frame 2 Candidate gene identified from homology search to AT1G01060 |
| 5163 | ORF 8 frame 2 Candidate gene identified from homology search to AT1G01060 |
| 5164 | ORF 9 frame 2 Candidate gene identified from homology search to AT1G01060 |
| 5165 | ORF 10 frame 2 Candidate gene identified from homology search to AT1G01060 |
| 5166 | ORF 11 frame 2 Candidate gene identified from homology search to AT1G01060 |
| 5167 | ORF 12 frame 2 Candidate gene identified from homology search to AT1G01060 |
| 5168 | ORF 13 frame 2 Candidate gene identified from homology search to AT1G01060 |
| 5169 | ORF 14 frame 2 Candidate gene identified from homology search to AT1G01060 |
| 5170 | ORF 15 frame 2 Candidate gene identified from homology search to AT1G01060 |
| 5171 | ORF 16 frame 3 Candidate gene identified from homology search to AT1G01060 |
| 5172 | ORF 17 frame 3 Candidate gene identified from homology search to AT1G01060 |
| 5173 | ORF 18 frame 3 Candidate gene identified from homology search to AT1G01060 |
| 5174 | ORF 19 frame 3 Candidate gene identified from homology search to AT1G01060 |
| 5175 | ORF 20 frame 3 Candidate gene identified from homology search to AT1G01060 |
| 5176 | ORF 1 frame 1 Candidate gene identified from homology search to AT1G01060 |
| 5177 | ORF 2 frame 1 Candidate gene identified from homology search to AT1G01060 |
| 5178 | ORF 3 frame 1 Candidate gene identified from homology search to AT1G01060 |
| 5179 | ORF 4 frame 1 Candidate gene identified from homology search to AT1G01060 |
| 5180 | ORF 5 frame 1 Candidate gene identified from homology search to AT1G01060 |
| 5181 | ORF 6 frame 2 Candidate gene identified from homology search to AT1G01060 |
| 5182 | ORF 7 frame 2 Candidate gene identified from homology search to AT1G01060 |
| 5183 | ORF 8 frame 2 Candidate gene identified from homology search to AT1G01060 |
| 5184 | ORF 9 frame 2 Candidate gene identified from homology search to AT1G01060 |
| 5185 | ORF 10 frame 3 Candidate gene identified from homology search to AT1G01060 |
| 5186 | ORF 11 frame 3 Candidate gene identified from homology search to AT1G01060 |
| 5187 | ORF 12 frame 3 Candidate gene identified from homology search to AT1G01060 |
| 5188 | ORF 13 frame 3 Candidate gene identified from homology search to AT1G01060 |
| 5189 | ORF 1 frame 1 Candidate gene identified from homology search to AT1G01060 |
| 5190 | ORF 2 frame 1 Candidate gene identified from homology search to AT1G01060 |
| 5191 | ORF 3 frame 1 Candidate gene identified from homology search to AT1G01060 |
| 5192 | ORF 4 frame 1 Candidate gene identified from homology search to AT1G01060 |
| 5193 | ORF 5 frame 1 Candidate gene identified from homology search to AT1G01060 |
| 5194 | ORF 6 frame 1 Candidate gene identified from homology search to AT1G01060 |
| 5195 | ORF 7 frame 1 Candidate gene identified from homology search to AT1G01060 |
| 5196 | ORF 8 frame 1 Candidate gene identified from homology search to AT1G01060 |
| 5197 | ORF 9 frame 2 Candidate gene identified from homology search to AT1G01060 |
| 5198 | ORF 10 frame 2 Candidate gene identified from homology search to AT1G01060 |
| 5199 | ORF 11 frame 2 Candidate gene identified from homology search to AT1G01060 |
| 5200 | ORF 12 frame 2 Candidate gene identified from homology search to AT1G01060 |
| 5201 | ORF 13 frame 2 Candidate gene identified from homology search to AT1G01060 |
| 5202 | ORF 14 frame 2 Candidate gene identified from homology search to AT1G01060 |
| 5203 | ORF 15 frame 3 Candidate gene identified from homology search to AT1G01060 |
| 5204 | ORF 16 frame 3 Candidate gene identified from homology search to AT1G01060 |
| 5205 | ORF 17 frame 3 Candidate gene identified from homology search to AT1G01060 |
| 5206 | ORF 18 frame 3 Candidate gene identified from homology search to AT1G01060 |
| 5207 | ORF 19 frame 3 Candidate gene identified from homology search to AT1G01060 |
| 5208 | ORF 20 frame 3 Candidate gene identified from homology search to AT1G01060 |
| 5209 | ORF 21 frame 3 Candidate gene identified from homology search to AT1G01060 |
| 5210 | ORF 22 frame 3 Candidate gene identified from homology search to AT1G01060 |
| 5211 | ORF 1 frame 1 Candidate gene identified from homology search to At4g36900.1 |
| 5212 | ORF 2 frame 1 Candidate gene identified from homology search to At4g36900.1 |
| 5213 | ORF 3 frame 1 Candidate gene identified from homology search to At4g36900.1 |
| 5214 | ORF 4 frame 1 Candidate gene identified from homology search to At4g36900.1 |
| 5215 | ORF 5 frame 1 Candidate gene identified from homology search to At4g36900.1 |
| 5216 | ORF 6 framc 1 Candidate gene identified from homology search to At4g36900.1 |
| 5217 | ORF 7 frame 2 Candidate gene identified from homology search to At4g36900.1 |
| 5218 | ORF 8 frame 2 Candidate gene identified from homology search to At4g36900.1 |
| 5219 | ORF 9 frame 2 Candidate gene identified from homology search to At4g36900.1 |
| 5220 | ORF 10 frame 2 Candidate gene identified from homology search to At4g36900.1 |
| 5221 | ORF 11 frame 2 Candidate gene identified from homology search to At4g36900.1 |
| 5222 | ORF 12 frame 2 Candidate gene identified from homology search to At4g36900.1 |
| 5223 | ORF 13 frame 3 Candidate gene identified from homology search to At4g36900.1 |
| 5224 | ORF 14 frame 3 Candidate gene identified from homology search to At4g36900.1 |
| 5225 | ORF 15 frame 3 Candidate gene identified from homology search to At4g36900.1 |
| 5226 | ORF 16 frame 3 Candidate gene identified from homology search to At4g36900.1 |
| 5227 | ORF 17 frame 3 Candidate gene identified from homology search to At4g36900.1 |

Gene Ontology Analysis of the Candidate uORF Genes

The gene list was analyzed with the DAVID bioinformatics resource version 6.8 (david.ncifcrf.gov/). Table 2 summarizes the output of this analysis. Of the functional categories analyzed the keywords captured 99.4% of the unique genes. Of these, a large proportion include one or more of the terms alternative splicing, transcription factor, transcription, nucleus, DNA binding, coiled-coil and kinase. Of the Gene ontology analysis, biological process categories enriched for transcription (DNA template), regulation of transcription (DNA template) protein phosphorylation and kinase were also notable. It was also surprising to note that 18 genes involved in the response to ethylene were detected and 20 genes involved in floral development. Of the cellular function categorization, the largest proportion of genes 46.9% were assigned to the nucleus. On the molecular function categorization, transcription factors, protein kinase, DNA binding and ATP binding were all identified as enriched in the uORF list generated.

TABLE 2
Output of gene list analyzed with DAVID bioinformatics resource

| | Detected | Detected in | |
|---|---|---|---|
| | in all three | at least two | Detected only in |
| Unique | datasets | datasets | one dataset |
| 24 | 45 | 12 | 5 |

Numbering Convention, Transcription Factors and uPEPs

The uORFs and uPEPs identified in this project are included in the Sequence Listing as SEQ ID NO: 2n−1 (SEQ ID NO: 1, 3, 5, 7, 9, and every other odd numbered sequence to 3997) and SEQ ID No: 2n (SEQ ID NOs: 2, 4, 6, 8, 10, and every other even numbered sequence to 3998), respectively, where n=1-1999 (i.e., the identified uORFs are SEQ ID NO: 1, 3, 5, 7, 9, and every other odd numbered sequence to 3997; and the identified uORF translations are SEQ ID NO: 2, 4, 6, 8, 10, and every other even numbered sequence to 3998, respectively). These and a description for each of the transcription factor classes are included the Sequence Listing; the summary of each sequence includes AGI number, TAIR_ID, uORF ‘5 STOP, uORF 3′ STOP, leader length, and each is followed by the uORF or uPEP (the latter is a translation of the uORF).

A uORF and uORF translation (uPEP) analysis was conducted with a total of 130 transcription factors encoding loci from Arabidopsis identified in our analysis. Representatives from almost all transcription factor classes were identified by our analysis, but some families seem to be particularly enriched in uORFs; these include the AP2 gene family, the homeodomain leucine zipper family, the sNF-family and one STAT transcription factor. The gene ontology list of genes involved in flowering is included in the Sequence Listing which contains a number of transcription factors including AP2, ARF, Homeodomain, and MYB transcription factors. It is also worth noting that the flowering time control protein FCA, which is theorized to function as an RNA binding protein is included in the list of uORF regulated loci that we identified.

### Example Individual Gene Profiles

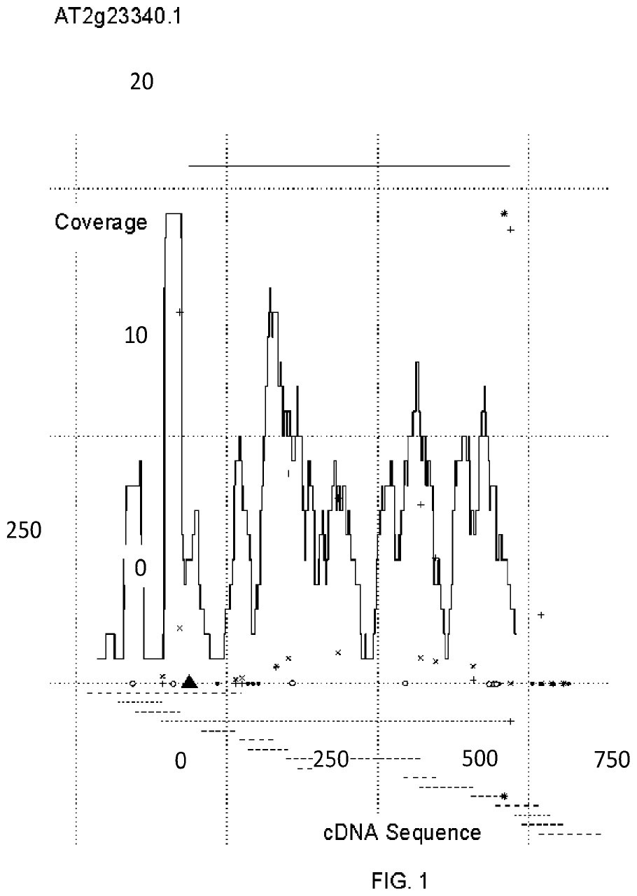

Below are more detailed descriptions for a selection of example loci where the ribosome coverage, stop-stop fragment and statistically significant ratios and deltas (based on ribosome profiling before and after the 3′ stop) are shown in FIGS. 1-3.

At2g23340.1 two clear clusters of ribosomes are found upstream of the long ORF (horizontal line at top of FIG. 1). There is a long ORF (top dotted line: - - - - - -) covering the leader region. The uORF designated by open circles was selected as significantly different by ratio (FIG. 1).

At4g36900.1: FIG. 2 shows a clear enrichment of ribosomes apparent in the leader (selected as significantly different by ratio and delta) (FIG. 2).

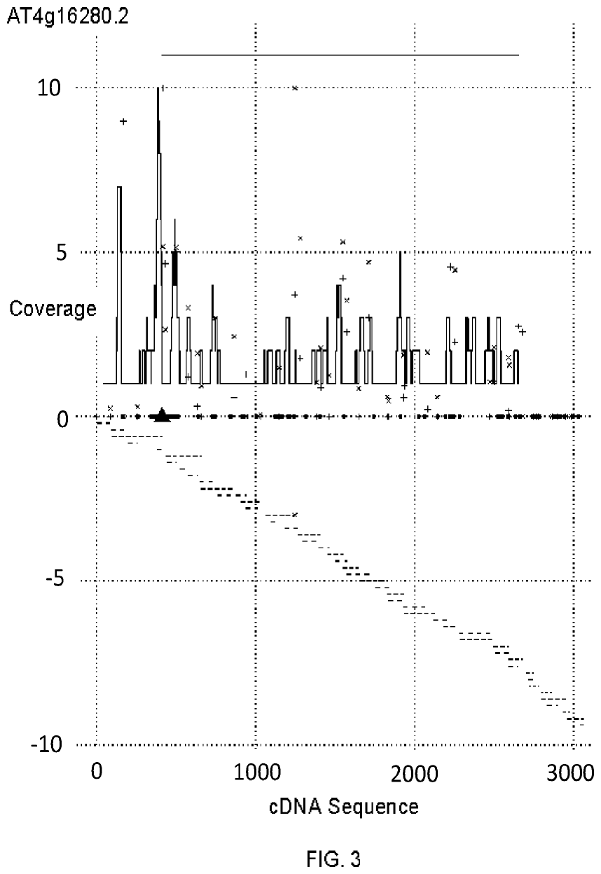

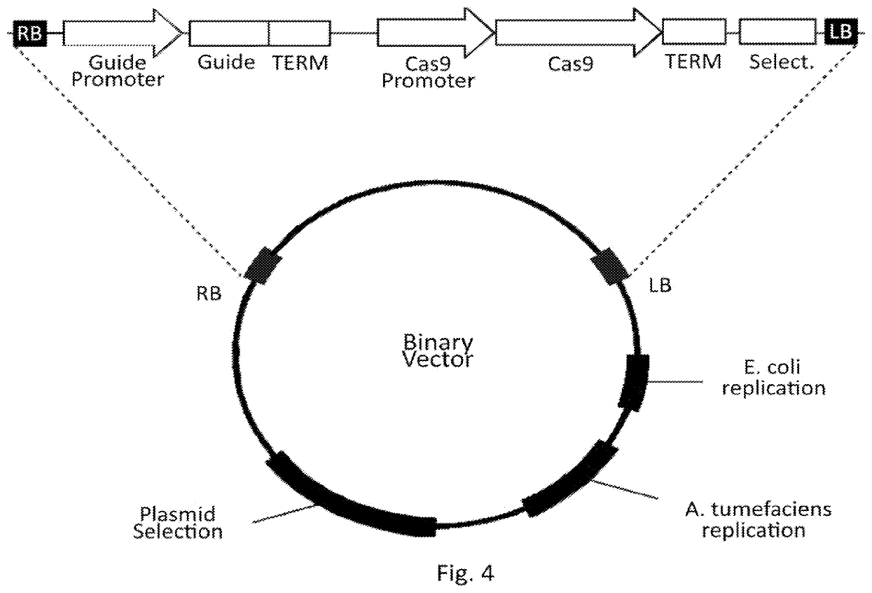

AT4g16280.2 is the functional gene model for FCA. There are four gene models but only 0.2 and 0.4 contain open reading frames and the alternative splicing could potentially interfere with the candidate uORF that is clearly identified as the enrichment of ribosomes upstream of the long ORF (FIG. 3).

HD-ZIP transcription factors. This class of transcription factors is especially enriched for uORFs. FIGS. 1, 2, and 3 highlight some of the gene candidates with ribosome profiling demonstrating ribosome enrichment upstream of the long open reading frame.

### Bioinformatic Analysis and the Identification of Homologs

An important aspect of the present invention is that if a locus containing an mORFs that encodes a polypeptide with a desired function is identified as having an upstream uORF in a reference species, the equivalent locus with an mORF encoding a homolog of the polypeptide in a target crop will also typically possess a uORF. That is, the presence of uORF is typically conserved across homologous loci An example of this phenomenon has been shown with ascorbate biosynthesis genes across species. For example, see Zhang et al., 2018, Nature Biotechnology volume 36, pages 894-898. Thus, a practitioner may apply the methods herein to identify a uORF in a locus in Arabidopsis, and then identify loci with main ORFs encoding homologs in the target crop and then deploy gene editing to the mutate the uORF sequence upstream of the mORF in the crop to remove the repression imposed by the uORF. Typically, this involves mutating sequences between 1-1100 bp upstream of the start codon of the mORF. A single edit may be sufficient for this purpose although two edits or multiple edits can be made to the uORF.

Homologs may be identified through various bioinformatic methods, as exemplified herein. The present invention may be an integrated system, computer or computer readable medium that comprises an instruction set for determining the identity of one or more sequences in a database. In addition, the instruction set can be used to generate or identify sequences that meet any specified criteria. Furthermore, the instruction set may be used to associate or link certain functional benefits, such improved characteristics, with one or more identified sequence.

For example, the instruction set can include, e.g., a sequence comparison or other alignment program, e.g., an available program such as, for example, the Wisconsin Package Version 10.0, such as BLAST, FASTA, PILEUP, FINDPATTERNS or the like (GCG, Madison, WI). Public sequence databases such as GenBank, EMBL, Swiss-Prot and PIR or private sequence databases can be searched.

Alignment of sequences for comparison can be conducted by the local homology algorithm of Smith and Waterman (1981) Adv. Appl. Math. 2: 482-489, by the homology alignment algorithm of Needleman and Wunsch (1970) J. Mol. Biol. 48: 443-453, by the search for similarity method of Pearson and Lipman (1988) Proc. Natl. Acad. Sci. 85: 2444-2448, by computerized implementations of these algorithms. After alignment, sequence comparisons between two (or more) polynucleotides or polypeptides are typically performed by comparing sequences of the two sequences over a comparison window to identify and compare local regions of sequence similarity. The comparison window can be a segment of at least about 10 contiguous positions, usually about 50 to about 200, more usually about 100 to about 150 contiguous positions. A description of the method is provided in Ausubel, et al. supra.

A variety of methods for determining sequence relationships can be used, including manual alignment and computer assisted sequence alignment and analysis. This latter approach is a preferred approach in the present invention, due to the increased throughput afforded by computer assisted methods. As noted above, a variety of computer programs for performing sequence alignment are available, or can be produced by one of skill.

One example algorithm that is suitable for determining percent sequence identity and sequence similarity is the BLAST algorithm, which is described in Altschul, et al. (1990) J. Mol. Biol. 215: 403-410. Software for performing BLAST analyses is publicly available, e.g., through the National Library of Medicine's National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff (1992) Proc. Natl. Acad. Sci. 89: 10915-10919). Unless otherwise indicated, “sequence identity” here refers to the % sequence identity generated from a tblastx using the NCBI version of the algorithm at the default settings using gapped alignments with the filter “off” (see, for example, NIH NLM NCBI website at www.ncbi.nlm.nih.gov/, supra).

In addition to calculating percent sequence identity, the BLAST algorithm also performs a statistical analysis of the similarity between two sequences (see, e.g., Karlin and Altschul (1993) Proc. Natl. Acad. Sci. 90: 5873-5787). One measure of similarity provided by the BLAST algorithm is the smallest sum probability (P(N), which provides an indication of the probability by which a match between two nucleotide or amino acid sequences would occur by chance. For example, a nucleic acid is considered similar to a reference sequence (and, therefore, in this context, homologous) if the smallest sum probability in a comparison of the test nucleic acid to the reference nucleic acid is less than about 0.1, or less than about 0.01, and or even less than about 0.001. An additional example of a useful sequence alignment algorithm is PILEUP. PILEUP creates a multiple sequence alignment from a group of related sequences using progressive, pairwise alignments. The program can align, e.g., up to 300 sequences of a maximum length of 5,000 letters.

The integrated system, or computer, typically includes a user input interface allowing a user to selectively view one or more sequence records corresponding to the one or more character strings, as well as an instruction set which aligns the one or more character strings with each other or with an additional character string to identify one or more region of sequence similarity. The system may include a link of one or more character strings with a particular phenotype or gene function. Typically, the system includes a user readable output element that displays an alignment produced by the alignment instruction set.

The methods of this invention can be implemented in a localized or distributed computing environment. In a distributed environment, the methods may be implemented on a single computer comprising multiple processors or on a multiplicity of computers. The computers can be linked, but more preferably the computer(s) are nodes on a network. The network can be a generalized or a dedicated local or wide-area network and, in certain preferred embodiments, the computers may be components of an intra-net or an internet, or “cloud” computing platforms like that offered by Amazon Web Services.

Thus, the invention provides methods for identifying a sequence similar or homologous to one or more polynucleotides as noted herein, or one or more target polypeptides encoded by the polynucleotides, or otherwise noted herein and may include linking or associating a given phenotype such as the capacity for cellular biosynthesis of a target molecule with a sequence. In the methods, a sequence database is provided (locally or across an inter or intranet) and a query is made against the sequence database using the relevant sequences herein and associated phenotypes or functions in the cellular biosynthesis of target molecules.

Any sequence herein can be entered into the database, before or after querying the database. This provides for both expansion of the database and, if done before the querying step, for insertion of control sequences into the database. The control sequences can be detected by the query to ensure the general integrity of both the database and the query. As noted, the query can be performed using a web browser-based interface. For example, the database can be a centralized public database such as GenBank, or a private database, and the querying can be done from a remote terminal or computer across an internet or intranet.

Any sequence herein can be used to identify a similar, homologous sequence in a genome such as a target plant genome by the methods identified above. A homologous polynucleotide sequence that has a conserved or equivalent functionality in delivering a desired trait typically has at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% sequence identity to a polynucleotide sequence encoding a full length polypeptide or the full length of a conserved domain of the full length polypeptide that has been shown to produce a desired trait when overexpressed or knocked out.

### Generation of Genetically Modified Plant Cells and Plants

Genetically modified cells, plant cells, plant explants, plant tissues plant organs or plants incorporating the polynucleotides of the invention and/or expressing the polypeptides of the invention can be produced by a variety of well-established techniques. Following construction of a transformation vector, most typically an expression cassette, including one or more polynucleotides from the invention, or a segment thereof, standard techniques can be used to introduce the polynucleotide into a cell to create a genetically modified cell or cell line. Optionally, the genetically modified plant cell can be regenerated to produce an explant, tissue, or transgenic plant.

Transformation and multiplication and/or regeneration of cells is now routine, and the selection of the most appropriate transformation technique will be determined by the practitioner. The choice of method will vary with the organism to be transformed: those skilled in the art will recognize the suitability of particular methods for given organism types. Suitable methods can include, but are not limited to: electroporation of protoplasts; liposome-mediated transformation; polyethylene glycol (PEG) mediated transformation; Li-mediated transformation, transformation using, for example, viruses; micro-injection of cells; micro-projectile bombardment of cells; vacuum infiltration; or Agrobacterium tumefaciens mediated transformation. In this invention, transformation involves introducing a recombinant nucleotide sequence into a host cell in a manner to cause stable or transient expression of the sequence so as to result in expression of the encoded polypeptide which in turn results in the production of a desired target molecule.

Following transformation, genetically modified cells may be preferably selected using a dominant selectable marker incorporated into the transformation vector. Typically, such a marker will confer antibiotic or herbicide resistance on the transformed cells, and selection of transformants can be accomplished by exposing the cells to appropriate concentrations of the antibiotic or herbicide. Alternatively, color-based markers such as GFP or GUS may be used to select transformed cells, or transformed cells may be selected based on detection of expression of the polynucleotide in the introduced expression cassette by RT-PCR or detection of a target molecule produced by the genetically modified cells.

Methodologies for the generation of genetically modified plants, including transgenic plants have been reviewed by Keshavareddy et al., 2018, Int. J. Cuff. Microbiol. App. Sci (2018) 7(7): 2656-2668. Detailed methodologies have also been published in U.S. Pat. No. 7,345,217 (Zhang, Mar. 18, 2008), U.S. Pat. No. 7,511,190 (Creelman, et al., Mar. 31, 2009), U.S. Pat. No. 7,196,245 (Jiang, et al., Mar. 27, 2007) and U.S. Pat. No. 7,663,025 (Heard, et al., Feb. 16, 2010).

### Introduction of Targeted Genetic Modifications Through Gene Editing

A preferred method of practicing the invention is to use genome editing to produce a “targeted genetic modification” as referenced herein. The terms “genome editing”, “genome edited”, “genome modified”, “genetically modified” are used interchangeably to describe plants with specific DNA sequence changes in their genomes wherein those DNA sequence changes include changes of specific nucleotides, the deletion of specific nucleotide sequences or the insertion of specific nucleotide sequences.

As used herein, a technique for introducing a “targeted genetic modification” refers to any method, protocol, or technique that allows the precise and/or targeted editing at a specific location (also referred to a “locus” or “native locus” in a genome of a plant (i.e., the editing is largely or completely non-random) using a site-specific nuclease, such as a meganuclease, a zinc-finger nuclease (ZFN), an RNA-guided endonuclease (e.g., the CRISPR/Cas9 system), a TALE-endonuclease (TALEN), a recombinase, or a transposase. CRISPR is an acronym for clustered, regularly interspaced, short, palindromic repeats and Cas an abbreviation for CRISPR-associated protein; for a review, see Khandagal and Nadal, Plant Biotechnol. Rep., 2016, 10, 327. Engineered meganucleases, zinc finger nucleases (ZFN), transcription activator-like effector nucleases (TALENs) can also be used. US Patent Application 2016/0032297 provides detailed methodology for these methods. Another gene editing methodology that can be applied uses so-called ARCUS nucleases which leverage the properties of a naturally occurring gene editing enzyme—the homing endonuclease I-CreI—which evolved in nature to make a single, highly specific DNA edit before using its built-in safety switch to shut itself off.

Genome editing tools can accurately change the architecture of a genome at specific target locations. These tools can be efficiently used for the generation of plants with high crop yields, desired alterations in composition, and resistance to biotic and abiotic stresses. It may be challenging to achieve all desired modifications using a particular genome editing tool. Thus, multiple genome editing tools have been developed to facilitate efficient genome editing. Some of the major genome editing tools used to edit plant genomes are: Homologous recombination (HR), zinc finger nucleases (ZFNs), transcription activator-like effector nucleases (TALENs), pentatricopeptide repeat proteins (PPRs), the CRISPR/Cas9 system, RNA interference (RNAi), cisgenesis, and intragenesis. In addition, site-directed sequence editing and oligonucleotide-directed mutagenesis have the potential to edit the genome at the single-nucleotide level. Recently, adenine base editors (ABEs) have been developed to mutate A-T base pairs to G-C base pairs. ABEs use deoxyadeninedeaminase (TadA) with catalytically impaired Cas9 nickase to mutate A-T base pairs to G-C base pairs. A summary of these methods an applicability is provided by Mohanta et al., Genes (Basel). 2017 December; 8(12): 399.

Such genome editing methods encompass a wide range of approaches to precisely remove genes, gene fragments, to alter the DNA sequence of coding sequences or control sequences, or to insert new DNA sequences into genes or protein coding regions to reduce or increase the expression of target genes in plant genomes (Belhaj, K. 2013, Plant Methods, 9, 39; Khandagale and Nadal, 2016, Plant Biotechnol Rep, 10, 327). Preferred methods involve the in vivo site-specific cleavage to achieve double stranded breaks in the genomic DNA of the plant genome at a specific DNA sequence using nuclease enzymes and the host plant DNA repair system. Multiple approaches are available for producing double stranded breaks in genomic DNA, and thus achieve genome editing, including the use of the CRISPR/Cas system.

An extensive overview of the CRISPR/Cas system and useful applications thereof can be found at www.addgene.org/guides/crispr/

The CRISPR/Cas genome editing system provides flexibility in targeting specific sequences for modification within the genome and enables the execution of a range of different edits including the activation or upregulation of target loci or the knock-out of target loci. The method relies on providing the Cas enzyme and a short guide RNA “gRNA” containing a short guide sequence (˜20 bp), with sequence complementarity to the target DNA sequence in the plant genome. Depending on the type of Cas enzyme, alternatively a DNA, an RNA/DNA hybrid, or a double stranded DNA guide polynucleotide can be used. The guide portion of this guide polynucleotide directs the Cas enzyme to the desired cut site for cleavage with a recognition sequence for binding the Cas enzyme.

The target in the plant genome can be any ˜20 nucleotide DNA sequence, provided that the sequence is unique compared to the rest of the genome and also that the target is present immediately adjacent to a Protospacer Adjacent Motif (PAM). The PAM sequence serves as a binding signal for Cas9, but the exact sequence depends on which Cas protein is being used. A list of Cas proteins and PAM sequences can be found at www.addgene.org/guides/crispr/#pam-table

The simplest application of CRISPR/Cas is to produce knockout or loss of function alleles in a target locus. The gRNA targets the Cas enzyme to a specific locus in the genome, which then produces a double stranded break. The resulting DSB is then repaired by one of the general repair pathways present in the cell. This typically causes small nucleotide insertions or deletions (indels) at the DSB site. In most cases, small indels in the target DNA result in amino acid deletions, insertions, or frameshift mutations leading to premature stop codons within the open reading frame (ORF) of the targeted gene. The ideal result is a loss-of-function mutation within the targeted gene. However, the strength of the knockout phenotype for a given mutant cell must be validated experimentally, for example for testing for the presence of transcript from the target ORF by RT-PCR or hybridization-based approaches. These features make the CRISPR/Cas system a suitable tool for knockout of uORFs.

CRISPR/Cas can also be used to produce more sophisticated changes to the native sequence at targeted loci in the genome. This can involve inserting sequences, replacing sequences or editing specific bases so as to insert or create new domains within a polypeptide encoded at a desired locus. One way to introduce such changes is to make use of the high fidelity but low efficiency high fidelity homology directed (HDR) repair pathway within the cell. In order to make such precise modifications using HDR, a DNA repair template incorporating the desired genome modification that the practitioner desires to create at the target locus must be delivered into the cell type of interest with the gRNA(s) and Cas9 or Cas9 nickase. The repair template must contain the desired edit as well as additional homologous sequence immediately upstream and downstream of the target (termed left and right homology arms). The length of each homology arm is dependent on the size of the change being introduced, with larger insertions requiring longer homology arms. Since the efficiency of Cas9 cleavage is relatively high and the efficiency of HDR is relatively low, a large portion of the Cas9-induced DSBs will be repaired to produce edits not comprising the specific desired change. Thus, an additional confirmation/screening step is required to select one of more cells from the edited population that contain the desired change. These cells then can be regenerated into a population of cells, a tissue, organ or whole plant or plant population. Such selection can be achieved by incorporating a marker sequence into the edit, which is readily screened or by PCR or hybridization-based methods.

CRISPR-related gene editing systems can also be deployed to change specific bases without the need for double stranded breaks. Such approaches are referred to in the art as “base editing” systems. Base editing enables the irreversible conversion of a specific DNA base into another at a targeted genomic locus, for example conversing C to T, or A to G. Unlike other genome-editing tools, base editing can be achieved without double-strand breaks. When introducing a point mutation at a target locus, base editing is more efficient than traditional genome editing techniques. Since many genetic diseases arise from point mutations, base editing has important applications in disease research. Using these systems, the skilled practitioner can create a targeted genetic modification comprising an amino acid substitution or the creation of start or stop codon.

To avoid relying on HDR, which has low efficiency, researchers have developed two classes of base editors: cytosine base editors (CBEs) and adenine base editors (ABEs). Cytosine base editors are created by fusing Cas9 nickase or catalytically inactive “dead” Cas9 (dCas9) to a cytidine deaminase like APOBEC. As with traditional CRISPR techniques, base editors are targeted to a specific locus by a gRNA, and they can convert cytidine to uridine within a small editing window near the PAM site. Uridine is subsequently converted to thymidine through base excision repair, creating a C to T change. Likewise, adenosine base editors have been engineered to convert adenosine to inosine, which is treated like guanosine by the cell, creating an A to G change.

Adenine DNA deaminases do not exist in nature, but these enzymes have been created by directed evolution of the Escherichia coli TadA, a tRNA adenine deaminase. Like cytosine base editors, the evolved TadA domain is fused to a Cas9 protein to create the adenine base editor. Both types of base editors are available with multiple Cas9 variants including high fidelity Cas9's. Further advancements have been made by optimizing expression of the fusions, modifying the linker region between Cas variant and deaminase to adjust the editing window, or adding fusions that increase product purity such as the DNA glycosylase inhibitor (UGI) or the bacteriophage Mu-derived Gam protein (Mu-GAM).

While many base editors are designed to work in a very narrow window proximal to the PAM sequence, some base editing systems create a wide spectrum of single-nucleotide variants (somatic hypermutation) in a wider editing window, and are thus well suited to directed evolution applications. Examples of these base editing systems include targeted AID-mediated mutagenesis (TAM) and CRISPR-X, in which Cas9 is fused to activation-induced cytidine deaminase (AID).

Other CRISPR systems, specifically the Type VI CRISPR enzymes Cas13a/C2c2 and Cas13b, target RNA rather than DNA. Fusing a hyperactive adenosine deaminase that acts on RNA, ADAR2(E488Q), to catalytically dead Cas13b creates a programmable RNA base editor that converts adenosine to inosine in RNA (termed REPAIR). Since inosine is functionally equivalent to guanosine, the result is an A->G change in RNA. The catalytically inactive Cas13b ortholog from Prevotella sp., dPspCas13b, does not appear to require a specific sequence adjacent to the RNA target, making this a very flexible editing system. Editors based on a second ADAR variant, ADAR2(E488Q/T375G), display improved specificity, and editors carrying the delta-984-1090 ADAR truncation retain RNA editing capabilities and are small enough to be packaged in AAV particles.

In the context of this description, it is recognized that the term Cas nuclease includes any nuclease which site-specifically recognizes CRISPR sequences based on gRNA or DNA sequences and includes Cas9, Cpf1 and others described below. Many authors have identified that CRISPR/Cas genome editing, is a preferred way to edit the genomes of complex organisms (Sander and Joung, 2013, Nat Biotech, 2014, 32, 347; Wright et al., 2016, Cell, 164, 29) including plants (Zhang et al., 2016, Journal of Genetics and Genomics, 43, 151; Puchta 2016, Plant J., 87, 5; Khandagale and Nadaf, 2016, Plant Biotechnol. Rep., 10, 327). US Patent Application 2016/020822 provides extensive description of the materials and methods useful for genome editing in plants using the CRISPR/Cas9 system and describes many of the uses of the CRISPR/Cas9 system for genome editing of a range of gene targets in crops.

It is further recognized that many variations of the CRISPR/Cas system can be used for applying the invention herein, including the use of wild-type Cas9 from Streptococcus pyogenes (Type II Cas) (Barakate and Stephens, 2016, Frontiers in Plant Science, 7, 765; Bortesi and Fischer, 2015, Biotechnology Advances 5, 33, 41; Cong et al., 2013, Science, 339, 819; Rani et al., 2016, Biotechnology Letters, 1-16; Tsai et al., 2015, Nature biotechnology, 33, 187). Other examples include Tru-gRNA/Cas9 in which off-target mutations are significantly decreased (Fu et al., 2014, Nature biotechnology, 32, 279; Osakabe et al., 2016, Scientific Reports, 6, 26685; Smith et al., 2016, Genome biology, 17, 1; Zhang et al., 2016, Scientific Reports, 6, 28566), a high specificity Cas9 (mutated S. pyogenes Cas9) with little to no off target activity (Kleinstiver et al., 2016, Nature 529, 490; Slaymaker et al., 2016, Science, 351, 84). Further variations comprise the Type I and Type III systems in which multiple Cas proteins are expressed to achieve editing (Li et al., 2016, Nucleic acids research, 44:e34; Luo et al., 2015, Nucleic acids research, 43, 674), the Type V Cas system using the Cpf1 enzyme (Kim et al., 2016, Nature biotechnology, 34, 863; Toth et al., 2016, Biology Direct, 11, 46; Zetsche et al., 2015, Cell, 163, 759), DNA-guided editing using the NgAgo Argonaute enzyme from Natronobacterium gregoryi that employs guide DNA (Xu et al., 2016, Genome Biology, 17, 186), and the use of a two vector system in which Cas9 and gRNA expression cassettes are carried on separate vectors (Cong et al., 2013, Science, 339, 819). A unique nuclease Cpf1, an alternative to Cas9 has advantages over the Cas9 system in reducing off-target edits which creates unwanted mutations in the host genome. Examples of crop genome editing using the CRISPR/Cpf1 system include rice (Tang et. al., 2017, Nature Plants 3, 1-5; Wu et. al., 2017, Molecular Plant, Mar. 16, 2017) and soybean (Kim et., al., 2017, Nat Commun. 8, 14406). Other authors have described the use of Argonaute related proteins as an alternative to CRISPR systems for gene editing (Hegge et al. Nature Rev. Microbiol. 2017. Epub 2017/07/25. pmid: 28736447; Swarts et al. Nucleic Acids Res. 2015; 43(10):5120-9. Epub 2015/05/01. pmid: 25925567; Swarts et al. Nature. 2014; 507(7491):258-61. Epub 2014/02/18. pmid: 24531762. See also PCT Application Number PCT/US2019/025163 and/or Publication Number WO2019204266A1.

Detailed methodologies for gene editing in plants to create new crop traits, including the selection of cells containing the desired edits, and methods for introducing the CRISPR system components into an initial target plant cell are set forth in published patent application WO2019195157. As specified therein, the “guide polynucleotide” in a CRISPR system also relates to a polynucleotide sequence that can form a complex with a Cas endonuclease and enables the Cas endonuclease to recognize and optionally cleave a DNA target site. The guide polynucleotide can be a single molecule (i.e., a single guide RNA (gRNA) that is a synthetic fusion between a crRNA and part of the tracrRNA sequence) or two molecules (i.e., the crRNA and tracrRNA as found in natural Cas9 systems in bacteria). The guide polynucleotide sequence can be provided as an RNA sequence or can be transcribed from a DNA sequence to produce an RNA sequence. The guide polynucleotide sequence can also be provided as a combination RNA-DNA sequence (see for example, Yin, H. et al., 2018, Nature Chemical Biology, 14, 311). As used herein “guide RNA” sequences comprise a variable targeting domain, called the “guide”, complementary to the target site in the genome, and an RNA sequence that interacts with the Cas9 or Cpf1 endonuclease, called the “guide RNA scaffold”. A guide polynucleotide that solely comprises ribonucleic acids is also referred to as a “guide RNA”. As used herein the “guide target sequence” refers to the sequence of the genomic DNA adjacent to a PAM site, where the gRNA will bind to cleave the DNA. The “guide target sequence” is often complementary to the “guide” portion of the gRNA, however several mismatches, depending on their position, can be tolerated and still allow Cas mediated cleavage of the DNA. The method also provides introducing single guide RNAs (gRNAs) into plants. The single guide RNAs (gRNAs) include nucleotide sequences that are complementary to the target chromosomal DNA. The gRNAs can be, for example, engineered single chain guide RNAs that comprise a crRNA sequence (complementary to the target DNA sequence) and a common tracrRNA sequence, or as crRNA-tracrRNA hybrids. The gRNAs can be introduced into the cell or the organism as a DNA with an appropriate promoter, as an in vitro transcribed RNA, or as a synthesized RNA. Basic guidelines for designing the guide RNAs for any target gene of interest are well known in the art as described for example by Brazelton et al. (Brazelton, V. A. et al., 2015, GM Crops & Food, 6, 266-276) and Zhu (Zhu, L. J. 2015, Frontiers in Biology, 10, 289-296).

Published patent applications WO2019195157 and WO2019204266A1 also provides example of the types of mutation that can lead to increased activity of transcription factor polypeptides. These include mutations to the coding sequence that give rise to amino acid changes in the encoded protein.

In certain preferred embodiments of the present invention, the guide polynucleotide/Cas endonuclease system can be used to allow for the insertion of a promoter or promoter element, such as an enhancer element, of any one the transcription factor sequences of the invention, wherein the promoter insertion (or promoter element deletion) results in any one of the following or any one combination of the following: a permanently activated gene locus, an increased promoter activity (increased promoter strength), an increased promoter tissue specificity, a decreased promoter tissue specificity, a new promoter activity, an extended window of gene expression, a modification of the timing or developmental progress of gene expression, a mutation of DNA binding elements and/or an addition of DNA binding elements.

The guide RNA/Cas endonuclease system can be used to allow for the insertion of a promoter element to increase the expression of the transcription factor sequences of the invention. Promoter elements, such as enhancer elements, are often introduced in promoters driving gene expression cassettes in multiple copies for trait gene testing or to produce transgenic plants expressing specific traits. Enhancer elements can be, but are not limited to, a 35S enhancer element (Benfey et al, EMBO J., 1989; 8: 2195-2202). In some plants (events), the enhancer elements can cause a desirable phenotype, a yield increase, or a change in expression pattern of the trait of interest that is desired. It may be desired to remove the extra copies of the enhancer element while keeping the trait gene cassettes intact at their integrated genomic location. The guide RNA/Cas endonuclease can be used to remove the unwanted enhancing element from the plant genome. A guide RNA can be designed to contain a variable targeting region targeting a target site sequence of 12-30 bps adjacent to a NGG (PAM) in the enhancer. The Cas endonuclease can make cleavage to insert one or multiple enhancers.

To repress the function of a target uORF and activate the downstream mORF, bases can be deleted from the uORF, or additional stop codons can be created. Other mutations or edits that may be used to repress the function of a target uORF include mutations of the start ATG codon, amino acid deletions, insertions, or frameshift mutations leading to premature stop codons or any of a number of deleterious mutations within the uORF. In some cases, it may be optimum to substitute bases within a uORF to produce an optimized phenotype whereby an increased yield, increased stress tolerance, or altered biochemical composition may be obtained without substantial off types such as, for example, organ abnormalities or dwarfing.

Delivery of Gene Editing Components into Plant Cells and Plants:

Sandhya et al. 2020. J. Genet. Eng. Biotechnol. 18: 25. Published online 2020 Jul. 7. doi: 10.1186/s43141-020-00036-8, present methods for delivering gene editing tools such CRISPR/Cas9 components into plants to execute the gene editing process. The effective delivery of CRISPR/Cas9 components, including the guide sequence, the CAS9, and where applicable a DNA-repair template containing the desired sequence edit, into plant cells is critical for editing to be efficient. The practitioner can select from a variety of delivery methods to introduce the gene editing components into plant cells. These include Agrobacterium-mediated transformation, bombardment or biolistic methods of transformation, floral-dip, and PEG-mediated protoplast transformation. Additional methods include nanoparticle and pollen magnetofection-mediated delivery systems (Kwak et ai., 2019, Nature Nanotechnology, DOI 10.1038/S41565-019-0375-4) (Demirer et al, 2019, Nature Nanotechnology, DOI 10.1038/S41565-019-0382-5) can be used. CRISPR constructs can be coated onto gold particles for gene gun mediated introduction into plant cells, CRISPR constructs can be transfected into protoplasts using PEG, or introduced via an Agrobacterium strain harboring a CRISPR vector. Components may also be introduced via floral dip (Castel et al., 2019. PLoS One 14:e0204778) or a pollen-tube tube pathway-based method. In the next step, a plant or plant cell containing a targeted genetic modification produced by the introduced CRISPR system is selected. This may involve regenerating a cell containing the modification into an explant, a plant tissue, or whole plant. In some instances, this procedure involves selecting explants harboring the genome edit on selection plates and regenerating a whole plant. Finally, PCR and Sanger sequencing are generally used for confirmation that the desired sequence edit has been successfully introduced into the selected plant. The selected plant is then examined to confirm that it exhibits the target trait of interest that was initially sought by introducing the genome modification.

Sandhya et al. 2020. J. Genet. Eng. Biotechnol. 18: 25. Published online 2020 Jul. 7. doi: 10.1186/s43141-020-00036-8, present tables showing which methods can be successfully applied to particular crops. For example, the following plants can all be successfully gene edited using PEG mediated delivery of CRISPR system components: Apple, Brassica oleracea, Brassica rapa, Citrullus lanatus, Glycine max, Grapevine, Oryza sativa, Petunia, Physcomitrella patens, Solanum lycopersicum, Triticum aestivum, and Zea mays. By way of further example, the following plants can all be successfully gene edited using particle bombardment mediated delivery of CRISPR system components: Glycine max, Hordeum vulgare, Oryza sativa, Triticum aestivum and Zea mays. By way of yet further example, the following plants can all be successfully gene edited using particle bombardment mediated delivery of CRISPR system components: Arabidopsis thaliana, Banana, Citrus sinensis, Cucumis sativum, Glycine max, Kiwi fruit, Lotus japonicus, Marchantia polymorpha, Medicago truncatula, Nicotiana benthamaina, Nicotiana tabacum, Oryza sativa, Populus, Salvia miltiorrhiza, Solanum lycopersicum, Solanum lycopersicum, Sorghum bicolor, Triticum aestivum, and Zea mays.

Activation of Polypeptides and their Homologs Through Targeted Genetic Modifications that Comprise the Mutation of uORFs:

Upstream ORFs (uORFs) comprise short sections of mRNAs that reside within the 5′ UTR or upstream region of a gene encoding a regulatory protein of interest (the coding sequence of which is often referred to as the main ORF or mORF). The uORF can be in frame or out-of-frame with the main coding sequence of the gene of interest. A substantial proportion of eukaryotic mRNAs contain uORFs in the 5′ leader sequence preceding the main functional protein-encoding ORF (Kochetov, 2008, BioEssays 30: 683-691). uORFs often encode short peptides which negatively regulate the activity of the regulatory proteins encoded by the genes of which they are upstream. Furthermore, uORFs sometimes initiate at a non-canonical codon (e.g., ACG rather than AUG) and the encoded peptide is often much less than 100 residues in length. These features make uORFs challenging to identify via automated bioinformatic searches, and experimentation is typically needed to confirm that a putative uORF functions as a negative regulator of a downstream coding sequence. For example, Laing et al., 2015. Plant Cell 27: 772-786; DOI: 10.1105/tpc.114.133777, removed a uORF that encodes a 60- to 65-residue peptide in the upstream region of GGP in lettuce, and showed this was sufficient to deliver a trait comprising increase levels of ascorbate. In fact, the peptides encoded by uORFs are very short indeed in some instances; for example, in humans, a functional peptide of only 6 amino acids was identified as being encoded by a uORF. Several plant uORFs have been shown to modulate mORF translation in response to the levels of various key metabolites within the cell (e.g., polyamines, sucrose, phosphocholine and ascorbate). A proposed function of several of the peptides encoded by these uORFs is to slow or stall the ribosomes and as a consequence limit translation of the downstream main ORF which encodes to regulatory protein (see Hellens et al., 2016. Trends Plant Sci., 21:317-328. dx.doi.org/10.1016/j.tplants.2015.11.005, and references therein).

Recently, it has been proposed that gene editing of uORFs may offer a general approach to activate crop genes to produce traits of interest in a highly targeted manner (Zhang and Voytas, 2019, Natl. Sci. Rev. 6: 391, doi.org/10.1093/nsr/nwy123). In particular, this can avoid many of the drawbacks associated with traditional approaches, which often involve large insertions of foreign DNA fragments, such as sequences of strong promoters, enhancers or engineered artificial transcription activators, in the genome. Indeed, many of the problems associated with genetic modifications that comprise transgene integrations, including lack of consumer acceptance of GM products, may be eliminated in a next generation of crop traits produced through knock-out of uORFs in regulator genes by targeted gene editing.

An increasing number of uORFs are being identified in the upstream regions of genes that encode transcriptional regulators and, in many cases, the uORF and/or its encoded short peptide appear to be controlled by a metabolic signal (van der Horst 2020. Plant Physiol. 182: 110-122, Published online 2019 Aug. 26. doi: 10.1104/pp.19.00940 and references therein). These include these the S1-group bZIPs, including the (HG1) bZIP transcription factor, which controls amino acid and sugar metabolism and which in turn has its activity regulated by sucrose. SAC51 (HG15) is bHLH transcription factor which is involved in xylem differentiation and regulated in response to thermospennine. Another example is the HsfB1/TBF1 (HG18) HSF transcription factor which is involved in heat tolerance and growth-to-defense transition which is regulated by galactinol.

A further example of transcription factor regulation by uORFs concerns a group of uORF-containing genes identified in the AUXIN RESPONSE FACTOR transcription factor family (Hellens 2016. Trend Plant Sci. 21:317-328; Schepetilnikov, M. et al. 2013, EMBO J. 32: 1087-1102; Nishimura, T. et al., 2005. Plant Cell 17: 2940-2953; Zhou, F. et al., 2010. BMC Plant Biol. 10: 193).

uORFs have also been identified as important in regulating the activity of transcription factors that control the light response, including transcription factors from the bZIP family (Kurihara et al., 2018. Proc. Natl. Acad. Sci. 115:7831-7836).

It should be noted that an additional confirmation/selection step is required to select one of more cells from the edited population that contain the desired change. These cells then can be regenerated into a population of cells, a tissue, organ or whole plant or plant population, which optionally, can be further screened to select plants which display the desired trait that is produced by the gene editing of a uORF sequence.

Traits that May be Modified

Trait modifications of particular interest include those to seed (such as embryo or endosperm), fruit, root, flower, leaf, stem, shoot, seedling or the like, including: enhanced tolerance to environmental conditions including freezing, chilling, heat, drought, water saturation, radiation and ozone; improved tolerance to microbial, fungal or viral diseases; improved tolerance to pest infestations, including insects, nematodes, mollicutes, parasitic higher plants (e.g. witchweed) or the like; decreased herbicide sensitivity; improved tolerance of heavy metals or enhanced ability to take up heavy metals; improved growth under poor photoconditions (e.g., low light and/or short day length), or changes in expression levels of genes of interest. Other phenotype that can be modified relate to the production of plant metabolites, such as variations in the production of taxol, tocopherol, tocotrienol, sterols, phytosterols, vitamins, wax monomers, anti-oxidants, amino acids, lignins, cellulose, tannins, prenyl lipids (such as chlorophylls and carotenoids), glucosinolates, and terpenoids, enhanced or compositionally altered protein or oil production (especially in seeds), or modified sugar (insoluble or soluble) and/or starch composition. Physical plant characteristics that can be modified include cell development (such as the number of trichomes), fruit and seed size and number, yields of plant parts such as stems, leaves, inflorescences, and roots, the stability of the seeds during storage, characteristics of the seed pod (e.g., susceptibility to shattering), root hair length and quantity, internode distances, or the quality of seed coat. Plant growth characteristics that can be modified include growth rate, germination rate of seeds, vigor of plants and seedlings, leaf and flower senescence, male sterility, apomixis, flowering time, flower abscission, rate of nitrogen uptake, osmotic sensitivity to soluble sugar concentrations, biomass or transpiration characteristics, as well as plant architecture characteristics such as apical dominance, branching patterns, number of organs, organ identity, organ shape or size.

### EXAMPLES

The invention, now being generally described, will be more readily understood by reference to the following examples, which are included merely for purposes of illustration of certain aspects and embodiments of the present invention and are not intended to limit the invention. It will be recognized by one of skill in the art that a genetic modification that is associated with a particular first trait may also be associated with at least one other, unrelated and inherent second trait which was not predicted by the first trait.

### Example 1

### Identifying the Presence of an Upstream Open Reading Frame.

1A. A method of identifying the presence of an upstream open reading frame (uORF) through application of an algorithm to ribosome profiling data, wherein the algorithm identifies the presence of the uORF based on the existence of ribosome enrichment in the interval from one stop codon to the next stop codon within the same open reading frame.
1B. The method of Statement 1A, wherein the identified uORF, or a uORF upstream of a main ORF that encodes a polypeptide that is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to a polypeptide encoded by a main ORF that is operably linked to the identified uORF, is mutated in a cell, and said mutation results in increased translation of the main ORF that is operably linked to the uORF.
1C. The method of Statement 1B, wherein the uORF is mutated by introducing at least one gene edit in the uORF.

### Example 2

Reducing the Function of uORFs in Plants
2A. The method of Statement 1A or Statement 1B, wherein the uORF is mutated n a plant, and said loss or reduction of function of the uORF results in increased translation of a polypeptide-encoding polynucleotide that is operably linked to the uORF.
2B. The method of Statement 2A wherein the polynucleotide encodes a polypeptide the expression of which confers cell death, inhibition of cell division, or an Improved Trait selected from the group consisting of:
   a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level.
2C. The method of Statement 2B, wherein the uORF regulates translation of the polynucleotide and derepression of said translation results in a toxic effect or cell death in the plant.
2D. The method of Statement 2B, wherein the plant is a weed or other undesirable plant.
2E. The method of Statement 2B, wherein the uORF regulates translation of the polynucleotide and the increased translation results in delayed flowering time or bolting in the plant as compared to a reference or control plant of the same species.
2F. The method of Statement 2B, wherein the uORF regulates translation of the polynucleotide and the increased translation results in earlier flowering time in the plant as compared to a reference or control plant of the same species.
2G. The method of any of Statements 2A-2F, wherein the plant is a crop plant, a fruit crop, a grain crop, a forage crop, a forest crop, an energy crop, a turf plant, a weed plant, a woody plant, a monocot plant, a dicot plant, an alga, a grass, an ornamental plant, a leafy plant, lettuce, a salad green, a pine, a eucalyptus, tomato, alfalfa, soybean, clover, carrot, celery, parsnip, cabbage, radish, rapeseed, broccoli, melon, cucumber, wheat, corn, cotton, rice, barley, millet, rye, potato, tomato, tobacco, sugar beet, sugar cane, Miscanthus, energy cane, bamboo, switchgrass, miscane, jatropha, Bermuda grass, lentil, chickpea, a pea, a bean, pepper, strawberry, blackberry, raspberry, blueberry, banana, pineapple, a Citrus, a nut crop, Hevea, oil palm, a coffee plant, a cocoa plant, a tea plant, or a plant that is a member of the family Solanaceae, Leguminosae, Umbelliferae, Curcurbitaceae, Gramineae, or Cruciferae.
2H. A method for increasing the yield, size, grain yield, seed yield, or biomass of a plant, wherein the plant is produced by any of the methods of Statements 2A-2G.
2I. A plant produced by the method of any of Statements 2A-2H.
2J. A plant or plant cell comprising:
   an introduced targeted genetic modification at a native genomic locus that comprises a mutation in a uORF, wherein the native genomic locus comprises an operably linked main ORF, downstream of the uORF, wherein the main ORF encodes a polypeptide with regulatory activity comprising an amino acid sequence with a percentage identity to a polypeptide selected from the group consisting of SEQ ID NO: 3999-5227.
   wherein the percentage identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100%; and
   the targeted genetic modification to the uORF increases expression level and/or activity of the encoded polypeptide with regulatory activity.
2K. A crop, turf, weed, or ornamental plant containing an introduced targeted genetic modification comprising a non-native allele that further comprises a mutation within a uORF that is operably linked to a polynucleotide comprising a main ORF that encodes a polypeptide with cellular regulatory activity that has an amino acid sequence identity to a sequence selected from the group consisting of SEQ ID NO: 3999-5227; and
   wherein said genetically modified plant exhibits an Improved Trait selected from the group consisting of:
   a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, increased cell death, increased leaf senescence, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level;
   as compared to a reference or control plant of the same species that lacks the non-native allele;
   and the amino acid sequence identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100%.
2L. The genetically modified crop, turf, weed, or ornamental plant of Statement 2K, wherein the uORF is targeted based on the final nucleotide of the uORF stop codon residing at a location between −1 and −1500 nucleotides upstream of the start codon of the polynucleotide that encodes the polypeptide with regulatory activity.
2M. The genetically modified crop plant of Statement 2K, wherein introduction of the targeted genetic modification into the plant does not result in negative effects on plant yield, size, organ shape or vigor and produces the modified crop plant that shows an Improved Trait selected from the group consisting of: a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level;
   when the genetically modified plant is grown under glasshouse or field conditions, as compared to the control or reference plant.
2N. The genetically modified plant of Statement 2K, wherein the non-native allele is selected and/or produced by a method selected from the group consisting of:
   DNA marker assisted breeding;
   deletion, insertion and/or substitution of one or more nucleotides;
   site-specific mutagenesis;
   chemical mutagenesis;
   targeting induced local lesions in genomes (TILLING); and
   a gene editing technique;
   wherein the gene editing technique includes a transcription activator-like effector nuclease (TALEN) or zinc finger nuclease (ZFN) based method, or a gene editing using a CRISPR-Cas endonuclease technique that uses a nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, or a gene editing technique that uses base editing deaminases, engineered site-specific meganucleases, an Argonaute related protein, or a CreI related endonuclease
2O. The genetically modified plant of Statement 2K, wherein the uORF comprises any of SEQ ID NO: 5156-5227.
2P. A method of producing an Improved Trait selected from the group consisting of:
   a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level in a crop plant comprising introducing a targeted genetic modification into the genome of said crop plant which creates a non-native allele of a gene which further comprises a mutation in a uORF that is operably linked to a polynucleotide that encodes a polypeptide with cellular regulatory activity that has an amino acid sequence with a percentage identity to a polypeptide selected from the group consisting of SEQ ID NO: 3999-5227;
   selecting a plant of the crop plant and wherein the selected plant contains the non-native allele and exhibits the Improved Trait compared to a reference or control plant of the same species that lacks the non-native allele;
   wherein the targeted genetic modification modulates the expression level and/or activity of the encoded polypeptide with transcriptional regulatory activity; and
   the percentage identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100%.
2Q. The genetically modified crop plant of Statement 2P, wherein the non-native allele is selected and/or produced
by a method selected from the group consisting of:
   DNA marker assisted breeding;
   deletion, insertion and/or substitution of one or more nucleotides;
   site-specific mutagenesis;
   chemical mutagenesis;
   targeting induced local lesions in genomes (TILLING); and
   a gene editing technique;
   wherein the gene editing technique includes a transcription activator-like effector nuclease (TALEN) or zinc finger nuclease (ZFN) based method, or a gene editing using a CRISPR-Cas endonuclease technique that uses a nuclease selected from Cas nuclease, Cas9 nuclease, CasX nuclease, CasY nuclease, a Cpf1 nuclease, a C2c1 nuclease, a C2c2 nuclease (Cas13a nuclease), or a C2c3 nuclease, NgAgo nuclease, or a gene editing technique that uses base editing deaminases, engineered site-specific meganucleases, an Argonaute related protein, or a CreI related endonuclease.
2R. The genetically modified crop plant of Statement 2P, wherein the introduction of the targeted genetic modification into the plant does not result in negative effects on plant size, organ shape or vigor and produces a modified plant that shows an Improved Trait selected from the group consisting of:
   a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level;
   when the modified plant is grown under glasshouse or field conditions, as compared to a control or reference plant that does not harbor the targeted genetic modification.
2S. The method of Statement 2P, wherein the location of the uORF is first identified by executing a computational algorithm that is applied to ribosome profiling data whereby the algorithm identifies the presence of the uORF in the polynucleotide that encodes the polypeptide, or in a polynucleotide that encodes a homolog with sequence similarity to the polypeptide, based on the existence of ribosome enrichment in the interval from one stop codon to the next stop codon within the same open reading frame.
2T. A process of killing the cells of plant, comprising contacting parts of said plant with a preparation of nanoparticles, or a suspension containing cells of an Agrobacterium strain, containing a nucleic acid construct comprising a gene editing system which expresses in the cells of said plant a guide RNA which introduces a mutation in a uORF that is upstream of a main ORF in the genome of said plant wherein said main ORF encodes a necrosis-inducing polypeptide that triggers death of the cells of the plant.
2U. The method of Statement 2T, wherein the plant is a weed.
2V. The method of Statement 2U, wherein the weed is selected from the list: Arabidopsis, Tall Waterhemp (Amaranthus tuberculatus), Johnson grass (Sorghum halepense), wild oat (Avena fatua), velvetleaf (Abutilon theophrasti), pigweed (Amaranthus palmeri), redroot pigweed (Amaranthus retroflexus) Poison Sumac (Toxicodendron Vernix), Japanese Knot Weed (Polygonum cuspidatum), Crabgrass (Digitaria), Dandelion (Leontodon taraxacum), Plantain (Plantago major), Common Ragweed (Ambrosia artemisiifolia), Giant Ragweed (Ambrosia trifida), Hedge Bindweed (Convolvus arvensis), Ground Ivy (Glechoma hederaceae), Purslane (Portulaca olearacea), Stinging Nettle (Urtica dioica), Curly Dock (Rumex crispus), Wild Madder (Galium mollugo), Clover Leaf (Trifolium species)
2W. The method of Statement 2T, wherein the gene editing system is a CRISPR-CAS system
2X. The method of Statement 2T, wherein the nucleic acid construct comprises a DNA sequence that encodes a CAS enzyme.
2Y. The method of Statement 2T wherein the main ORF encodes a polypeptide that comprises SEQ ID NO: 5152, 5153, 5154 or 5155 (AT4G36900, AT2G23340, AT5G67190, or AT3G50260) or a homolog with sequence similarity to SEQ ID NO: 5152, 5153, 5154 or 5155.
2Z. The method of Statement 2T, wherein the main ORF encodes a polypeptide that has at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to any of SEQ ID NO: 5152, 5153, 5154 or 5155 (AT4G36900, AT2G23340, AT5G67190, or AT3G50260).
2AA. The method of Statement 2T, wherein the uORF comprises any of SEQ ID NO: 5211-5227 inclusive.
2AB. An herbicidal composition comprising a preparation of nanoparticles, or a suspension containing cells of an Agrobacterium strain, containing a nucleic acid construct comprising a gene editing system which expresses in the cells of a target weed a guide RNA which introduces a mutation in a uORF that is upstream of a main ORF in the genome of said weed wherein said main ORF encodes a necrosis inducing polypeptide that triggers death of the cells of the plant.
2AC. The composition of Statement 2AB, wherein the gene editing system is a CRISPR-CAS system
2AD. The composition of Statement 2AB, wherein the nucleic acid construct comprises a DNA sequence that encodes a CAS enzyme.
2AE. The composition of Statement 2AB, wherein the main ORF encodes a polypeptide that comprises SEQ ID NO: 5152, 5153, 5154 or 5155 (AT4G36900, AT2G23340, AT5G67190, or AT3G50260) or a homolog of SEQ ID NO: 5152, 5153, 5154 or 5155.
2AF. The composition of Statement 2AB, wherein the main ORF encodes a polypeptide that has 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to any of SEQ ID NO: 5152, 5153, 5154 or 5155 (AT4G36900, AT2G23340, AT5G67190, or AT3G50260).
2AG. The composition of Statement 2AB, wherein the uORF comprises any of SEQ ID NO: 5211-5227 inclusive.

### Example 3

Genetic Modification of Microorganisms and the Use of uORF Mutations to Boost Production of Target Molecules and/or Enzymes in Cells Cultured Through Fermentation.
3A. A genetically modified cell comprising a non-naturally occurring polynucleotide that has been produced by gene editing, wherein the non-naturally occurring polynucleotide encodes a polypeptide that results in the production of an increased level of a target molecule or enzyme as compared to a control microorganism that does not comprise the non-naturally polynucleotide, and wherein non-naturally occurring polynucleotide contains a mutation in a uORF that resides in the same transcript as a main ORF that encodes the polypeptide.
3B. The genetically modified cell of Statement 3A, wherein the uORF is first identified through application of an algorithm to ribosome profiling data whereby the algorithm identifies the presence of the uORF in the polynucleotide that encodes the polypeptide, or in a polynucleotide that encodes a homolog of the polypeptide, based on the existence of ribosome enrichment in the interval from one stop codon to the next stop codon within the same open reading frame;
   wherein the homolog is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% to the polypeptide.
3B. The genetically modified cell of Statement 3A wherein the target molecule or enzyme is used for an application selected from the following: degradation of a pollutant, plastic degradation, oil degradation, use in laundry detergent; use as scent, use as flavoring, use as a pigment, use as a material, food processing, wood processing, antibiosis, treatment of cancer, treatment of diabetes, treatment of heart disease, treatment of hypertension, treatment of obesity, treatment of arthritis, treatment of degenerative disease, use as a psychoactive substance, treatment of anxiety, treatment of a behavioral disorder, use as a food supplement, use as a digestive aid, use as an herbicide, use as an insecticide, use as a fungicide, use as a rodenticide, use as a bactericide, use as a nematicide, use as an algicide or use as an anti-viral agent.
3D. The genetically modified cell of Statement 3A wherein the target molecule or enzyme is produced in a fermentation process.
3F. The genetically modified cell of Statement 3A wherein the cell is a fungal cell, a bacterial cell, a mammalian cell, or a plant cell.
3G. The genetically modified cell of statement 3A wherein the molecule is encoded by a biosynthetic gene cluster (BGC) and wherein the uORF reside in the 5′ region of a transcript (mRNA) that encodes for a transcriptional regulator protein that regulates expression of genes in the biosynthetic gene cluster.

### Example 4

### Control of Cancerous Cells

Typically, the practitioner commences by selecting a published ribosome profiling dataset, or experimentally generating a new ribosome profiling dataset by performing ribosome “pull-downs” on mRNA samples purified from cancerous tissue or a cancer cell line. RNASeq is performed whereby the pulled down RNA is reverse transcribed and subjected to deep sequencing (e.g., 50× coverage using the Illumina system). An algorithm of the type detailed herein is the run on the sequence data to identify uORFs in stop-stop intervals. A gene that contains a uORF, and where the main ORF of which gene encodes a cell death promoting protein or cell cycle inhibitor, is then selected for the process of killing the cancer cells.

In a more specific embodiment of the invention, a tumor or cancer cell line is subject to ribosome profiling and the data are analyzed by application of the algorithm detailed herein to identify loci that are subject to uORF regulation. A locus is selected that has a uORF and an operably linked main ORF that encodes a polypeptide that has a function in tumor suppression, cell death, or inhibition of cell division. A gene editing construct that encodes a guide RNA with identity to a uORF at the selected locus, which has is designed to knock-out or mutate the uORF. The gene editing construct is then delivered to a tumor or cancer cells in vivo by means of a delivery system such as viral vector. The disruption of the uORF in the cancer cells or cells of the tumor results in increased translation of the main ORF which leads to control of the tumor or cancer cells.

A practitioner may apply the methods herein to identify oncogenes that are uORF controlled by comparing ribosome pull down data from a cancerous tissue or cell line compared to ribosome pull down data from a control tissue. Typically, the practitioner commences by selecting a published ribosome profiling dataset from a cancer cell line, or experimentally generating a new ribosome profiling dataset by performing ribosome “pull-downs” on mRNA samples purified from cancerous tissue or cells, along with samples from control non-cancerous cells. RNASeq is performed whereby the pulled down RNA is reverse transcribed and subjected to deep sequencing (e.g. 50× coverage using the Illumina system). An algorithm of the type detailed herein is the run on the sequence data to identify uORFs. A gene that contains an identified upstream uORF in the control sample, where the uORF contains a mutation in the sample from the cancerous tissue may be considered a candidate oncogene. Additional support that an identified gene is a likely oncogene may be obtained by performing a BLAST of the product of the main ORF against public databases; if the main ORF product shows homology to known cell cycle regulators, it is a strong candidate oncogene that may be contributing to cancerous nature of the cells in which it is active. Conversely, if a novel uORF is apparent upstream of a main ORF in the cancerous sample, and appears to have been generated by mutation, by comparison to the control sample, the created uORF may be suppressing an anti-cancer gene.
4A. A process of controlling cancerous cells or cells of a tumor, comprising contacting the cancerous cells or cells of the tumor with a delivery vector containing a nucleic acid construct comprising a gene editing system which expresses in the cells a guide RNA which introduces a mutation in a uORF that is upstream of a main ORF in the genome of said cells wherein said main ORF encodes polypeptide that triggers death or inhibits cell division of the cancerous cells or cells of the tumor.
4B. The process of Statement 4A, wherein the uORF is first identified through application of an algorithm to ribosome profiling data whereby the algorithm identifies the presence of the uORF in the main ORF that encodes the polypeptide or in a main ORF that encodes a homolog of the polypeptide based on the existence of ribosome enrichment in the interval from one stop codon to the next stop codon within the same open reading frame.
4B. The process of Statement 4A wherein the main ORF comprises a cancer suppressor gene.
4D. The process of Statement 4A wherein the main ORF encodes a polypeptide that inhibits cell division.
4E. The process of Statement 4A wherein the delivery vector is a viral vector.
4F. As discussed herein, there are instances where a uORF residing upstream of a main ORF encodes a short peptide or uPEP which acts to inhibit, either directly or indirectly, the activity of the main ORF. In instances where the uORF is upstream of a main ORF that promotes cell division, or is acting through some other mechanism to promote a cancerous state, the uPEP may be formulated and delivered as a drug (e.g., orally or intravenously) to inhibit the activity of the cancer causing gene and thereby control the cancerous cells.
   In such instances, the uPEP may be synthesized either through fermentation (e.g., in yeast or E. coli) or synthesized artificially, formulated (e.g., to promote stabilization and/or cellular entry) and delivered to a patient either orally or intravenously to control the cancer.

### Example 5

### Control of Eukaryotic Pests and Pathogens

The examples below have the advantage of using an exogenously applied nucleic acid that is specific to a target pest or pathogen, which is superior to use of chemical agent, which often act non-specifically in a broadly toxic maimer and have detrimental effects on non-target organisms.
Typically, the practitioner commences by selecting a published ribosome profiling dataset, or experimentally generating a new ribosome profiling dataset by performing ribosome “pull-downs” on mRNA samples purified from tissue or cells of the target pathogen. RNASeq is performed whereby the pulled down RNA is reverse transcribed and subjected to deep sequencing (e.g., 50× coverage using the Illumina system). An algorithm of the type detailed herein is the run on the sequence data to identify uORFs. A gene that contains a uORF, and where the main ORF of which gene encodes a cell death promoting protein or cell cycle inhibitor, is then selected for the process of controlling the pest or pathogen.
5A. A process of controlling a eukaryotic pest or pathogen, comprising contacting cells of the eukaryotic pest or pathogen with a delivery vector containing a nucleic acid construct comprising a gene editing system which expresses in the cells a guide RNA which introduces a mutation in a uORF that is upstream of a main ORF in the genome of said cells wherein said main ORF encodes polypeptide that triggers death or inhibits cell division of the cells of the eukaryotic pathogen.
5B. The process of Statement 5A, wherein the uORF is first identified through application of an algorithm to ribosome profiling data whereby the algorithm identifies the presence of the uORF in the main ORF that encodes the polypeptide or in a main ORF that encodes a gene with sequence similarity of the polypeptide based on the existence of ribosome enrichment in the interval from one stop codon to the next stop codon within the same open reading frame;
   wherein the gene with sequence similarity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to the polypeptide.
5C. The process of Statement 5A, wherein the main ORF encodes a polypeptide that inhibits cell division.
5D. The process of Statement 5A, wherein the delivery vector is a viral vector or an antibody.
5E. The process of Statement 5A, wherein the pathogen is a fungus.
5F. The process of Statement 5A, wherein the pathogen is a protozoan.
5G. The process of Statement 5A, wherein the pathogen is a malarial cell.
5H. The process of Statement 5A, wherein the pathogen is a parasitic worm.
5I. In instances where an essential gene (aka “lethal” gene) from a pest or pathogen is essential for the pest or pathogen to develop or complete its lifecycle and the essential gene possesses a uORF in its transcript, upstream of the main ORF, the uPEP encoded by the uORF may provide an effective agent for control of the pest or pathogen by its exogenous application as a pesticide. Ideally, an essential gene will be selected which is specific to the particular type of pest and which is either not present or is non-essential in mammals. An example would be genes involved in chitin biosynthesis for example, if a practitioner is seeking to control a fungus or an insect. In the case of an herbicide, a uORF is sought in a plant specific essential gene, such as a critical gene involved in amino acid synthesis, plant hormone production, meristem development, or photosynthesis.
   In the above instances, the practitioner uses the uORF sequence to heterologously produce the encoded uPEP in a fermentation system (e.g., E. coli, yeast, or a cell line) and the resulting peptide is formulated to stabilize it and/or promote cellular entry and is then exogenously applied to the pest as a pesticidal agent.

### Example 6

Identifying uORFs in Heterologous Genes
Sequence similarity between mORFs of different species can be used to identify uORFs in heterologous genes. AT1G01060.1 (SEQ ID NO: 4000; LHY) encodes a MYB-related putative transcription factor involved in circadian rhythm and was identified as a new uORF-containing gene candidate. The protein sequence of LHY from Arabidopsis was used to identify the LHY orthologs in Brassica oleraceae. The AT1G01060.1 sequence was then used in a sequence homology alignment search of the genome Brassica oleracea using BLAST (tblastn) at genomevolution.org/coge/CoGeBlast.pl as well as in a range of other species Results are shown in Table 2.

TABLE 3
Putative orthologs of LHY/CCA1 from sugar beet (Beta vulgaris), Eucalyptus (Eucalyptus grandis),
barrel medic (Medicago truncatula), brassica spp. (Brassica oleracea), and orthologs
of A5-DREB from pigweed (Amaranthus hybridus) through BLAST analysis

| | Chr | Position | HSP# | E-value | Quality | Closest Genomic Feature |
|---|---|---|---|---|---|---|
| Beta vulgaris (Adrew Funk | 1_EL10.1 | 2029057 | 1 | 2.00E−38 | 21.80% | E10Ac1g00171.1:8 |
| FASTA vEL10_1.0) | | | | | | |
| Beta vulgaris (Adrew Funk | 4_EL10.1 | 36341401 | 2 | 2.00E−19 | 7.40% | none |
| FASTA vEL10_1.0) | | | | | | |
| Beta vulgaris (Adrew Funk | 1_EL10.1 | 2027891 | 3 | 2.00E−38 | 9.30% | E10Ac1g00170.1:13 |
| FASTA vEL10_1.0) | | | | | | |
| Beta vulgaris (Adrew Funk | 1_EL10.1 | 2040547 | 4 | 7.00E−15 | 5.30% | E10Ac1g00171.1:4 |
| FASTA vEL10_1.0) | | | | | | |
| Beta vulgaris (Adrew Funk | 7_EL10.1 | 29147606 | 5 | 1.00E−06 | 4.00% | none |
| FASTA vEL10_1.0) | | | | | | |
| Eucalyptus grandis BRASUZ1 | 4 | 40431588 | 1 | 7.00E−47 | 0.329 | Eucgr.D02651.2.v2.0 |
| Eucalyptus grandis BRASUZ1 | 6 | 41227391 | 2 | 5.00E−19 | 0.048 | Eucgr.F02906.1.v2.0 |
| Eucalyptus grandis BRASUZ1 | 4 | 40426987 | 3 | 1.00E−12 | 0.053 | Eucgr.D02651.1.v2.0 |
| Eucalyptus grandis BRASUZ1 | 11 | 30919289 | 4 | 7.00E−19 | 0.043 | Eucgr.K02302.1.v2.0 |
| Eucalyptus grandis BRASUZ1 | 1 | 42597650 | 5 | 0.000004 | 0.037 | Eucgr.A02733.1.v2.0 |
| Eucalyptus grandis BRASUZ1 | 11 | 30919483 | 6 | 7.00E−19 | 0.031 | Eucgr.K02302.1.v2.0 |
| Eucalyptus grandis BRASUZ1 | 6 | 41227587 | 7 | 5.00E−19 | 0.029 | Eucgr.F02906.1.v2.0 |
| Eucalyptus grandis BRASUZ1 | 4 | 40426881 | 8 | 1.00E−12 | 0.012 | Eucgr.D02651.1.v2.0 |
| Medicago truncatula (barrel | 7 | 49122314 | 1 | 2.00E−42 | 32.60% | Medtr7g118330.1.JCVIMt4.0v1 |
| medic) | | | | | | |
| Medicago truncatula (barrel | 6 | 28640709 | 2 | 3.00E−18 | 7.00% | Medtr6g477860.2.JCVIMt4.0v1 |
| medic) | | | | | | |
| Medicago truncatula (barrel | 7 | 49115772 | 3 | 2.00E−21 | 5.40% | Medtr7g118330.1.JCVIMt4.0v1 |
| medic) | | | | | | |
| Medicago truncatula (barrel | 5 | 32830655 | 4 | 1.00E−12 | 4.50% | Medtr5g076960.2.JCVIMt4.0v1 |
| medic) | | | | | | |
| Medicago truncatula (barrel | 3 | 29065130 | 5 | 1.00E−18 | 4.80% | Medtr3g064500.1.JCVIMt4.0v1 |
| medic) | | | | | | |
| Medicago truncatula (barrel | 7 | 49115962 | 6 | 2.00E−21 | 3.10% | Medtr7g118330.1.JCVIMt4.0v1 |
| medic) | | | | | | |
| Medicago truncatula (barrel | 3 | 29065360 | 7 | 1.00E−18 | 2.80% | Medtr3g064500.1.JCVIMt4.0v1 |
| medic) | | | | | | |
| Brassica oleracea (Ensembl | C5 | 409337 | 1 | 2.00E−126 | 40.30% | CDS:Bo5g002760.1 |
| Plants Masked by source v2.1) | | | | | | |
| Brassica oleracea (Ensembl | C5 | 410562 | 3 | 2.00E−126 | 14.70% | CDS:Bo5g002760.1 |
| Plants Masked by source v2.1) | | | | | | |
| Brassica oleracea (Ensembl | C3 | 37760 | 4 | 5.00E−71 | 26.00% | CDS:Bo3g001090.1 |
| Plants Masked by source v2.1) | | | | | | |
| Brassica oleracea (Ensembl | C3 | 38526 | 6 | 5.00E−71 | 13.30% | CDS:Bo3g001090.1 |
| Plants Masked by source v2.1) | | | | | | |
| Brassica oleracea (Ensembl | C3 | 4200012 | 8 | 2.00E−21 | 6.70% | CDS:Bo3g012470.1 |
| Plants Masked by source v2.1) | | | | | | |
| Amaranthus hybridus subsp. | tig00000569_arrow | 426296 | 1 | 3.00E−37 | 39.80% | Ah.00g189710.m01-v1.0.a1 |
| Hybridus | | | | | | |
| Amaranthus hybridus subsp. | tig00000007_arrow | 3229227 | 2 | 2.00E−35 | 38.70% | Ah.00g024560.m01-v1.0.a1 |
| Hybridus | | | | | | |
| Amaranthus hybridus subsp. | tig00068853_arrow | 5143989 | 3 | 1.00E−20 | 23.00% | Ah.00g217670.m01-v1.0.a1 |
| Hybridus | | | | | | |
| Amaranthus hybridus subsp. | tig00000084_arrow | 2199749 | 4 | 1.00E−19 | 20.90% | Ah.00g105020.m01-v1.0.a1 |
| Hybridus | | | | | | |
| Amaranthus hybridus subsp. | tig00000048_arrow | 3920876 | 5 | 1.00E−19 | 20.90% | Ah.00g088580.m01-v1.0.a1 |
| Hybridus | | | | | | |
| Amaranthus hybridus subsp. | tig00000163_arrow | 935318 | 6 | 3.00E−19 | 19.90% | Ah.00g139080.m01-v1.0.a1 |
| Hybridus | | | | | | |
| Amaranthus hybridus subsp. | tig00000032_arrow | 6069826 | 7 | 6.00E−19 | 20.90% | Ah.00g064960.m01-v1.0.a1 |
| Hybridus | | | | | | |
| Amaranthus hybridus subsp. | tig00000004_arrow | 9106529 | 8 | 7.00E−19 | 19.90% | Ah.00g022310.m01-v1.0.a1 |
| Hybridus | | | | | | |

### Example 7

Identification of uORFs in Cell Death Inducing Genes from an Example Weed

The protein sequence of AT4G36900 from Arabidopsis was used to identify the orthologous gene from pigweed (Amaranthus hybridus) as shown in the bottom eight rows of the above Table 3. At4g36900.1 encodes a member of the DREB subfamily A-5 of ERF/AP2 transcription factor family (RAP2.10), and was identified in a high throughput analysis as a uORF-containing gene candidate.

The following sequence was used in a sequence homology alignment search of the Amaranthus hybridus genome using BLAST at genomevolution.org/coge/CoGeBlast.pl

(AT4G36900, SEQ ID NO: 5152)
METATEVATVVSTPAVTVAAVATRKRDKPYKGIRMRKWGKWVAEIREPN
KRSRIWLGSYSTPEAAARAYDTAVFYLRGPSARLNFPELLAGVTVTGGG
GGGVNGGGDMSAAYIRRKAAEVGAQVDALEAAGAGGNRHHHHHQHQRGN
HDYVDNHSDYRINDDLMECSSKEGFKRCNGSLERVDLNKLPDPETSDDD.

The output of this sequence search identified the closest gene with sequence homology to AT4G36900 (SEQ ID NO: 5152) as Ah.03g145670.m01-v1.1.a1

Inspection of this locus in the genome using Jbrows reveal the coding sequence and its adjacent upstream sequence that corresponds to the leader region.

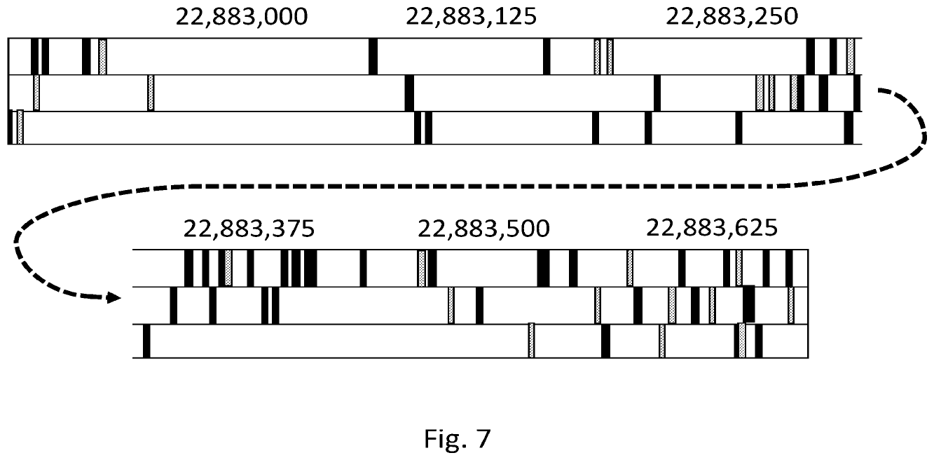

FIG. 7 shows Amaranthus hybridus subsp. hybridus (hybridus contigs scaffolded to hypochondriacus): polished genome contigs of Amaranthus hybridus scaffolded to pseudochromosomes of Amaranthus hypochondriacus with reveal finish (v1.0, id57429). Gray bars present putative AUG start codon is and regards represent stop codons for each of the three reading frames (noting the gene is in reverse order). In frame open reading frames that could potentially be uORF are defined by these stop-stop intervals. In this way, putative uORF of genes can be identified and tested as candidates for gene editing.

### Example 8

Identification of a Set of Genes Containing uORFs from Arabidopsis, and Subsequent Identification of the Corresponding Genes in Target Crops

To reduce the invention detailed herein to practice, codes 1 to 3 were applied to ribosome profiling data from Arabidopsis to identify a set of loci, identified by Arabidopsis genome identifiers, which are uORF-containing candidate genes (SEQ ID NO: 1 to 5155). These loci correspond to the Arabidopsis gene identifiers in the <223> comment line of each of the SEQ ID NO: 1 through 5155. Note that in some cases, a locus is represented by different gene models or multiple different gene models in the Sequence Listing, and in other instances a given model for a locus has multiple predicted uORFs. Because the presence of uORFs is often evolutionarily conserved, these data provide a road map for generation of Improved Traits through gene editing of uORFs in orthologous loci in a target crop. In one embodiment of the invention, the sequence of a polypeptide encoded by a locus which is known to produce an Improved Trait of interest when the polypeptide is present at an increased level, is compared against a set of proteins from a target crop by application of BLAST or alignment analysis. A crop locus is identified that comprises a main ORF that encodes a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide that was used for the comparison. The region of crop genomic DNA that is 1-1500 bp upstream of the start codon of the crop main ORF is then bioinformatically analyzed to identity stop-stop open reading frames upstream of the crop main ORF. A polynucleotide construct is designed to encode a guide RNA that introduces a mutation in the identified upstream open reading frame(s). The guide RNA is delivered to cells of the target crop by means of a gene editing system and crop plants are regenerated and selected that exhibit the Improved Trait of interest.

### Example 9

Detection of uORFs in Example Selected Regulatory Genes in Different Plant Species

In this example, uORFs were detected in target crops and other plants including sugar beet, Eucalyptus, broccoli, and Amaranthus. Candidate genes were identified through a homology search to genes of interest from Arabidopsis. Candidate uORF sequences were then extracted upstream of the candidate gene.

The candidate uORFs are those with any start codon (sometimes a stop, sometimes the codon after a stop if there are multiple stop codons between this and the preceding uORF) that are over fifty nucleotides. These were extracted are shown in Table 4. Three examples for LATE ELONGATED HYPOCOTYL (LHY; encodes a MYB-related putative transcription factor involved in circadian rhythm) and one for a Dehydration Responsive Element Binding” transcription factor (DREB; involved in regulation of expression of many stress-inducible genes) are provided.

TABLE 4
uORFs detected in target plants

| | | |
|---|---|---|
| Potential uORF (reading | | |
| frame 1 to 3) | Sequence Length | SEQ ID NO: |

LHY search in Beta vulgaris (sugar beet) with AT1G01060

| | | |
|---|---|---|
| ORF 1 (frame 1) | >276 | 5156 |
| ORF 2 (frame 1) | 111 | 5157 |
| ORF 3 (frame 1) | 90 | 5158 |
| ORF 4 (frame 1) | 87 | 5159 |
| ORF 5 (frame 1) | 78 | 5160 |
| ORF 6 (frame 1) | 69 | 5161 |
| ORF 7 (frame 2) | >73 | 5162 |
| ORF 8 (frame 2) | 147 | 5163 |
| ORF 9 (frame 2) | 69 | 5164 |
| ORF 10 (frame 2) | 69 | 5165 |
| ORF 11 (frame 2) | 78 | 5166 |
| ORF 12 (frame 2) | 114 | 5167 |
| ORF 13 (frame 2) | 144 | 5168 |
| ORF 14 (frame 2) | 60 | 5169 |
| ORF 15 (frame 2) | >85 | 5170 |
| ORF 16 (frame 3) | >392 | 5171 |
| ORF 17 (frame 3) | 75 | 5172 |
| ORF 18 (frame 3) | 72 | 5173 |
| ORF 19 (frame 3) | 141 | 5174 |
| ORF 20 (frame 3) | 75 | 5175 |

| | | |
|---|---|---|
| Potential uORF | Sequence Length | SEQ ID NO: |

LHY search in Eucalyptus grandis with AT1G01060

| | | |
|---|---|---|
| ORF 1 (frame 1) | >135 | 5176 |
| ORF 2 (frame 1) | 132 | 5177 |
| ORF 3 (frame 1) | 201 | 5178 |
| ORF 4 (frame 1) | 63 | 5179 |
| ORF 5 (frame 1) | >166 | 5180 |
| ORF 6 (frame 2) | >280 | 5181 |
| ORF 7 (frame 2) | 144 | 5182 |
| ORF 8 (frame 2) | 156 | 5183 |
| ORF 9 (frame 2) | 90 | 5184 |
| ORF 10 (frame 3) | >143 | 5185 |
| ORF 11 (frame 3) | 306 | 5186 |
| ORF 12 (frame 3) | 201 | 5187 |
| ORF 13 (frame 3) | >62 | 5188 |

LHY search in Brassica oleracea (broccoli) with AT1G01060

| | | |
|---|---|---|
| ORF 1 (frame 1) | 162 | 5189 |
| ORF 2 (frame 1) | 51 | 5190 |
| ORF 3 (frame 1) | 72 | 5191 |
| ORF 4 (frame 1) | 69 | 5192 |
| ORF 5 (frame 1) | 87 | 5193 |
| ORF 6 (frame 1) | 78 | 5194 |
| ORF 7 (frame 1) | 63 | 5195 |
| ORF 8 (frame 1) | >69 | 5196 |
| ORF 9 (frame 2) | 84 | 5197 |
| ORF 10 (frame 2) | 105 | 5198 |
| ORF 11 (frame 2) | 51 | 5199 |
| ORF 12 (frame 2) | 84 | 5200 |
| ORF 13 (frame 2) | 108 | 5201 |
| ORF 14 (frame 2) | 129 | 5202 |
| ORF 15 (frame 3) | >53 | 5203 |
| ORF 16 (frame 3) | 108 | 5204 |
| ORF 17 (frame 3) | 51 | 5205 |
| ORF 18 (frame 3) | 54 | 5206 |
| ORF 19 (frame 3) | 63 | 5207 |
| ORF 20 (frame 3) | 75 | 5208 |
| ORF 21 (frame 3) | 108 | 5209 |
| ORF 22 (frame 3) | 165 | 5210 |

DREB/ERF/AP2 search in Amaranthus hybridus with At4g36900.1

| | | |
|---|---|---|
| ORF 1 (frame 1) | 60 | 5211 |
| ORF 2 (frame 1) | 60 | 5212 |
| ORF 3 (frame 1) | 63 | 5213 |
| ORF 4 (frame 1) | 141 | 5214 |
| ORF 5 (frame 1) | 90 | 5215 |
| ORF 6 (frame 1) | 150 | 5216 |
| ORF 7 (frame 2) | 72 | 5217 |
| ORF 8 (frame 2) | 243 | 5218 |
| ORF 9 (frame 2) | 60 | 5219 |
| ORF 10 (frame 2) | 75 | 5220 |
| ORF 11 (frame 2) | 87 | 5221 |
| ORF 12 (frame 2) | >200 | 5222 |
| ORF 13 (frame 3) | 87 | 5223 |
| ORF 14 (frame 3) | 108 | 5224 |
| ORF 15 (frame 3) | 78 | 5225 |
| ORF 16 (frame 3) | 129 | 5226 |
| ORF 17 (frame 3) | >193 | 5227 |

### Example 10

Delivery of Delayed Flowering, Enhanced Yield and/or Increased Biomass Related Traits by Targeting CCA1 Related Genes

In a further embodiment of the invention, a crop homolog of the Arabidopsis circadian clock regulation protein LATE ELONGATED HYPOCOTYL (LHY/AT1G01060) and CIRCADIAN CLOCK ASSOCIATED 1 (CCA1/AT2G46830), which are shown to be subject to uORF regulation herein, is upregulated through knock-out or mutation of an operably linked uORF by means of gene editing or TILLING. Specifically, a genetic modification is introduced to a uORF within the endogenous locus that contains a main ORF encoding a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to (AT1G01060.1, SEQ ID NO: 4000) or (AT2G46830.1; SEQ ID NO: 1678). Such a genome modification produces crop plants that show a yield increase and/or delayed flowering and/or increased vegetative mass when grown in a glasshouse, growth room or field. Under such conditions, crop plants containing the targeted introduced genetic modification exhibit at least a 2% yield increase, or at least a 3% yield increase, or at least a 4% yield increase, or at least a 6% yield increase, or at least an 8% yield increase, or at least 10% yield increase, or at least a 20% yield increase, or at least a 50% yield increase, compared to control plants not harboring the genetic modification. In a particular embodiment of this example, the genetically modified crop plant is a leafy green or a forage crop, or a crop where the vegetative portion of the plant comprises the desired crop. Sugar beet, for example, is a crop of the latter category, where a large vegetative storage organ is sought, and flowering is undesirable. In another embodiment of the invention, the genetically modified crop plant is a tree crop which shows delayed flowering, or never flowers prior to harvest. This is especially desirable in transgenic trees being grown for biomass, such as Eucalyptus and poplar.

### Example 11

### Induction of Flowering by Targeting FCA Related Genes

In a further embodiment of the invention, a crop homolog of the Arabidopsis flowering time regulator FCA (AT4G16280.2; SEQ ID NO: 4788), which is shown to be subject to uORF regulation herein, is upregulated through knock-out or mutation of operably linked uORFs by means of gene editing. Specifically, a genetic modification is introduced to a uORF within the endogenous locus that contains a main ORF that encodes a polypeptide that has, or a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to SEQ ID NO: 4788. Such a genome modification produces crop plants that show early flowering in a glasshouse, growth room or field. Under such conditions, crop plants containing the targeted introduced genetic modification exhibit floral structures at least 1 day earlier, 5 days earlier, 10 days earlier, 30 days earlier, 60 days earlier or 180 days earlier compared to control plants not harboring the genetic modification.

### Example 12

Method of Controlling a Weed by Identification and Targeting of uORFs from Cell Death-Inducing Genes in the Control Regions that Encode AP2 Family Transcription Factors.

In another embodiment of the invention, a weed plant is subject to ribosome profiling and the data are analyzed by application of the algorithm detailed herein to identify loci that are subject to uORF regulation. A locus is selected that has a uORF and an operably linked main ORF that encodes a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by either AT4G36900, AT2G23340, AT5G67190, or AT3G50260 (SEQ ID NO: 5152, 5153, 5154 or 5155, respectively. These proteins form a clade within the AP2 family of transcription factors. A gene editing construct is designed that encodes a guide RNA with identity to a uORF at the selected locus, which is designed to knock-out or mutate the uORF. The gene editing construct is then delivered to weeds by means of an Agrobacterium suspension coated on nanoparticles or via some other appropriate formulation. The disruption of the uORF in cells of the target weed results in increased translation of the homolog of AT4G36900, AT2G23340, AT5G67190, or AT3G50260 which leads to cell death and control of the target weed.

### Example 13

Increasing BRIX and/or Sugar Content by Targeting of uORFs from bZIP Family Transcription Factors

In another embodiment of the invention, a fruit or vegetable plant is chosen and a locus from the genome of that fruit or vegetable plant is selected that has a uORF and an operably linked main ORF that encodes a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by bZIP protein AT4G34590.1, SEQ ID NO: 4871). A gene editing construct that encodes a guide RNA with identity to a uORF at the selected locus, which has a base change designed to knock-out or mutate the uORF. The gene editing construct is then delivered to cells of the fruit or vegetable plant. Plants are then regenerated which carry a genetic modification whereby the uORF repression has been reduced or removed and the fruit or vegetable plants have increased sugar content or BRIX content, compared to control plants not harboring the genetic modification when grown in a glasshouse, growth room or field. In particular embodiment of this example, the plant is a member of the nightshade family such as tomato.

### Example 14

Increasing Cold Tolerance by Targeting of uORFs from Myb Family Transcription Factors

In another embodiment of the invention, a crop is chosen and a locus from the genome of that crop plant is selected that has a uORF and an operably linked main ORF that encodes a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by MYB protein AT1G74650 (SEQ ID NO: 4274). A gene editing construct that encodes a guide RNA with identity to a uORF at the selected locus, which has a base change designed to knock-out or mutate the uORF. The gene editing construct is then delivered to cells of the crop plant. Plants are then regenerated which carry a genetic modification whereby the uORF repression has been reduced or removed and the crop plants have increased cold tolerance, compared to control plants not harboring the genetic modification when grown in a glasshouse, growth room or field.

### Example 15

Increasing Nutritional Content or Pigmentation of Plant Tissue by Targeting uORFs in HB Family Transcription Factors that are Required for Flavonoid Production.

In another embodiment of the invention, a crop is chosen and a locus from the genome of that crop plant is selected that has a uORF and an operably linked main ORF that encodes a polypeptide that has, or a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by homeodomain protein ANTHOCYANINLESS2 AT4G00730 (SEQ ID NO: 4731). A gene editing construct that encodes a guide RNA with identity to a uORF at the selected locus, which is designed to knock-out or mutate the uORF. The gene editing construct is then delivered to cells of the crop plant. Plants are then regenerated which carry a genetic modification whereby the uORF repression has been reduced or removed and the crop plants have increased pigment levels, compared to control plants not harboring the genetic modification when grown in a glasshouse, growth room or field.

### Example 16

Increasing Yield, Vigor, Seedling Size, Drought Tolerance, Protein Content, and/or Tolerance to Abiotic Stresses by Targeting uORFs in NF-Y (Aka HAP or CAAT) Family Transcription Factors.

The NF-Y family of transcription factors have been shown to regulate a wide range of critical processes including improved seedling vigor, flowering time, nutritional content and/or stress tolerance through transgenic approaches, including overexpression of the native forms of the genes encoding these TFs (US Patent Plants with enhanced size and growth rate (Nelson et al. 2007, PNAS 104 (42) 16450-16455; Kumimoto et al. 2008, Planta 228, 709-723; U.S. Pat. Nos. 8,927,811; 10,640,781). This example provides a way to obtain the same or similar traits, without undesirable phenotypes such as morphological abnormalities, extreme alterations in flowering time and/or dwarfing, through the alternative means of gene editing the endogenous loci encoding the genes in crop plants, and/or through overexpression of NF-Y genes with modified or deleted uORFs in the 5′UTR regions of the overexpressed transcripts.

In an embodiment of the invention, a crop is chosen and a locus from the genome of that crop plant is selected that has a uORF operably linked main ORF that encodes a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by HAP2 protein AT5G12840 (SEQ ID NO: 4961). A gene editing construct is built that encodes a guide RNA with identity to a uORF at the selected locus, which is designed to knock-out or mutate the uORF. The gene editing construct is then delivered to cells of the crop plant. Plants are then regenerated which carry a genetic modification whereby the uORF repression has been reduced or removed and a plant is selected that has increased drought tolerance, increased seedling size, increased vigor, increased abiotic stress tolerance and/or increased yield, but lacks any substantive undesirable development phenotype, as compared to control plants not harboring the genetic modification when grown in a glasshouse, growth room or field.

In a further embodiment of the invention, the NF-YC transcription factor is a member of the NF-YC4 subclade orthologous to the Arabidopsis paralogs AT3G48590 and AT5G63470, which regulate beneficial traits including enhanced vigor, increased abiotic stress tolerance, increased nutrient content and increased tolerance to biotic stress including viruses, bacteria, fungi, aphids and nematodes (U.S. Pat. No. 10,640,781; Ling Li et al. PNAS Nov. 24, 2015 112 (47) 14734-14739; Mingsheng Qi et al. Plant Biotechnology Journal (2019) 17, pp. 252-263). A crop is chosen and a locus from the genome of that crop plant is selected that has a uORF operably linked to a main ORF that encodes a polypeptide that has, or has a region with, at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by AT3G48590 or AT5G63470. A gene editing construct is built that encodes a guide RNA with identity to a uORF at the selected locus, which is designed to knock out or mutate the uORF. The gene editing construct is then delivered to cells of the crop plant. Plants are then regenerated which carry a genetic modification whereby the uORF repression has been reduced or removed and a plant is selected that has an increased level of the polypeptide, abiotic stress tolerance, increased yield, increased vigor, increased calorific content, and/or increased nutritional content compared to control plants not harboring the genetic modification when grown in a glasshouse, growth room or field.

In a yet further embodiment of the invention, a uORF is mutated in the 5′ region comprising a genetic modification to the endogenous locus encoding a crop homolog of NF-YC4 transcription factors AT3G48590 and AT5G63470. Such a genome modification produces plants that show increased seedling vigor and/or improved abiotic stress tolerance and/or improved photosynthesis and/or increased protein levels in tissues, when grown under glasshouse conditions, field conditions and/or conditions of dehydration stress, heat stress and/or salt stress. Under such conditions, the crop plant containing this targeted introduced genetic modification comprises a non-native allele of a gene with a mutated uORF upstream of a main ORF that encodes a polypeptide with at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to the polypeptide encoded by AT3G48590 or AT5G63470. A crop plant containing the aforementioned targeted genetic mutation is then selected which exhibits at least a 2% protein content increase, or at least a 3% increase in protein content, or at least a 4% increase in protein content, or at least a 6% increase in protein content, or at least an 8% increase in protein content, or at least 10% increase in protein content, or at least a 20% increase in protein content, or at least a 50% increase in protein content in its fruit, seeds, or harvested parts as compared to control crop plants not harboring the genetic modification.

In a further embodiment of the invention, the selected plant is a soybean, maize, rice, potato, tomato, or wheat plant.

In a further embodiment of the above invention the crop plant is a soybean plant and the main ORF encodes a polypeptide that has at least 70% identity to, or is identical to the sequence:

METNNQQQQQQGAQAQSGPYPVAGAGGSAGAGAGAPPPFQHLLQQQQQQ
LQMFWSYQRQEIEHVNDFKNHQLPLARIKKIMKADEDVRMISAEAPILF
AKACELFILELTIRSWLHAEENKRRTLQKNDIAAAITRTDIFDFLVDIV
PRDEIKDDAALVGATASGVPYYYPPIGQPAGMMIGRPAVDPATGVYVQP
PSQAWQSVWQSAAEDASYGTGGAGAQRSLDGQS*

In a further embodiment of the above invention the crop plant is a maize plant and the main ORF encodes a polypeptide that has at least 70% identity to, or is identical to, the maize NF-YC4 sequence:

MDNQPLPYSTGQPPAPGGAPVAGMPGAAGLPPVPHHHLLQQQAQLQAFW
AYQRQEAERASASDFKNHQLPLARIKKIMKADEDVRMISAEAPVLFAKA
CELFILELTIRSWLHAEENKRRTLQRNDVAAAIARTDVFDFLVDIVPRE
EAKEEPGSALGFAAPGTGVVGAGAPGGAPAAGMPYYYPPMGQPAPMMPA
WHVPAWDPAWQQGAADVDQSGSFSEEGQGFGAGHGGAASFPPAPPTSE*

In a further embodiment of the above invention the crop plant is a wheat plant and the main ORF encodes a polypeptide that has at least 70% identity to, or is identical to, the wheat NF-YC4 sequence:

MENHQLPYTTQPPATGAAGGAPVPGVPGPPPVPHHHLLQQQQAQLQAFW
AYQRQEAERASASDFKNHQLPLARIKKIMKADEDVRMISAEAPVLFAKA
CELFILELTIRSWLHAEENKRRTLQRNDVAAAIARTDVFDFLVDIVPRE
EAKEEPGSAALGFAAGGVGAAGGGPAAGLPYYYPPMGQPAAPMMPAWHV
PAWEPAWQQGGADVDQGAGSFGEEGQGYTGGHGGSAGFPPGPPSSD*

In a further embodiment of the above invention the crop plant is a rice plant and the main ORF encodes a polypeptide that has at least 70% identity to, or is identical to, the rice NF-YC4 sequence:

MDNQQLPYAGQPAAAGAGAPVPGVPGAGGPPAVPHHHLLQQQQAQLQAF
WAYQRQEAERASASDFKNHQLPLARIKKIMKADEDVRMISAEAPVLFAK
ACELFILELTIRSWLHAEENKRRTLQRNDVAAAIARTDVFDFLVDIVPR
EEAKEEPGSALGFAAGGPAGAVGAAGPAAGLPYYYPPMGQPAPMMPAWH
VPAWDPAWQQGAAPDVDQGAAGSFSEEGQQGFAGHGGAAASFPPAPPSS
E*

In a further embodiment of the above invention the crop plant is a tomato plant and the main ORF encodes a polypeptide that has at least 70% identity to, or is identical to, the tomato NF-YC4 sequence:

MDNQQLPYAGQPAAAGAGAPVPGVPGAGGPPAVPHHHLLQQQQAQLQAF
WAYQRQEAERASASDFKNHQLPLARIKKIMKADEDVRMISAEAPVLFAK
ACELFILELTIRSWLHAEENKRRTLQRNDVAAAIARTDVFDFLVDIVPR
EEAKEEPGSALGFAAGGPAGAVGAAGPAAGLPYYYPPMGQPAPMMPAWH
VPAWDPAWQQGAAPDVDQGAAGSFSEEGQQGFAGHGGAAASFPPAPPSS
E*

In a further embodiment of the above invention the crop plant is a potato plant and the main ORF encodes a polypeptide that has at least 70% identity to, or is identical to, the potato NF-YC4 sequence:

MDNNPHQSPTEAAAAAAAAAAAAQSATYPPQTPYHHLLQQQQQQLQMFW
TYQRQEIEQVNDFKNHQLPLARIKKIMKADEDVRMISAEAPVLFAKACE
LFILELTIRSWLHAEENKRRTLQKNDIAAAITRTDIFDFLVDIVPRDEI
KDEGVVLGPGIVGSTASGVPYYYPPMGQPAPGGVMLGRPAVPGVDPSMY
VHPPPSQAWQSVWQTGDDNSYASGGSSGQGNLDGQI*

In a further embodiment of the above invention the crop plant is a plant of the genus Gossypium and the main ORF encodes a polypeptide that has at least 70% identity to, or is identical to, the Gossypium NF-YC4 sequence:

MDSNQQTQSTPYPPQPPTSAITPPSSATATAPPFHHLLQQQQQQLQMFW
SYQRQEIEQVNDFKNHQLPLARIKKIMKADEDVRMISAEAPILFAKACE
LFILELTIRSWLHAEENKRRTLQKNDIAAAITRTDIFDFLVDIVPRDEI
KDETGLAPMVGATASGVPYFYPPMGQPAAGGPGGMMIGRPAVDPTGGIY
GQPPSQAWQSVWQTAGTDDGSYGSGVTGGQGNLDGQG*

In a further embodiment of the above invention the crop plant is a plant that is grown for animal forage or silage, for example, alfalfa, Sorghum, or a forage grass species.

In a further embodiment of the above invention, the plant is a species grown as protein source for human consumption, for example pea, pulses, bean, or chickpea.

In a yet further embodiment of the invention, a NF-YC4 group transcription factor is expressed in a transgenic plant, but the approach is improved by incorporating a form of the gene encoding the NF-YC4 transcription factor into the expression construct, which has a mutation or deletion within, or of, a uORF sequence in the 5′ UTR of the gene upstream of the main ORF that encodes the NF-YC4 TF. It is notable that prior attempts to overexpress members of this family of TFs may have been hampered by the inadvertent inclusion, by the practitioners, of uORF sequences upstream of the main ORF of the target gene being expressed. Thus, when the transgene transcript was produced, its translation would have been repressed by the presence of the uORF. By intentionally omitting or mutating such uORFs, in this example, such inadvertent repression of translation is avoided and an enhancement to the desired phenotype is obtained.

By way of further illustration to the above example, researchers have reported phenotypes of transgenic soybean and corn lines expressing NF-YC4 subunits, but the resulting plants exhibited around 40% or less seed protein in the case of soybean and a protein content of around 120 mg/g dry weight or less in the corn, based on a Lowry test (O'Conner et al. Book Chapter 6. “From Arabidopsis to Crops: The Arabidopsis QQS Orphan Gene Modulates Nitrogen Allocation across species.” In: “Engineering Nitrogen Utilization in Crop Plants.” Edited by Shrawat, Zayed and Lightfoot. Springer 2018). Furthermore, these authors did not note any striking increase in size or vigor of the transgenic plants compared to controls. Through overexpression of a variants of such transgenes that lack a uORF, or possess a mutated uORF, in the 5′ UTR, an improvement in the trait may be obtained. In a particular embodiment of this example, the improved trait in soybean is a seed protein content of greater than approximately 40% and/or the soybean plants exhibiting a greater size than controls. In a further embodiment relating to corn, the improved trait is a protein content of the seed that is greater than approximately 120 mg/g fresh weight and/or the corn plants exhibiting a greater size than controls.

### Example 17

Use of uORFs to Optimize Transgene Activity in Transgenic Organisms.

Methods for generating transgenic organisms from many species, spanning plants, animals, and microbes have been relatively for several decades. However, practitioners of these approaches face a common challenge; that of optimizing the dosage of the transgene product. Typically, a transformation method involves building a DNA construct containing the transgene of interest regulated by a heterologous promoter, or multiple copies of the transgene. The construct is then introduced into a cell of the target species, which optionally, may be selected and regenerated into a tissue or whole organism. The promoter included in the transgene construct will produce either a higher level of RNA from the transgene in a transformed cell than in a control cell or a tissue specific or a conditionally inducible pattern of expression of the transgene RNA. However, often, the level of translation of the resulting RNA cannot be precisely controlled.

Mutation of uORFs to Elevate the Expression of a Transgene or a Target Gene at its Native Locus

In some instances, a uORF represses translation from the native transcript of the gene. In such instances, a practitioner may upregulate the gene by mutating the uORF at the native locus or by overexpressing the gene using transgenic approach. In such instances, if an unrecognized uORF is present in the transgene transcript upstream of the main ORF, this can cause repression of translation and failure of the transgene to deliver the target trait. In such instances, application of the methods described herein can identify the presence of uORF(s) in the transgene 5′ region and these can be intentionally omitted, or mutated by TILLING or by gene editing if a native locus is being targeted) to weaken or remove the uORF function and enable translation of the transgene product.

A specific example of the use of this method is in optimizing activity of the REVOLUTA class of HD-ZIP class III transcription factors, of which (REV/IFL1) was the founding member. At least 5 closely related members of this clade of transcription factors are encoded by the Arabidopsis genome (Locus identifiers: AT1G30490, AT4G32880, AT2G34710, AT5G60690 and AT1G52150). The activity of these genes, and their encoded polypeptides, may be upregulated by TILLING or gene editing to obtain alleles that produce elevated levels of the proteins leading to a Trait of Interest.

The REVOUTA (REV) clade of transcription factors has critical roles in regulation of meristem behavior and development, including adaxial/abaxial patterning. When knocked out in a homozygous state, loss of function rev mutants in Arabidopsis show abnormalities in shoot morphology, including a lack of interfascicular fibers in the stem, reduced outgrowth of secondary shoot meristems, and elongated twisted leaves. If a practitioner attempts to overexpress a gene from this group and includes the native uORF upstream of the main ORF within the transgene construct downstream of the transgene promoter, the resulting transformed plants typically display a wild-type phenotype. However, if the practitioner intentionally omits the native uORF from the DNA clone, included in the transgene construct, or includes a weakened uORF variant with sequence changes versus the native uORF, the resulting transformed plants typically display one or more Improved Traits, which may include increased yield or increased biomass yield.

Additionally, one or more Improved Traits may be obtained by generating alleles through gene editing or TILLING that comprise mutations that disrupt the native uORF in one or more genes of the REV class of transcription factors at their native loci in the plant genome. As a specific example, a mutation in a uORF of a tomato gene encoding a REV homolog may be mutated through gene editing or TILLING to produce one or more Improved Traits, which may include altered leaf shape, a more compact shoot system, and/or increased yield.

Introduction of a uORF to Dampen Expression

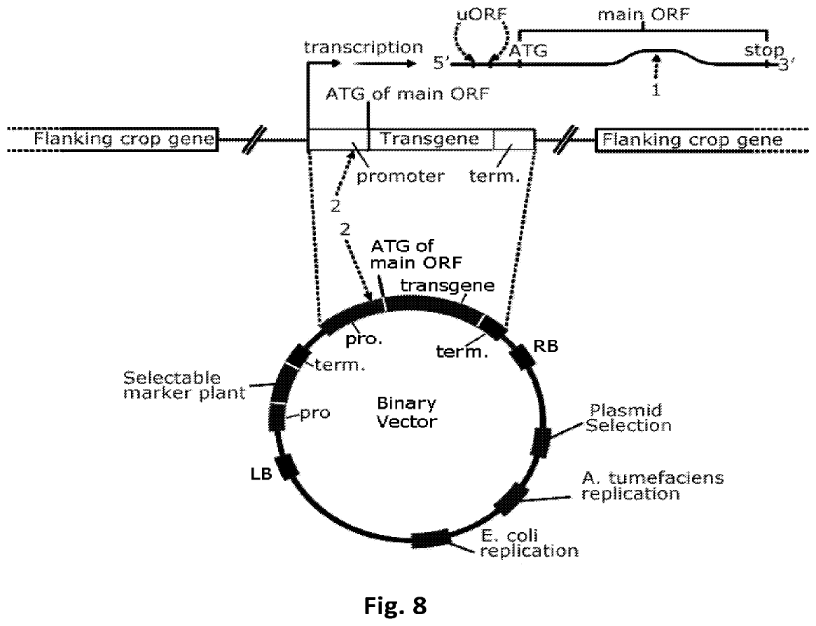

Conversely, in transgenes that lack a “strong” uORF, a higher than optimal level of translation may produce a higher than necessary dose of the polypeptide produced by the transgene, resulting in undesirable side effects (or “off-types”), in addition to the trait of interest. Such side effects may include dwarfing, slow growth, and developmental abnormalities such as defective tissues and/or misshapen organs. In these instances, a uORF may be introduced into the 5′ region of the transgene, upstream of the start codon of the main ORF to provide a mechanism to dampen translation of the encoded protein to a more optimal level (FIG. 8). Importantly, a uORF may be introduced initially, at the time of design of the transgene construct, or after the fact, once a transgenic line or event of an organism has been selected, which harbors the transgene integrated at some particular locus in its genome, and which shows a desired phenotype, but also has undesirable off-types.

The above approach may be applied to improve or optimize existing transgenic crop events, which have publicly described, and/or which have been deregulated by, or which have been submitted for deregulation by, the USDA APHIS, which oversees the release of transgenic crops in the US. Crop events to which this approach may be applied include ZmNF-YB2 drought tolerant corn [developed by Monsanto Company®, now Bayer CropScience®, see: Nelson et al. (2007). PNAS 104 no. 42, 16450-16455], BBX32 soybean [developed by Monsanto Company, now Bayer CropScience, see: Preuss S B, Meister R, Xu Q, Urwin C P, Tripodi F A, et al. (2012) Expression of the Arabidopsis thaliana BBX32 Gene in Soybean Increases Grain Yield. PLoS ONE 7(2): e30717. doi:10.1371/journal.pone.0030717], ATHB17 corn [developed by Monsanto Company, now Bayer CropScience, see Rice E A, Khandelwal A, Creelman R A, Griffith C, Ahrens J E, et al. (2014) Expression of a Truncated ATHB17 Protein in Maize Increases Ear Weight at Silking. PLoS ONE 9(4): e94238. doi:10.1371/journal.pone.0094238], ZMM28 corn [developed by Corteva Agriscience, see: Wu et al. (2019), PNAS vol. 116, no. 47, 23851], and/or crops transformed with the drought tolerance conferring genes HaHB4, ATHB13 or ATHB7 or their homologs, and/or crops transformed with the stress tolerance conferring genes CBF1-4 or their homologs (CBF1=AT4G2549, CBF2=AT4G25470, CBF3=AT4G25480, CBF4=AT5G51990; BBX32=AT3G21150; ATHB17=AT2G01430; ATHB13=AT1G69780; ATHB7=AT2G46680).

In the above and comparable instances, the practitioner selects a uORF sequence that is at a least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to a uORF identified herein (SED ID NO. 2n−1, where n=1-1999 and SEQ ID NO: 5156-5227) or creates an artificial uORF, that is an open reading frame (comprising a stretch of nucleotides that begins with a start codon and ends with a stop codon, of approximately 10-300 bp in overall length) that is located upstream of, the main ORF (typically 10-500 bp upstream of the main ORF ATG, but longer or shorter distances may also be effective), and introduces the selected uORF into the transgene construct (which is subsequently introduced in a plant cell), or into the genome by gene editing, in the case of an existing stable crop event that is being engineered. The practitioner then selects a plant from amongst the resultant transformants or gene edited lines which shows an improvement in an Improved Trait (as defined herein) as compared to a control plant, (which may be a wild-type plant, or a plant of the original event, in the case where an existing transformed line is being optimized). For example, BBX32 soybean lines may be selected from a population which exhibit improved yield without delayed maturation. Similarly, gene edited ATHB17 or ZMM28 corn lines with introduced uORFs may be selected which have an even greater improvement in yield as compared to the original transgenic event, respectively, or in the case of ZMM28, a reduction in the delay of heat units to silking as described by Wu et al., supra. In the case of ZmNF-YB2 transgenic corn events expressing this transcription factor show marked yield increases compared to controls in non-irrigated dry fields but when grown in well-watered fields, the events show a reduced yield (so called “yield drag”) compared to controls. In the example presented here, a uORF may be introduced into the ZmNF-YB2 transgene (or a transgene encoding a homologous protein, including those describe by Nelson 2007, supra) to obtain improved yield in dry fields while reducing or eliminating the yield drag observed in irrigated conditions.

### Example 18

Introduction of uORFs Through Gene Editing to Generate “Knock-Down” Alleles of Target Genes.

uORFs may also be used a tool to knock-out or knock down a target gene by introducing them through gene editing into the 5′ region of the main ORF of the target gene by gene editing.

A particular example concerns the HY5 related transcription factors and their bZIP family homologs which promote photomorphogenesis. Preuss et al. supra, and Khanna et al. reported that BBX32 represses light signaling through inhibition of other BBX family proteins, as well as repression of HY5, which in species like soybean, results in beneficial features such as increased root growth, increased pod number and/or a yield increase. Thus, introduction of uORFs into the 5′ regions of these genes encoding HY5 homologs by gene editing, particularly in soybean, may produce an Improved Trait such as the aforementioned phenotypes.

In the above and comparable instances, the practitioner selects a uORF sequence that is at a least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to a uORF identified herein (SED ID NO. 2n−1, where n=1-1999 and SEQ ID NO: 5156-5227), or creates an artificial uORF by inserting it into the genome by gene editing, that is an open reading frame (comprising a stretch of nucleotides that begins with a start codon and ends with a stop codon, of approximately 10-300 bp in overall length) that is located upstream of the main ORF. The practitioner then selects a plant from amongst gene edited lines which shows an improvement in an Improved Trait (as defined herein) as compared to a control plant.

### Example 19

Use of a uPEP as a Biostimulant

In cases where a uORF is identified in the 5′ transcript of a gene containing a main ORF that promote a beneficial phenotype when its activity is reduced or knocked out, the uPEP encoded by the uORF may be exogenously applied as a biostimulant to obtain a desired trait. The desired trait may be improved drought tolerance, improved yield or an Improved Trait as detailed herein.

### Example 19A

A method of obtaining an Improved Trait in a plant comprising: first selecting a uORF sequence that is at a least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to a uORF identified herein (SED ID NO. 2n−1, where n=1-1999 and SEQ ID NO: 5156-5227), and introducing the uORF into an expression vector that enables the production of the encoded uPEP in a cell or tissue, via a process such as through fermentation. The expression vector is then introduced into a cell or tissue and the uPEP produced is harvested, processed, formulated and applied to a plant as a biostimulant.

### Example 19B

The method of EXAMPLE 19 wherein the uORF is selected from a gene, the main ORF of which encodes a homolog of the bZIP protein HY5 (AT5G11260). In a particular embodiment of this example, the uORF is derived from the HY5 locus or from a soybean homolog and a formulation of the resulting uPEP is sprayed onto soybean plants, leading to an improvement in yield.

### Example 19C

A method of inducing flowering in a crop comprising identifying a uORF in the 5′ region of a gene the main ORF of which represses flowering, introducing the uORF into an expression vector that enables the production of the encoded uPEP in a cell or tissue, such as through fermentation, introducing the expression vector into a cell or tissue, harvesting the uPEP produced from the cell or tissue and applying a formulation containing the uPEP to a vegetatively growing plant.

### Example 19D

The method of 19C wherein the main ORF encodes a homolog of CCA1 (AT2G46830), LHY (AT1G01060), FLC (AT5G10140) or TERMINAL FLOWER 1 (AT5G03840).

### Example 19E

A method of repressing or delaying flowering, or producing sterility in a crop, the method comprising identifying a uORF in the 5′ region of a gene the main ORF of which promotes flowering or floral organ development, introducing the uORF into an expression vector that enables the production of the encoded uPEP in a cell or tissue, such as through fermentation, introducing the expression vector into a cell or tissue, harvesting the uPEP produced from the cell or tissue and applying a formulation containing the uPEP to a vegetatively growing plant.

In a further embodiment of this example, the practitioner applies one or more treatments of the uPEP to the plant thereby delaying the floral transition and enabling the plant to accumulate a greater amount of photosynthetic biomass, and hence a greater yield, once treatments of the uPEP have ceased.

### Example 19F

The method of 19C wherein the main ORF encodes a homolog of CONSTANS (AT5G15840), SOC1 (AT2G45660), FLOWERING LOCUS T (AT1G65480), LEAFY (AT5G61850), FCA (AT4G16280), GIGANTEA (AT1G22770), PISTILLATA (AT5G20240), APETALA3 (AT3G54340), AGAMOUS (AT4G18960), CAULIFLOWER (AT1G26310) or APETALA 1 (AT1G69120).

The present invention is not limited by the specific embodiments described herein. The invention now being fully described, it will be apparent to one of ordinary skill in the art that many changes and modifications can be made thereto without departing from the spirit or scope of the Claims. Modifications that become apparent from the foregoing description and accompanying figures fall within the scope of the following Claims.

1-30. (canceled)

31. A method of identifying the presence of a putative upstream open reading frame (uORF) in a polynucleotide sequence in a genome of an organism through application of an algorithm to ribosome profiling data, the method including:
a) identifying an existing or putative main Open Reading Frame (main ORF) in the genome of the organism and obtaining ribosome profiling data derived from mRNA transcribed from the genome of the organism, wherein said identifying may be performed de novo or from extant knowledge;
b) evaluating ribosome occupancy of at least one region of the genome that is upstream of the main open reading frame;
c) identifying a location of the genome upstream of the main ORF where there is ribosome enrichment and downstream of said enrichment there is an abrupt drop off in ribosome occupancy;
d) identifying a stop codon of a putative uORF in the genome at, or near to, the abrupt drop off in ribosome occupancy at the location;
e) identifying a second stop codon upstream and in frame with the stop codon of the putative uORF identified in d); and
f) wherein the algorithm identifies presence of a putative uORF within the genome in the interval between the first stop codon of the putative uORF and the second upstream stop codon within the same open reading frame.

32. The method of claim 31, wherein a targeted genetic modification is introduced into the putative uORF
to create a modified uORF and translation of the main ORF operably linked to the modified uORF is increased.

33. The method of claim 32, where the modified uORF is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to the putative uORF.

34. The method of claim 32, wherein the modified uORF is reduced in function or knocked out by introducing at least one gene edit in the uORF.

35. The method of claim 32, wherein the modified uORF is reduced in function or knocked out in a plant, and the reduction or loss of function of the uORF nucleotide sequence results in increased translation of a main ORF that is operably linked to the uORF.

36. The method of claim 35, wherein the increased translation of the main ORF confers cell death, inhibition of cell division, or an Improved Trait selected from the group consisting of:
a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic
rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level;
as compared to a reference or control plant of the same species.

37. The method of claim 35, wherein the uORF regulates the translation of the main ORF and the increased translation of the main ORF results in a toxic effect or cell death or earlier flowering time, delayed flowering time, or bolting as compared to a reference or control plant of the same species.

38. A plant or plant cell comprising:
an introduced targeted genetic modification at a native genomic locus, wherein the introduced targeted genetic modification comprises a mutation in a uORF, wherein the native genomic locus comprises a main ORF operably linked to the uORF, wherein the main ORF encodes a polypeptide with regulatory activity, wherein the polypeptide comprises an amino acid sequence with a percentage identity to a polypeptide selected from the group consisting of SEQ ID NO: 3999-5155.
wherein the percentage identity is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100%; and
the targeted genetic modification to the uORF increases expression level and/or activity of the encoded polypeptide with regulatory activity.

39. The plant or plant cell of claim 38, wherein the introduced targeted genetic modification of the uORF results in increased translation of the main ORF operably linked to the uORF.

40. The plant or plant cell of claim 38, where the uORF with the targeted genetic modification is at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identical to a native uORF within the native genomic locus.

41. The plant or plant cell of claim 38, wherein the uORF comprising the introduced targeted genetic modification comprises at least one gene edit.

42. The plant or plant cell of claim 38, wherein the uORF with the introduced targeted genetic modification is reduced in function or knocked out in the plant and the modification of the uORF results in increased translation of a polypeptide-encoding main ORF that is operably linked to the uORF.

43. The plant or plant cell of claim 38, wherein the main ORF encodes a polypeptide the expression of which confers cell death, inhibition of cell division, or an Improved Trait selected from the group consisting of:
a yield increase, improved flavor, improved texture, altered circadian rhythm, accelerated flowering, accelerated senescence, delayed senescence, increased branching, reduced branching, increased apical dominance, reduced apical dominance, shade tolerance, increased root mass, increased root hair number, increased vegetative mass, increased fruit mass, improved fruit quality, increased germination rate, increased trichome length, reduced trichome length, reduced thorns, reduced spines, thornless, altered leaf shape, increased leaf number, reduced leaf number, altered leaf angle, altered leaf position, altered branch angle, increased peelability, reduced cellular adhesion, increased cellular adhesion, reduced peel thickness, reduced fruit skin thickness, increased fruit skin thickness, increased seed coat thickness, reduced seed coat thickness, seedless, reduced seed size, increased seed size, apomixis, increased embryogenesis, increased susceptibility to transgenic transformation, increased callus formation, increased embryo formation, increased root formation, increased cell division, reduced cell division, sterility, male sterility, inviable pollen, lack of stamens, lack of carpels, increased carpel number, increased petal number, reduced petal number increased trichome number, reduced trichome number, increased stem width, reduced stem width, increased internode length, reduced internode length, increased floral organ size, altered floral organ shape, reduced floral organ size, reduced fruit abscission, reduced pod shattering, altered organ abscission, variegation, increased hypocotyl length, reduced hypocotyl length, delayed flowering, elimination of flowering, sterility, improved osmotic stress tolerance, improved photosynthesis, improved nitrogen use efficiency, improved phosphorus use efficiency, improved potassium use efficiency, increased nutrient use efficiency, increased nutrient uptake, increased uptake of a metal ion, increased sequestration of a heavy metal, improved oxidative stress tolerance, increased pigment level, improved salt tolerance, improved cold tolerance, improved tolerance of freezing damage, improved freezing tolerance, improved dehydration stress, drought tolerance, improved recovery following drought, decreased wilting, increased plastid number, increased chlorophyll content, increased thylakoid density, increased photosynthetic capacity, increased respiration, reduced respiration, increased photorespiration, reduced photorespiration, increased transpiration, reduced transpiration, increased stomatal conductivity, reduced stomatal conductivity, increased carbon fixation, increased carbon sequestration, increased photosynthetic rate, increased carotenoid level, reduced carotenoid level, increased electron transport, improved non-photochemical quenching, increased ion transport, reduced ion transport, altered carbon to nitrogen balance, increased sensitivity to a hormone, reduced sensitivity to a hormone, reduced sensitivity to ethylene, increased auxin level, reduced auxin level, increased auxin transport, increased auxin sensitivity, reduced auxin sensitivity, increased gibberellin level, reduced gibberellin increased gibberellin sensitivity, reduced gibberellin sensitivity, increased abscisic acid level, reduced abscisic acid level, increased abscisic acid sensitivity, reduced abscisic acid sensitivity, increased cytokinin level, reduced cytokinin level, increased cytokinin level, reduced cytokinin level, increased cytokinin sensitivity, reduced cytokinin sensitivity, increased jasmonate level, reduced jasmonate level, increased jasmonate sensitivity, reduced jasmonate sensitivity, increased salicylic acid level, reduced salicylic acid level, reduced salicylic acid sensitivity, increased salicylic acid sensitivity, increased strigolactone level, reduced strigolactone level, increased sensitivity to strigolactone, reduced sensitivity to strigolactone, reduced sensitivity to ethylene, increased sensitivity to ethylene, accelerated ripening, delayed ripening, reduced fruit spoilage, increased shelf life, improved heat stress, improved tolerance to low nitrogen conditions, increased seedling vigor, increased disease resistance, increased resistance to a fungal pathogen, increased resistance to bacterial pathogen, increased resistance to a viral pathogen, increased resistance to Botrytis; increased resistance to Erysiphe; increased resistance to Fusarium; increased resistance to Sclerotinia; increased rust resistance, increased resistance to Phytophthora, increased resistance to black sigatoka, increased resistance to Xanthomonas, increased resistance to a necrotrophic fungus, increased resistance to a biotrophic fungus, increase nematode resistance, increased insect resistance, herbivore resistance, increased mollusk resistance, increased protein levels, increased oil levels, reduced lignin level, increased THC level, increased CBD level, increased anthocyanin level, reduced anthocyanin level, increased nutrient level in tissue, increased vitamin level in tissue, increased carbohydrate level, reduced level of a carbohydrate, increased starch level, increased sugar level, increased BRIX, increased protein level, reduced protein level, increased level of a metabolite, increased level of photosynthetic pigments, increased lipid level, reduced lipid level, altered fatty acid saturation, increased level of saturated fat, reduced level of saturated fat, increased tocopherol level, reduced tocopherol level, increased prenyl lipid levels, increased nutritional content of tissues, increased processability, increased calorific value, reduced levels of chlorine, increased alkaloid level, reduced alkaloid level, increased wax level, reduced wax level, increased wax ester level, increased tannin level, increased taxol level, increased xanthophyll levels, increased bioplastic levels, increased level of a biopolymer, reduced levels or a biopolymer, altered starch composition, increased latex level, and increased rubber level;
as compared to a reference or control plant of the same species

44. The method of claim 42, wherein the introduced targeted genetic modification results in increased translation of the main ORF which results in a toxic effect or cell death or earlier flowering time, delayed flowering time, or bolting as compared to a reference or control plant of the same species.

45. A plant, the genome of which contains a non-naturally occurring allele of a gene comprising a mutation in a uORF upstream of a main ORF which encodes a protein that is a Homolog of, or which has at least 30% or at least 35%, or at least 40%, or at least 45%, or at least 50%, or at least 55%, or at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%, or about 100% identity to, the protein encoded by CCA1 (SEQ ID NO: 4483; Arabidopsis locus AT2G46830) and wherein the plant is selected for an Improved Trait.

46. The plant of claim 45, wherein the Improved Trait is delayed flowering, increased photosynthesis, increased vegetative biomass, a more compact shoot structure, or increased yield.
